(19) United States
(12) Patent Application Publication
(10) Pub. No.: US 2025/0072273 A1
(43) Pub. Date: Feb. 27, 2025

(54) ORGANIC ELECTROLUMINESCENCE ELEMENT, ELECTRONIC DEVICE, COMPOSITION, AND MIXTURE POWDER

(71) Applicant: IDEMITSU KOSAN CO.,LTD., Tokyo (JP)

(72) Inventors: Satomi TASAKI, Tokyo (JP); Hiroaki TOYOSHIMA, Tokyo (JP); Tetsuya MASUDA, Tokyo (JP); Masato NAKAMURA, Tokyo (JP); Hiroaki ITOI, Tokyo (JP)

(73) Assignee: IDEMITSU KOSAN CO.,LTD., Tokyo (JP)

(21) Appl. No.: 18/722,535

(22) PCT Filed: Dec. 19, 2022
(86) PCT No.: PCT/JP2022/046706

§ 371 Date: Jun. 20, 2024

(30) Foreign Application Priority Data
Dec. 21, 2021 (JP) 2021-207567

(51) Int. Cl. H10K 85/20; C09K 11/02; C09K 11/06; H10K 85/40; H10K 85/60; H10K 101/00; H10K 101/10; H10K 101/30

(52) U.S. Cl. CPC H10K 85/20; C09K 11/02; C09K 11/06; H10K 85/40; H10K 85/622; H10K 85/623; H10K 85/633; H10K 85/653; H10K 85/656; H10K 85/6572; H10K 85/6574; H10K 85/6576; H10K 85/658; H10K 2101/10; H10K 2101/30; H10K 2101/90

(57) ABSTRACT

An organic EL device includes an emitting region and a first anode side organic layer. The first anode side organic layer contains a first material. The emitting region includes a first emitting layer containing first and second host materials and a second emitting layer containing a third host material. Energy levels of highest occupied molecular orbitals of the first material, the first host material, and the second host material HOMO(HT1), HOMO(H1), and HOMO(H2) satisfy a relationship of a numerical formula (Numerical Formula A1) below, and triplet energies of the first host material, the second host material, and the third host material T₁(H1), T₁(H2), and T₁(H3) satisfy relationships of numerical formulae (Numerical Formula A2 and Numerical Formula A3) below,

$$HOMO ( HT 1 ) > HOMO ( H 2 ) > HOMO ( H 1 ) \qquad ( Numerical Formula A1 )$$

$${T}_{1} ( H 1 ) > {T}_{1} ( H 3 ) \qquad ( Numerical Formula A2 )$$

$${T}_{1} ( H 2 ) > {T}_{1} ( H 3 ) . \qquad ( Numerical Formula A3 )$$

### TECHNICAL FIELD

The present invention relates to an organic electroluminescence device, an electronic device, a composition, and a mixture powder.

### BACKGROUND ART

An organic electroluminescence device (hereinafter, occasionally referred to as “organic EL device”.) has found its application in a full-color display for mobile phones, televisions, and the like. When voltage is applied to an organic EL device, holes are injected from an anode and electrons are injected from a cathode into an emitting layer. The injected holes and electrons are recombined in the emitting layer to form excitons. Specifically, according to the electron spin statistics theory, singlet excitons and triplet excitons are generated at a ratio of 25%:75%.

In order to enhance performance of the organic EL device, for instance, layering a plurality of emitting layers has been studied in Patent Literature 1. Further, in order to enhance performance of the organic EL device, Patent Literature 2 describes a phenomenon in which a singlet exciton is generated by collision and fusion of two triplet excitons (hereinafter, occasionally referred to as a Triplet-Triplet Fusion (TTF) phenomenon).

The performance of the organic EL device is evaluable in terms of, for instance, luminance, emission wavelength, chromaticity, luminous efficiency, drive voltage, and lifetime.

### CITATION LIST

### Patent Literature(s)

Patent Literature 1 US Patent Application Publication No. 2019/280209 Patent Literature 2 International Publication No. WO 2010/134350

### SUMMARY OF THE INVENTION

### Problem(s) to be Solved by the Invention

An object of the invention is to provide an organic electroluminescence device capable of maintaining luminous efficiency and having a long lifetime, to provide a composition and a mixture powder usable in the organic electroluminescence device, and an electronic device including the organic electroluminescence device.

### Means for Solving the Problem(s)

According to an aspect of the invention, there is provided an organic electroluminescence device, including:
an anode;
a cathode;
an emitting region disposed between the anode and the cathode; and
a first anode side organic layer disposed between the emitting region and the anode, in which
the first anode side organic layer contains a first material,
the emitting region includes a first emitting layer and a second emitting layer,
the first emitting layer is disposed between the first anode side organic layer and the second emitting layer,
the first emitting layer is in direct contact with the first anode side organic layer,
the first emitting layer contains a first host material and a second host material,
the second emitting layer contains a third host material,
an energy level of a highest occupied molecular orbital of the first material HOMO(HT1), an energy level of a highest occupied molecular orbital of the first host material HOMO(H1), and an energy level of a highest occupied molecular orbital of the second host material HOMO(H2) satisfy a relationship of a numerical formula (Numerical Formula A1) below,
a triplet energy of the first host material T₁(H1) and a triplet energy of the third host material T₁(H3) satisfy a relationship of a numerical formula (Numerical Formula A2) below, and
a triplet energy of the second host material T₁(H2) and the triplet energy of the third host material T₁(H3) satisfy a relationship of a numerical formula (Numerical Formula A3) below.

$$HOMO ( HT 1 ) > HOMO ( H 2 ) > HOMO ( H 1 ) \qquad ( Numerical Formula A1 )$$

$${T}_{1} ( H 1 ) > {T}_{1} ( H 3 ) \qquad ( Numerical Formula A2 )$$

$${T}_{1} ( H 2 ) > {T}_{1} ( H 3 ) \qquad ( Numerical Formula A3 )$$

According to another aspect of the invention, there is provided an organic electroluminescence device, including:
an anode;
a cathode; and
an emitting region disposed between the anode and the cathode, in which
the emitting region includes a first emitting layer,
the first emitting layer contains a first host material and a second host material,
the first host material is different from the second host material,
a triplet energy of the first host material T₁(H1) satisfies a relationship of a numerical formula (Numerical Formula A4) below, and
a triplet energy of the second host material T₁(H2) satisfies a relationship of a numerical formula (Numerical Formula A5) below.

$$1.8 eV < {T}_{1} ( H 1 ) < 2.5 eV \qquad ( Numerical Formula A4 )$$

$$1.8 eV < {T}_{1} ( H 2 ) < 2.5 eV \qquad ( Numerical Formula A5 )$$

According to still another aspect of the invention, there is provided a composition containing a first compound and a second compound, in which
the first compound and the second compound are mutually different compounds, and
the first compound and the second compound each independently include, in a molecule, at least one of a structure of Condition (i) or a structure of Condition (ii) below,
Condition (i): a biphenyl structure including a first benzene ring and a second benzene ring linked to each other with a single bond, the first benzene ring and the second benzene ring in the biphenyl structure being further linked to each other by cross-linking at at least one site other than the single bond; and
Condition (ii): a first linking structure including a benzene ring and a naphthalene ring linked to each other with a single bond, the benzene ring and the naphthalene ring in the first linking structure being each independently further fused or not fused with a monocyclic ring or fused ring, the benzene ring and the naphthalene ring in the first linking structure being further linked to each other by cross-linking at at least one site other than the single bond.

According to a further aspect of the invention, there is provided a mixture powder containing a first compound and a second compound, in which the first compound and the second compound are mutually different compounds, and the first compound and the second compound each independently include, in a molecule, at least one of the structure of Condition (i) or the structure of Condition (ii).

According to a still further aspect of the invention, there is provided a composition containing a first compound and a second compound, in which
the first compound and the second compound are mutually different compounds,
an energy level of a highest occupied molecular orbital of the first compound HOMO(C1) and an energy level of a highest occupied molecular orbital of the second compound HOMO(C2) satisfy a relationship of a numerical formula (Numerical Formula A7) below,
a triplet energy of the first compound T₁(C1) satisfies a relationship of a numerical formula (Numerical Formula A8) below, and
a triplet energy of the second compound T₁(C2) satisfies a relationship of a numerical formula (Numerical Formula A9) below.

$$HOMO ( C 2 ) > HOMO ( C 1 ) \qquad ( Numerical Formula A7 )$$

$$1.8 eV < {T}_{1} ( C 1 ) < 2.5 eV \qquad ( Numerical Formula A8 )$$

$$1.8 eV < {T}_{1} ( C 2 ) < 2.5 eV \qquad ( Numerical Formula A9 )$$

According to a still further aspect of the invention, there is provided a mixture powder containing a first compound and a second compound, in which the first compound and the second compound are mutually different compounds, an energy level of a highest occupied molecular orbital of the first compound HOMO(C1) and an energy level of a highest occupied molecular orbital of the second compound HOMO(C2) satisfy the relationship of the numerical formula (Numerical Formula A7), a triplet energy of the first compound T₁(C1) satisfies the relationship of the numerical formula (Numerical Formula A8), and a triplet energy of the second compound T₁(C2) satisfies the relationship of the numerical formula (Numerical Formula A9).

According to a still further aspect of the invention, there is provided an organic electroluminescence device, including:
an anode;
a cathode; and
an emitting region disposed between the anode and the cathode, in which
the emitting region includes a first emitting layer, and
the first emitting layer contains the composition according to the above aspect of the invention.

According to a further aspect of the invention, there is provided an electronic device including the organic electroluminescence device according to the above aspect of the invention.

According to the aspects of the invention, there are provided an organic electroluminescence device capable of maintaining luminous efficiency and having a long lifetime, to provide a composition and a mixture powder usable in the organic electroluminescence device, and an electronic device including the organic electroluminescence device.

### BRIEF EXPLANATION OF DRAWINGS

FIG. 1 schematically depicts an exemplary arrangement of an organic electroluminescence device according to a first exemplary embodiment.

FIG. 2 schematically depicts an exemplary arrangement of an organic electroluminescence device according to each of second, third, and fifth exemplary embodiments.

### DESCRIPTION OF EMBODIMENT(S)

### Definitions

Herein, a hydrogen atom includes isotope having different numbers of neutrons, specifically, protium, deuterium and tritium.

In chemical formulae herein, it is assumed that a hydrogen atom (i.e. protium, deuterium and tritium) is bonded to each of bondable positions that are not annexed with signs “R” or the like or “D” representing a deuterium.

Herein, the ring carbon atoms refer to the number of carbon atoms among atoms forming a ring of a compound (e.g., a monocyclic compound, fused-ring compound, cross-linking compound, carbon ring compound, and heterocyclic compound) in which the atoms are bonded to each other to form the ring. When the ring is substituted by a substituent(s), carbon atom(s) contained in the substituent(s) is not counted in the ring carbon atoms. Unless otherwise specified, the same applies to the “ring carbon atoms” described later. For instance, a benzene ring has 6 ring carbon atoms, a naphthalene ring has 10 ring carbon atoms, a pyridine ring has 5 ring carbon atoms, and a furan ring has 4 ring carbon atoms. Further, for instance, 9,9-diphenylfluorenyl group has 13 ring carbon atoms and 9,9′-spirobifluorenyl group has 25 ring carbon atoms.

When a benzene ring is substituted by a substituent in a form of, for instance, an alkyl group, the number of carbon atoms of the alkyl group is not counted in the number of the ring carbon atoms of the benzene ring. Accordingly, the benzene ring substituted by an alkyl group has 6 ring carbon atoms. When a naphthalene ring is substituted by a substituent in a form of, for instance, an alkyl group, the number of carbon atoms of the alkyl group is not counted in the number of the ring carbon atoms of the naphthalene ring. Accordingly, the naphthalene ring substituted by an alkyl group has 10 ring carbon atoms.

Herein, the ring atoms refer to the number of atoms forming a ring of a compound (e.g., a monocyclic compound, fused-ring compound, cross-linking compound, carbon ring compound, and heterocyclic compound) in which the atoms are bonded to each other to form the ring (e.g., monocyclic ring, fused ring, and ring assembly). Atom(s) not forming the ring (e.g., hydrogen atom(s) for saturating the valence of the atom which forms the ring) and atom(s) in a substituent by which the ring is substituted are not counted as the ring atoms. Unless otherwise specified, the same applies to the “ring atoms” described later. For instance, a pyridine ring has 6 ring atoms, a quinazoline ring has 10 ring atoms, and a furan ring has 5 ring atoms. For instance, the number of hydrogen atom(s) bonded to a pyridine ring or the number of atoms forming a substituent is not counted as the pyridine ring atoms. Accordingly, a pyridine ring bonded to a hydrogen atom(s) or a substituent(s) has 6 ring atoms. For instance, the hydrogen atom(s) bonded to carbon atom(s) of a quinazoline ring or the atoms forming a substituent are not counted as the quinazoline ring atoms. Accordingly, a quinazoline ring bonded to hydrogen atom(s) or a substituent(s) has 10 ring atoms.

Herein, “XX to YY carbon atoms” in the description of “substituted or unsubstituted ZZ group having XX to YY carbon atoms” represent carbon atoms of an unsubstituted ZZ group and do not include carbon atoms of a substituent(s) of the substituted ZZ group. Herein, “YY” is larger than “XX,” “XX” representing an integer of 1 or more and “YY” representing an integer of 2 or more.

Herein, “XX to YY atoms” in the description of “substituted or unsubstituted ZZ group having XX to YY atoms” represent atoms of an unsubstituted ZZ group and does not include atoms of a substituent(s) of the substituted ZZ group. Herein, “YY” is larger than “XX,” “XX” representing an integer of 1 or more and “YY” representing an integer of 2 or more.

Herein, an unsubstituted ZZ group refers to an “unsubstituted ZZ group” in a “substituted or unsubstituted ZZ group,” and a substituted ZZ group refers to a “substituted ZZ group” in a “substituted or unsubstituted ZZ group.”

Herein, the term “unsubstituted” used in a “substituted or unsubstituted ZZ group” means that a hydrogen atom(s) in the ZZ group is not substituted with a substituent(s). The hydrogen atom(s) in the “unsubstituted ZZ group” is protium, deuterium, or tritium.

Herein, the term “substituted” used in a “substituted or unsubstituted ZZ group” means that at least one hydrogen atom in the ZZ group is substituted with a substituent. Similarly, the term “substituted” used in a “BB group substituted by AA group” means that at least one hydrogen atom in the BB group is substituted with the AA group.

### Substituent Mentioned Herein

Substituent mentioned herein will be described below.

An “unsubstituted aryl group” mentioned herein has, unless otherwise specified herein, 6 to 50, preferably 6 to 30, more preferably 6 to 18 ring carbon atoms.

An “unsubstituted heterocyclic group” mentioned herein has, unless otherwise specified herein, 5 to 50, preferably 5 to 30, more preferably 5 to 18 ring atoms.

An “unsubstituted alkyl group” mentioned herein has, unless otherwise specified herein, 1 to 50, preferably 1 to 20, more preferably 1 to 6 carbon atoms.

An “unsubstituted alkenyl group” mentioned herein has, unless otherwise specified herein, 2 to 50, preferably 2 to 20, more preferably 2 to 6 carbon atoms.

An “unsubstituted alkynyl group” mentioned herein has, unless otherwise specified herein, 2 to 50, preferably 2 to 20, more preferably 2 to 6 carbon atoms.

An “unsubstituted cycloalkyl group” mentioned herein has, unless otherwise specified herein, 3 to 50, preferably 3 to 20, more preferably 3 to 6 ring carbon atoms.

An “unsubstituted arylene group” mentioned herein has, unless otherwise specified herein, 6 to 50, preferably 6 to 30, more preferably 6 to 18 ring carbon atoms.

An “unsubstituted divalent heterocyclic group” mentioned herein has, unless otherwise specified herein, 5 to 50, preferably 5 to 30, more preferably 5 to 18 ring atoms.

An “unsubstituted alkylene group” mentioned herein has, unless otherwise specified herein, 1 to 50, preferably 1 to 20, more preferably 1 to 6 carbon atoms.

### Substituted or Unsubstituted Aryl Group

Specific examples (specific example group G1) of the “substituted or unsubstituted aryl group” mentioned herein include unsubstituted aryl groups (specific example group G1A) below and substituted aryl groups (specific example group G1B). (Herein, an unsubstituted aryl group refers to an “unsubstituted aryl group” in a “substituted or unsubstituted aryl group”, and a substituted aryl group refers to a “substituted aryl group” in a “substituted or unsubstituted aryl group.”) A simply termed “aryl group” herein includes both of an “unsubstituted aryl group” and a “substituted aryl group”.

The “substituted aryl group” refers to a group derived by substituting at least one hydrogen atom in an “unsubstituted aryl group” with a substituent. Examples of the “substituted aryl group” include a group derived by substituting at least one hydrogen atom in the “unsubstituted aryl group” in the specific example group G1A below with a substituent, and examples of the substituted aryl group in the specific example group G1B below. It should be noted that the examples of the “unsubstituted aryl group” and the “substituted aryl group” mentioned herein are merely exemplary, and the “substituted aryl group” mentioned herein includes a group derived by further substituting a hydrogen atom bonded to a carbon atom of a skeleton of a “substituted aryl group” in the specific example group G1B below, and a group derived by further substituting a hydrogen atom of a substituent of the “substituted aryl group” in the specific example group G1B below.

Unsubstituted Aryl Group (Specific Example Group G1A): a phenyl group, p-biphenyl group, m-biphenyl group, o-biphenyl group, p-terphenyl-4-yl group, p-terphenyl-3-yl group, p-terphenyl-2-yl group, m-terphenyl-4-yl group, m-terphenyl-3-yl group, m-terphenyl-2-yl group, o-terphenyl-4-yl group, o-terphenyl-3-yl group, o-terphenyl-2-yl group, 1-naphthyl group, 2-naphthyl group, anthryl group, benzanthryl group, phenanthryl group, benzophenanthryl group, phenalenyl group, pyrenyl group, chrysenyl group, benzochrysenyl group, triphenylenyl group, benzotriphenylenyl group, tetracenyl group, pentacenyl group, fluorenyl group, 9,9′-spirobifluorenyl group, benzofluorenyl group, dibenzofluorenyl group, fluoranthenyl group, benzofluoranthenyl group, perylenyl group, and monovalent aryl group derived by removing one hydrogen atom from cyclic structures represented by formulae (TEMP-1) to (TEMP-15) below.

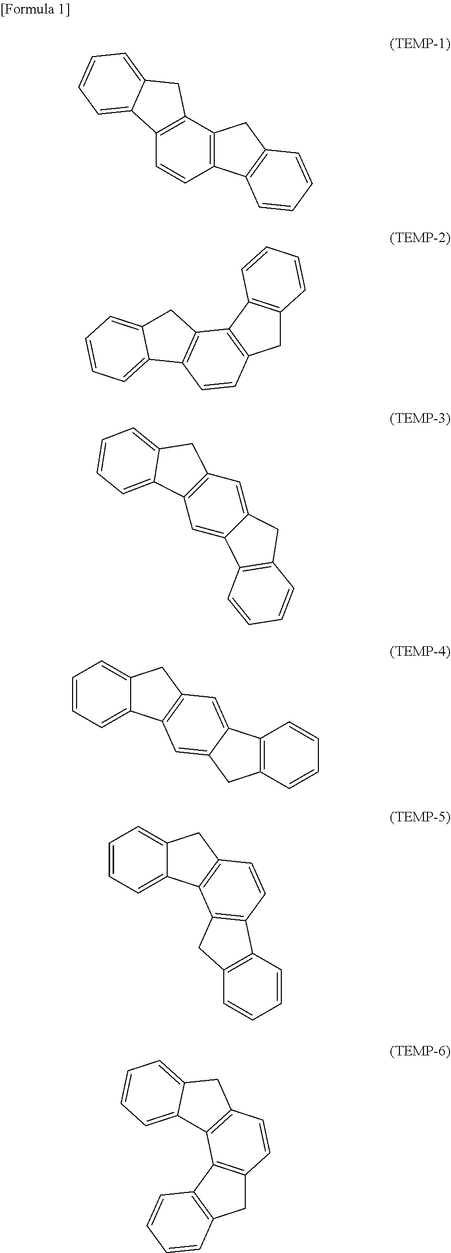

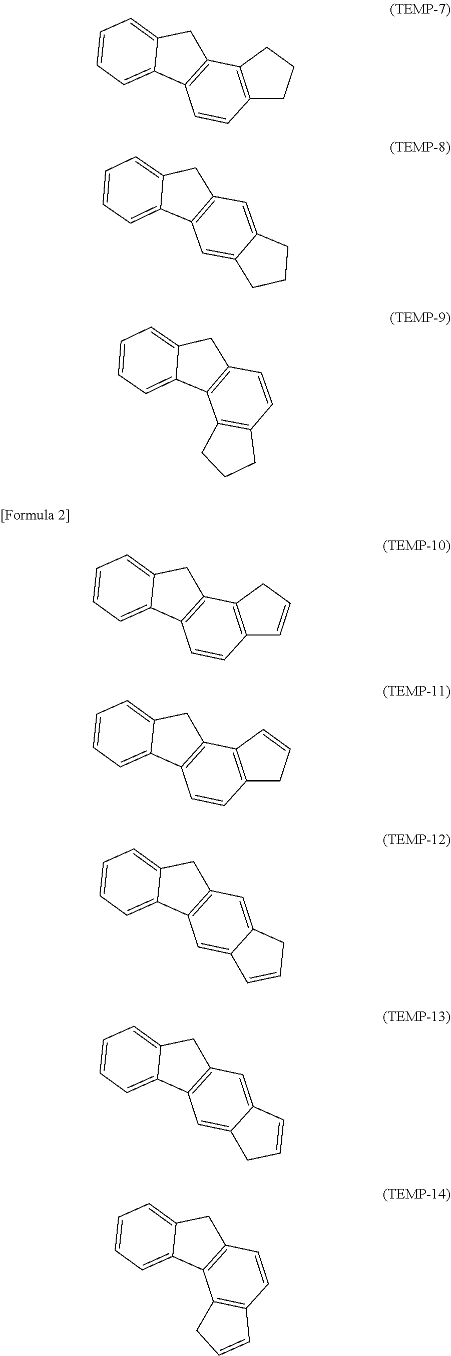

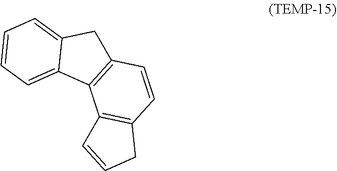 [SMILES: C1=Cc2c(ccc3c2-c2ccccc2C3)C1]

Substituted Aryl Group (Specific Example Group G1B): an o-tolyl group, m-tolyl group, p-tolyl group, para-xylyl group, meta-xylyl group, ortho-xylyl group, para-isopropylphenyl group, meta-isopropylphenyl group, ortho-isopropylphenyl group, para-t-butylphenyl group, meta-t-butylphenyl group, ortho-t-butylphenyl group, 3,4,5-trimethylphenyl group, 9,9-dimethylfluorenyl group, 9,9-diphenylfluorenyl group, 9,9-bis(4-methylphenyl) fluorenyl group, 9,9-bis(4-isopropylphenyl) fluorenyl group, 9,9-bis(4-t-butylphenyl) fluorenyl group, cyanophenyl group, triphenylsilylphenyl group, trimethylsilylphenyl group, phenylnaphthyl group, naphthylphenyl group, and group derived by substituting at least one hydrogen atom of a monovalent group derived from one of the cyclic structures represented by the formulae (TEMP-1) to (TEMP-15) with a substituent.

### Substituted or Unsubstituted Heterocyclic Group

The “heterocyclic group” mentioned herein refers to a cyclic group having at least one hetero atom in the ring atoms. Specific examples of the hetero atom include a nitrogen atom, oxygen atom, sulfur atom, silicon atom, phosphorus atom, and boron atom.

The “heterocyclic group” mentioned herein is a monocyclic group or a fused-ring group.

The “heterocyclic group” mentioned herein is an aromatic heterocyclic group or a non-aromatic heterocyclic group.

Specific examples (specific example group G2) of the “substituted or unsubstituted heterocyclic group” mentioned herein include unsubstituted heterocyclic groups (specific example group G2A) and substituted heterocyclic groups (specific example group G2B) below. (Herein, an unsubstituted heterocyclic group refers to an “unsubstituted heterocyclic group” in a “substituted or unsubstituted heterocyclic group,” and a substituted heterocyclic group refers to a “substituted heterocyclic group” in a “substituted or unsubstituted heterocyclic group.”) A simply termed “heterocyclic group” herein includes both of an “unsubstituted heterocyclic group” and a “substituted heterocyclic group.”

The “substituted heterocyclic group” refers to a group derived by substituting at least one hydrogen atom in an “unsubstituted heterocyclic group” with a substituent. Specific examples of the “substituted heterocyclic group” include a group derived by substituting at least one hydrogen atom in the “unsubstituted heterocyclic group” in the specific example group G2A below with a substituent, and examples of the substituted heterocyclic group in the specific example group G2B below. It should be noted that the examples of the “unsubstituted heterocyclic group” and the “substituted heterocyclic group” mentioned herein are merely exemplary, and the “substituted heterocyclic group” mentioned herein includes a group derived by further substituting a hydrogen atom bonded to a ring atom of a skeleton of a “substituted heterocyclic group” in the specific example group G2B below, and a group derived by further substituting a hydrogen atom of a substituent of the “substituted heterocyclic group” in the specific example group G2B below.

The specific example group G2A includes, for instance, unsubstituted heterocyclic groups including a nitrogen atom (specific example group G2A1) below, unsubstituted heterocyclic groups including an oxygen atom (specific example group G2A2) below, unsubstituted heterocyclic groups including a sulfur atom (specific example group G2A3) below, and monovalent heterocyclic groups (specific example group G2A4) derived by removing a hydrogen atom from cyclic structures represented by formulae (TEMP-16) to (TEMP-33) below.

The specific example group G2B includes, for instance, substituted heterocyclic groups including a nitrogen atom (specific example group G2B1) below, substituted heterocyclic groups including an oxygen atom (specific example group G2B2) below, substituted heterocyclic groups including a sulfur atom (specific example group G2B3) below, and groups derived by substituting at least one hydrogen atom of the monovalent heterocyclic groups (specific example group G2B4) derived from the cyclic structures represented by formulae (TEMP-16) to (TEMP-33) below.

Unsubstituted Heterocyclic Groups Including Nitrogen Atom (Specific Example Group G2A1): a pyrrolyl group, imidazolyl group, pyrazolyl group, triazolyl group, tetrazolyl group, oxazolyl group, isoxazolyl group, oxadiazolyl group, thiazolyl group, isothiazolyl group, thiadiazolyl group, pyridyl group, pyridazynyl group, pyrimidinyl group, pyrazinyl group, triazinyl group, indolyl group, isoindolyl group, indolizinyl group, quinolizinyl group, quinolyl group, isoquinolyl group, cinnolyl group, phthalazinyl group, quinazolinyl group, quinoxalinyl group, benzimidazolyl group, indazolyl group, phenanthrolinyl group, phenanthridinyl group, acridinyl group, phenazinyl group, carbazolyl group, benzocarbazolyl group, morpholino group, phenoxazinyl group, phenothiazinyl group, azacarbazolyl group, and diazacarbazolyl group.

Unsubstituted Heterocyclic Groups Including Oxygen Atom (Specific Example Group G2A2): a furyl group, oxazolyl group, isoxazolyl group, oxadiazolyl group, xanthenyl group, benzofuranyl group, isobenzofuranyl group, dibenzofuranyl group, naphthobenzofuranyl group, benzoxazolyl group, benzisoxazolyl group, phenoxazinyl group, morpholino group, dinaphthofuranyl group, azadibenzofuranyl group, diazadibenzofuranyl group, azanaphthobenzofuranyl group, and diazanaphthobenzofuranyl group.

Unsubstituted Heterocyclic Groups Including Sulfur Atom (Specific Example Group G2A3): a thienyl group, thiazolyl group, isothiazolyl group, thiadiazolyl group, benzothiophenyl group (benzothienyl group), isobenzothiophenyl group (isobenzothienyl group), dibenzothiophenyl group (dibenzothienyl group), naphthobenzothiophenyl group (nahthobenzothienyl group), benzothiazolyl group, benzisothiazolyl group, phenothiazinyl group, dinaphthothiophenyl group (dinaphthothienyl group), azadibenzothiophenyl group (azadibenzothienyl group), diazadibenzothiophenyl group (diazadibenzothienyl group), azanaphthobenzothiophenyl group (azanaphthobenzothienyl group), and diazanaphthobenzothiophenyl group (diazanaphthobenzothienyl group).

Monovalent Heterocyclic Groups Derived by Removing One Hydrogen Atom from Cyclic Structures Represented by Formulae (TEMP-16) to (TEMP-33) (Specific Example Group G2A4):

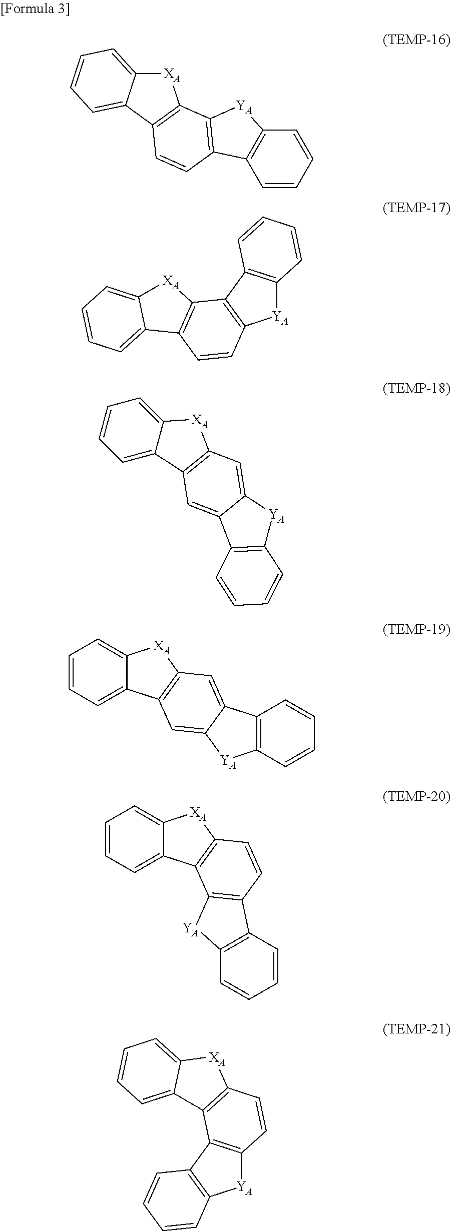

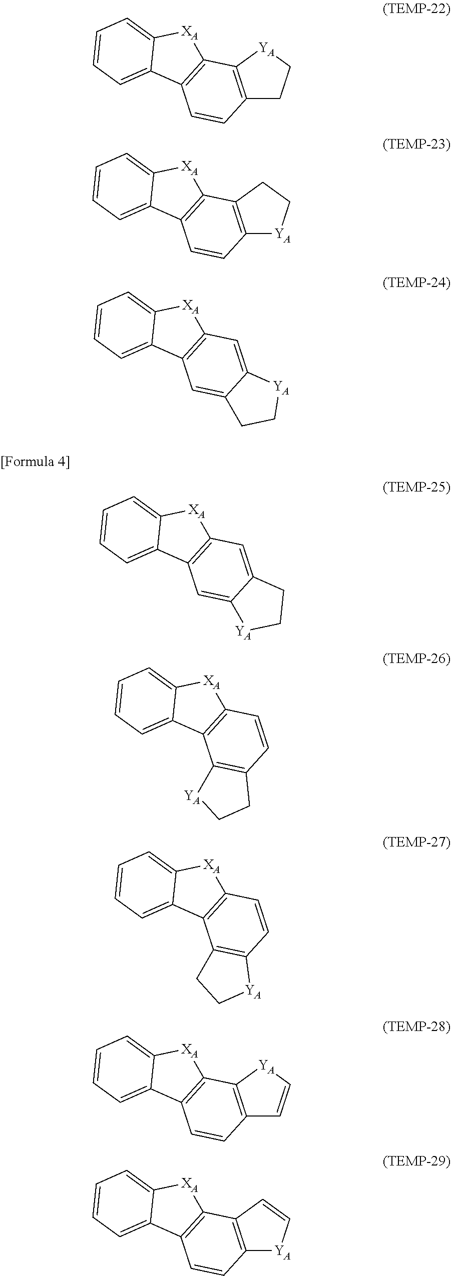

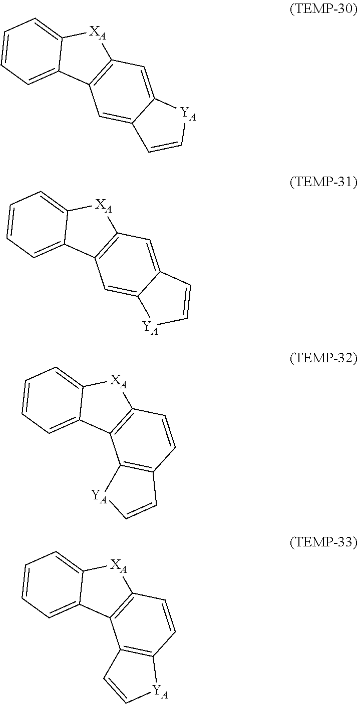

In the formulae (TEMP-16) to (TEMP-33), X_{A} and Y_{A} are each independently an oxygen atom, a sulfur atom, NH or CH₂, with a proviso that at least one of X_{A} or Y_{A} is an oxygen atom, a sulfur atom, or NH.

When at least one of X_{A} or Y_{A} in the formulae (TEMP-16) to (TEMP-33) is NH or CH₂, the monovalent heterocyclic groups derived from the cyclic structures represented by the formulae (TEMP-16) to (TEMP-33) include a monovalent group derived by removing one hydrogen atom from NH or CH₂.

Substituted Heterocyclic Groups Including Nitrogen Atom (Specific Example Group G2B1): a (9-phenyl) carbazolyl group, (9-biphenylyl) carbazolyl group, (9-phenyl)phenylcarbazolyl group, (9-naphthyl) carbazolyl group, diphenylcarbazole-9-yl group, phenylcarbazole-9-yl group, methylbenzimidazolyl group, ethylbenzimidazolyl group, phenyltriazinyl group, biphenylyltriazinyl group, diphenyltriazinyl group, phenylquinazolinyl group, and biphenylquinazolinyl group.

Substituted Heterocyclic Groups Including Oxygen Atom (Specific Example Group G2B2): a phenyldibenzofuranyl group, methyldibenzofuranyl group, t-butyldibenzofuranyl group, and monovalent residue of spiro[9H-xanthene-9,9′-[9H]fluorene].

Substituted Heterocyclic Groups Including Sulfur Atom (Specific Example Group G2B3): a phenyldibenzothiophenyl group, methyldibenzothiophenyl group, t-butyldibenzothiophenyl group, and monovalent residue of spiro[9H-thioxanthene-9,9′-[9H]fluorene].

Groups Obtained by Substituting at Least One Hydrogen Atom of Monovalent Heterocyclic Group Derived from Cyclic Structures Represented by Formulae (TEMP-16) to (TEMP-33) with Substituent (Specific Example Group G2B4):

The “at least one hydrogen atom of a monovalent heterocyclic group” means at least one hydrogen atom selected from a hydrogen atom bonded to a ring carbon atom of the monovalent heterocyclic group, a hydrogen atom bonded to a nitrogen atom of at least one of X_{A} or Y_{A} in a form of NH, and a hydrogen atom of one of X_{A} and Y_{A} in a form of a methylene group (CH₂).

### Substituted or Unsubstituted Alkyl Group

Specific examples (specific example group G3) of the “substituted or unsubstituted alkyl group” mentioned herein include unsubstituted alkyl groups (specific example group G3A) and substituted alkyl groups (specific example group G3B) below. (Herein, an unsubstituted alkyl group refers to an “unsubstituted alkyl group” in a “substituted or unsubstituted alkyl group,” and a substituted alkyl group refers to a “substituted alkyl group” in a “substituted or unsubstituted alkyl group.”) A simply termed “alkyl group” herein includes both of an “unsubstituted alkyl group” and a “substituted alkyl group”.

The “substituted alkyl group” refers to a group derived by substituting at least one hydrogen atom in an “unsubstituted alkyl group” with a substituent. Specific examples of the “substituted alkyl group” include a group derived by substituting at least one hydrogen atom of an “unsubstituted alkyl group” (specific example group G3A) below with a substituent, and examples of the substituted alkyl group (specific example group G3B) below. Herein, the alkyl group for the “unsubstituted alkyl group” refers to a chain alkyl group. Accordingly, the “unsubstituted alkyl group” include linear “unsubstituted alkyl group” and branched “unsubstituted alkyl group.” It should be noted that the examples of the “unsubstituted alkyl group” and the “substituted alkyl group” mentioned herein are merely exemplary, and the “substituted alkyl group” mentioned herein includes a group derived by further substituting a hydrogen atom of a skeleton of the “substituted alkyl group” in the specific example group G3B, and a group derived by further substituting a hydrogen atom of a substituent of the “substituted alkyl group” in the specific example group G3B.

Unsubstituted Alkyl Group (Specific Example Group G3A): a methyl group, ethyl group, n-propyl group, isopropyl group, n-butyl group, isobutyl group, s-butyl group, and t-butyl group.

Substituted Alkyl Group (Specific Example Group G3B): a heptafluoropropyl group (including isomer thereof), pentafluoroethyl group, 2,2,2-trifluoroethyl group, and trifluoromethyl group.

### Substituted or Unsubstituted Alkenyl Group

Specific examples (specific example group G4) of the “substituted or unsubstituted alkenyl group” mentioned herein include unsubstituted alkenyl groups (specific example group G4A) and substituted alkenyl groups (specific example group G4B). (Herein, an unsubstituted alkenyl group refers to an “unsubstituted alkenyl group” in a “substituted or unsubstituted alkenyl group,” and a substituted alkenyl group refers to a “substituted alkenyl group” in a “substituted or unsubstituted alkenyl group.”) A simply termed “alkenyl group” herein includes both of an “unsubstituted alkenyl group” and a “substituted alkenyl group”.

The “substituted alkenyl group” refers to a group derived by substituting at least one hydrogen atom in an “unsubstituted alkenyl group” with a substituent. Specific examples of the “substituted alkenyl group” include an “unsubstituted alkenyl group” (specific example group G4A) substituted by a substituent, and examples of the substituted alkenyl group (specific example group G4B) below. It should be noted that the examples of the “unsubstituted alkenyl group” and the “substituted alkenyl group” mentioned herein are merely exemplary, and the “substituted alkenyl group” mentioned herein includes a group derived by further substituting a hydrogen atom of a skeleton of the “substituted alkenyl group” in the specific example group G4B with a substituent, and a group derived by further substituting a hydrogen atom of a substituent of the “substituted alkenyl group” in the specific example group G4B with a substituent.

Unsubstituted Alkenyl Group (Specific Example Group G4A): a vinyl group, allyl group, 1-butenyl group, 2-butenyl group, and 3-butenyl group.

Substituted Alkenyl Group (Specific Example Group G4B): a 1,3-butanedienyl group, 1-methylvinyl group, 1-methylallyl group, 1,1-dimethylallyl group, 2-methylallyl group, and 1,2-dimethylallyl group.

### Substituted or Unsubstituted Alkynyl Group

Specific examples (specific example group G5) of the “substituted or unsubstituted alkynyl group” mentioned herein include unsubstituted alkynyl groups (specific example group G5A) below. (Herein, an unsubstituted alkynyl group refers to an “unsubstituted alkynyl group” in a “substituted or unsubstituted alkynyl group.”) A simply termed “alkynyl group” herein includes both of “unsubstituted alkynyl group” and “substituted alkynyl group”.

The “substituted alkynyl group” refers to a group derived by substituting at least one hydrogen atom in an “unsubstituted alkynyl group” with a substituent. Specific examples of the “substituted alkynyl group” include a group derived by substituting at least one hydrogen atom of the “unsubstituted alkynyl group” (specific example group G5A) below with a substituent.

Unsubstituted Alkynyl Group (Specific Example Group G5A): an ethynyl group.

### Substituted or Unsubstituted Cycloalkyl Group

Specific examples (specific example group G6) of the “substituted or unsubstituted cycloalkyl group” mentioned herein include unsubstituted cycloalkyl groups (specific example group G6A) and substituted cycloalkyl groups (specific example group G6B) below. (Herein, an unsubstituted cycloalkyl group refers to an “unsubstituted cycloalkyl group” in a “substituted or unsubstituted cycloalkyl group,” and a substituted cycloalkyl group refers to a “substituted cycloalkyl group” in a “substituted or unsubstituted cycloalkyl group.”) A simply termed “cycloalkyl group” herein includes both of “unsubstituted cycloalkyl group” and “substituted cycloalkyl group”.

The “substituted cycloalkyl group” refers to a group derived by substituting at least one hydrogen atom of an “unsubstituted cycloalkyl group” with a substituent. Specific examples of the “substituted cycloalkyl group” include a group derived by substituting at least one hydrogen atom of the “unsubstituted cycloalkyl group” (specific example group G6A) below with a substituent, and examples of the substituted cycloalkyl group (specific example group G6B) below. It should be noted that the examples of the “unsubstituted cycloalkyl group” and the “substituted cycloalkyl group” mentioned herein are merely exemplary, and the “substituted cycloalkyl group” mentioned herein includes a group derived by substituting at least one hydrogen atom bonded to a carbon atom of a skeleton of the “substituted cycloalkyl group” in the specific example group G6B with a substituent, and a group derived by further substituting a hydrogen atom of a substituent of the “substituted cycloalkyl group” in the specific example group G6B with a substituent.

Unsubstituted Cycloalkyl Group (Specific Example Group G6A): a cyclopropyl group, cyclobutyl group, cyclopentyl group, cyclohexyl group, 1-adamantyl group, 2-adamantyl group, 1-norbornyl group, and 2-norbornyl group.

Substituted Cycloalkyl Group (Specific Example Group G6B): a 4-methylcyclohexyl group.

Group Represented by —Si(R₉₀₁)(R₉₀₂)(R₉₀₃)

Specific examples (specific example group G7) of the group represented herein by —Si(R₉₀₁)(R₉₀₂)(R₉₀₃) include: —Si(G1)(G1)(G1); —Si (G1)(G2)(G2); —Si (G1)(G1)(G2); —Si (G2)(G2)(G2); —Si (G3)(G3)(G3); and —Si (G6)(G6)(G6);
where:
G1 represents a “substituted or unsubstituted aryl group” in the specific example group G1;
G2 represents a “substituted or unsubstituted heterocyclic group” in the specific example group G2;
G3 represents a “substituted or unsubstituted alkyl group” in the specific example group G3; and
G6 represents a “substituted or unsubstituted cycloalkyl group” in the specific example group G6;
a plurality of G1 in —Si (G1)(G1)(G1) are mutually the same or different;
a plurality of G2 in —Si (G1)(G2)(G2) are mutually the same or different;
a plurality of G1 in —Si (G1)(G1)(G2) are mutually the same or different;
a plurality of G2 in —Si (G2)(G2)(G2) are mutually the same or different;
a plurality of G3 in —Si (G3)(G3)(G3) are mutually the same or different; and
a plurality of G6 in —Si (G6)(G6)(G6) are mutually the same or different.

### Group Represented by —O—(R₉₀₄)

Specific examples (specific example group G8) of a group represented by —O—(R₉₀₄) herein include: —O(G1); —O(G2); —O(G3); and —O(G6);
where:
G1 represents a “substituted or unsubstituted aryl group” in the specific example group G1;
G2 represents a “substituted or unsubstituted heterocyclic group” in the specific example group G2;
G3 represents a “substituted or unsubstituted alkyl group” in the specific example group G3; and
G6 represents a “substituted or unsubstituted cycloalkyl group” in the specific example group G6.

### Group Represented by —S—(R₉₀₅)

Specific examples (specific example group G9) of a group represented herein by —S—(R₉₀₅) include: —S(G1); —S(G2); —S(G3); and —S(G6);
where:
G1 represents a “substituted or unsubstituted aryl group” in the specific example group G1;
G2 represents a “substituted or unsubstituted heterocyclic group” in the specific example group G2;
G3 represents a “substituted or unsubstituted alkyl group” in the specific example group G3; and
G6 represents a “substituted or unsubstituted cycloalkyl group” in the specific example group G6.
   Group Represented by —N(R₉₀₆)(R₉₀₇)

Specific examples (specific example group G10) of a group represented herein by —N(R₉₀₆)(R₉₀₇) include: —N(G1)(G1); —N(G2)(G2); —N(G1)(G2); —N(G3)(G3); and —N(G6)(G6);
where:
G1 represents a “substituted or unsubstituted aryl group” in the specific example group G1;
G2 represents a “substituted or unsubstituted heterocyclic group” in the specific example group G2;
G3 represents a “substituted or unsubstituted alkyl group” in the specific example group G3; and
G6 represents a “substituted or unsubstituted cycloalkyl group” in the specific example group G6;
a plurality of G1 in —N(G1)(G1) are mutually the same or different;
a plurality of G2 in —N(G2)(G2) are mutually the same or different;
a plurality of G3 in —N(G3)(G3) are mutually the same or different; and
a plurality of G6 in —N(G6)(G6) are mutually the same or different.

### Halogen Atom

Specific examples (specific example group G11) of “halogen atom” mentioned herein include a fluorine atom, chlorine atom, bromine atom, and iodine atom.

### Substituted or Unsubstituted Fluoroalkyl Group

The “substituted or unsubstituted fluoroalkyl group” mentioned herein refers to a group derived by substituting at least one hydrogen atom bonded to at least one of carbon atoms forming an alkyl group in the “substituted or unsubstituted alkyl group” with a fluorine atom, and also includes a group (perfluoro group) derived by substituting all of hydrogen atoms bonded to carbon atoms forming the alkyl group in the “substituted or unsubstituted alkyl group” with fluorine atoms. An “unsubstituted fluoroalkyl group” has, unless otherwise specified herein, 1 to 50, preferably 1 to 30, more preferably 1 to 18 carbon atoms. The “substituted fluoroalkyl group” refers to a group derived by substituting at least one hydrogen atom in a “fluoroalkyl group” with a substituent. It should be noted that the examples of the “substituted fluoroalkyl group” mentioned herein include a group derived by further substituting at least one hydrogen atom bonded to a carbon atom of an alkyl chain of a “substituted fluoroalkyl group” with a substituent, and a group derived by further substituting at least one hydrogen atom of a substituent of the “substituted fluoroalkyl group” with a substituent. Specific examples of the “unsubstituted fluoroalkyl group” include a group derived by substituting at least one hydrogen atom of the “alkyl group” (specific example group G3) with a fluorine atom.

### Substituted or Unsubstituted Haloalkyl Group

The “substituted or unsubstituted haloalkyl group” mentioned herein refers to a group derived by substituting at least one hydrogen atom bonded to carbon atoms forming the alkyl group in the “substituted or unsubstituted alkyl group” with a halogen atom, and also includes a group derived by substituting all hydrogen atoms bonded to carbon atoms forming the alkyl group in the “substituted or unsubstituted alkyl group” with halogen atoms. An “unsubstituted haloalkyl group” has, unless otherwise specified herein, 1 to 50, preferably 1 to 30, and more preferably 1 to 18 carbon atoms. The “substituted haloalkyl group” refers to a group derived by substituting at least one hydrogen atom in a “haloalkyl group” with a substituent. It should be noted that the examples of the “substituted haloalkyl group” mentioned herein include a group derived by further substituting at least one hydrogen atom bonded to a carbon atom of an alkyl chain of a “substituted haloalkyl group” with a substituent, and a group derived by further substituting at least one hydrogen atom of a substituent of the “substituted haloalkyl group” with a substituent. Specific examples of the “unsubstituted haloalkyl group” include a group derived by substituting at least one hydrogen atom of the “alkyl group” (specific example group G3) with a halogen atom. The haloalkyl group is occasionally referred to as a halogenated alkyl group.

### Substituted or Unsubstituted Alkoxy Group

Specific examples of a “substituted or unsubstituted alkoxy group” mentioned herein include a group represented by —O(G3), G3 being the “substituted or unsubstituted alkyl group” in the specific example group G3. An “unsubstituted alkoxy group” has, unless otherwise specified herein, 1 to 50, preferably 1 to 30, more preferably 1 to 18 carbon atoms.

### Substituted or Unsubstituted Alkylthio Group

Specific examples of a “substituted or unsubstituted alkylthio group” mentioned herein include a group represented by —S(G3), G3 being the “substituted or unsubstituted alkyl group” in the specific example group G3. An “unsubstituted alkylthio group” has, unless otherwise specified herein, 1 to 50, preferably 1 to 30, more preferably 1 to 18 carbon atoms.

### Substituted or Unsubstituted Aryloxy Group

Specific examples of a “substituted or unsubstituted aryloxy group” mentioned herein include a group represented by —O(G1), G1 being the “substituted or unsubstituted aryl group” in the specific example group G1. An “unsubstituted aryloxy group” has, unless otherwise specified herein, 6 to 50, preferably 6 to 30, more preferably 6 to 18 ring carbon atoms.

### Substituted or Unsubstituted Arylthio Group

Specific examples of a “substituted or unsubstituted arylthio group” mentioned herein include a group represented by —S(G1), G1 being the “substituted or unsubstituted aryl group” in the specific example group G1. An “unsubstituted arylthio group” has, unless otherwise specified herein, 6 to 50, preferably 6 to 30, more preferably 6 to 18 ring carbon atoms.

### Substituted or Unsubstituted Trialkylsilyl Group

Specific examples of a “trialkylsilyl group” mentioned herein include a group represented by —Si (G3)(G3)(G3), G3 being the “substituted or unsubstituted alkyl group” in the specific example group G3. The plurality of G3 in —Si (G3)(G3)(G3) are mutually the same or different. Each of the alkyl groups in the “trialkylsilyl group” has, unless otherwise specified herein, 1 to 50, preferably 1 to 20, more preferably 1 to 6 carbon atoms.

### Substituted or Unsubstituted Aralkyl Group

Specific examples of a “substituted or unsubstituted aralkyl group” mentioned herein include a group represented by -(G3)-(G1), G3 being the “substituted or unsubstituted alkyl group” in the specific example group G3, G1 being the “substituted or unsubstituted aryl group” in the specific example group G1. Accordingly, the “aralkyl group” is a group derived by substituting a hydrogen atom of the “alkyl group” with a substituent in a form of the “aryl group,” which is an example of the “substituted alkyl group.” An “unsubstituted aralkyl group,” which is an “unsubstituted alkyl group” substituted by an “unsubstituted aryl group,” has, unless otherwise specified herein, 7 to 50 carbon atoms, preferably 7 to 30 carbon atoms, more preferably 7 to 18 carbon atoms.

Specific examples of the “substituted or unsubstituted aralkyl group” include a benzyl group, 1-phenylethyl group, 2-phenylethyl group, 1-phenylisopropyl group, 2-phenylisopropyl group, phenyl-t-butyl group, a-naphthylmethyl group, 1-α-naphthylethyl group, 2-α-naphthylethyl group, 1-α-naphthylisopropyl group, 2-α-naphthylisopropyl group, β-naphthylmethyl group, 1-β-naphthylethyl group, 2-β-naphthylethyl group, 1-β-naphthylisopropyl group, and 2-β-naphthylisopropyl group.

Preferable examples of the substituted or unsubstituted aryl group mentioned herein include, unless otherwise specified herein, a phenyl group, p-biphenyl group, m-biphenyl group, o-biphenyl group, p-terphenyl-4-yl group, p-terphenyl-3-yl group, p-terphenyl-2-yl group, m-terphenyl-4-yl group, m-terphenyl-3-yl group, m-terphenyl-2-yl group, o-terphenyl-4-yl group, o-terphenyl-3-yl group, o-terphenyl-2-yl group, 1-naphthyl group, 2-naphthyl group, anthryl group, phenanthryl group, pyrenyl group, chrysenyl group, triphenylenyl group, fluorenyl group, 9,9′-spirobifluorenyl group, 9,9-dimethylfluorenyl group, and 9,9-diphenylfluorenyl group.

Preferable examples of the substituted or unsubstituted heterocyclic group mentioned herein include, unless otherwise specified herein, a pyridyl group, pyrimidinyl group, triazinyl group, quinolyl group, isoquinolyl group, quinazolinyl group, benzimidazolyl group, phenanthrolinyl group, carbazolyl group (1-carbazolyl group, 2-carbazolyl group, 3-carbazolyl group, 4-carbazolyl group, or 9-carbazolyl group), benzocarbazolyl group, azacarbazolyl group, diazacarbazolyl group, dibenzofuranyl group, naphthobenzofuranyl group, azadibenzofuranyl group, diazadibenzofuranyl group, dibenzothiophenyl group, naphthobenzothiophenyl group, azadibenzothiophenyl group, diazadibenzothiophenyl group, (9-phenyl) carbazolyl group ((9-phenyl) carbazole-1-yl group, (9-phenyl) carbazole-2-yl group, (9-phenyl) carbazole-3-yl group, or (9-phenyl) carbazole-4-yl group), (9-biphenylyl) carbazolyl group, (9-phenyl)phenylcarbazolyl group, diphenylcarbazole-9-yl group, phenylcarbazole-9-yl group, phenyltriazinyl group, biphenylyltriazinyl group, diphenyltriazinyl group, phenyldibenzofuranyl group, and phenyldibenzothiophenyl group.

The carbazolyl group mentioned herein is, unless otherwise specified herein, specifically a group represented by one of formulae below.

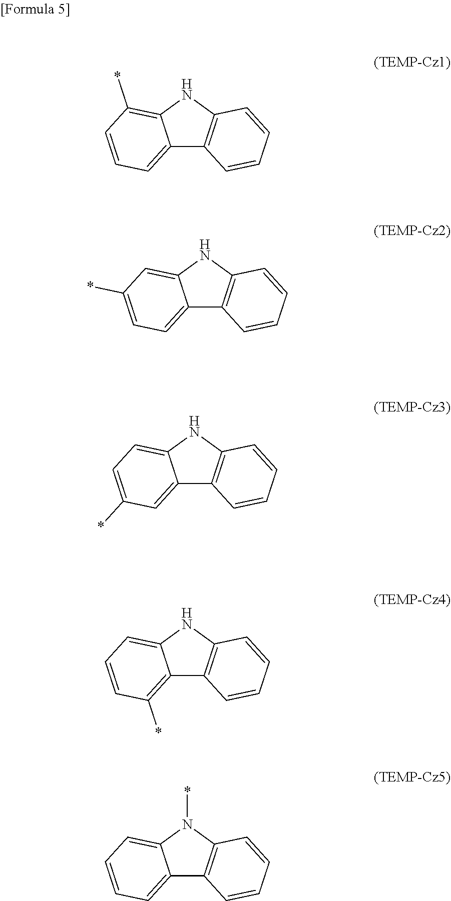 [SMILES: *c1cccc2c1[nH]c1ccccc12 ; *c1ccc2c(c1)[nH]c1ccccc12 ; *c1ccc2[nH]c3ccccc3c2c1 ; *c1cccc2[nH]c3ccccc3c12 ; *n1c2ccccc2c2ccccc21]

The (9-phenyl) carbazolyl group mentioned herein is, unless otherwise specified herein, specifically a group represented by one of formulae below.

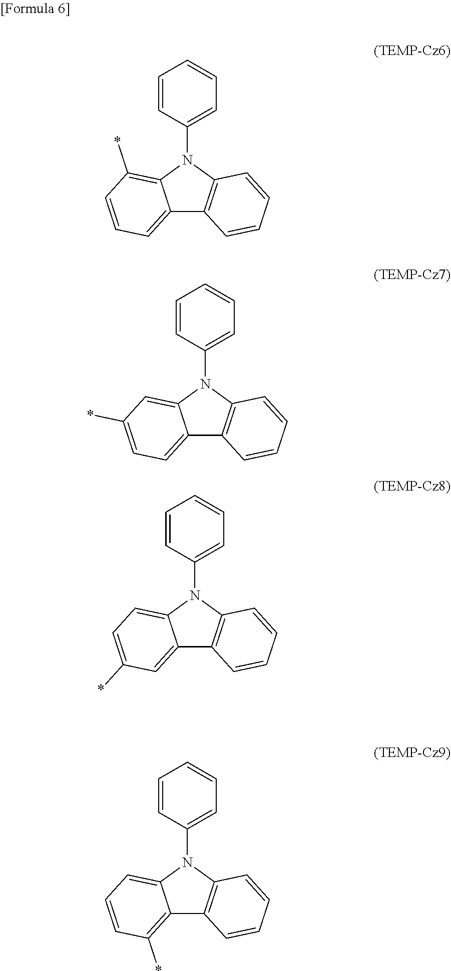

In the formulae (TEMP-Cz1) to (TEMP-Cz9), * represents a bonding position.

The dibenzofuranyl group and dibenzothiophenyl group mentioned herein are, unless otherwise specified herein, each specifically represented by one of formulae below.

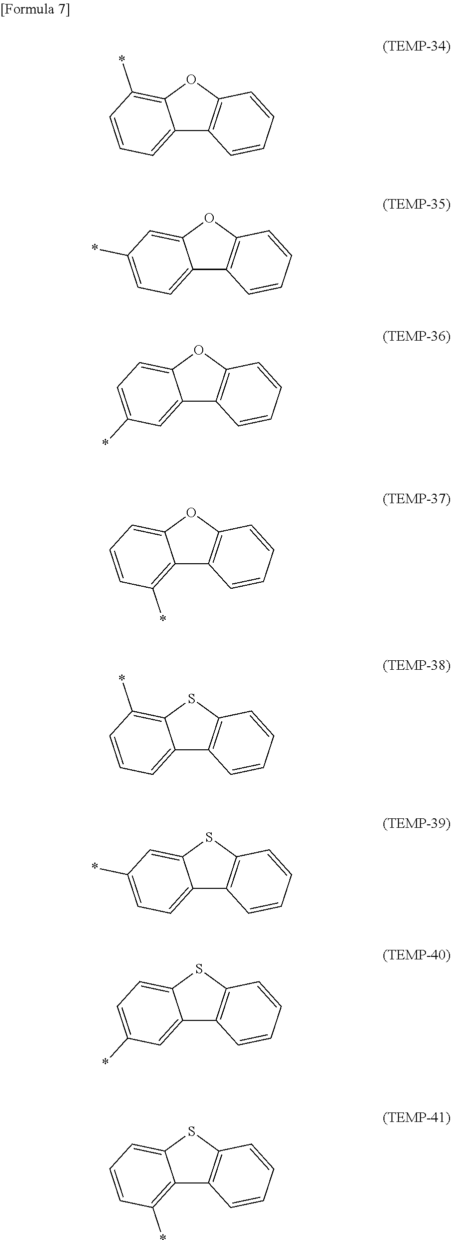

In the formulae (TEMP-34) to (TEMP-41), * represents a bonding position.

Preferable examples of the substituted or unsubstituted alkyl group mentioned herein include, unless otherwise specified herein, a methyl group, ethyl group, propyl group, isopropyl group, n-butyl group, isobutyl group, and t-butyl group.

### Substituted or Unsubstituted Arylene Group

The “substituted or unsubstituted arylene group” mentioned herein is, unless otherwise specified herein, a divalent group derived by removing one hydrogen atom on an aryl ring of the “substituted or unsubstituted aryl group.” Specific examples of the “substituted or unsubstituted arylene group” (specific example group G12) include a divalent group derived by removing one hydrogen atom on an aryl ring of the “substituted or unsubstituted aryl group” in the specific example group G1.

### Substituted or Unsubstituted Divalent Heterocyclic Group

The “substituted or unsubstituted divalent heterocyclic group” mentioned herein is, unless otherwise specified herein, a divalent group derived by removing one hydrogen atom on a heterocycle of the “substituted or unsubstituted heterocyclic group.” Specific examples of the “substituted or unsubstituted divalent heterocyclic group” (specific example group G13) include a divalent group derived by removing one hydrogen atom on a heterocyclic ring of the “substituted or unsubstituted heterocyclic group” in the specific example group G2.

### Substituted or Unsubstituted Alkylene Group

The “substituted or unsubstituted alkylene group” mentioned herein is, unless otherwise specified herein, a divalent group derived by removing one hydrogen atom on an alkyl chain of the “substituted or unsubstituted alkyl group.” Specific examples of the “substituted or unsubstituted alkylene group” (specific example group G14) include a divalent group derived by removing one hydrogen atom on an alkyl chain of the “substituted or unsubstituted alkyl group” in the specific example group G3.

The substituted or unsubstituted arylene group mentioned herein is, unless otherwise specified herein, preferably any one of groups represented by formulae (TEMP-42) to (TEMP-68) below.

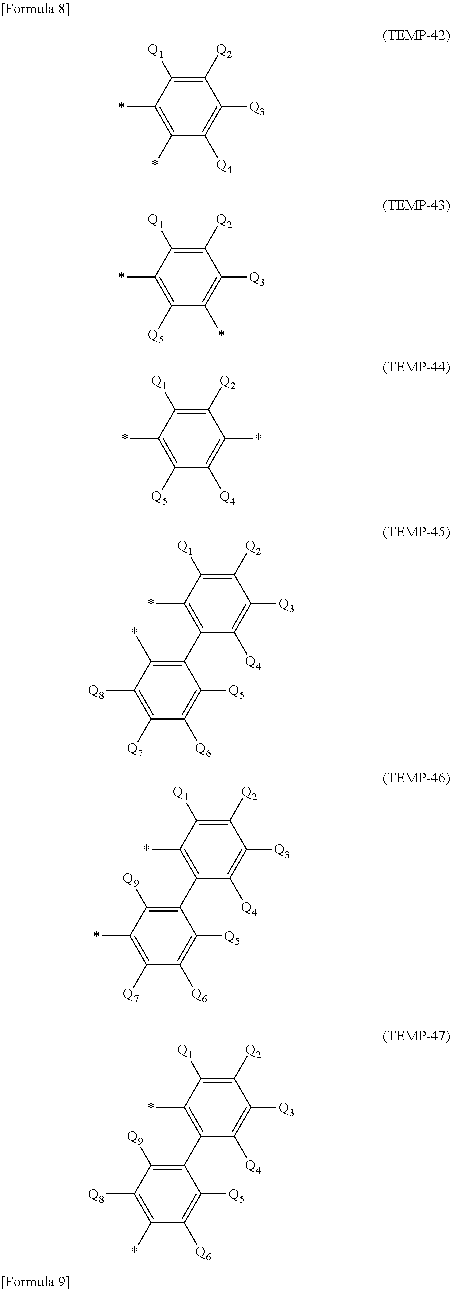

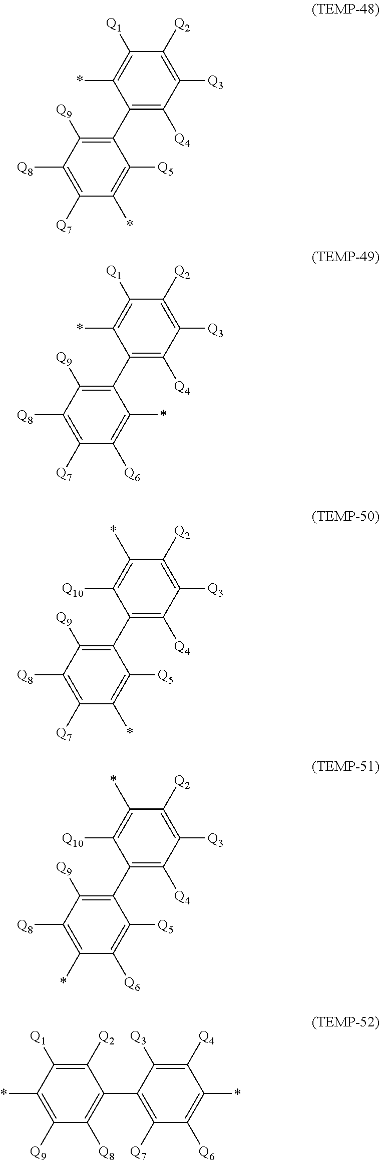

In the formulae (TEMP-42) to (TEMP-52), Q₁ to Q₁₀ are each independently a hydrogen atom or a substituent.

In the formulae (TEMP-42) to (TEMP-52), * represents a bonding position.

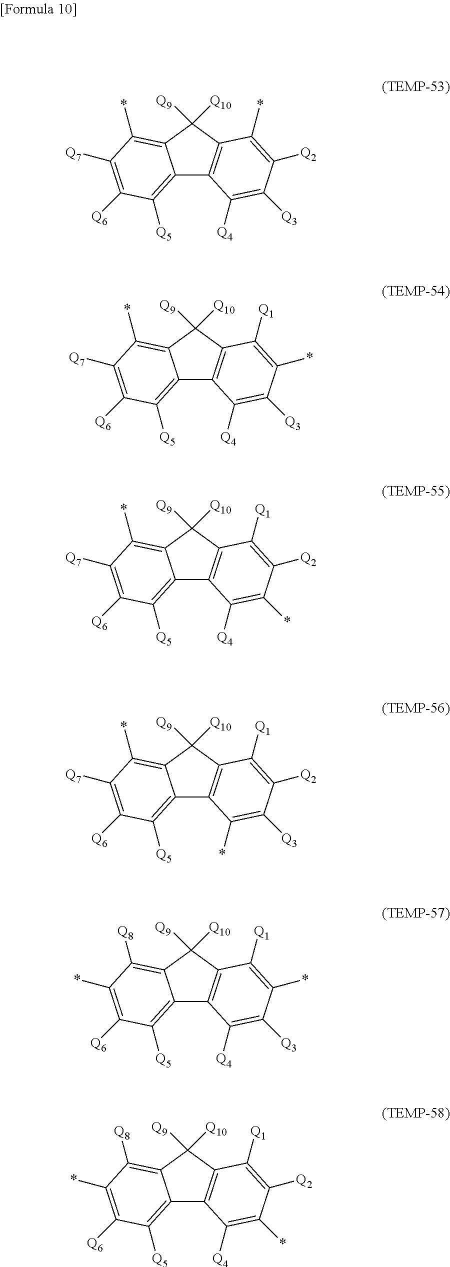

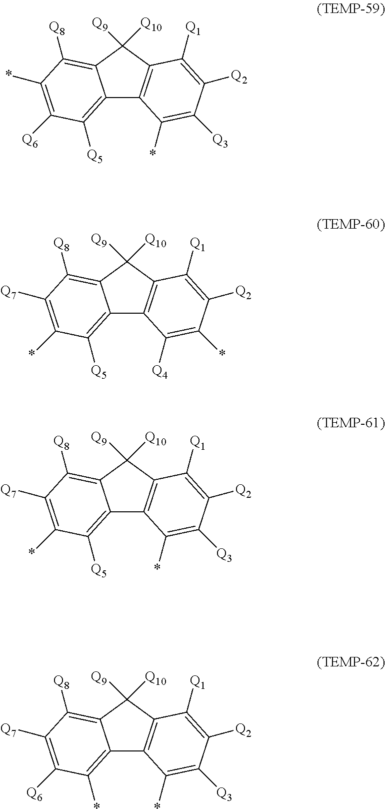

In the formulae (TEMP-53) to (TEMP-62), Q₁ to Q₁₀ are each independently a hydrogen atom or a substituent.

In the formulae, Q₉ and Q₁₀ may be mutually bonded through a single bond to form a ring.

In the formulae (TEMP-53) to (TEMP-62), * represents a bonding position.

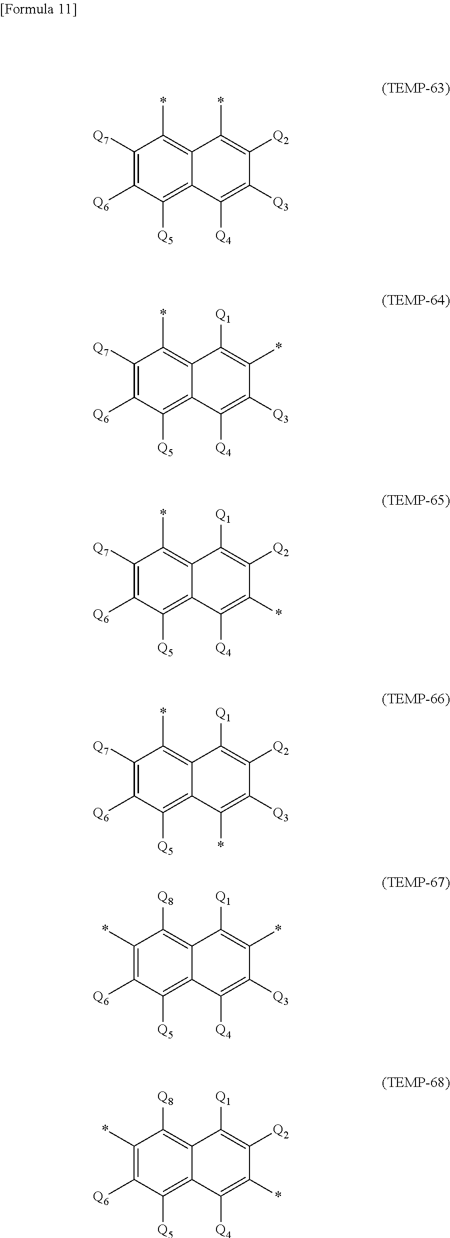

In the formulae (TEMP-63) to (TEMP-68), Q₁ to Q₈ are each independently a hydrogen atom or a substituent.

In the formulae (TEMP-63) to (TEMP-68), * represents a bonding position.

The substituted or unsubstituted divalent heterocyclic group mentioned herein is, unless otherwise specified herein, preferably a group represented by any one of formulae (TEMP-69) to (TEMP-102) below.

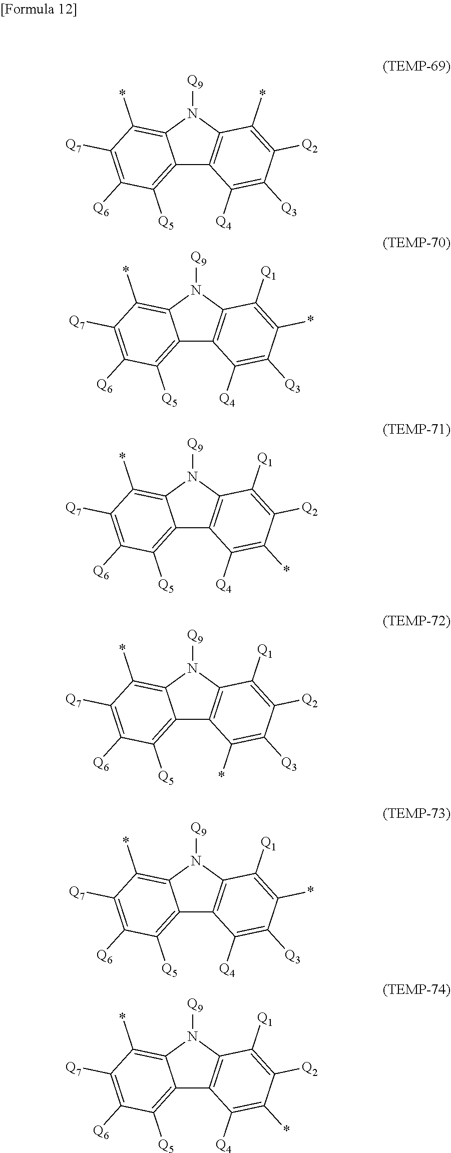

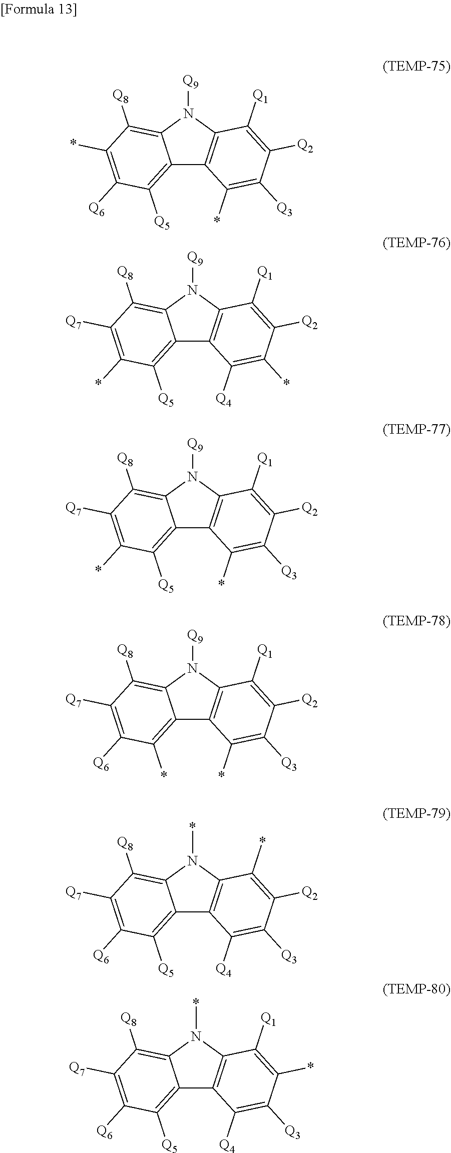

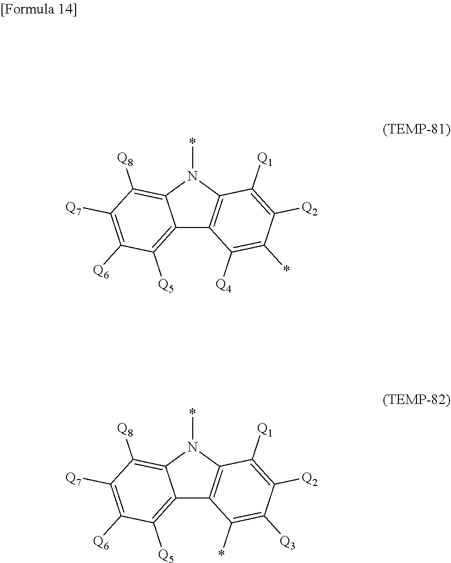

In the formulae (TEMP-69) to (TEMP-82), Q₁ to Q₉ are each independently a hydrogen atom or a substituent.

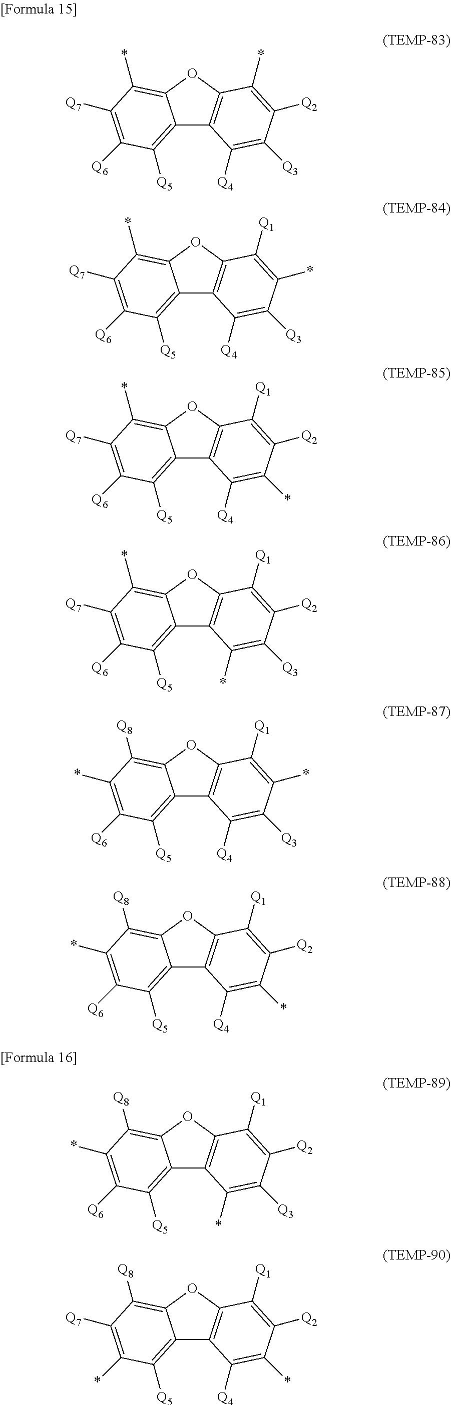

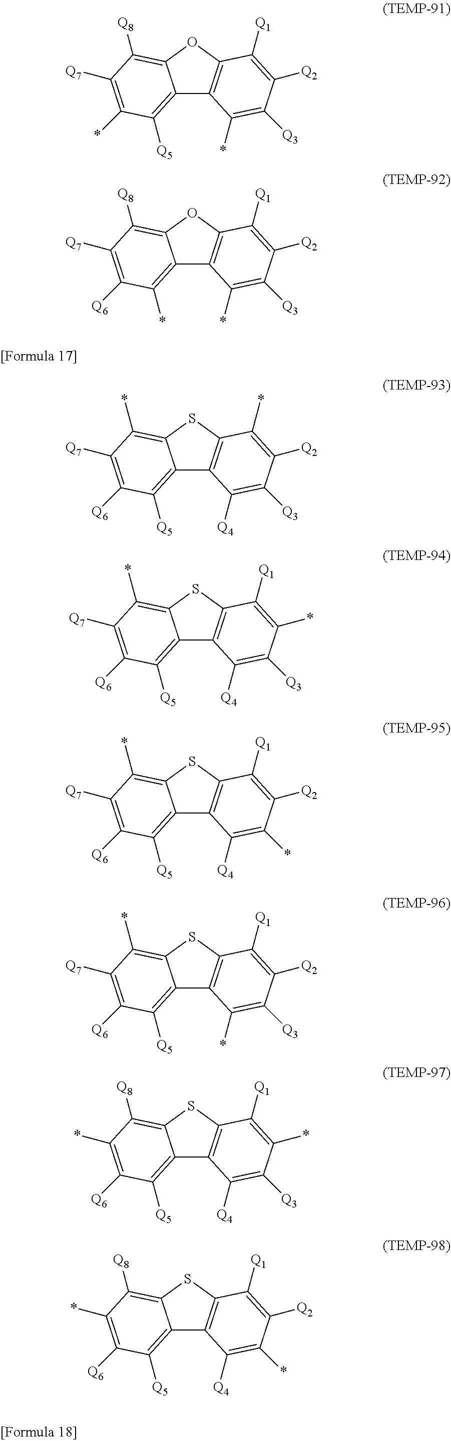

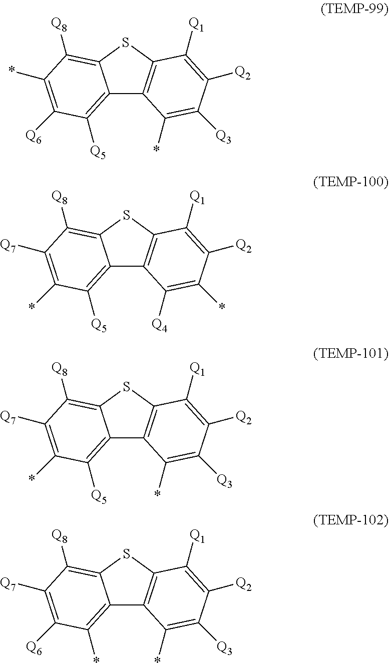

In the formulae (TEMP-83) to (TEMP-102), Q₁ to Q₈ are each independently a hydrogen atom or a substituent.

The substituent mentioned herein has been described above.

Instance of “Bonded to Form Ring” Instances where “at least one combination of adjacent two or more (of . . . ) are mutually bonded to form a substituted or unsubstituted monocyclic ring, mutually bonded to form a substituted or unsubstituted fused ring, or not mutually bonded” mentioned herein refer to instances where “at least one combination of adjacent two or more (of . . . ) are mutually bonded to form a substituted or unsubstituted monocyclic ring”, “at least one combination of adjacent two or more (of . . . ) are mutually bonded to form a substituted or unsubstituted fused ring,” and “at least one combination of adjacent two or more (of . . . ) are not mutually bonded.”

Instances where “at least one combination of adjacent two or more (of . . . ) are mutually bonded to form a substituted or unsubstituted monocyclic ring” and “at least one combination of adjacent two or more (of . . . ) are mutually bonded to form a substituted or unsubstituted fused ring” mentioned herein (these instances will be sometimes collectively referred to as an instance of “bonded to form a ring” hereinafter) will be described below. An anthracene compound having a basic skeleton in a form of an anthracene ring and represented by a formula (TEMP-103) below will be used as an example for the description.

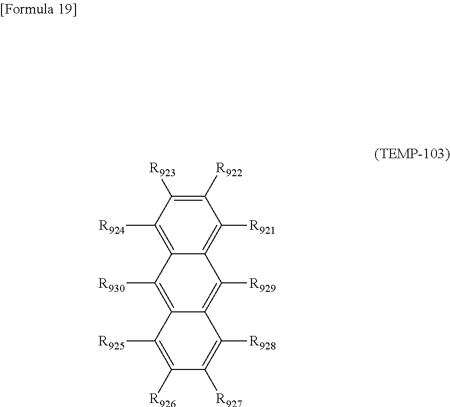

For instance, when “at least one combination of adjacent two or more of R₉₂₁ to R₉₃₀ are mutually bonded to form a ring,” the combination of adjacent ones of R₉₂₁ to R₉₃₀ (i.e. the combination at issue) is a combination of R₉₂₁ and R₉₂₂, a combination of R₉₂₂ and R₉₂₃, a combination of R₉₂₃ and R₉₂₄, a combination of R₉₂₄ and R₉₃₀, a combination of R₉₃₀ and R₉₂₅, a combination of R₉₂₅ and R₉₂₆, a combination of R₉₂₆ and R₉₂₇, a combination of R₉₂₇ and R₉₂₈, a combination of R₉₂₈ and R₉₂₉, or a combination of R₉₂₉ and R₉₂₁.

The term “at least one combination” means that two or more of the above combinations of adjacent two or more of R₉₂₁ to R₉₃₀ may simultaneously form rings. For instance, when R₉₂₁ and R₉₂₂ are mutually bonded to form a ring Q_{A} and R₉₂₅ and R₉₂₆ are simultaneously mutually bonded to form a ring Q_{B}, the anthracene compound represented by the formula (TEMP-103) is represented by a formula (TEMP-104) below.

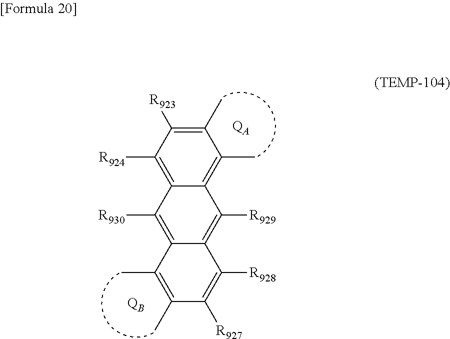

The instance where the “combination of adjacent two or more” form a ring means not only an instance where the “two” adjacent components are bonded but also an instance where adjacent “three or more” are bonded. For instance, R₉₂₁ and R₉₂₂ are mutually bonded to form a ring Q_{A} and R₉₂₂ and R₉₂₃ are mutually bonded to form a ring Qc, and mutually adjacent three components (R₉₂₁, R₉₂₂ and R₉₂₃) are mutually bonded to form a ring fused to the anthracene basic skeleton. In this case, the anthracene compound represented by the formula (TEMP-103) is represented by a formula (TEMP-105) below. In the formula (TEMP-105) below, the ring Q_{A} and the ring Qc share R₉₂₂.

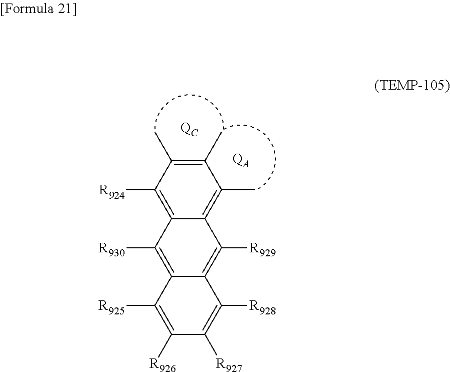

The formed “monocyclic ring” or “fused ring” may be, in terms of the formed ring in itself, a saturated ring or an unsaturated ring. When the “combination of adjacent two” form a “monocyclic ring” or a “fused ring,” the “monocyclic ring” or “fused ring” may be a saturated ring or an unsaturated ring. For instance, the ring Q_{A} and the ring Q_{B} formed in the formula (TEMP-104) are each independently a “monocyclic ring” or a “fused ring.” Further, the ring Q_{A} and the ring Qc formed in the formula (TEMP-105) are each a “fused ring.” The ring Q_{A} and the ring Qc in the formula (TEMP-105) are fused to form a fused ring. When the ring Q_{A} in the formula (TEMP-104) is a benzene ring, the ring Q_{A} is a monocyclic ring. When the ring Q_{A} in the formula (TEMP-104) is a naphthalene ring, the ring Q_{A} is a fused ring.

The “unsaturated ring” represents an aromatic hydrocarbon ring or an aromatic heterocycle. The “saturated ring” represents an aliphatic hydrocarbon ring or a non-aromatic heterocycle.

Specific examples of the aromatic hydrocarbon ring include a ring formed by terminating a bond of a group in the specific example of the specific example group G1 with a hydrogen atom.

Specific examples of the aromatic heterocycle include a ring formed by terminating a bond of an aromatic heterocyclic group in the specific example of the specific example group G2 with a hydrogen atom.

Specific examples of the aliphatic hydrocarbon ring include a ring formed by terminating a bond of a group in the specific example of the specific example group G6 with a hydrogen atom.

The phrase “to form a ring” herein means that a ring is formed only by a plurality of atoms of a basic skeleton, or by a combination of a plurality of atoms of the basic skeleton and one or more optional atoms. For instance, the ring Q_{A} formed by mutually bonding R₉₂₁ and R₉₂₂ shown in the formula (TEMP-104) is a ring formed by a carbon atom of the anthracene skeleton bonded to R₉₂₁, a carbon atom of the anthracene skeleton bonded to R₉₂₂, and one or more optional atoms. Specifically, when the ring Q_{A} is a monocyclic unsaturated ring formed by R₉₂₁ and R₉₂₂, the ring formed by a carbon atom of the anthracene skeleton bonded to R₉₂₁, a carbon atom of the anthracene skeleton bonded to R₉₂₂, and four carbon atoms is a benzene ring.

The “optional atom” is, unless otherwise specified herein, preferably at least one atom selected from the group consisting of a carbon atom, nitrogen atom, oxygen atom, and sulfur atom. A bond of the optional atom (e.g. a carbon atom and a nitrogen atom) not forming a ring may be terminated by a hydrogen atom or the like or may be substituted by an “optional substituent” described later. When the ring includes any other optional element than the carbon atom, the resultant ring is a heterocycle.

The number of “one or more optional atoms” forming the monocyclic ring or fused ring is, unless otherwise specified herein, preferably in a range from 2 to 15, more preferably in a range from 3 to 12, further preferably in a range from 3 to 5.

Unless otherwise specified herein, the ring, which may be a “monocyclic ring” or “fused ring,” is preferably a “monocyclic ring.”

Unless otherwise specified herein, the ring, which may be a “saturated ring” or “unsaturated ring,” is preferably an “unsaturated ring.”

Unless otherwise specified herein, the “monocyclic ring” is preferably a benzene ring.

Unless otherwise specified herein, the “unsaturated ring” is preferably a benzene ring.

When “at least one combination of adjacent two or more” (of . . . ) are “mutually bonded to form a substituted or unsubstituted monocyclic ring” or “mutually bonded to form a substituted or unsubstituted fused ring,” unless otherwise specified herein, at least one combination of adjacent two or more of components are preferably mutually bonded to form a substituted or unsubstituted “unsaturated ring” formed of a plurality of atoms of the basic skeleton, and 1 to 15 atoms of at least one element selected from the group consisting of carbon, nitrogen, oxygen and sulfur.

When the “monocyclic ring” or the “fused ring” has a substituent, the substituent is the substituent described in later-described “optional substituent.” When the “monocyclic ring” or the “fused ring” has a substituent, specific examples of the substituent are the substituents described in the above under the subtitle “Substituent Mentioned Herein.”

When the “saturated ring” or the “unsaturated ring” has a substituent, the substituent is the substituent described in later-described “optional substituent.” When the “monocyclic ring” or the “fused ring” has a substituent, specific examples of the substituent are the substituents described in the above under the subtitle “Substituent Mentioned Herein.”

The above is the description for the instances where “at least one combination of adjacent two or more (of . . . ) are mutually bonded to form a substituted or unsubstituted monocyclic ring” and “at least one combination of adjacent two or more (of . . . ) are mutually bonded to form a substituted or unsubstituted fused ring” mentioned herein (sometimes referred to as an instance of “bonded to form a ring”).

### Substituent for Substituted or Unsubstituted Group

In an exemplary embodiment herein, the substituent for the substituted or unsubstituted group (hereinafter occasionally referred to as an “optional substituent”), is for instance, a group selected from the group consisting of an unsubstituted alkyl group having 1 to 50 carbon atoms, an unsubstituted alkenyl group having 2 to 50 carbon atoms, an unsubstituted alkynyl group having 2 to 50 carbon atoms, an unsubstituted cycloalkyl group having 3 to 50 ring carbon atoms, —Si(R₉₀₁)(R₉₀₂)(R₉₀₃), —O—(R₉₀₄), —S—(R₉₀₅), —N(R₉₀₆)(R₉₀₇), a halogen atom, a cyano group, a nitro group, an unsubstituted aryl group having 6 to 50 ring carbon atoms, and an unsubstituted heterocyclic group having 5 to 50 ring atoms;

R₉₀₁ to R₉₀₇ are each independently a hydrogen atom, a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms;
when two or more R₉₀₁ are present, the two or more R₉₀₁ are mutually the same or different;
when two or more R₉₀₂ are present, the two or more R₉₀₂ are mutually the same or different;
when two or more R₉₀₃ are present, the two or more R₉₀₃ are mutually the same or different;
when two or more R₉₀₄ are present, the two or more R₉₀₄ are mutually the same or different;
when two or more R₉₀₅ are present, the two or more R₉₀₅ are mutually the same or different;
when two or more R₉₀₆ are present, the two or more R₉₀₆ are mutually the same or different; and
when two or more R₉₀₇ are present, the two or more R₉₀₇ are mutually the same or different.

In an exemplary embodiment, the substituent for the substituted or unsubstituted group is a group selected from the group consisting of an alkyl group having 1 to 50 carbon atoms, an aryl group having 6 to 50 ring carbon atoms, and a heterocyclic group having 5 to 50 ring atoms.

In an exemplary embodiment, the substituent for the substituted or unsubstituted group is a group selected from the group consisting of an alkyl group having 1 to 18 carbon atoms, an aryl group having 6 to 18 ring carbon atoms, and a heterocyclic group having 5 to 18 ring atoms.

Specific examples of the above optional substituent are the same as the specific examples of the substituent described in the above under the subtitle “Substituent Mentioned Herein.”

Unless otherwise specified herein, adjacent ones of the optional substituents may form a “saturated ring” or an “unsaturated ring,” preferably a substituted or unsubstituted saturated five-membered ring, a substituted or unsubstituted saturated six-membered ring, a substituted or unsubstituted unsaturated five-membered ring, or a substituted or unsubstituted unsaturated six-membered ring, more preferably a benzene ring.

Unless otherwise specified herein, the optional substituent may further include a substituent. Examples of the substituent for the optional substituent are the same as the examples of the optional substituent.

Herein, numerical ranges represented by “AA to BB” represent a range whose lower limit is the value (AA) recited before “to” and whose upper limit is the value (BB) recited after “to.”

Herein, a numerical formula represented by “A≥B” means that the value A is equal to the value B, or the value A is larger than the value B.

Herein, a numerical formula represented by “A≤B” means that the value A is equal to the value B, or the value A is smaller than the value B.

### First Exemplary Embodiment

### Organic Electroluminescence Device

An organic electroluminescence device of a first exemplary embodiment includes: an anode; a cathode; an emitting region disposed between the anode and the cathode; and a first anode side organic layer disposed between the emitting region and the anode, in which: the first anode side organic layer contains a first material; the emitting region includes a first emitting layer and a second emitting layer; the first emitting layer is disposed between the first anode side organic layer and the second emitting layer; the first emitting layer is in direct contact with the first anode side organic layer; the first emitting layer contains a first host material and a second host material; the second emitting layer contains a third host material; an energy level of a highest occupied molecular orbital of the first material HOMO(HT1), an energy level of a highest occupied molecular orbital of the first host material HOMO(H1), and an energy level of a highest occupied molecular orbital of the second host material HOMO(H2) satisfy a relationship of a numerical formula (Numerical Formula A1) below; a triplet energy of the first host material T₁(H1) and a triplet energy of the third host material T₁(H3) satisfy a relationship of a numerical formula (Numerical Formula A2) below; and a triplet energy of the second host material T₁(H2) and the triplet energy of the third host material T₁(H3) satisfy a relationship of a numerical formula (Numerical Formula A3) below.

$$HOMO ( HT 1 ) > HOMO ( H 2 ) > HOMO ( H 1 ) \qquad ( Numerical Formula A1 )$$

$${T}_{1} ( H 1 ) > {T}_{1} ( H 3 ) \qquad ( Numerical Formula A2 )$$

$${T}_{1} ( H 2 ) > {T}_{1} ( H 3 ) . \qquad ( Numerical Formula A3 )$$

According to the exemplary embodiment, there can be provided an organic electroluminescence device capable of maintaining luminous efficiency and having a long lifetime.

In the organic electroluminescence device according to the exemplary embodiment, the first emitting layer contains two host materials, which are a compound of which energy level of a highest occupied molecular orbital HOMO is large and a compound of which energy level of a highest occupied molecular orbital HOMO is small, and satisfies the relationship of the numerical formula (Numerical Formula A1). Compared to an organic EL device including an emitting layer that contains a single host material, the organic EL device of the exemplary embodiment with improved hole injectability to the first emitting layer maintains luminous efficiency and extends a lifetime thereof.

In the organic electroluminescence device according to the exemplary embodiment, the emitting region includes the first emitting layer and the second emitting layer. The luminous efficiency of the organic electroluminescence device is thus improved.

Conventionally, triplet-triplet-annihilation (occasionally referred to as TTA) has been known as a technique for improving the luminous efficiency of the organic electroluminescence device. TTA is a mechanism in which triplet excitons collide with one another to generate singlet excitons. The TTA mechanism is also referred to as a TTF mechanism as described in Patent Literature 2.

The TTF phenomenon will be described. Holes injected from an anode and electrons injected from a cathode are recombined in an emitting layer to generate excitons. As for the spin state, as is conventionally known, singlet excitons account for 25% and triplet excitons account for 75%. In a conventionally known fluorescent device, light is emitted when singlet excitons of 25% are relaxed to the ground state. The remaining triplet excitons of 75% are returned to the ground state without emitting light through a thermal deactivation process. Accordingly, the theoretical limit value of the internal quantum efficiency of the conventional fluorescent device is believed to be 25%.

The behavior of triplet excitons generated within an organic substance has been theoretically examined. According to S. M. Bachilo et al. (J. Phys. Chem. A, 104, 7711 (2000)), assuming that high-order excitons such as quintet excitons are quickly returned to triplet excitons, triplet excitons (hereinafter abbreviated as 3A*) collide with one another with an increase in density thereof, whereby a reaction shown by the following formula occurs. In the formula, 1A represents the ground state and 1A* represents the lowest singlet excitons.

$${}^{3} {A}^{*} + {}^{3} {A}^{*} \to ( 4 / 9 ) {}^{1} A + ( 1 / 9 ) {}^{1} {A}^{*} + ( 13 / 9 ) {}^{3} {A}^{*}$$

In other words, 5³A*→4¹A+1A* is satisfied, and it is expected that, among triplet excitons initially generated, which account for 75%, one fifth thereof (i.e., 20%) is changed to singlet excitons. Accordingly, the amount of singlet excitons which contribute to emission is 40%, which is a value obtained by adding 15% (75%×(⅕)=15%) to 25%, which is the amount ratio of initially generated singlet excitons. At this time, a ratio of luminous intensity derived from TTF (TTF ratio) relative to the total luminous intensity is 15/40, i.e., 37.5%. Assuming that singlet excitons are generated by collision of initially generated triplet excitons accounting for 75% (i.e., one singlet exciton is generated from two triplet excitons), a significantly high internal quantum efficiency of 62.5% is obtained, which is a value obtained by adding 37.5% (75%×(½)=37.5%) to 25% (the amount ratio of initially generated singlet excitons). At this time, the TTF ratio is 37.5/62.5=60%.

In the organic electroluminescence device according to the exemplary embodiment, it is considered that triplet excitons generated by recombination of holes and electrons in the first emitting layer and present on an interface between the first emitting layer and organic layer(s) in direct contact therewith are not likely to be quenched even under the presence of excessive carriers on the interface between the first emitting layer and the organic layer(s). For instance, the presence of a recombination region locally on an interface between the first emitting layer and a hole transporting layer or an electron blocking layer is considered to cause quenching by excessive electrons. Meanwhile, the presence of a recombination region locally on an interface between the first emitting layer and an electron transporting layer or a hole blocking layer is considered to cause quenching by excessive holes.

The organic electroluminescence device according to the exemplary embodiment includes at least two emitting layers (i.e., the first emitting layer and the second emitting layer) satisfying a predetermined relationship. The triplet energy of the first host material T₁(H1) in the first emitting layer and the triplet energy of the third host material T₁(H3) in the second emitting layer satisfy the relationship of the numerical formula (Numerical Formula A2), and the triplet energy of the second host material T₁(H2) in the first emitting layer and the triplet energy of the third host material T₁(H3) in the second emitting layer satisfy the relationship of the numerical formula (Numerical Formula A3).

By including the first emitting layer and the second emitting layer so as to satisfy the relationships of the numerical formulae (Numerical Formula A2 and Numerical Formula A3), triplet excitons generated in the first emitting layer can transfer to the second emitting layer without being quenched by excessive carriers and be inhibited from back-transferring from the second emitting layer to the first emitting layer. Consequently, the second emitting layer exhibits the TTF mechanism to effectively generate singlet excitons, thereby improving the luminous efficiency.

Accordingly, the organic electroluminescence device includes, as different regions, the first emitting layer mainly generating triplet excitons and the second emitting layer mainly exhibiting the TTF mechanism using triplet excitons having transferred from the first emitting layer, and has a difference in triplet energy provided by using a compound having a smaller triplet energy than that of the first host material and the second host material in the first emitting layer as the third host material in the second emitting layer. The luminous efficiency is thus improved.

In an exemplary arrangement of the organic EL device according to the exemplary embodiment, the triplet energy of the first host material T₁(H1) satisfies a relationship of a numerical formula (Numerical Formula A4) below, and the triplet energy of the second host material T₁(H2) satisfies a relationship of a numerical formula (Numerical Formula A5) below.

$$1.8 eV < {T}_{1} ( H 1 ) < 2.5 eV \qquad ( Numerical Formula A4 )$$

$$1.8 eV < {T}_{1} ( H 2 ) < 2.5 eV \qquad ( Numerical Formula A5 )$$

In an exemplary arrangement of the organic EL device according to the exemplary embodiment, the triplet energy of the first host material T₁(H1) satisfies a relationship of a numerical formula (Numerical Formula A41) below, and the triplet energy of the second host material T₁(H2) satisfies a relationship of a numerical formula (Numerical Formula A51) below.

$$2. eV < {T}_{1} ( H 1 ) < 2.3 eV \qquad ( Numerical Formula A41 )$$

$$2. eV < {T}_{1} ( H 2 ) < 2.3 eV \qquad ( Numerical Formula A51 )$$

In an exemplary arrangement of the organic EL device according to the exemplary embodiment, the triplet energy of the first host material T₁(H1) and the triplet energy of the third host material T₁(H3) satisfy a relationship of a numerical formula (Numerical Formula A21) below.

$${T}_{1} ( H 1 ) - {T}_{1} ( H 3 ) > 0.03 eV \qquad ( Numerical Formula A21 )$$

In an exemplary arrangement of the organic EL device according to the exemplary embodiment, the triplet energy of the second host material T₁(H2) and the triplet energy of the third host material T₁(H3) satisfy a relationship of a numerical formula (Numerical Formula A31) below.

$${T}_{1} ( H 2 ) - {T}_{1} ( H 3 ) > 0.03 eV \qquad ( Numerical Formula A31 )$$

In an exemplary arrangement of the organic EL device according to the exemplary embodiment, HOMO(H2) above satisfies a relationship of a numerical formula (Numerical Formula A10) below.

$$HOMO ( H 2 ) > - 5.7 eV \qquad ( Numerical Formula A10 )$$

In an exemplary arrangement of the organic EL device according to the exemplary embodiment, HOMO(H2) above satisfies a relationship of a numerical formula (Numerical Formula A101) below.

$$HOMO ( H 2 ) \geq - 5.6 eV \qquad ( Numerical Formula A101 )$$

In an exemplary arrangement of the organic EL device according to the exemplary embodiment, HOMO(H2) above satisfies a relationship of a numerical formula (Numerical Formula A102) below.

$$HOMO ( H 2 ) \geq - 5.5 eV \qquad ( Numerical Formula A102 )$$

In an exemplary arrangement of the organic EL device according to the exemplary embodiment, HOMO(H1) above satisfies a relationship of a numerical formula (Numerical Formula A11) below.

$$- 5.6 eV > HOMO ( H 1 ) \qquad ( Numerical Formula A11 )$$

In an exemplary arrangement of the organic EL device according to the exemplary embodiment, HOMO(H1) above satisfies a relationship of a numerical formula (Numerical Formula A111) below.

$$- 5.7 eV > HOMO ( H 1 ) \qquad ( Numerical Formula A111 )$$

In an exemplary arrangement of the organic EL device according to the exemplary embodiment, HOMO(H1) above satisfies the relationship of the numerical formula (Numerical Formula A111) and HOMO(H2) above satisfies the relationship of the numerical formula (Numerical Formula A10).

In an exemplary arrangement of the organic EL device according to the exemplary embodiment, HOMO(H1) above satisfies the relationship of the numerical formula (Numerical Formula A111) and HOMO(H2) above satisfies the relationship of the numerical formula (Numerical Formula A101).

In an exemplary arrangement of the organic EL device according to the exemplary embodiment, HOMO(H1) above satisfies the relationship of the numerical formula (Numerical Formula A111) and HOMO(H2) above satisfies the relationship of the numerical formula (Numerical Formula A102).

### Emitting Region

The organic EL device of the exemplary embodiment includes the emitting region that includes the first emitting layer and the second emitting layer. The emitting region of the exemplary embodiment may consist of the first emitting layer and the second emitting layer, or may further include an organic layer different from the first emitting layer and the second emitting layer.

In the organic EL device according to the exemplary embodiment, the first emitting layer contains the first host material and the second host material, and the second emitting layer contains the third host material. In the organic EL device of the exemplary embodiment, the first material, the first host material, the second host material, and the third host material are mutually different compounds.

In an exemplary arrangement of the organic EL device according to the exemplary embodiment, the first emitting layer contains a first luminescent compound and the second emitting layer contains a second luminescent compound.

In an exemplary arrangement of the organic EL device of the exemplary embodiment, the first luminescent compound and the second luminescent compound are mutually the same or different.

In an exemplary arrangement of the organic EL device according to the exemplary embodiment, the first luminescent compound and the second luminescent compound are each independently a compound that emits light having a maximum peak wavelength of 500 nm or less.

### First Hole Transporting Zone

In an exemplary arrangement of the organic EL device of the exemplary embodiment, a first hole transporting zone, which includes a plurality of organic layers, is disposed between the anode and a first emitting region.

In an exemplary arrangement of the organic EL device of the exemplary embodiment, the first hole transporting zone includes at least the first anode side organic layer.

In an exemplary arrangement of the organic EL device of the exemplary embodiment, the first hole transporting zone includes the first anode side organic layer, a second anode side organic layer, and a third anode side organic layer. In an exemplary arrangement of the organic EL device of the exemplary embodiment, the third anode side organic layer, the second anode side organic layer, and the first anode side organic layer are disposed in this order from a side close to the anode.

### First Anode Side Organic Layer

In the organic EL device of the exemplary embodiment, the first anode side organic layer is disposed between the emitting region and the anode. The first anode side organic layer contains the first material.

The arrangement of the organic EL device of the exemplary embodiment will be further explained in Common Arrangements to Respective Exemplary Embodiments described later.

### Second Exemplary Embodiment

### Organic Electroluminescence Device

An organic electroluminescence device of a second exemplary embodiment includes: an anode; a cathode; and an emitting region disposed between the anode and the cathode, in which: the emitting region includes a first emitting layer; the first emitting layer contains a first host material and a second host material; the first host material and the second host material are mutually different; a triplet energy of the first host material T₁(H1) satisfies a relationship of a numerical formula (Numerical Formula A4) below; and a triplet energy of the second host material T₁(H2) satisfies a relationship of a numerical formula (Numerical Formula A5) below.

$$1.8 eV < {T}_{1} ( H 1 ) < 2.5 eV \qquad ( Numerical Formula A4 )$$

$$1.8 eV < {T}_{1} ( H 2 ) < 2.5 eV \qquad ( Numerical Formula A5 )$$

According to the exemplary embodiment, there can be provided an organic electroluminescence device capable of maintaining luminous efficiency and having a long lifetime. The organic EL device of the exemplary embodiment includes the first emitting layer that contains two host materials satisfying the relationships of Numerical Formula A4 and Numerical Formula A5. Thus, the organic EL device of the exemplary embodiment with improved hole injectability to the first emitting layer maintains luminous efficiency and has a long lifetime compared to an organic EL device including an emitting layer that contains a single host material.

In an exemplary arrangement of the organic EL device according to the exemplary embodiment, the triplet energy of the first host material T₁(H1) satisfies a relationship of a numerical formula (Numerical Formula A41) below, and the triplet energy of the second host material T₁(H2) satisfies a relationship of a numerical formula (Numerical Formula A51) below.

$$2. eV < {T}_{1} ( H 1 ) < 2.3 eV \qquad ( Numerical Formula A41 )$$

$$2. eV < {T}_{1} ( H 2 ) < 2.3 eV \qquad ( Numerical Formula A51 )$$

In an exemplary arrangement of the organic EL device according to the exemplary embodiment, HOMO(H2) above satisfies a relationship of a numerical formula (Numerical Formula A10) below.

$$HOMO ( H 2 ) > - 5.7 eV \qquad ( Numerical Formula A10 )$$

In an exemplary arrangement of the organic EL device according to the exemplary embodiment, HOMO(H2) above satisfies a relationship of a numerical formula (Numerical Formula A101) below.

$$HOMO ( H 2 ) \geq - 5.6 eV \qquad ( Numerical Formula A101 )$$

In an exemplary arrangement of the organic EL device according to the exemplary embodiment, HOMO(H2) above satisfies a relationship of a numerical formula (Numerical Formula A102) below.

$$HOMO ( H 2 ) \geq - 5.5 eV \qquad ( Numerical Formula A102 )$$

In an exemplary arrangement of the organic EL device according to the exemplary embodiment, HOMO(H1) above satisfies a relationship of a numerical formula (Numerical Formula A11) below.

$$- 5.6 eV > HOMO ( H 1 ) \qquad ( Numerical Formula A11 )$$

In an exemplary arrangement of the organic EL device according to the exemplary embodiment, HOMO(H1) above satisfies a relationship of a numerical formula (Numerical Formula A111) below.

$$- 5.7 eV \geq HOMO ( H 1 ) \qquad ( Numerical Formula A111 )$$

In an exemplary arrangement of the organic EL device according to the exemplary embodiment, HOMO(H1) above satisfies the relationship of the numerical formula (Numerical Formula A111) and HOMO(H2) above satisfies the relationship of the numerical formula (Numerical Formula A10).

In an exemplary arrangement of the organic EL device according to the exemplary embodiment, HOMO(H1) above satisfies the relationship of the numerical formula (Numerical Formula A111) and HOMO(H2) above satisfies the relationship of the numerical formula (Numerical Formula A101).

In an exemplary arrangement of the organic EL device according to the exemplary embodiment, HOMO(H1) above satisfies the relationship of the numerical formula (Numerical Formula A111) and HOMO(H2) above satisfies the relationship of the numerical formula (Numerical Formula A102).

### Emitting Region

The organic EL device of the exemplary embodiment includes the emitting region that includes the first emitting layer. The emitting region of the exemplary embodiment may consist of the first emitting layer, or may further include an organic layer different from the first emitting layer.

In the organic EL device according to the exemplary embodiment, the first emitting layer contains the first host material and the second host material. In the organic EL device of the exemplary embodiment, the first host material and the second host material are mutually different compounds.

In an exemplary arrangement of the organic EL device according to the exemplary embodiment, the first emitting layer contains the first luminescent compound.

In an exemplary arrangement of the organic EL device according to the exemplary embodiment, the first luminescent compound is a compound that emits light having a maximum peak wavelength of 500 nm or less.

In an exemplary arrangement of the organic EL device of the exemplary embodiment, the emitting region includes the second emitting layer.

In an exemplary arrangement of the organic EL device of the exemplary embodiment, the second emitting layer contains the third host material, and the first host material, the second host material, and the third host material are mutually different.

In an exemplary arrangement of the organic EL device according to the exemplary embodiment, the triplet energy of the first host material T₁(H1) and the triplet energy of the third host material T₁(H3) satisfy a relationship of a numerical formula (Numerical Formula A2) below, and the triplet energy of the second host material T₁(H2) and the triplet energy of the third host material T₁(H3) satisfy a relationship of a numerical formula (Numerical Formula A3) below.

$${T}_{1} ( H 1 ) > {T}_{1} ( H 3 ) \qquad ( Numerical Formula A2 )$$

$${T}_{1} ( H 2 ) > {T}_{1} ( H 3 ) \qquad ( Numerical Formula A3 )$$

The organic EL device according to the exemplary embodiment, in which the first emitting layer and the second emitting layer satisfying the relationships of Numerical Formula A2 and Numerical Formula A3 are layered, is expected to have the effect obtained by layering the emitting layers as in the first exemplary embodiment.

In an exemplary arrangement of the organic EL device according to the exemplary embodiment, the first emitting layer is disposed between the anode and the second emitting layer.

In an exemplary arrangement of the organic EL device according to the exemplary embodiment, the second emitting layer contains the second luminescent compound. In an exemplary arrangement of the organic EL device of the exemplary embodiment, the first luminescent compound and the second luminescent compound are mutually the same or different.

In an exemplary arrangement of the organic EL device according to the exemplary embodiment, the second luminescent compound is a compound that emits light having a maximum peak wavelength of 500 nm or less.

### First Hole Transporting Zone

In an exemplary arrangement of the organic EL device of the exemplary embodiment, a first hole transporting zone, which includes a plurality of organic layers, is disposed between the anode and a first emitting region.

In an exemplary arrangement of the organic EL device of the exemplary embodiment, the first hole transporting zone includes a first anode side organic layer.

In an exemplary arrangement of the organic EL device of the exemplary embodiment, the first hole transporting zone includes the first anode side organic layer, a second anode side organic layer, and a third anode side organic layer. In an exemplary arrangement of the organic EL device of the exemplary embodiment, the third anode side organic layer, the second anode side organic layer, and the first anode side organic layer are disposed in this order from a side close to the anode.

### First Anode Side Organic Layer

In an exemplary arrangement of the organic EL device of the exemplary embodiment, the first anode side organic layer is disposed between the emitting region and the anode.

In an exemplary arrangement of the organic EL device of the exemplary embodiment, the first emitting layer is in direct contact with the first anode side organic layer.

In an exemplary arrangement of the organic EL device of the exemplary embodiment, the first anode side organic layer contains a first material.

In an exemplary arrangement of the organic EL device of the exemplary embodiment, the first material, the first host material, and the second host material are mutually different. In an exemplary arrangement of the organic EL device of the exemplary embodiment, when the emitting region includes the second emitting layer, the first material, the first host material, the second host material, and the third host material are mutually different.

In an exemplary arrangement of the organic EL device of the exemplary embodiment, an energy level of a highest occupied molecular orbital of the first material HOMO(HT1), an energy level of a highest occupied molecular orbital of the first host material HOMO(H1), and an energy level of a highest occupied molecular orbital of the second host material HOMO(H2) satisfy a relationship of a numerical formula (Numerical Formula A1) below.

$$HOMO ( HT 1 ) > HOMO ( H 2 ) > HOMO ( H 1 ) \qquad ( Numerical Formula A1 )$$

The arrangement of the organic EL device of the exemplary embodiment will be further explained in Common Arrangements to Respective Exemplary Embodiments described later.

### Third Exemplary Embodiment

### Composition

A composition according to a third exemplary embodiment contains a first compound and a second compound, in which the first compound and the second compound are mutually different compounds, and the first compound and the second compound each independently include, in a molecule, at least one of a structure of Condition (i) or a structure of Condition (ii) below.

Condition (i): a biphenyl structure including a first benzene ring and a second benzene ring linked to each other with a single bond, the first benzene ring and the second benzene ring in the biphenyl structure being further linked to each other by cross-linking at at least one site other than the single bond.

Condition (ii): a first linking structure including a benzene ring and a naphthalene ring linked to each other with a single bond, the benzene ring and the naphthalene ring in the first linking structure being each independently further fused or not fused with a monocyclic ring or fused ring, the benzene ring and the naphthalene ring in the first linking structure being further linked to each other by cross-linking at at least one site other than the single bond.

The composition of the exemplary embodiment is usable for the organic EL device. The organic EL device in which the composition of the exemplary embodiment is used has improved device performance.

The emitting layer of the organic EL device can be formed by using the composition of the exemplary embodiment. Using the composition of the exemplary embodiment in the emitting layer of the organic EL device allows the organic EL device to maintain luminous efficiency and have a long lifetime.

In an exemplary arrangement of the composition of the exemplary embodiment, at least one of the first compound or the second compound includes, in a molecule, the structure of Condition (i) above.

In an exemplary arrangement of the composition of the exemplary embodiment, the first benzene ring and the second benzene ring in the biphenyl structure of Condition (i) above are further linked to each other by the cross-linking of Condition (i) above at one site other than the single bond.

When the composition of the exemplary embodiment, in which at least one of the first compound or the second compound includes the biphenyl structure including such cross-linking, is used in the emitting layer of the organic EL device, deterioration in chromaticity of the organic EL device is expected to be inhibited.

In an exemplary arrangement of the composition of the exemplary embodiment, the first benzene ring and the second benzene ring in the biphenyl structure of Condition (i) above are further linked to each other by the cross-linking of Condition (i) above at two sites other than the single bond.

In an exemplary arrangement of the composition of the exemplary embodiment, the cross-linking of Condition (i) above includes a double bond.

In an exemplary arrangement of the composition of the exemplary embodiment, the cross-linking of Condition (i) above includes no double bond.

In an exemplary arrangement of the composition of the exemplary embodiment, at least one of the first compound or the second compound includes, in a molecule, the structure of Condition (i) above, in which the first benzene ring and the second benzene ring in the biphenyl structure are further linked to each other by the cross-linking at two sites other than the single bond and the cross-linking includes no double bond. When the composition of the exemplary embodiment, in which at least one of the first compound or the second compound includes the biphenyl structure including such cross-linking, is used in the emitting layer of the organic EL device, deterioration in chromaticity of the organic EL device is expected to be inhibited.

For instance, assuming that the first benzene ring and the second benzene ring in the biphenyl structure represented by a formula (BP1) below are further linked to each other by cross-linking at at least one site other than the single bond, the biphenyl structure is exemplified by linking structures (fused rings) represented by formulae (BP11) to (BP15) below.

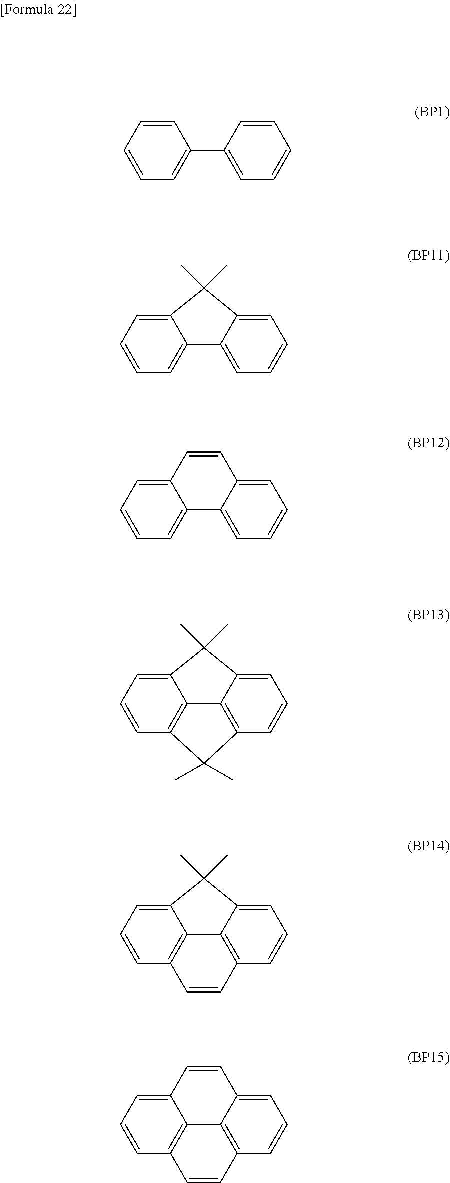

The formula (BP11) represents a linking structure in which the first benzene ring and the second benzene ring are linked to each other at one site other than the single bond by cross-linking including no double bond.

The formula (BP12) represents a linking structure in which the first benzene ring and the second benzene ring are linked to each other at one site other than the single bond by cross-linking including a double bond.

The formula (BP13) represents a linking structure in which the first benzene ring and the second benzene ring are linked to each other at two sites other than the single bond by cross-linking including no double bond.

The formula (BP14) represents a linking structure in which the first benzene ring and the second benzene ring are linked to each other by cross-linking including no double bond at one of two sites other than the single bond, and the first benzene ring and the second benzene ring are linked to each other by cross-linking including a double bond at the other of the two sites other than the single bond.

The formula (BP15) represents a linking structure in which the first benzene ring and the second benzene ring are linked to each other at two sites other than the single bond by cross-linking including a double bond.

In an exemplary arrangement of the composition of the exemplary embodiment, at least one of the first compound or the second compound includes, in a molecule, the structure of Condition (ii) above.

When the composition of the exemplary embodiment, in which at least one of the first compound or the second compound includes the linking structure including such cross-linking, is used in the emitting layer of the organic EL device, deterioration in chromaticity of the organic EL device is expected to be inhibited.

At least one of the first compound or the second compound in the above case is only required to have a first linking structure as the minimum unit in a molecule, the first linking structure including a benzene ring and a naphthalene ring linked to each other with a single bond (occasionally referred to as a benzene-naphthalene linking structure), the first linking structure being as represented by a formula (X1) or a formula (X2) below. The benzene ring may be fused with a further monocyclic ring or fused ring, and the naphthalene ring may be fused with a further monocyclic ring or fused ring. For instance, also in a case where at least one of the first compound or the second compound has, in a molecule, a second linking structure including a naphthalene ring and a naphthalene ring linked to each other with a single bond (occasionally referred to as a naphthalene-naphthalene linking structure) and being as represented by a formula (X3), a formula (X4), or a formula (X5) below, the naphthalene-naphthalene linking structure is regarded as including the benzene-naphthalene linking structure since one of the naphthalene rings includes a benzene ring.

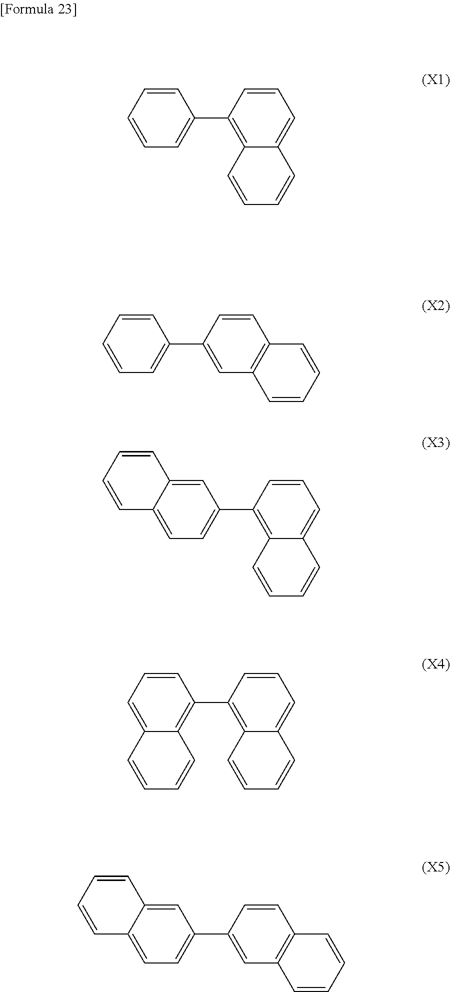

In an exemplary arrangement of the composition of the exemplary embodiment, the cross-linking of Condition (ii) above includes a double bond. Specifically, the composition of the exemplary embodiment also preferably has a structure in which the benzene ring and the naphthalene ring are further linked to each other at any other site than the single bond by the cross-linking structure including a double bond.

Assuming that the benzene ring and the naphthalene ring in the first linking structure (benzene-naphthalene linking structure) are further linked to each other at at least one site other than the single bond by cross-linking, for instance, a linking structure (fused ring) represented by a formula (X11) below is obtained in a case of the formula (X1), and a linking structure (fused ring) represented by a formula (X31) below is obtained in a case of the formula (X3).

Assuming that the benzene ring and the naphthalene ring in the benzene-naphthalene linking structure are further linked to each other at any other site than the single bond by cross-linking including a double bond, for instance, a linking structure (fused ring) represented by a formula (X12) below is obtained in a case of the formula (X1), a linking structure (fused ring) represented by a formula (X21), formula (X22), or formula (X23) below is obtained in a case of the formula (X2), a linking structure (fused ring) represented by a formula (X41) below is obtained in a case of the formula (X4), and a linking structure (fused ring) represented by a formula (X51) below is obtained in a case of the formula (X5).

Assuming that the benzene ring and the naphthalene ring in the benzene-naphthalene linking structure are further linked to each other at at least one site other than the single bond by cross-linking including a hetero atom (e.g., an oxygen atom), for instance, a linking structure (fused ring) represented by a formula (X13) below is obtained in a case of the formula (X1).

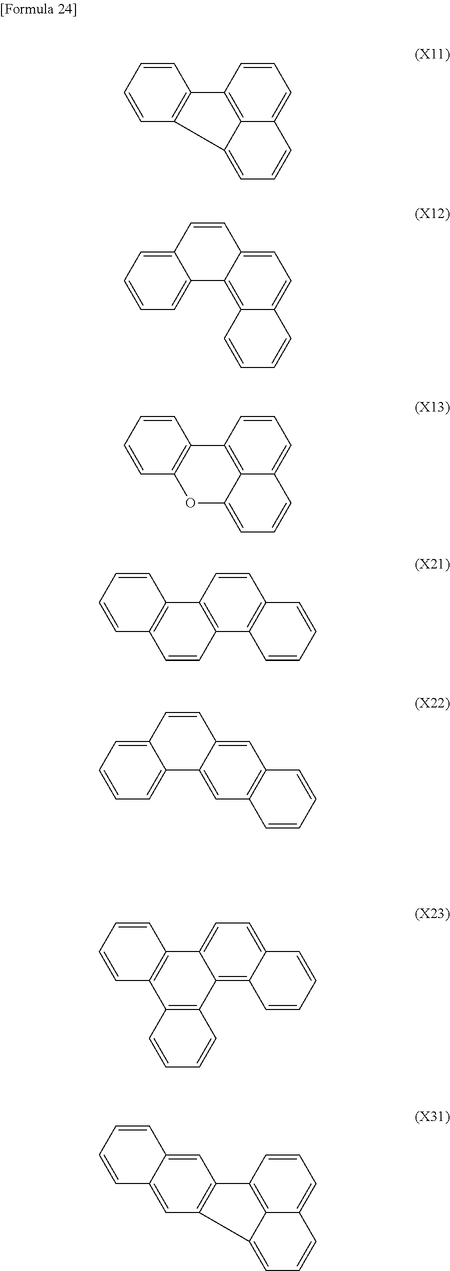

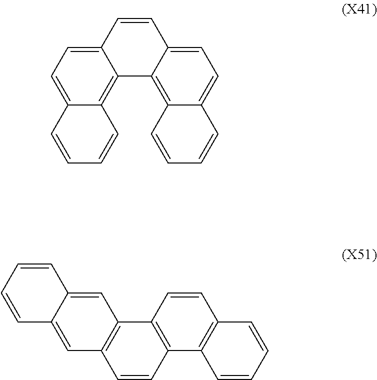

In an exemplary arrangement of the composition of the exemplary embodiment, the first compound and the second compound are each independently a compound selected from the group consisting of a compound represented by a formula (H11), a compound represented by a formula (H12), a compound represented by a formula (H13), a compound represented by a formula (H14), a compound represented by a formula (H15), and a compound represented by a formula (H16) below.

### Compound Represented by Formula (H11)

The compound represented by the formula (H11) will be described below.

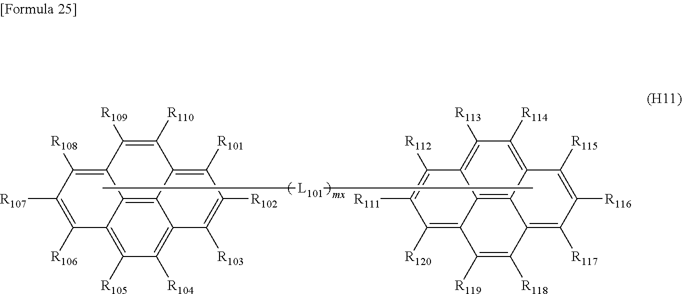

In the formula (H11):
R₁₀₁ to R₁₁₀ and R₁₁₁ to R₁₂₀ are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a substituted or unsubstituted aralkyl group having 7 to 50 carbon atoms, a group represented by —C(═O) R₈₀₁, a group represented by —COOR₈₀₂, a halogen atom, a cyano group, a nitro group, a substituted or unsubstituted aryl group having 6 to 50 ring carbon atoms, or a substituted or unsubstituted heterocyclic group having 5 to 50 ring atoms;
one of R₁₀₁ to R₁₁₀ represents a bonding position to L₁₀₁, and one of R₁₁₁ to R₁₂₀ represents a bonding position to L₁₀₁;
L₁₀₁ is a single bond, a substituted or unsubstituted arylene group having 6 to 24 ring carbon atoms, or a substituted or unsubstituted divalent heterocyclic group having 5 to 24 ring atoms;
mx is 0, 1, 2, 3, 4, or 5; and
when two or more L₁₀₁ are present, the two or more L₁₀₁ are mutually the same or different.

In the first compound and the second compound, R₉₀₁, R₉₀₂, R₉₀₃, R₉₀₄, R₉₀₅, R₉₀₆, R₉₀₇, R₈₀₁ and R₈₀₂ are each independently a hydrogen atom, a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms;
when a plurality of R₉₀₁ are present, the plurality of R₉₀₁ are mutually the same or different;
when a plurality of R₉₀₂ are present, the plurality of R₉₀₂ are mutually the same or different;
when a plurality of R₉₀₃ are present, the plurality of R₉₀₃ are mutually the same or different;
when a plurality of R₉₀₄ are present, the plurality of R₉₀₄ are mutually the same or different;
when a plurality of R₉₀₅ are present, the plurality of R₉₀₅ are mutually the same or different;
when a plurality of R₉₀₆ are present, the plurality of R₉₀₆ are mutually the same or different;
when a plurality of R₉₀₇ are present, the plurality of R₉₀₇ are mutually the same or different;
when a plurality of R₈₀₁ are present, the plurality of R₈₀₁ are mutually the same or different; and
when a plurality of R₈₀₂ are present, the plurality of R₈₀₂ are mutually the same or different.

In an exemplary arrangement of the composition of the exemplary embodiment, the compound represented by the formula (H11) is a compound represented by a formula (H111) below.

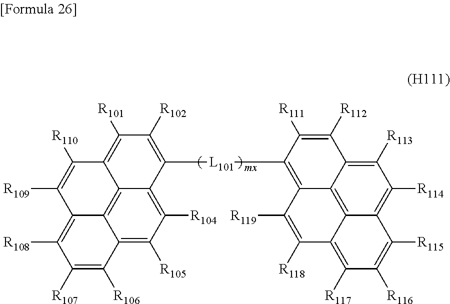

In the formula (H111):
R₁₀₁, R₁₀₂, R₁₀₄ to R₁₁₀, and R₁₁₁ to R₁₁₉ are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by—S—(R₉₀₅), a substituted or unsubstituted aralkyl group having 7 to 50 carbon atoms, a group represented by —C(═O) R₈₀₁, a group represented by —COOR₈₀₂, a halogen atom, a cyano group, a nitro group, a substituted or unsubstituted aryl group having 6 to 50 ring carbon atoms, or a substituted or unsubstituted heterocyclic group having 5 to 50 ring atoms; and
L₁₀₁ and mx respectively represent the same as L₁₀₁ and mx in the formula (H11).

In an exemplary arrangement of the composition of the exemplary embodiment, mx is 1 or 2.

In an exemplary arrangement of the composition of the exemplary embodiment, L₁₀₁ is a substituted or unsubstituted arylene group having 6 to 24 ring carbon atoms.

In an exemplary arrangement of the composition of the exemplary embodiment, at least one of the first compound or the second compound is a compound including, in a molecule, only two pyrene rings (occasionally referred to as a bispyrene compound).

In an exemplary arrangement of the composition of the exemplary embodiment, the compound represented by the formula (H11) is the bispyrene compound.

### Compound Represented by Formula (H12)

The compound represented by the formula (H12) will be described below.

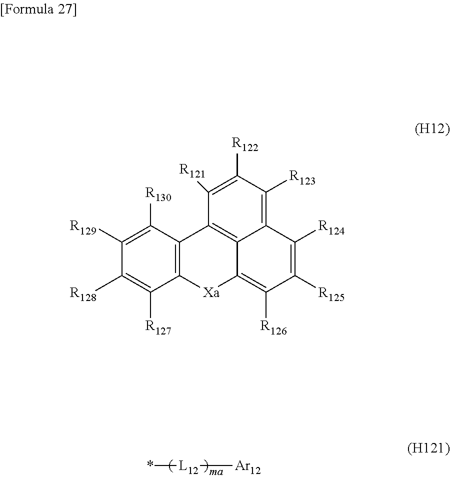

In the formula (H12):
Xa is an oxygen atom, a sulfur atom, C(R₁₂₀₁)(R₁₂₀₂), or Si(R₁₂₀₃)(R₁₂₀₄);
R₁₂₀₁ to R₁₂₀₄ are each independently a hydrogen atom, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a halogen atom, a cyano group, a nitro group, a substituted or unsubstituted aryl group having 6 to 50 ring carbon atoms, or a substituted or unsubstituted heterocyclic group having 5 to 50 ring atoms;
at least one combination of adjacent two or more of R₁₂₁ to R₁₃₀ are mutually bonded to form a substituted or unsubstituted monocyclic ring, mutually bonded to form a substituted or unsubstituted fused ring, or not mutually bonded;
R₁₂₁ to R₁₃₀ forming neither the substituted or unsubstituted monocyclic ring nor the substituted or unsubstituted fused ring are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by—S—(R₉₀₅), a group represented by —N(R₉₀₆)(R₉₀₇), a substituted or unsubstituted aralkyl group having 7 to 50 carbon atoms, a group represented by —C(═O) R₈₀₁, a group represented by —COOR₈₀₂, a halogen atom, a nitro group, a substituted or unsubstituted aryl group having 6 to 50 ring carbon atoms, a substituted or unsubstituted heterocyclic group having 5 to 50 ring atoms, or a group represented by the formula (H121);
at least one of R₁₂₁ to R₁₃₀ is a group represented by a formula (H121) above;
when a plurality of groups represented by the formula (H121) are present, the plurality of groups represented by the formula (H121) are mutually the same or different;
L₁₂ is a single bond, a substituted or unsubstituted arylene group having 6 to 50 ring carbon atoms, or a substituted or unsubstituted divalent heterocyclic group having 5 to 50 ring atoms;
ma is 0, 1, 2 or 3;
when two or more L₁₂ are present, the two or more L₁₂ are mutually the same or different;
Ar₁₂ is a substituted or unsubstituted aryl group having 6 to 50 ring carbon atoms, or a substituted or unsubstituted heterocyclic group having 5 to 50 ring atoms;
when two or more Ar₁₂ are present, the two or more Ar₁₂ are mutually the same or different; and
* in the formula (H121) represents a bonding position.

In an exemplary arrangement of the composition of the exemplary embodiment, L₁₂ is a single bond, a substituted or unsubstituted arylene group having 6 to 15 ring carbon atoms, or a substituted or unsubstituted divalent heterocyclic group having 5 to 15 ring atoms.

In an exemplary arrangement of the composition of the exemplary embodiment, Ar₁₂ is a substituted or unsubstituted aryl group having four or more rings, or a substituted or unsubstituted heterocyclic group having four or more rings.

In an exemplary arrangement of the composition of the exemplary embodiment, Ar₁₂ is a substituted or unsubstituted aryl group having 4 or more and 6 or less rings, or a substituted or unsubstituted heterocyclic group having 4 or more and 6 or less rings.

In an exemplary arrangement of the composition of the exemplary embodiment, Ar₁₂ is a substituted or unsubstituted aryl group having four or more rings.

In an exemplary arrangement of the composition of the exemplary embodiment, Ar₁₂ is a substituted or unsubstituted aryl group having 4 or more and 6 or less rings.

In an exemplary arrangement of the composition of the exemplary embodiment, R₁₂₉ is a group represented by the formula (H121).

In an exemplary arrangement of the composition of the exemplary embodiment, Xa is an oxygen atom.

In an exemplary arrangement of the composition of the exemplary embodiment, the compound represented by the formula (H12) is a compound represented by a formula (H122) below.

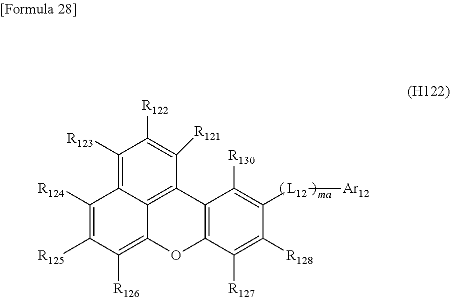

In the formula (H122):
R₁₂₁ to R₁₂₈ and R₁₃₀ are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by—S—(R₉₀₅), a group represented by —N(R₉₀₆)(R₉₀₇), a substituted or unsubstituted aralkyl group having 7 to 50 carbon atoms, a group represented by —C(═O) R₈₀₁, a group represented by —COOR₈₀₂, a halogen atom, a nitro group, a substituted or unsubstituted aryl group having 6 to 50 ring carbon atoms, or a substituted or unsubstituted heterocyclic group having 5 to 50 ring atoms; and
Ar₁₂, L₁₂, and ma respectively represent the same as Ar₁₂, L₁₂, and ma in the formula (H121).

In an exemplary arrangement of the composition of the exemplary embodiment, the compound represented by the formula (H12) is a compound represented by a formula (H123) below.

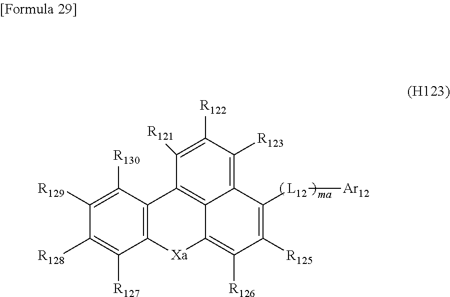

In the formula (H123):
Xa is an oxygen atom, a sulfur atom, C(R₁₂₀₁)(R₁₂₀₂), or Si(R₁₂₀₃)(R₁₂₀₄); R₁₂₀₁ to R₁₂₀₄ each independently represent the same as R₁₂₀₁ to R₁₂₀₄ in the formula (H12);
R₁₂₁ to R₁₂₃ and R₁₂₅ to R₁₃₀ are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by—S—(R₉₀₅), a group represented by —N(R₉₀₆)(R₉₀₇), a substituted or unsubstituted aralkyl group having 7 to 50 carbon atoms, a group represented by —C(═O) R₈₀₁, a group represented by —COOR₈₀₂, a halogen atom, a nitro group, a substituted or unsubstituted aryl group having 6 to 50 ring carbon atoms, or a substituted or unsubstituted heterocyclic group having 5 to 50 ring atoms; and
Ar₁₂, L₁₂, and ma respectively represent the same as Ar₁₂, L₁₂, and ma in the formula (H121).

In an exemplary arrangement of the composition of the exemplary embodiment, ma is 1 or 2.

### Compound Represented by Formula (H13)

The compound represented by the formula (H13) will be described below.

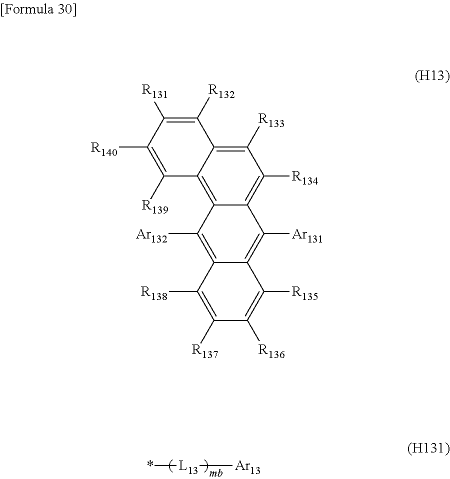

In the formula (H13):
R₁₃₁ to R₁₄₀, Ar₁₃₁, and Ar₁₃₂ are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by—S—(R₉₀₅), a substituted or unsubstituted aralkyl group having 7 to 50 carbon atoms, a group represented by —C(═O) R₈₀₁, a group represented by —COOR₈₀₂, a halogen atom, a cyano group, a nitro group, a substituted or unsubstituted aryl group having 6 to 50 ring carbon atoms, a substituted or unsubstituted heterocyclic group having 5 to 50 ring atoms, or a group represented by a formula (H131) above;
at least one of R₁₃₁ to R₁₄₀, Ar₁₃₁, or Ar₁₃₂ is a group represented by the formula (H131);
when a plurality of groups represented by the formula (H131) are present, the plurality of groups represented by the formula (H131) are mutually the same or different;
L₁₃ is a single bond, a substituted or unsubstituted arylene group having 6 to 50 ring carbon atoms, or a substituted or unsubstituted divalent heterocyclic group having 5 to 50 ring atoms;
Ar₁₃ is a substituted or unsubstituted aryl group having 6 to 50 ring carbon atoms, or a substituted or unsubstituted heterocyclic group having 5 to 50 ring atoms;
mb is 0, 1, 2, 3, 4, or 5;
when two or more L₁₃ are present, the two or more L₁₃ are mutually the same or different;
when two or more Ar₁₃ are present, the two or more Ar₁₃ are mutually the same or different; and
* in the formula (H131) represents a bonding position to a benz[a]anthracene ring in the formula (H13).

In an exemplary arrangement of the composition of the exemplary embodiment, at least one of Ar₁₃₁ or Ar₁₃₂ is a group represented by the formula (H131).

In an exemplary arrangement of the composition of the exemplary embodiment, the compound represented by the formula (H13) is a compound represented by a formula (H132) or (H133) below.

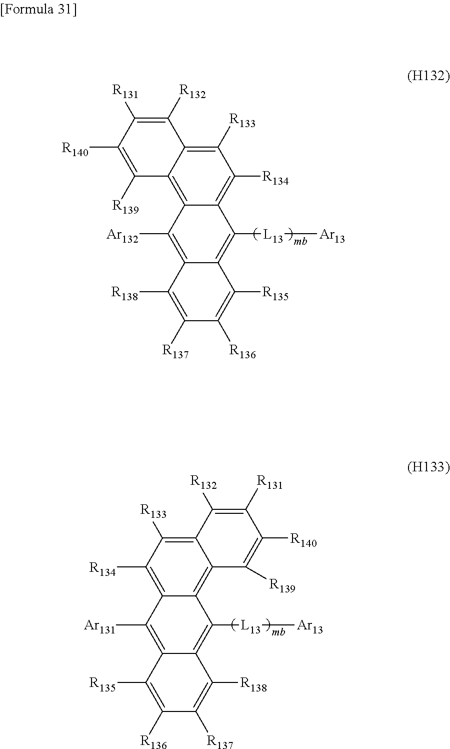

In the formulae (H132) and (H133):
R₁₃₁ to R₁₄₀, Ar₁₃₁, and Ar₁₃₂ are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by—S—(R₉₀₅), a substituted or unsubstituted aralkyl group having 7 to 50 carbon atoms, a group represented by —C(═O) R₈₀₁, a group represented by —COOR₈₀₂, a halogen atom, a cyano group, a nitro group, a substituted or unsubstituted aryl group having 6 to 50 ring carbon atoms, or a substituted or unsubstituted heterocyclic group having 5 to 50 ring atoms; and
L₁₃, Ar₁₃, and mb respectively represent the same as L₁₃, Ar₁₃, and mb in the formula (H131).

In an exemplary arrangement of the composition of the exemplary embodiment, mb is 0, 1, or 2.

### Compound Represented by Formula (H14)

The compound represented by the formula (H14) will be described below.

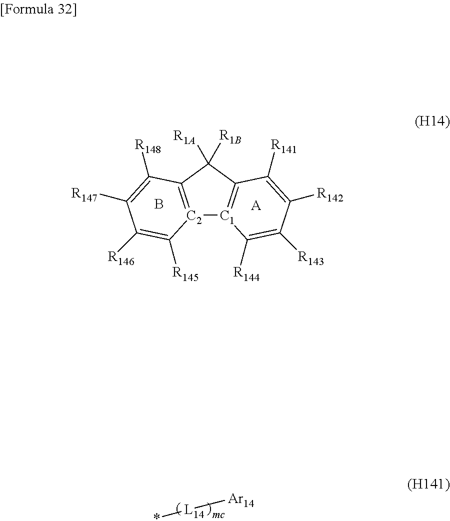

In the formula (H14):
R_{1A} and R_{1B} are each independently a substituted or unsubstituted alkyl group having 1 to 15 carbon atoms, a substituted or unsubstituted aryl group having 6 to 17 ring carbon atoms, or a substituted or unsubstituted heterocyclic group having 5 to 17 ring atoms;
at least one of R_{1A} or R_{1B} is a substituted or unsubstituted alkyl group having 1 to 15 carbon atoms;
at least one combination of a combination of adjacent two or more of R₁₄₁ to R₁₄₄ and a combination of adjacent two or more of R₁₄₅ to R₁₄₈ are mutually bonded to form a substituted or unsubstituted monocyclic ring, or mutually bonded to form a substituted or unsubstituted fused ring;
when a substituted or unsubstituted monocyclic ring or a substituted or unsubstituted fused ring is formed with the ring A, a group represented by a formula (H141) above is bonded to a carbon atom bonded to R₁₄₂ or, of carbon atoms forming the monocyclic ring with the ring A and the fused ring with the ring A, a carbon atom farthest from a carbon atom C₁ of the ring A, the carbon atom C₁ being bonded with a single bond to a carbon atom C₂ of a ring B;
when a substituted or unsubstituted monocyclic ring or a substituted or unsubstituted fused ring is formed with not the ring A but the ring B, a group represented by the formula (H141) is bonded to a carbon atom bonded to R₁₄₂; and
R₁₄₂ not being the group represented by the formula (H141), and R₁₄₁, R₁₄₃, R₁₄₄, and R₁₄₅ to R₁₄₈ forming neither the substituted or unsubstituted monocyclic ring nor the substituted or unsubstituted fused ring are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a substituted or unsubstituted aralkyl group having 7 to 50 carbon atoms, a group represented by —C(═O) R₈₀₁, a group represented by —COOR₈₀₂, a halogen atom, a cyano group, a nitro group, a substituted or unsubstituted aryl group having 6 to 17 ring carbon atoms, or a substituted or unsubstituted heterocyclic group having 5 to 17 ring atoms; and in the formula (H141):
Ar₁₄ is a substituted or unsubstituted aryl group having four or more fused rings or a substituted or unsubstituted heterocyclic group having four or more fused rings;
L₁₄ is a single bond, a substituted or unsubstituted arylene group having 6 to 17 ring carbon atoms, or a substituted or unsubstituted divalent heterocyclic group having 5 to 17 ring atoms;
mc is 0, 1, or 2;
* represents a bonding position to an atom forming a ring of the formula (H14); and
the compound represented by the formula (H14) does not have, in a molecule, three or more groups of a substituted or unsubstituted aryl group having four or more fused rings and a substituted or unsubstituted heterocyclic group having four or more fused rings.

In an exemplary arrangement of the composition of the exemplary embodiment, R₁₄₂ is a group represented by the formula (H141).

In an exemplary arrangement of the composition of the exemplary embodiment, R₉₀₁, R₉₀₂, R₉₀₃, R₉₀₄, R₉₀₅, R₉₀₆, R₉₀₇, R₈₀₁ and R₈₀₂ in the compound represented by the formula (H14) are each independently a hydrogen atom, a substituted or unsubstituted alkyl group having 1 to 50 carbon atoms, a substituted or unsubstituted cycloalkyl group having 3 to 50 ring carbon atoms, a substituted or unsubstituted aryl group having 6 to 17 ring carbon atoms, or a substituted or unsubstituted heterocyclic group having 5 to 17 ring atoms.

In an exemplary arrangement of the composition of the exemplary embodiment, the compound represented by the formula (H14) is a compound represented by a formula (H142), (H143), or (H144) below.

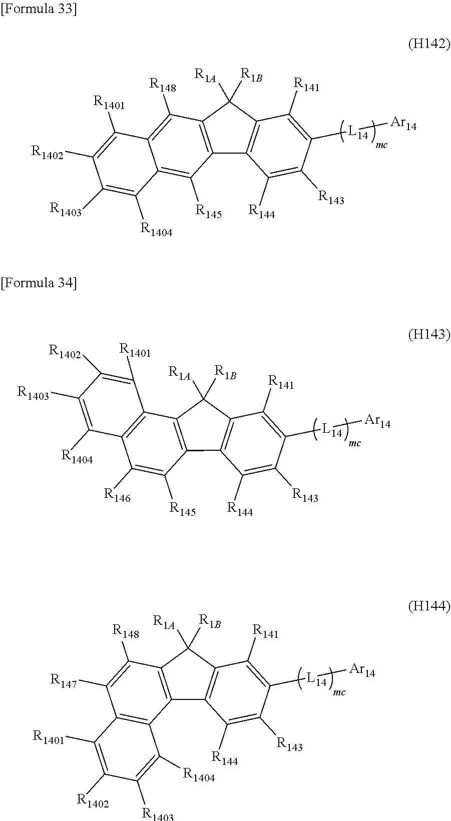

In the formula (H142), (H143), or (H144):
R_{1A}, R_{1B}, R₁₄₁, R₁₄₃, R₁₄₄, R₁₄₅, R₁₄₆, R₁₄₇ and R₁₄₈ respectively represent the same as R_{1A}, R_{1B}, R₁₄₁, R₁₄₃, R₁₄₄, R₁₄₅, R₁₄₆, R₁₄₇ and R₁₄₈ in the formula (H14);
Ar₁₄, L₁₄, and mc respectively represent the same as Ar₁₄, L₁₄, and mc in the formula (H141);
none of one or more combinations of adjacent two or more of R₁₄₀₁ to R₁₄₀₄ are bonded to each other; and
R₁₄₀₁ to R₁₄₀₄ are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a substituted or unsubstituted aralkyl group having 7 to 50 carbon atoms, a group represented by —C(═O) R₈₀₁, a group represented by —COOR₈₀₂, a halogen atom, a cyano group, a nitro group, a substituted or unsubstituted aryl group having 6 to 17 ring carbon atoms, or a substituted or unsubstituted heterocyclic group having 5 to 17 ring atoms.

In an exemplary arrangement of the composition of the exemplary embodiment, mc is 0, 1, or 2.

### Compound Represented by Formula (H15)

The compound represented by the formula (H15) will be described below.

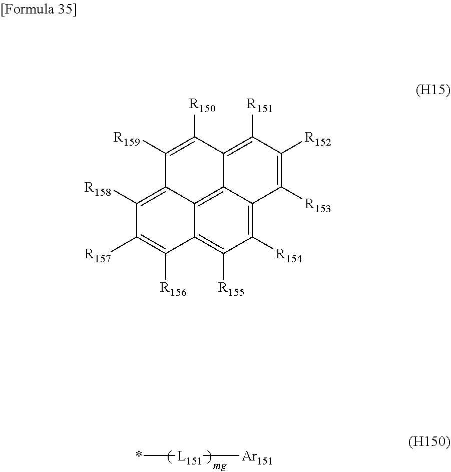

In the formula (H15):
R₁₅₀ to R₁₅₉ are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a substituted or unsubstituted aralkyl group having 7 to 50 carbon atoms, a group represented by—C(═O) R₈₀₁, a group represented by —COOR₈₀₂, a halogen atom, a cyano group, a nitro group, a substituted or unsubstituted aryl group having 6 to 50 ring carbon atoms, a substituted or unsubstituted heterocyclic group having 5 to 50 ring atoms, or a group represented by a formula (H150) above;
at least one of R₁₅₀ to R₁₅₉ is a group represented by the formula (H150);
when a plurality of groups represented by the formula (H150) are present, the plurality of groups represented by the formula (H150) are mutually the same or different;
L₁₅₁ is a single bond, a substituted or unsubstituted arylene group having 6 to 50 ring carbon atoms, or a substituted or unsubstituted divalent heterocyclic group having 5 to 50 ring atoms;
Ar₁₅₁ is a substituted or unsubstituted aryl group having 6 to 50 ring carbon atoms, or a substituted or unsubstituted heterocyclic group having 5 to 50 ring atoms;
mg is 0, 1, 2, 3, 4, or 5;
when two or more L₁₅₁ are present, the two or more L₁₅₁ are mutually the same or different;
when two or more Ar₁₅₁ are present, the two or more Ar₁₅₁ are mutually the same or different; and
* in the formula (H150) represents a bonding position to a pyrene ring in the formula (H15).

In an exemplary arrangement of the composition of the exemplary embodiment, R₁₅₃ in the compound represented by the formula (H15) is a group represented by the formula (H150).

In an exemplary arrangement of the composition of the exemplary embodiment, L₁₅₁ is a single bond or a substituted or unsubstituted arylene group having 6 to 50 ring carbon atoms, and Ar₁₅₁ is a substituted or unsubstituted aryl group having 6 to 50 ring carbon atoms.

In an exemplary arrangement of the composition of the exemplary embodiment, L₁₅₁ is a single bond or a substituted or unsubstituted arylene group having 6 to 14 ring carbon atoms, and Ar₁₅₁ is a substituted or unsubstituted aryl group having 6 to 14 ring carbon atoms.

In an exemplary arrangement of the composition of the exemplary embodiment, the group represented by the formula (H150) is a group represented by a formula (H151) below.

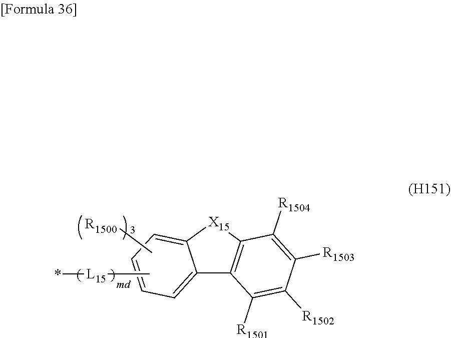

In the formula (H151):
X₁₅ is an oxygen atom or a sulfur atom;
L₁₅ is a single bond, a substituted or unsubstituted arylene group having 6 to 50 ring carbon atoms, or a substituted or unsubstituted divalent heterocyclic group having 5 to 50 ring atoms;
md is 0, 1, 2, 3, 4, or 5;
when two or more L₁₅ are present, the two or more L₁₅ are mutually the same or different;
at least one combination of adjacent two or more of R₁₅₀₀ to R₁₅₀₄ are mutually bonded to form a substituted or unsubstituted monocyclic ring, mutually bonded to form a substituted or unsubstituted fused ring, or not mutually bonded;
R₁₅₀₀ to R₁₅₀₄ forming neither the substituted or unsubstituted monocyclic ring nor the substituted or unsubstituted fused ring are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by—S—(R₉₀₅), a substituted or unsubstituted aralkyl group having 7 to 50 carbon atoms, a group represented by —C(═O) R₈₀₁, a group represented by —COOR₈₀₂, a halogen atom, a cyano group, a nitro group, a substituted or unsubstituted aryl group having 6 to 50 ring carbon atoms, or a substituted or unsubstituted heterocyclic group having 5 to 50 ring atoms;
a plurality of R₁₅₀₀ are mutually the same or different;
when a plurality of groups represented by the formula (H151) are present, the plurality of groups represented by the formula (H151) are mutually the same or different; and
* in the formula (H151) represents a bonding position to the pyrene ring in the formula (H15).

In an exemplary arrangement of the composition of the exemplary embodiment, the compound represented by the formula (H15) is a compound represented by a formula (H152) below. When R₁₅₃ is a group represented by the formula (H151), the compound represented by the formula (H15) is represented by a formula (H152) below.

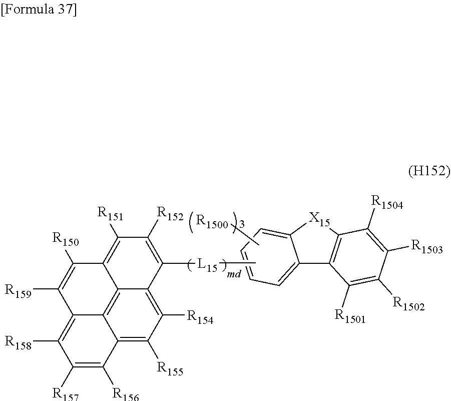

In the formula (H152):
R₁₅₀ to R₁₅₂ and R₁₅₄ to R₁₅₉ are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a substituted or unsubstituted aralkyl group having 7 to 50 carbon atoms, a group represented by —C(═O) R₈₀₁, a group represented by —COOR₈₀₂, a halogen atom, a cyano group, a nitro group, a substituted or unsubstituted aryl group having 6 to 50 ring carbon atoms, or a substituted or unsubstituted heterocyclic group having 5 to 50 ring atoms;
X₁₅, L₁₅, and md respectively represent the same as X₁₅, L₁₅, and md in the formula (H151); and
R₁₅₀₀ to R₁₅₀₄ each independently represent the same as R₁₅₀₀ to R₁₅₀₄ in the formula (H151).

In an exemplary arrangement of the composition of the exemplary embodiment, md is 0, 1, or 2.

In an exemplary arrangement of the composition of the exemplary embodiment, when md is 0, the compound represented by the formula (H152) is represented by a formula (H153) below.

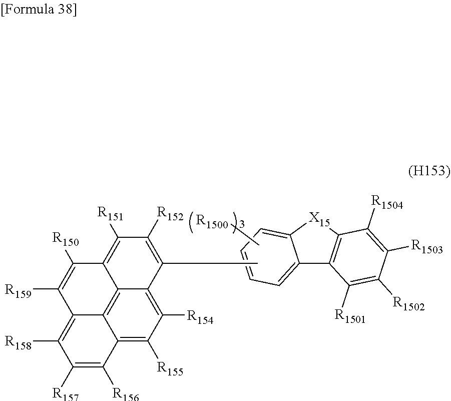

In the formula (H153), R₁₅₀ to R₁₅₂, R₁₅₄ to R₁₅₉, R₁₅₀₀ to R₁₅₀₄, and X₁₅ respectively represent the same as R₁₅₀ to R₁₅₂, R₁₅₄ to R₁₅₉, R₁₅₀₀ to R₁₅₀₄, and X₁₅ in the formula (H152).

In an exemplary arrangement of the composition of the exemplary embodiment, at least one of the first compound or the second compound is a compound including, in a molecule, only one pyrene ring (occasionally referred to as a monopyrene compound).

In an exemplary arrangement of the composition of the exemplary embodiment, the compound represented by the formula (H15) is the monopyrene compound.

### Compound Represented by Formula (H16)

The compound represented by the formula (H16) will be described below.

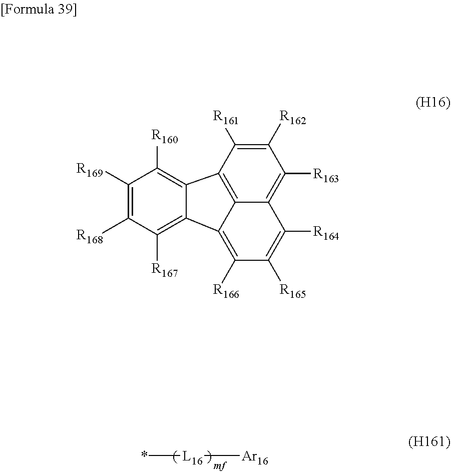

In the formula (H16):
at least one combination of adjacent two or more of R₁₆₀ to R₁₆₉ are mutually bonded to form a substituted or unsubstituted monocyclic ring, mutually bonded to form a substituted or unsubstituted fused ring, or not mutually bonded;
R₁₆₀ to R₁₆₉ forming neither the substituted or unsubstituted monocyclic ring nor the substituted or unsubstituted fused ring are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by—S—(R₉₀₅), a substituted or unsubstituted aralkyl group having 7 to 50 carbon atoms, a group represented by —C(═O) R₈₀₁, a group represented by —COOR₈₀₂, a halogen atom, a cyano group, a nitro group, a substituted or unsubstituted aryl group having 6 to 50 ring carbon atoms, a substituted or unsubstituted heterocyclic group having 5 to 50 ring atoms, or a group represented by the formula (H161);
at least one of a substituent, if present, for the substituted or unsubstituted monocyclic ring, a substituent, if present, for the substituted or unsubstituted fused ring, or R₁₆₀ to R₁₆₉ is a group represented by the formula (H161);
when a plurality of groups represented by the formula (H161) are present, the plurality of groups represented by the formula (H161) are mutually the same or different;
L₁₆ is a single bond, a substituted or unsubstituted arylene group having 6 to 50 ring carbon atoms, or a substituted or unsubstituted divalent heterocyclic group having 5 to 50 ring atoms;
Ar₁₆ is a substituted or unsubstituted aryl group having 6 to 50 ring carbon atoms, or a substituted or unsubstituted heterocyclic group having 5 to 50 ring atoms;
mf is 0, 1, 2, 3, 4, or 5;
when two or more L₁₆ are present, the two or more L₁₆ are mutually the same or different;
when two or more Ar₁₆ are present, the two or more Ar₁₆ are mutually the same or different; and
* in the formula (H161) represents a bonding position to a ring represented by the formula (H16).

In an exemplary arrangement of the composition of the exemplary embodiment, at least one of the first compound or the second compound is a compound represented by a formula (H162) below.

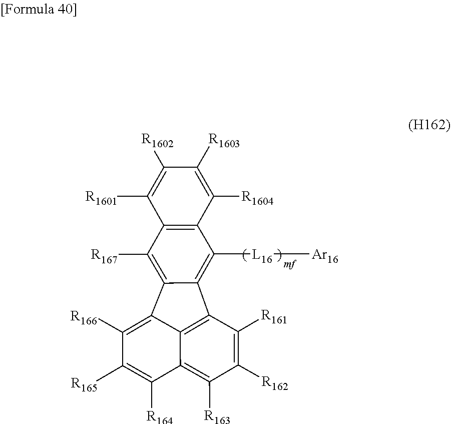

In the formula (H162):
R₁₆₁ to R₁₆₇ and R₁₆₀₁ to R₁₆₀₄ are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by—S—(R₉₀₅), a substituted or unsubstituted aralkyl group having 7 to 50 carbon atoms, a group represented by —C(═O) R₈₀₁, a group represented by —COOR₈₀₂, a halogen atom, a cyano group, a nitro group, a substituted or unsubstituted aryl group having 6 to 50 ring carbon atoms, or a substituted or unsubstituted heterocyclic group having 5 to 50 ring atoms; and
Ar₁₆, L₁₆, and mf respectively represent the same as Ar₁₆, L₁₆, and mf in the formula (H16).

In an exemplary arrangement of the composition of the exemplary embodiment, mf is 0, 1, or 2.

In an exemplary arrangement of the composition of the exemplary embodiment, the first compound and the second compound are each independently a compound selected from the group consisting of a compound represented by the formula (H111), a compound represented by the formula (H122), a compound represented by the formula (H132), and a compound represented by the formula (H133).

In an exemplary arrangement of the composition of the exemplary embodiment, each of the first compound and the second compound includes, in a molecule, neither a biscarbazole structure nor an amine structure.

The composition according to an exemplary arrangement of the exemplary embodiment contains neither a compound having a biscarbazole structure nor a compound having an amine structure.

In an exemplary arrangement of the composition according to the exemplary embodiment, the groups specified to be “substituted or unsubstituted” in the first compound and the second compound are each an “unsubstituted” group.

In an exemplary arrangement of the composition of the exemplary embodiment, the first compound and the second compound are each independently a compound represented by the formula (H11).

In an exemplary arrangement of the composition of the exemplary embodiment, the first compound and the second compound are each independently a compound represented by the formula (H111).

In an exemplary arrangement of the composition of the exemplary embodiment, the first compound is a compound represented by the formula (H11), and the second compound is a compound represented by the formula (H12).

In an exemplary arrangement of the composition of the exemplary embodiment, the first compound is a compound represented by the formula (H111), and the second compound is a compound represented by the formula (H122).

In an exemplary arrangement of the composition of the exemplary embodiment, the first compound is a compound represented by the formula (H11), and the second compound is a compound represented by the formula (H16).

In an exemplary arrangement of the composition of the exemplary embodiment, the first compound is a compound represented by the formula (H111), and the second compound is a compound represented by the formula (H162).

In an exemplary arrangement of the composition of the exemplary embodiment, the first compound is a compound represented by the formula (H13), and the second compound is a compound represented by the formula (H14).

In an exemplary arrangement of the composition of the exemplary embodiment, the first compound is a compound represented by the formula (H132), and the second compound is a compound represented by the formula (H142).

In an exemplary arrangement of the composition of the exemplary embodiment, the first compound is a compound represented by the formula (H13), and the second compound is a compound represented by the formula (H12).

In an exemplary arrangement of the composition of the exemplary embodiment, the first compound is a compound represented by the formula (H132), and the second compound is a compound represented by the formula (H122).

In an exemplary arrangement of the composition of the exemplary embodiment, the first compound is a compound represented by the formula (H132), and the second compound is a compound represented by the formula (H123).

In an exemplary arrangement of the composition of the exemplary embodiment, the first compound is a compound represented by the formula (H14), and the second compound is a compound represented by the formula (H12).

In an exemplary arrangement of the composition of the exemplary embodiment, the first compound is a compound represented by the formula (H142), and the second compound is a compound represented by the formula (H122).

In an exemplary arrangement of the composition of the exemplary embodiment, at least one of the first compound or the second compound includes, in a molecule, a ring having a hetero atom. In a compound including, in a molecule, a ring having a hetero atom, an energy level of a highest occupied molecular orbital HOMO is likely to be large.

In an exemplary arrangement of the composition of the exemplary embodiment, at least one of the first compound or the second compound includes, in a molecule, a ring having at least one of an oxygen atom or a sulfur atom.

### Method of Producing First Compound and Second Compound

The first compound and the second compound can be produced by a known method. Further, the first compound and the second compound can be produced based on a known method through a known alternative reaction using a known material(s) tailored for the target compound.

### Specific Examples of First Compound and Second Compound

Specific examples of the first compound and the second compound include compounds below. It should however be noted that the invention is not limited to the specific examples of the first compound and the second compound.

In the specific examples of the compounds herein, D represents a deuterium atom, Me represents a methyl group, tBu represents a tert-butyl group, and Ph represents a phenyl group.

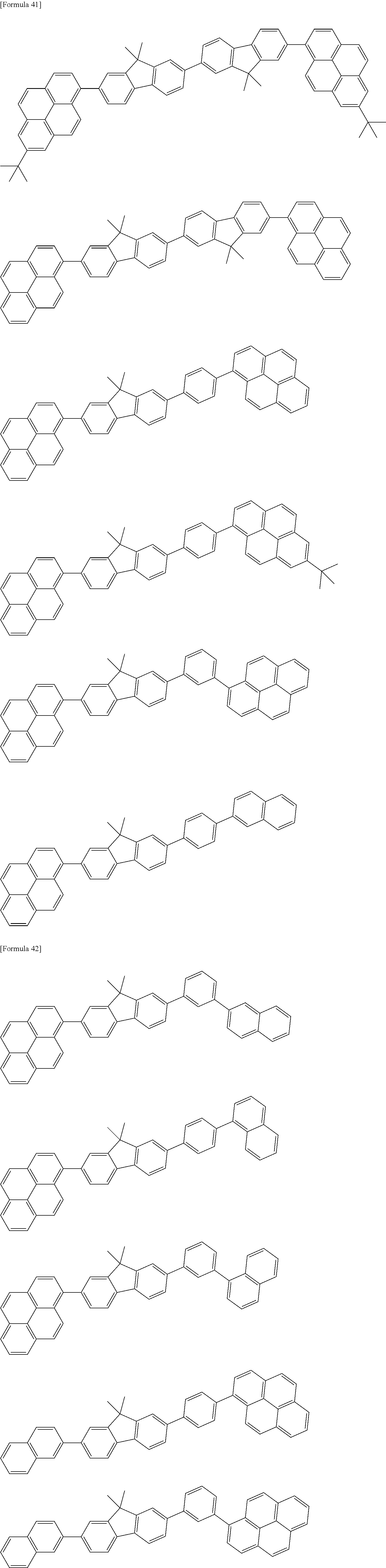

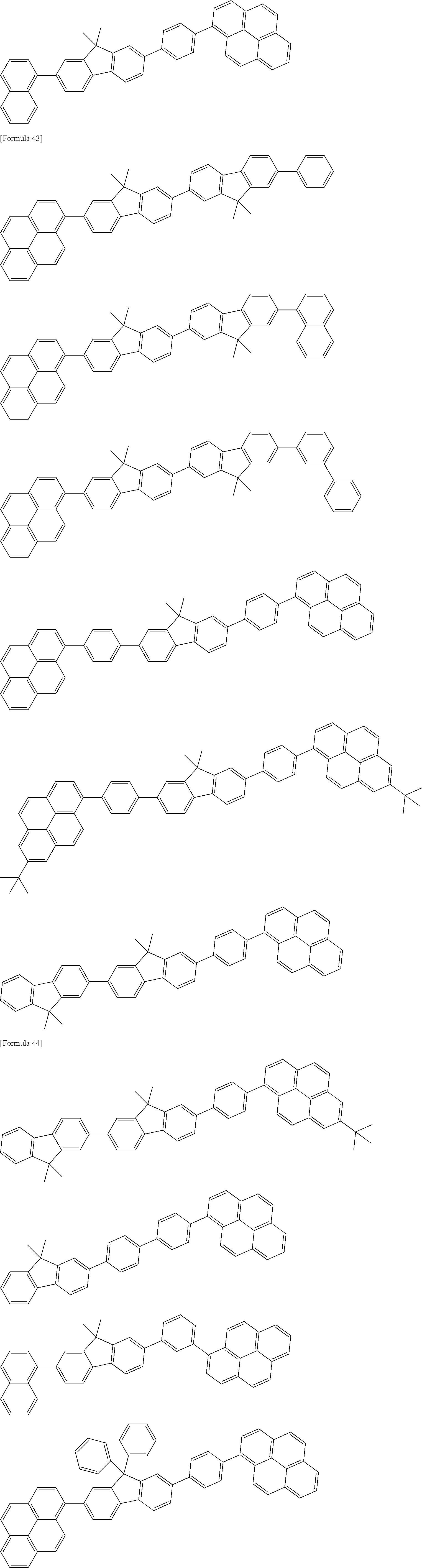

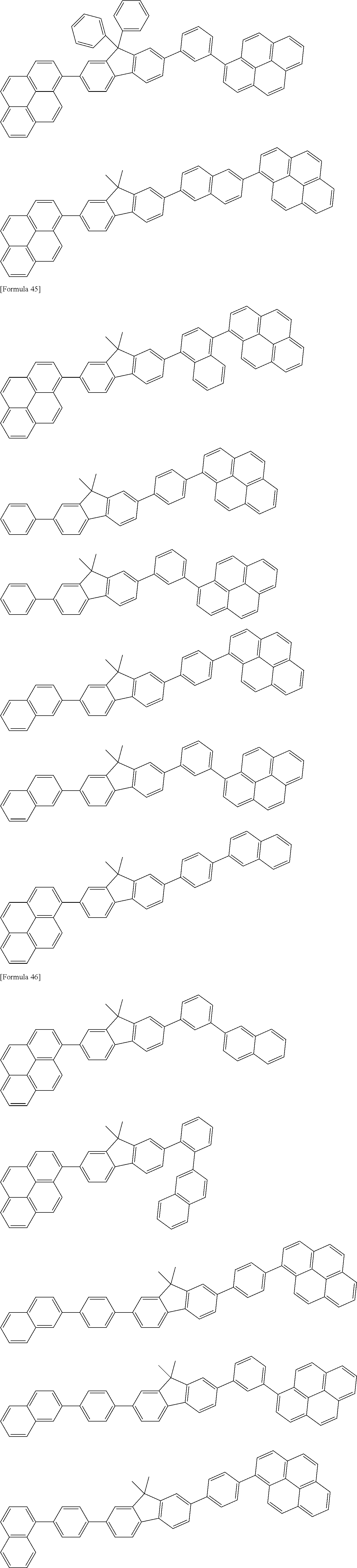

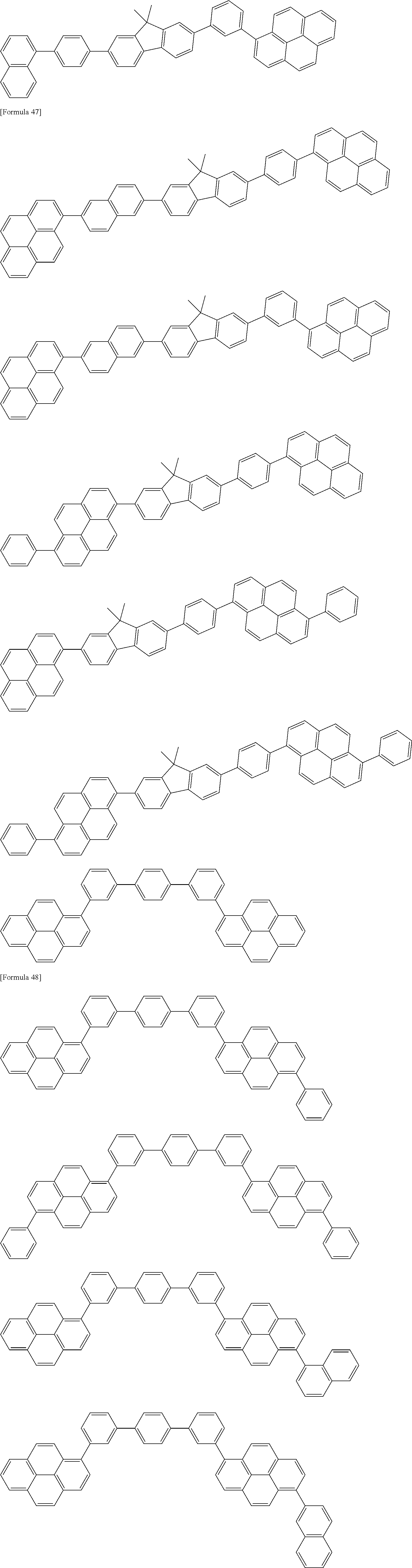

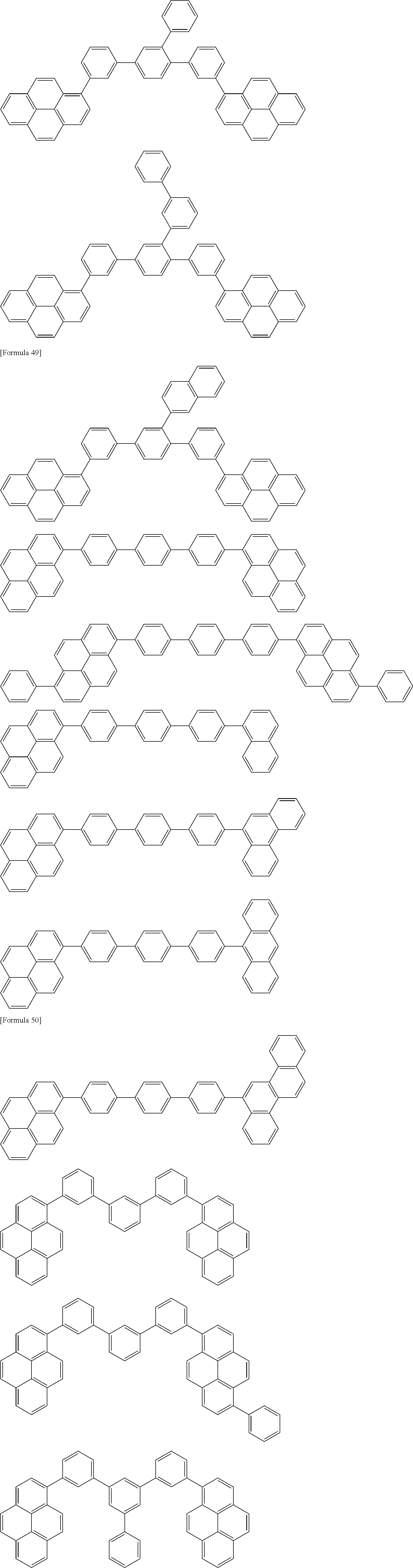

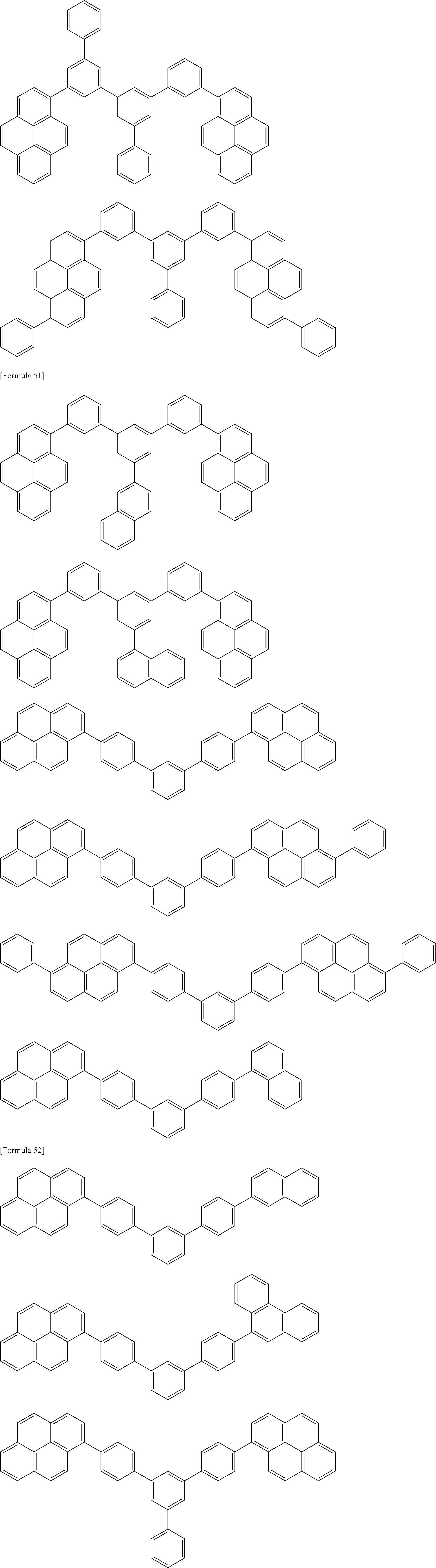

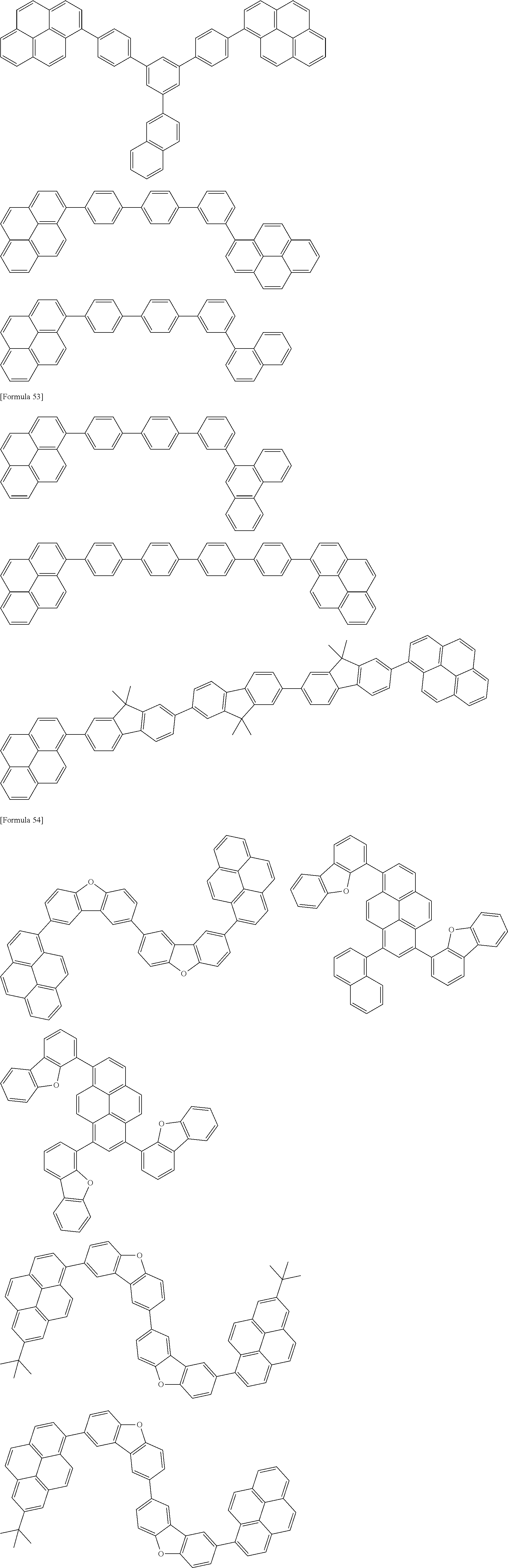

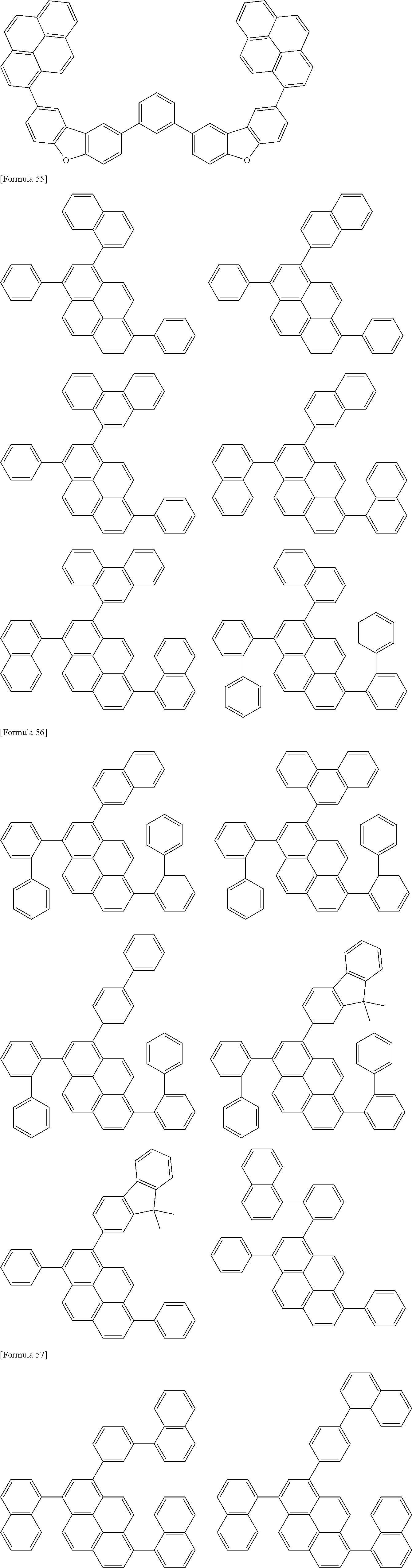

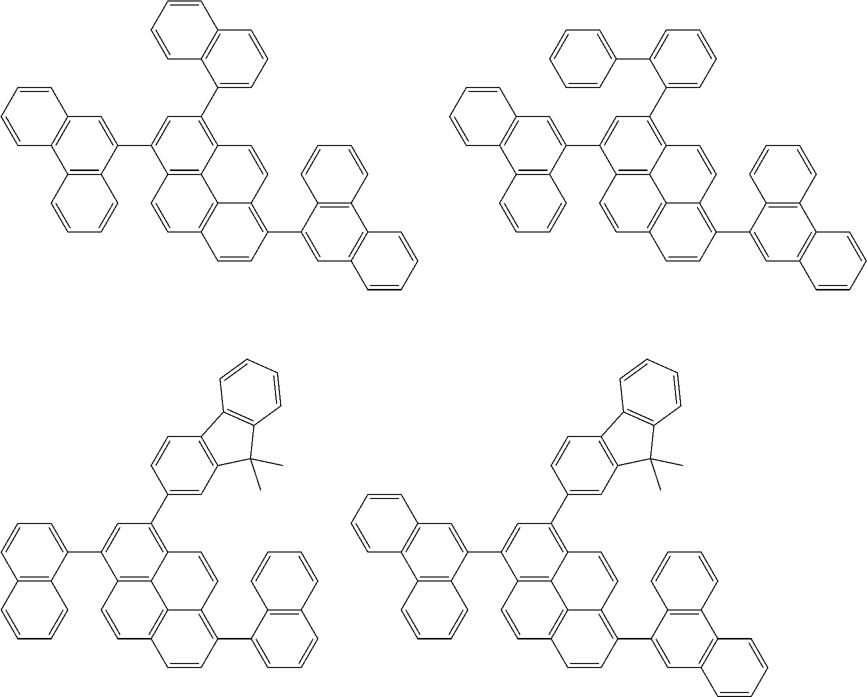

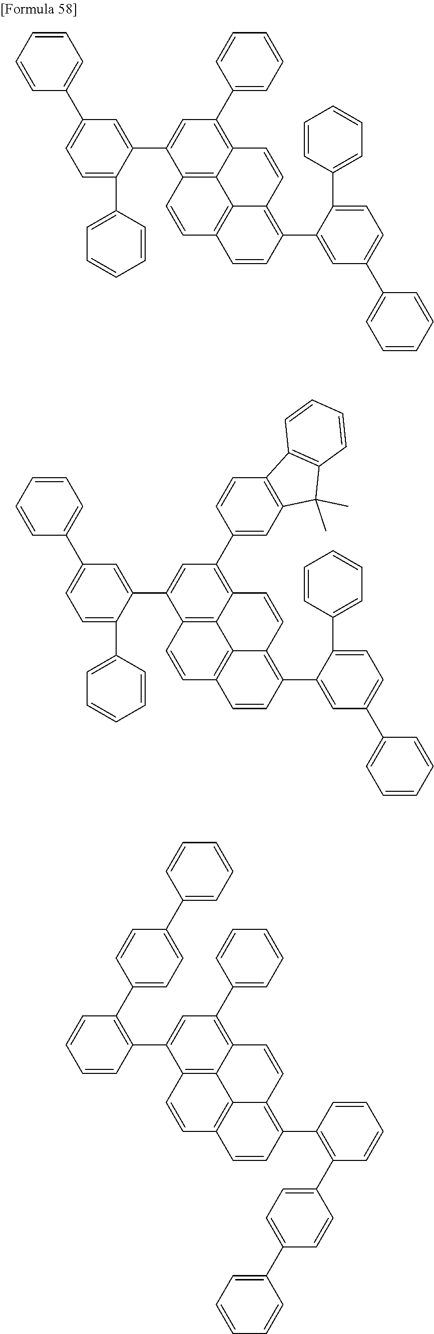

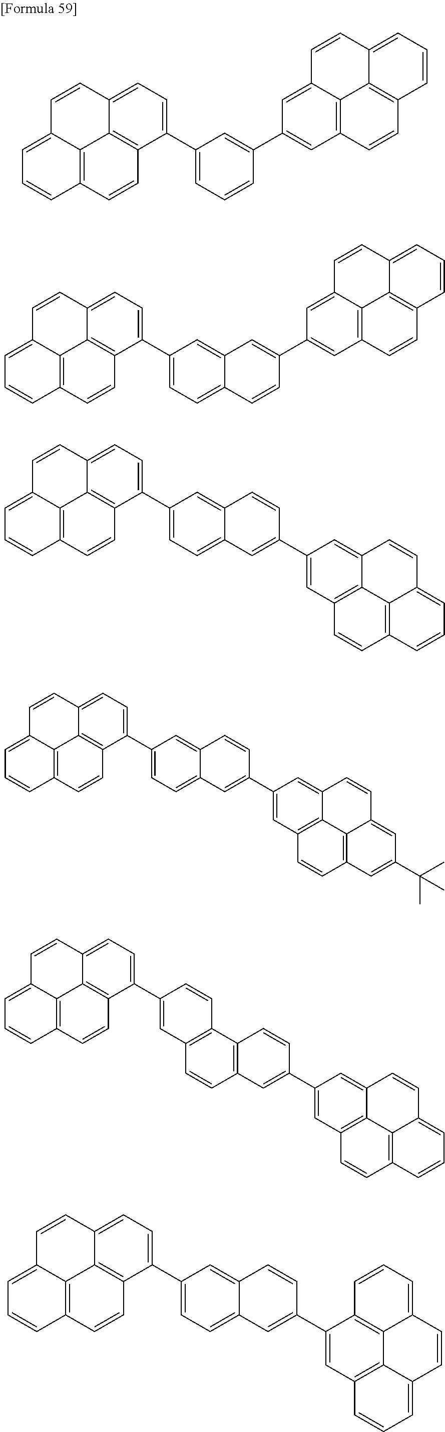

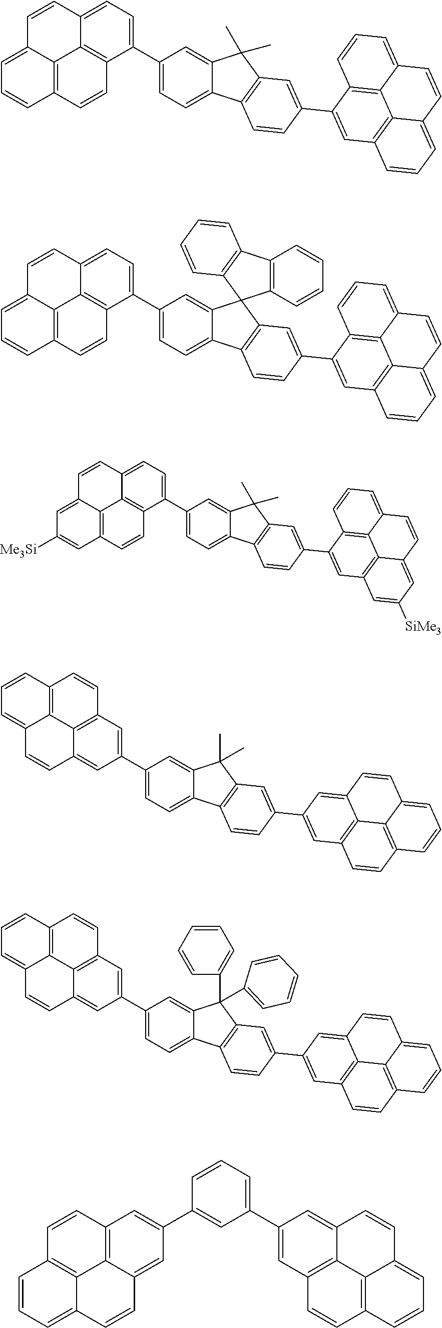

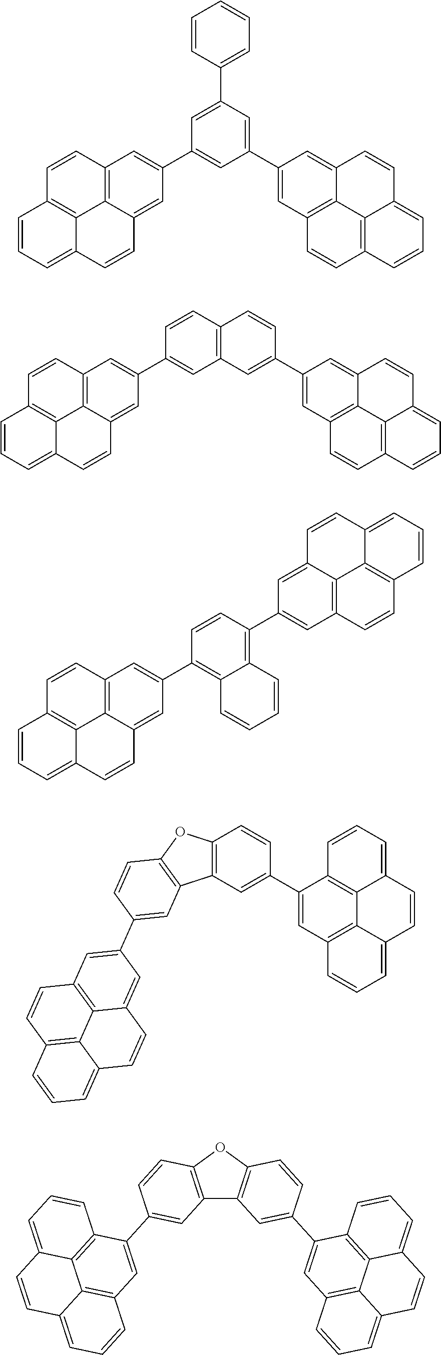

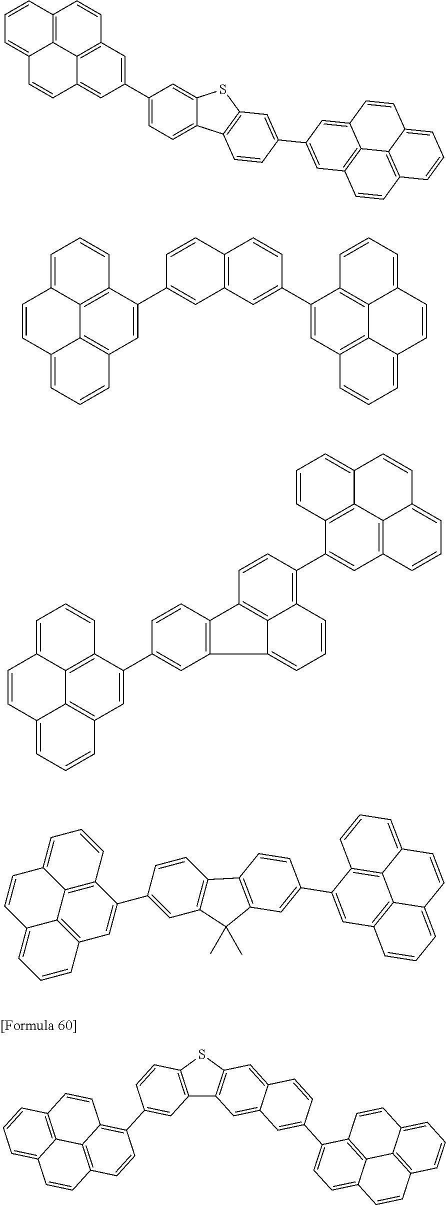

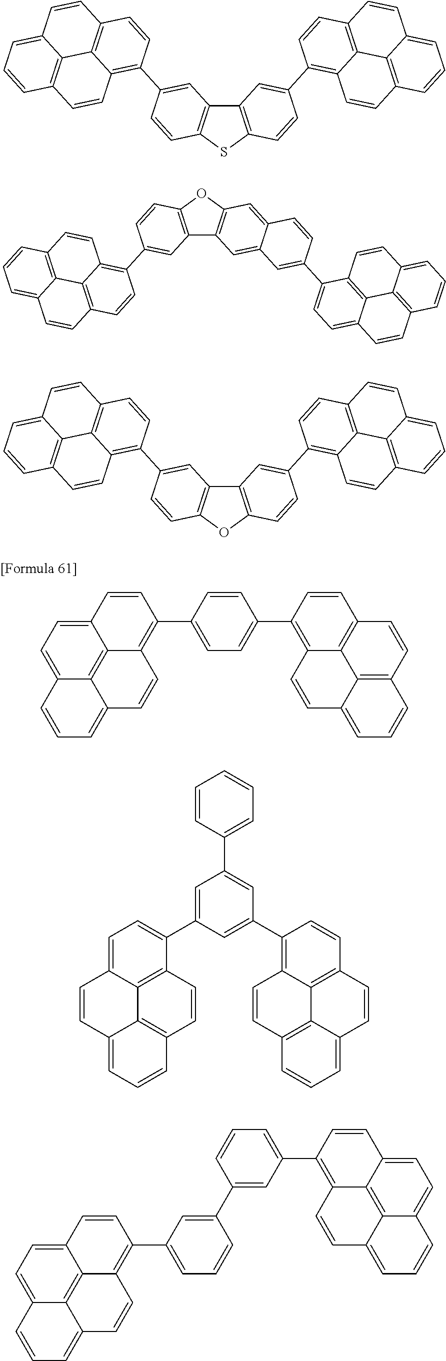

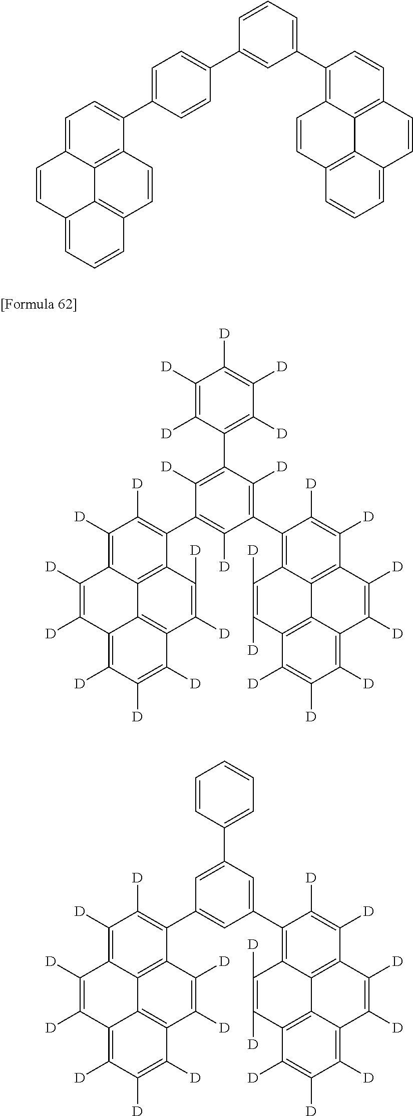

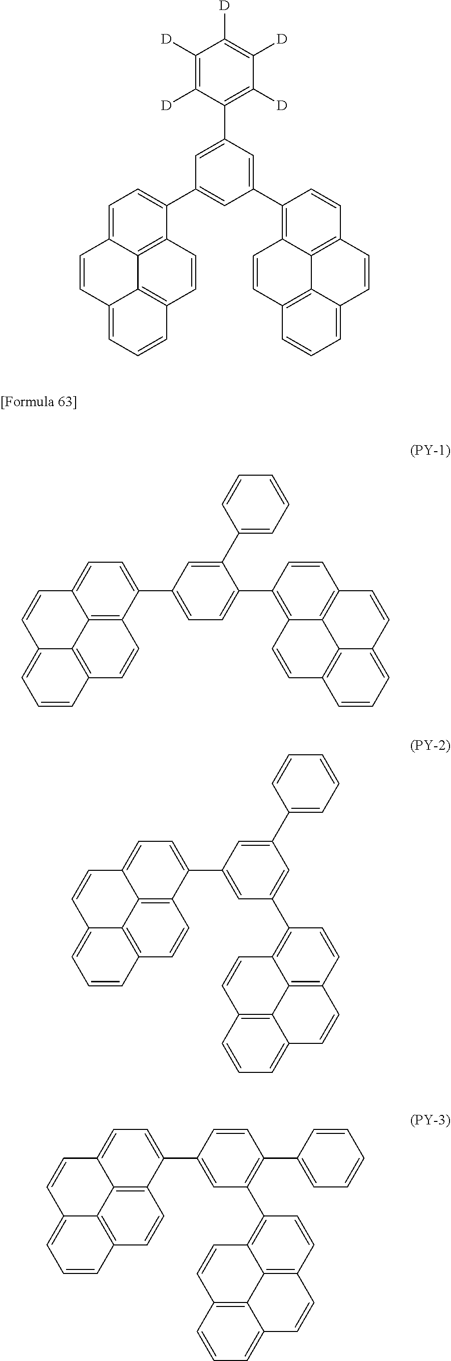

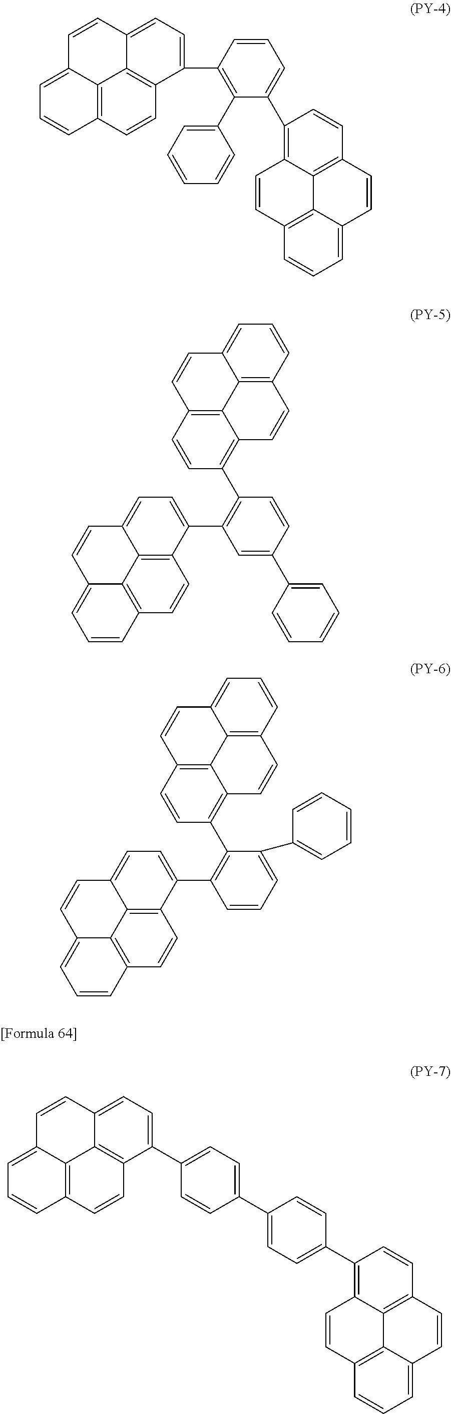

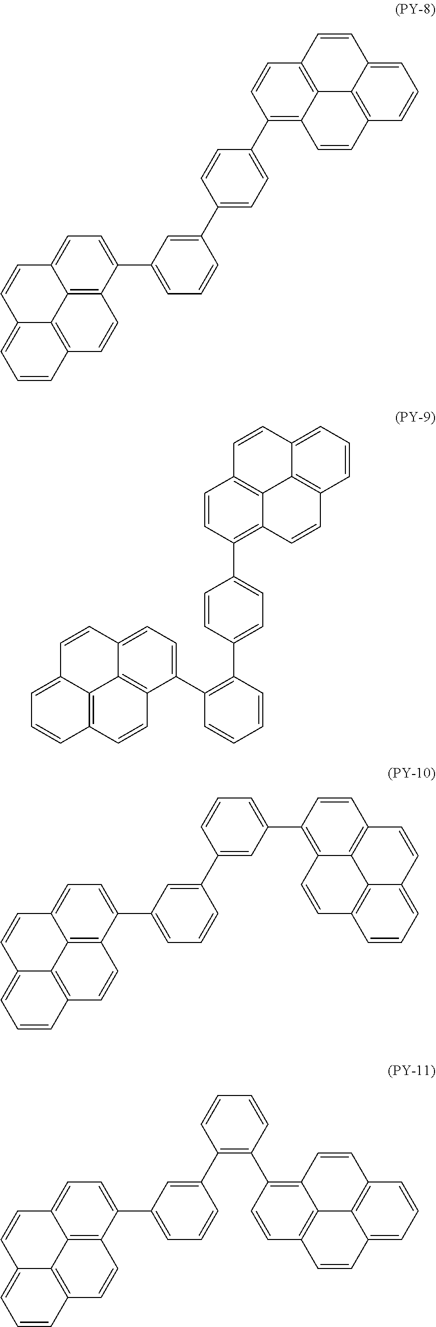

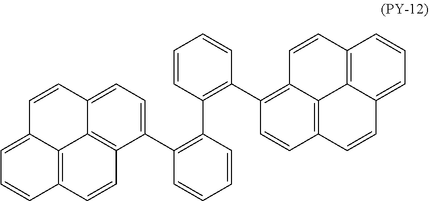 [SMILES: c1ccc(-c2ccc3ccc4cccc5ccc2c3c45)c(-c2ccccc2-c2ccc3ccc4cccc5ccc2c3c45)c1]

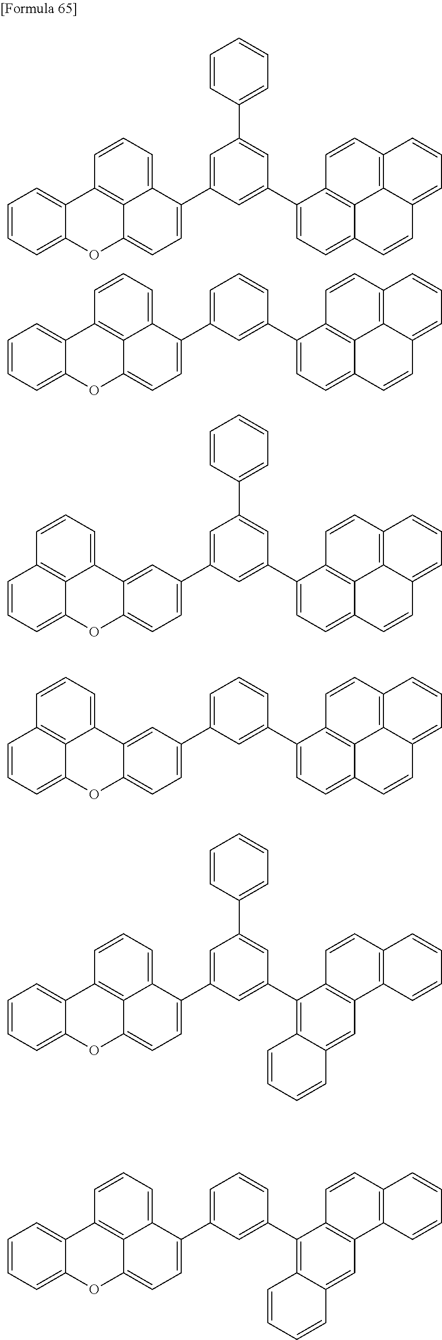

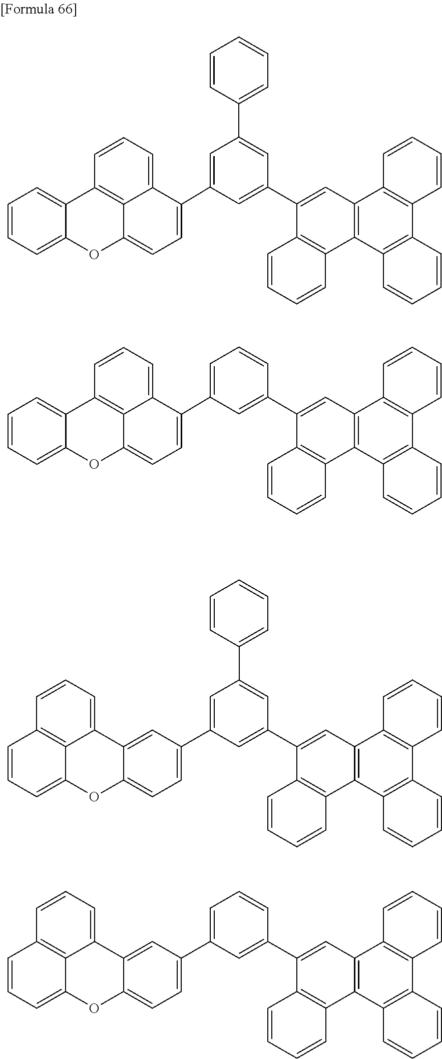

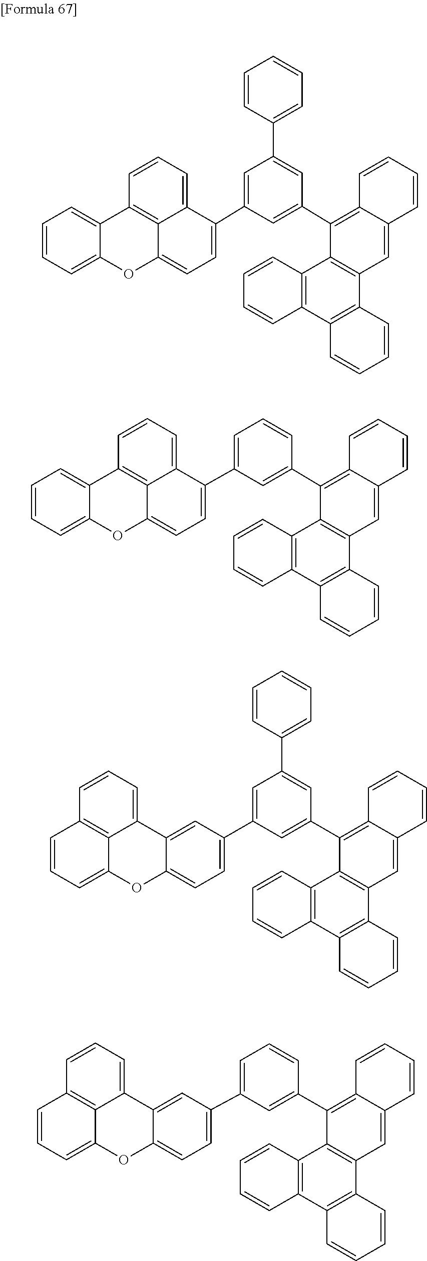 [SMILES: c1ccc(-c2cc(-c3ccc4c5c(cccc35)-c3ccccc3O4)cc(-c3c4ccccc4cc4c5ccccc5c5ccccc5c34)c2)cc1 ; c1cc(-c2ccc3c4c(cccc24)-c2ccccc2O3)cc(-c2c3ccccc3cc3c4ccccc4c4ccccc4c23)c1 ; c1ccc(-c2cc(-c3ccc4c(c3)-c3cccc5cccc(c35)O4)cc(-c3c4ccccc4cc4c5ccccc5c5ccccc5c34)c2)cc1 ; c1cc(-c2ccc3c(c2)-c2cccc4cccc(c24)O3)cc(-c2c3ccccc3cc3c4ccccc4c4ccccc4c23)c1]

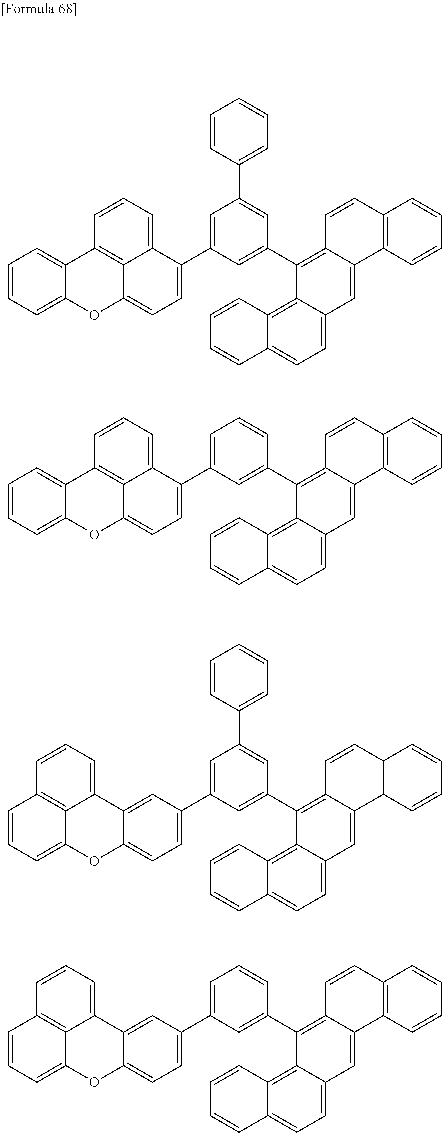 [SMILES: c1ccc(-c2cc(-c3ccc4c5c(cccc35)-c3ccccc3O4)cc(-c3c4ccc5ccccc5c4cc4ccc5ccccc5c34)c2)cc1 ; c1cc(-c2ccc3c4c(cccc24)-c2ccccc2O3)cc(-c2c3ccc4ccccc4c3cc3ccc4ccccc4c23)c1 ; C1=CC2C=Cc3c(cc4ccc5ccccc5c4c3-c3cc(-c4ccccc4)cc(-c4ccc5c(c4)-c4cccc6cccc(c46)O5)c3)C2C=C1 ; c1cc(-c2ccc3c(c2)-c2cccc4cccc(c24)O3)cc(-c2c3ccc4ccccc4c3cc3ccc4ccccc4c23)c1]

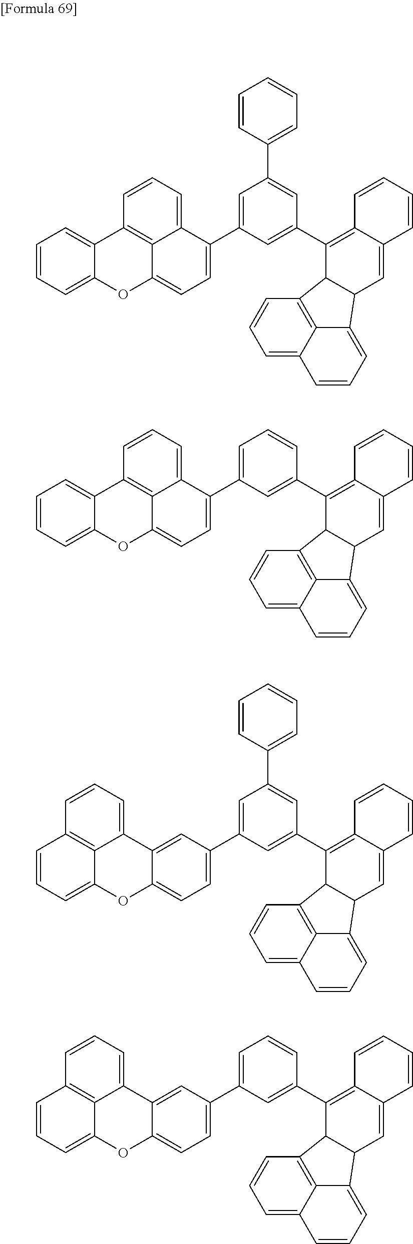 [SMILES: C1=c2ccccc2=C(c2cc(-c3ccccc3)cc(-c3ccc4c5c(cccc35)-c3ccccc3O4)c2)C2c3cccc4cccc(c34)C12 ; C1=c2ccccc2=C(c2cccc(-c3ccc4c5c(cccc35)-c3ccccc3O4)c2)C2c3cccc4cccc(c34)C12 ; C1=c2ccccc2=C(c2cc(-c3ccccc3)cc(-c3ccc4c(c3)-c3cccc5cccc(c35)O4)c2)C2c3cccc4cccc(c34)C12 ; C1=c2ccccc2=C(c2cccc(-c3ccc4c(c3)-c3cccc5cccc(c35)O4)c2)C2c3cccc4cccc(c34)C12]

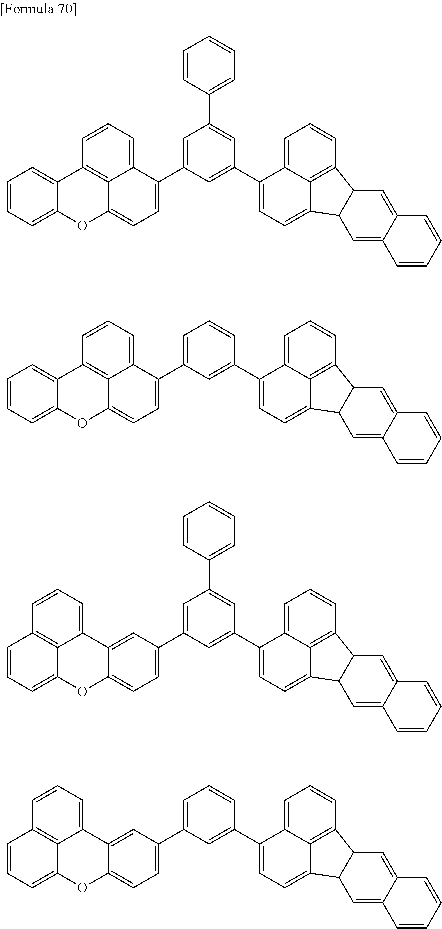 [SMILES: C1=c2ccccc2=CC2c3ccc(-c4cc(-c5ccccc5)cc(-c5ccc6c7c(cccc57)-c5ccccc5O6)c4)c4cccc(c34)C12 ; C1=c2ccccc2=CC2c3ccc(-c4cccc(-c5ccc6c7c(cccc57)-c5ccccc5O6)c4)c4cccc(c34)C12 ; C1=c2ccccc2=CC2c3ccc(-c4cc(-c5ccccc5)cc(-c5ccc6c(c5)-c5cccc7cccc(c57)O6)c4)c4cccc(c34)C12 ; C1=c2ccccc2=CC2c3ccc(-c4cccc(-c5ccc6c(c5)-c5cccc7cccc(c57)O6)c4)c4cccc(c34)C12]

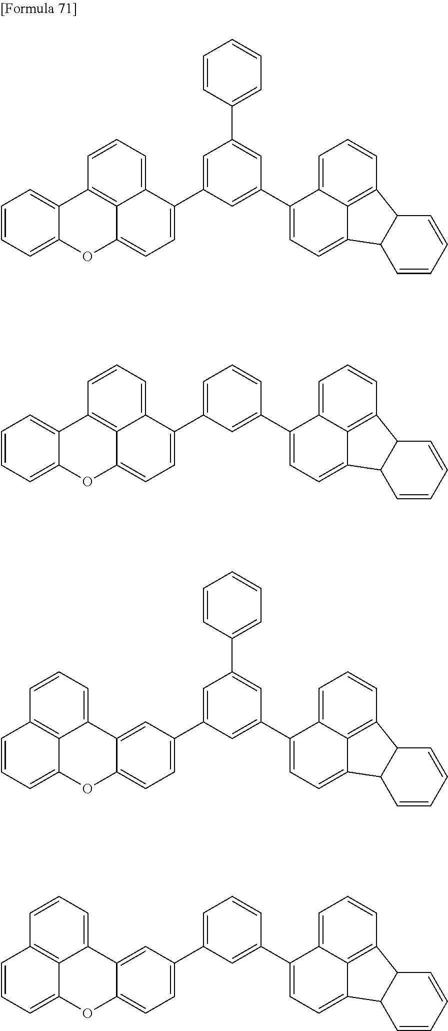 [SMILES: C1=CC2c3cccc4c(-c5cc(-c6ccccc6)cc(-c6ccc7c8c(cccc68)-c6ccccc6O7)c5)ccc(c34)C2C=C1 ; C1=CC2c3cccc4c(-c5cccc(-c6ccc7c8c(cccc68)-c6ccccc6O7)c5)ccc(c34)C2C=C1 ; C1=CC2c3cccc4c(-c5cc(-c6ccccc6)cc(-c6ccc7c(c6)-c6cccc8cccc(c68)O7)c5)ccc(c34)C2C=C1 ; C1=CC2c3cccc4c(-c5cccc(-c6ccc7c(c6)-c6cccc8cccc(c68)O7)c5)ccc(c34)C2C=C1]

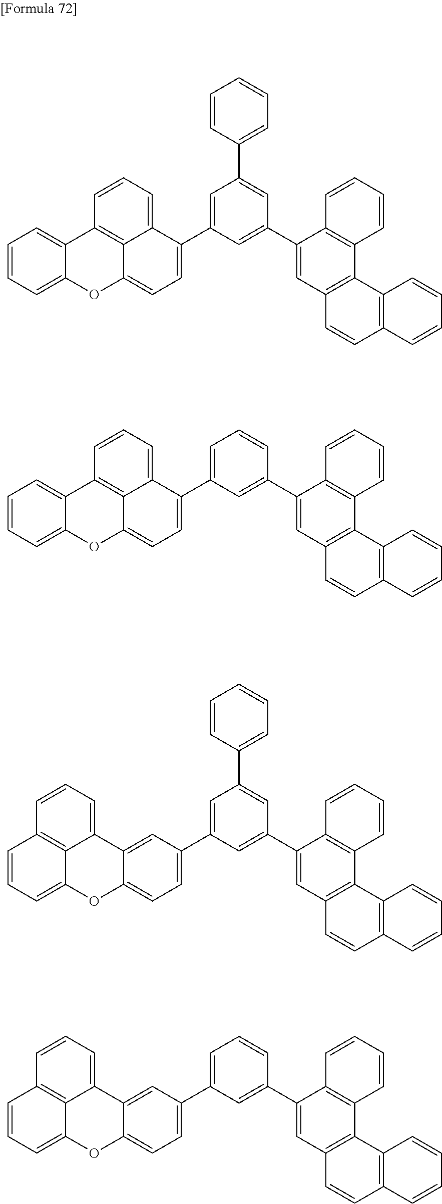 [SMILES: c1ccc(-c2cc(-c3ccc4c5c(cccc35)-c3ccccc3O4)cc(-c3cc4ccc5ccccc5c4c4ccccc34)c2)cc1 ; c1cc(-c2ccc3c4c(cccc24)-c2ccccc2O3)cc(-c2cc3ccc4ccccc4c3c3ccccc23)c1 ; c1ccc(-c2cc(-c3ccc4c(c3)-c3cccc5cccc(c35)O4)cc(-c3cc4ccc5ccccc5c4c4ccccc34)c2)cc1 ; c1cc(-c2ccc3c(c2)-c2cccc4cccc(c24)O3)cc(-c2cc3ccc4ccccc4c3c3ccccc23)c1]

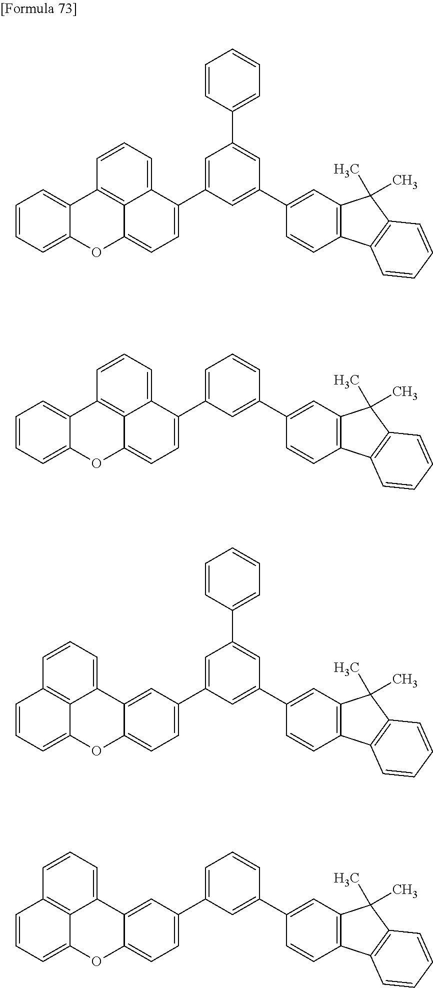 [SMILES: CC1(C)c2ccccc2-c2ccc(-c3cc(-c4ccccc4)cc(-c4ccc5c6c(cccc46)-c4ccccc4O5)c3)cc21 ; CC1(C)c2ccccc2-c2ccc(-c3cccc(-c4ccc5c6c(cccc46)-c4ccccc4O5)c3)cc21 ; CC1(C)c2ccccc2-c2ccc(-c3cc(-c4ccccc4)cc(-c4ccc5c(c4)-c4cccc6cccc(c46)O5)c3)cc21 ; CC1(C)c2ccccc2-c2ccc(-c3cccc(-c4ccc5c(c4)-c4cccc6cccc(c46)O5)c3)cc21]

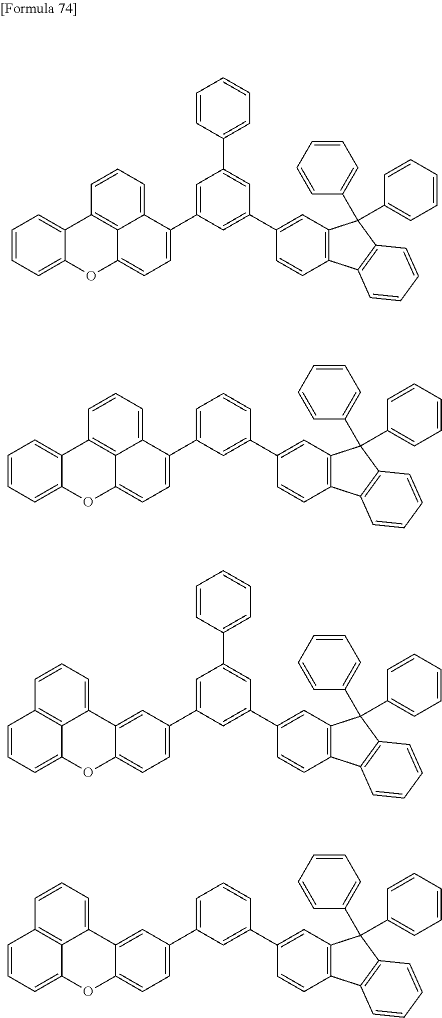 [SMILES: c1ccc(-c2cc(-c3ccc4c(c3)C(c3ccccc3)(c3ccccc3)c3ccccc3-4)cc(-c3ccc4c5c(cccc35)-c3ccccc3O4)c2)cc1 ; c1ccc(C2(c3ccccc3)c3ccccc3-c3ccc(-c4cccc(-c5ccc6c7c(cccc57)-c5ccccc5O6)c4)cc32)cc1 ; c1ccc(-c2cc(-c3ccc4c(c3)-c3cccc5cccc(c35)O4)cc(-c3ccc4c(c3)C(c3ccccc3)(c3ccccc3)c3ccccc3-4)c2)cc1 ; c1ccc(C2(c3ccccc3)c3ccccc3-c3ccc(-c4cccc(-c5ccc6c(c5)-c5cccc7cccc(c57)O6)c4)cc32)cc1]

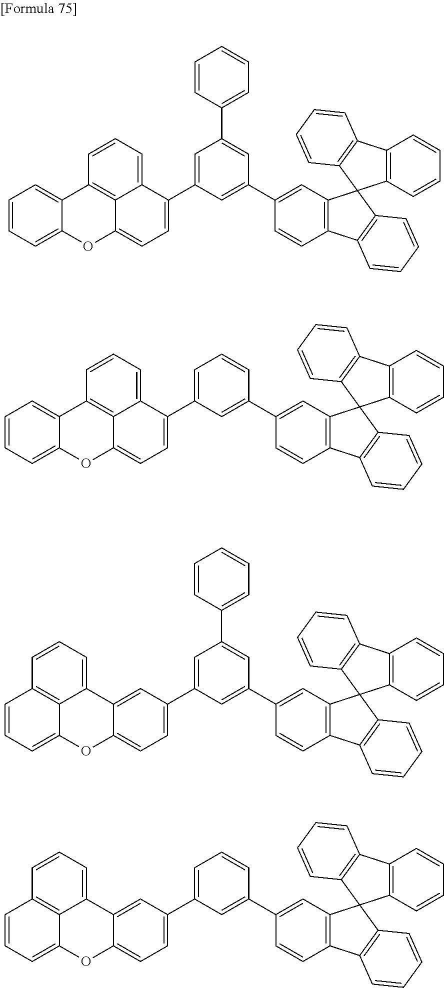 [SMILES: c1ccc(-c2cc(-c3ccc4c(c3)C3(c5ccccc5-c5ccccc53)c3ccccc3-4)cc(-c3ccc4c5c(cccc35)-c3ccccc3O4)c2)cc1 ; c1cc(-c2ccc3c(c2)C2(c4ccccc4-c4ccccc42)c2ccccc2-3)cc(-c2ccc3c4c(cccc24)-c2ccccc2O3)c1 ; c1ccc(-c2cc(-c3ccc4c(c3)-c3cccc5cccc(c35)O4)cc(-c3ccc4c(c3)C3(c5ccccc5-c5ccccc53)c3ccccc3-4)c2)cc1 ; c1cc(-c2ccc3c(c2)-c2cccc4cccc(c24)O3)cc(-c2ccc3c(c2)C2(c4ccccc4-c4ccccc42)c2ccccc2-3)c1]

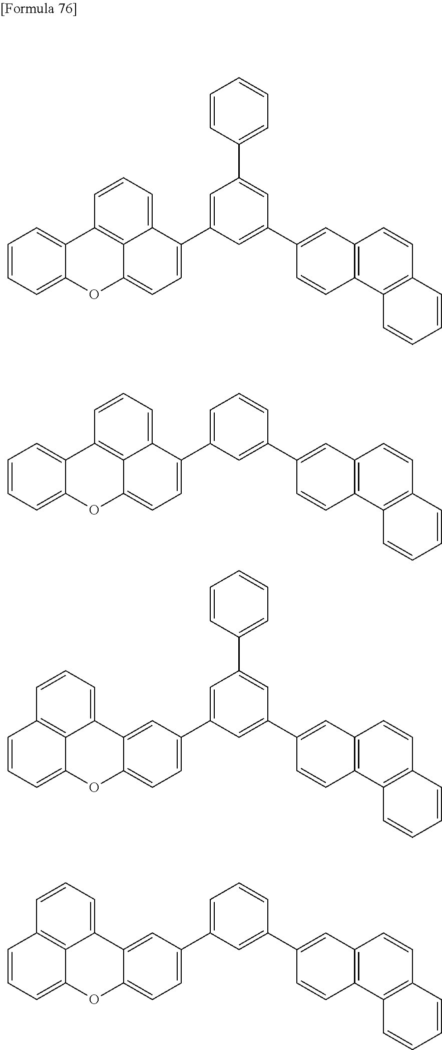 [SMILES: c1ccc(-c2cc(-c3ccc4c(ccc5ccccc54)c3)cc(-c3ccc4c5c(cccc35)-c3ccccc3O4)c2)cc1 ; c1cc(-c2ccc3c(ccc4ccccc43)c2)cc(-c2ccc3c4c(cccc24)-c2ccccc2O3)c1 ; c1ccc(-c2cc(-c3ccc4c(c3)-c3cccc5cccc(c35)O4)cc(-c3ccc4c(ccc5ccccc54)c3)c2)cc1 ; c1cc(-c2ccc3c(c2)-c2cccc4cccc(c24)O3)cc(-c2ccc3c(ccc4ccccc43)c2)c1]

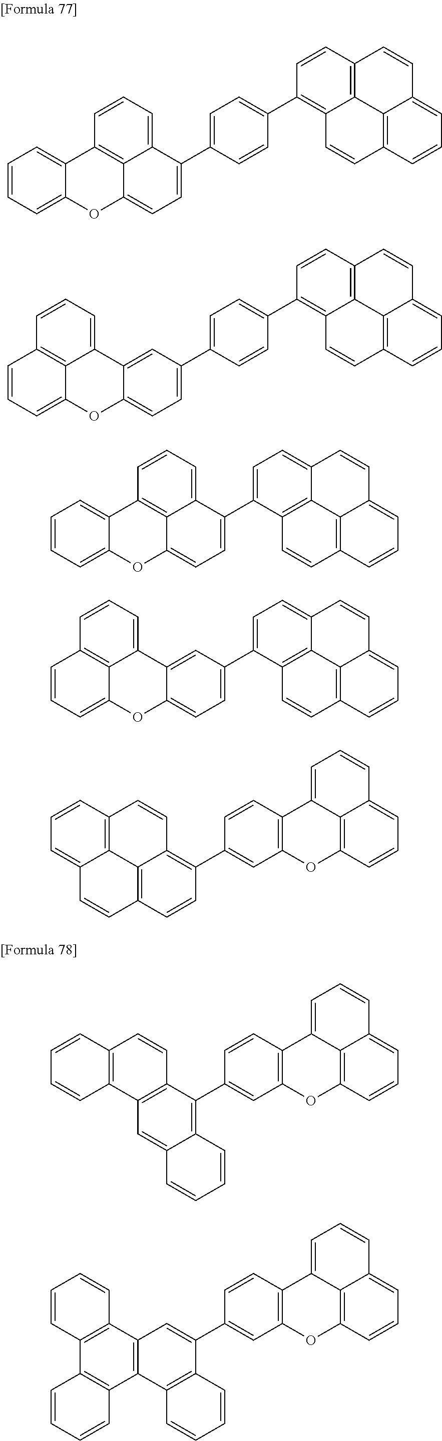

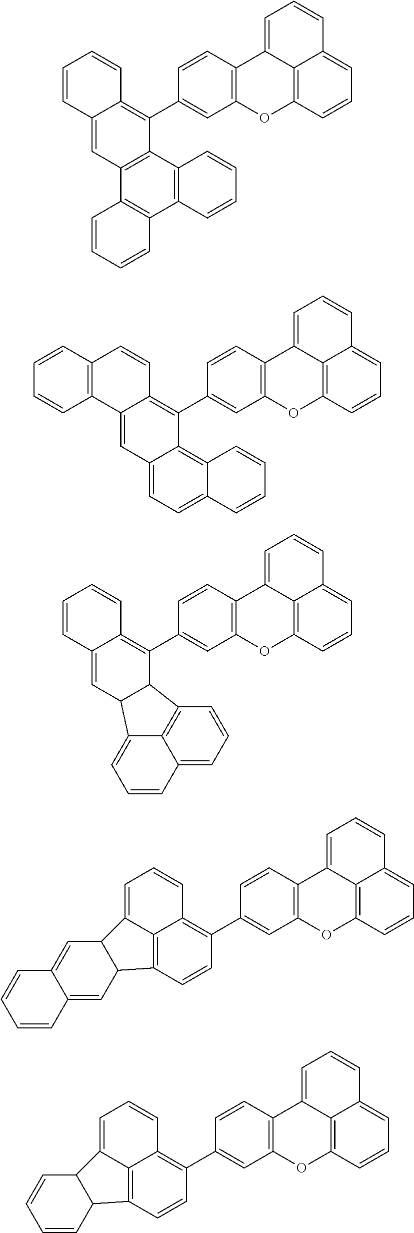 [SMILES: c1ccc2c(-c3ccc4c(c3)Oc3cccc5cccc-4c35)c3c4ccccc4c4ccccc4c3cc2c1 ; c1cc2c3c(cccc3c1)-c1ccc(-c3c4ccc5ccccc5c4cc4ccc5ccccc5c34)cc1O2 ; C1=c2ccccc2=C(c2ccc3c(c2)Oc2cccc4cccc-3c24)C2c3cccc4cccc(c34)C12 ; C1=c2ccccc2=CC2c3ccc(-c4ccc5c(c4)Oc4cccc6cccc-5c46)c4cccc(c34)C12 ; C1=CC2c3cccc4c(-c5ccc6c(c5)Oc5cccc7cccc-6c57)ccc(c34)C2C=C1]

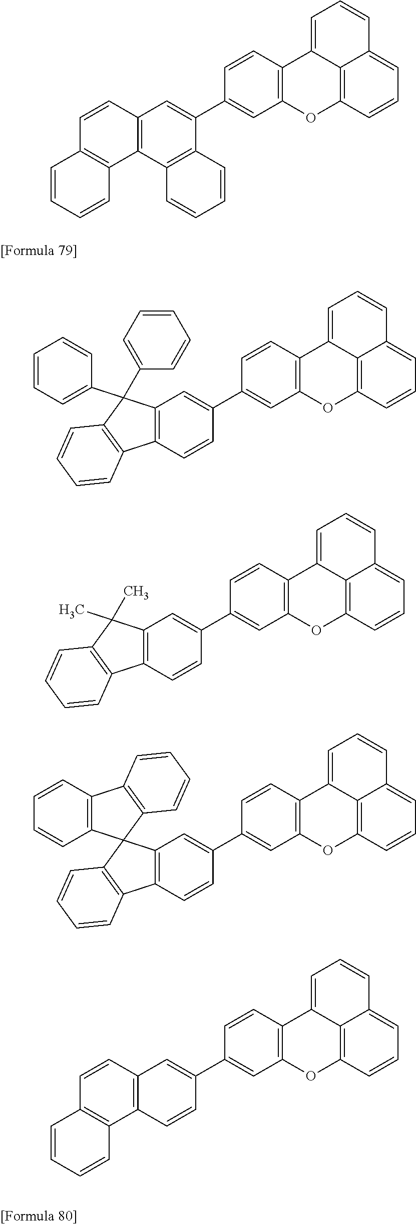 [SMILES: c1cc2c3c(cccc3c1)-c1ccc(-c3cc4ccc5ccccc5c4c4ccccc34)cc1O2 ; c1ccc(C2(c3ccccc3)c3ccccc3-c3ccc(-c4ccc5c(c4)Oc4cccc6cccc-5c46)cc32)cc1 ; CC1(C)c2ccccc2-c2ccc(-c3ccc4c(c3)Oc3cccc5cccc-4c35)cc21 ; c1ccc2c(c1)-c1ccccc1C21c2ccccc2-c2ccc(-c3ccc4c(c3)Oc3cccc5cccc-4c35)cc21 ; c1cc2c3c(cccc3c1)-c1ccc(-c3ccc4c(ccc5ccccc54)c3)cc1O2]

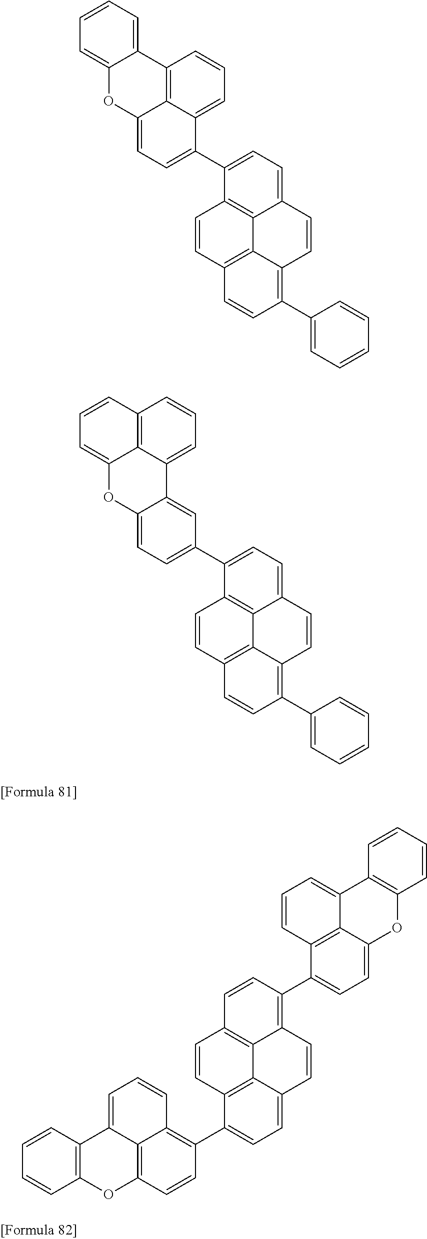 [SMILES: c1ccc(-c2ccc3ccc4c(-c5ccc6c7c(cccc57)-c5ccccc5O6)ccc5ccc2c3c54)cc1 ; c1ccc(-c2ccc3ccc4c(-c5ccc6c(c5)-c5cccc7cccc(c57)O6)ccc5ccc2c3c54)cc1 ; c1ccc2c(c1)Oc1ccc(-c3ccc4ccc5c(-c6ccc7c8c(cccc68)-c6ccccc6O7)ccc6ccc3c4c65)c3cccc-2c13]

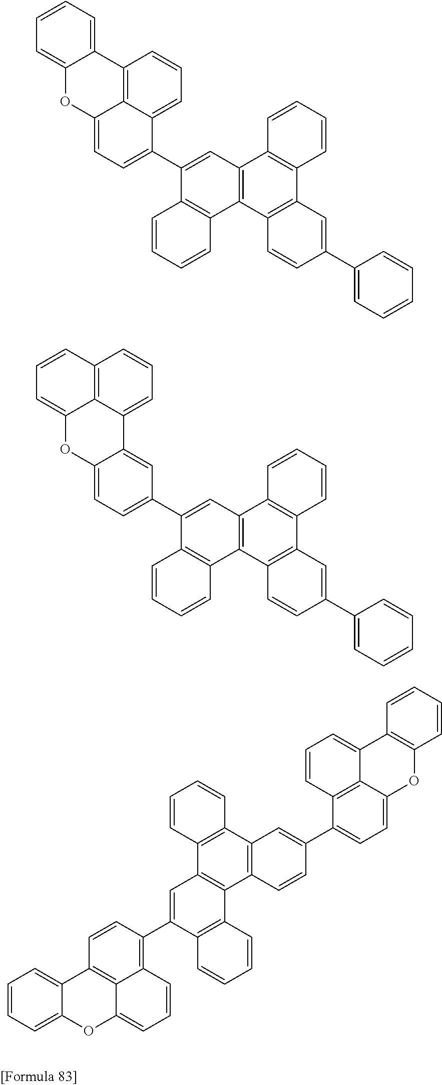

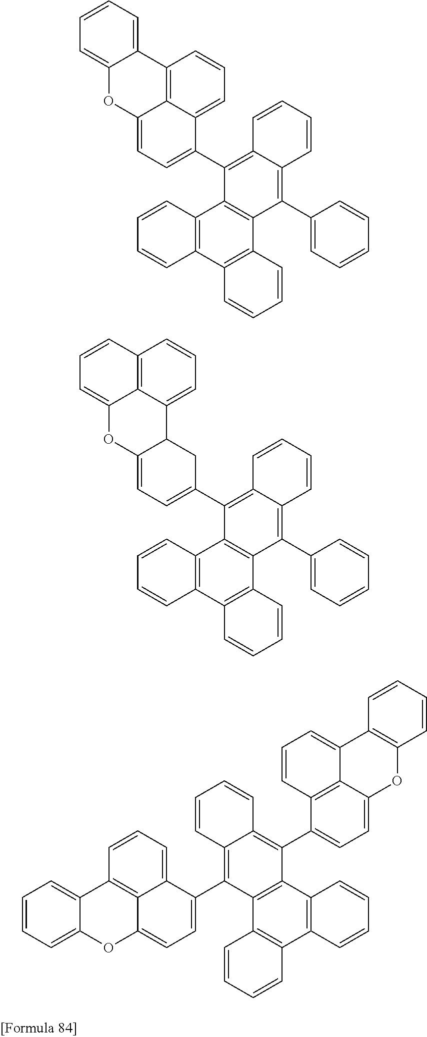 [SMILES: c1ccc(-c2c3ccccc3c(-c3ccc4c5c(cccc35)-c3ccccc3O4)c3c4ccccc4c4ccccc4c23)cc1 ; C1=C(c2c3ccccc3c(-c3ccccc3)c3c4ccccc4c4ccccc4c23)CC2C(=C1)Oc1cccc3cccc2c13 ; c1ccc2c(c1)Oc1ccc(-c3c4ccccc4c(-c4ccc5c6c(cccc46)-c4ccccc4O5)c4c5ccccc5c5ccccc5c34)c3cccc-2c13]

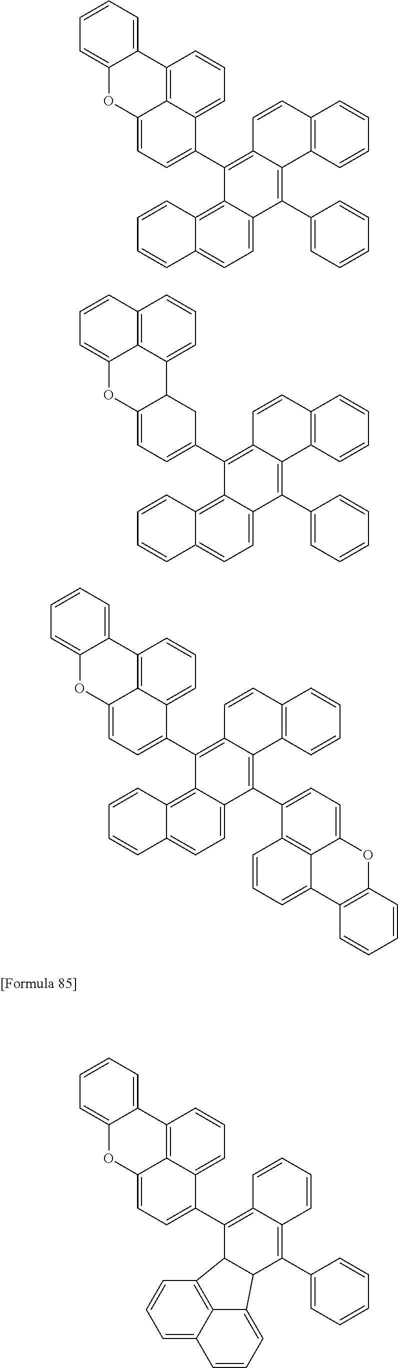

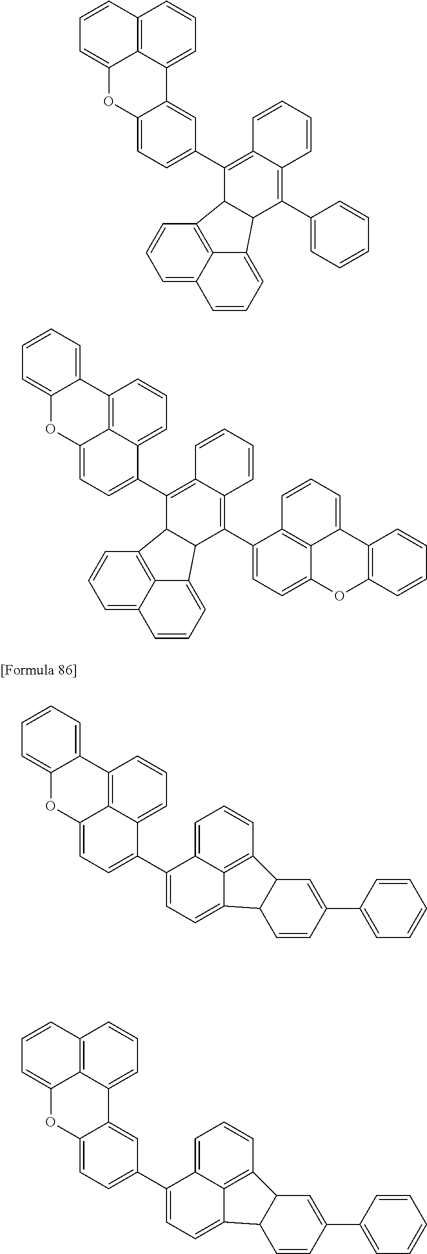 [SMILES: c1ccc(C2=c3ccccc3=C(c3ccc4c(c3)-c3cccc5cccc(c35)O4)C3c4cccc5cccc(c45)C23)cc1 ; c1ccc2c(c1)Oc1ccc(C3=c4ccccc4=C(c4ccc5c6c(cccc46)-c4ccccc4O5)C4c5cccc6cccc(c56)C34)c3cccc-2c13 ; C1=CC2c3ccc(-c4ccc5c6c(cccc46)-c4ccccc4O5)c4cccc(c34)C2C=C1c1ccccc1 ; C1=CC2c3ccc(-c4ccc5c(c4)-c4cccc6cccc(c46)O5)c4cccc(c34)C2C=C1c1ccccc1]

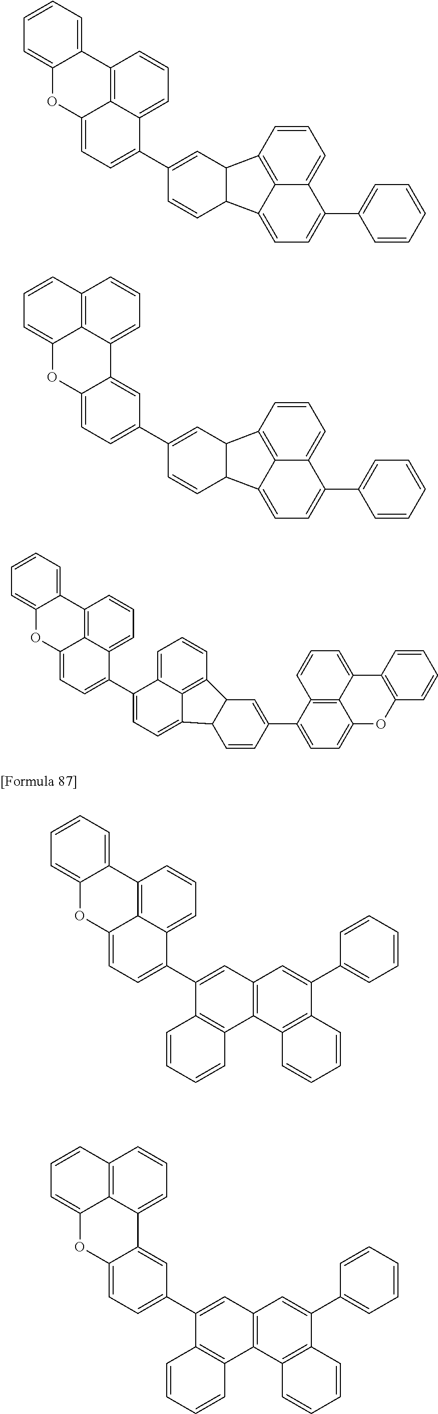 [SMILES: C1=CC2c3ccc(-c4ccccc4)c4cccc(c34)C2C=C1c1ccc2c3c(cccc13)-c1ccccc1O2 ; C1=CC2c3ccc(-c4ccccc4)c4cccc(c34)C2C=C1c1ccc2c(c1)-c1cccc3cccc(c13)O2 ; C1=CC2c3ccc(-c4ccc5c6c(cccc46)-c4ccccc4O5)c4cccc(c34)C2C=C1c1ccc2c3c(cccc13)-c1ccccc1O2 ; c1ccc(-c2cc3cc(-c4ccc5c6c(cccc46)-c4ccccc4O5)c4ccccc4c3c3ccccc23)cc1 ; c1ccc(-c2cc3cc(-c4ccc5c(c4)-c4cccc6cccc(c46)O5)c4ccccc4c3c3ccccc23)cc1]

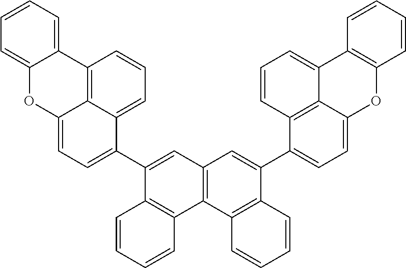 [SMILES: c1ccc2c(c1)Oc1ccc(-c3cc4cc(-c5ccc6c7c(cccc57)-c5ccccc5O6)c5ccccc5c4c4ccccc34)c3cccc-2c13]

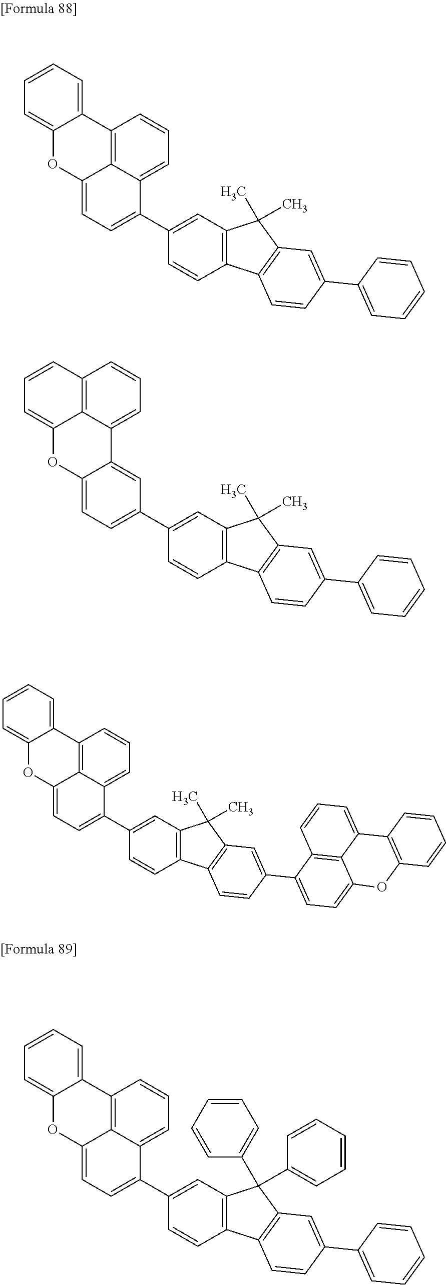 [SMILES: CC1(C)c2cc(-c3ccccc3)ccc2-c2ccc(-c3ccc4c5c(cccc35)-c3ccccc3O4)cc21 ; CC1(C)c2cc(-c3ccccc3)ccc2-c2ccc(-c3ccc4c(c3)-c3cccc5cccc(c35)O4)cc21 ; CC1(C)c2cc(-c3ccc4c5c(cccc35)-c3ccccc3O4)ccc2-c2ccc(-c3ccc4c5c(cccc35)-c3ccccc3O4)cc21 ; c1ccc(-c2ccc3c(c2)C(c2ccccc2)(c2ccccc2)c2cc(-c4ccc5c6c(cccc46)-c4ccccc4O5)ccc2-3)cc1]

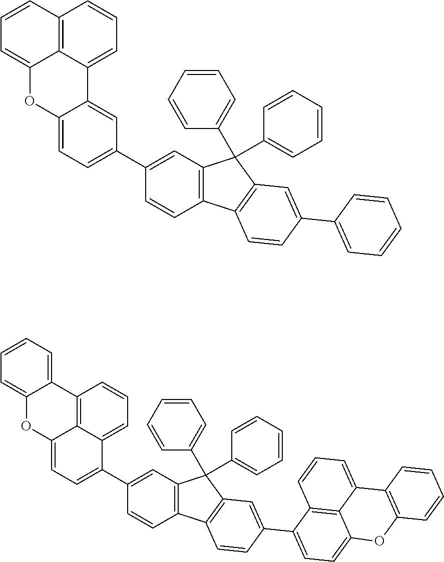

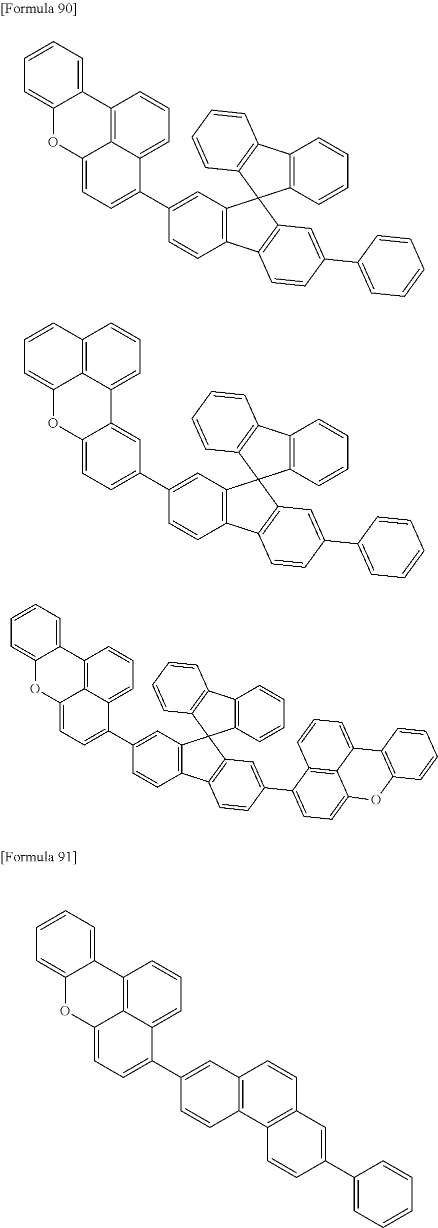 [SMILES: c1ccc(-c2ccc3c(c2)C2(c4ccccc4-c4ccccc42)c2cc(-c4ccc5c6c(cccc46)-c4ccccc4O5)ccc2-3)cc1 ; c1ccc(-c2ccc3c(c2)C2(c4ccccc4-c4ccccc42)c2cc(-c4ccc5c(c4)-c4cccc6cccc(c46)O5)ccc2-3)cc1 ; c1ccc2c(c1)Oc1ccc(-c3ccc4c(c3)C3(c5ccccc5-c5ccccc53)c3cc(-c5ccc6c7c(cccc57)-c5ccccc5O6)ccc3-4)c3cccc-2c13 ; c1ccc(-c2ccc3c(ccc4cc(-c5ccc6c7c(cccc57)-c5ccccc5O6)ccc43)c2)cc1]

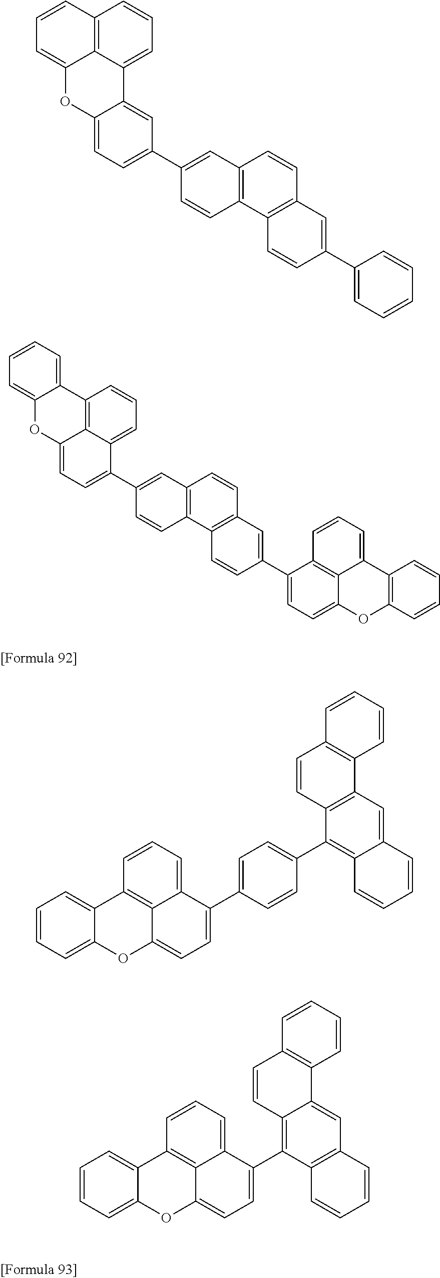 [SMILES: c1ccc(-c2ccc3c(ccc4cc(-c5ccc6c(c5)-c5cccc7cccc(c57)O6)ccc43)c2)cc1 ; c1ccc2c(c1)Oc1ccc(-c3ccc4c(ccc5cc(-c6ccc7c8c(cccc68)-c6ccccc6O7)ccc54)c3)c3cccc-2c13 ; c1ccc2c(c1)Oc1ccc(-c3ccc(-c4c5ccccc5cc5c4ccc4ccccc45)cc3)c3cccc-2c13 ; c1ccc2c(c1)Oc1ccc(-c3c4ccccc4cc4c3ccc3ccccc34)c3cccc-2c13]

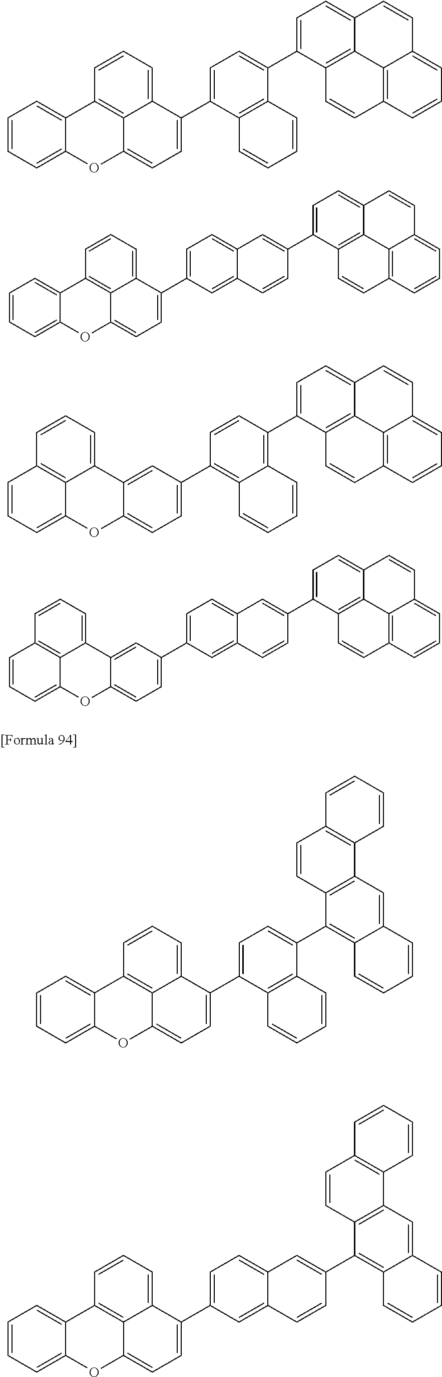 [SMILES: c1ccc2c(c1)Oc1ccc(-c3ccc(-c4ccc5ccc6cccc7ccc4c5c67)c4ccccc34)c3cccc-2c13 ; c1ccc2c(c1)Oc1ccc(-c3ccc4cc(-c5ccc6ccc7cccc8ccc5c6c78)ccc4c3)c3cccc-2c13 ; c1cc2c3c(cccc3c1)-c1cc(-c3ccc(-c4ccc5ccc6cccc7ccc4c5c67)c4ccccc34)ccc1O2 ; c1cc2c3c(cccc3c1)-c1cc(-c3ccc4cc(-c5ccc6ccc7cccc8ccc5c6c78)ccc4c3)ccc1O2 ; c1ccc2c(c1)Oc1ccc(-c3ccc(-c4c5ccccc5cc5c4ccc4ccccc45)c4ccccc34)c3cccc-2c13 ; c1ccc2c(c1)Oc1ccc(-c3ccc4cc(-c5c6ccccc6cc6c5ccc5ccccc56)ccc4c3)c3cccc-2c13]

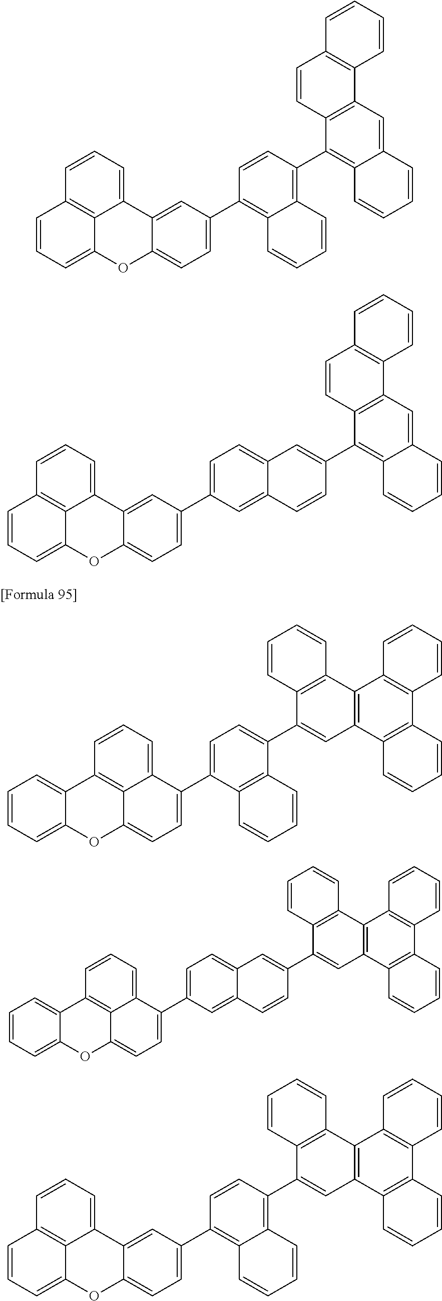

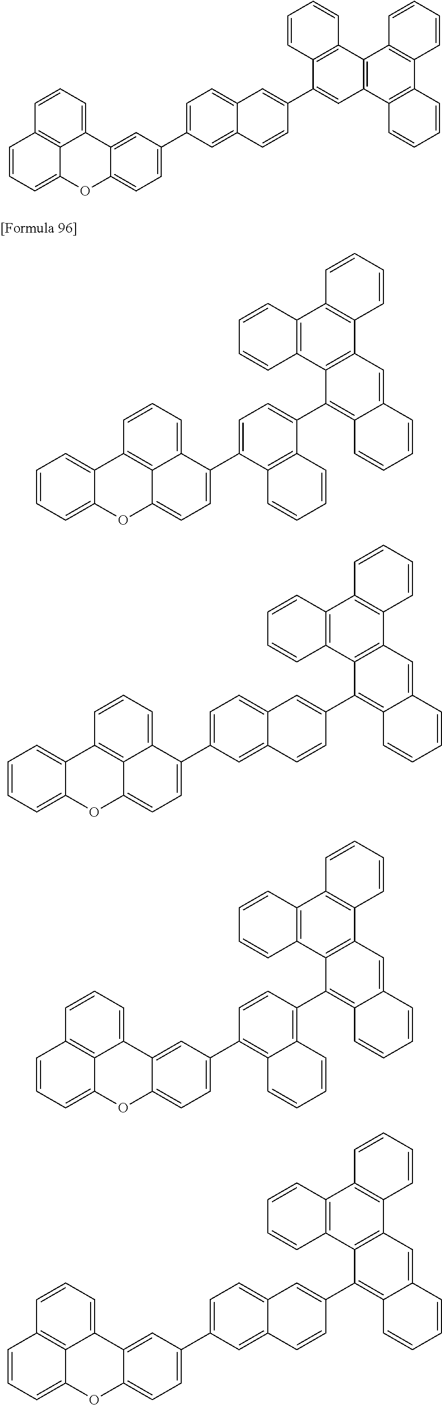 [SMILES: c1cc2c3c(cccc3c1)-c1cc(-c3ccc4cc(-c5cc6c7ccccc7c7ccccc7c6c6ccccc56)ccc4c3)ccc1O2 ; c1ccc2c(c1)Oc1ccc(-c3ccc(-c4c5ccccc5cc5c6ccccc6c6ccccc6c45)c4ccccc34)c3cccc-2c13 ; c1ccc2c(c1)Oc1ccc(-c3ccc4cc(-c5c6ccccc6cc6c7ccccc7c7ccccc7c56)ccc4c3)c3cccc-2c13 ; c1ccc2c(-c3ccc(-c4ccc5c(c4)-c4cccc6cccc(c46)O5)c4ccccc34)c3c4ccccc4c4ccccc4c3cc2c1 ; c1ccc2c(-c3ccc4cc(-c5ccc6c(c5)-c5cccc7cccc(c57)O6)ccc4c3)c3c4ccccc4c4ccccc4c3cc2c1]

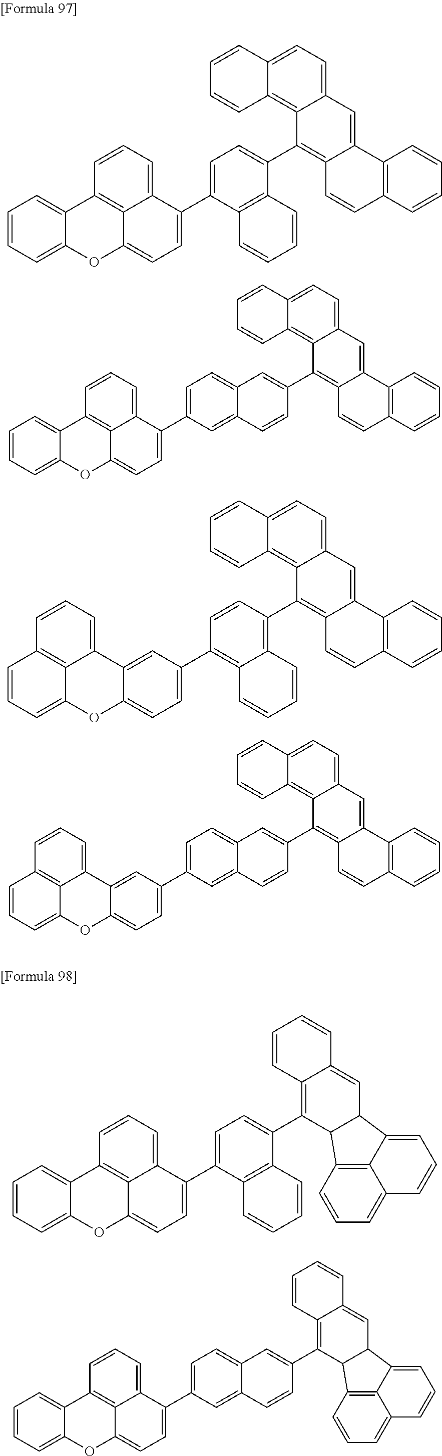

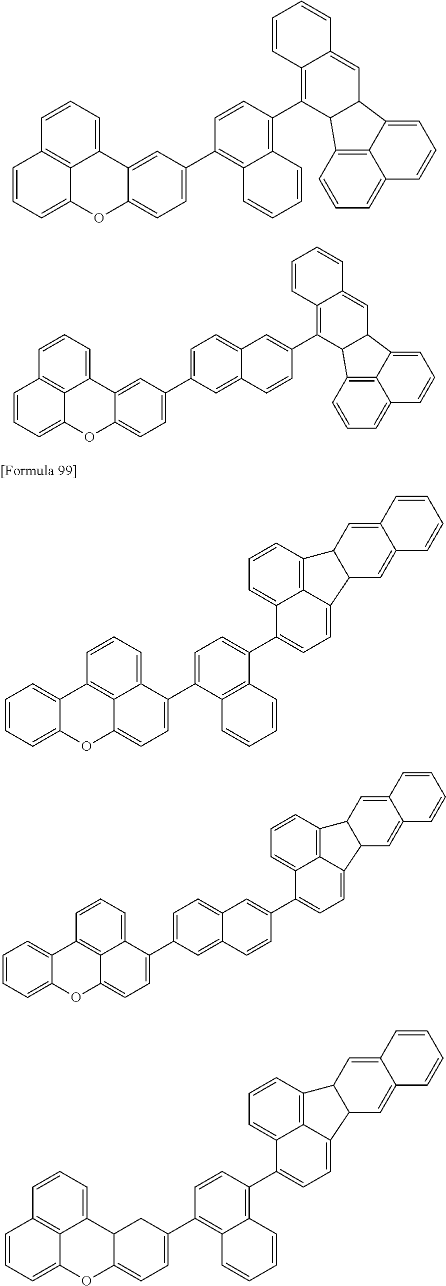

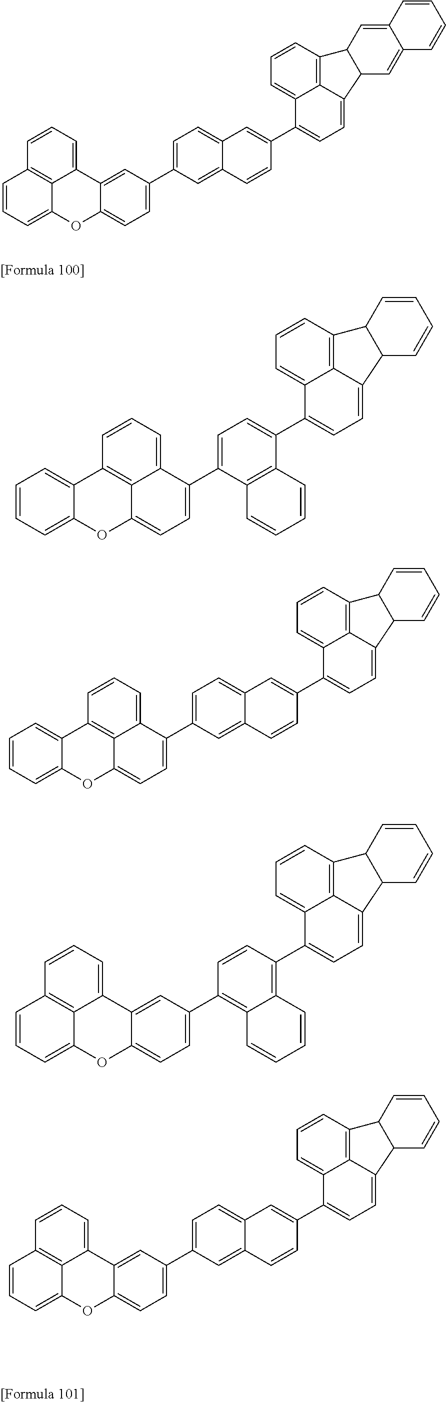 [SMILES: C1=c2ccccc2=CC2c3ccc(-c4ccc5cc(-c6ccc7c(c6)-c6cccc8cccc(c68)O7)ccc5c4)c4cccc(c34)C12 ; C1=CC2c3cccc4c(-c5ccc(-c6ccc7c8c(cccc68)-c6ccccc6O7)c6ccccc56)ccc(c34)C2C=C1 ; C1=CC2c3cccc4c(-c5ccc6cc(-c7ccc8c9c(cccc79)-c7ccccc7O8)ccc6c5)ccc(c34)C2C=C1 ; C1=CC2c3cccc4c(-c5ccc(-c6ccc7c(c6)-c6cccc8cccc(c68)O7)c6ccccc56)ccc(c34)C2C=C1 ; C1=CC2c3cccc4c(-c5ccc6cc(-c7ccc8c(c7)-c7cccc9cccc(c79)O8)ccc6c5)ccc(c34)C2C=C1]

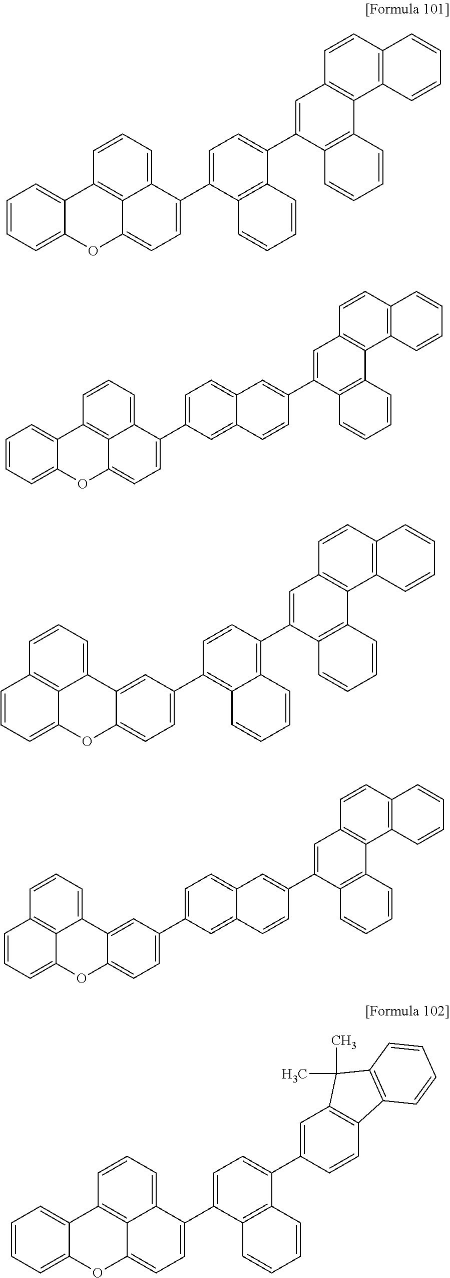 [SMILES: c1ccc2c(c1)Oc1ccc(-c3ccc(-c4cc5ccc6ccccc6c5c5ccccc45)c4ccccc34)c3cccc-2c13 ; c1ccc2c(c1)Oc1ccc(-c3ccc4cc(-c5cc6ccc7ccccc7c6c6ccccc56)ccc4c3)c3cccc-2c13 ; c1cc2c3c(cccc3c1)-c1cc(-c3ccc(-c4cc5ccc6ccccc6c5c5ccccc45)c4ccccc34)ccc1O2 ; c1cc2c3c(cccc3c1)-c1cc(-c3ccc4cc(-c5cc6ccc7ccccc7c6c6ccccc56)ccc4c3)ccc1O2 ; CC1(C)c2ccccc2-c2ccc(-c3ccc(-c4ccc5c6c(cccc46)-c4ccccc4O5)c4ccccc34)cc21]

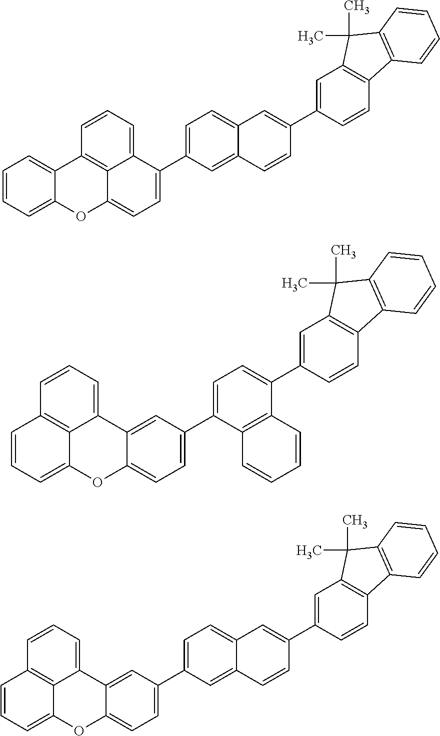 [SMILES: CC1(C)c2ccccc2-c2ccc(-c3ccc4cc(-c5ccc6c7c(cccc57)-c5ccccc5O6)ccc4c3)cc21 ; CC1(C)c2ccccc2-c2ccc(-c3ccc(-c4ccc5c(c4)-c4cccc6cccc(c46)O5)c4ccccc34)cc21 ; CC1(C)c2ccccc2-c2ccc(-c3ccc4cc(-c5ccc6c(c5)-c5cccc7cccc(c57)O6)ccc4c3)cc21]

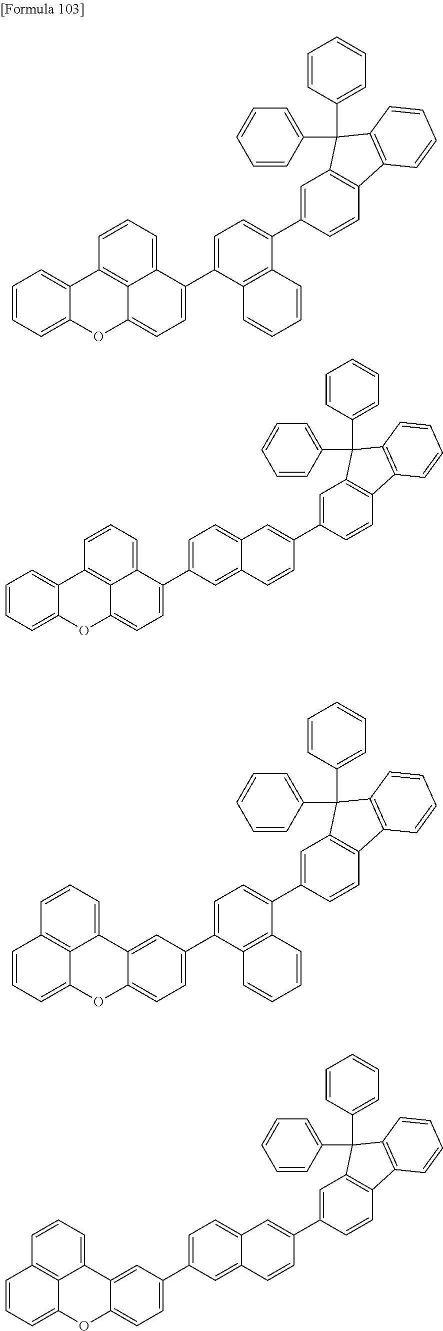 [SMILES: c1ccc(C2(c3ccccc3)c3ccccc3-c3ccc(-c4ccc(-c5ccc6c7c(cccc57)-c5ccccc5O6)c5ccccc45)cc32)cc1 ; c1ccc(C2(c3ccccc3)c3ccccc3-c3ccc(-c4ccc5cc(-c6ccc7c8c(cccc68)-c6ccccc6O7)ccc5c4)cc32)cc1 ; c1ccc(C2(c3ccccc3)c3ccccc3-c3ccc(-c4ccc(-c5ccc6c(c5)-c5cccc7cccc(c57)O6)c5ccccc45)cc32)cc1 ; c1ccc(C2(c3ccccc3)c3ccccc3-c3ccc(-c4ccc5cc(-c6ccc7c(c6)-c6cccc8cccc(c68)O7)ccc5c4)cc32)cc1]

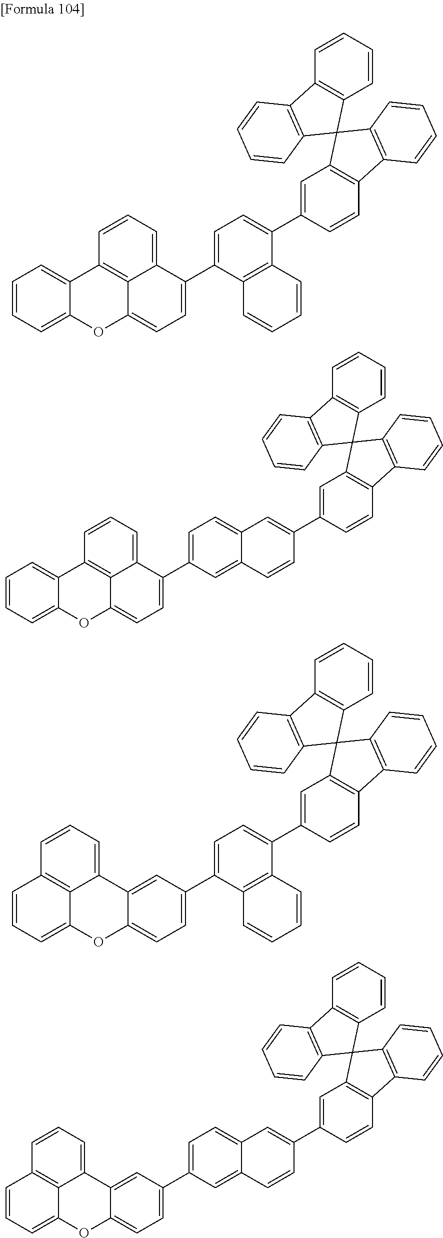 [SMILES: c1ccc2c(c1)Oc1ccc(-c3ccc(-c4ccc5c(c4)C4(c6ccccc6-c6ccccc64)c4ccccc4-5)c4ccccc34)c3cccc-2c13 ; c1ccc2c(c1)Oc1ccc(-c3ccc4cc(-c5ccc6c(c5)C5(c7ccccc7-c7ccccc75)c5ccccc5-6)ccc4c3)c3cccc-2c13 ; c1ccc2c(c1)-c1ccccc1C21c2ccccc2-c2ccc(-c3ccc(-c4ccc5c(c4)-c4cccc6cccc(c46)O5)c4ccccc34)cc21 ; c1ccc2c(c1)-c1ccccc1C21c2ccccc2-c2ccc(-c3ccc4cc(-c5ccc6c(c5)-c5cccc7cccc(c57)O6)ccc4c3)cc21]

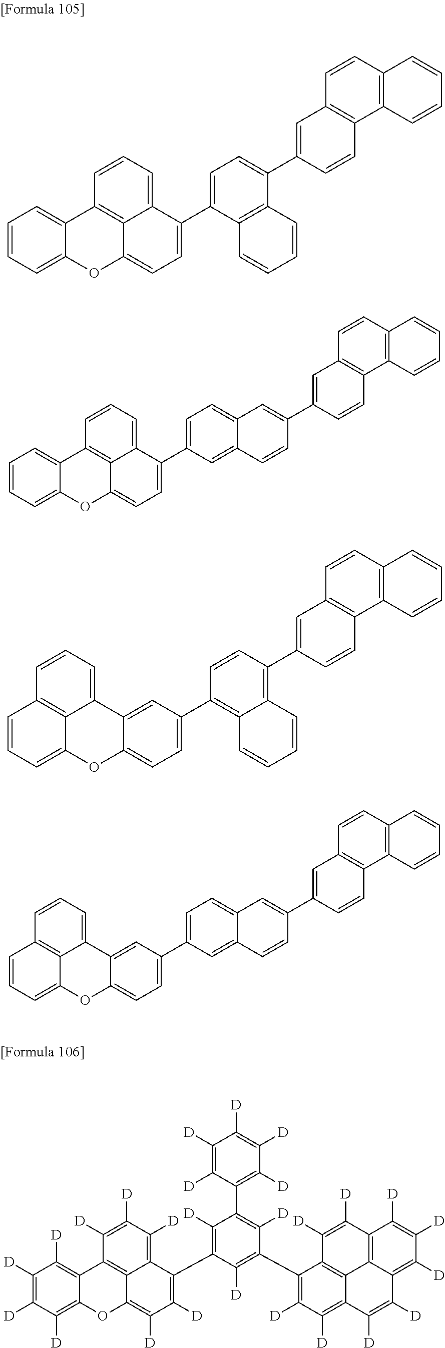

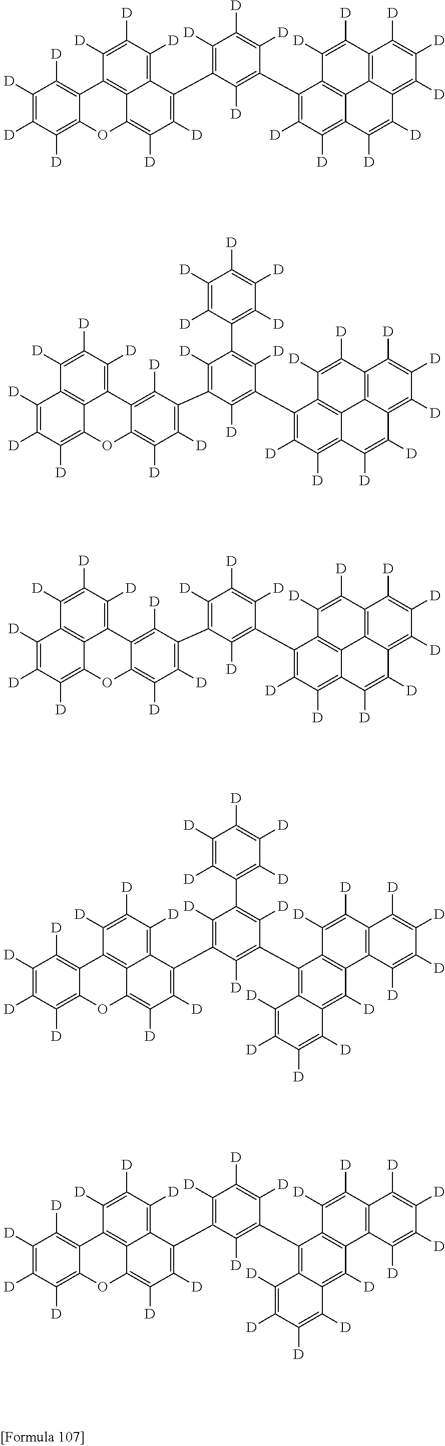

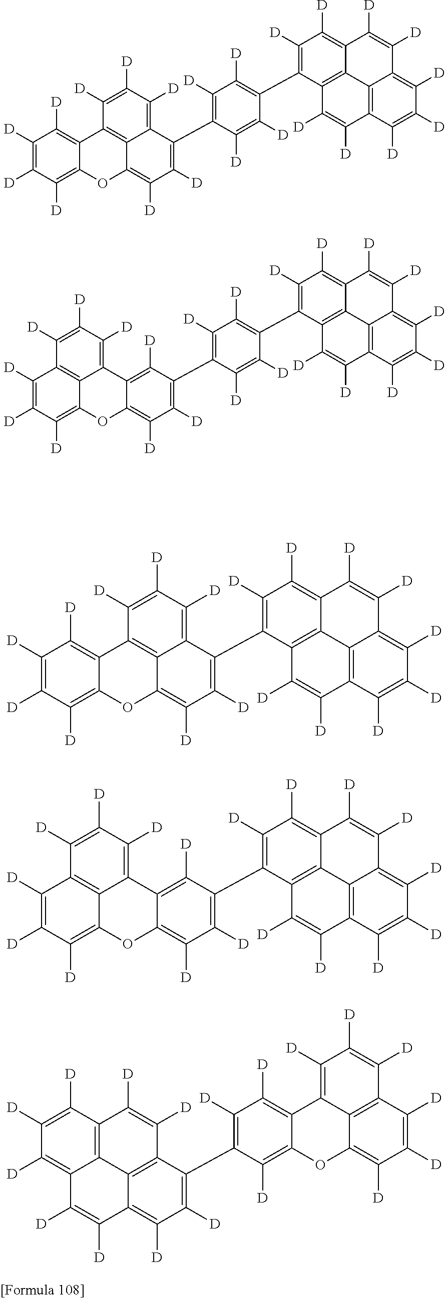

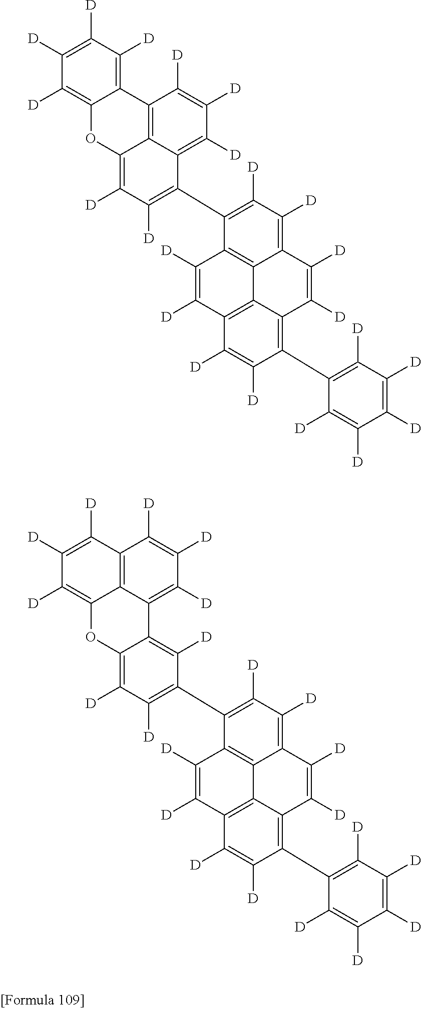 [SMILES: [2H]c1c([2H])c([2H])c(-c2c([2H])c([2H])c3c([2H])c([2H])c4c(-c5c([2H])c([2H])c6c7c(c([2H])c([2H])c([2H])c57)-c5c([2H])c([2H])c([2H])c([2H])c5O6)c([2H])c([2H])c5c([2H])c([2H])c2c3c54)c([2H])c1[2H] ; [2H]c1c([2H])c([2H])c(-c2c([2H])c([2H])c3c([2H])c([2H])c4c(-c5c([2H])c([2H])c6c(c5[2H])-c5c([2H])c([2H])c([2H])c7c([2H])c([2H])c([2H])c(c57)O6)c([2H])c([2H])c5c([2H])c([2H])c2c3c54)c([2H])c1[2H]]

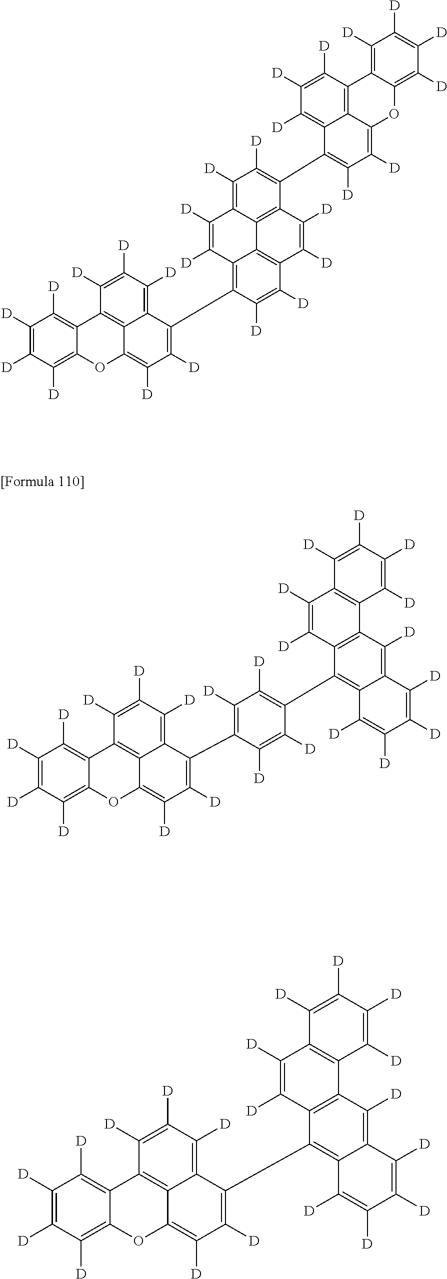

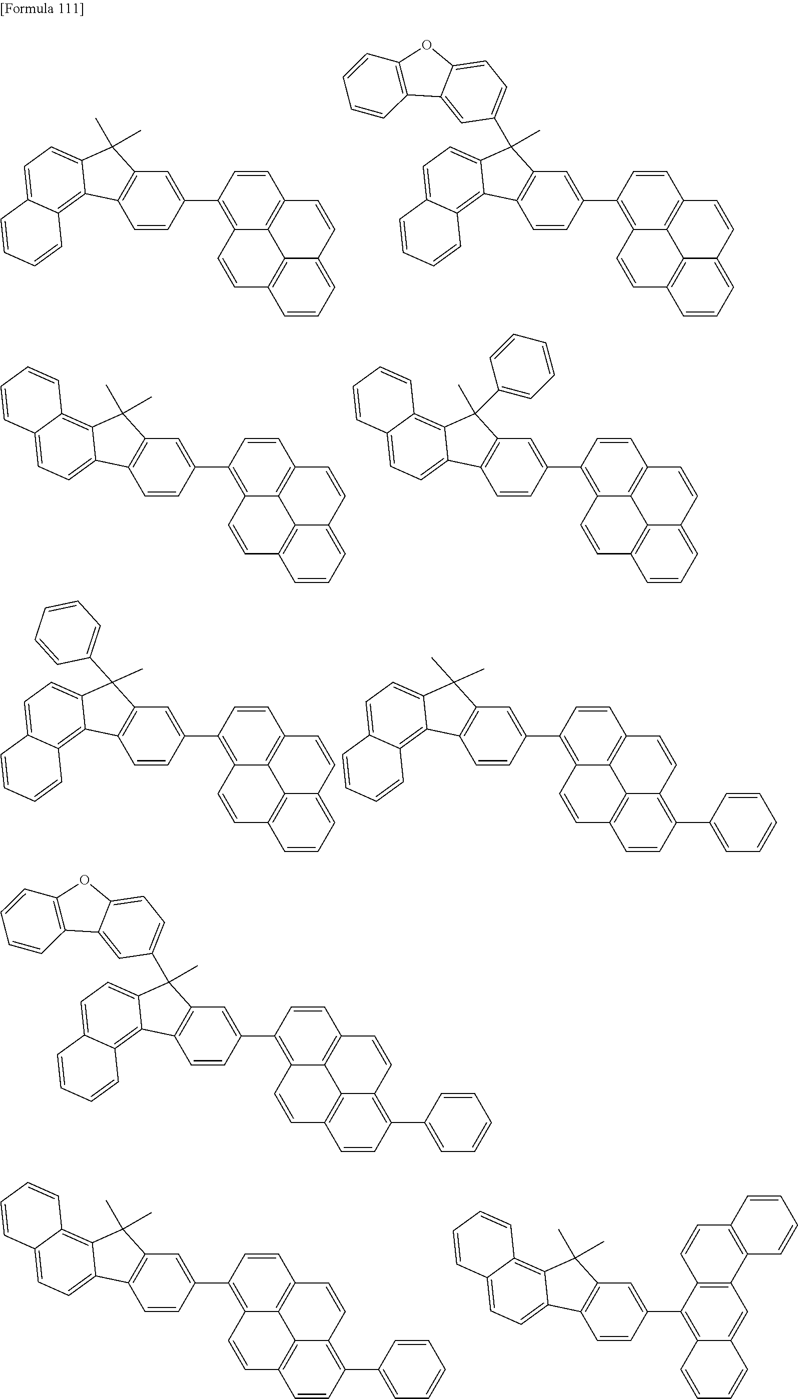

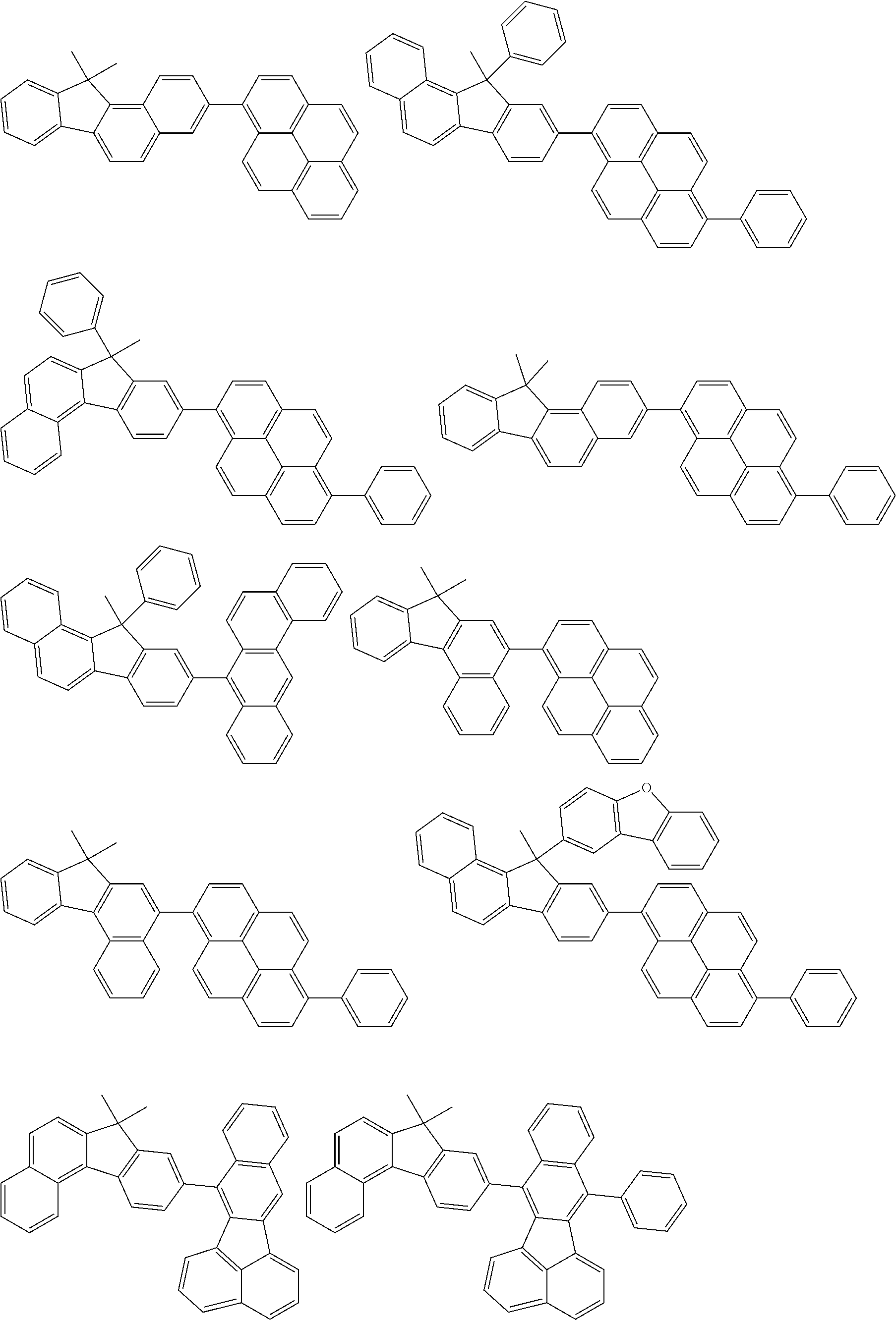

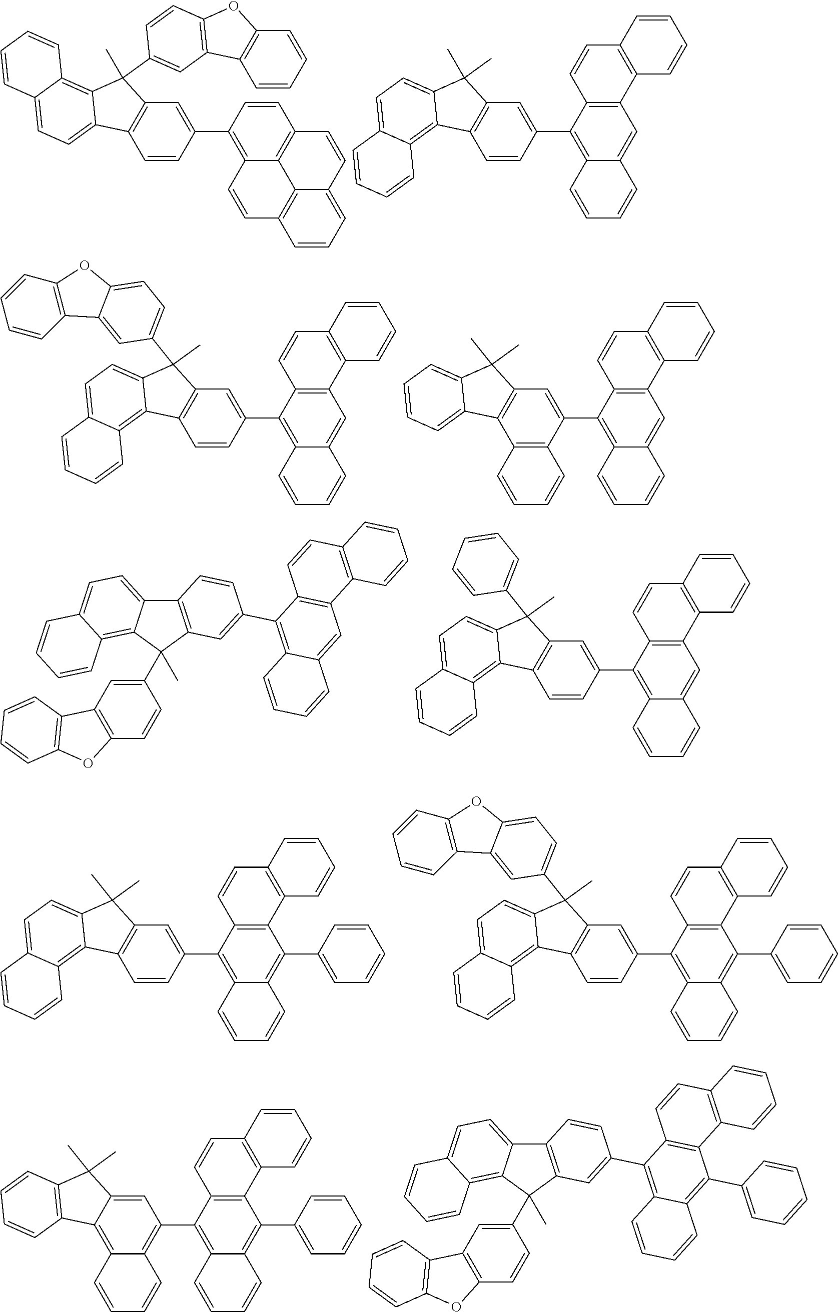

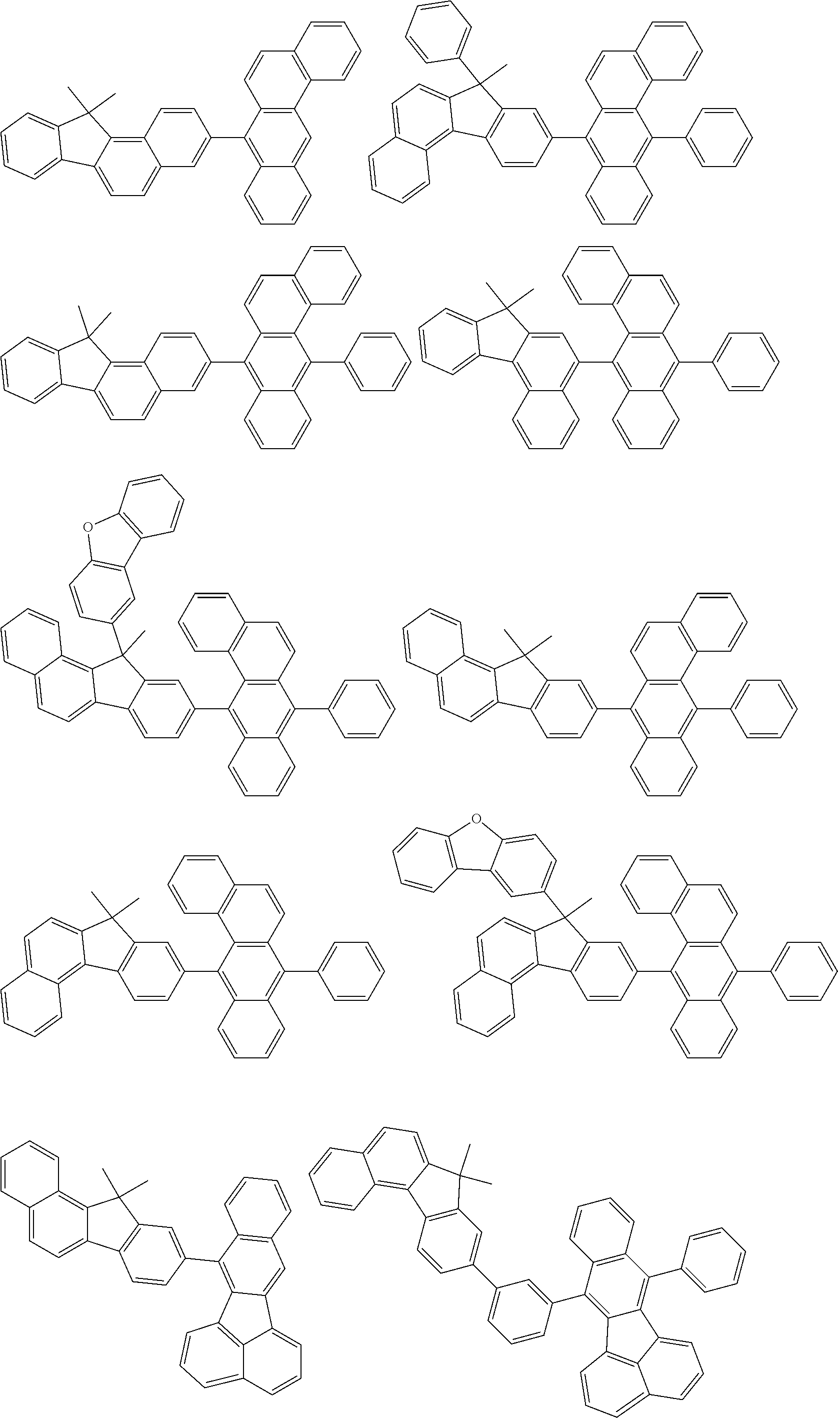

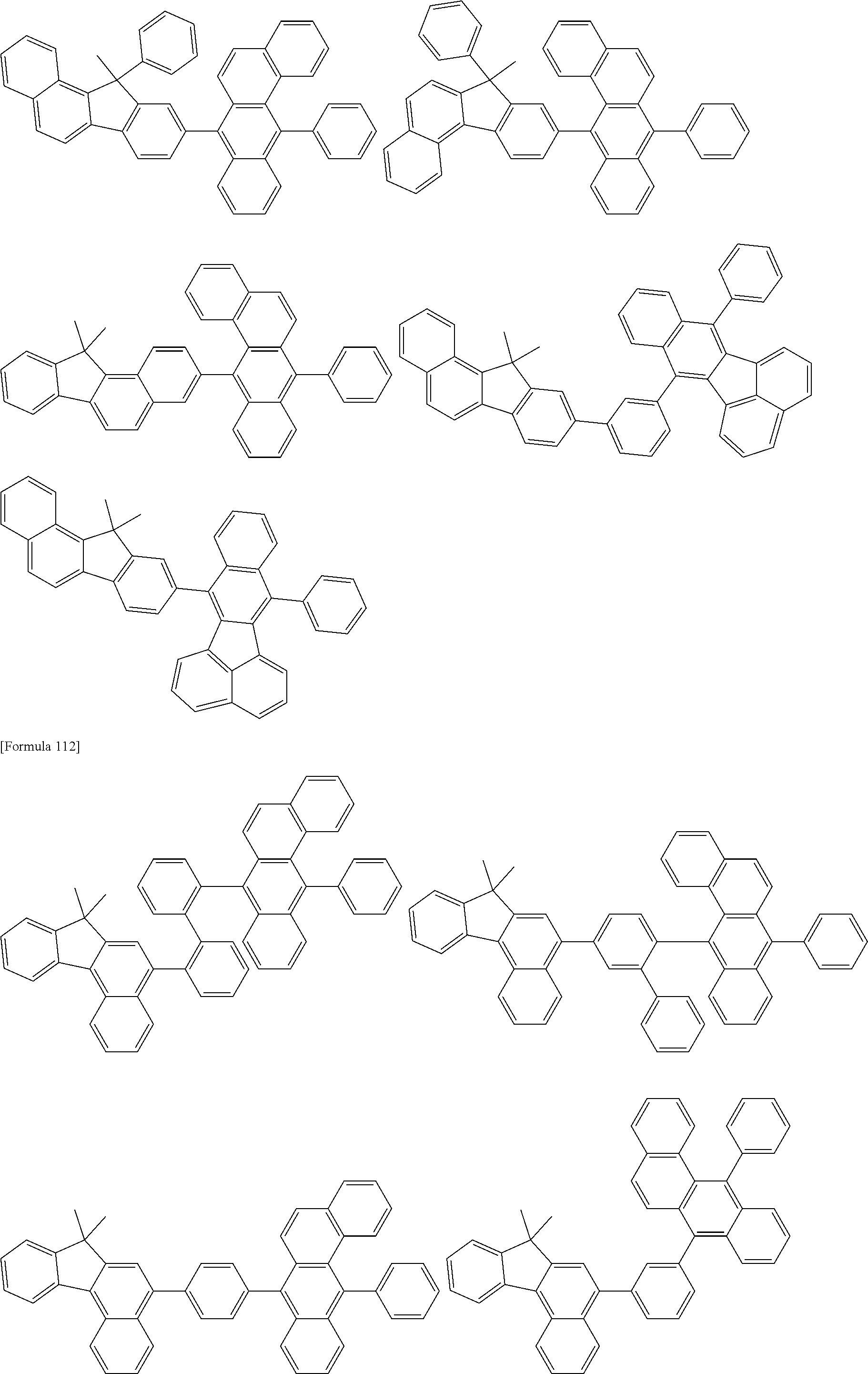

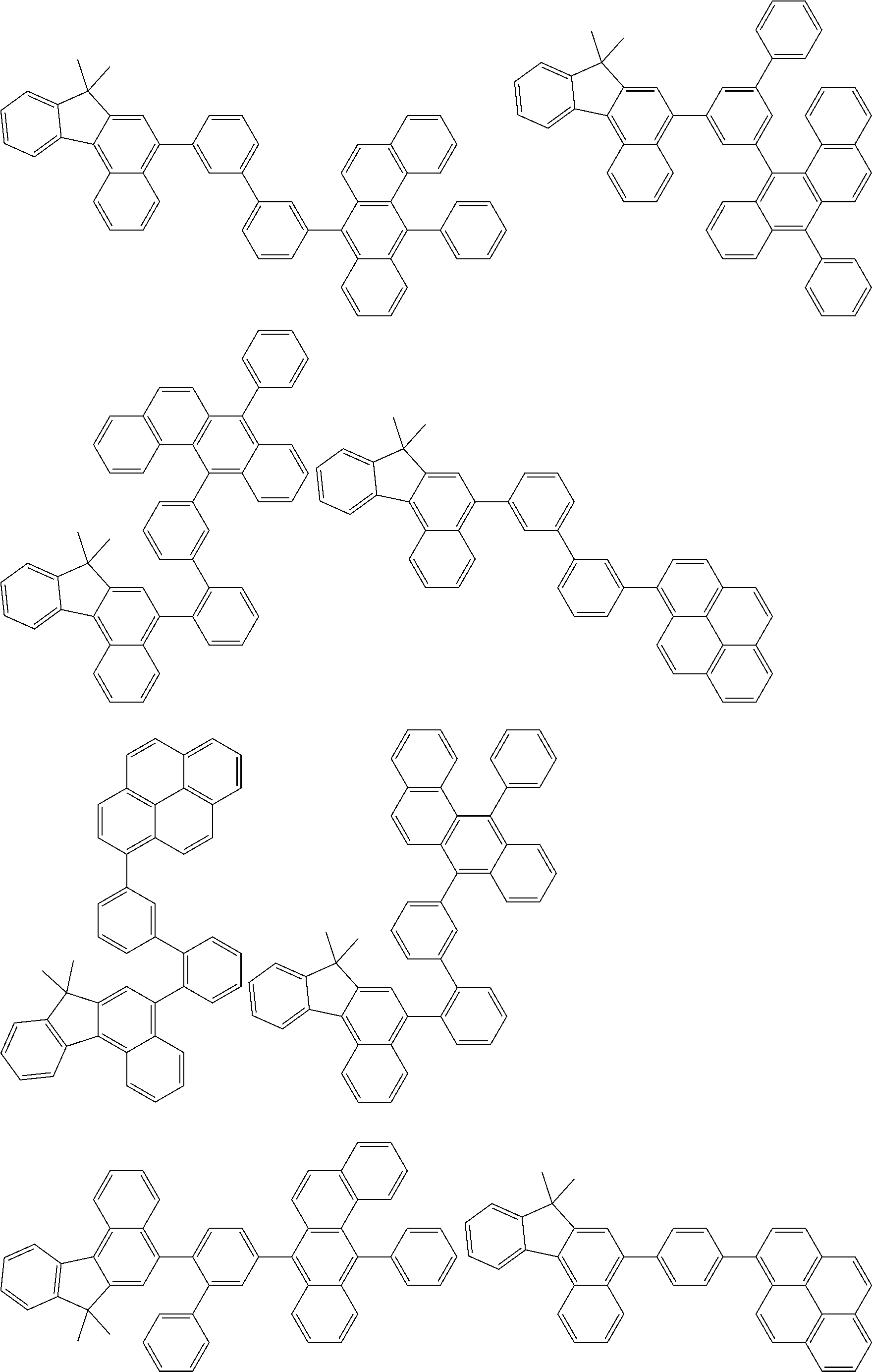

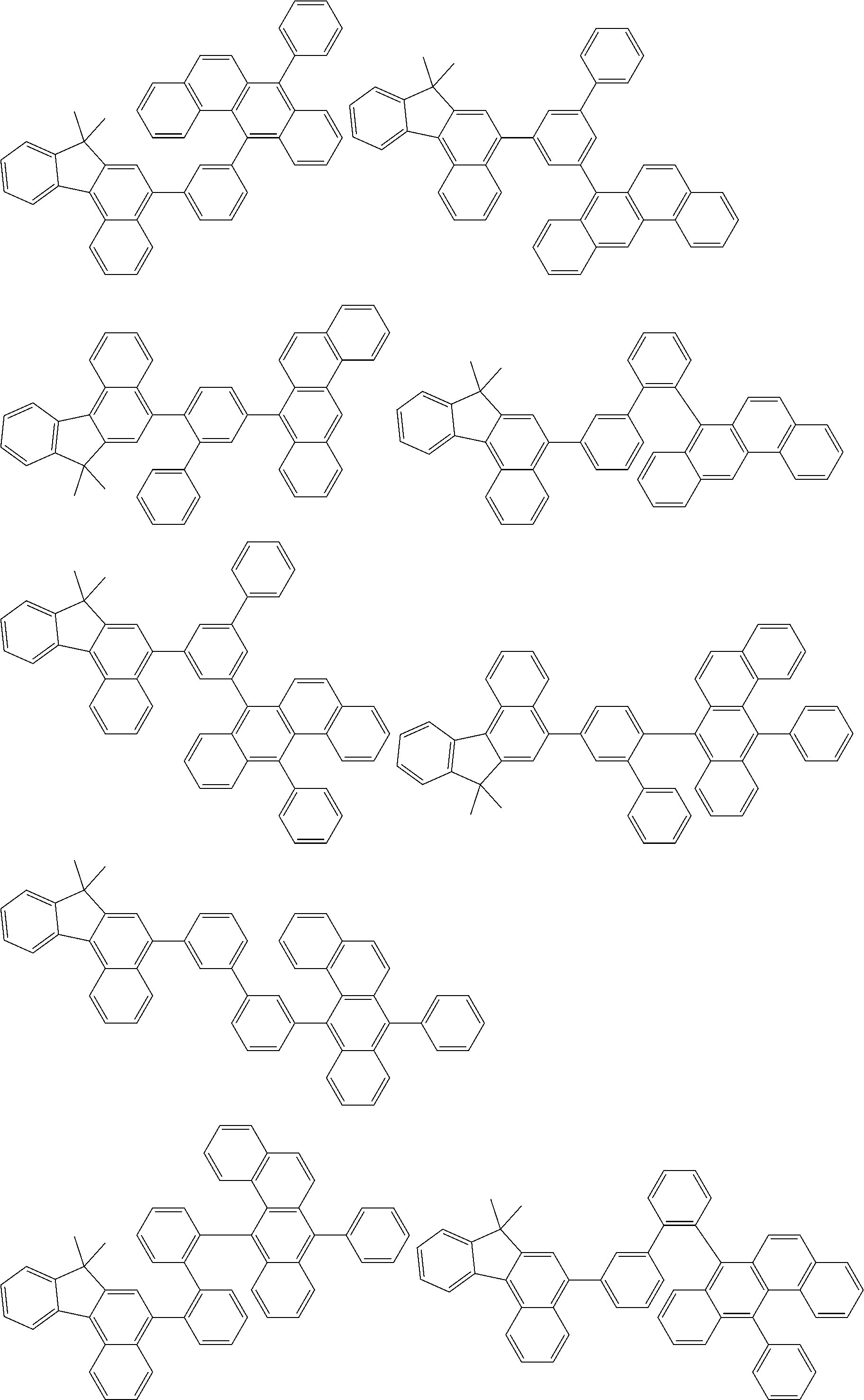

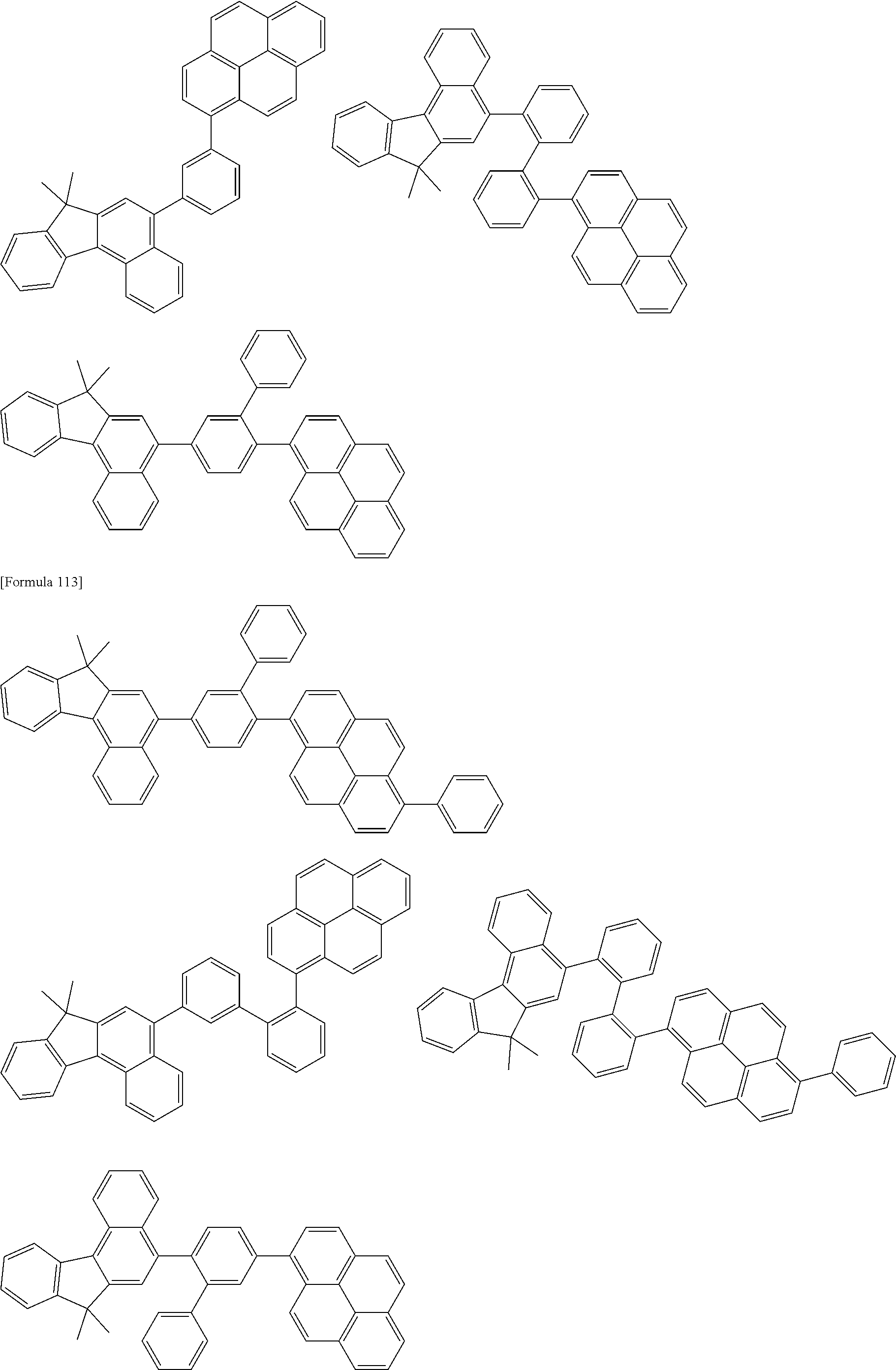

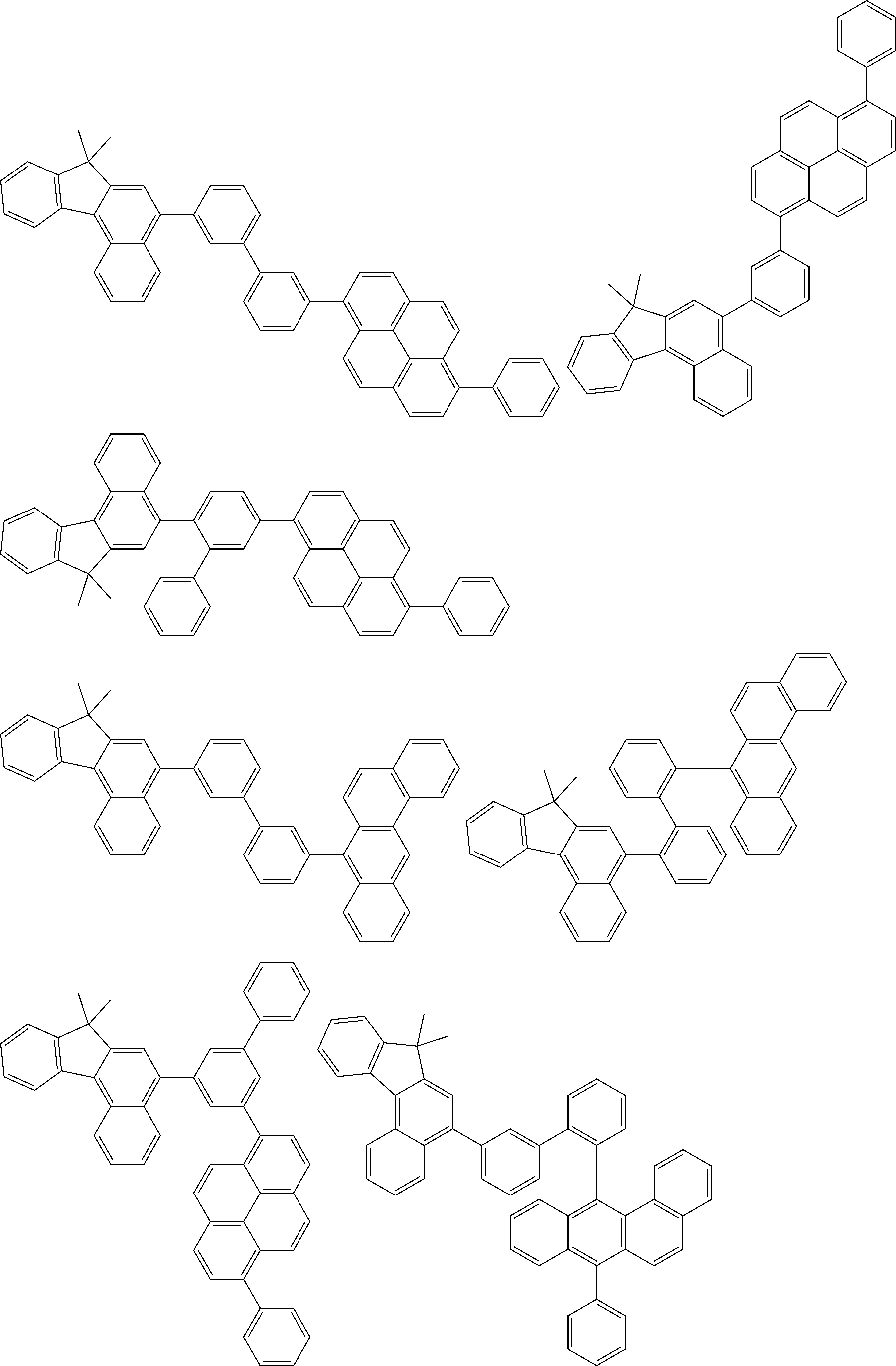

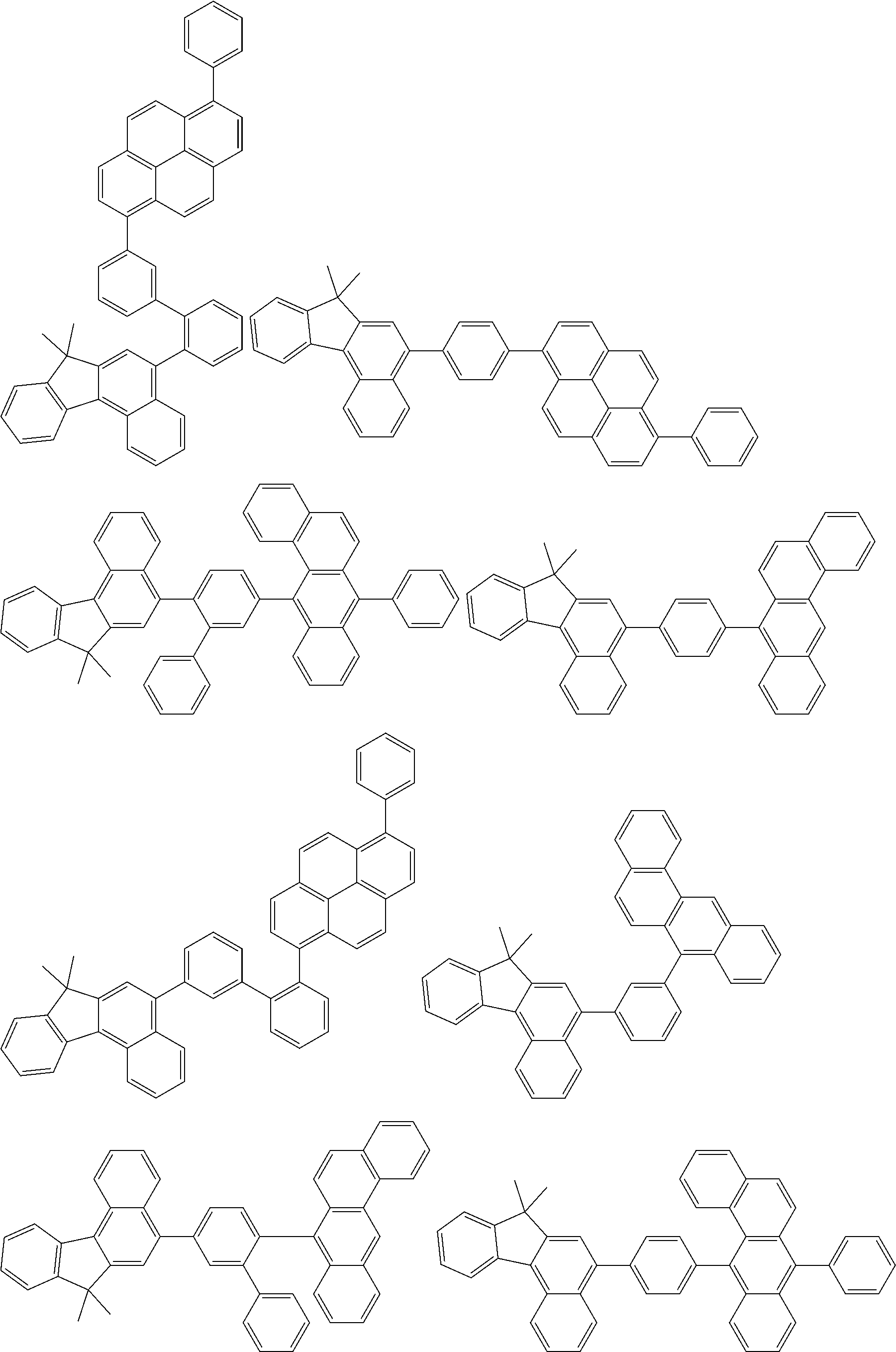

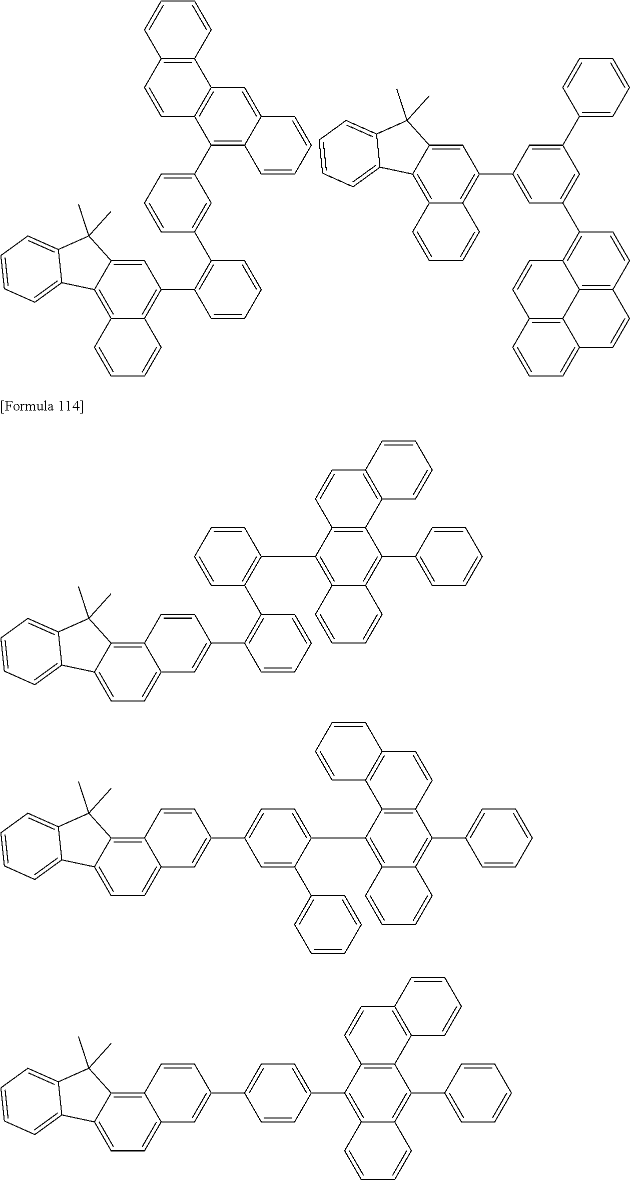

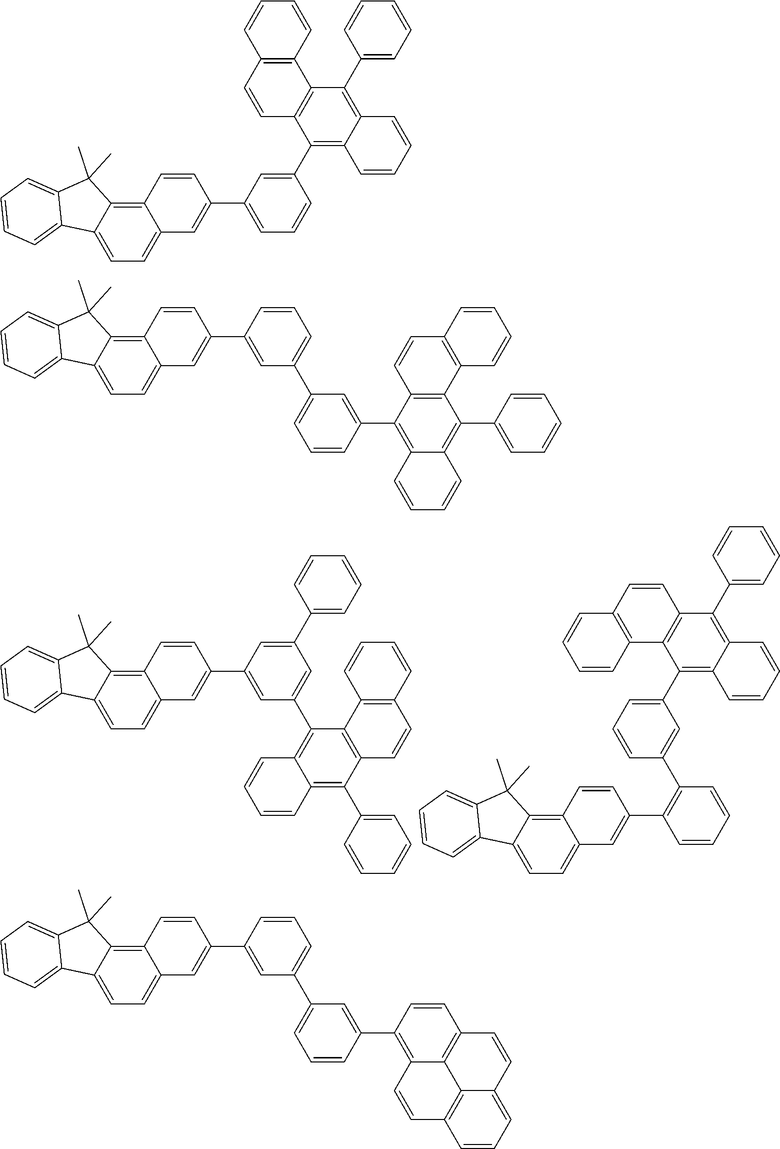

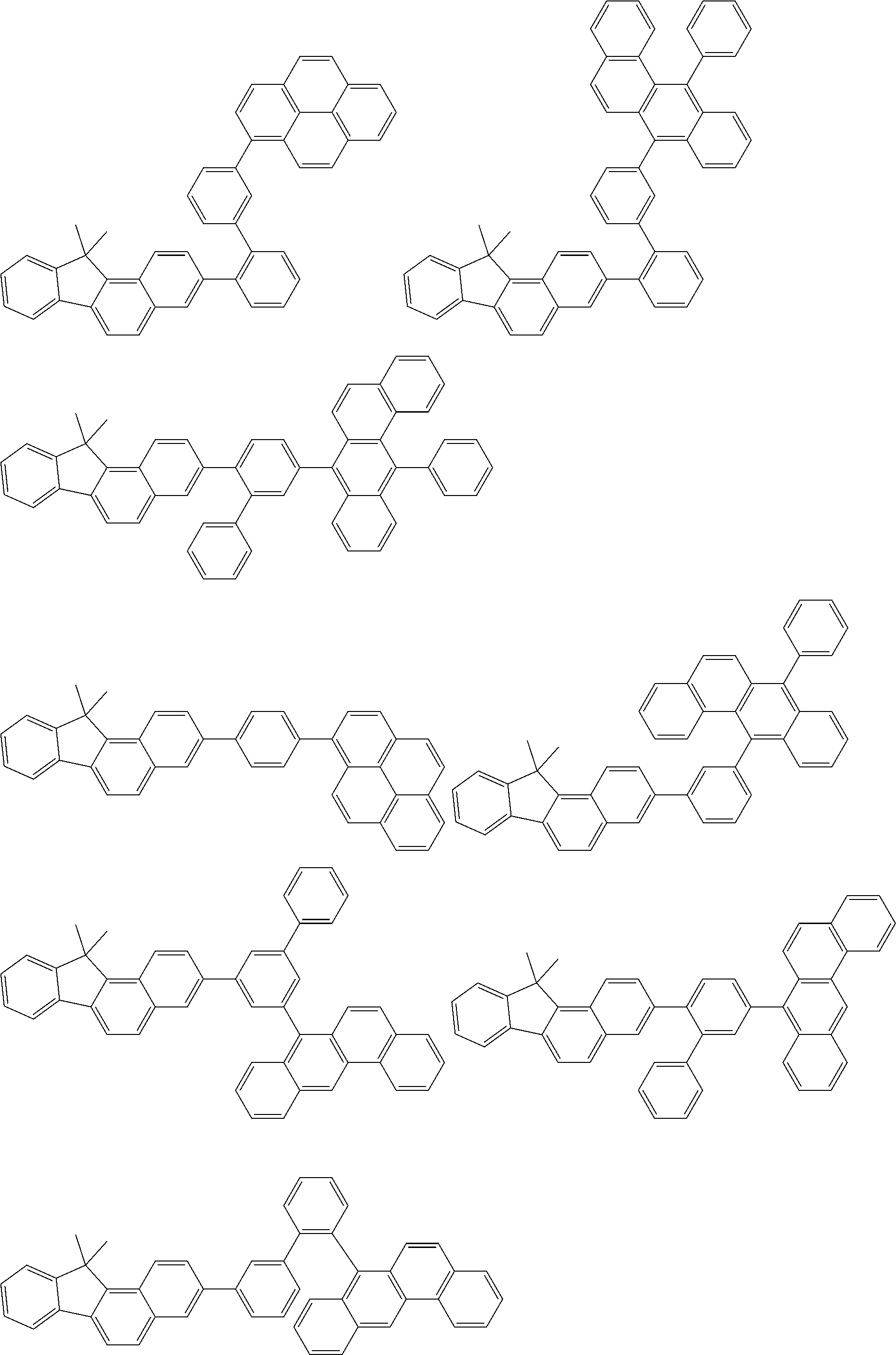

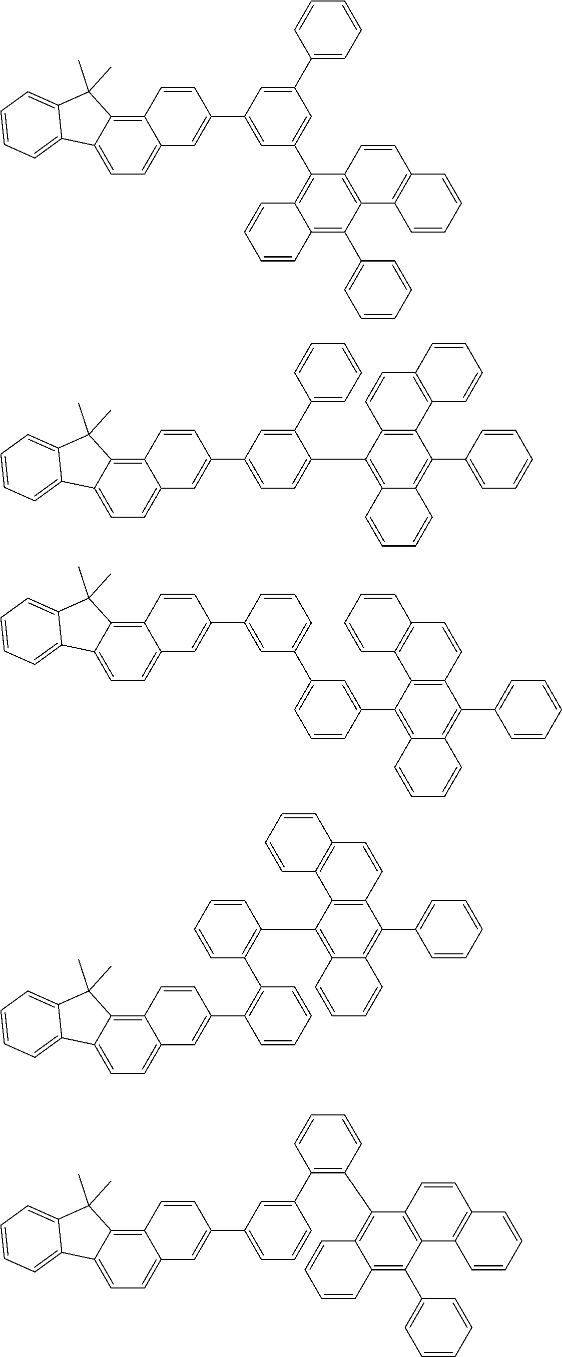

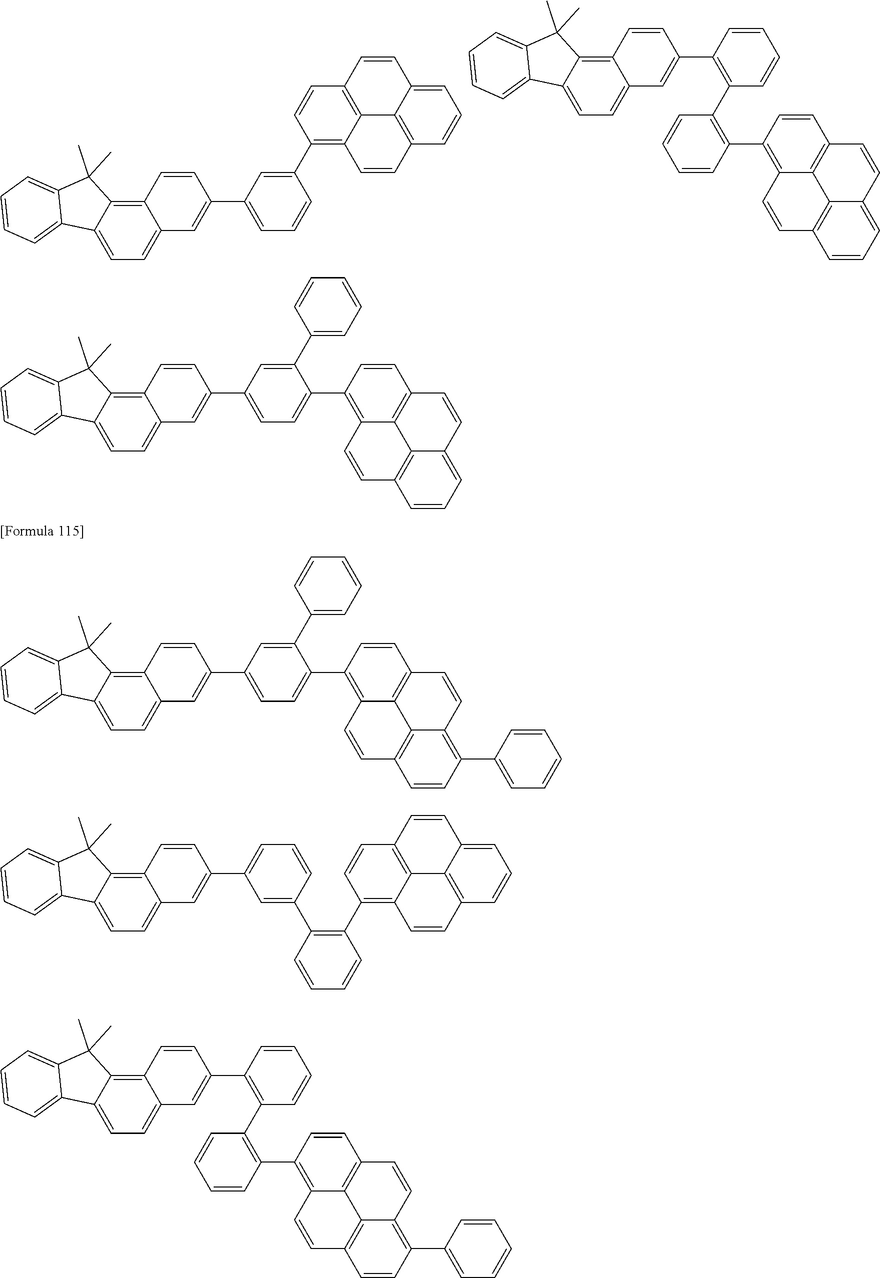

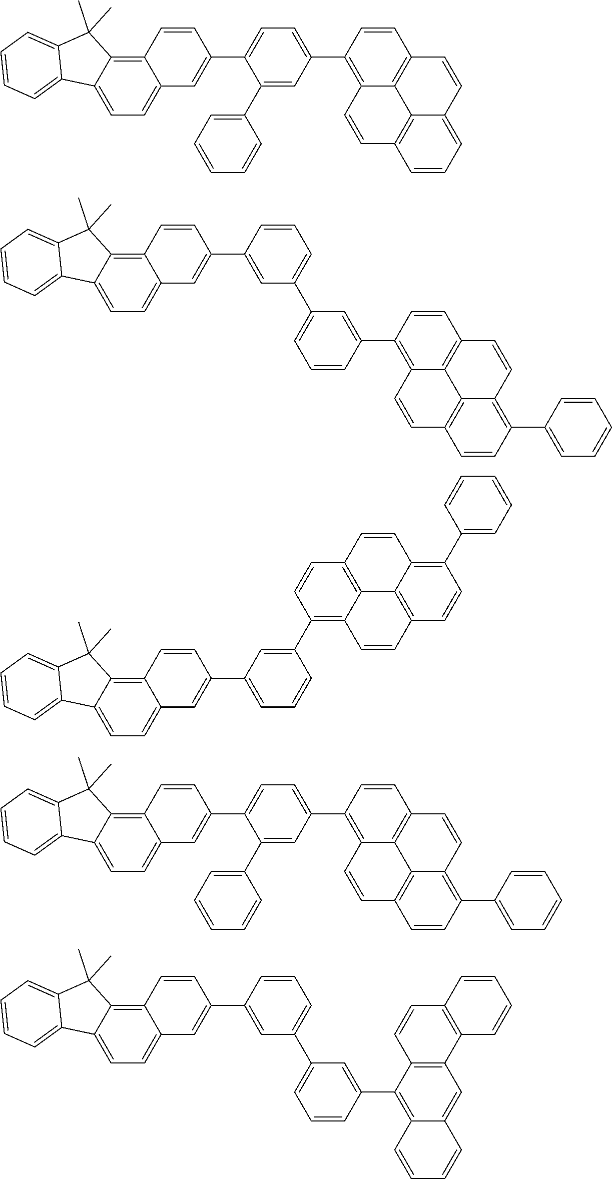

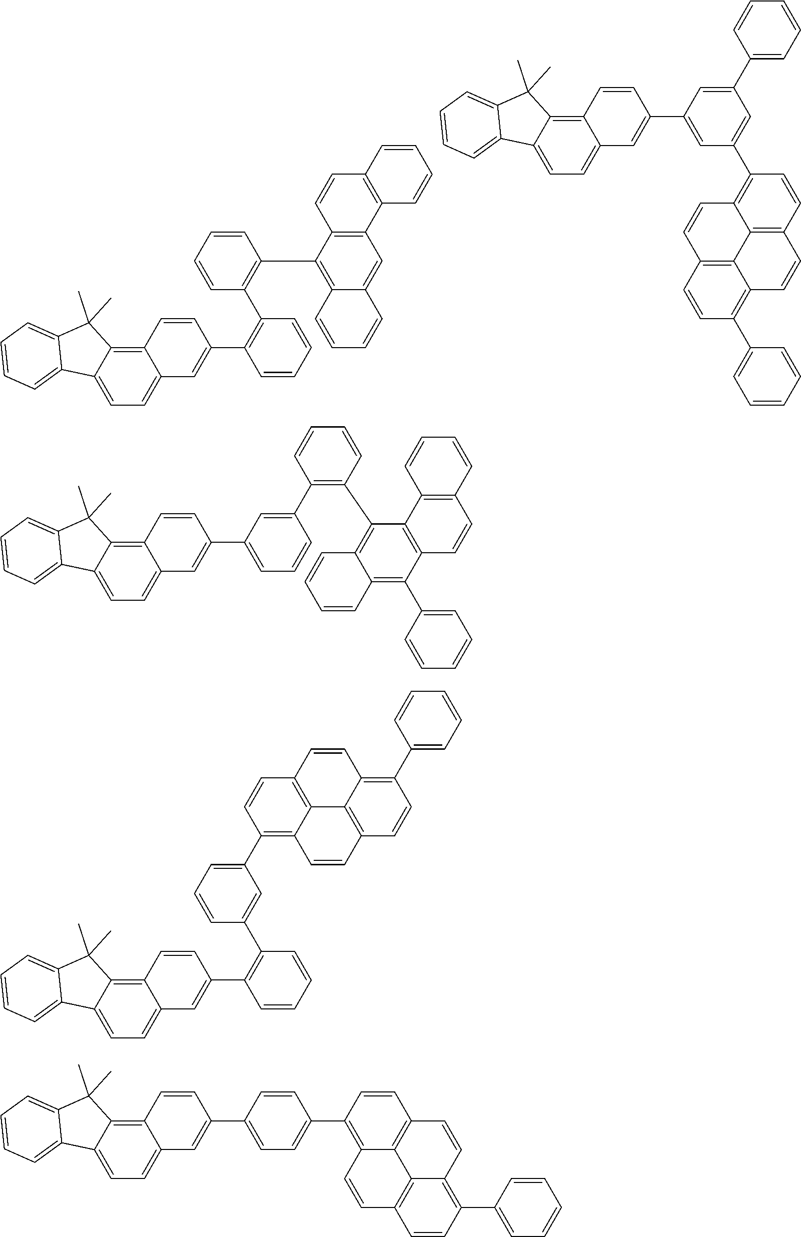

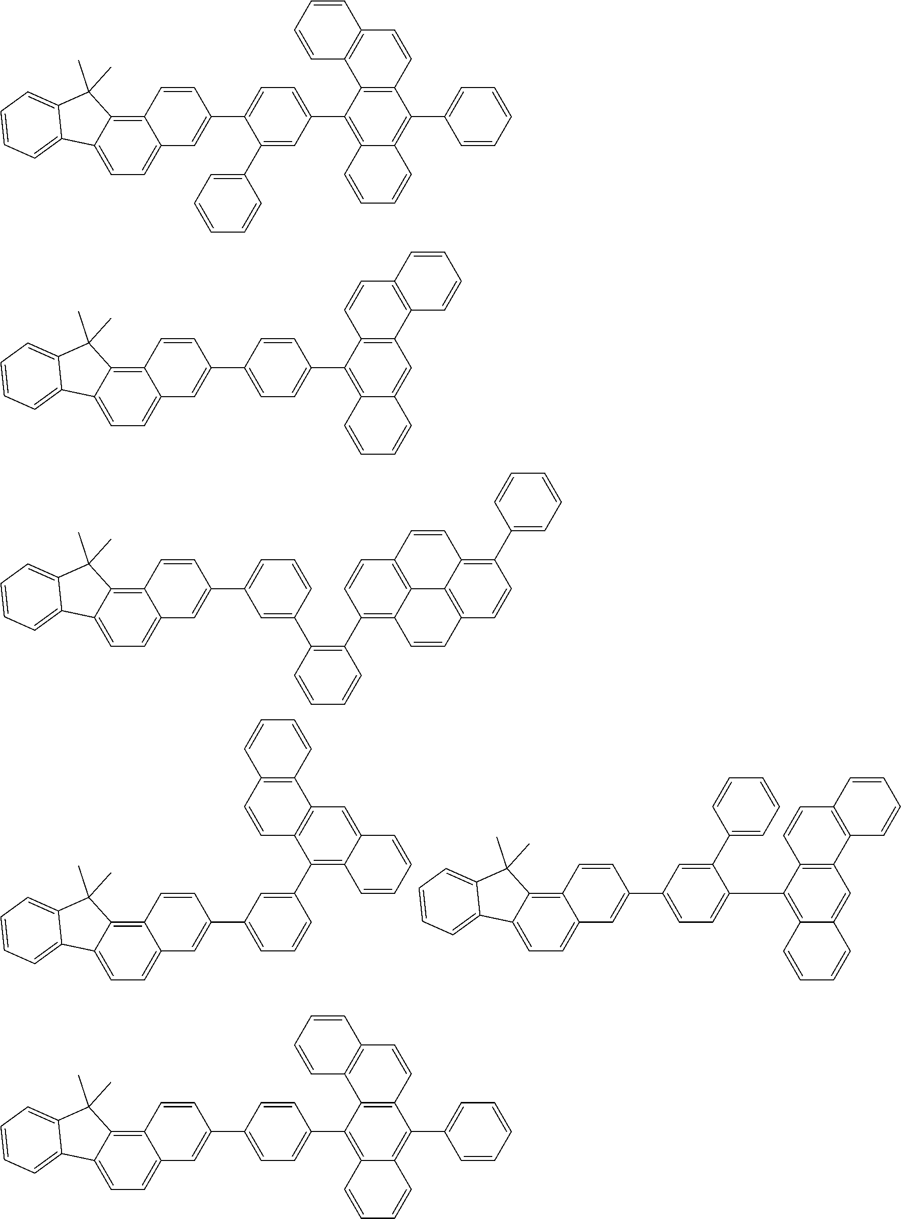

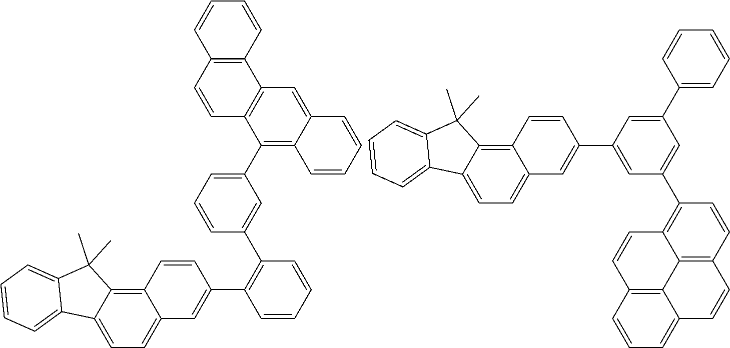

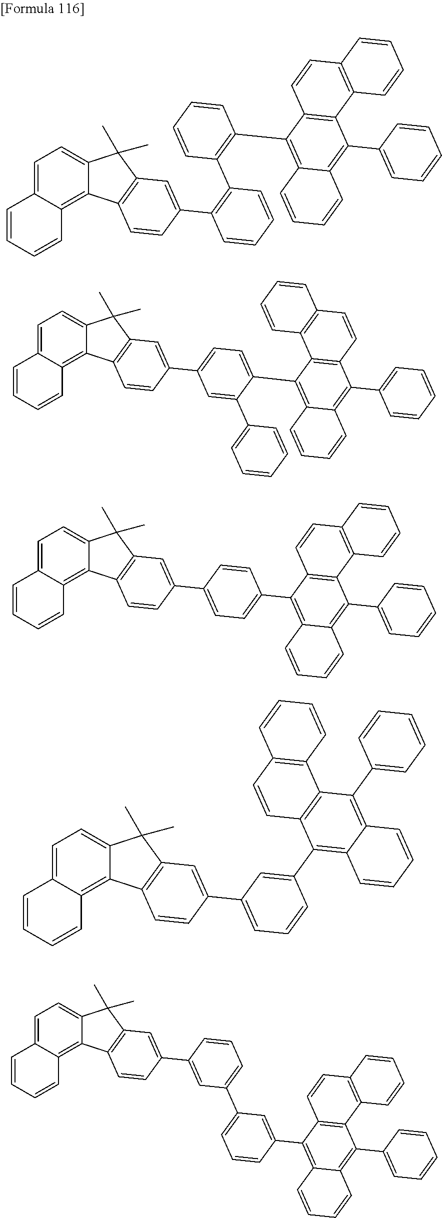

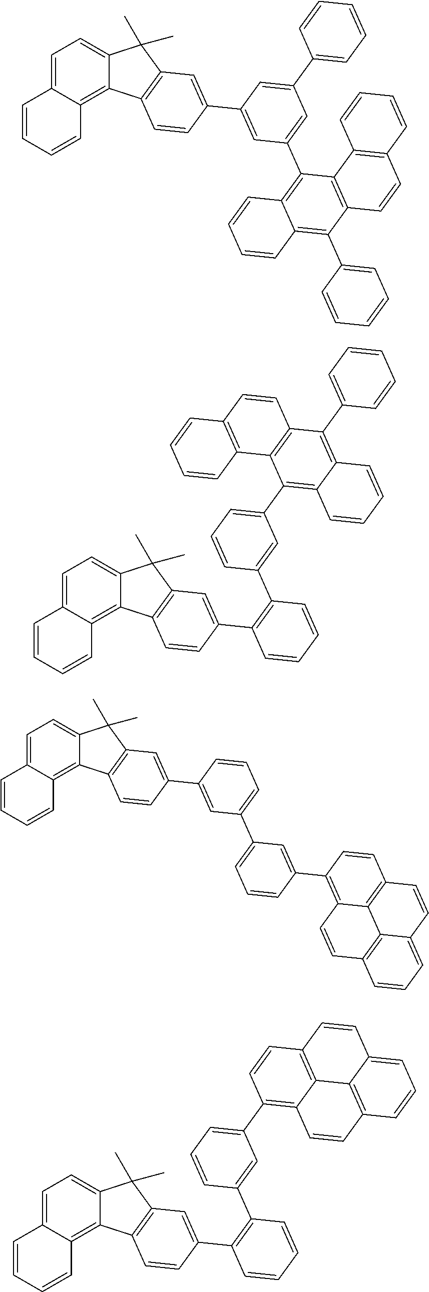

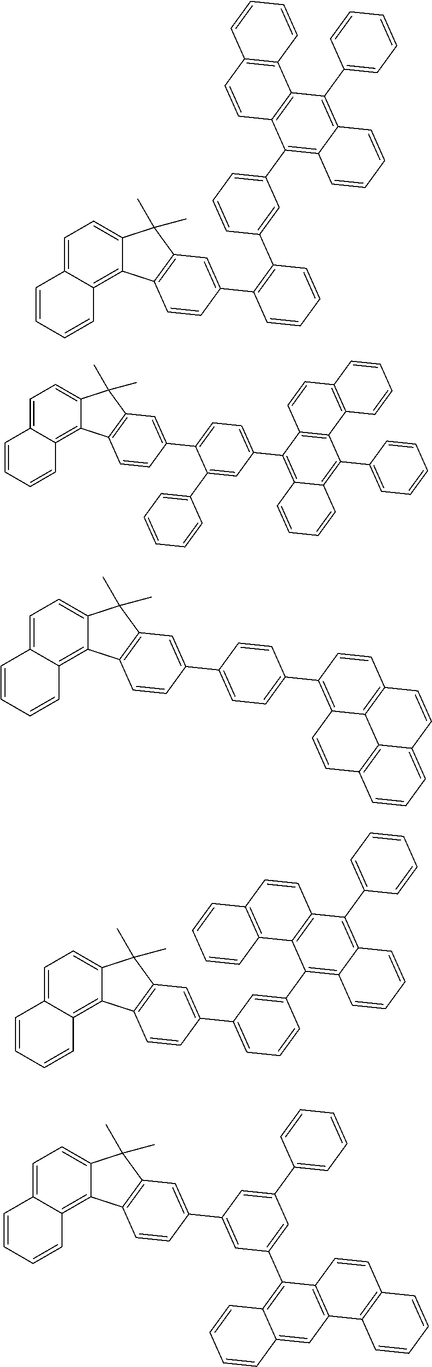

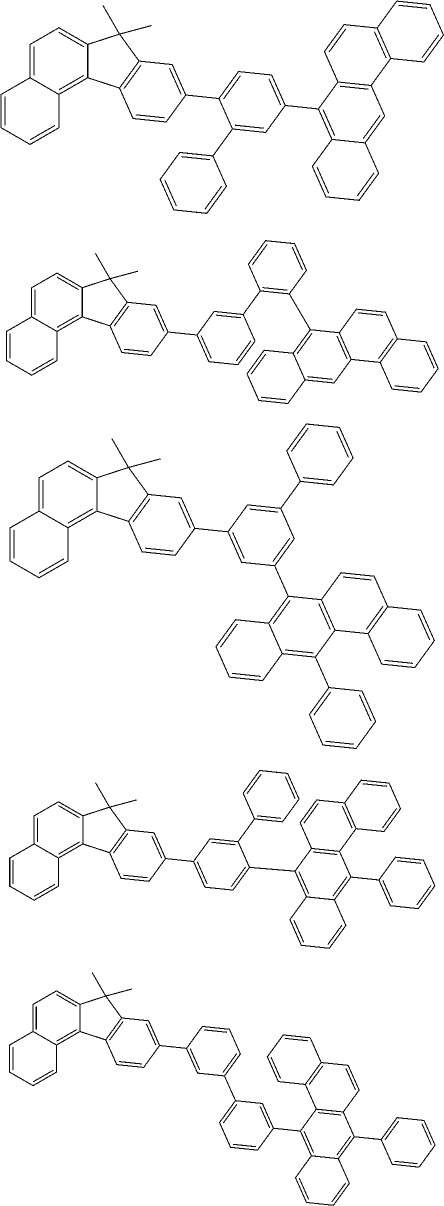

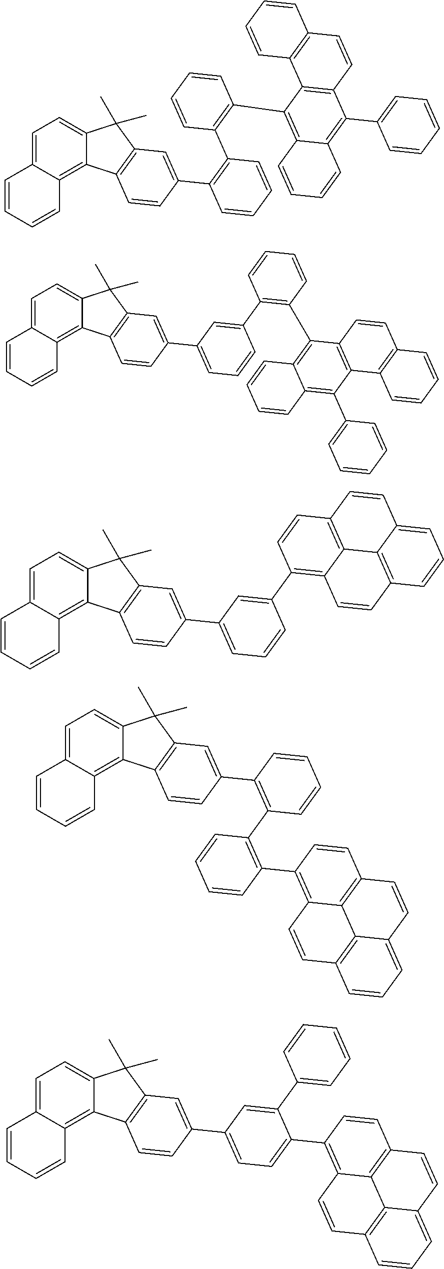

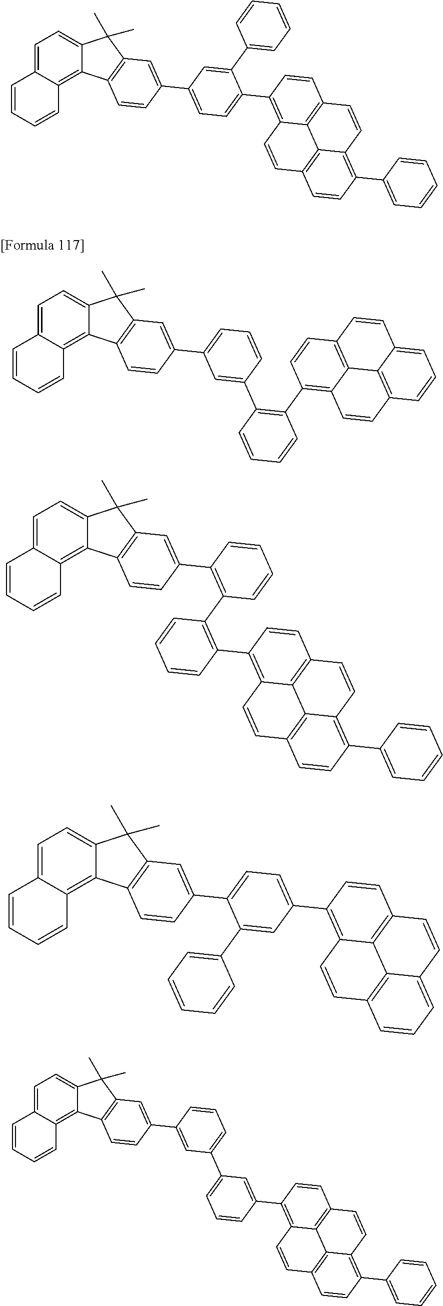

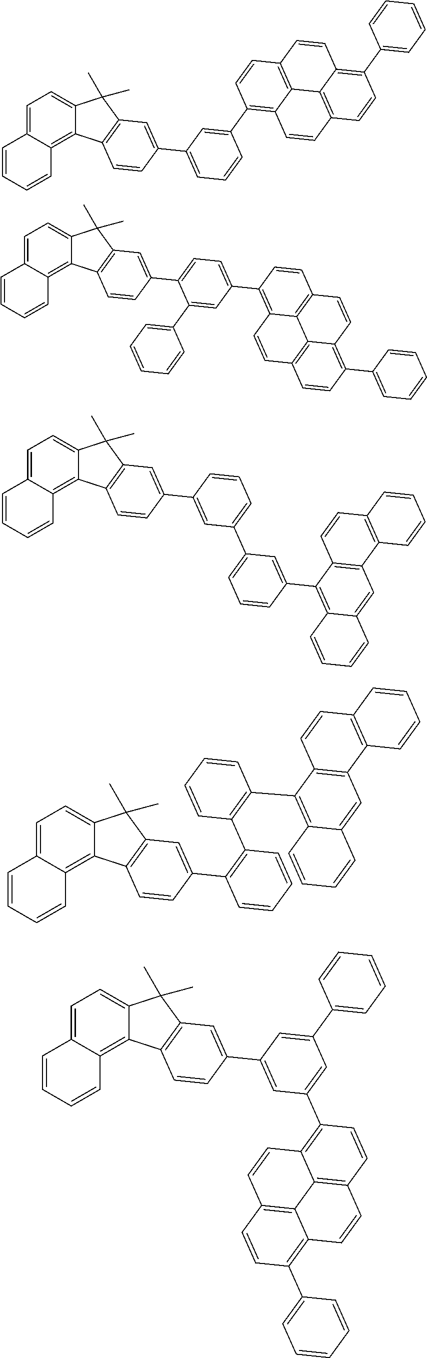

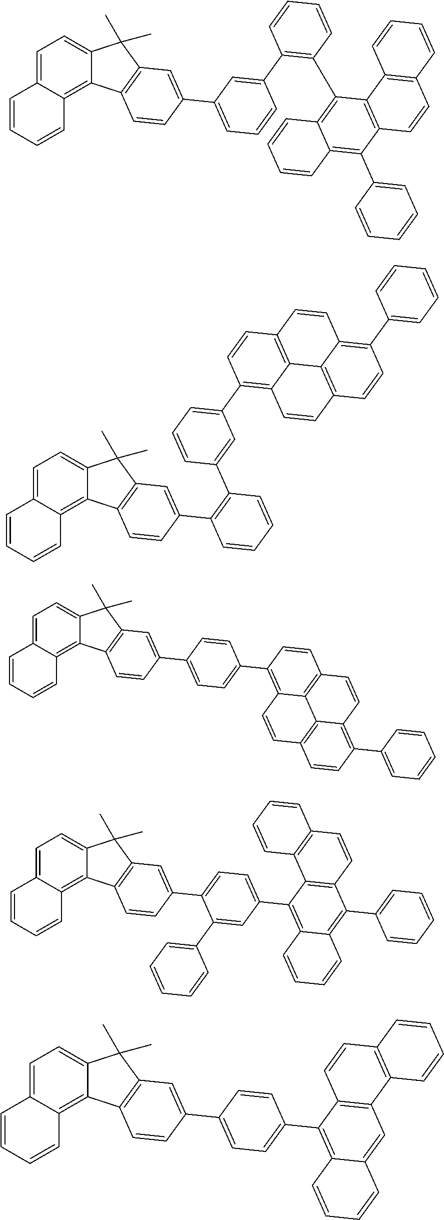

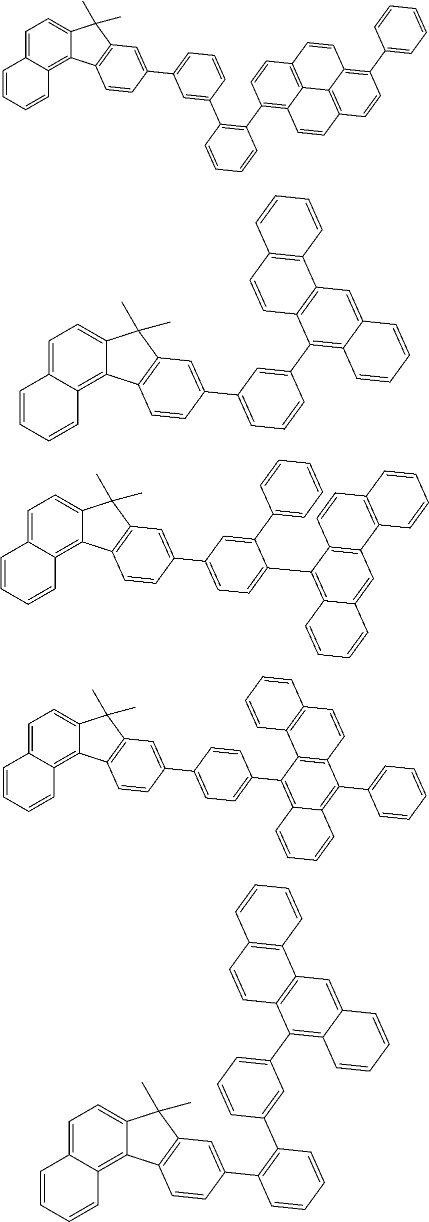

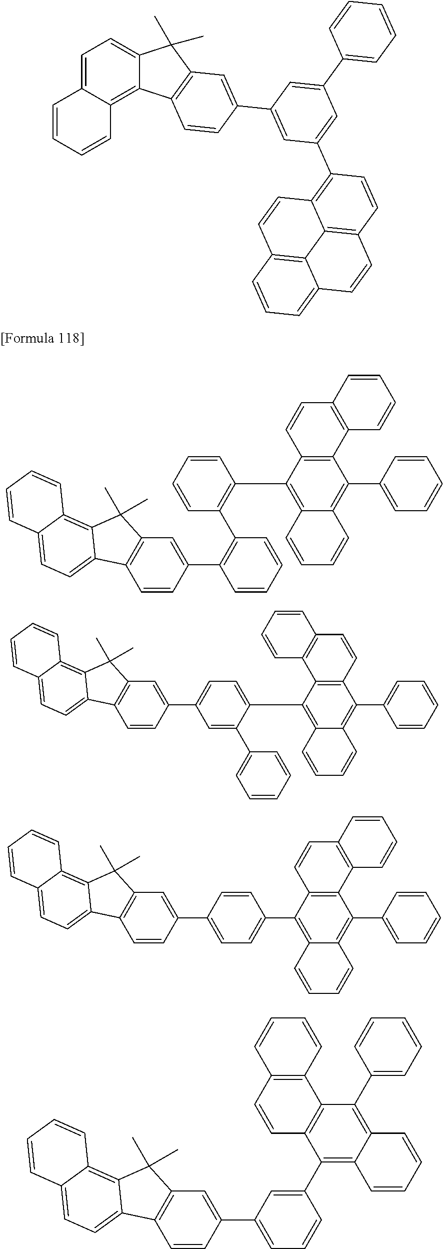

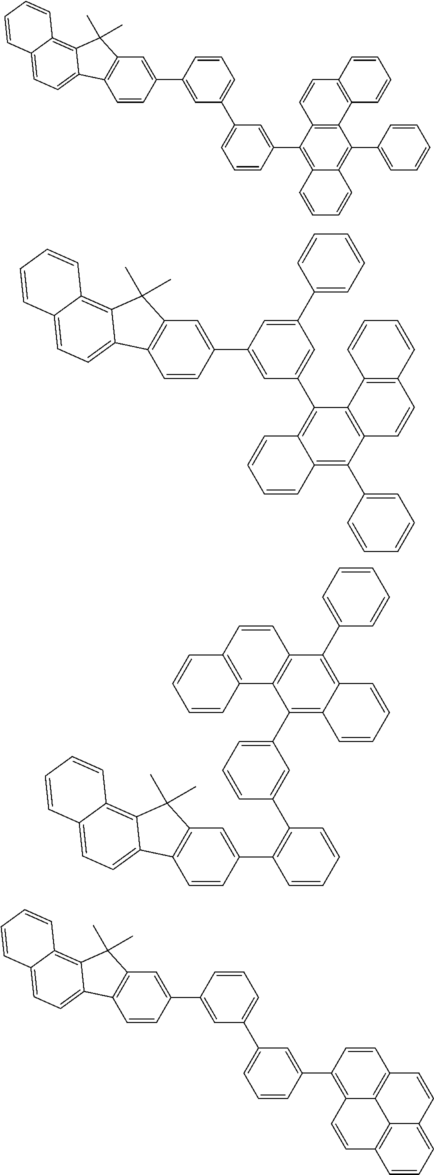

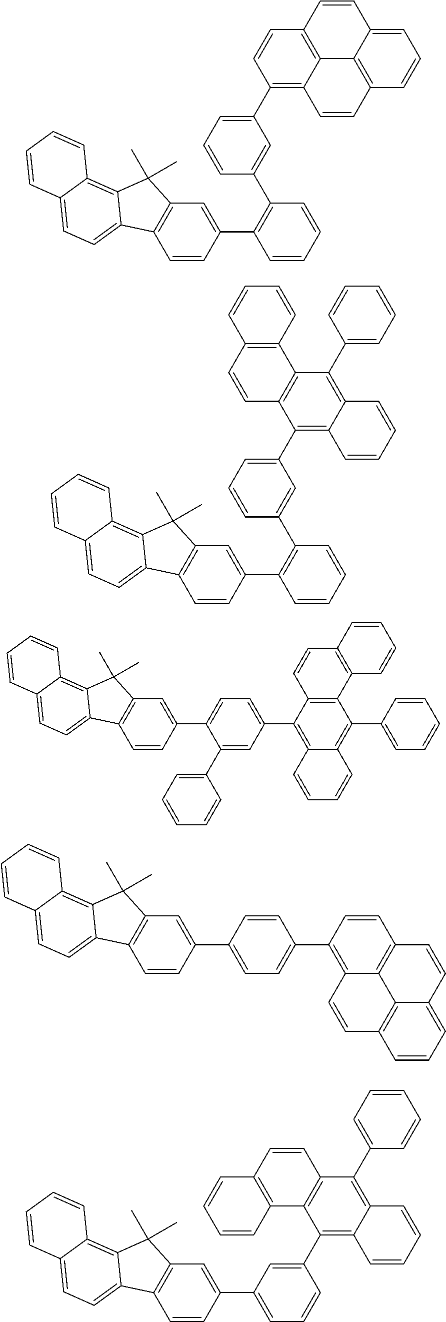

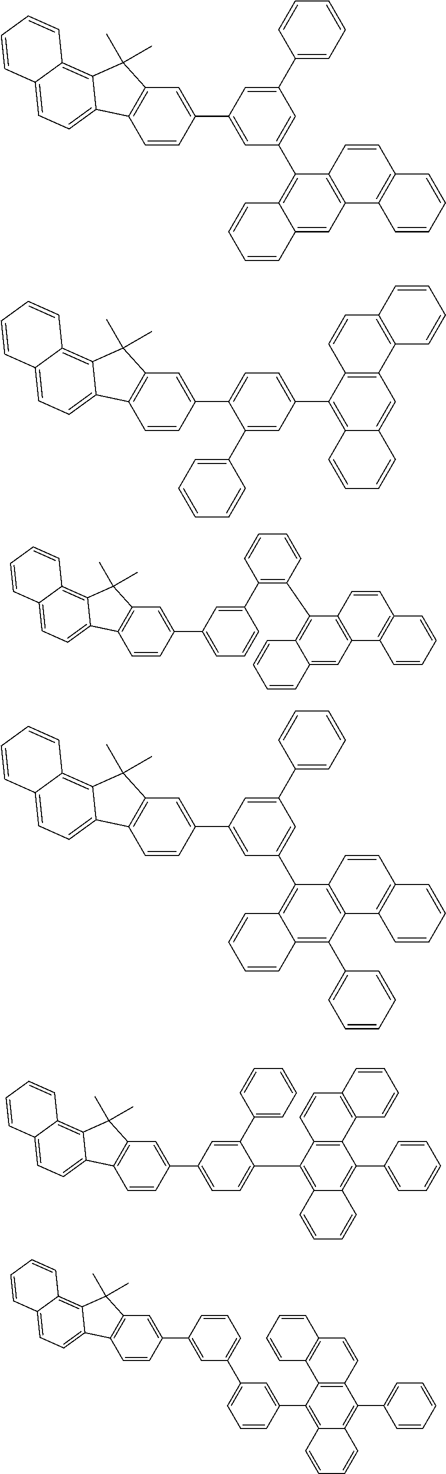

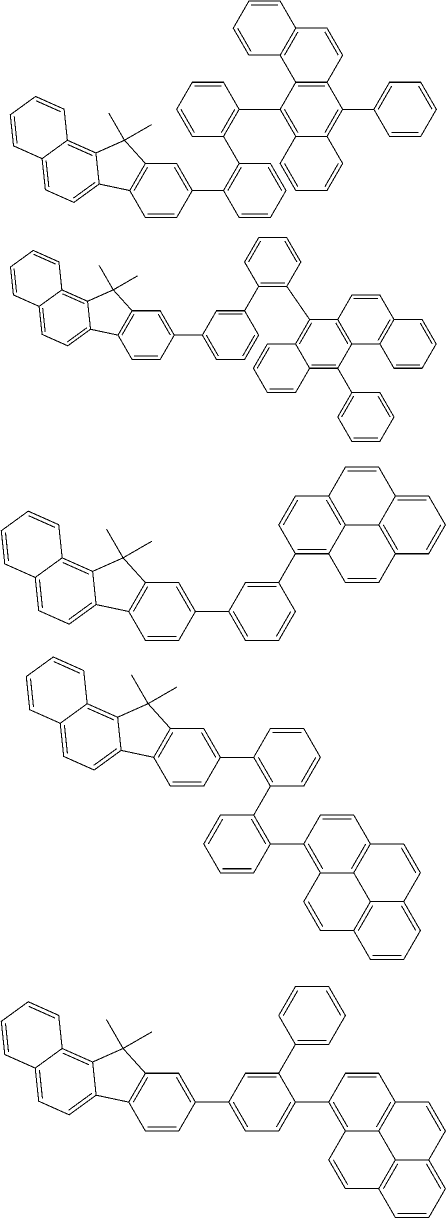

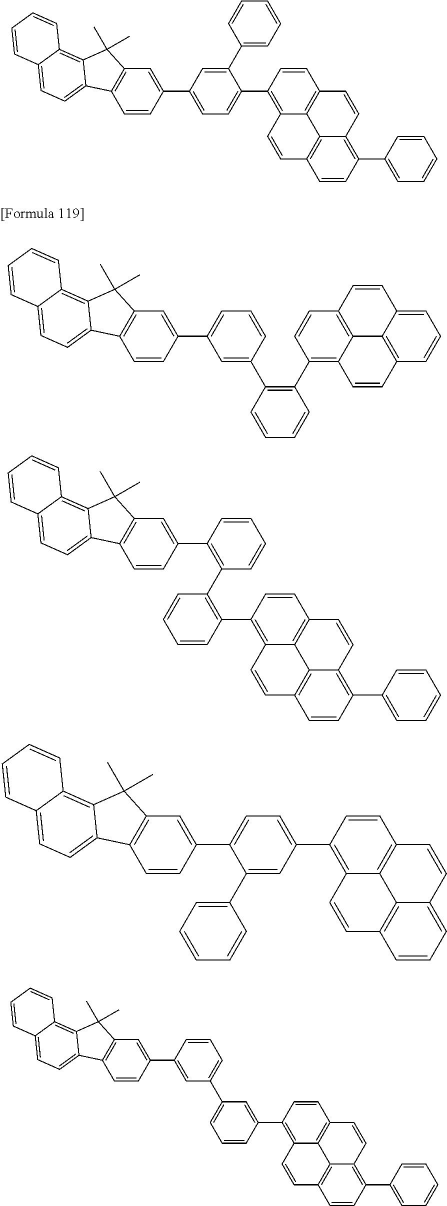

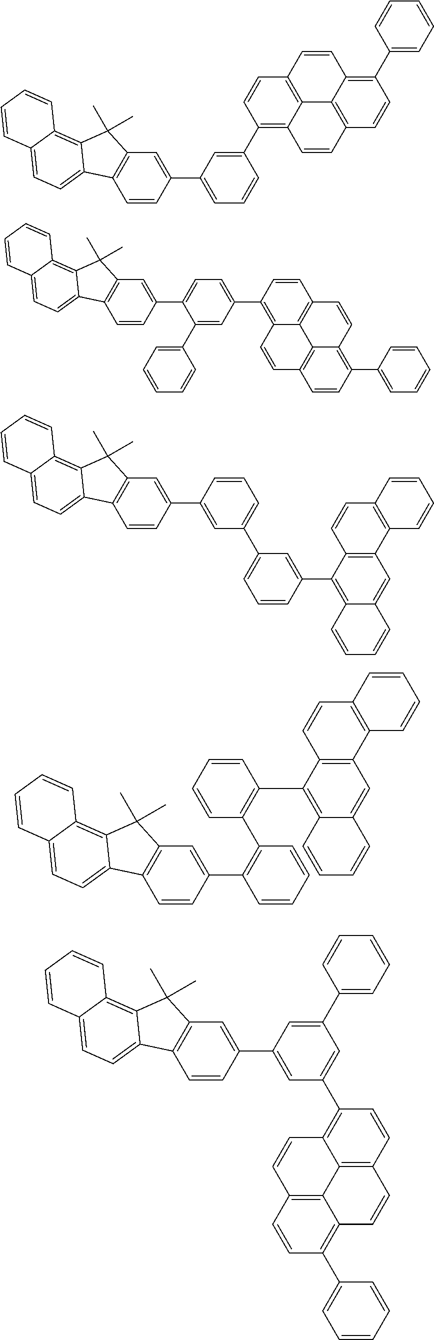

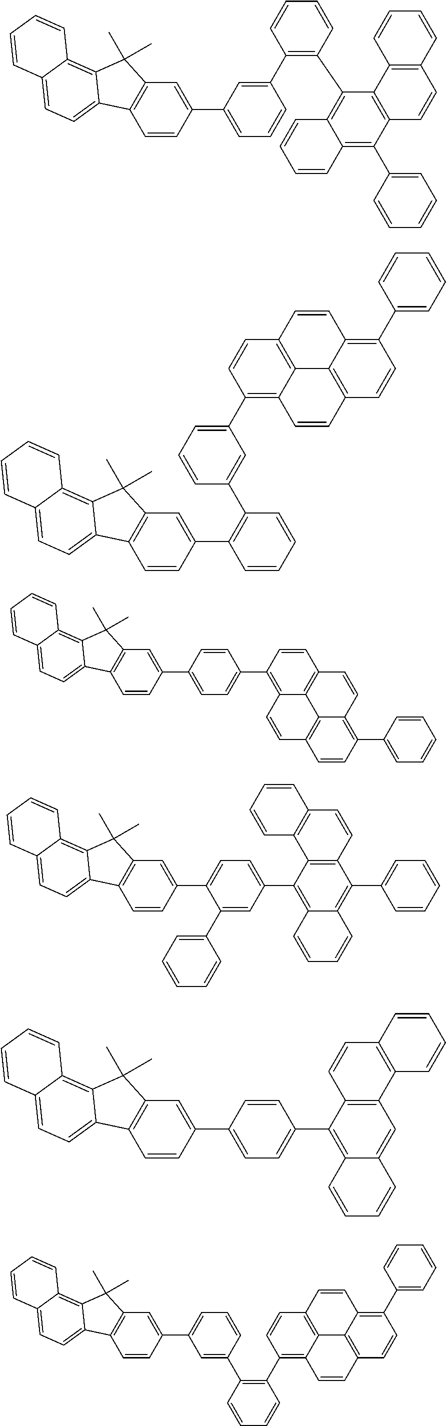

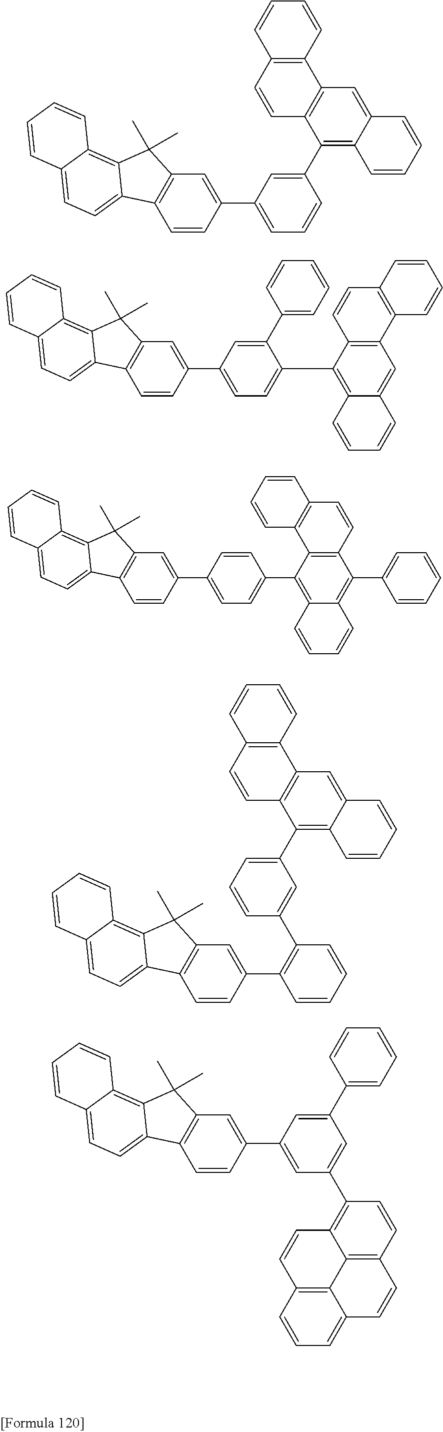

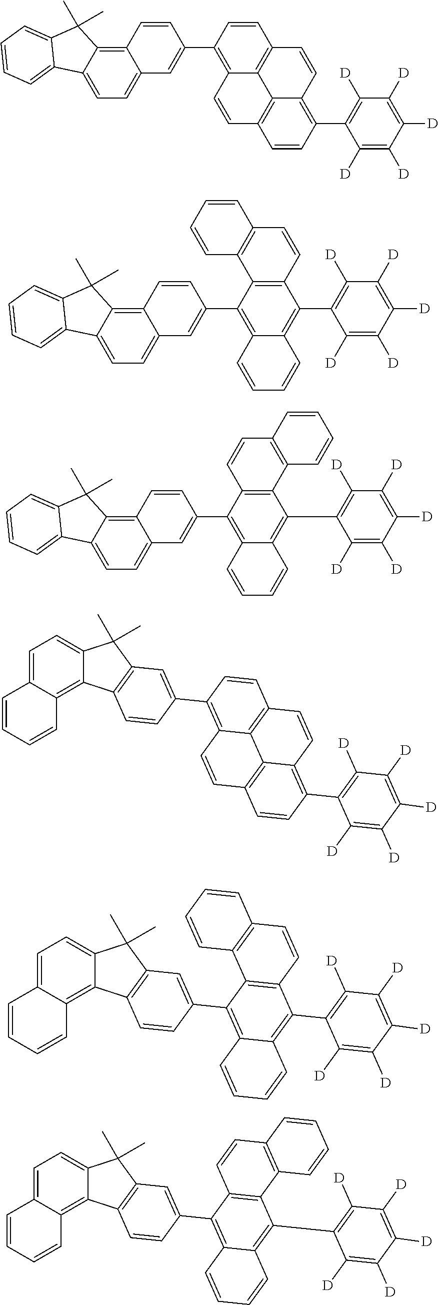

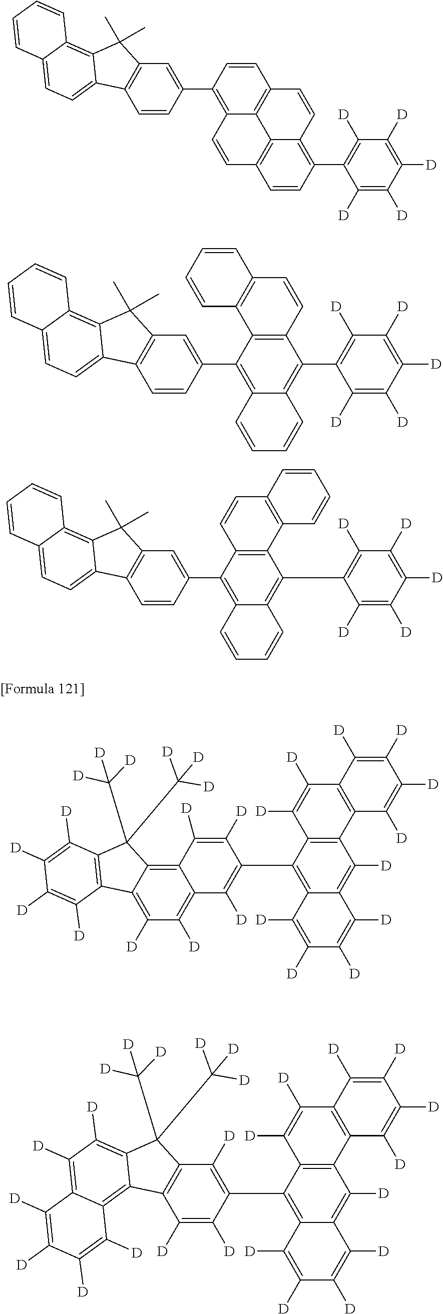

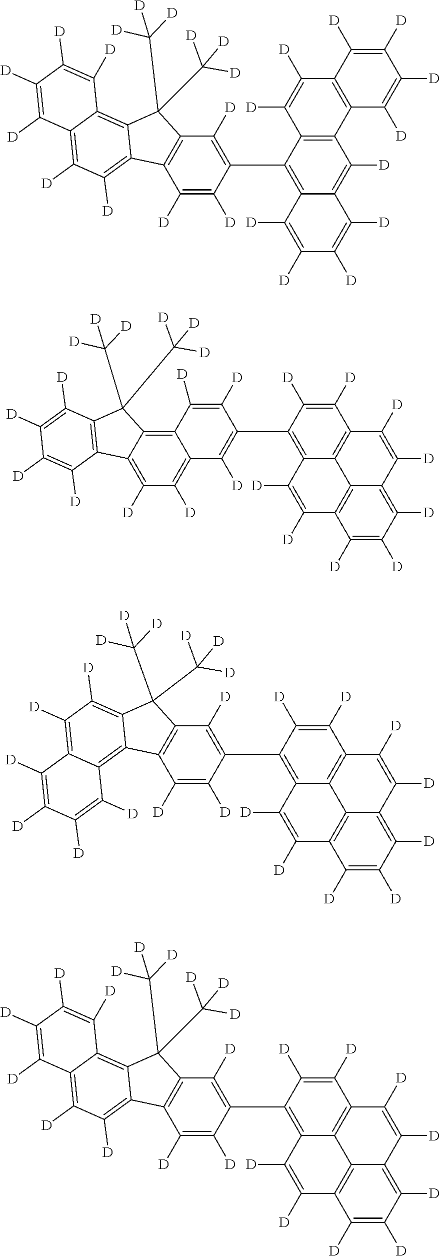

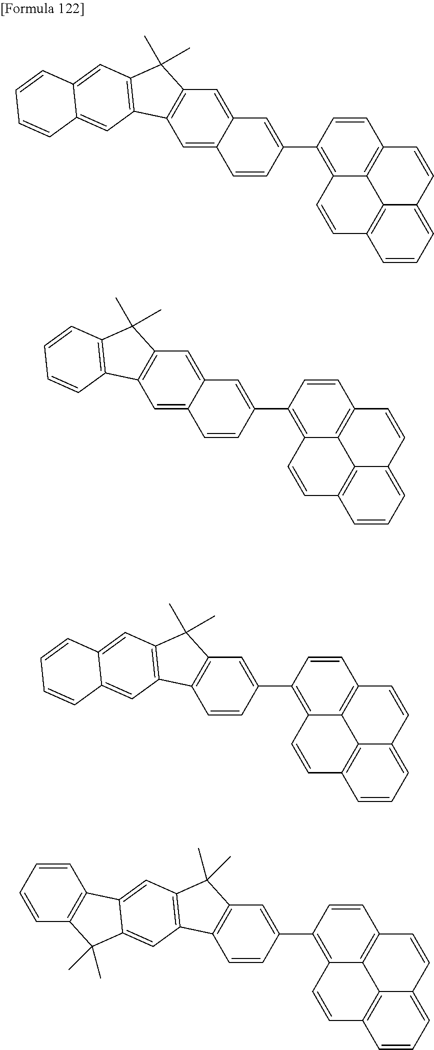

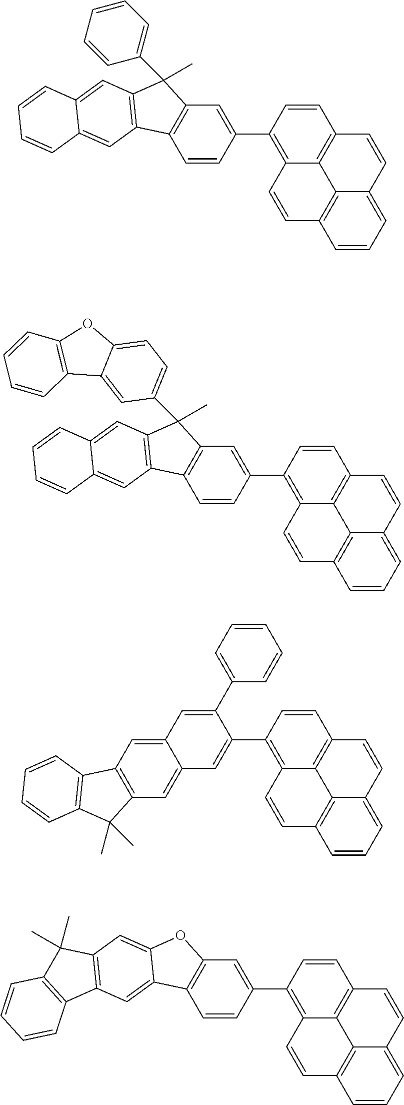

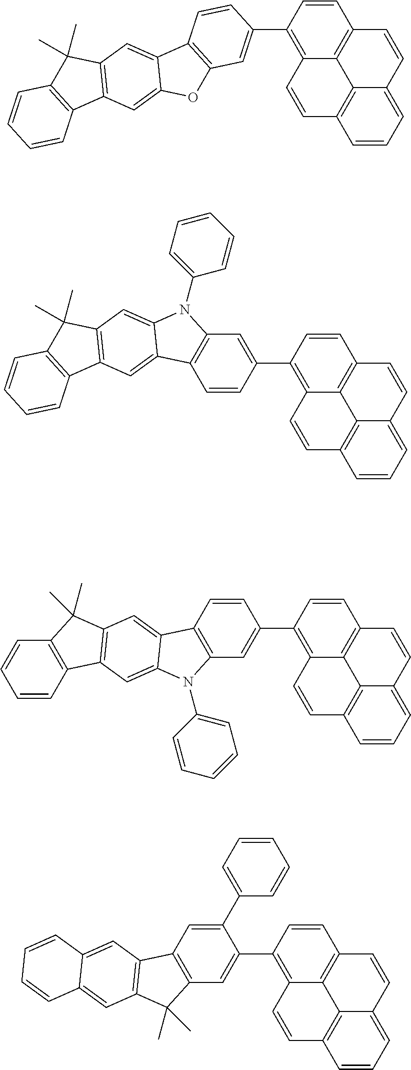

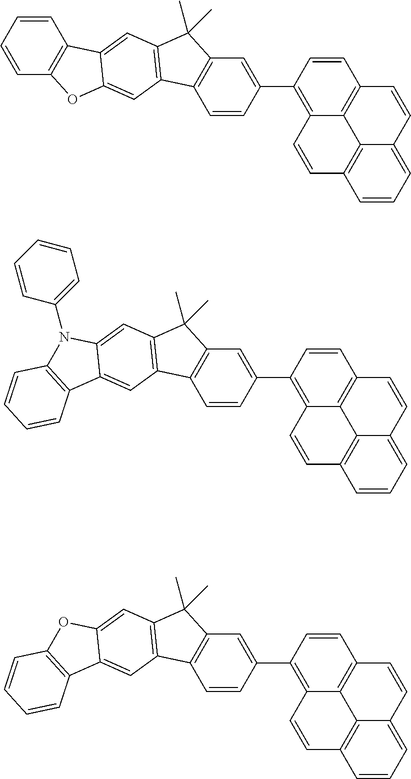 [SMILES: CC1(C)c2cc(-c3ccc4ccc5cccc6ccc3c4c56)ccc2-c2cc3oc4ccccc4c3cc21 ; CC1(C)c2cc(-c3ccc4ccc5cccc6ccc3c4c56)ccc2-c2cc3c4ccccc4n(-c4ccccc4)c3cc21 ; CC1(C)c2cc(-c3ccc4ccc5cccc6ccc3c4c56)ccc2-c2cc3c(cc21)oc1ccccc13]

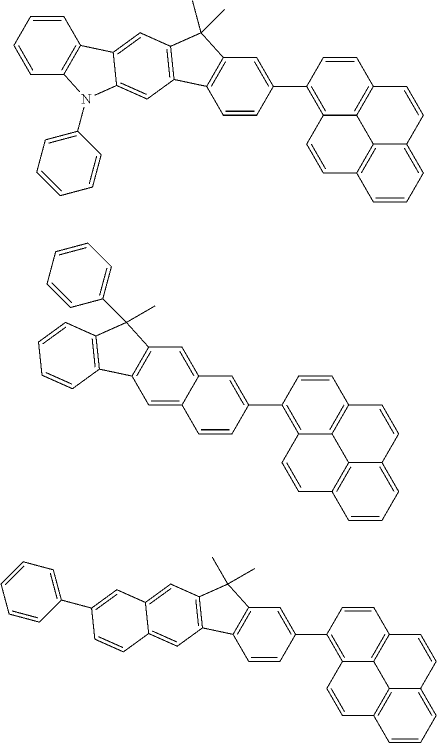

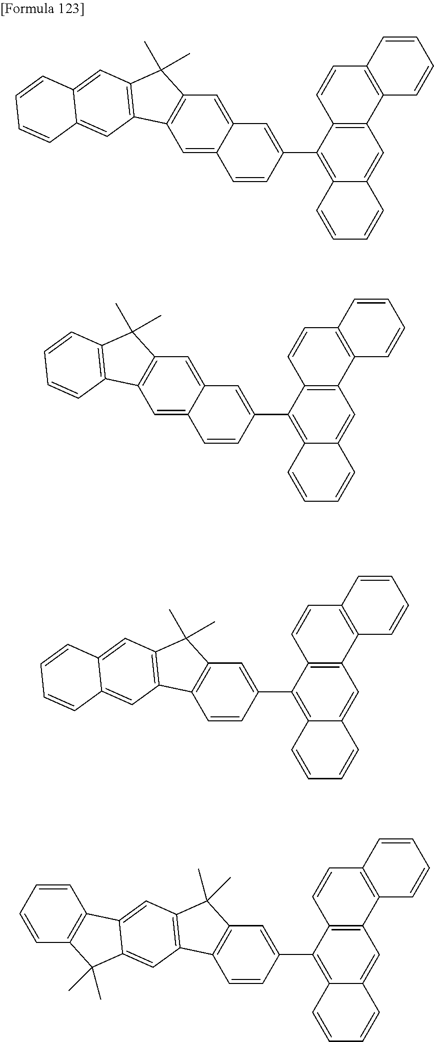

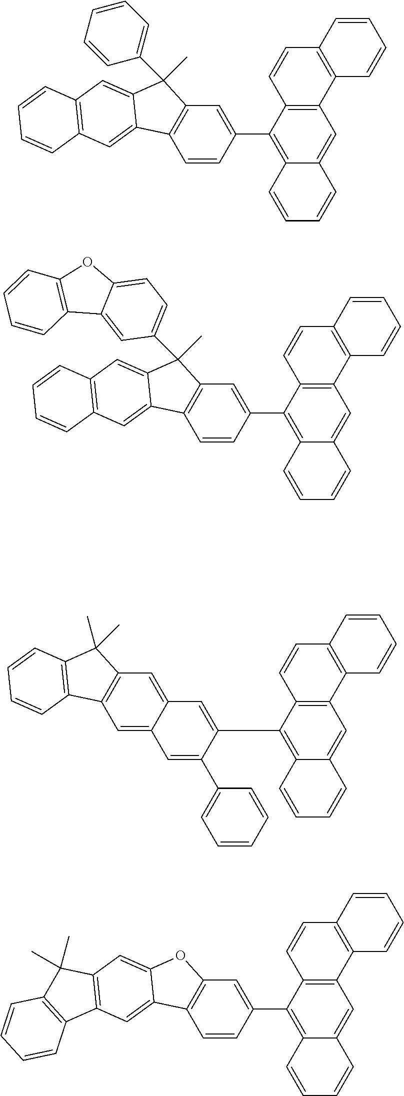

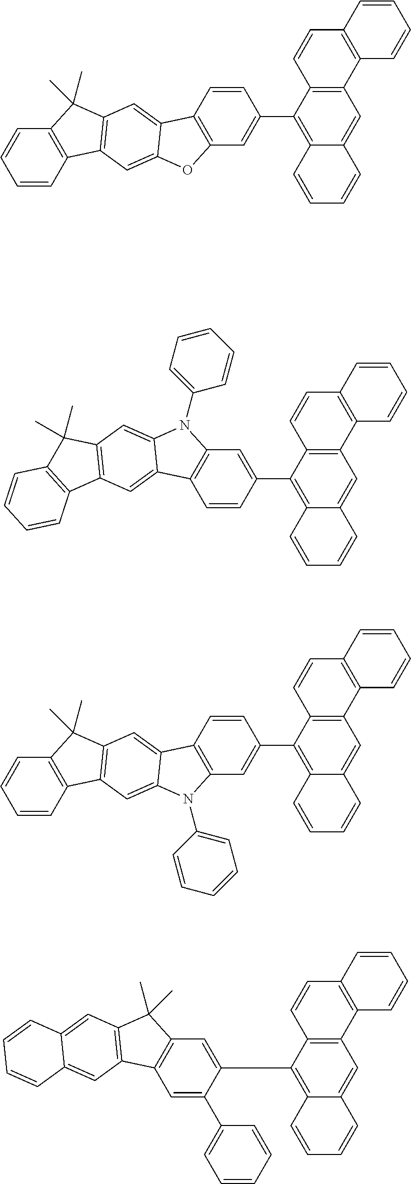

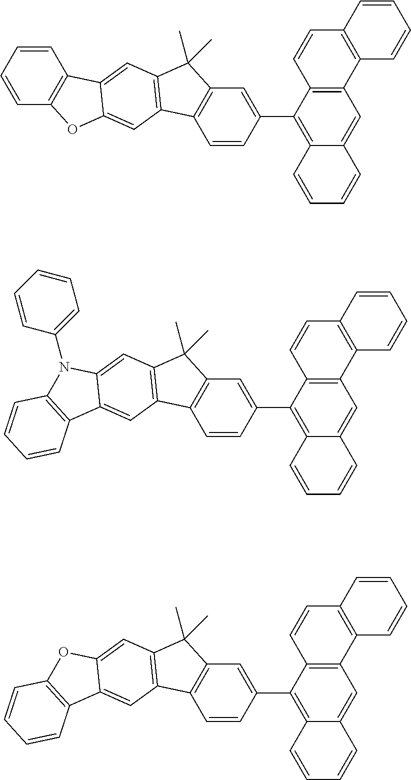 [SMILES: CC1(C)c2cc(-c3c4ccccc4cc4c3ccc3ccccc34)ccc2-c2cc3oc4ccccc4c3cc21 ; CC1(C)c2cc(-c3c4ccccc4cc4c3ccc3ccccc34)ccc2-c2cc3c4ccccc4n(-c4ccccc4)c3cc21 ; CC1(C)c2cc(-c3c4ccccc4cc4c3ccc3ccccc34)ccc2-c2cc3c(cc21)oc1ccccc13]

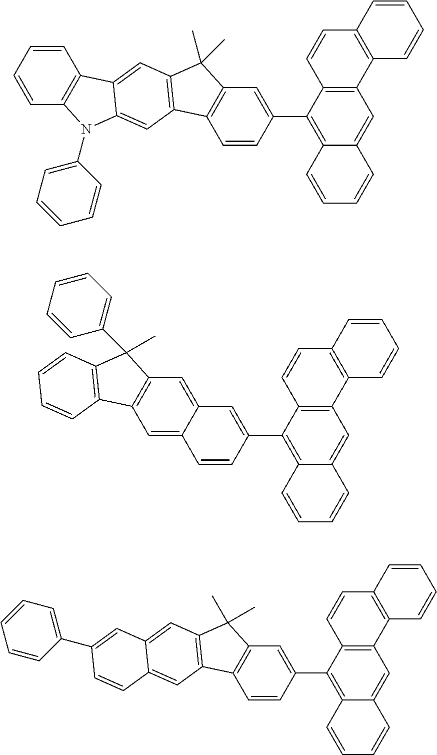

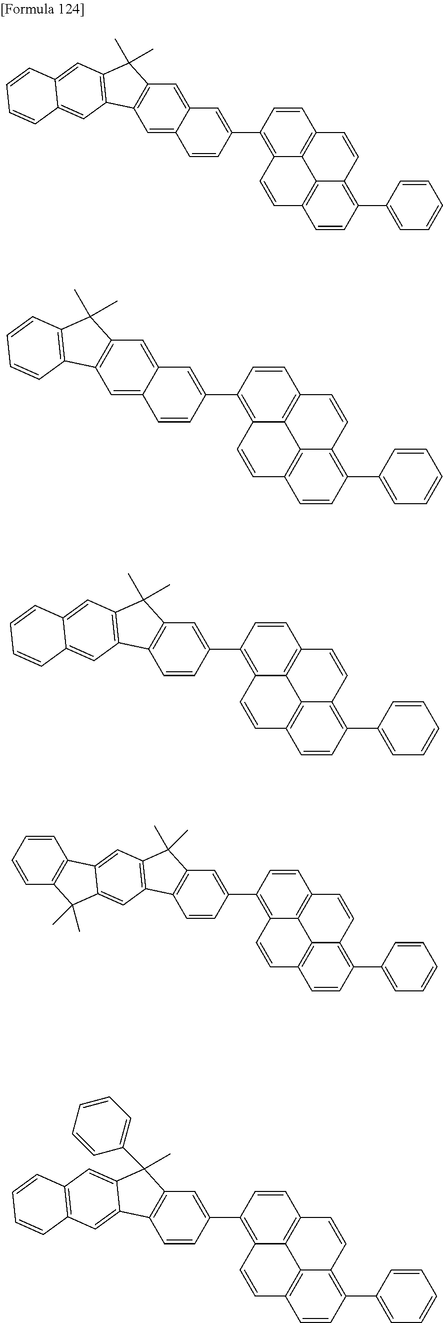

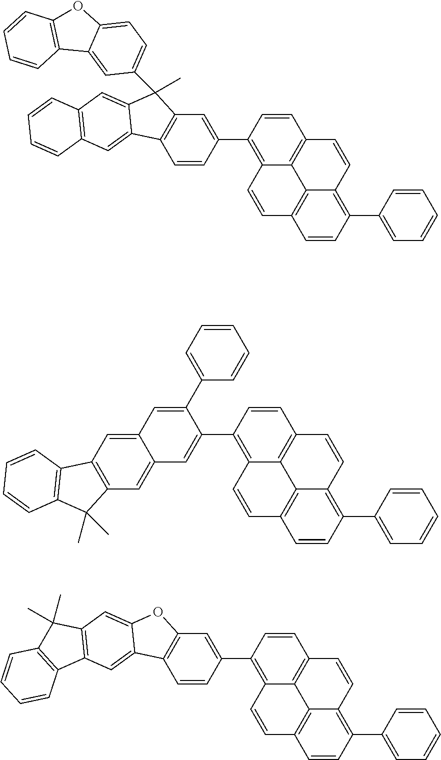

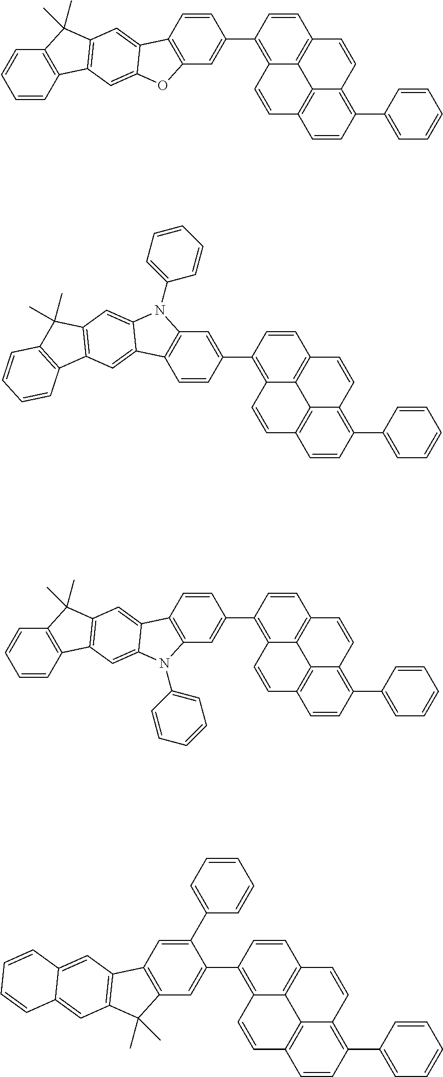

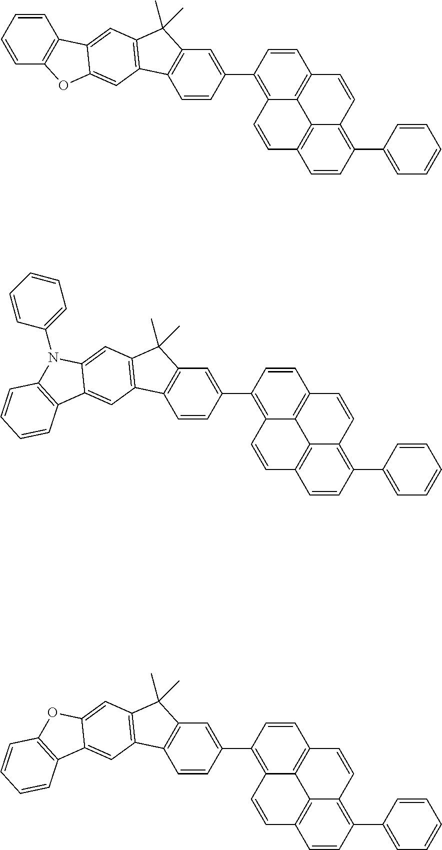 [SMILES: CC1(C)c2cc(-c3ccc4ccc5c(-c6ccccc6)ccc6ccc3c4c65)ccc2-c2cc3oc4ccccc4c3cc21 ; CC1(C)c2cc(-c3ccc4ccc5c(-c6ccccc6)ccc6ccc3c4c65)ccc2-c2cc3c4ccccc4n(-c4ccccc4)c3cc21 ; CC1(C)c2cc(-c3ccc4ccc5c(-c6ccccc6)ccc6ccc3c4c65)ccc2-c2cc3c(cc21)oc1ccccc13]

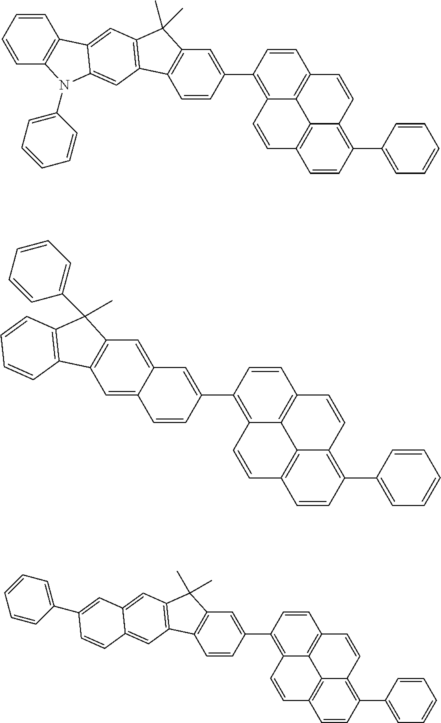

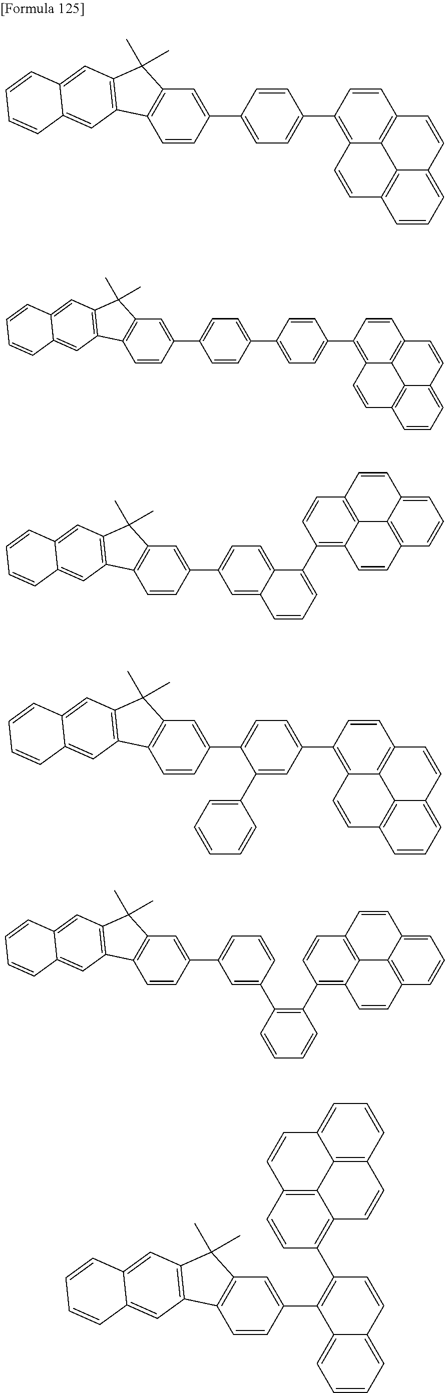

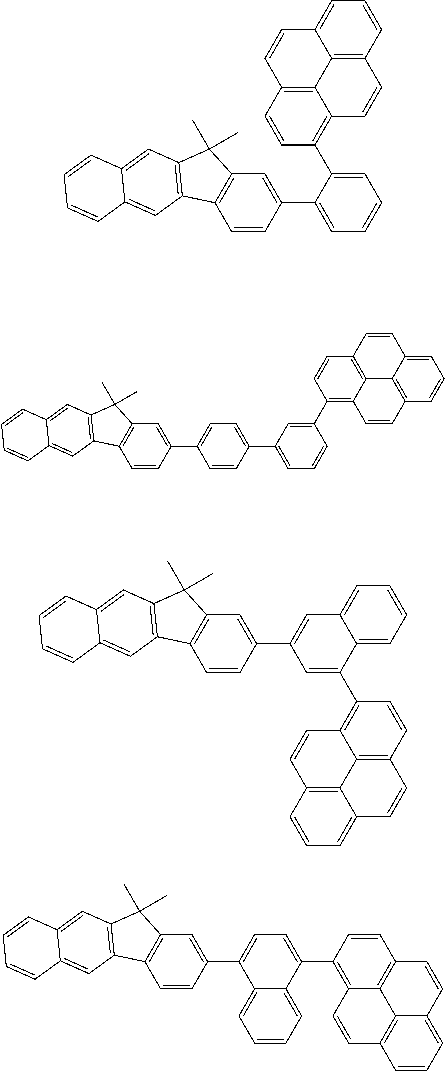

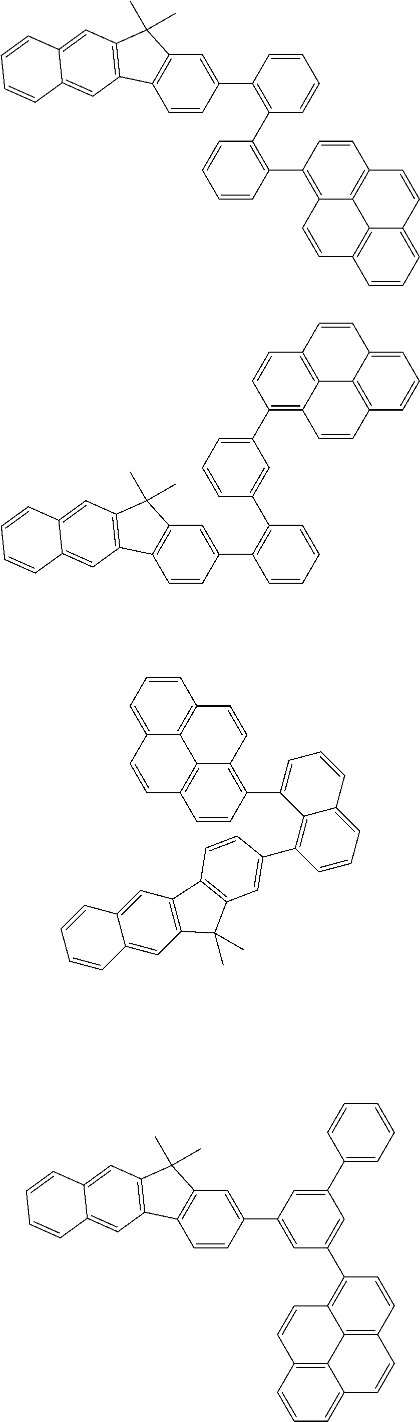

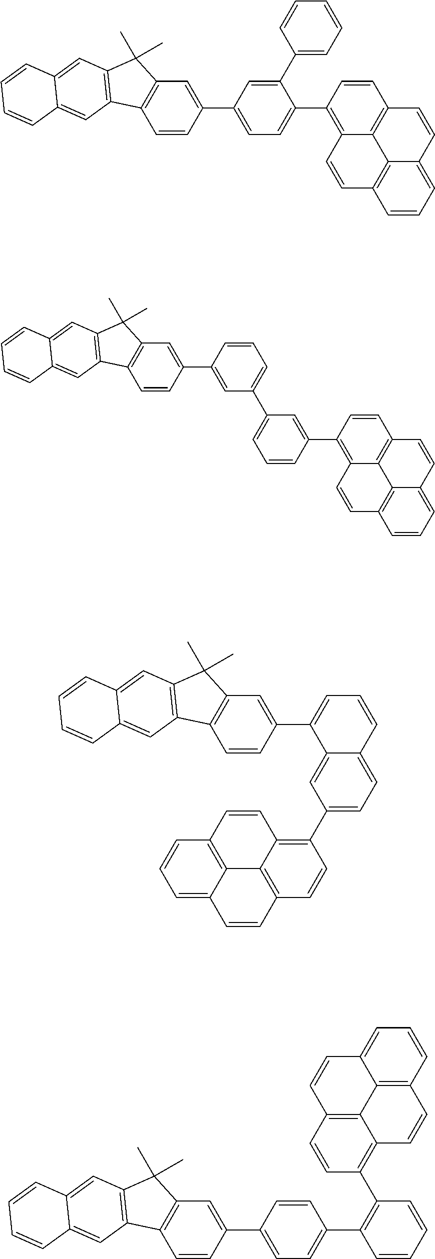

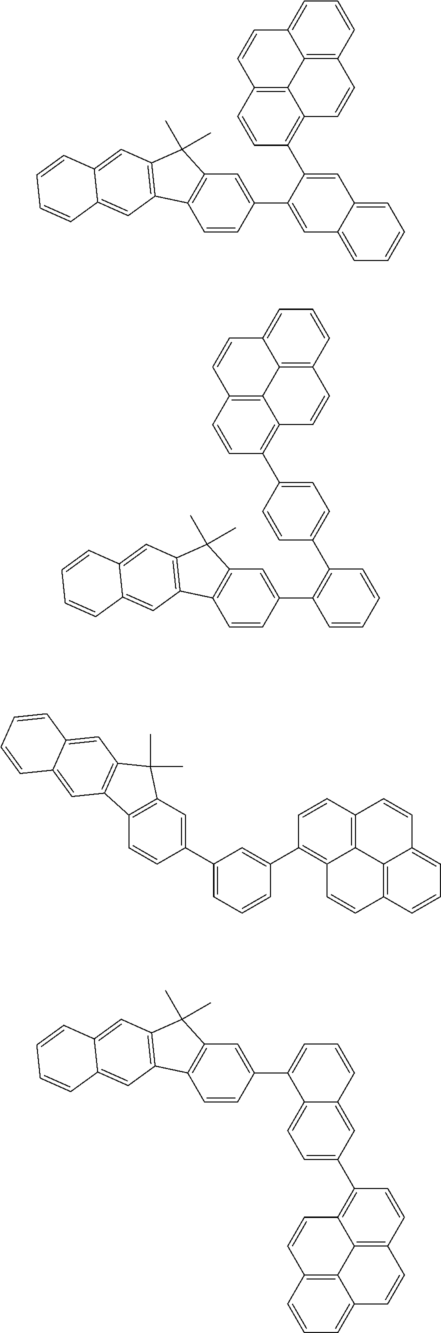

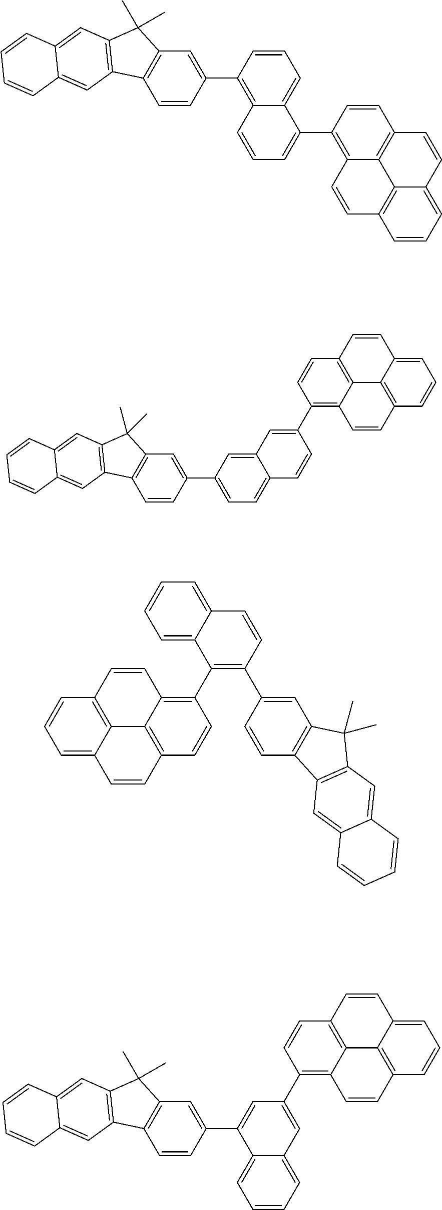

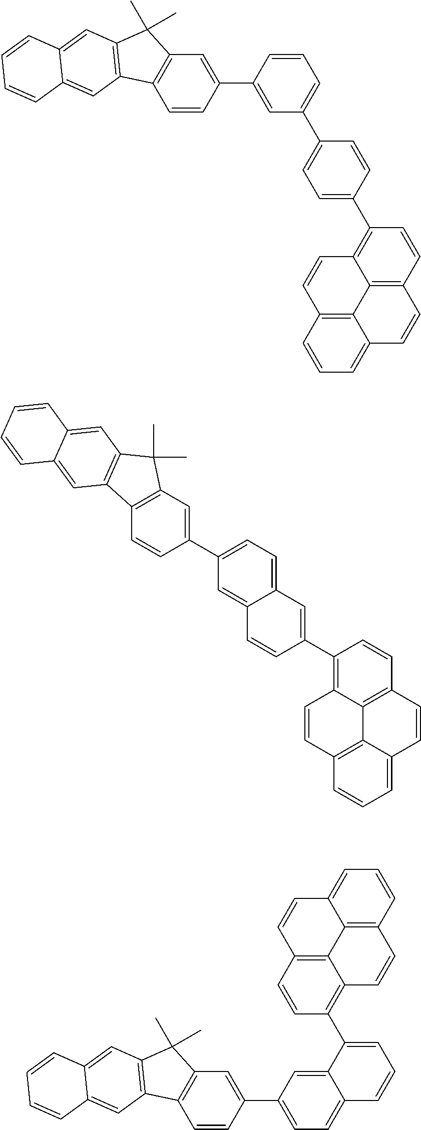

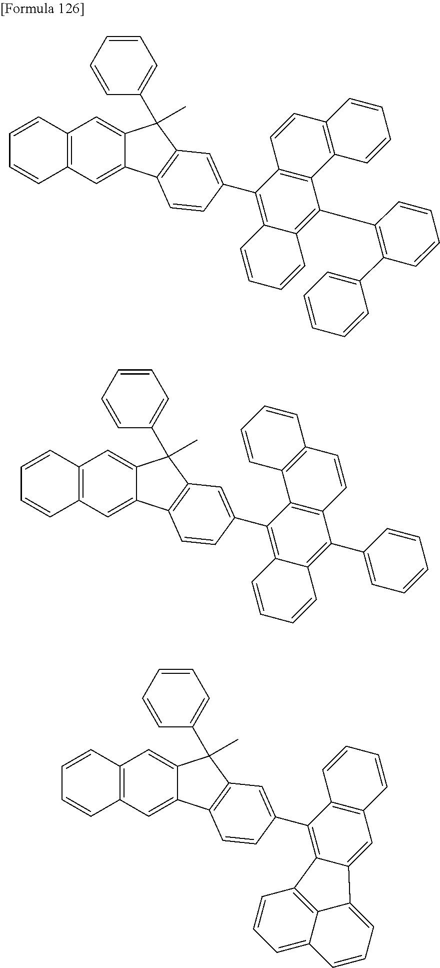

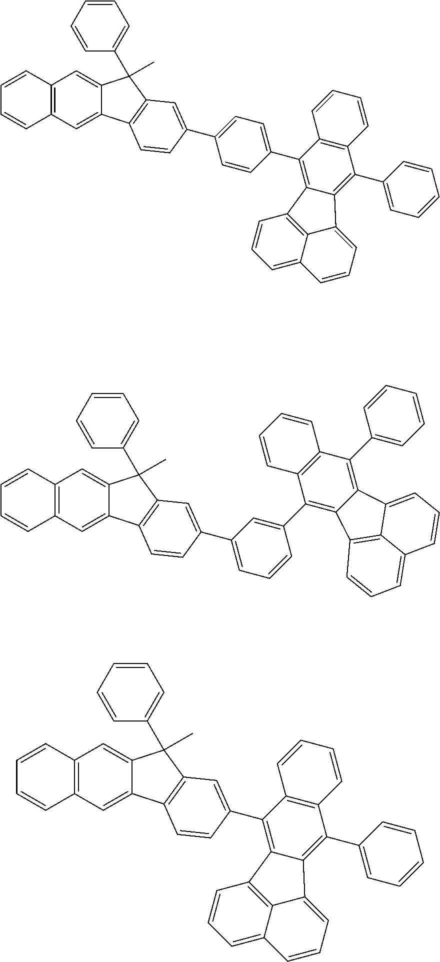

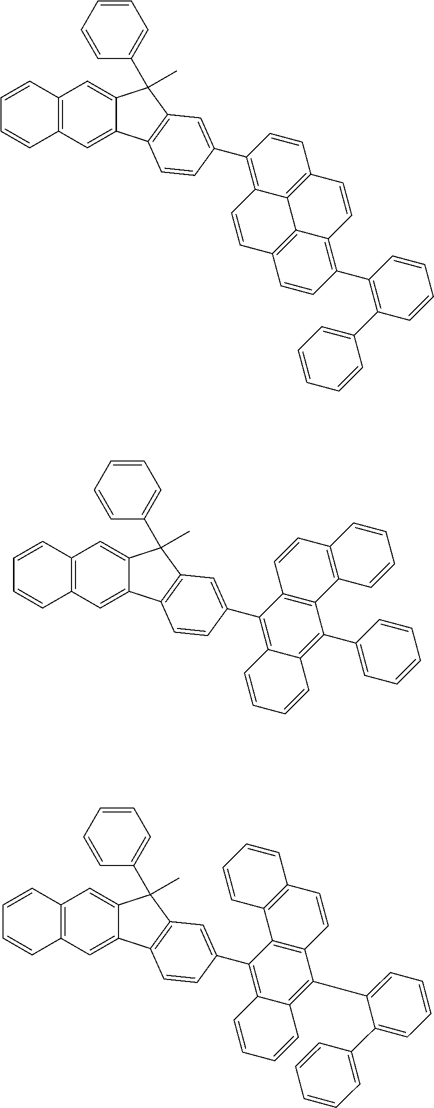

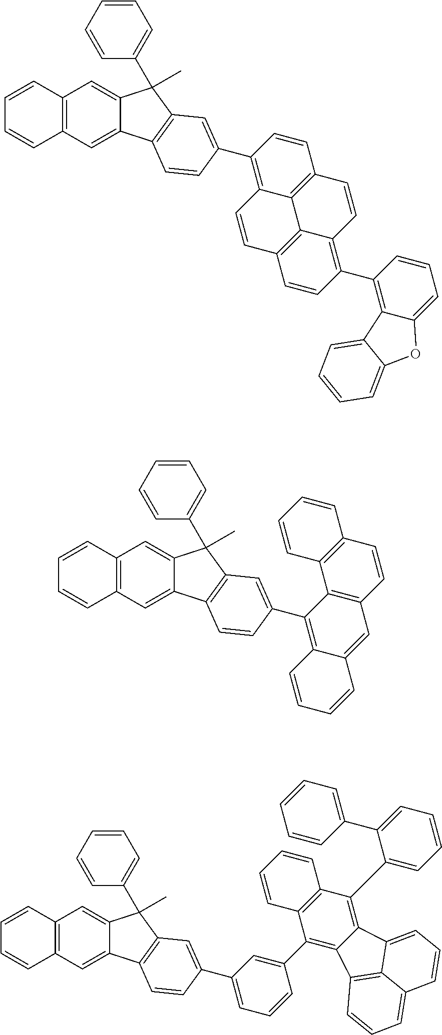

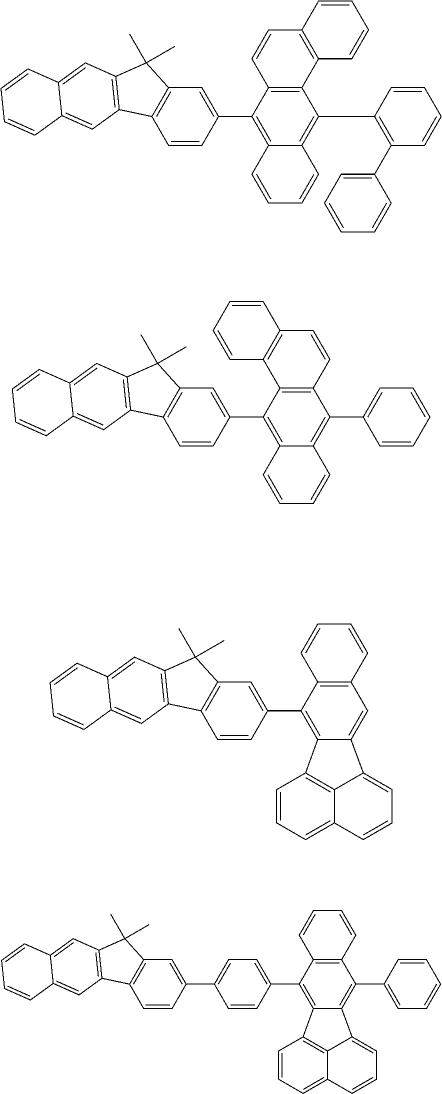

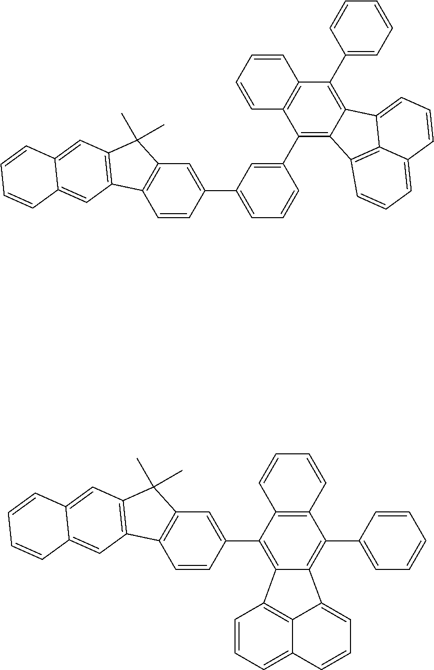

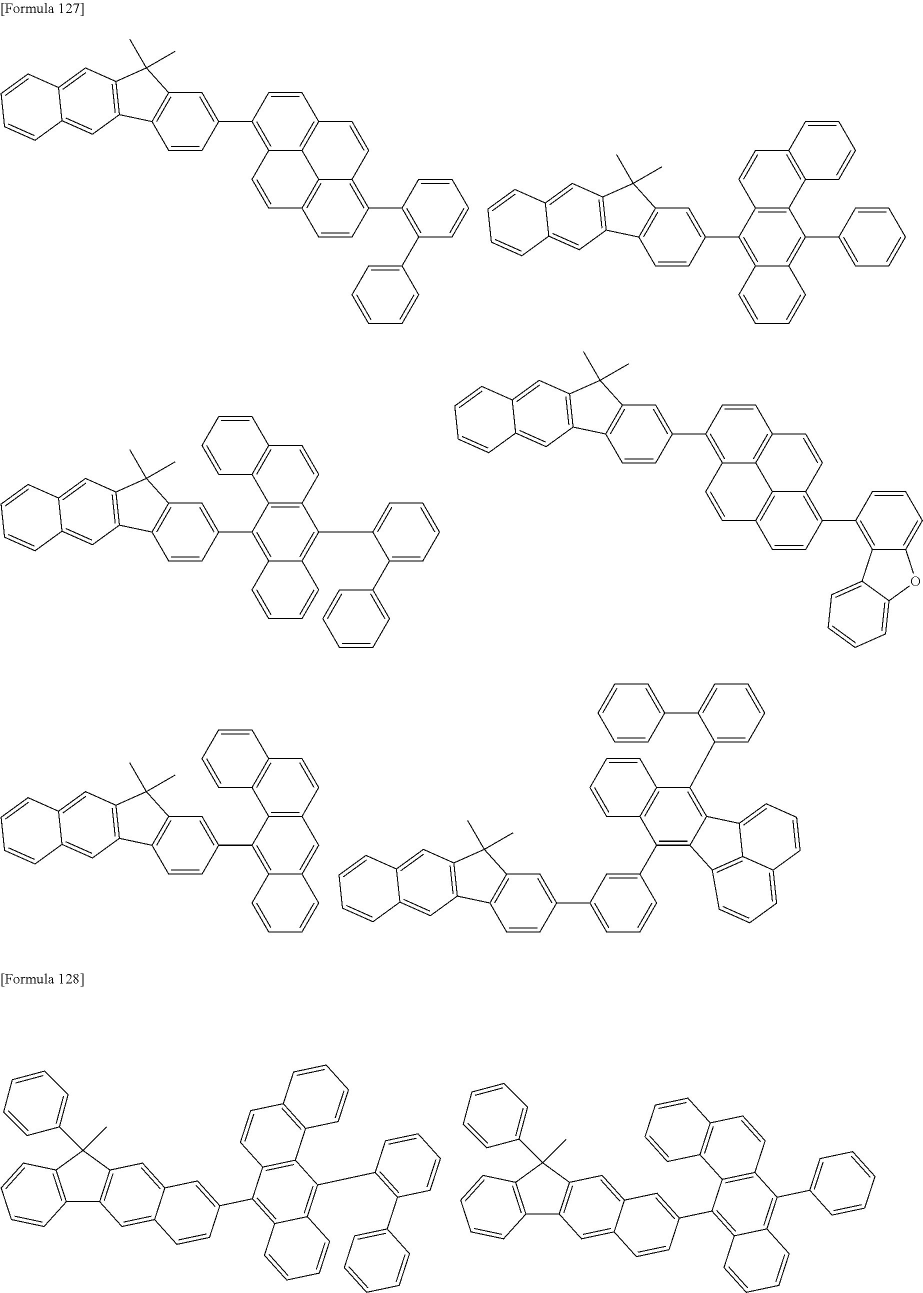

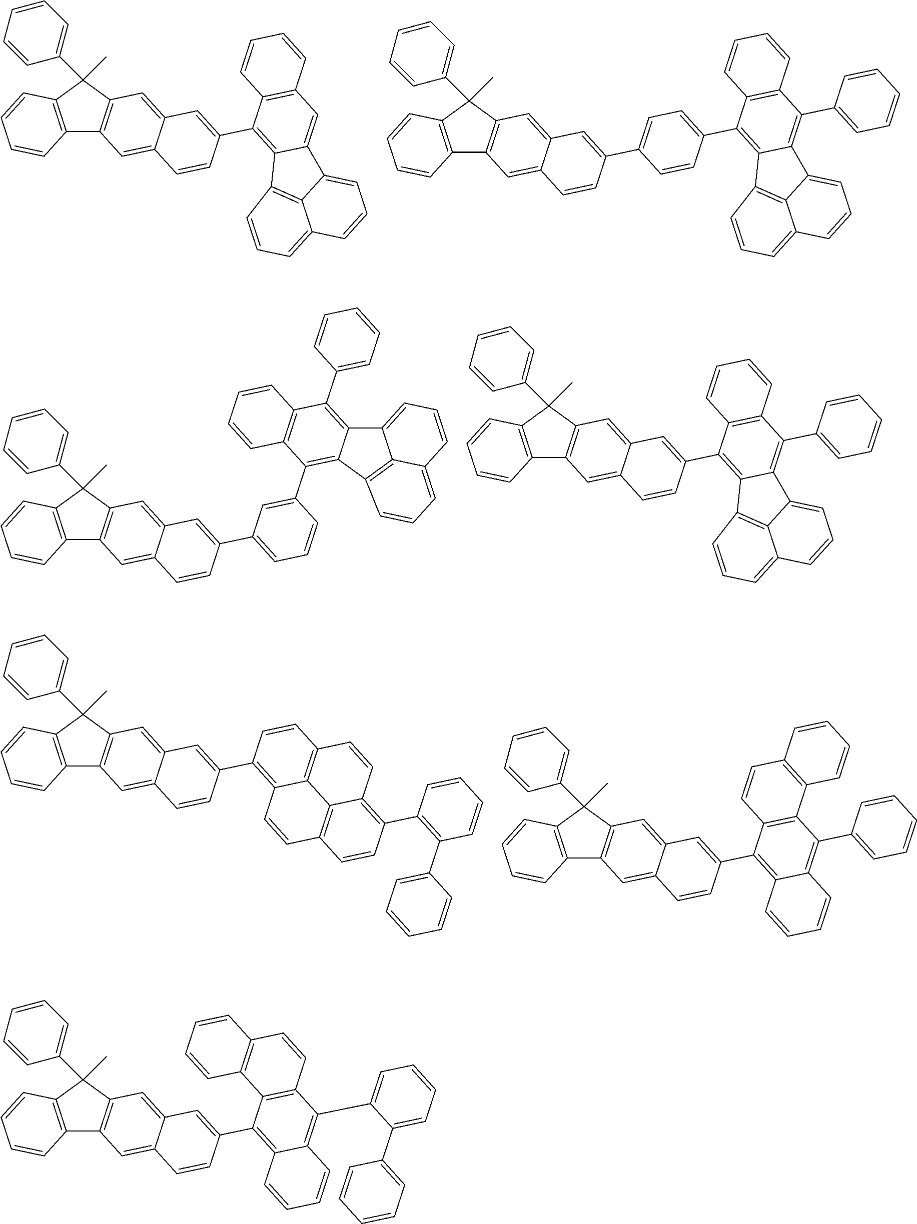

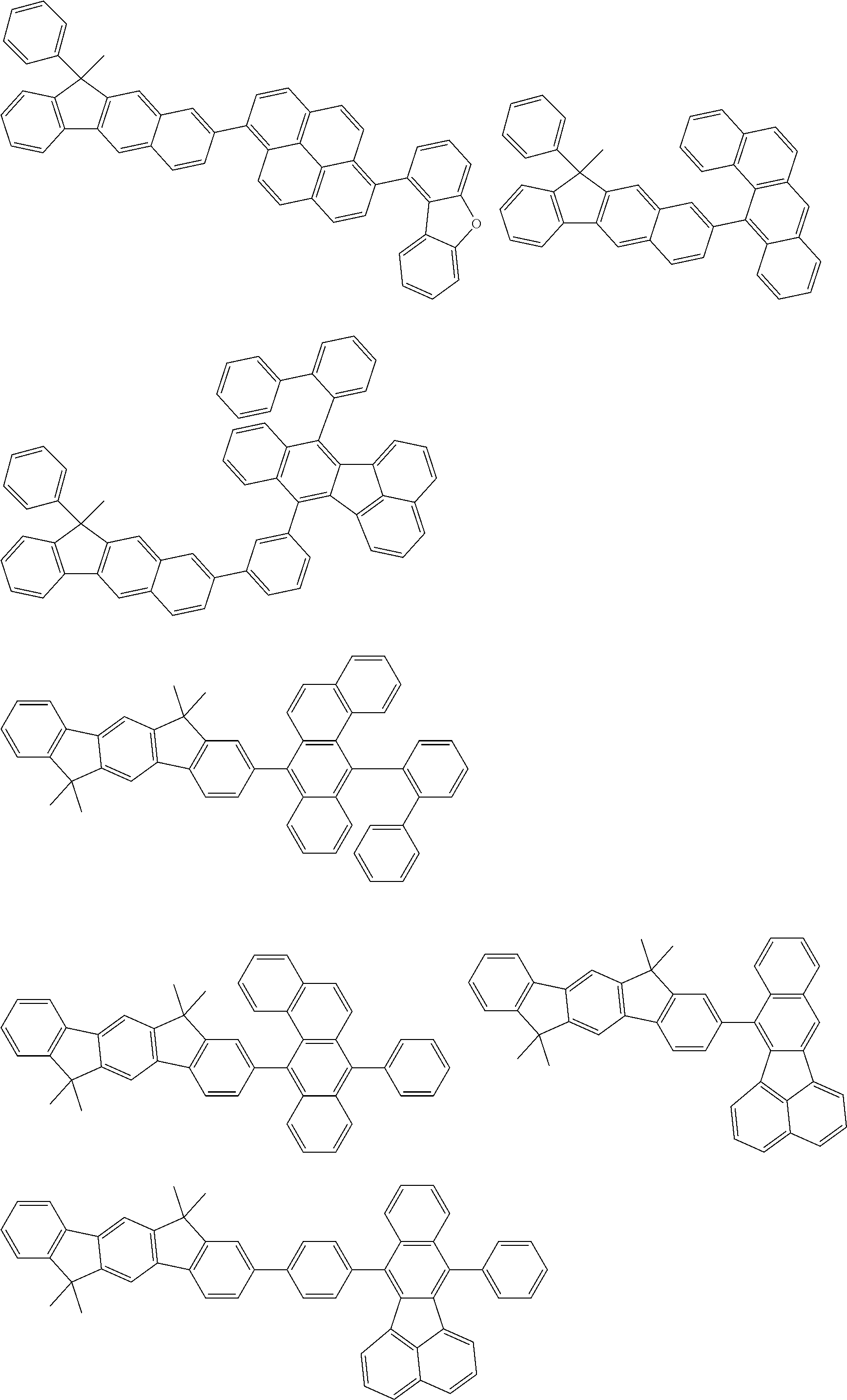

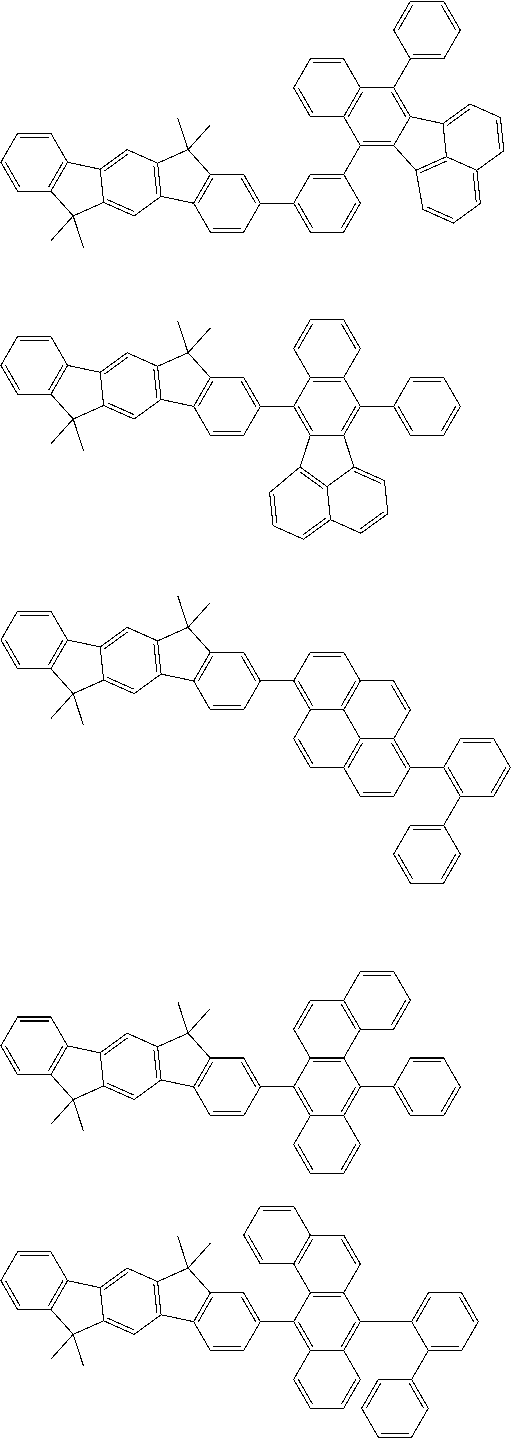

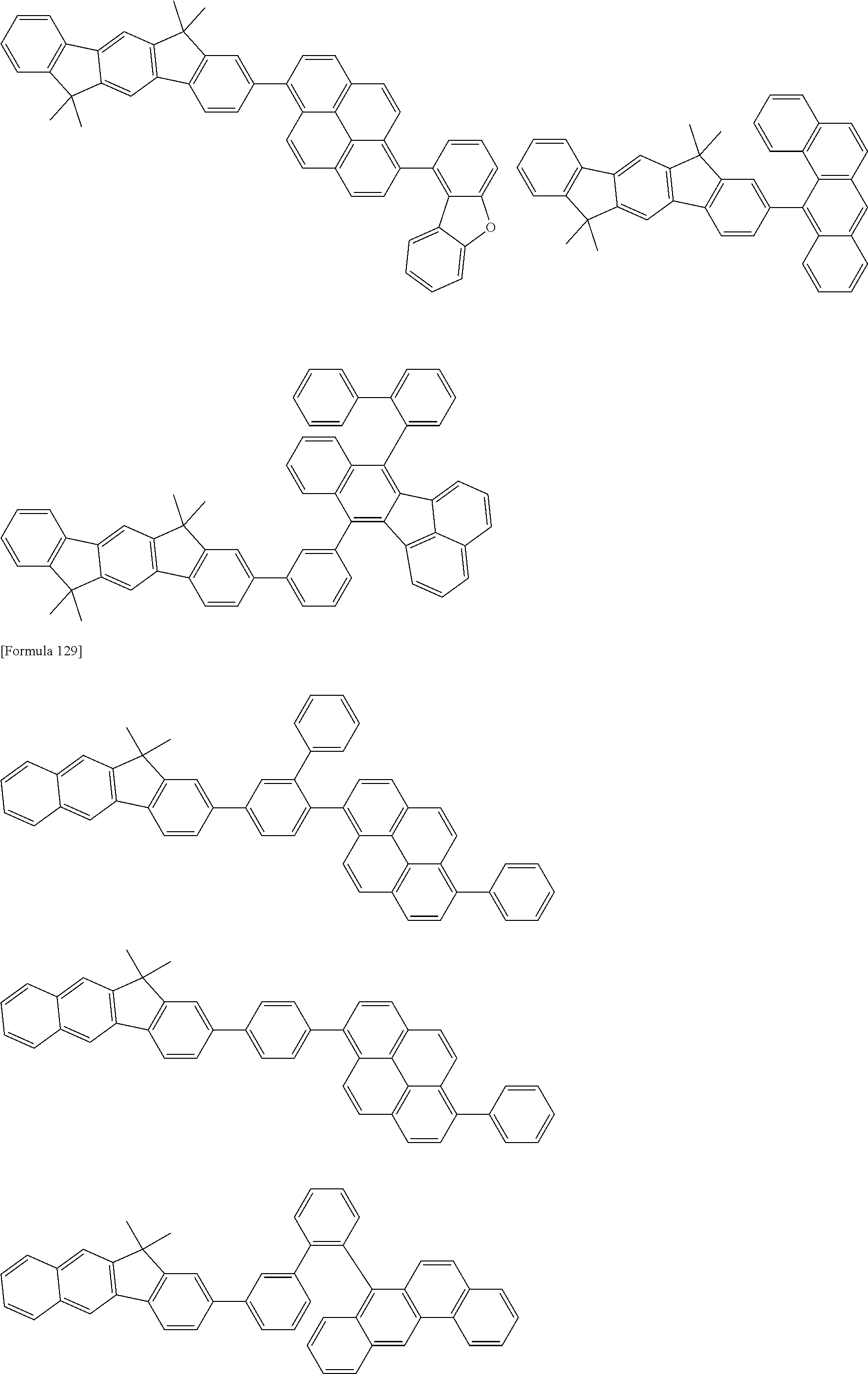

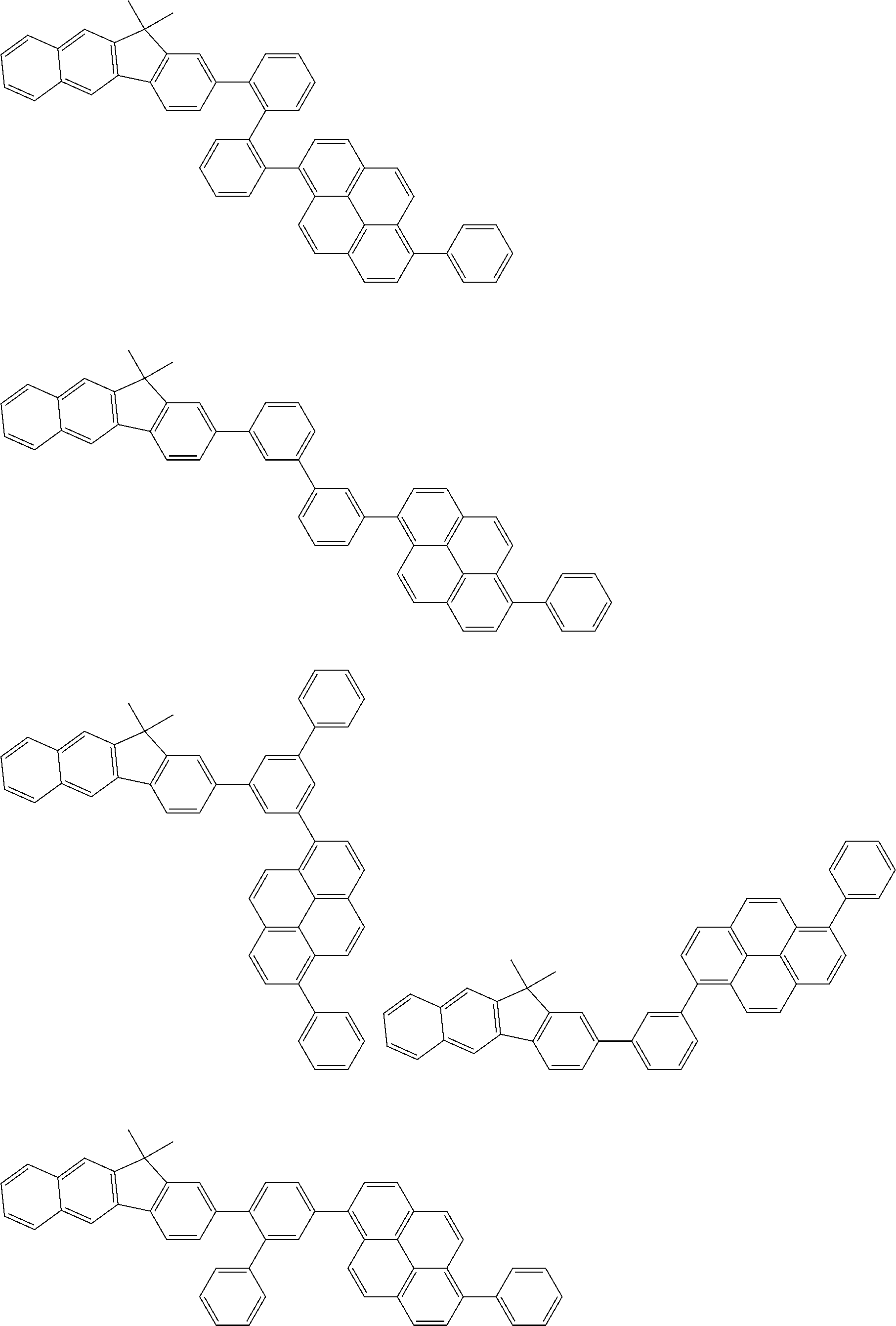

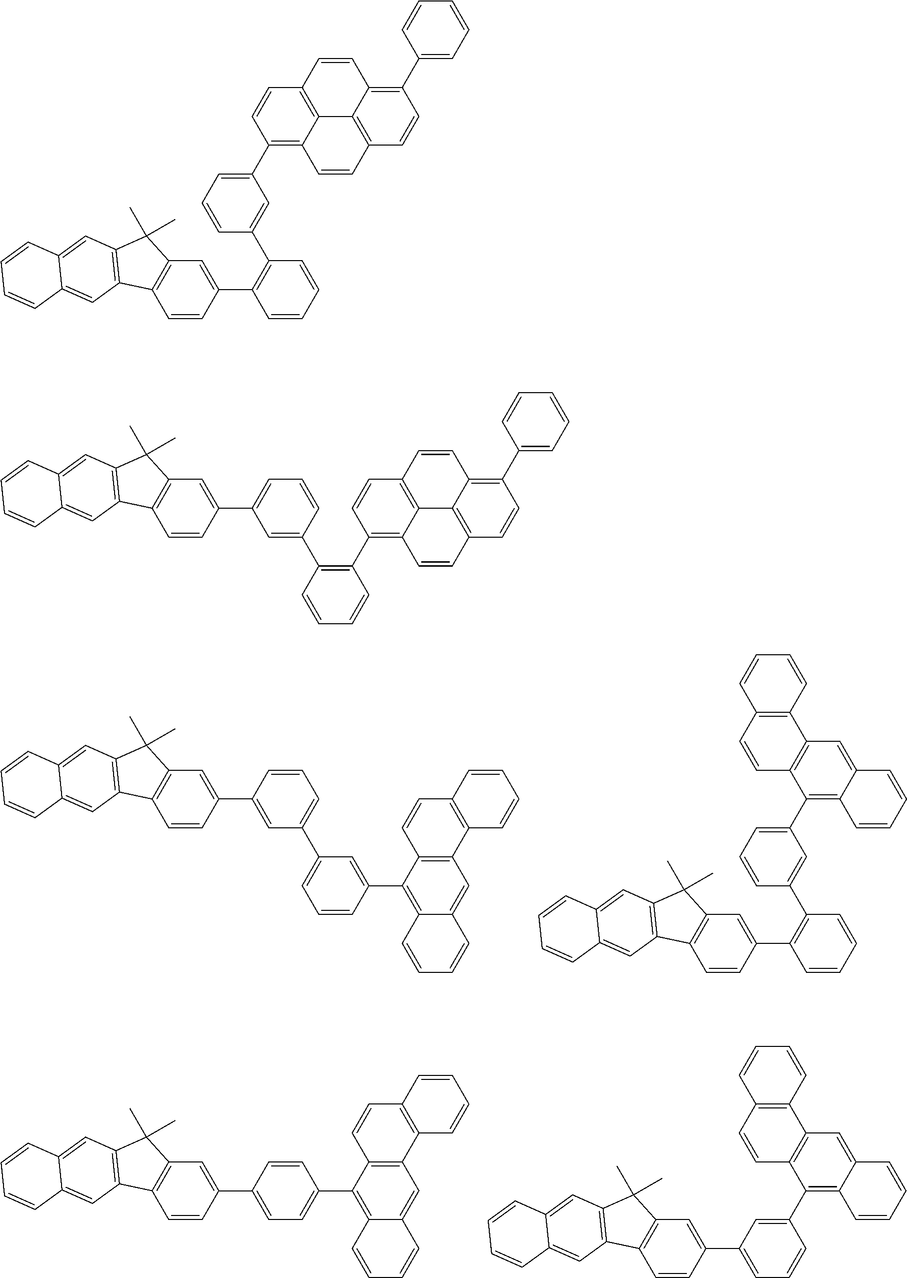

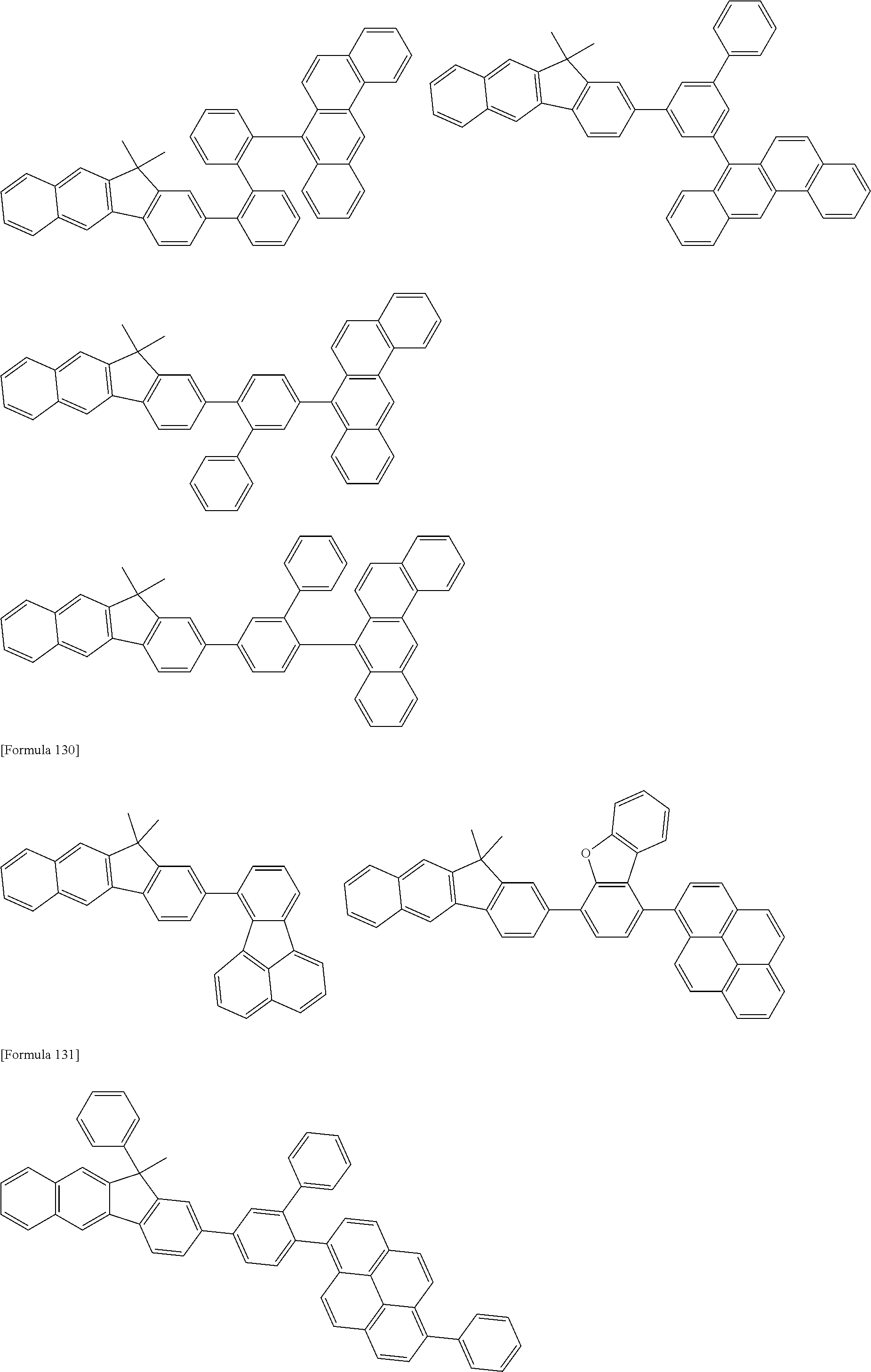

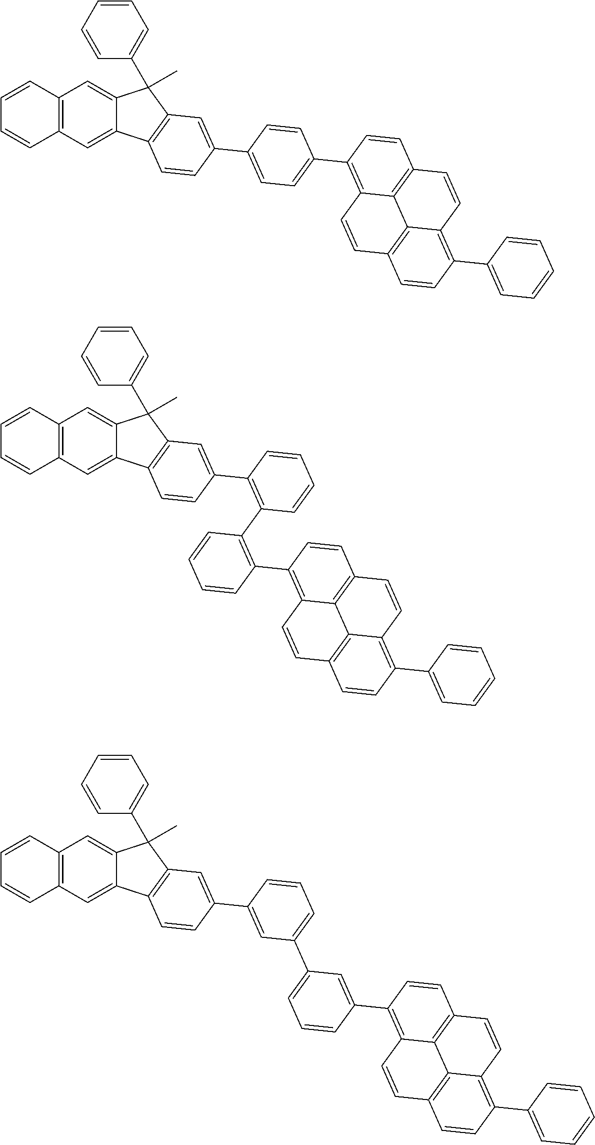

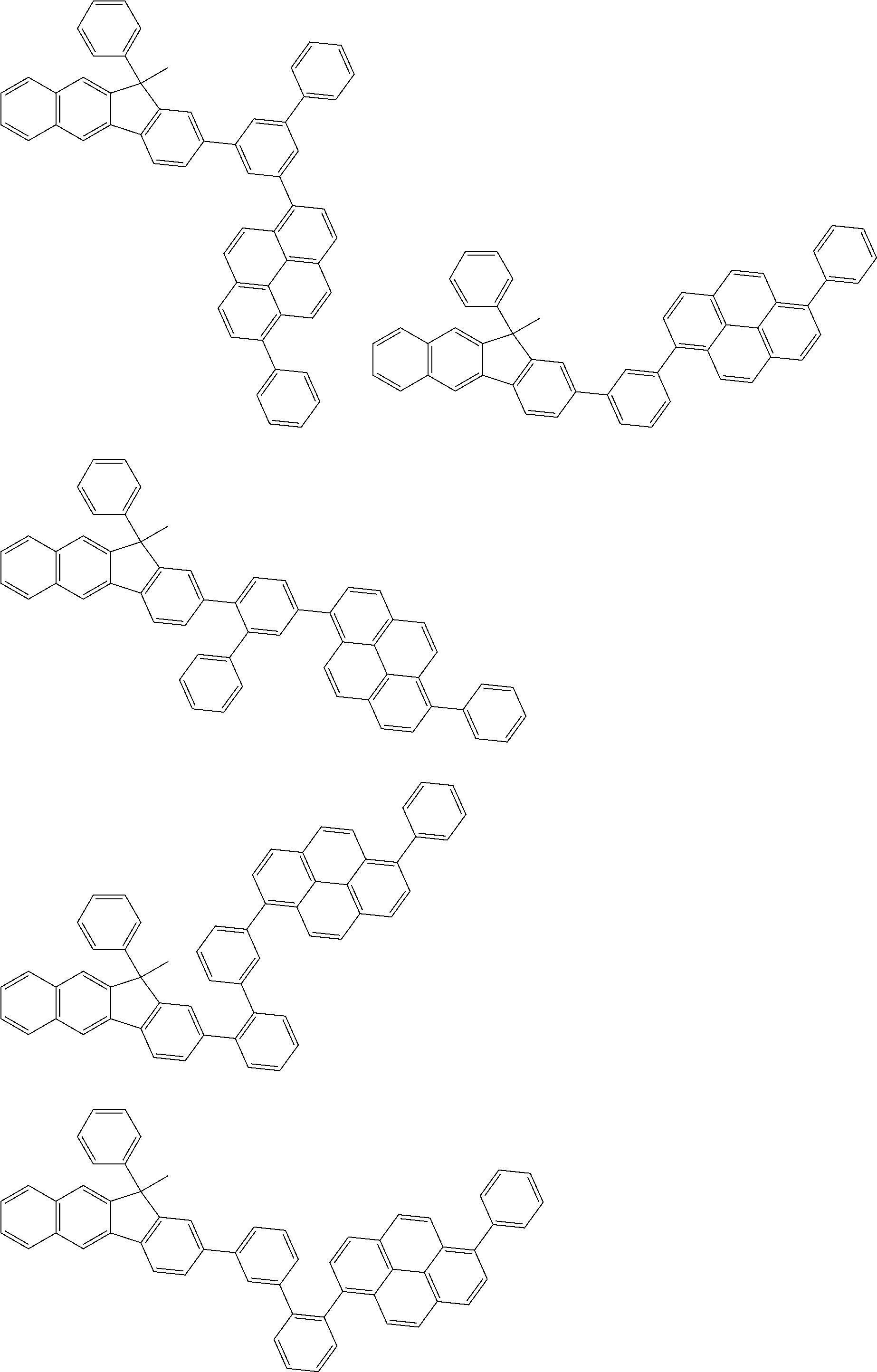

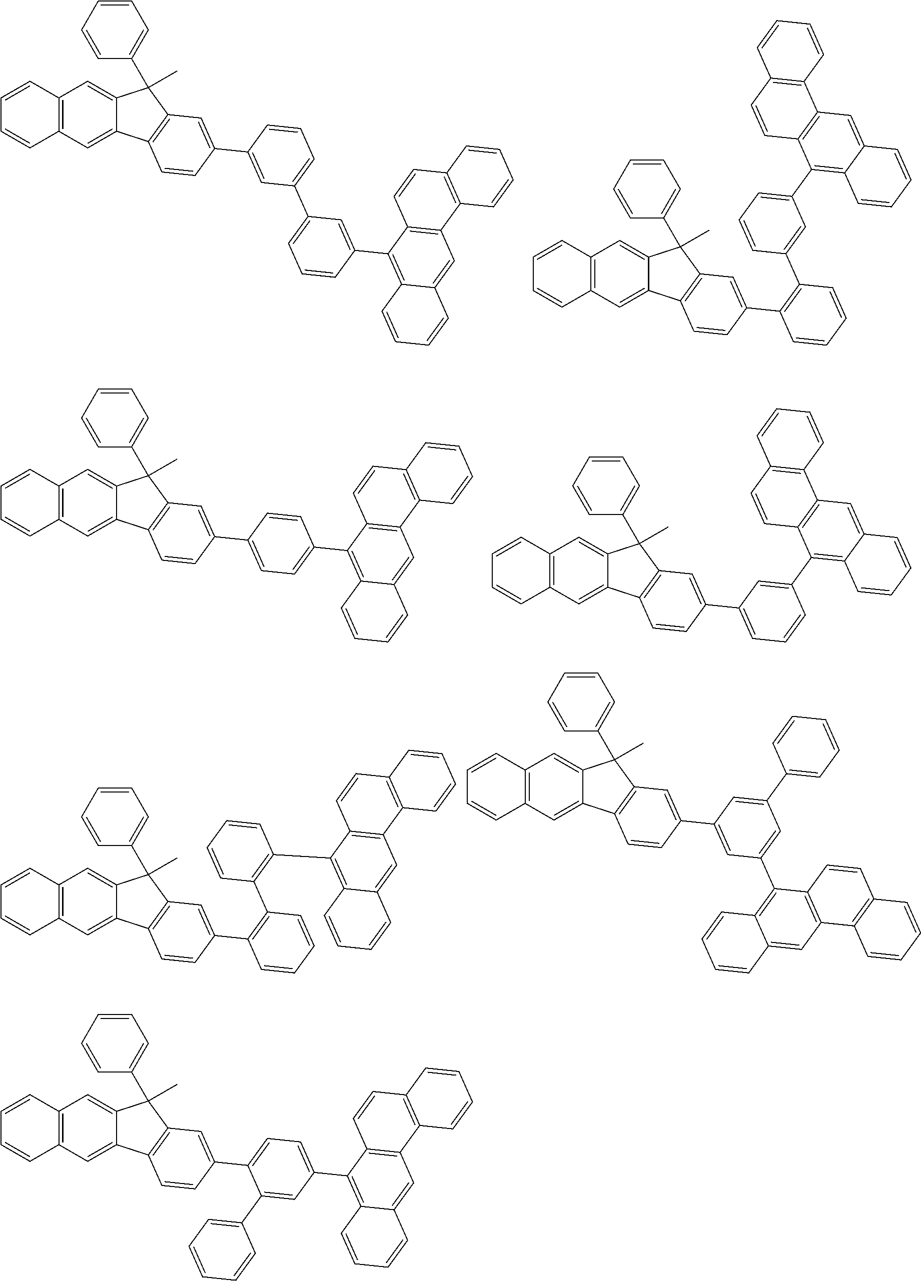

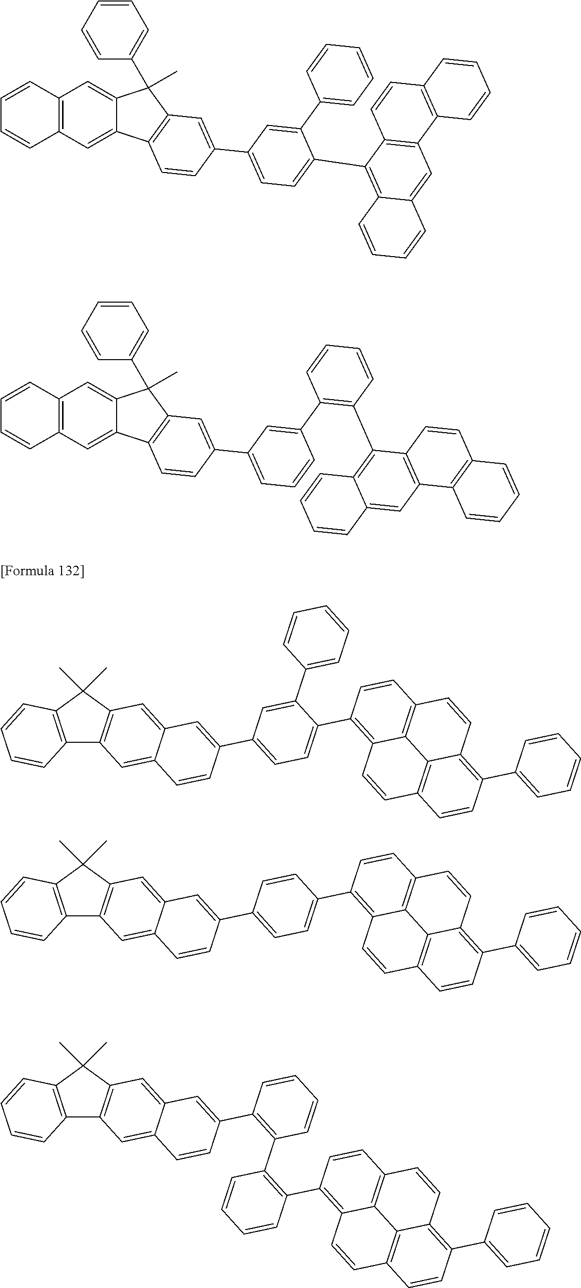

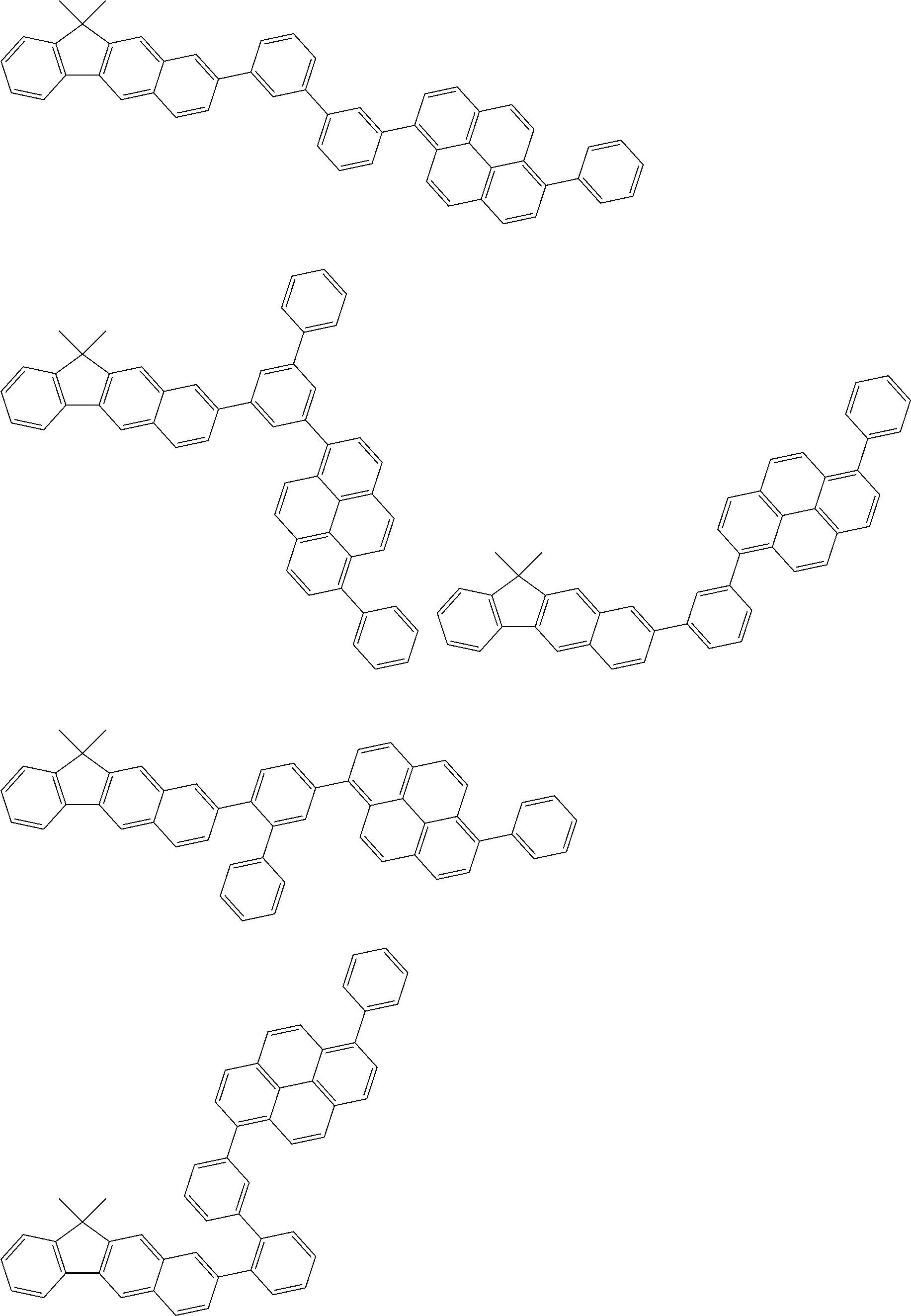

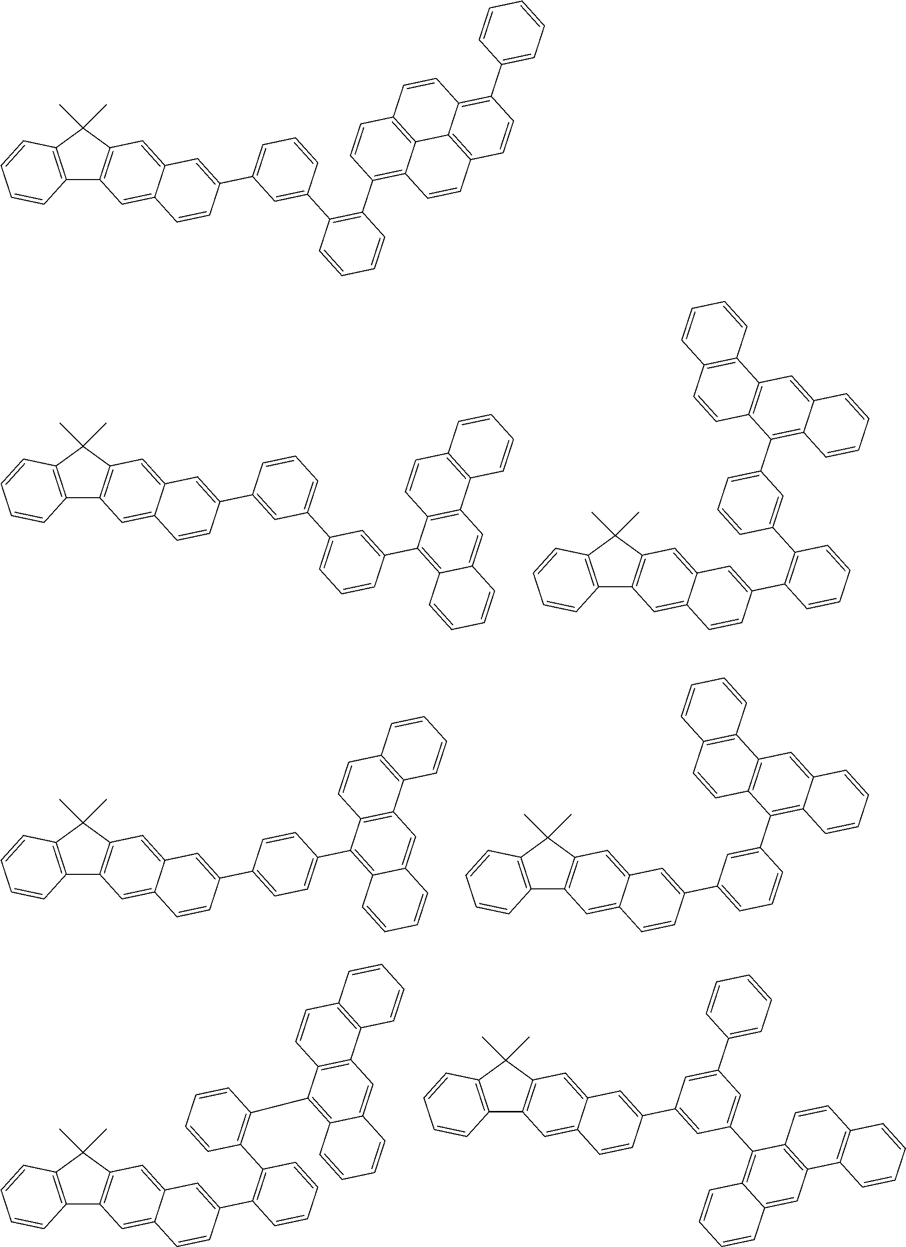

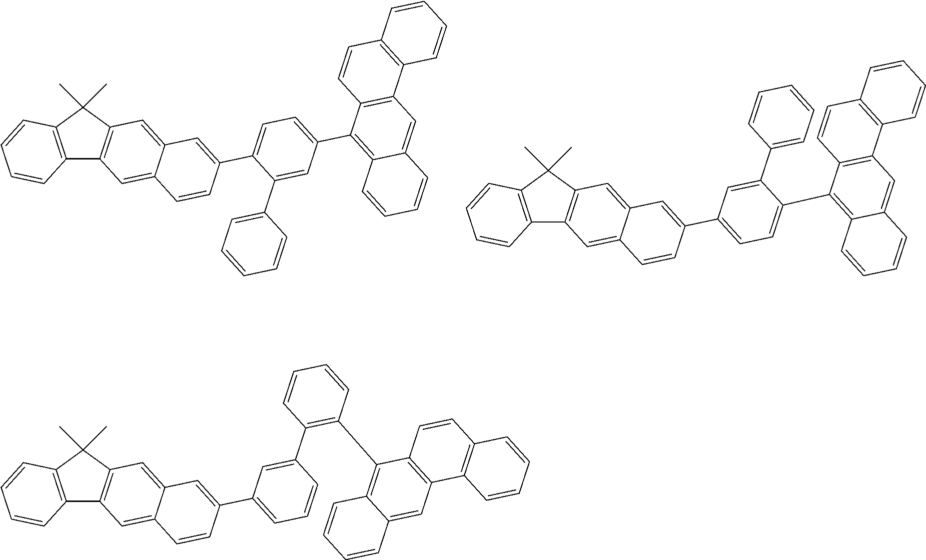

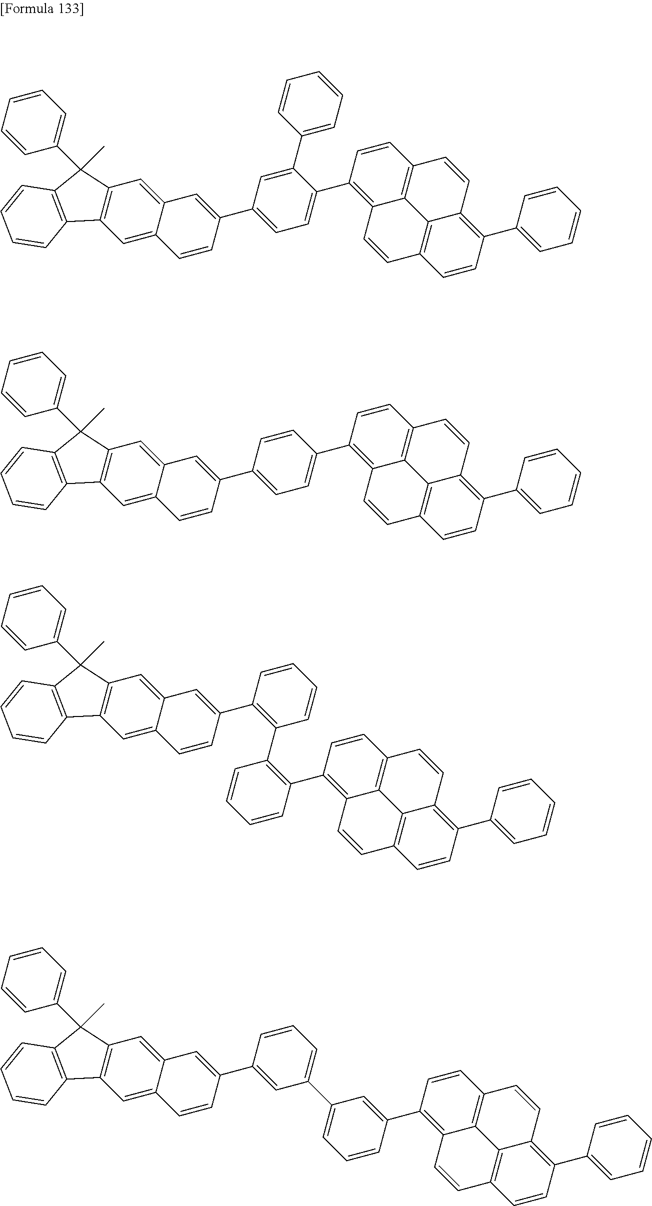

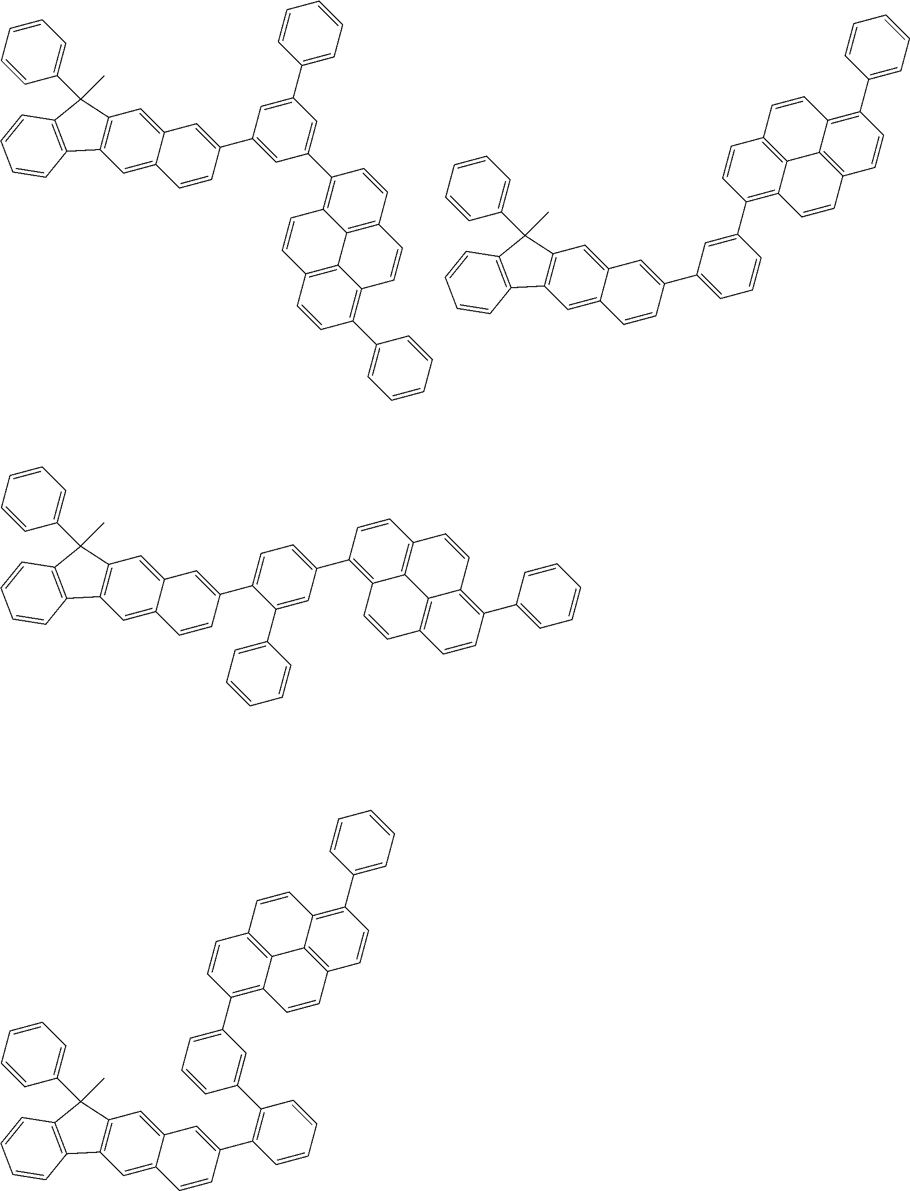

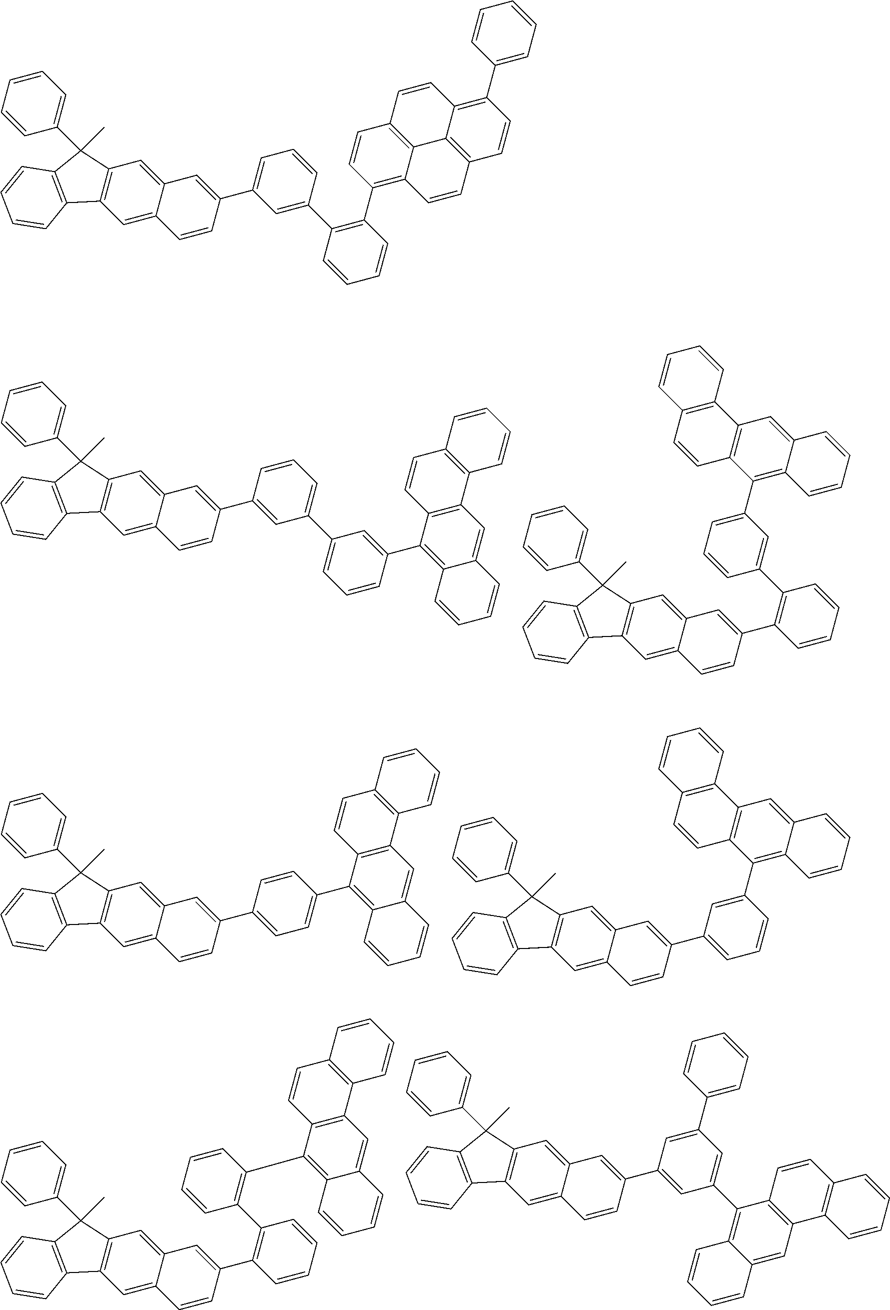

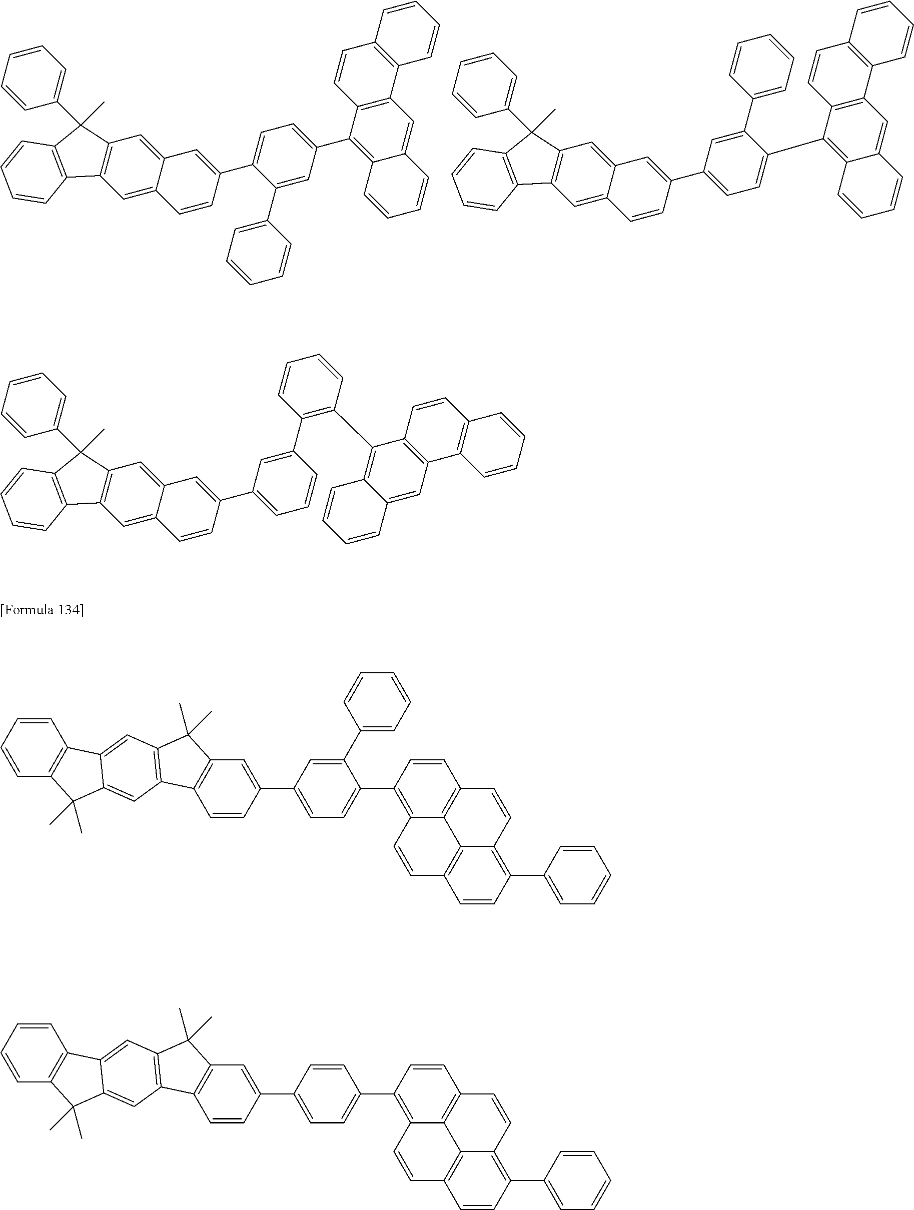

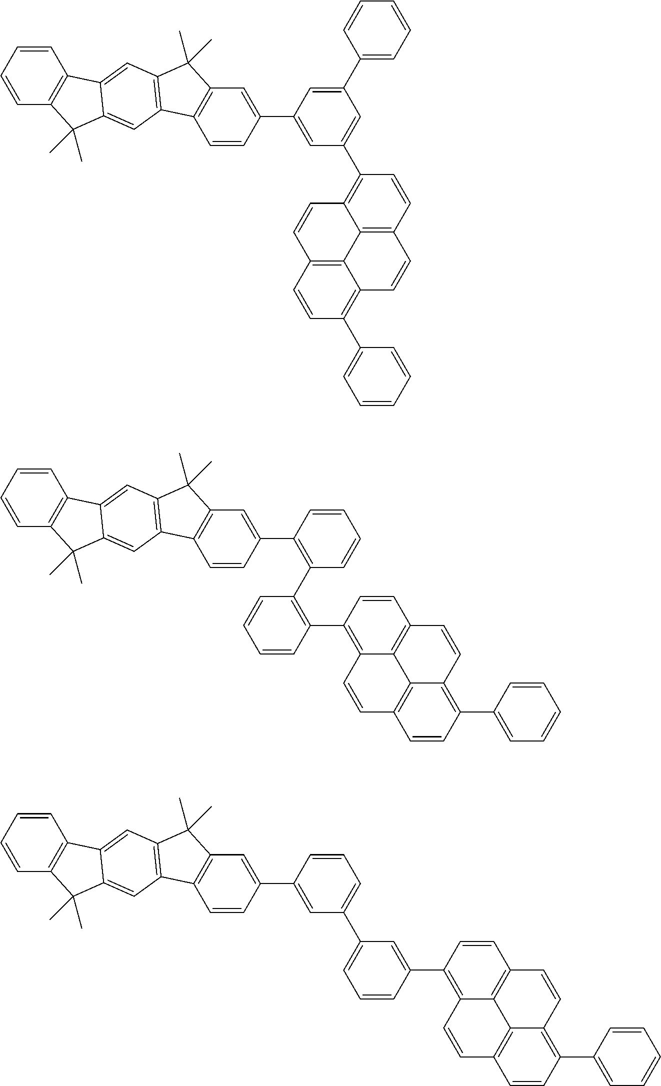

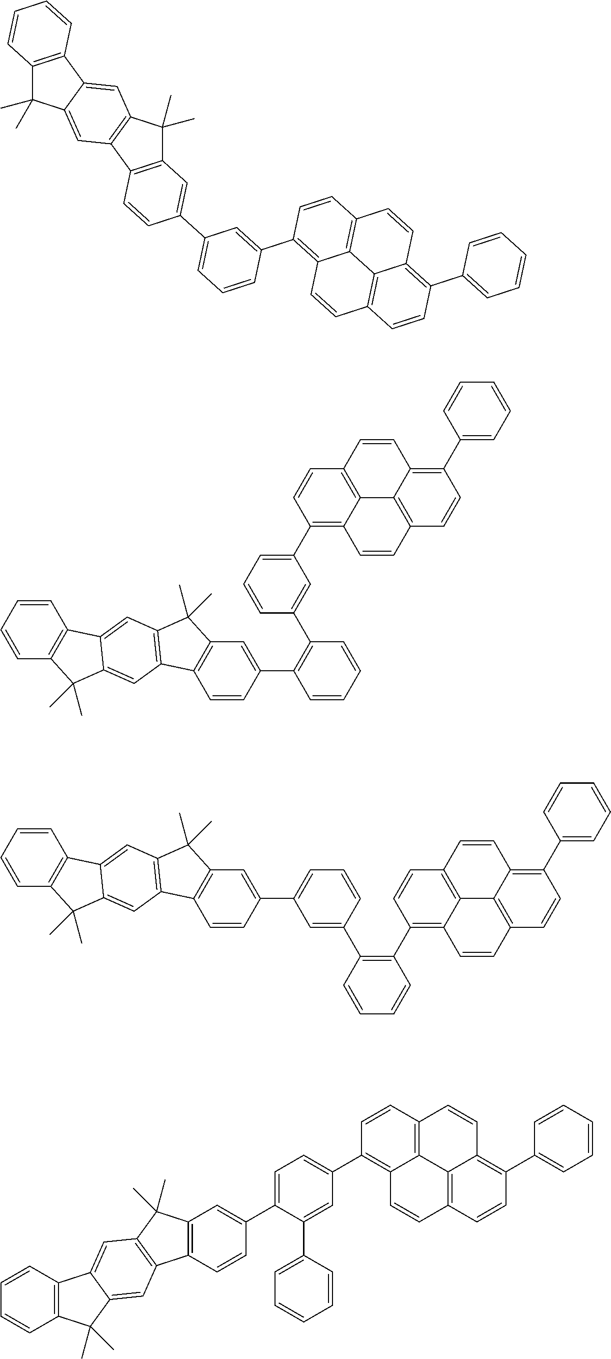

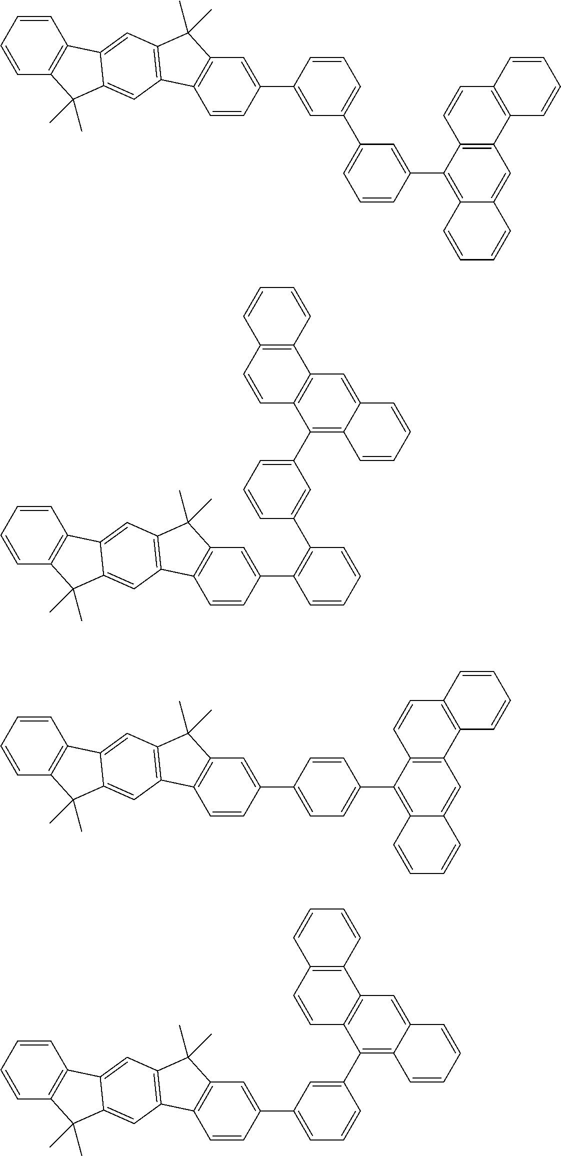

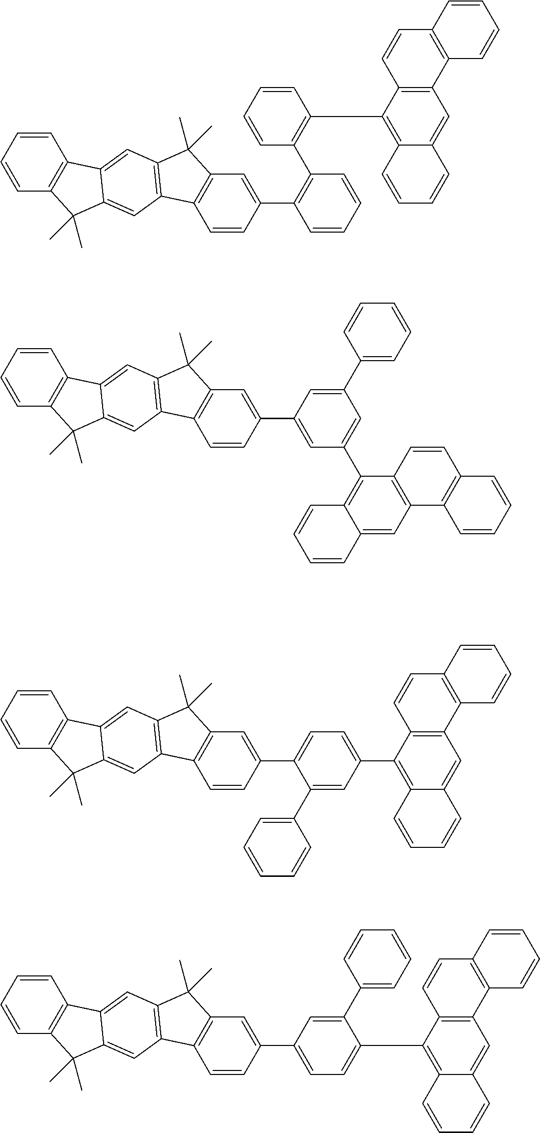

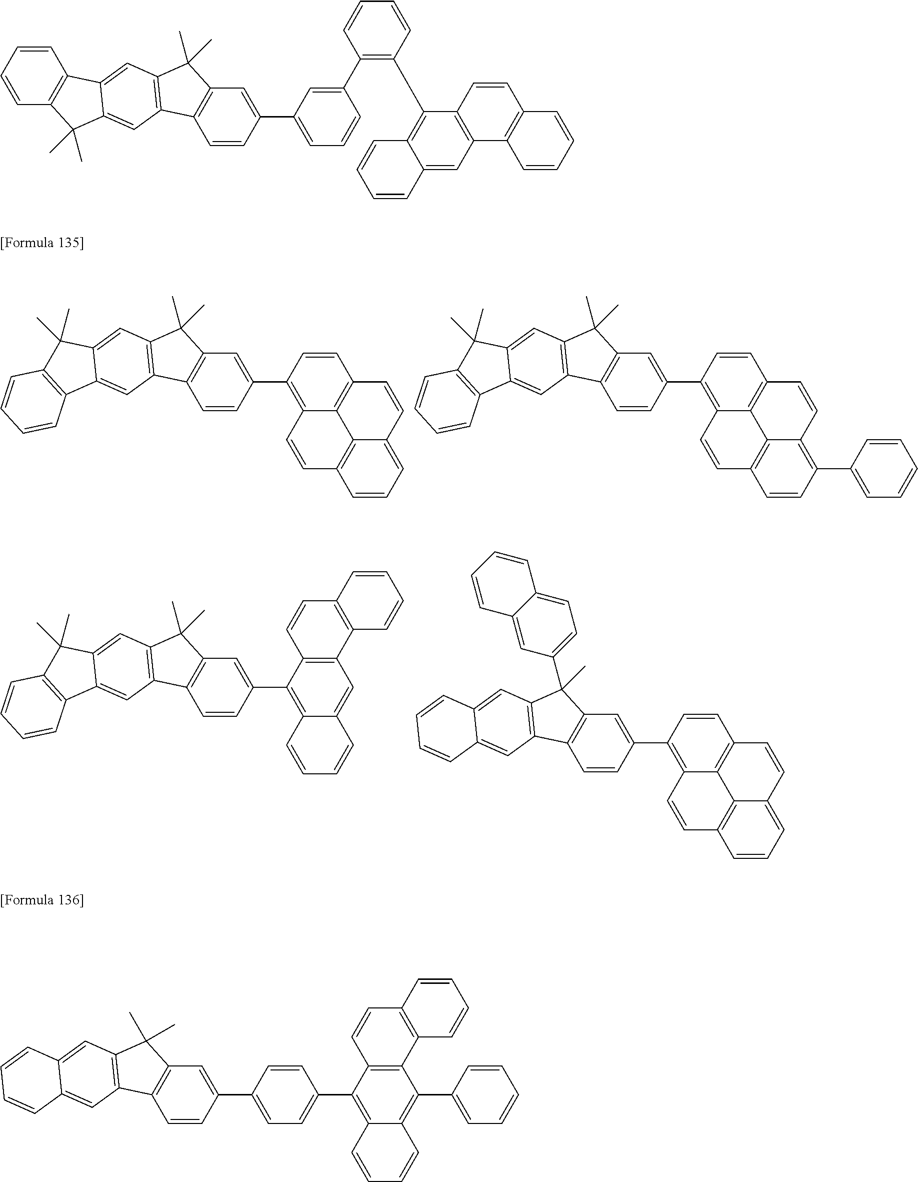

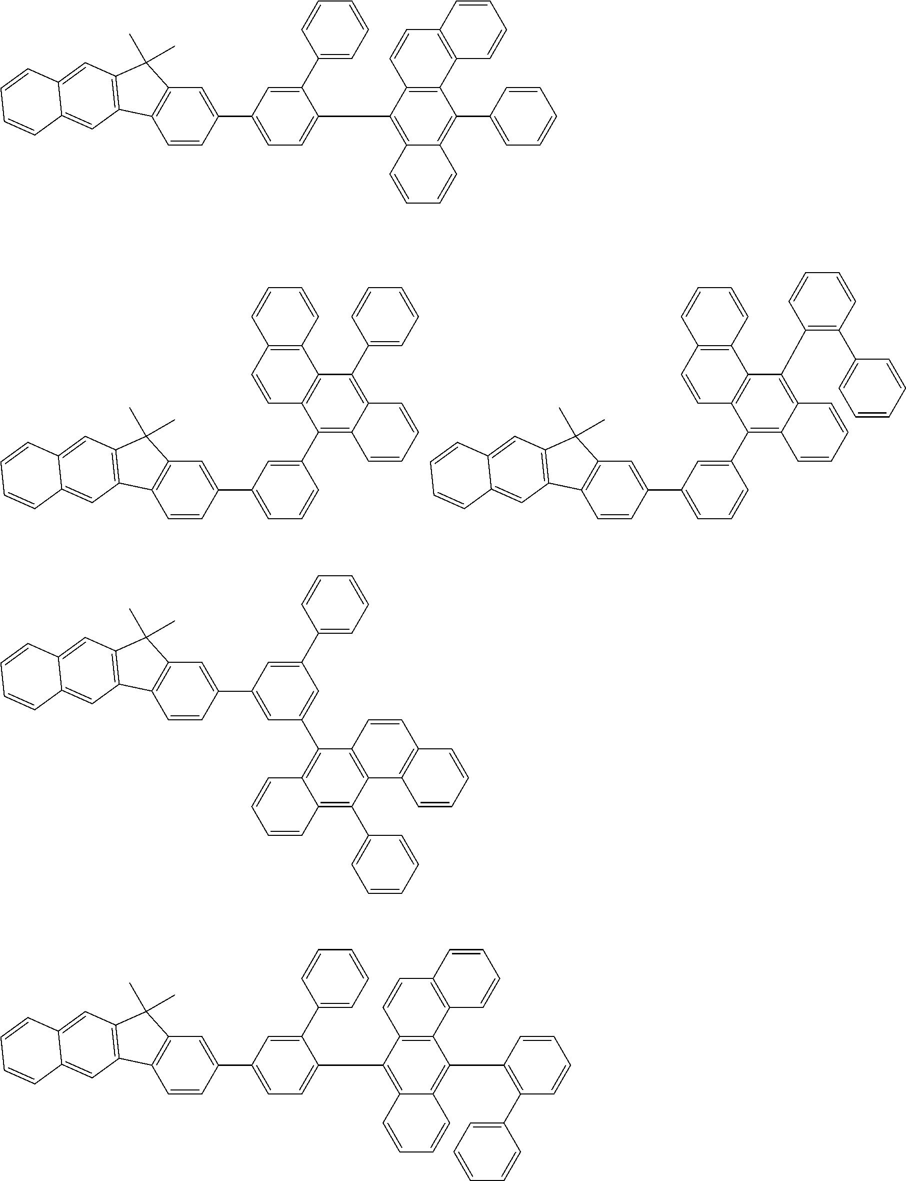

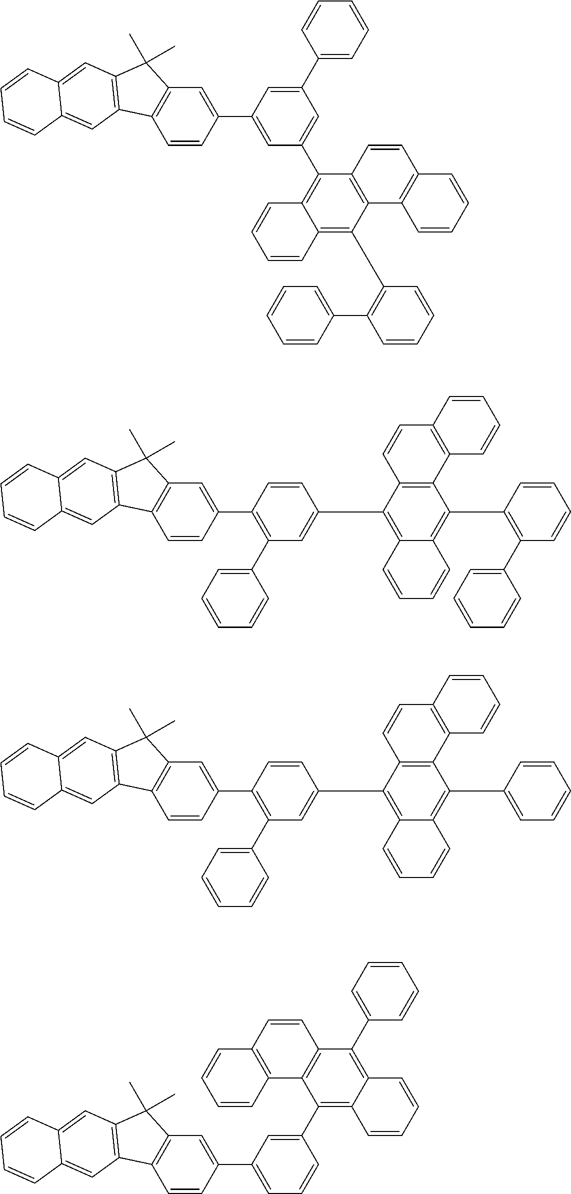

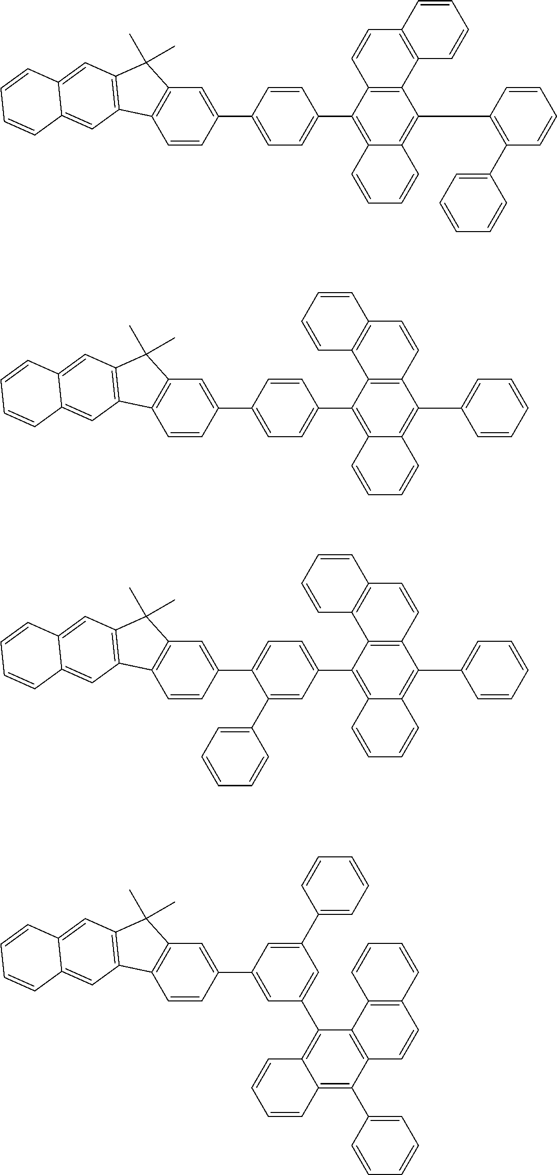

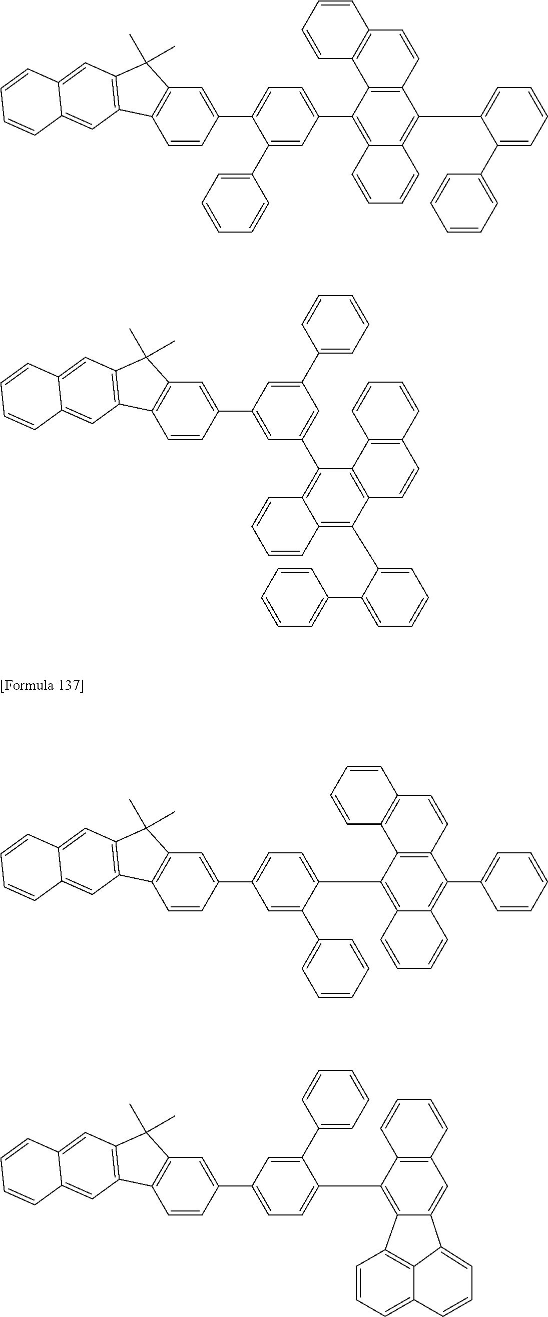

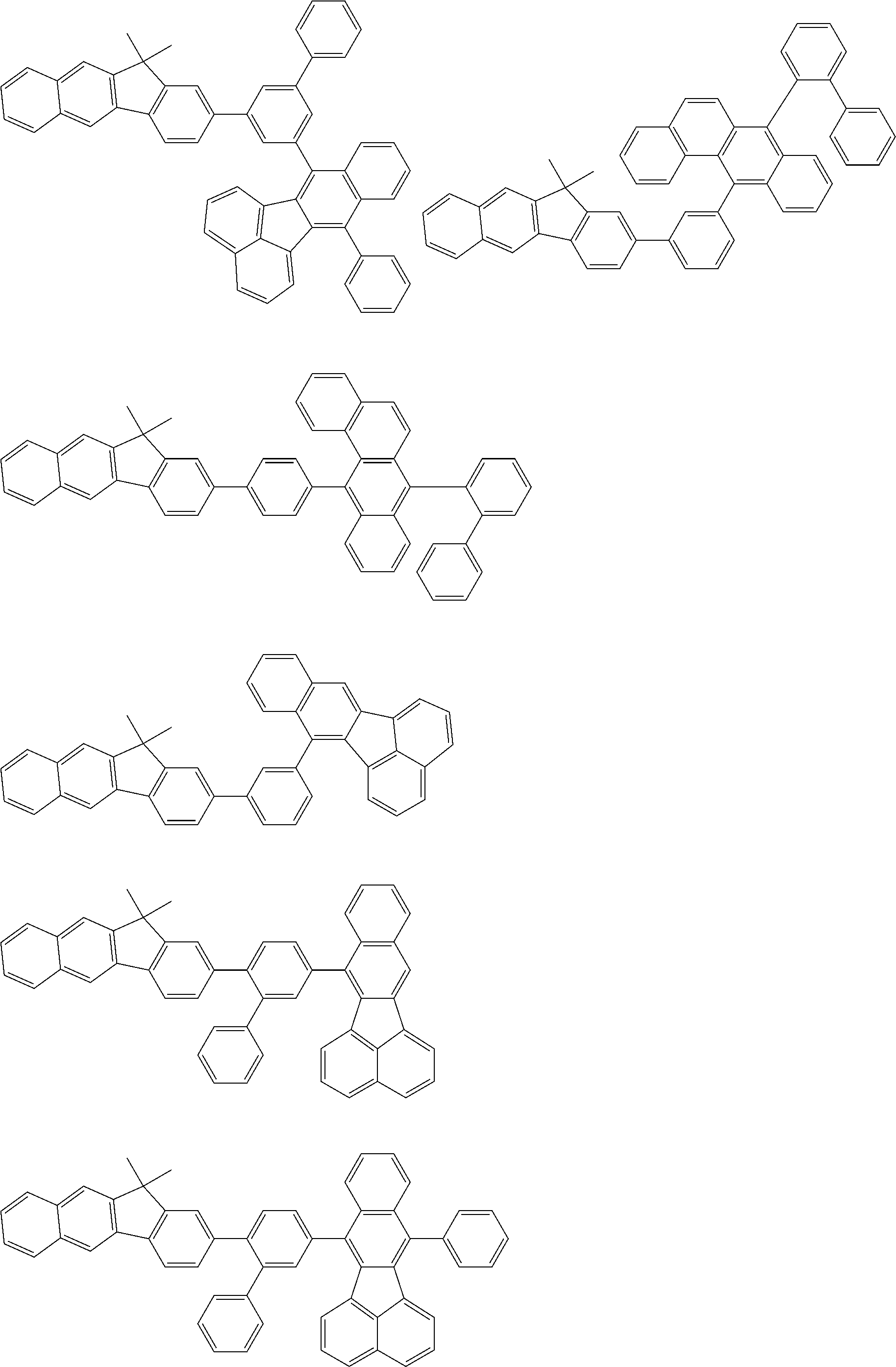

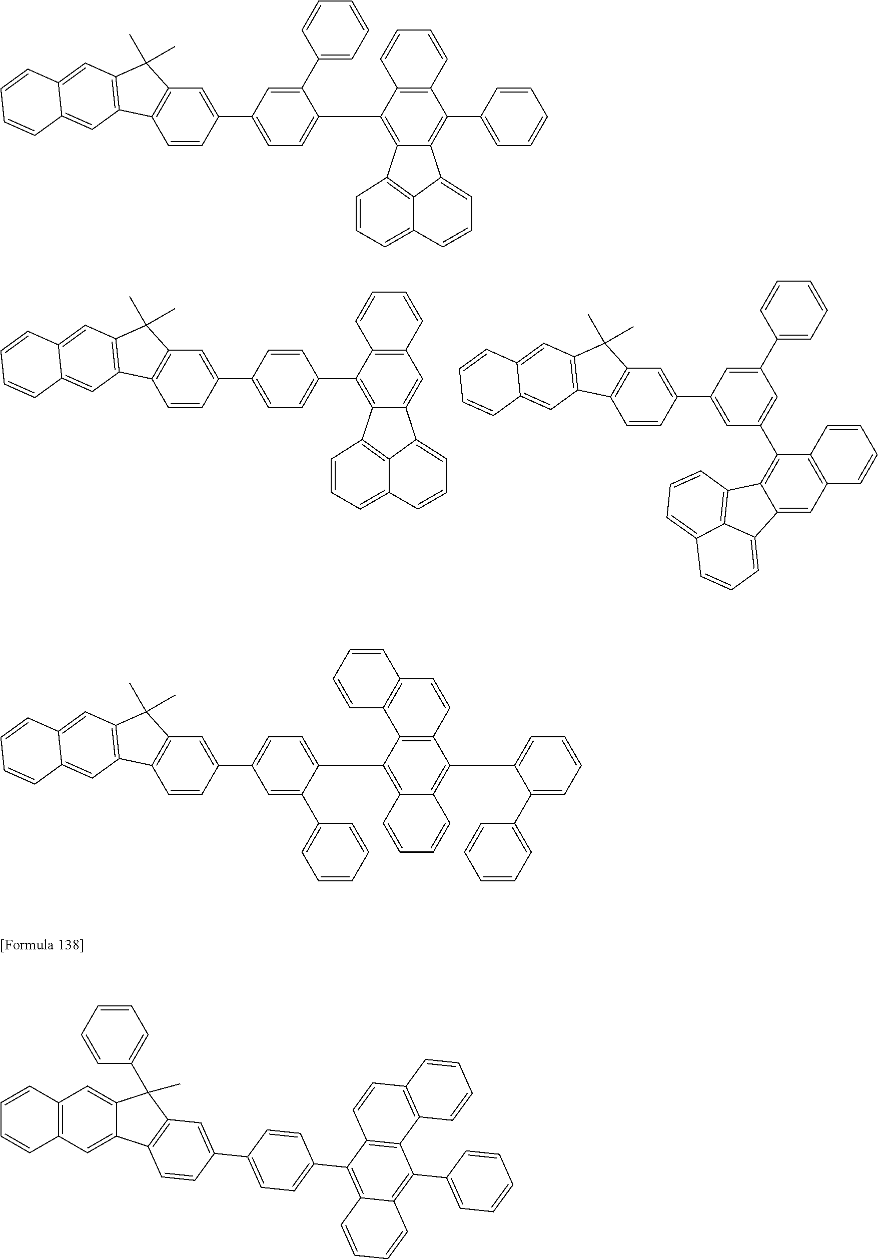

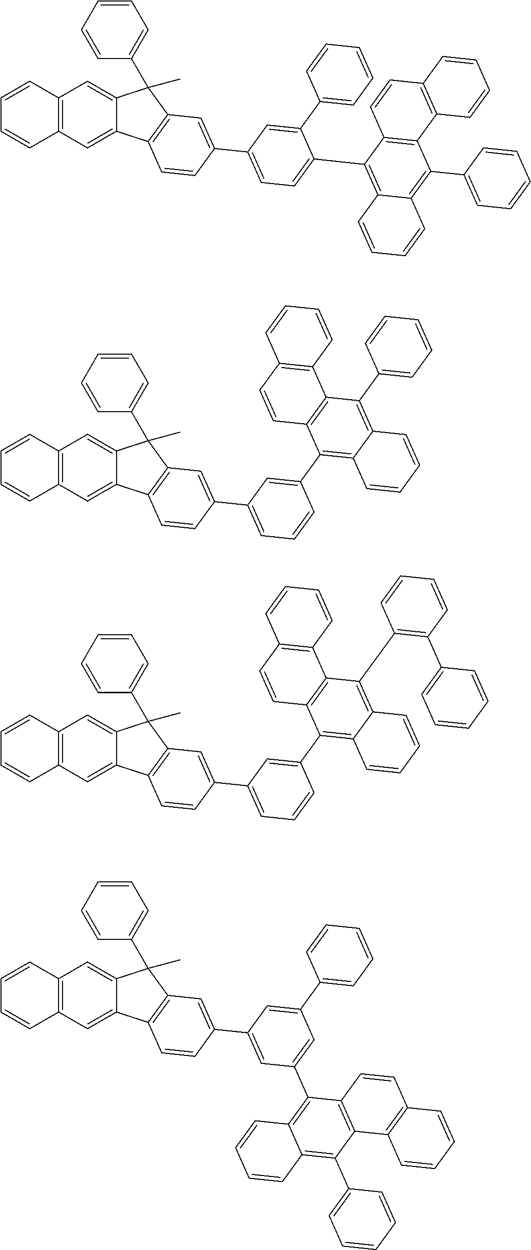

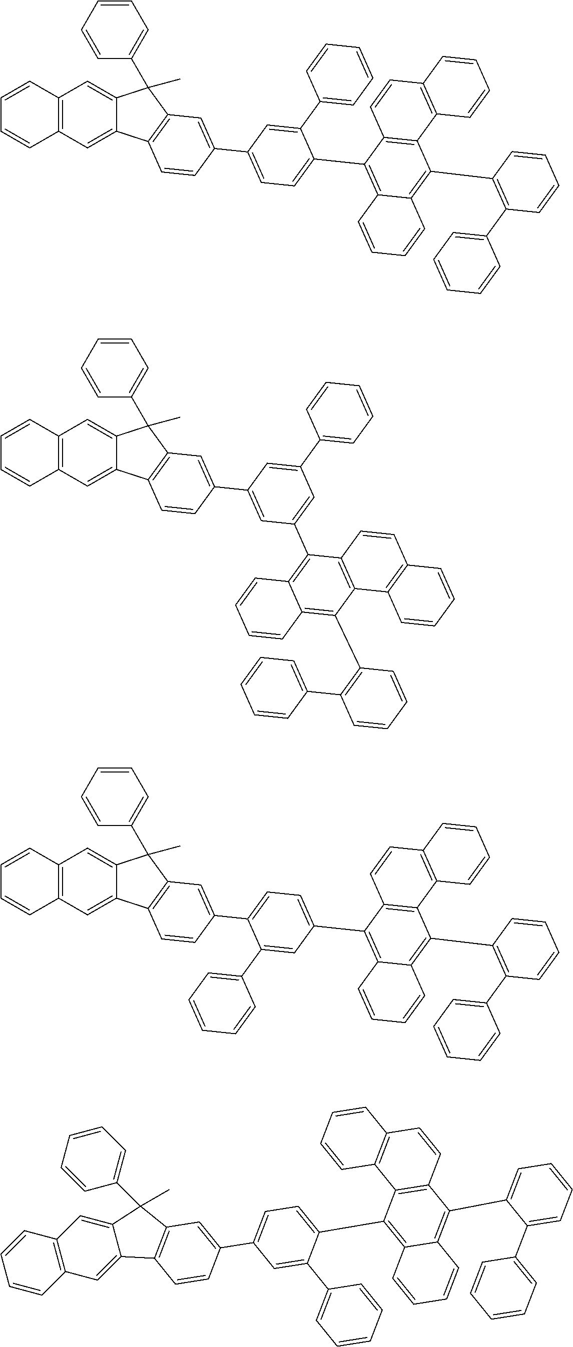

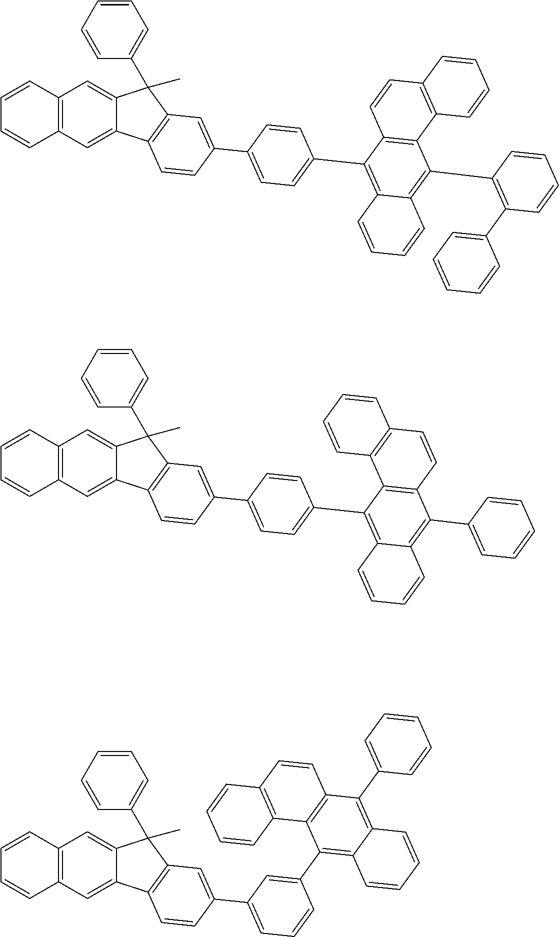

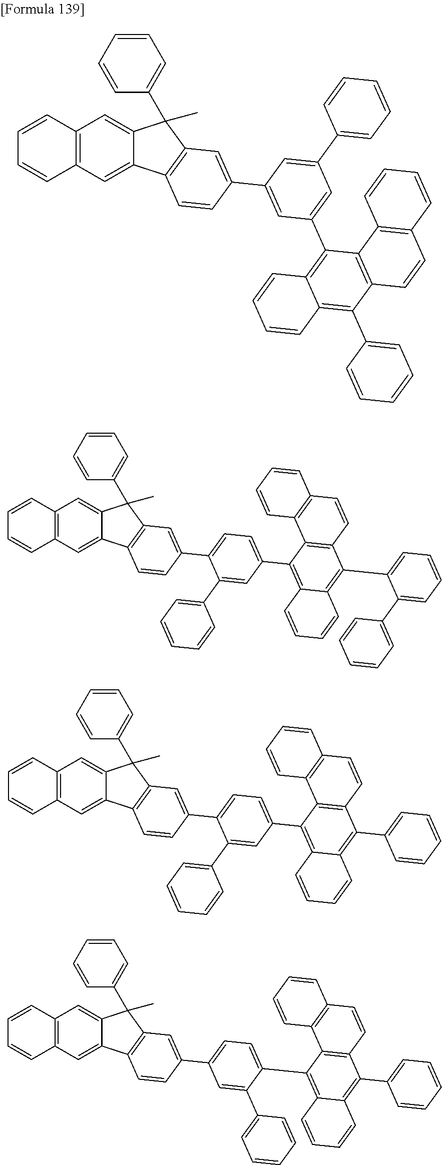

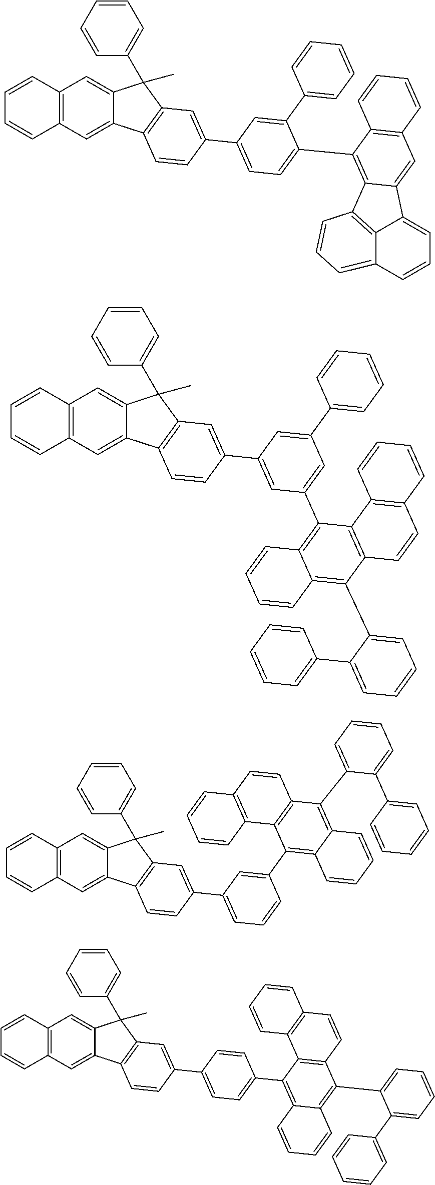

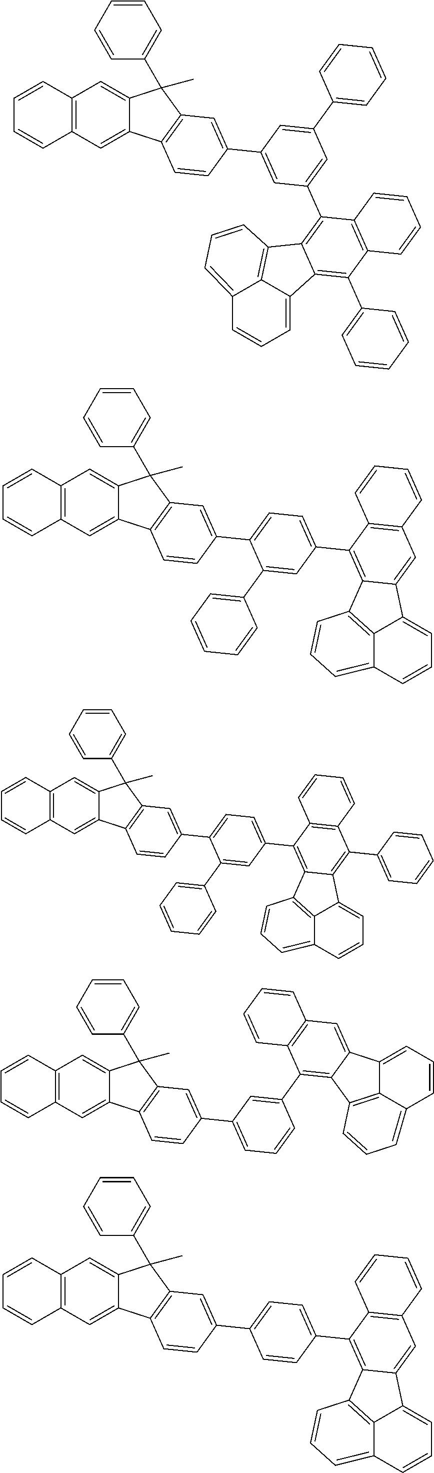

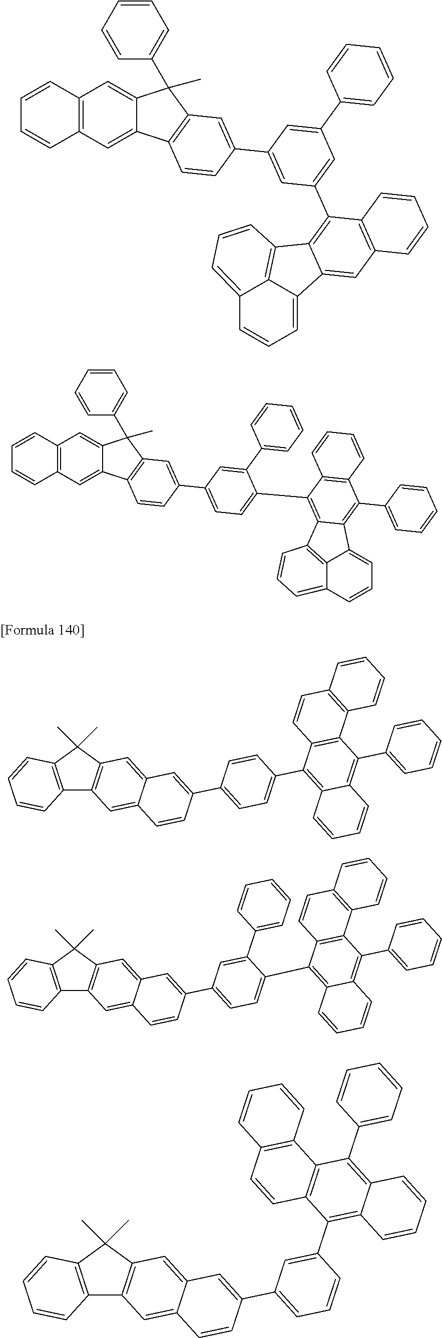

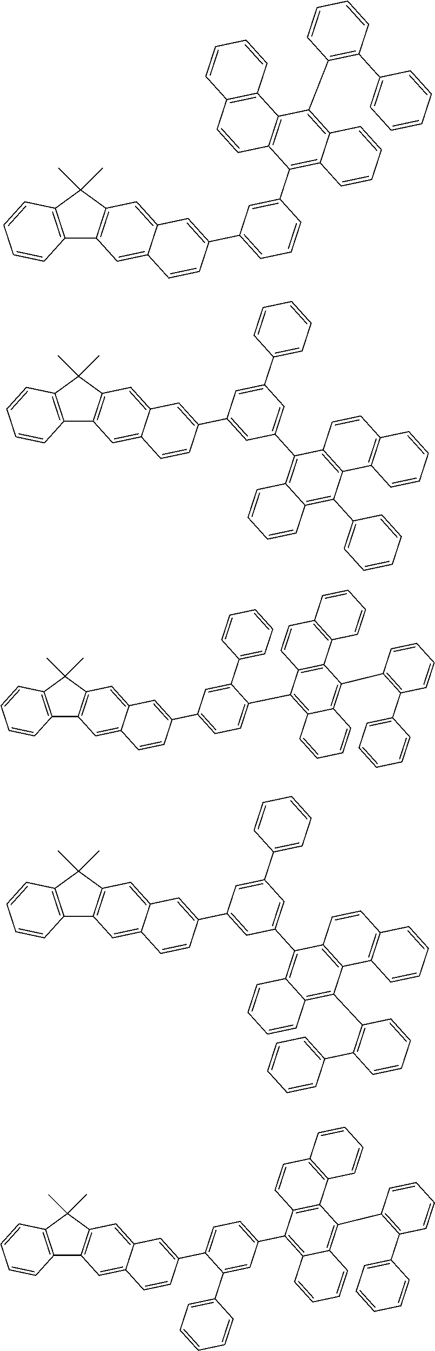

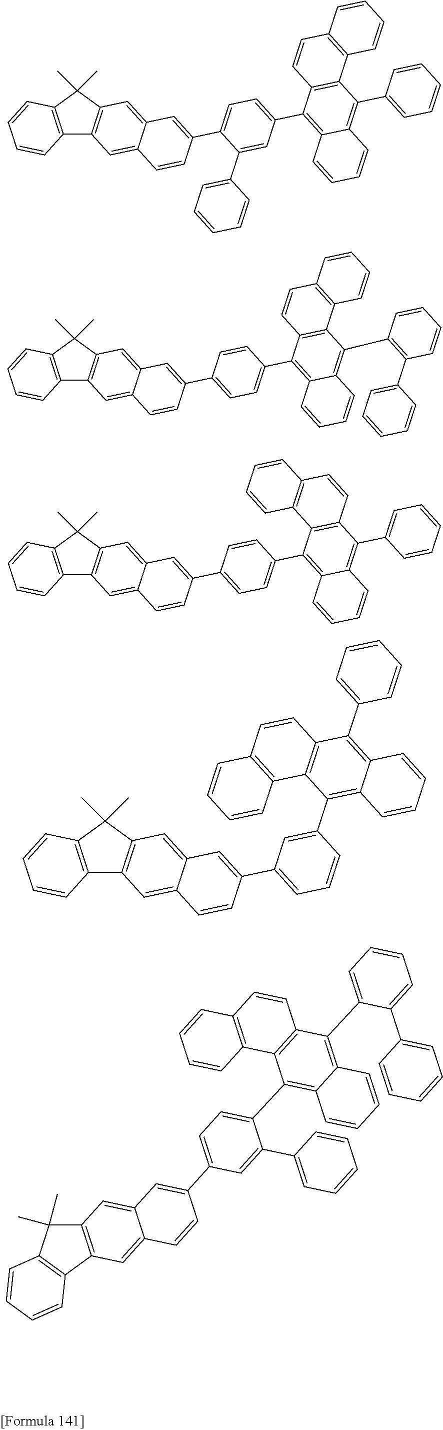

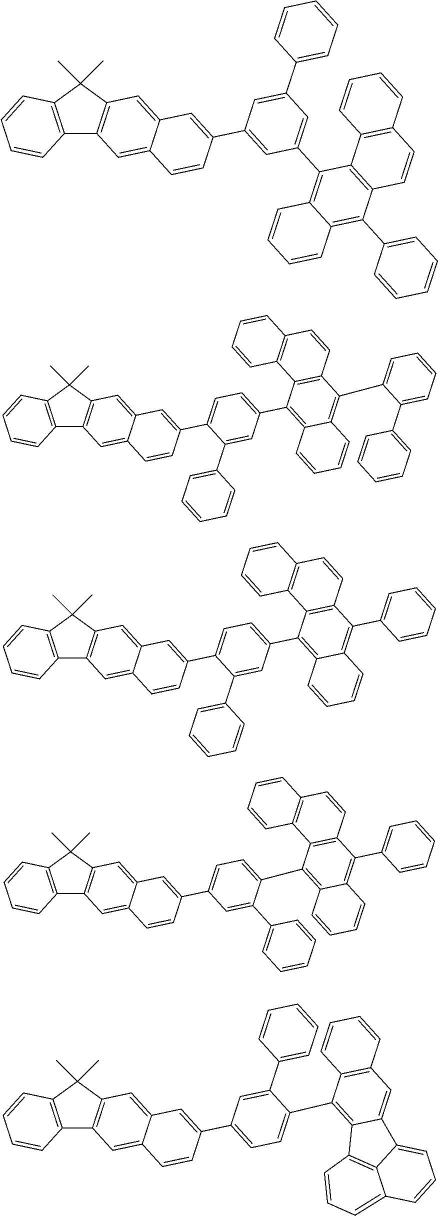

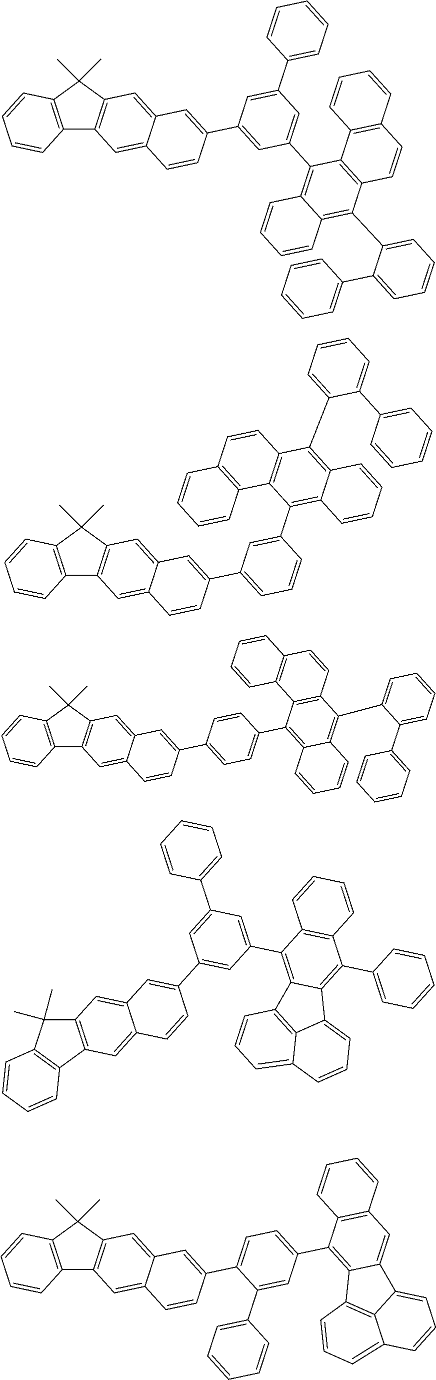

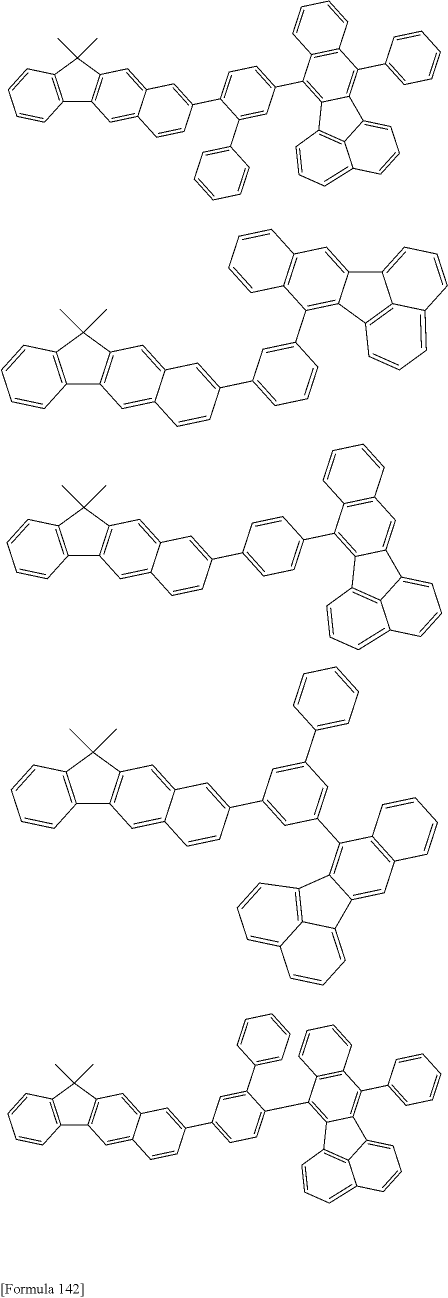

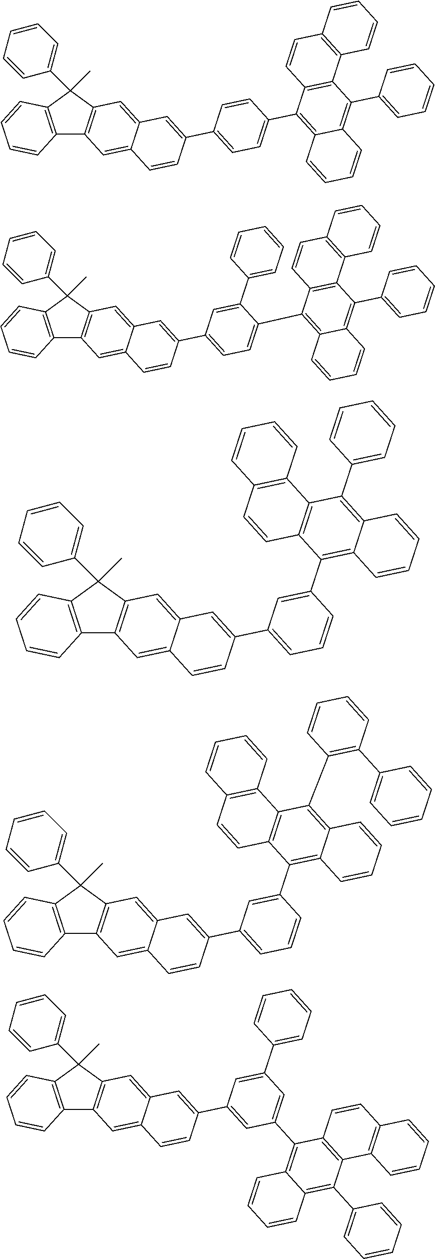

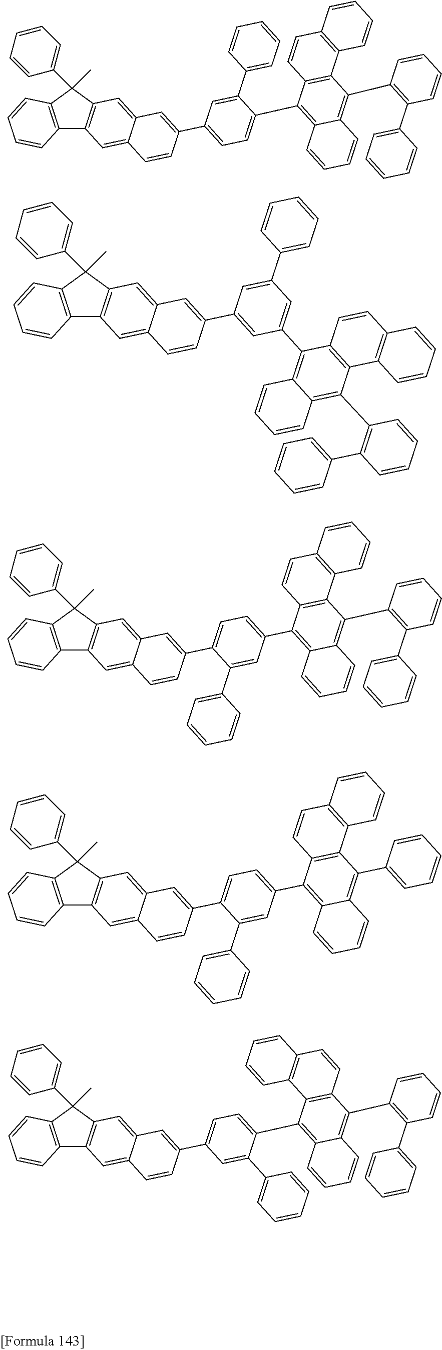

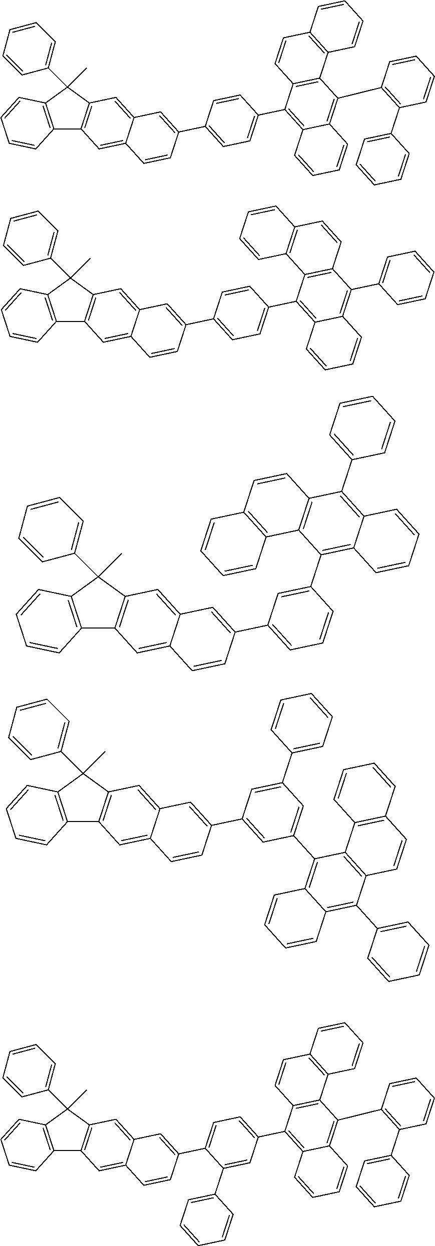

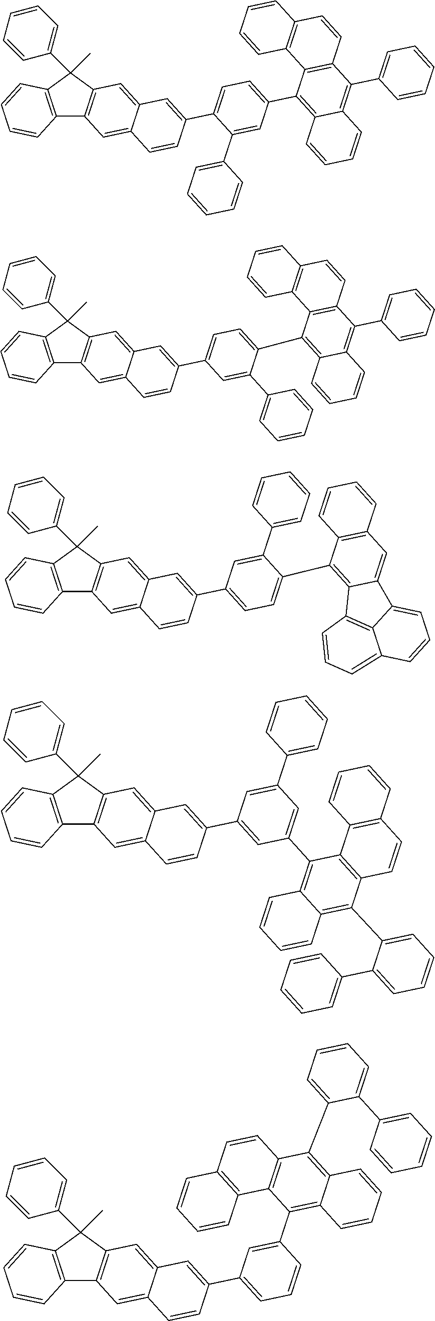

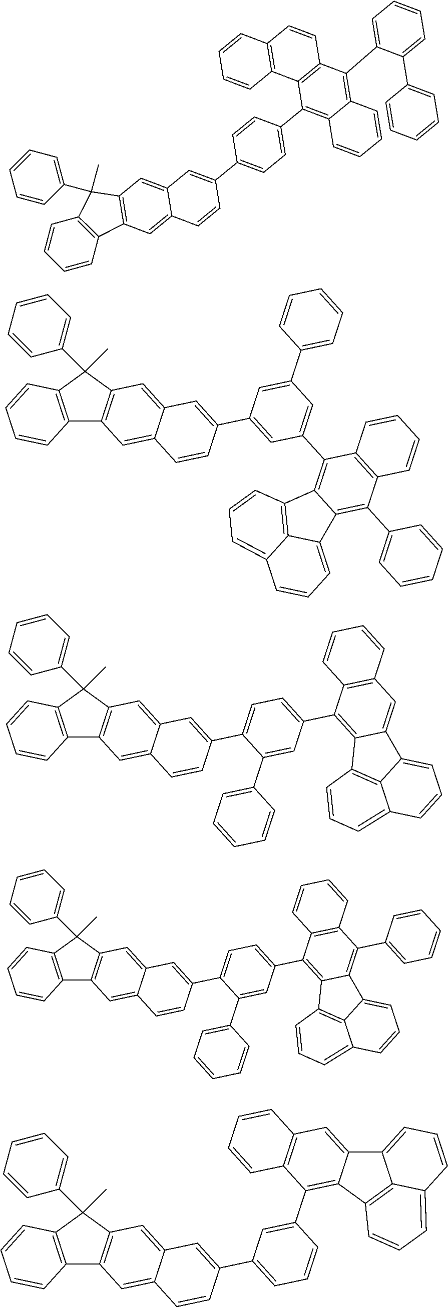

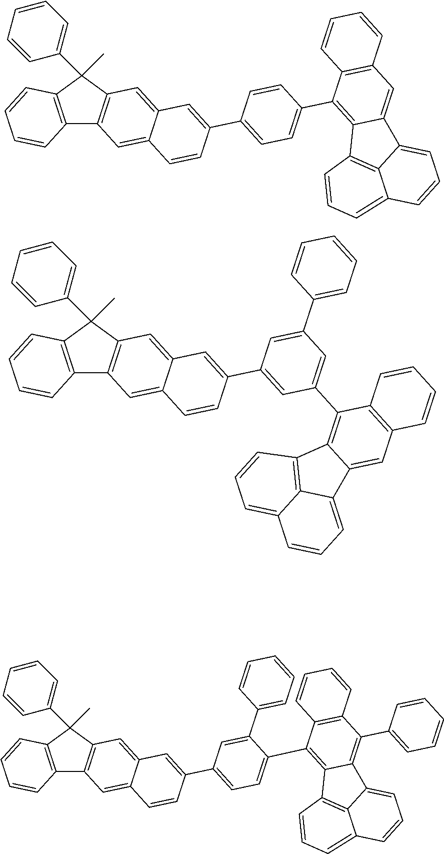

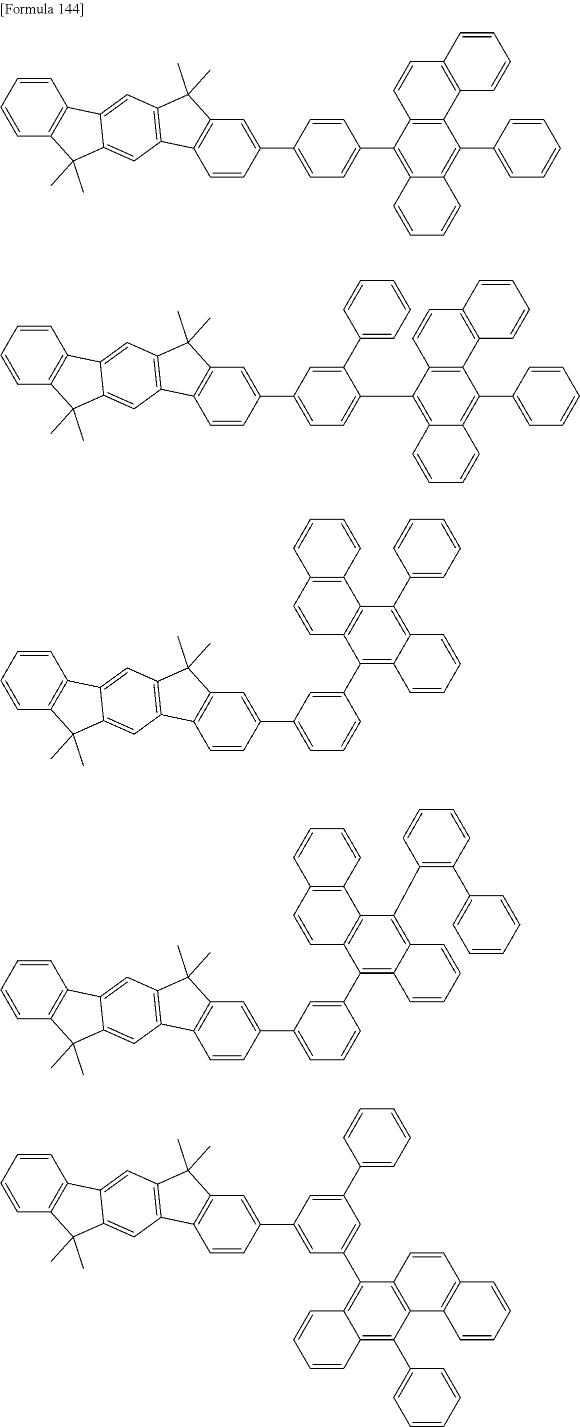

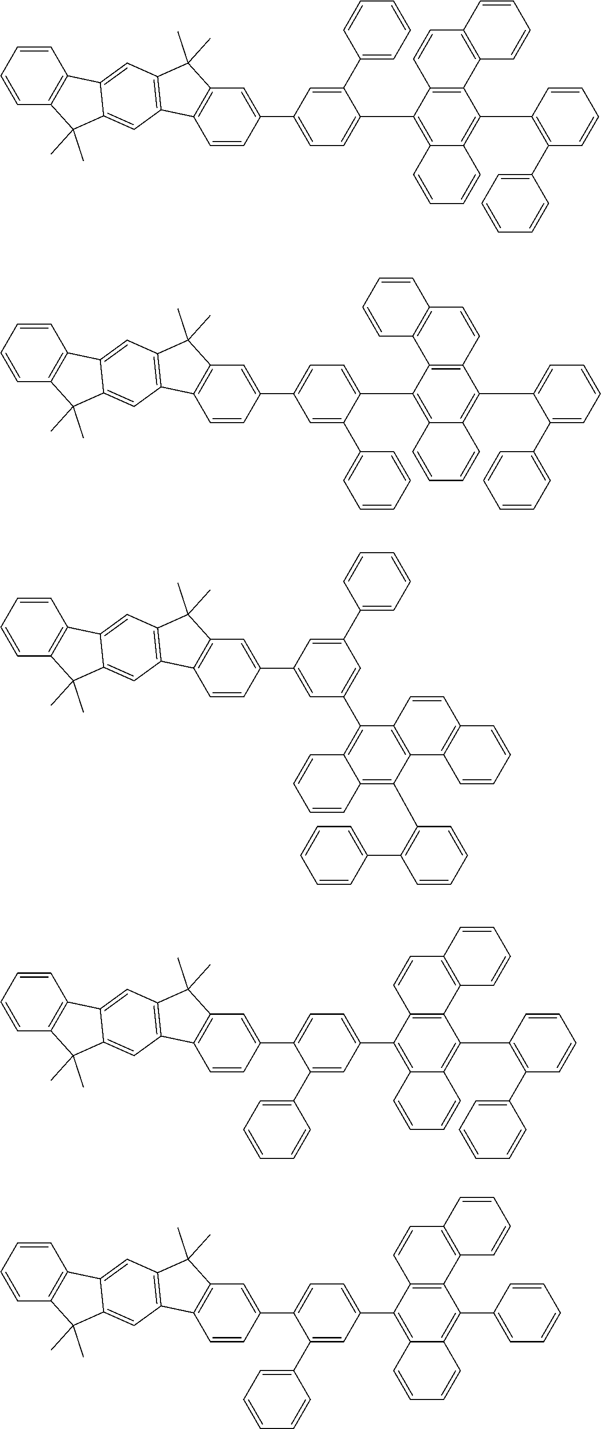

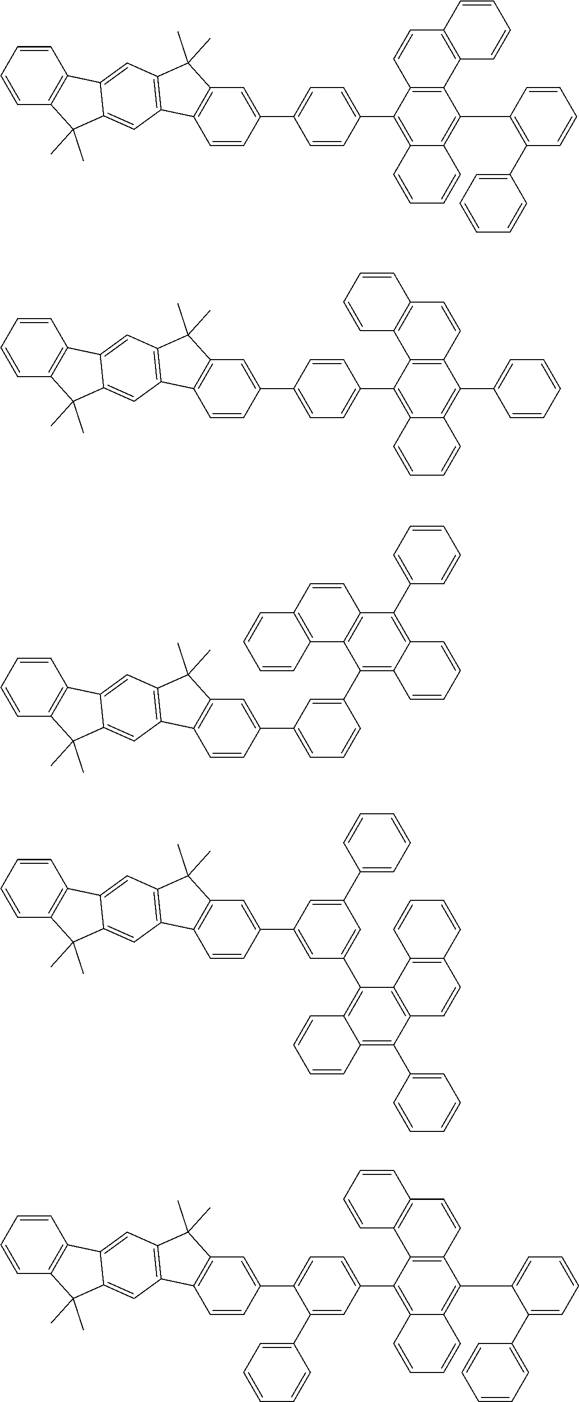

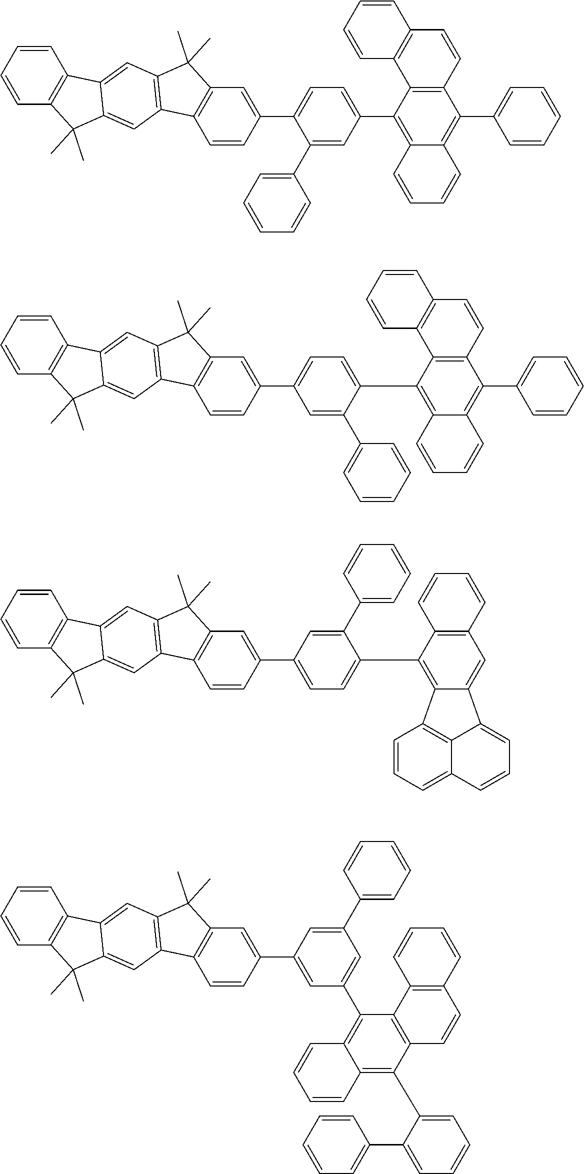

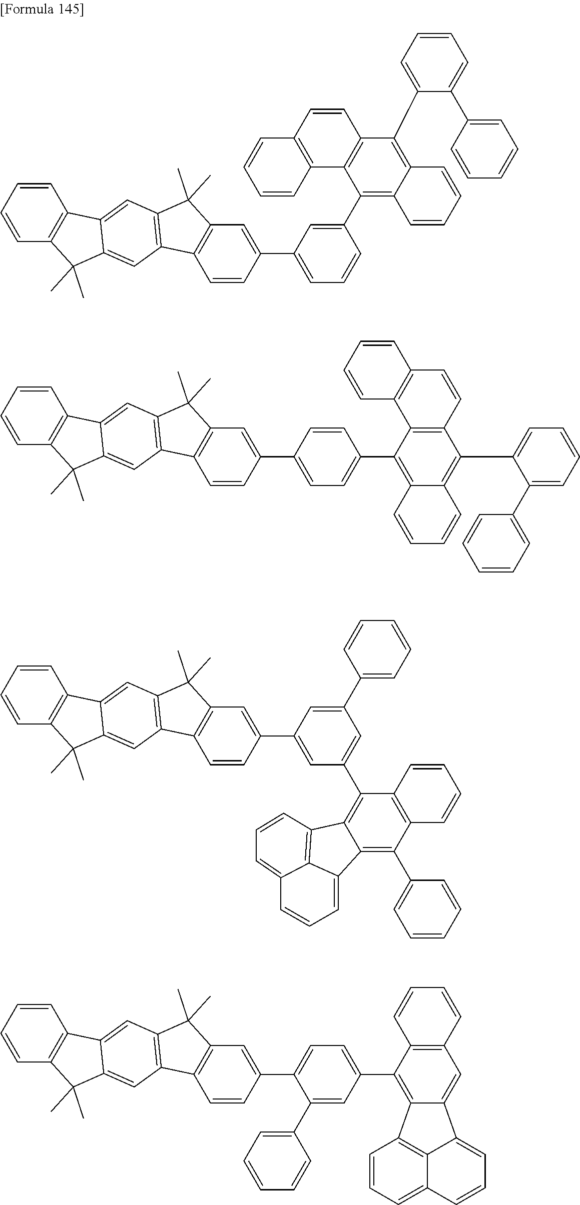

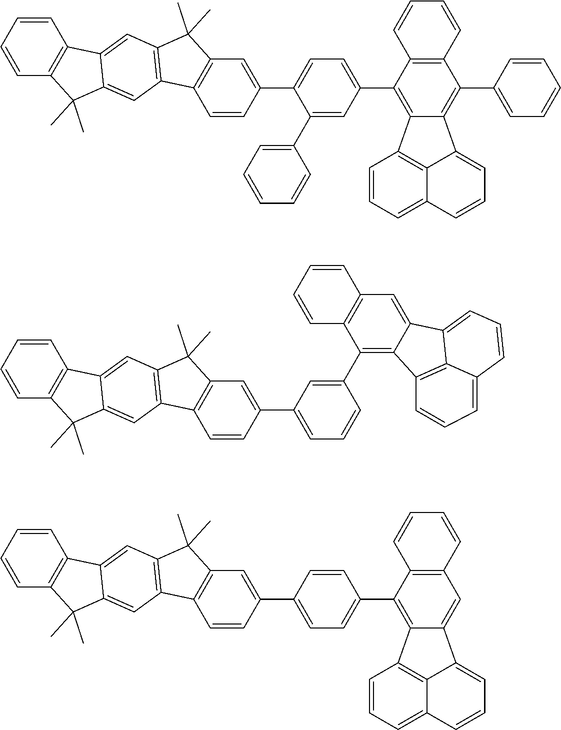

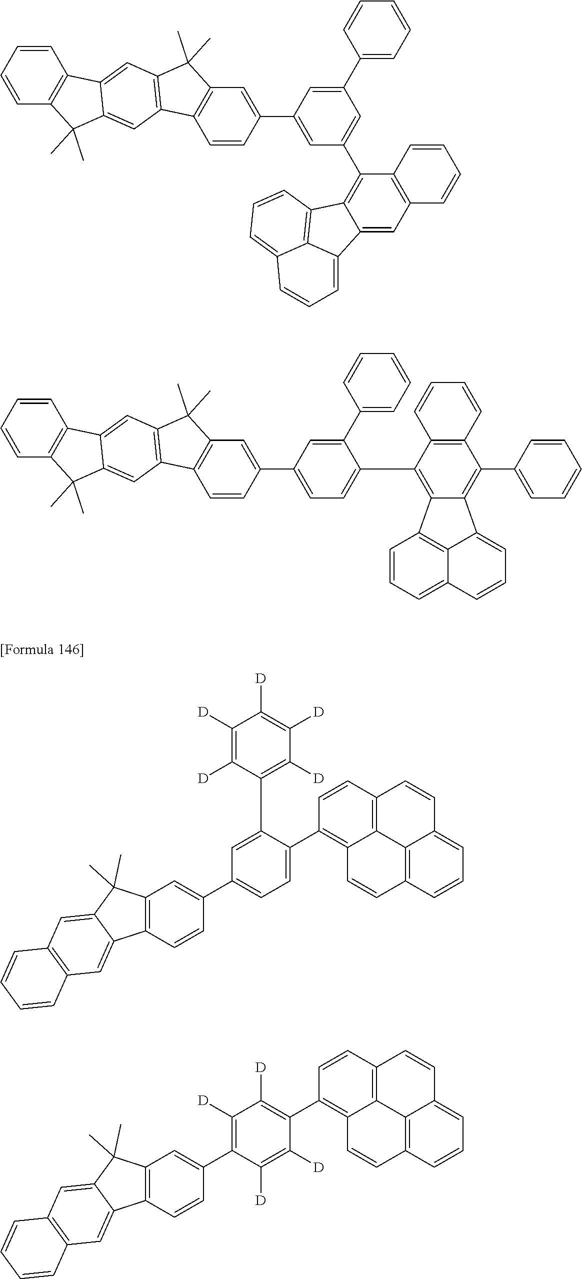

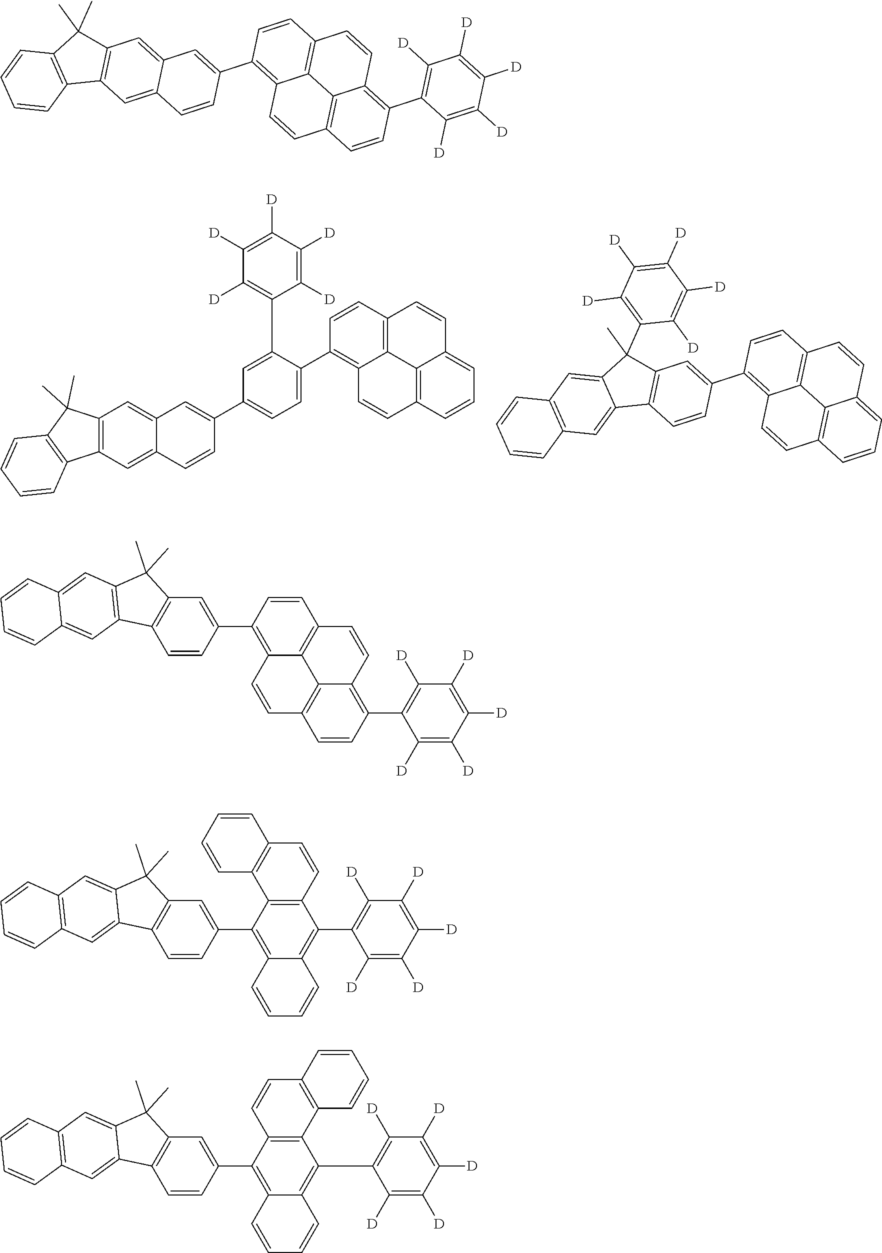

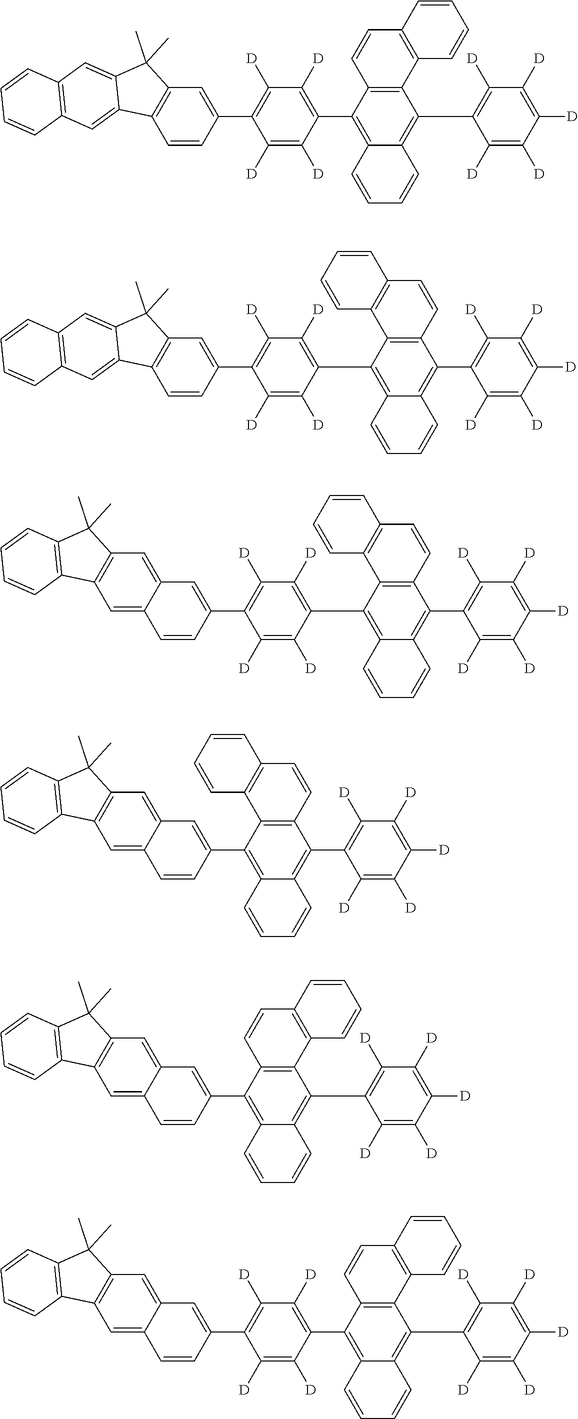

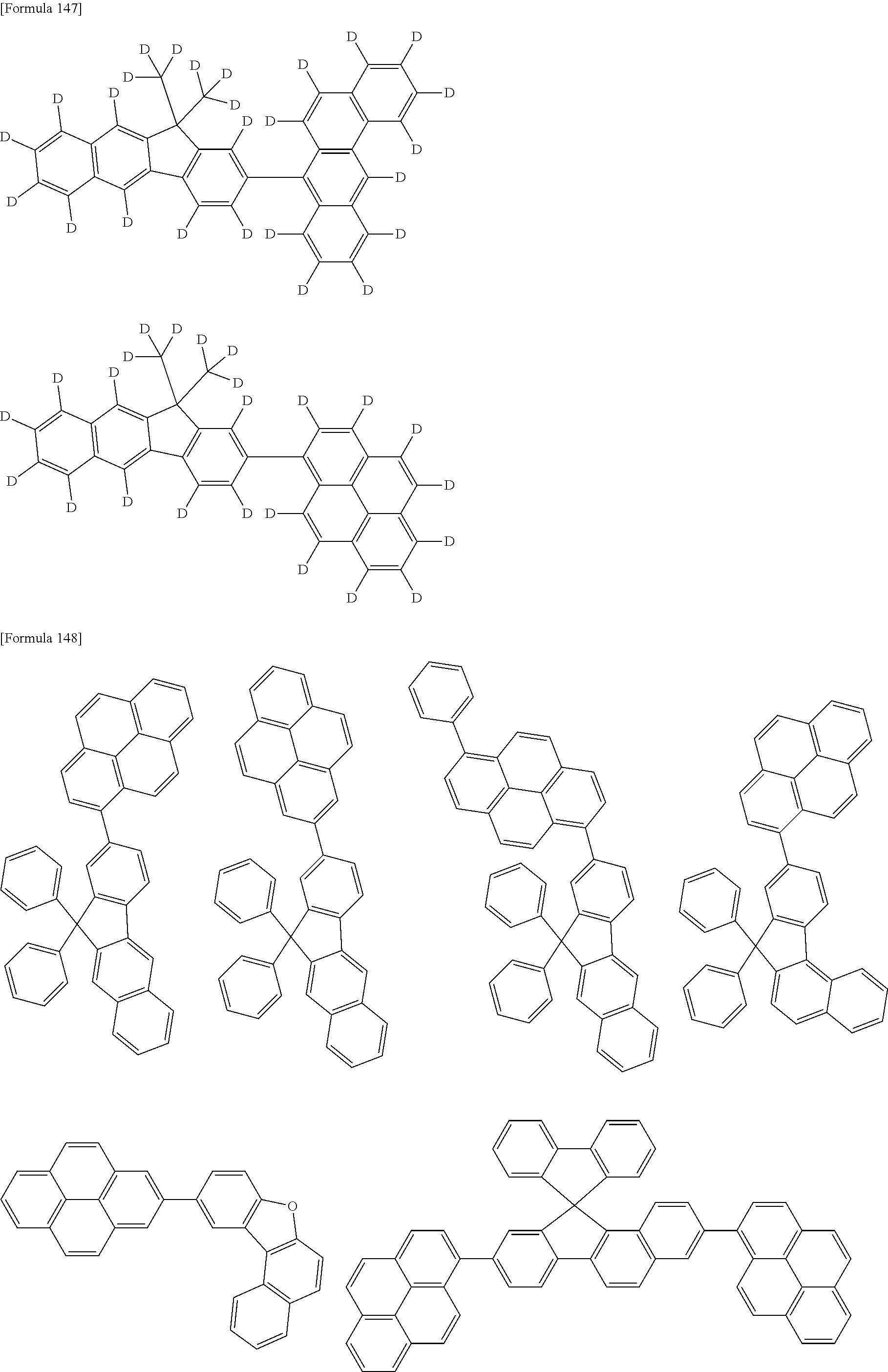

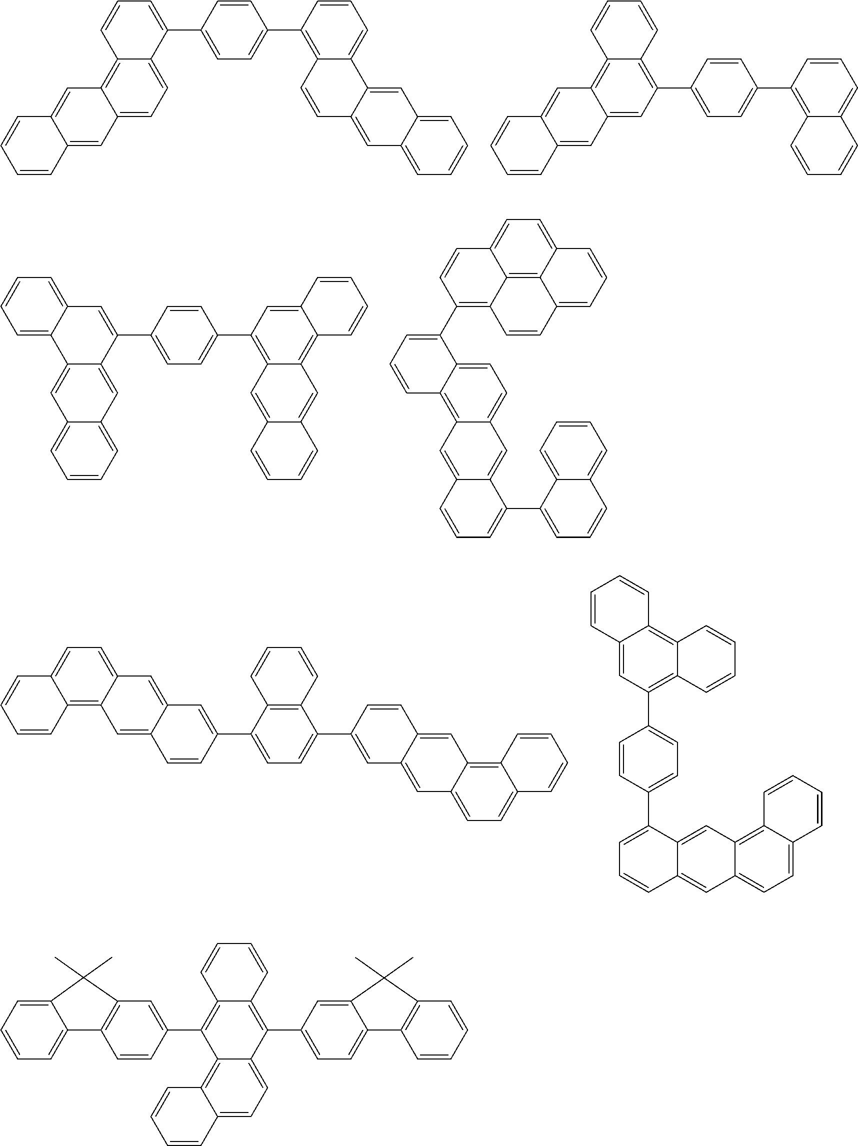

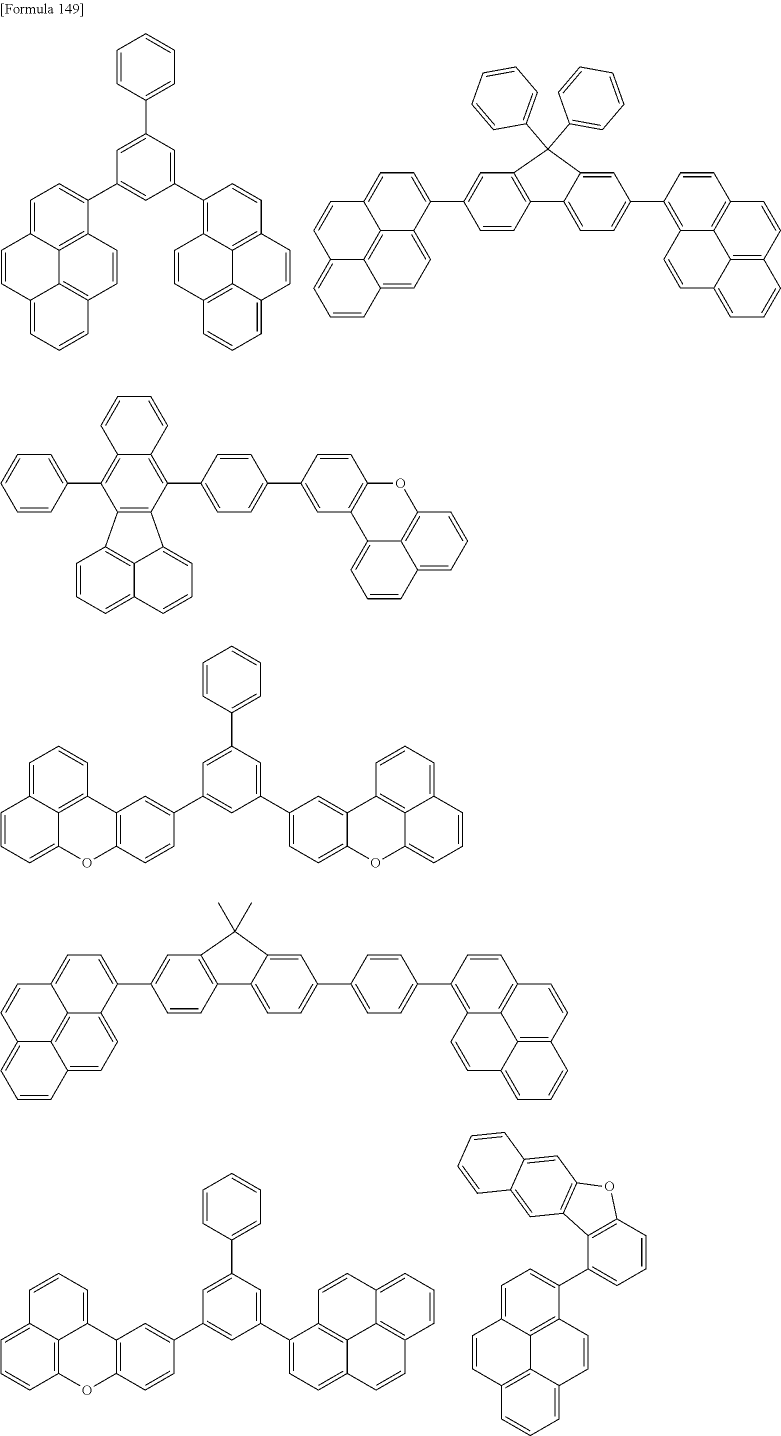

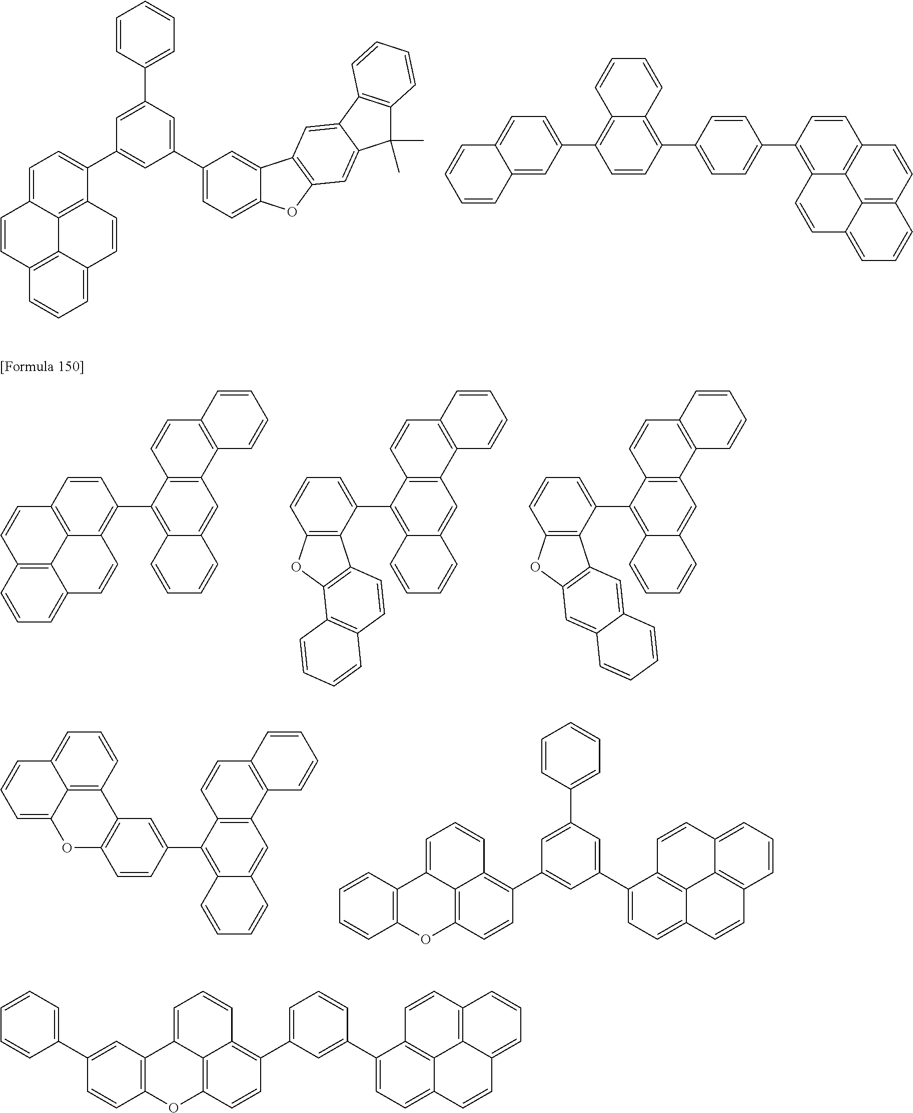

The arrangement of the composition of the exemplary embodiment will be further explained in Common Arrangements to Respective Exemplary Embodiments described later.

### Fourth Exemplary Embodiment

### Composition

A composition of a fourth exemplary embodiment contains a first compound and a second compound, in which the first compound and the second compound are mutually different compounds, an energy level of a highest occupied molecular orbital of the first compound HOMO(C1) and an energy level of a highest occupied molecular orbital of the second compound HOMO(C2) satisfy a relationship of a numerical formula (Numerical Formula A7) below, a triplet energy of the first compound T₁(C1) satisfies a relationship of a numerical formula (Numerical Formula A8) below, and a triplet energy of the second compound T₁(C2) satisfies a relationship of a numerical formula (Numerical Formula A9) below.

$$HOMO ( C 2 ) > HOMO ( C 1 ) \qquad ( Numerical Formula A7 )$$

$$1.8 eV < {T}_{1} ( C 1 ) < 2.5 eV \qquad ( Numerical Formula A8 )$$

$$1.8 eV < {T}_{1} ( C 2 ) < 2.5 eV \qquad ( Numerical Formula A9 )$$

In an exemplary arrangement of the composition of the exemplary embodiment, the triplet energy of the first compound T₁(C1) satisfies a relationship of a numerical formula (Numerical Formula A81) below, and the triplet energy of the second compound T₁(C2) satisfies a relationship of a numerical formula (Numerical Formula A91) below.

$$2. eV < {T}_{1} ( C 1 ) < 2.3 eV \qquad ( Numerical Formula A81 )$$

$$2. eV < {T}_{1} ( C 2 ) < 2.3 eV \qquad ( Numerical Formula A91 )$$

In an exemplary arrangement of the composition of the exemplary embodiment, HOMO(C2) above satisfies a relationship of a numerical formula (Numerical Formula A12) below.

$$HOMO ( C 2 ) > - 5.7 eV \qquad ( Numerical Formula A 12 )$$

In an exemplary arrangement of the composition of the exemplary embodiment, HOMO(C2) above satisfies a relationship of a numerical formula (Numerical Formula A121) below.

$$HOMO ( C 2 ) \geq - 5.6 eV \qquad ( Numerical Formula A 121 )$$

In an exemplary arrangement of the composition of the exemplary embodiment, HOMO(C2) above satisfies a relationship of a numerical formula (Numerical Formula A122) below.

$$HOMO ( C 2 ) \geq - 5.5 eV \qquad ( Numerical Formula A 122 )$$

In an exemplary arrangement of the composition of the exemplary embodiment, HOMO(C1) above satisfies a relationship of a numerical formula (Numerical Formula A13) below.

$$- 5.6 eV > HOMO ( C 1 ) \qquad ( Numerical Formula A 13 )$$

In an exemplary arrangement of the composition of the exemplary embodiment, HOMO(C1) above satisfies a relationship of a numerical formula (Numerical Formula A131) below.

$$- 5.7 eV > HOMO ( C 1 ) \qquad ( Numerical Formula A 131 )$$

In an exemplary arrangement of the composition of the exemplary embodiment, HOMO(C1) above satisfies the relationship of the numerical formula (Numerical Formula A131) and HOMO(C2) above satisfies the relationship of the numerical formula (Numerical Formula A12).

In an exemplary arrangement of the composition of the exemplary embodiment, HOMO(C1) above satisfies the relationship of the numerical formula (Numerical Formula A131) and HOMO(C2) above satisfies the relationship of the numerical formula (Numerical Formula A121).

In an exemplary arrangement of the composition of the exemplary embodiment, HOMO(C1) above satisfies the relationship of the numerical formula (Numerical Formula A131) and HOMO(C2) above satisfies the relationship of the numerical formula (Numerical Formula A122).

In an exemplary arrangement of the composition of the exemplary embodiment, the first compound and the second compound are the first compound and the second compound in the third exemplary embodiment, respectively.

The composition of the exemplary embodiment is usable for the organic EL device. The organic EL device in which the composition of the exemplary embodiment is used has improved device performance.

The emitting layer of the organic EL device can be formed by using the composition of the exemplary embodiment. Using the composition of the exemplary embodiment in the emitting layer of the organic EL device allows the organic EL device to maintain luminous efficiency and have a long lifetime.

The arrangement of the composition of the exemplary embodiment will be further explained in Common Arrangements to Respective Exemplary Embodiments described later.

### Fifth Exemplary Embodiment

### Organic Electroluminescence Device

An organic electroluminescence device of a fifth exemplary embodiment includes an anode, a cathode, and an emitting region disposed between the anode and the cathode, in which the emitting region includes a first emitting layer that contains a composition according to the third exemplary embodiment or a composition according to the fourth exemplary embodiment.

According to the exemplary embodiment, a favorable organic electroluminescence device can be provided.

### Emitting Region

The organic EL device of the exemplary embodiment includes the emitting region that includes the first emitting layer. The emitting region of the exemplary embodiment may consist of the first emitting layer, or may further include an organic layer different from the first emitting layer.

In the organic EL device of the exemplary embodiment, the first emitting layer contains a first compound and a second compound included in the composition according to the third exemplary embodiment or the composition according to the fourth exemplary embodiment.

In an exemplary arrangement of the organic EL device of the exemplary embodiment, the first compound is a first host material and the second compound is a second host material. In the organic EL device of the exemplary embodiment, the first host material and the second host material are mutually different compounds.

In an exemplary arrangement of the organic EL device of the exemplary embodiment, the first emitting layer contains a first luminescent compound.

In an exemplary arrangement of the organic EL device of the exemplary embodiment, the first luminescent compound is a compound that emits light having a maximum peak wavelength of 500 nm or less.

In an exemplary arrangement of the organic EL device of the exemplary embodiment, the emitting region includes a second emitting layer.

In an exemplary arrangement of the organic EL device of the exemplary embodiment, the second emitting layer contains a third compound, and the first compound, the second compound, and the third compound are mutually different.

In an exemplary arrangement of the organic EL device of the exemplary embodiment, a triplet energy of the first compound T₁(C1) and a triplet energy of the third host material T₁(C3) satisfy a relationship of a numerical formula (Numerical Formula A14) below, and a triplet energy of the second compound T₁(C2) and the triplet energy of the third compound T₁(C3) satisfy a relationship of a numerical formula (Numerical Formula A15) below.

$${T}_{1} ( C 1 ) > {T}_{1} ( C 3 ) \qquad ( Numerical Formula A 14 )$$

$${T}_{1} ( C 2 ) > {T}_{1} ( C 3 ) \qquad ( Numerical Formula A 15 )$$

The organic EL device according to the exemplary embodiment, in which the first emitting layer and the second emitting layer satisfying the relationships of Numerical Formula A14 and Numerical Formula A15 are layered, is expected to have the effect obtained by layering the emitting layers as in the first exemplary embodiment.

In an exemplary arrangement of the organic EL device of the exemplary embodiment, the first emitting layer is disposed between the anode and the second emitting layer.

In an exemplary arrangement of the organic EL device of the exemplary embodiment, the second emitting layer contains a second luminescent compound. In an exemplary arrangement of the organic EL device of the exemplary embodiment, the first luminescent compound and the second luminescent compound are mutually the same or different.

In an exemplary arrangement of the organic EL device of the exemplary embodiment, the second luminescent compound is a compound that emits light having a maximum peak wavelength of 500 nm or less.

In an exemplary arrangement of the organic EL device of the exemplary embodiment, a first hole transporting zone as in the second exemplary embodiment is provided between the emitting region and the anode.

In an exemplary arrangement of the organic EL device of the exemplary embodiment, a first anode side organic layer as in the second exemplary embodiment is disposed between the emitting region and the anode.

The arrangement of the organic EL device of the exemplary embodiment will be further explained in Common Arrangements to Respective Exemplary Embodiments described later.

### Common Arrangements to Respective Exemplary Embodiments

Arrangements adoptable commonly to the respective exemplary embodiments herein will be described below.

### First Emitting Layer

In the organic EL device of each of the exemplary embodiments, a single first emitting layer contains the first host material and the second host material.

In an exemplary arrangement of the organic EL device of each of the exemplary embodiments, the first emitting layer contains the first host material and the first luminescent compound. The first host material is a compound different from the second host material and the third host material.

In an exemplary arrangement of the organic EL device of each of the exemplary embodiments, the first luminescent compound is a compound that emits light having a maximum peak wavelength in a range from 430 nm to 480 nm.

In an exemplary arrangement of the organic EL device of each of the exemplary embodiments, the first luminescent compound is a fluorescent compound that emits fluorescence having a maximum peak wavelength of 500 nm or less.

In an exemplary arrangement of the organic EL device of each of the exemplary embodiments, the first luminescent compound is a fluorescent compound that emits fluorescence having a maximum peak wavelength in a range from 430 nm to 480 nm.

In an exemplary arrangement of the organic EL device of each of the exemplary embodiments, the first luminescent compound is a compound containing no azine ring structure in a molecule.

In an exemplary arrangement of the organic EL device of each of the exemplary embodiments, the first luminescent compound is not a boron-containing complex.

In an exemplary arrangement of the organic EL device of each of the exemplary embodiments, the first luminescent compound is not a complex.

In an exemplary arrangement of the organic EL device of each of the exemplary embodiments, the first emitting layer contains no metal complex.

In an exemplary arrangement of the organic EL device of each of the exemplary embodiments, the first emitting layer contains no boron-containing complex.

In an exemplary arrangement of the organic EL device of each of the exemplary embodiments, the first emitting layer contains no phosphorescent material (dopant material).

In an exemplary arrangement of the organic EL device of each of the exemplary embodiments, the first emitting layer contains no heavy-metal complex and no phosphorescent rare earth metal complex. Examples of the heavy-metal complex herein include an iridium complex, osmium complex, and platinum complex.

In an emission spectrum of the first luminescent compound, where a peak exhibiting a maximum luminous intensity is defined as a maximum peak and a height of the maximum peak is defined as 1, heights of other peaks appearing in the emission spectrum are preferably less than 0.6. It should be noted that the peaks in the emission spectrum are defined as local maximum values.

Moreover, in the emission spectrum of the first luminescent compound, the number of peaks is preferably less than three.

In an exemplary arrangement of the organic EL device of each of the exemplary embodiments, the singlet energy of the first host material S₁(H1), the singlet energy of the second host material S₁(H2), and the singlet energy of the first luminescent compound S₁(D1) satisfy relationships of a numerical formula (Numerical Formula 11) and a numerical formula (Numerical Formula 12) below.

$${S}_{1} ( H 1 ) > {S}_{1} ( D 1 ) \qquad ( Numerical Formula 11 )$$

$${S}_{1} ( H 2 ) > {S}_{1} ( D 1 ) \qquad ( Numerical Formula 12 )$$

The singlet energy S₁ means an energy difference between the lowest singlet state and the ground state.

When the first host material, the second host material, and the first luminescent compound satisfy the relationships of the numerical formula (Numerical Formula 11) and the numerical formula (Numerical Formula 12), singlet excitons generated on the first host material and the second host material easily energy-transfer from the first and second host materials to the first luminescent compound, thereby contributing to the fluorescence of the first luminescent compound.

In an exemplary arrangement of the organic EL device of each of the exemplary embodiments, the first host material, the second host material, and the first luminescent compound satisfy relationships of a numerical formula (Numerical Formula 13) and a numerical formula (Numerical Formula 14) below.

$${T}_{1} ( D 1 ) > {T}_{1} ( H 1 ) \qquad ( Numerical Formula 13 )$$

$${T}_{1} ( D 1 ) > {T}_{1} ( H 2 ) \qquad ( Numerical Formula 14 )$$

When the first host material, the second host material, and the first luminescent compound satisfy the relationships of the numerical formula (Numerical Formula 13) and the numerical formula (Numerical Formula 14), triplet excitons generated in the first emitting layer are transferred not onto the first luminescent compound having higher triplet energy but onto the first host material or the second host material, thereby being easily transferred to the second emitting layer.

In an exemplary arrangement of the organic EL device of each of the exemplary embodiments, the first host material, the second host material, the third host material, and the first luminescent compound satisfy relationships of a numerical formula (Numerical Formula 15) and a numerical formula (Numerical Formula 16) below.

$${T}_{1} ( D 1 ) > {T}_{1} ( H 1 ) > {T}_{1} ( H 3 ) \qquad ( Numerical Formula 15 )$$

$${T}_{1} ( D 1 ) > {T}_{1} ( H 2 ) > {T}_{1} ( H 3 ) \qquad ( Numerical Formula 16 )$$

In an exemplary arrangement of the organic EL device of each of the exemplary embodiments, the first emitting layer contains the first luminescent compound at 0.5 mass % or more or 1 mass % or more with respect to the total mass of the first emitting layer.

In an exemplary arrangement of the organic EL device of each of the exemplary embodiments, the first emitting layer contains the first luminescent compound at 10 mass % or less, 7 mass % or less, or 5 mass % or less with respect to the total mass of the first emitting layer.

In an exemplary arrangement of the organic EL device of each of the exemplary embodiments, a mass percentage of a mass of the second host material M_{H2} with respect to a total M_{H1}+M_{H2} of a mass of the first host material M_{H1} and the mass of the second host material M_{H2} in the first emitting layer is in a range from 5 mass % to 60 mass %.

In an exemplary arrangement of the organic EL device of each of the exemplary embodiments, the mass percentage of the mass of the second host material M_{H2} with respect to the total M_{H1}+M_{H2} is 8 mass % or more, 15 mass % or more, 25 mass % or more, or 35 mass % or more.

In an exemplary arrangement of the organic EL device of each of the exemplary embodiments, the mass percentage of the mass of the second host material M_{H2} with respect to the total M_{H1}+M_{H2} is 55 mass % or less.

In an exemplary arrangement of the organic EL device of each of the exemplary embodiments, the first emitting layer contains the first host material and the second host material, in total, at 60 mass % or more, 70 mass % or more, 80 mass % or more, 90 mass % or more, or 95 mass % or more with respect to the total mass of the first emitting layer.

In an exemplary arrangement of the organic EL device of each of the exemplary embodiments, the first emitting layer contains the first host material and the second host material, in total, at 99 mass % or less with respect to the total mass of the first emitting layer.

In an exemplary arrangement of the organic EL device of each of the exemplary embodiments, the upper limit of the total of the content ratios of the first host material, the second host material, and the first luminescent compound in the first emitting layer is 100 mass %.

In an exemplary arrangement of the organic EL device of each of the exemplary embodiments, the first emitting layer has a film thickness of 3 nm or more or 5 nm or more. A film thickness of 3 nm or more of the first emitting layer is sufficient for causing recombination of holes and electrons in the first emitting layer.

In an exemplary arrangement of the organic EL device of each of the exemplary embodiments, the first emitting layer has a film thickness of 20 nm or less or 15 nm or less. A film thickness of 20 nm or less of the first emitting layer is thin enough for transfer of triplet excitons to the second emitting layer.

In an exemplary arrangement of the organic EL device of each of the exemplary embodiments, the first host material is the first compound of the third exemplary embodiment and the second host material is the second compound.

### Second Emitting Layer

In an exemplary arrangement of the organic EL device of each of the exemplary embodiments, the second emitting layer contains the third host material and the second luminescent compound. The third host material is a compound different from the first host material and the second host material.

In an exemplary arrangement of the organic EL device of each of the exemplary embodiments, the second luminescent compound is a compound that emits light having a maximum peak wavelength in a range from 430 nm to 480 nm.

In an exemplary arrangement of the organic EL device of each of the exemplary embodiments, the second luminescent compound is a fluorescent compound that emits fluorescence having a maximum peak wavelength of 500 nm or less.

In an exemplary arrangement of the organic EL device of each of the exemplary embodiments, the second luminescent compound is a fluorescent compound that emits fluorescence having a maximum peak wavelength in a range from 430 nm to 480 nm.

In an exemplary arrangement of the organic EL device of each of the exemplary embodiments, the triplet energy of the second luminescent compound T₁(D2) and the triplet energy of the third host material T₁(H3) satisfy a relationship of a numerical formula (Numerical Formula 22) below.

$${T}_{1} ( D 2 ) > {T}_{1} ( H 3 ) \qquad ( Numerical Formula 21 )$$

When the second luminescent compound and the third host material satisfy the relationship of the numerical formula (Numerical Formula 21), in transfer of triplet excitons generated in the first emitting layer to the second emitting layer, the triplet excitons energy-transfer not onto the second luminescent compound having higher triplet energy but onto molecules of the third host material.

In addition, triplet excitons generated by recombination of holes and electrons on the third host material do not transfer to the second luminescent compound having higher triplet energy. Triplet excitons generated by recombination on molecules of the second luminescent compound quickly energy-transfer to molecules of the third host material.

Triplet excitons in the third host material do not transfer to the second luminescent compound but efficiently collide with one another on the third host material to generate singlet excitons by the TTF phenomenon.

In an exemplary arrangement of the organic EL device of each of the exemplary embodiments, a singlet energy of the third host material S₁(H3) and the singlet energy of the second luminescent compound S₁(D2) satisfy a relationship of a numerical formula (Numerical Formula 22) below.

$${S}_{1} ( H 3 ) > {S}_{1} ( D 2 ) \qquad ( Numerical Formula 22 )$$

When the second luminescent compound and the third host material satisfy the relationship of the numerical formula (Numerical Formula 22), due to the singlet energy of the second luminescent compound being smaller than the singlet energy of the third host material, singlet excitons generated by the TTF phenomenon energy-transfer from the third host material to the second luminescent compound, thereby contributing to the fluorescence of the second luminescent compound.

In an exemplary arrangement of the organic EL device of each of the exemplary embodiments, the second luminescent compound is a compound containing no azine ring structure in a molecule.

In an exemplary arrangement of the organic EL device of each of the exemplary embodiments, the second luminescent compound is not a boron-containing complex.

In an exemplary arrangement of the organic EL device of each of the exemplary embodiments, the second luminescent compound is not a complex.

In an exemplary arrangement of the organic EL device of each of the exemplary embodiments, the second emitting layer contains no metal complex.

In an exemplary arrangement of the organic EL device of each of the exemplary embodiments, the second emitting layer contains no boron-containing complex.

In an exemplary arrangement of the organic EL device of each of the exemplary embodiments, the second emitting layer contains no phosphorescent material (dopant material).

In an exemplary arrangement of the organic EL device of each of the exemplary embodiments, the second emitting layer contains no heavy-metal complex and no phosphorescent rare earth metal complex. Examples of the heavy-metal complex herein include an iridium complex, osmium complex, and platinum complex.

In an exemplary arrangement of the organic EL device of each of the exemplary embodiments, the second emitting layer contains the second luminescent compound at 0.5 mass % or more or 1 mass % or more with respect to the total mass of the second emitting layer.

In an exemplary arrangement of the organic EL device of each of the exemplary embodiments, the second emitting layer contains the second luminescent compound at 10 mass % or less, 7 mass % or less, or 5 mass % or less with respect to the total mass of the second emitting layer.

In an exemplary arrangement of the organic EL device of each of the exemplary embodiments, the second emitting layer contains the second host material at 60 mass % or more, 70 mass % or more, 80 mass % or more, 90 mass % or more, or 95 mass % or more with respect to the total mass of the second emitting layer.

In an exemplary arrangement of the organic EL device of each of the exemplary embodiments, the second emitting layer contains the second host material at 99.5 mass % or less or 99 mass % or less with respect to the total mass of the second emitting layer.

In an exemplary arrangement of the organic EL device of each of the exemplary embodiments, the second emitting layer has a film thickness of 5 nm or more or 10 nm or more. When the film thickness of the second emitting layer is 5 nm or more, it is easy to inhibit triplet excitons having transferred from the first emitting layer to the second emitting layer from returning to the first emitting layer. Further, when the film thickness of the second emitting layer is 5 nm or more, triplet excitons can be sufficiently separated from the recombination portion in the first emitting layer.

In an exemplary arrangement of the organic EL device of each of the exemplary embodiments, the second emitting layer has a film thickness of 20 nm or less. When the film thickness of the second emitting layer is 20 nm or less, a density of the triplet excitons in the second emitting layer is improved to cause the TTF phenomenon more easily.

### Third Host Material

In an exemplary arrangement of the organic EL device of each of the exemplary embodiments, the third host material is a compound represented by a formula (2) below.

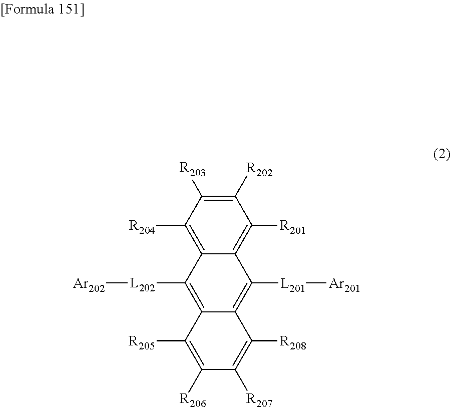

In the formula (2):
R₂₀₁ to R₂₀₈ are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a substituted or unsubstituted aralkyl group having 7 to 50 carbon atoms, a group represented by —C(═O) R₈₀₁, a group represented by —COOR₈₀₂, a halogen atom, a cyano group, a nitro group, a substituted or unsubstituted aryl group having 6 to 50 ring carbon atoms, or a substituted or unsubstituted heterocyclic group having 5 to 50 ring atoms;
L₂₀₁ and L₂₀₂ are each independently a single bond, a substituted or unsubstituted arylene group having 6 to 50 ring carbon atoms, or a substituted or unsubstituted divalent heterocyclic group having 5 to 50 ring atoms; and
Ar₂₀₁ and Ar₂₀₂ are each independently a substituted or unsubstituted aryl group having 6 to 50 ring carbon atoms, or a substituted or unsubstituted heterocyclic group having 5 to 50 ring atoms.

In the third host material, R₉₀₁, R₉₀₂, R₉₀₃, R₉₀₄, R₉₀₅, R₉₀₆, R₉₀₇, R₈₀₁ and R₈₀₂ are each independently a hydrogen atom, a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms;
when a plurality of R₉₀₁ are present, the plurality of R₉₀₁ are mutually the same or different;
when a plurality of R₉₀₂ are present, the plurality of R₉₀₂ are mutually the same or different;
when a plurality of R₉₀₃ are present, the plurality of R₉₀₃ are mutually the same or different;
when a plurality of R₉₀₄ are present, the plurality of R₉₀₄ are mutually the same or different;
when a plurality of R₉₀₅ are present, the plurality of R₉₀₅ are mutually the same or different;
when a plurality of R₉₀₆ are present, the plurality of R₉₀₆ are mutually the same or different;
when a plurality of R₉₀₇ are present, the plurality of R₉₀₇ are mutually the same or different;
when a plurality of R₈₀₁ are present, the plurality of R₈₀₁ are mutually the same or different; and
when a plurality of R₈₀₂ are present, the plurality of R₈₀₂ are mutually the same or different.

In the third host material, it is preferable that:
R₂₀₁ to R₂₀₈ are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a substituted or unsubstituted aralkyl group having 7 to 50 carbon atoms, a group represented by —C(═O) R₈₀₁, a group represented by —COOR₈₀₂, a halogen atom, a cyano group, or a nitro group;
L₂₀₁ and L₂₀₂ are each independently a single bond, a substituted or unsubstituted arylene group having 6 to 50 ring carbon atoms, or a substituted or unsubstituted divalent heterocyclic group having 5 to 50 ring atoms; and
Ar₂₀₁ and Ar₂₀₂ are each independently a substituted or unsubstituted aryl group having 6 to 50 ring carbon atoms, or a substituted or unsubstituted heterocyclic group having 5 to 50 ring atoms.

In the third host material, L₂₀₁ and L₂₀₂ are preferably each independently a single bond or a substituted or unsubstituted arylene group having 6 to 50 ring carbon atoms, and Ar₂₀₁ and Ar₂₀₂ are preferably each independently a substituted or unsubstituted aryl group having 6 to 50 ring carbon atoms.

In the third host material, Ar₂₀₁ and Ar₂₀₂ are preferably each independently a phenyl group, a naphthyl group, a phenanthryl group, a biphenyl group, a terphenyl group, a diphenylfluorenyl group, a dimethylfluorenyl group, a benzodiphenylfluorenyl group, a benzodimethylfluorenyl group, a dibenzofuranyl group, a dibenzothienyl group, a naphthobenzofuranyl group, or a naphthobenzothienyl group.

The third host material represented by the formula (2) is preferably a compound represented by a formula (201), a formula (202), a formula (203), a formula (204), a formula (205), a formula (206), a formula (207), a formula (208), or a formula (209) below.

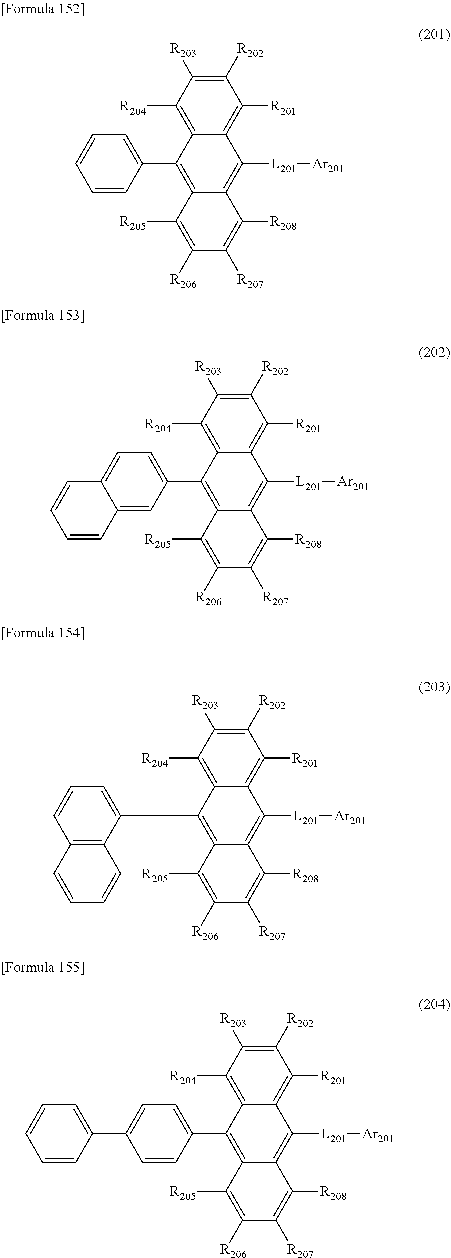

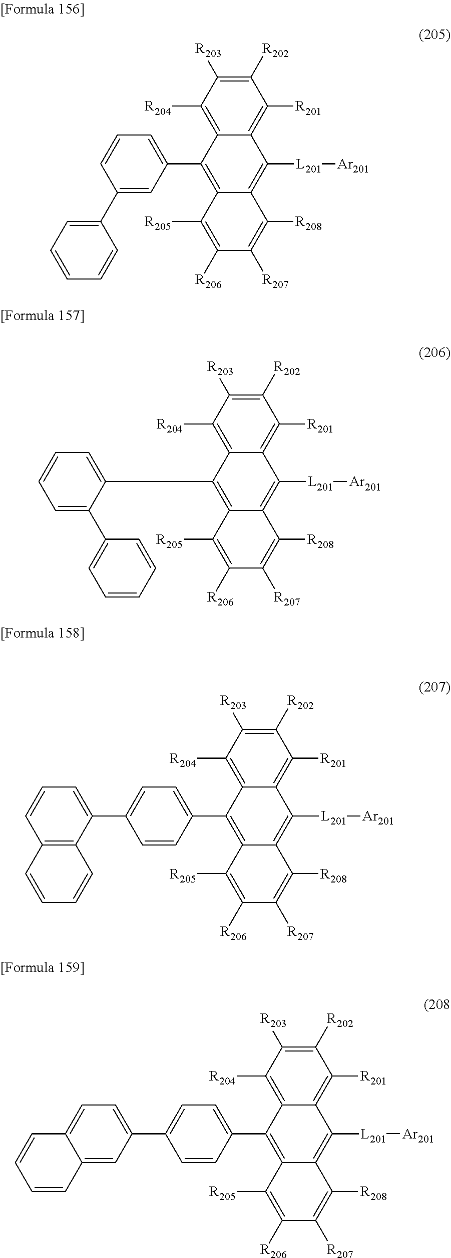

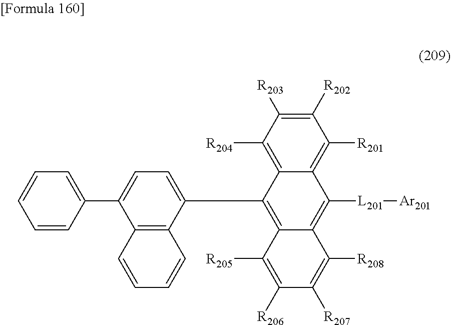

In the formulae (201) to (209), L₂₀₁ and Ar₂₀₁ respectively represent the same as L₂₀₁ and Ar₂₀₁ in the formula (2); and R₂₀₁ to R₂₀₈ each independently represent the same as R₂₀₁ to R₂₀₈ in the formula (2).

The third host material represented by the formula (2) is also preferably a compound represented by a formula (221), a formula (222), a formula (223), a formula (224), a formula (225), a formula (226), a formula (227), a formula (228), or a formula (229) below.

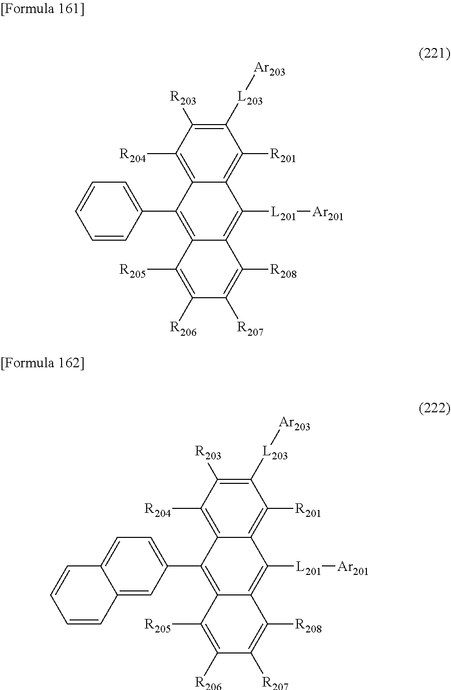

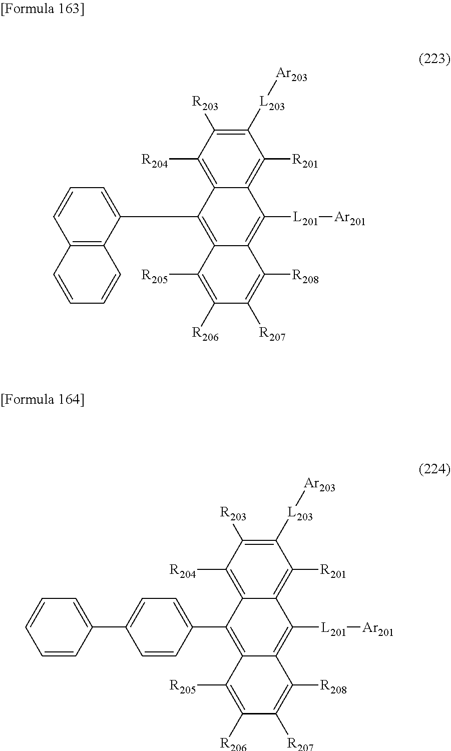

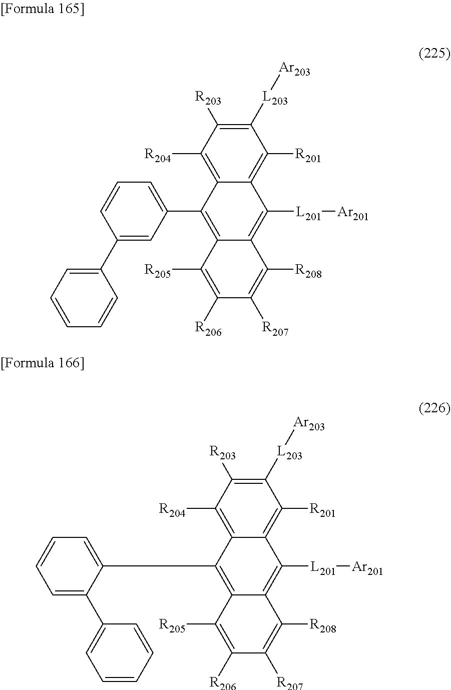

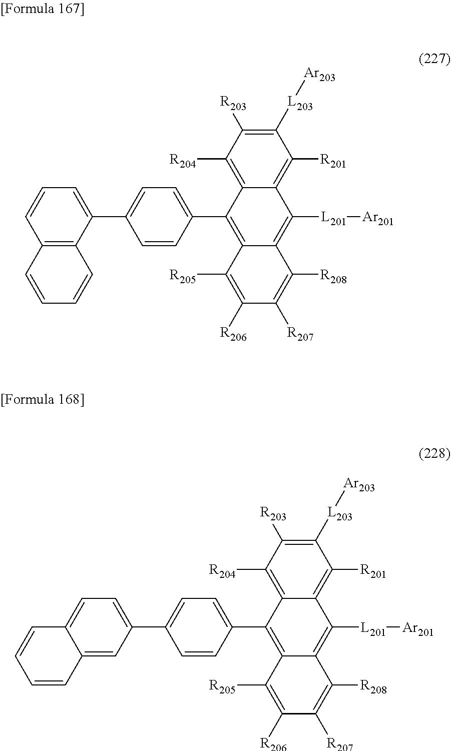

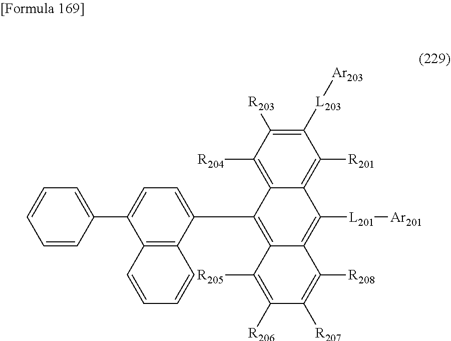

In the formulae (221), (222), (223), (224), (225), (226), (227), (228), and (229):
R₂₀₁ and R₂₀₃ to R₂₀₈ each independently represent the same as R₂₀₁ and R₂₀₃ to R₂₀₈ in the formula (2);
L₂₀₁ and Ar₂₀₁ respectively represent the same as L₂₀₁ and Ar₂₀₁ in the formula (2);
L₂₀₃ represents the same as L₂₀₁ in the formula (2);
L₂₀₃ and L₂₀₁ are mutually the same or different;
Ar₂₀₃ represents the same as Ar₂₀₁ in the formula (2); and
Ar₂₀₃ and Ar₂₀₁ are mutually the same or different.

The third host material represented by the formula (2) is also preferably a compound represented by a formula (241), a formula (242), a formula (243), a formula (244), a formula (245), a formula (246), a formula (247), a formula (248), or a formula (249) below.

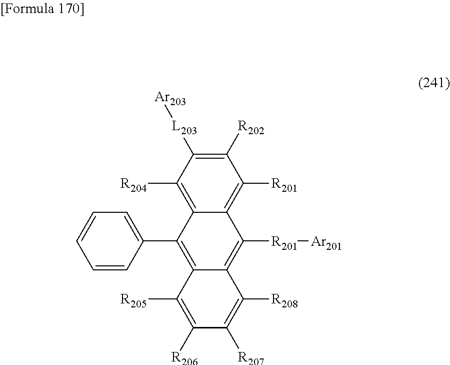

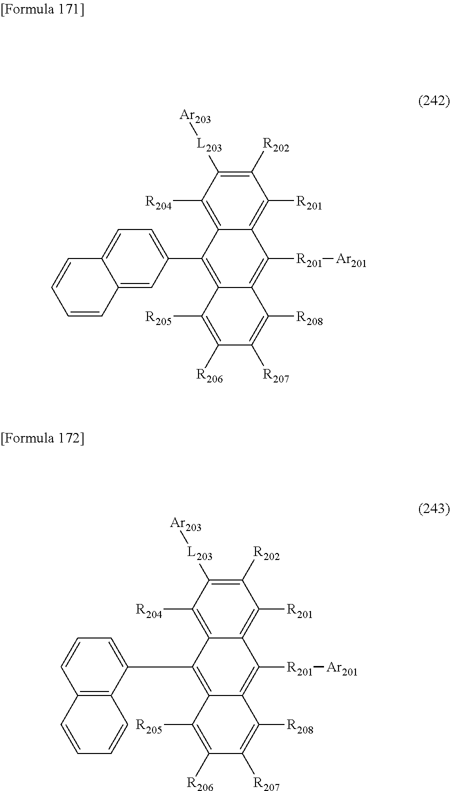

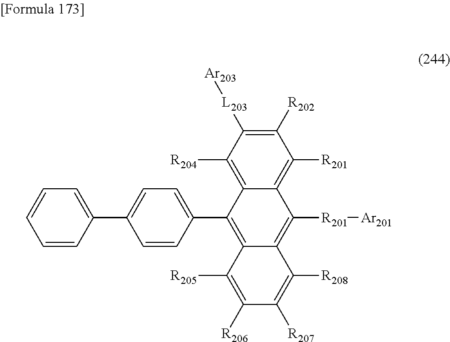

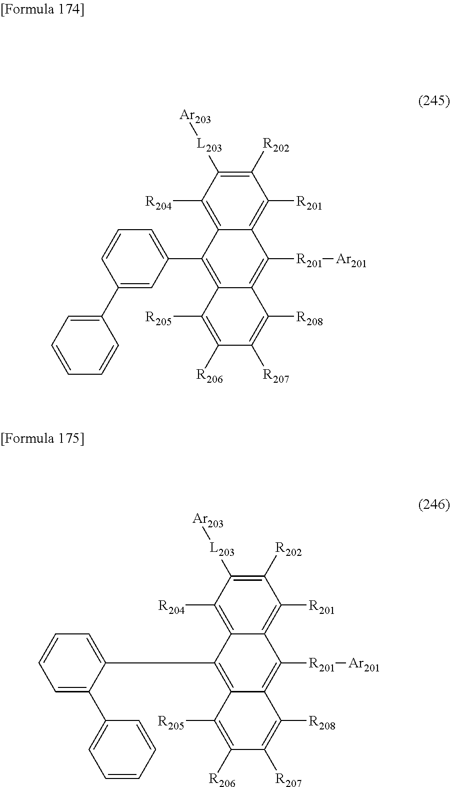

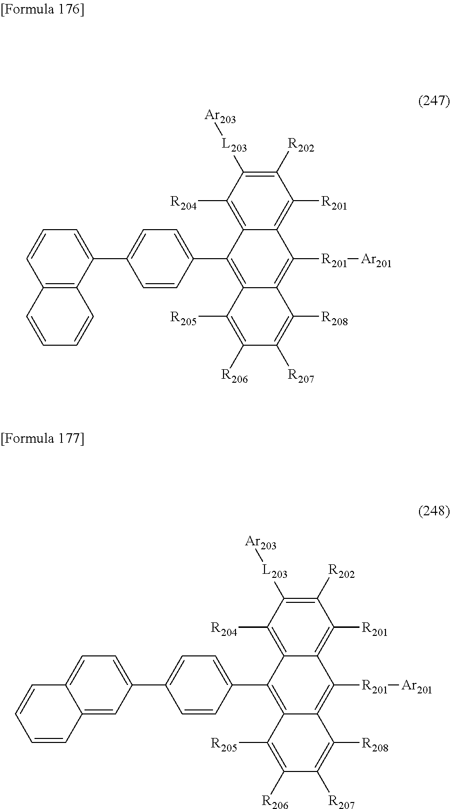

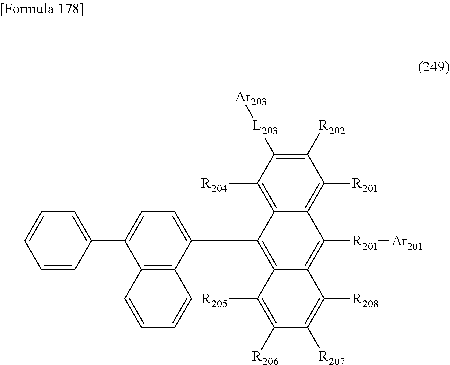

In the formulae (241), (242), (243), (244), (245), (246), (247), (248), and (249):
R₂₀₁, R₂₀₂ and R₂₀₄ to R₂₀₈ each independently represent the same as R₂₀₁, R₂₀₂ and R₂₀₄ to R₂₀₈ in the formula (2);
L₂₀₁ and Ar₂₀₁ respectively represent the same as L₂₀₁ and Ar₂₀₁ in the formula (2);
L₂₀₃ represents the same as L₂₀₁ in the formula (2);
L₂₀₃ and L₂₀₁ are mutually the same or different;
Ar₂₀₃ represents the same as Ar₂₀₁ in the formula (2); and
Ar₂₀₃ and Ar₂₀₁ are mutually the same or different.

In the third host material represented by the formula (2), R₂₀₁ to R₂₀₈ are preferably each independently a hydrogen atom, a substituted or unsubstituted alkyl group having 1 to 50 carbon atoms, a substituted or unsubstituted cycloalkyl group having 3 to 50 ring carbon atoms, or a group represented by —Si(R₉₀₁)(R₉₀₂)(R₉₀₃).

L₂₀₁ is preferably a single bond or an unsubstituted arylene group having 6 to 22 ring carbon atoms, and Ar₂₀₁ is preferably a substituted or unsubstituted aryl group having 6 to 22 ring carbon atoms.

In the third host material represented by the formula (2), R₂₀₁ to R₂₀₈ are also preferably each independently a hydrogen atom, a substituted or unsubstituted alkyl group having 1 to 50 carbon atoms, a substituted or unsubstituted cycloalkyl group having 3 to 50 ring carbon atoms, or a group represented by —Si(R₉₀₁)(R₉₀₂)(R₉₀₃).

In the third host material represented by the formula (2), R₂₀₁ to R₂₀₈ are each preferably a hydrogen atom.

In the third host material, the groups specified to be “substituted or unsubstituted” are each preferably an “unsubstituted” group.

### Method for Producing Third Host Material

The third host material can be produced by a known method. The third host material can also be produced based on a known method through a known alternative reaction using a known material(s) tailored for the target compound.

### Specific Examples of Third Host Material

Specific examples of the third host material include compounds below. However, the invention is by no means limited to the specific examples of the third host material.

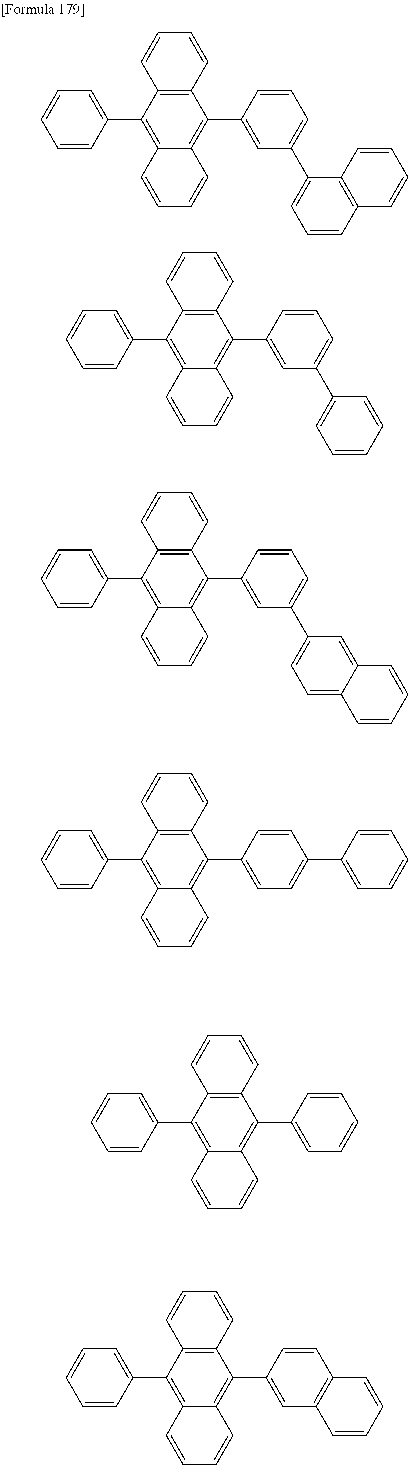

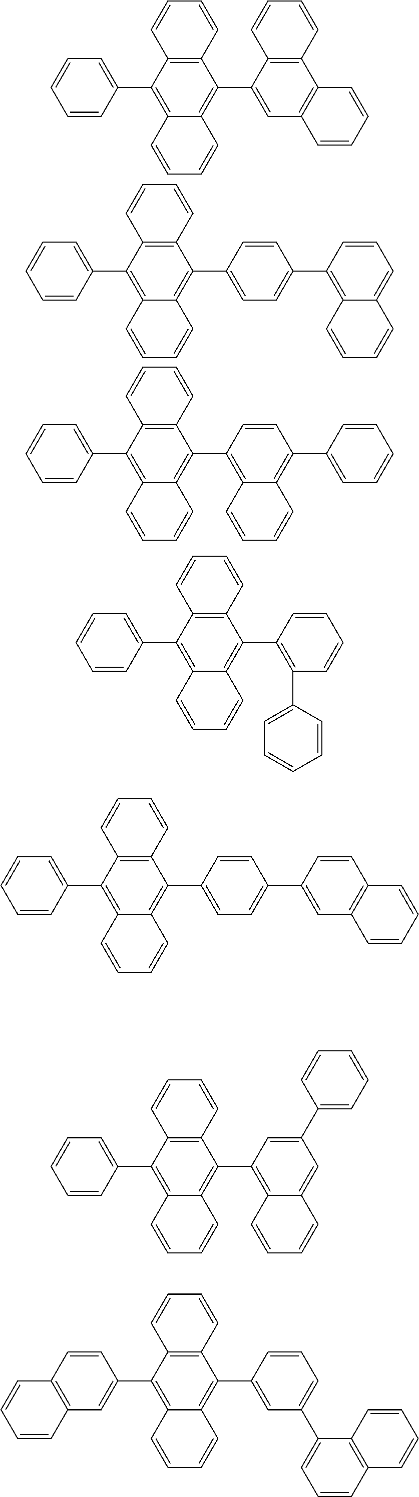

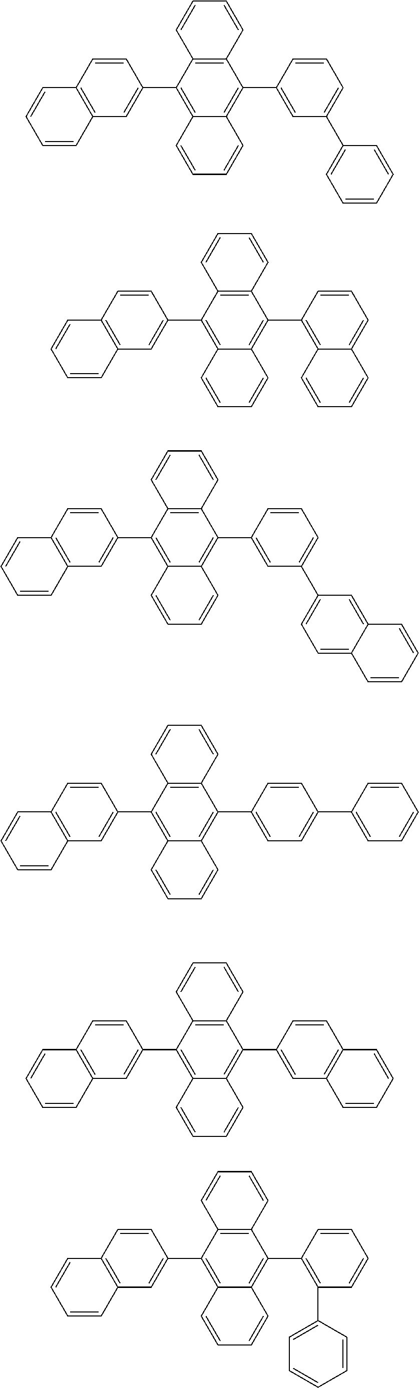

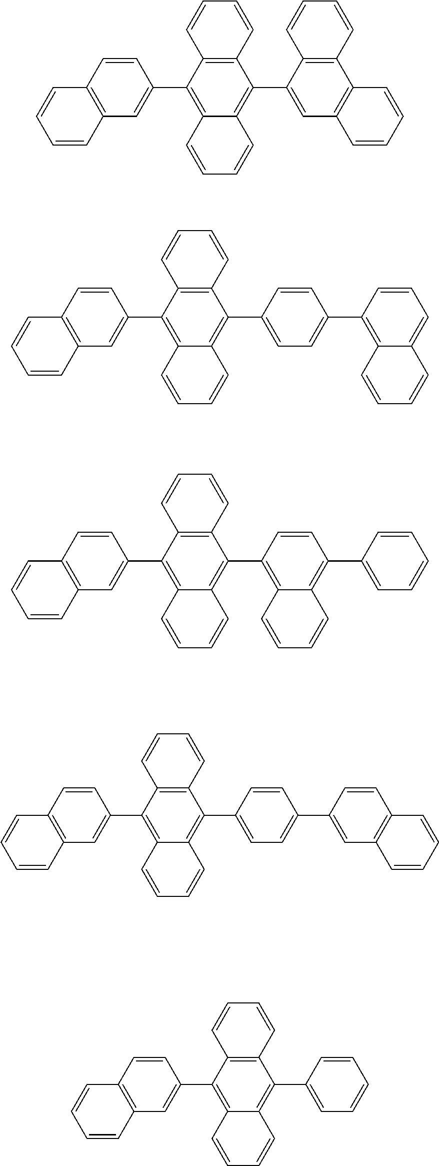

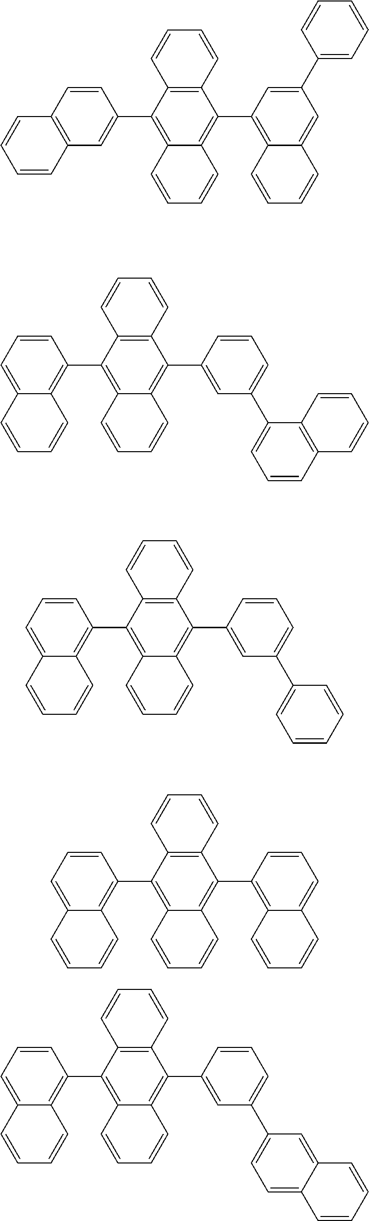

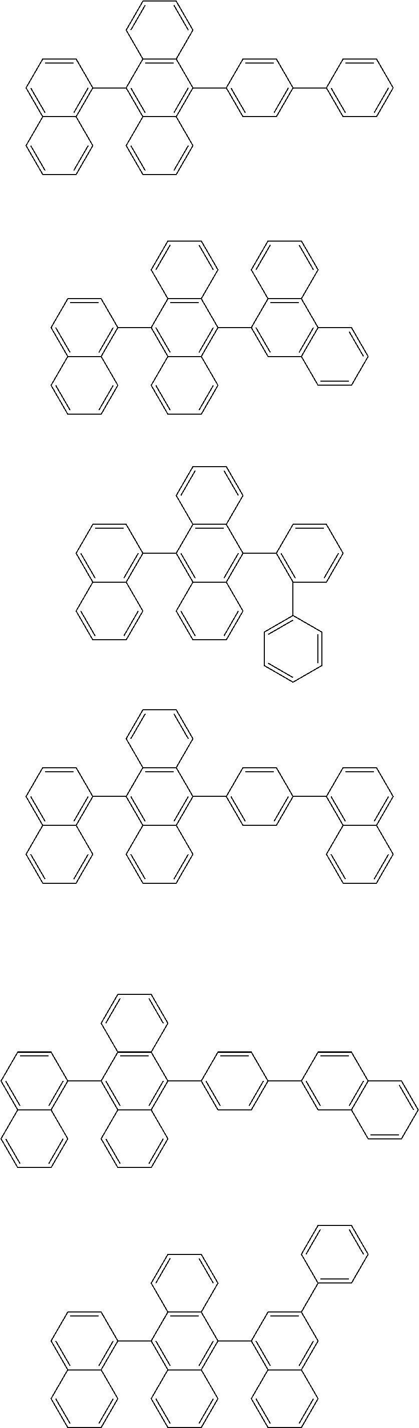

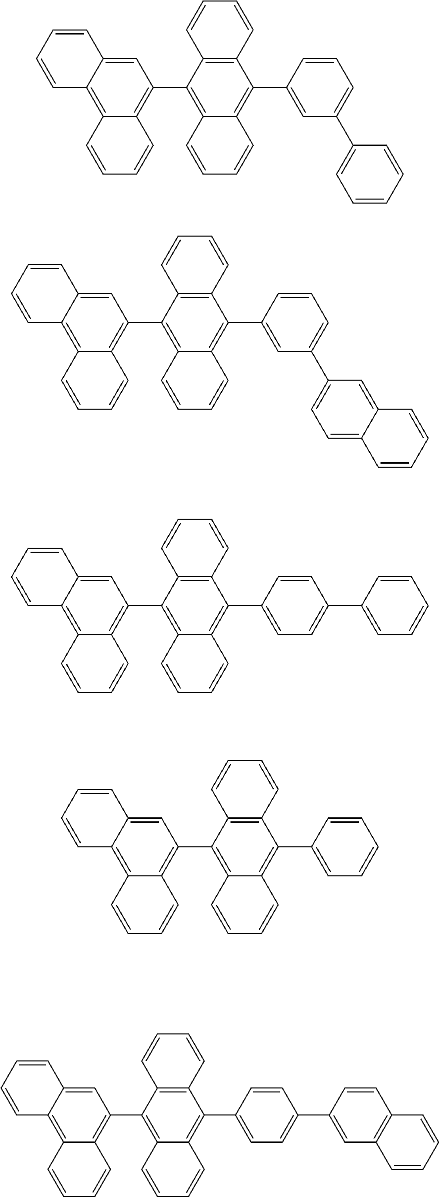

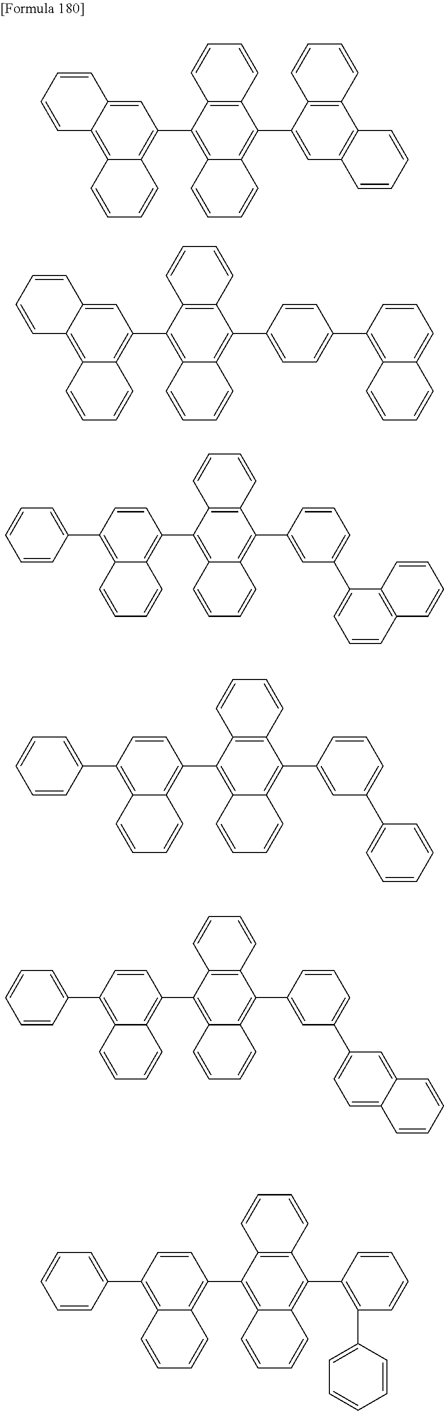

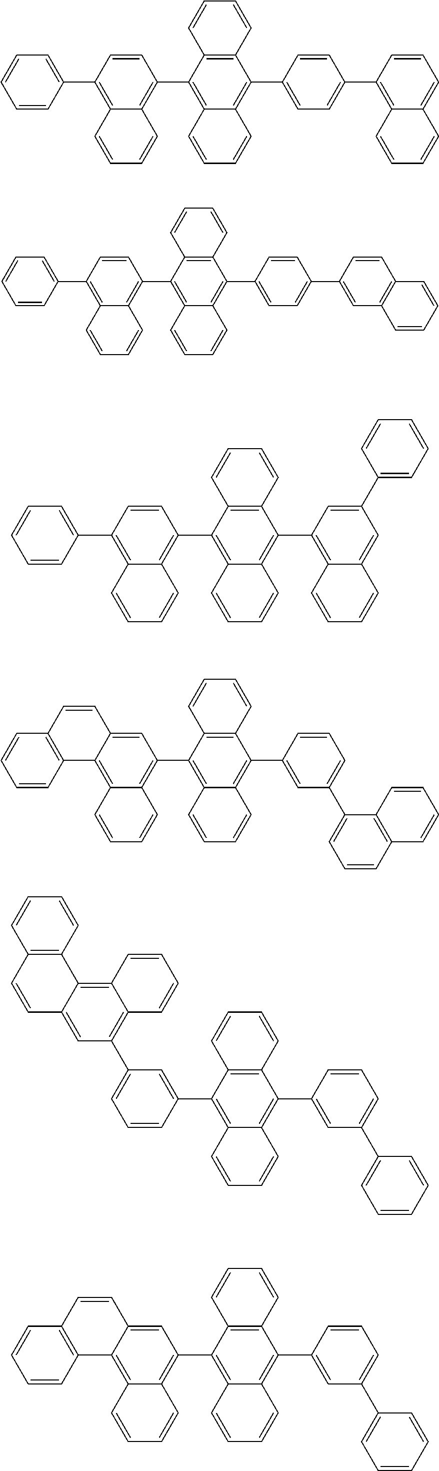

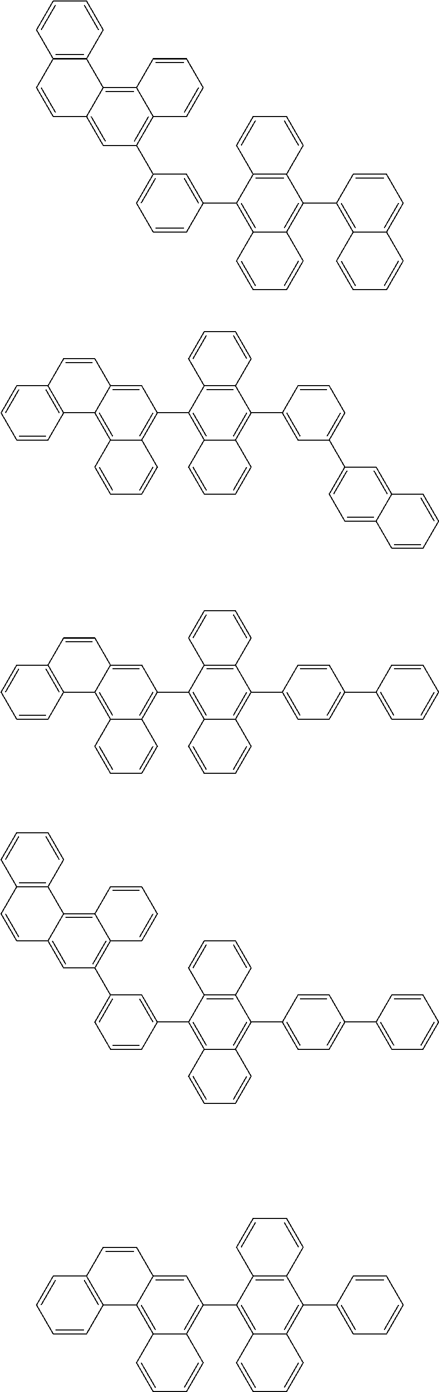

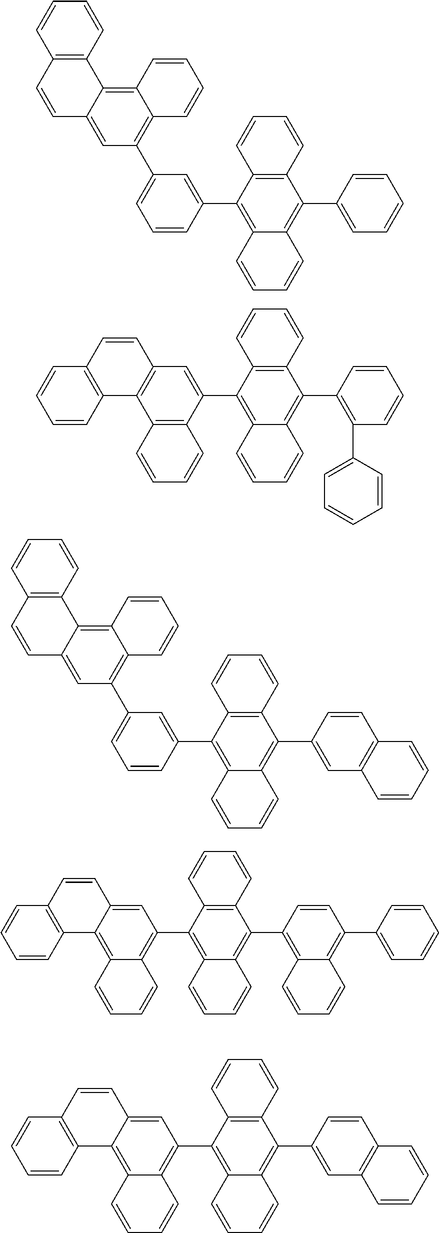

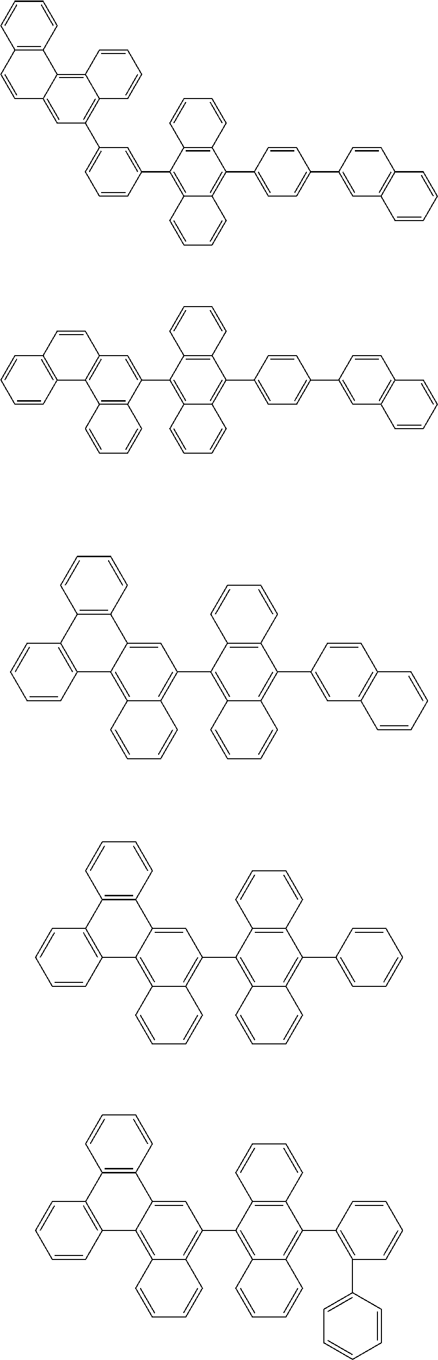

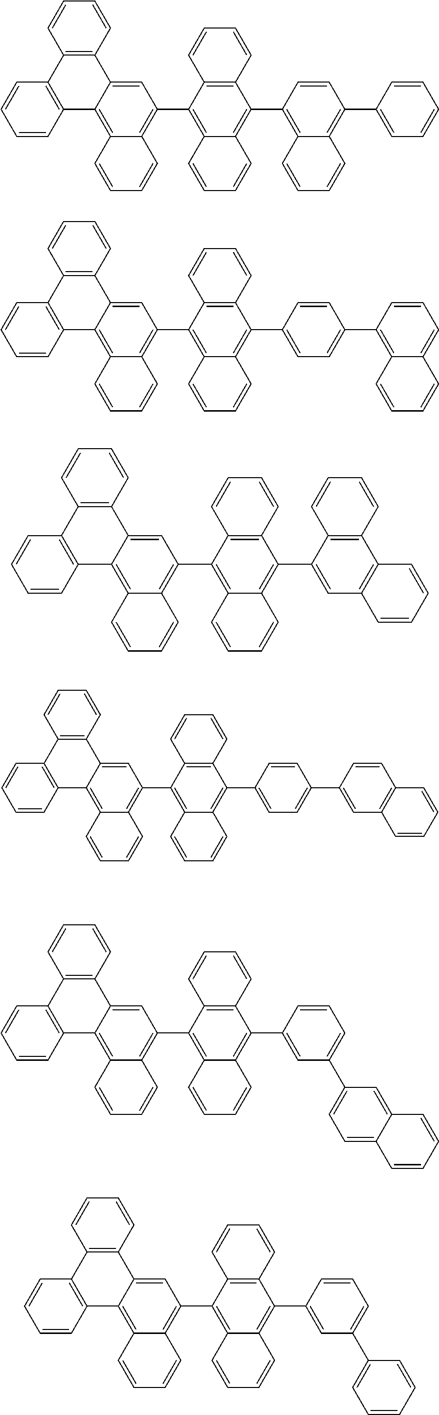

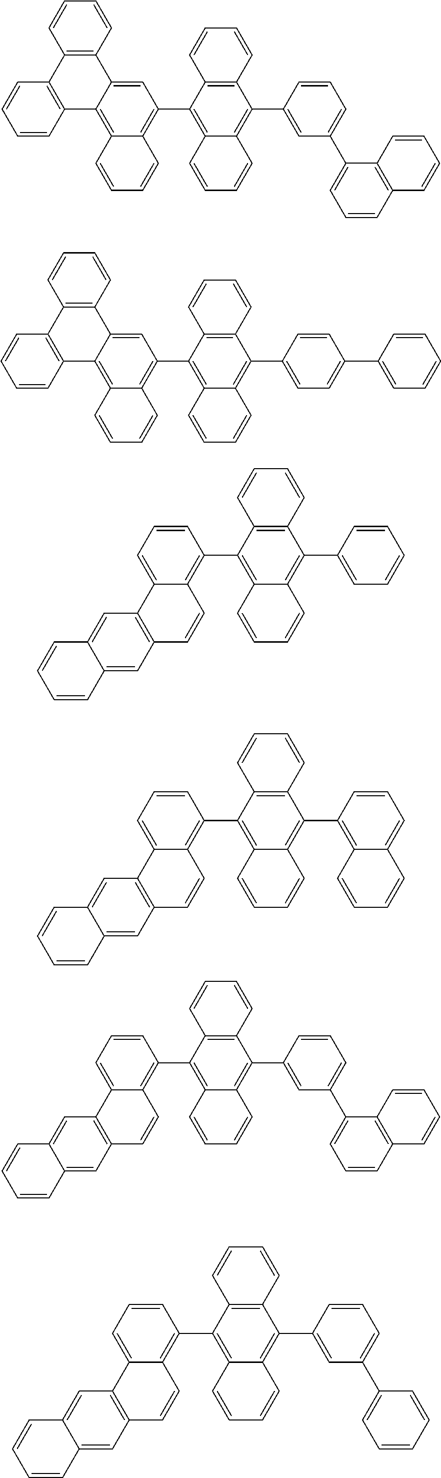

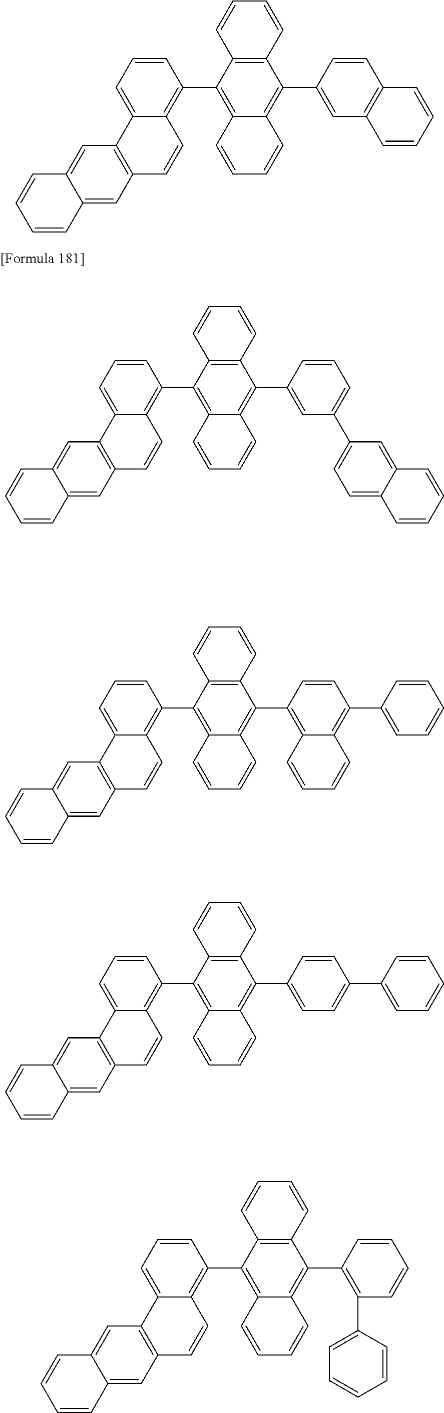

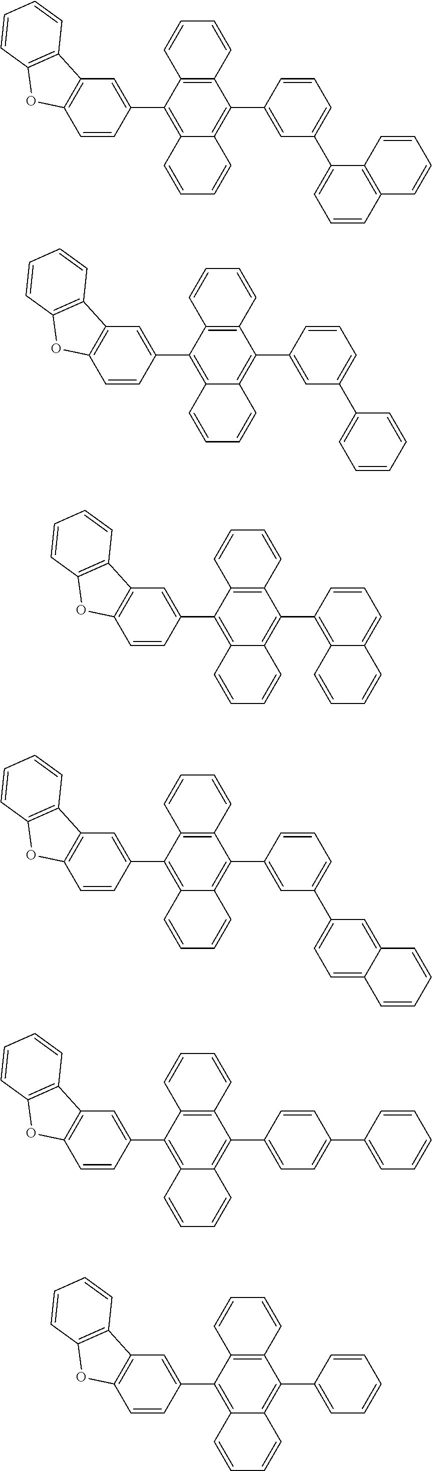 [SMILES: c1cc(-c2cccc3ccccc23)cc(-c2c3ccccc3c(-c3ccc4oc5ccccc5c4c3)c3ccccc23)c1 ; c1ccc(-c2cccc(-c3c4ccccc4c(-c4ccc5oc6ccccc6c5c4)c4ccccc34)c2)cc1 ; c1ccc2c(-c3c4ccccc4c(-c4ccc5oc6ccccc6c5c4)c4ccccc34)cccc2c1 ; c1cc(-c2ccc3ccccc3c2)cc(-c2c3ccccc3c(-c3ccc4oc5ccccc5c4c3)c3ccccc23)c1 ; c1ccc(-c2ccc(-c3c4ccccc4c(-c4ccc5oc6ccccc6c5c4)c4ccccc34)cc2)cc1 ; c1ccc(-c2c3ccccc3c(-c3ccc4oc5ccccc5c4c3)c3ccccc23)cc1]

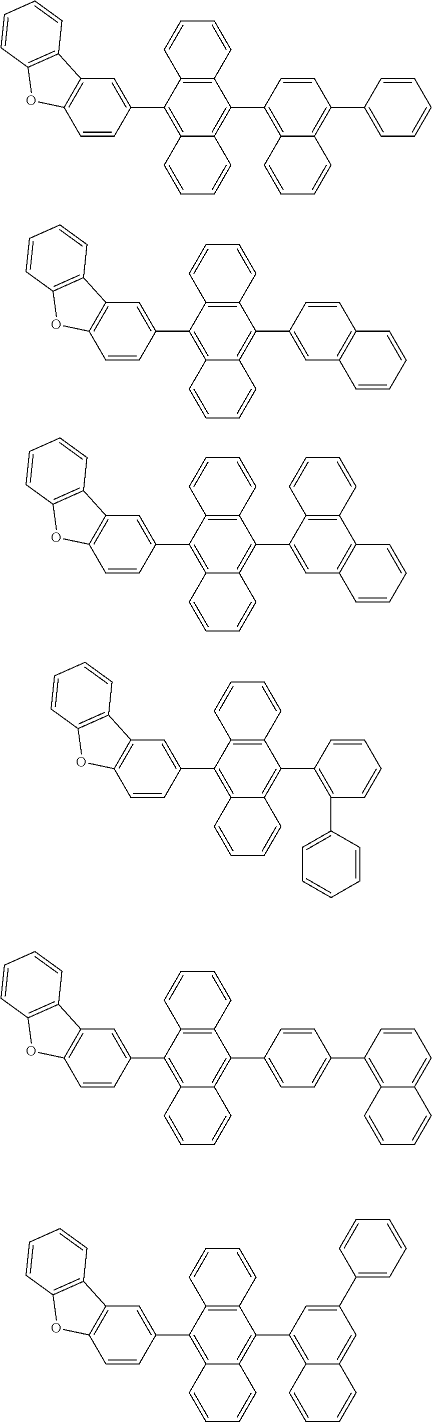

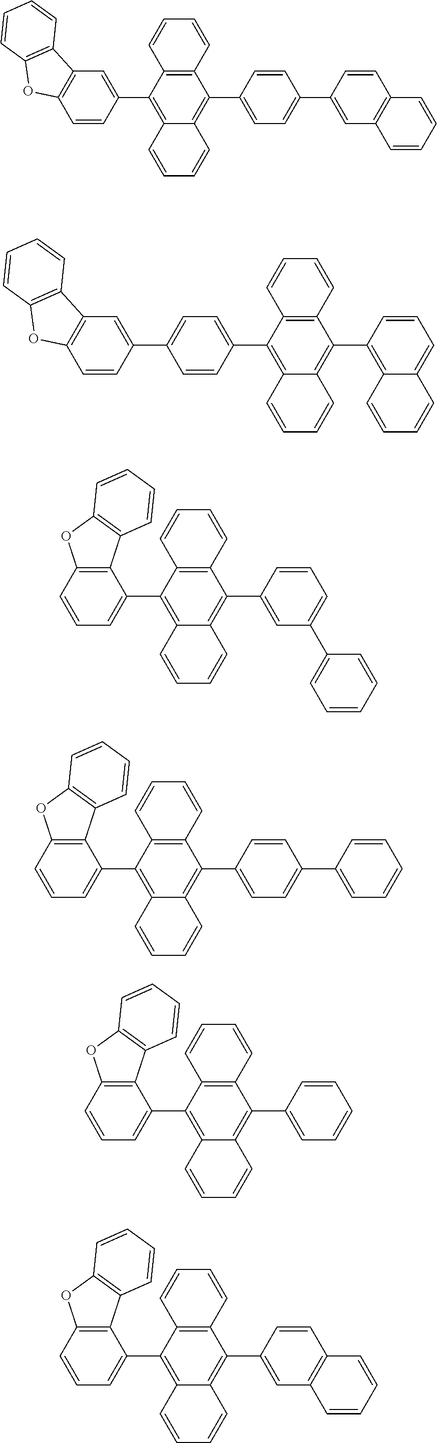

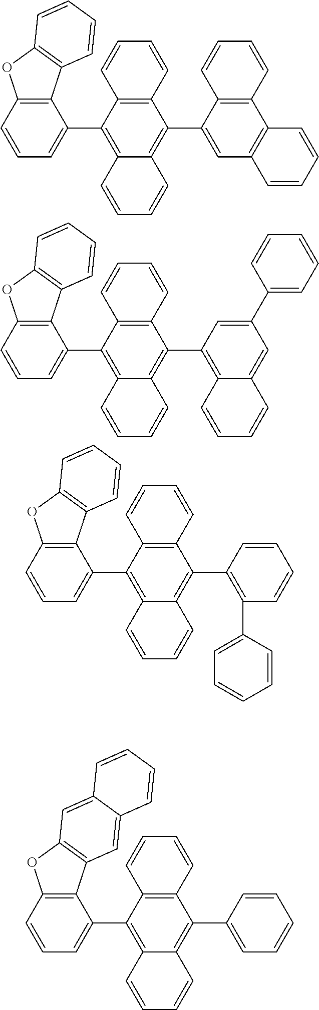

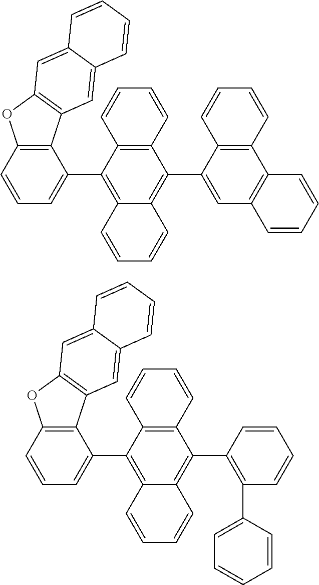

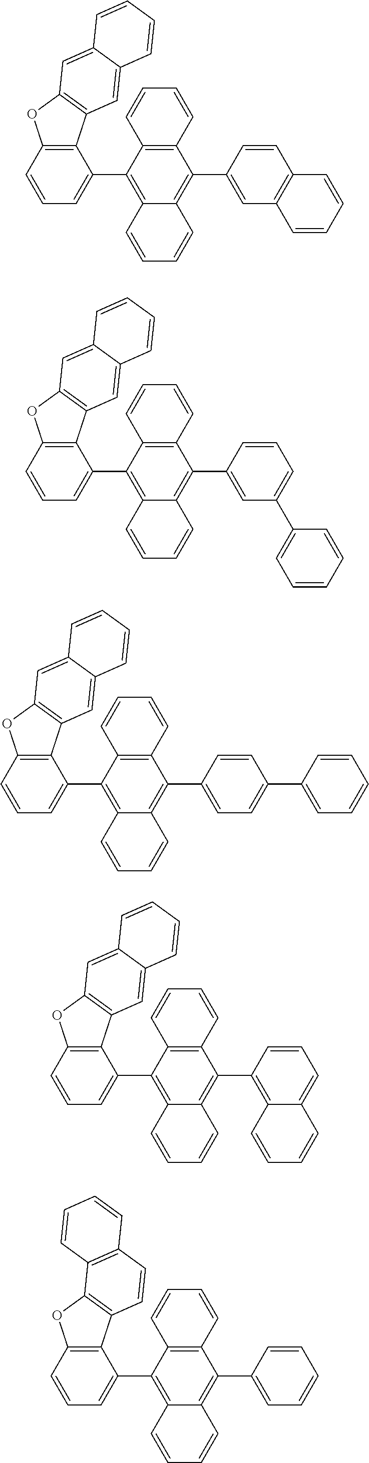

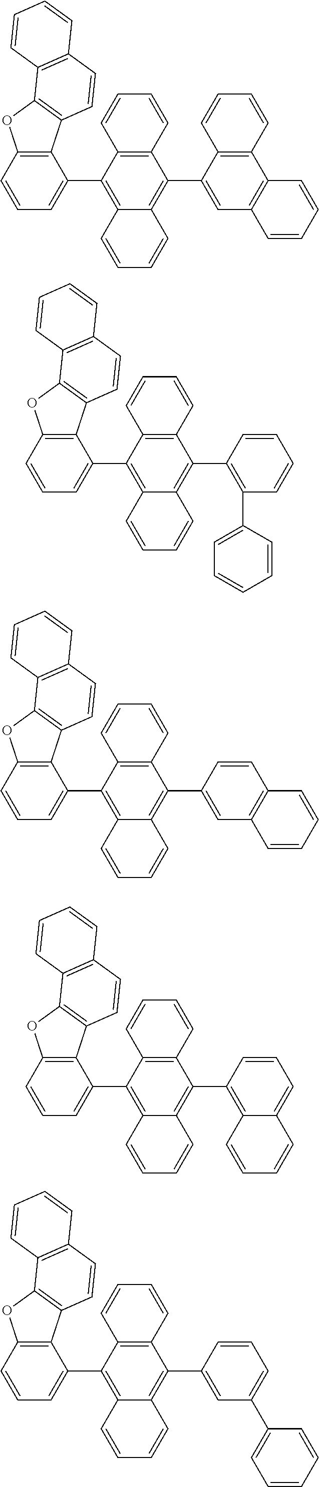

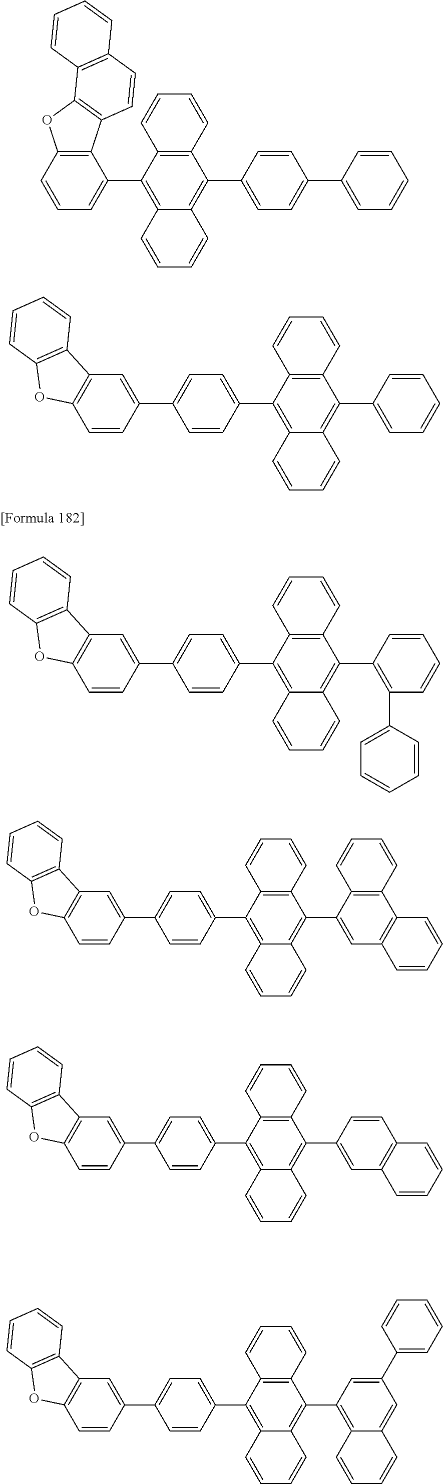 [SMILES: c1ccc(-c2ccc(-c3c4ccccc4c(-c4cccc5oc6c7ccccc7ccc6c45)c4ccccc34)cc2)cc1 ; c1ccc(-c2c3ccccc3c(-c3ccc(-c4ccc5oc6ccccc6c5c4)cc3)c3ccccc23)cc1 ; c1ccc(-c2ccccc2-c2c3ccccc3c(-c3ccc(-c4ccc5oc6ccccc6c5c4)cc3)c3ccccc23)cc1 ; c1ccc2c(c1)cc(-c1c3ccccc3c(-c3ccc(-c4ccc5oc6ccccc6c5c4)cc3)c3ccccc13)c1ccccc12 ; c1ccc2cc(-c3c4ccccc4c(-c4ccc(-c5ccc6oc7ccccc7c6c5)cc4)c4ccccc34)ccc2c1 ; c1ccc(-c2cc(-c3c4ccccc4c(-c4ccc(-c5ccc6oc7ccccc7c6c5)cc4)c4ccccc34)c3ccccc3c2)cc1]

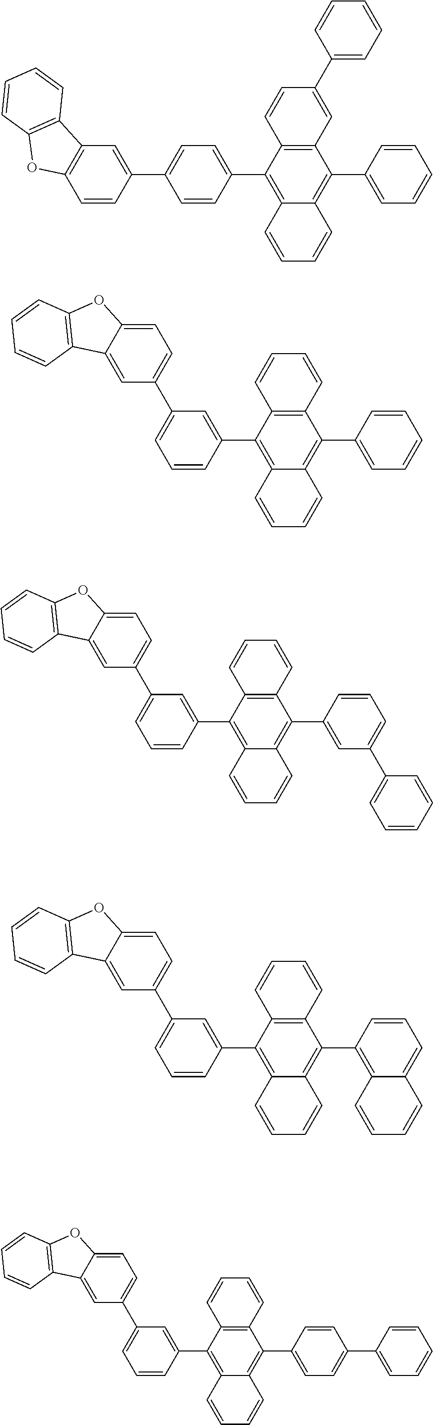 [SMILES: c1ccc(-c2ccc3c(-c4ccc(-c5ccc6oc7ccccc7c6c5)cc4)c4ccccc4c(-c4ccccc4)c3c2)cc1 ; c1ccc(-c2c3ccccc3c(-c3cccc(-c4ccc5oc6ccccc6c5c4)c3)c3ccccc23)cc1 ; c1ccc(-c2cccc(-c3c4ccccc4c(-c4cccc(-c5ccc6oc7ccccc7c6c5)c4)c4ccccc34)c2)cc1 ; c1cc(-c2ccc3oc4ccccc4c3c2)cc(-c2c3ccccc3c(-c3cccc4ccccc34)c3ccccc23)c1 ; c1ccc(-c2ccc(-c3c4ccccc4c(-c4cccc(-c5ccc6oc7ccccc7c6c5)c4)c4ccccc34)cc2)cc1]

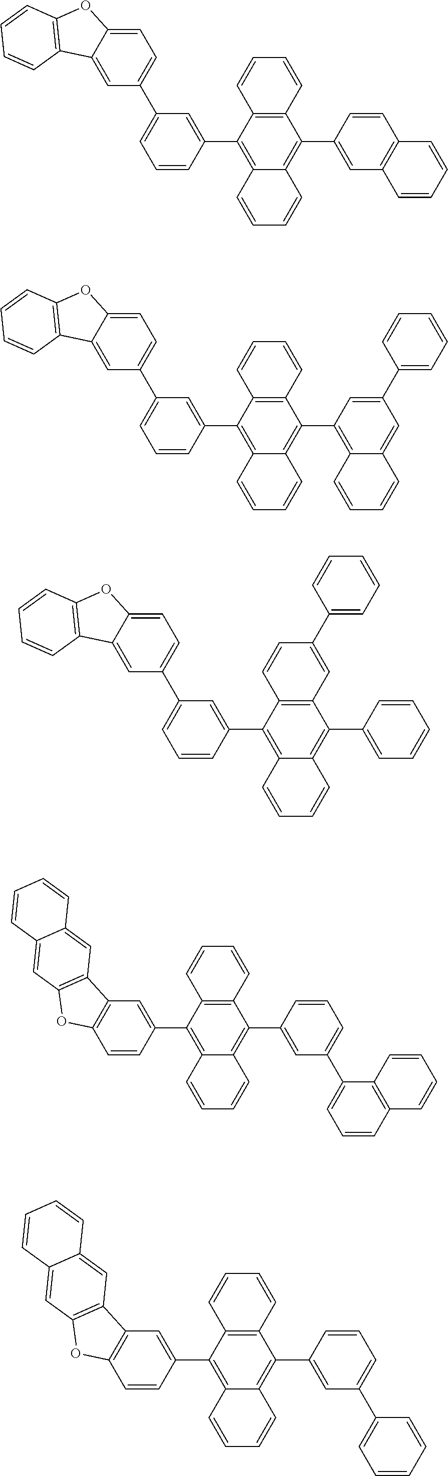 [SMILES: c1cc(-c2ccc3oc4ccccc4c3c2)cc(-c2c3ccccc3c(-c3ccc4ccccc4c3)c3ccccc23)c1 ; c1ccc(-c2cc(-c3c4ccccc4c(-c4cccc(-c5ccc6oc7ccccc7c6c5)c4)c4ccccc34)c3ccccc3c2)cc1 ; c1ccc(-c2ccc3c(-c4cccc(-c5ccc6oc7ccccc7c6c5)c4)c4ccccc4c(-c4ccccc4)c3c2)cc1 ; c1cc(-c2cccc3ccccc23)cc(-c2c3ccccc3c(-c3ccc4oc5cc6ccccc6cc5c4c3)c3ccccc23)c1 ; c1ccc(-c2cccc(-c3c4ccccc4c(-c4ccc5oc6cc7ccccc7cc6c5c4)c4ccccc34)c2)cc1]

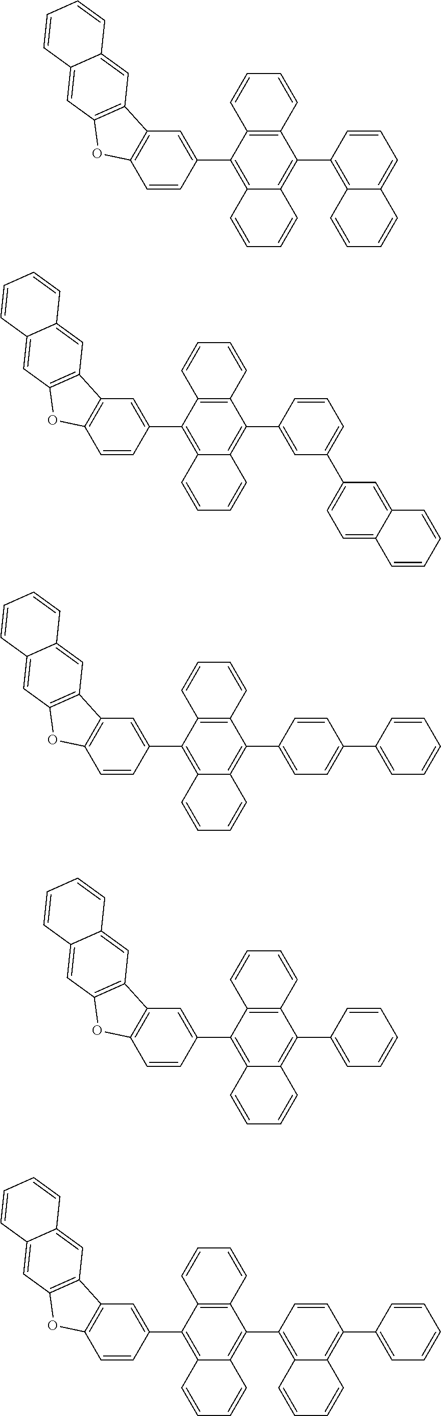 [SMILES: c1ccc2cc3c(cc2c1)oc1ccc(-c2c4ccccc4c(-c4cccc5ccccc45)c4ccccc24)cc13 ; c1cc(-c2ccc3ccccc3c2)cc(-c2c3ccccc3c(-c3ccc4oc5cc6ccccc6cc5c4c3)c3ccccc23)c1 ; c1ccc(-c2ccc(-c3c4ccccc4c(-c4ccc5oc6cc7ccccc7cc6c5c4)c4ccccc34)cc2)cc1 ; c1ccc(-c2c3ccccc3c(-c3ccc4oc5cc6ccccc6cc5c4c3)c3ccccc23)cc1 ; c1ccc(-c2ccc(-c3c4ccccc4c(-c4ccc5oc6cc7ccccc7cc6c5c4)c4ccccc34)c3ccccc23)cc1]

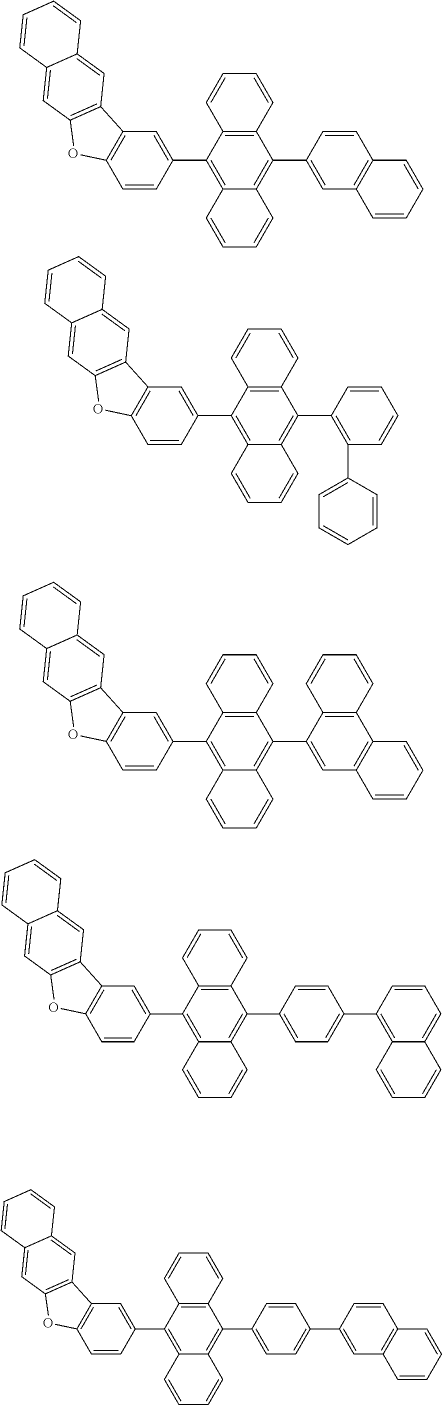 [SMILES: c1ccc2cc(-c3c4ccccc4c(-c4ccc5oc6cc7ccccc7cc6c5c4)c4ccccc34)ccc2c1 ; c1ccc(-c2ccccc2-c2c3ccccc3c(-c3ccc4oc5cc6ccccc6cc5c4c3)c3ccccc23)cc1 ; c1ccc2cc3c(cc2c1)oc1ccc(-c2c4ccccc4c(-c4cc5ccccc5c5ccccc45)c4ccccc24)cc13 ; c1ccc2cc3c(cc2c1)oc1ccc(-c2c4ccccc4c(-c4ccc(-c5cccc6ccccc56)cc4)c4ccccc24)cc13 ; c1ccc2cc(-c3ccc(-c4c5ccccc5c(-c5ccc6oc7cc8ccccc8cc7c6c5)c5ccccc45)cc3)ccc2c1]

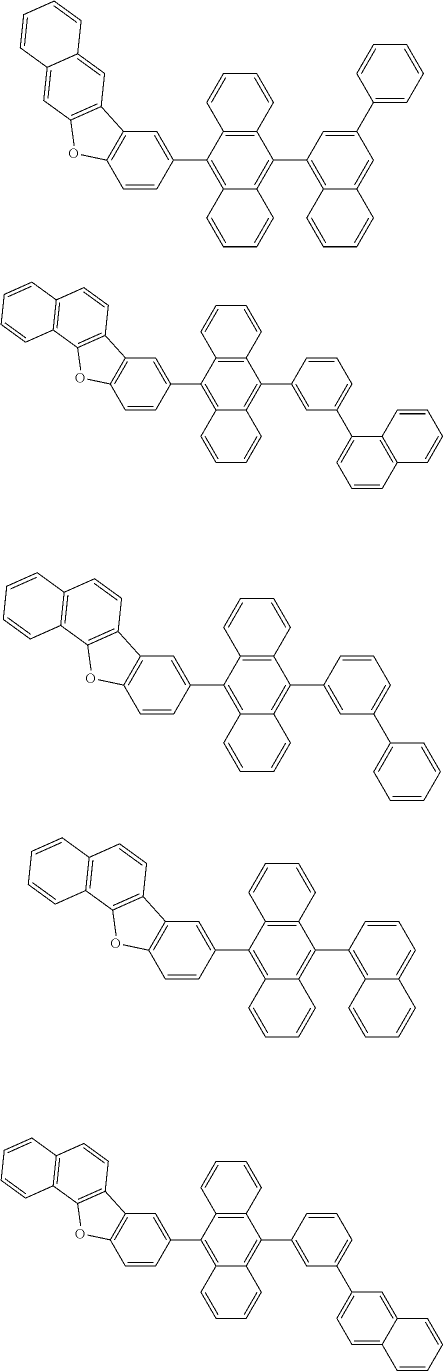 [SMILES: c1ccc(-c2cc(-c3c4ccccc4c(-c4ccc5oc6cc7ccccc7cc6c5c4)c4ccccc34)c3ccccc3c2)cc1 ; c1cc(-c2cccc3ccccc23)cc(-c2c3ccccc3c(-c3ccc4oc5c6ccccc6ccc5c4c3)c3ccccc23)c1 ; c1ccc(-c2cccc(-c3c4ccccc4c(-c4ccc5oc6c7ccccc7ccc6c5c4)c4ccccc34)c2)cc1 ; c1ccc2c(-c3c4ccccc4c(-c4ccc5oc6c7ccccc7ccc6c5c4)c4ccccc34)cccc2c1 ; c1cc(-c2ccc3ccccc3c2)cc(-c2c3ccccc3c(-c3ccc4oc5c6ccccc6ccc5c4c3)c3ccccc23)c1]

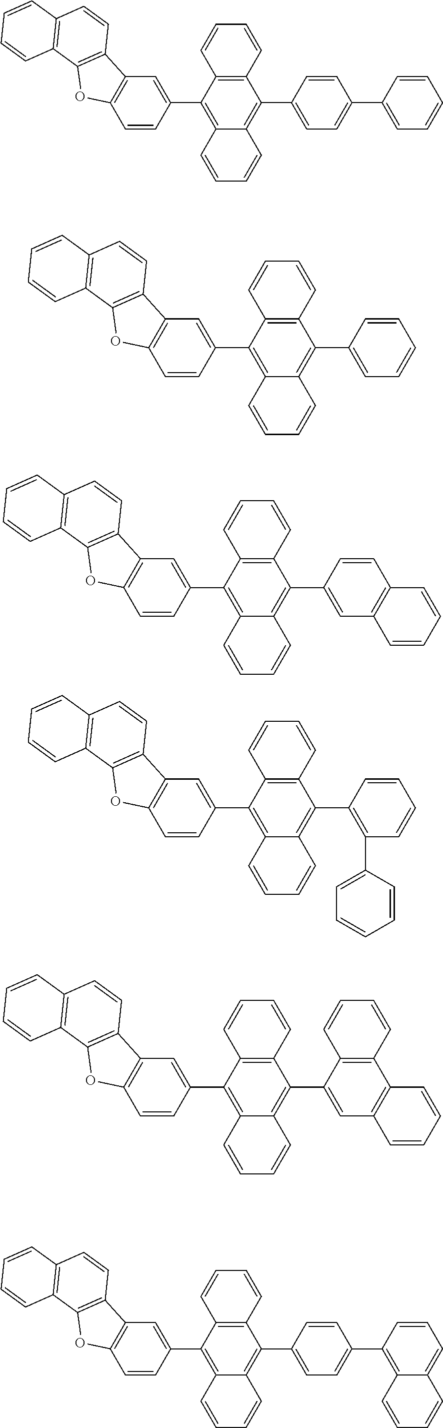 [SMILES: c1ccc(-c2ccc(-c3c4ccccc4c(-c4ccc5oc6c7ccccc7ccc6c5c4)c4ccccc34)cc2)cc1 ; c1ccc(-c2c3ccccc3c(-c3ccc4oc5c6ccccc6ccc5c4c3)c3ccccc23)cc1 ; c1ccc2cc(-c3c4ccccc4c(-c4ccc5oc6c7ccccc7ccc6c5c4)c4ccccc34)ccc2c1 ; c1ccc(-c2ccccc2-c2c3ccccc3c(-c3ccc4oc5c6ccccc6ccc5c4c3)c3ccccc23)cc1 ; c1ccc2c(c1)cc(-c1c3ccccc3c(-c3ccc4oc5c6ccccc6ccc5c4c3)c3ccccc13)c1ccccc12 ; c1ccc2c(-c3ccc(-c4c5ccccc5c(-c5ccc6oc7c8ccccc8ccc7c6c5)c5ccccc45)cc3)cccc2c1]

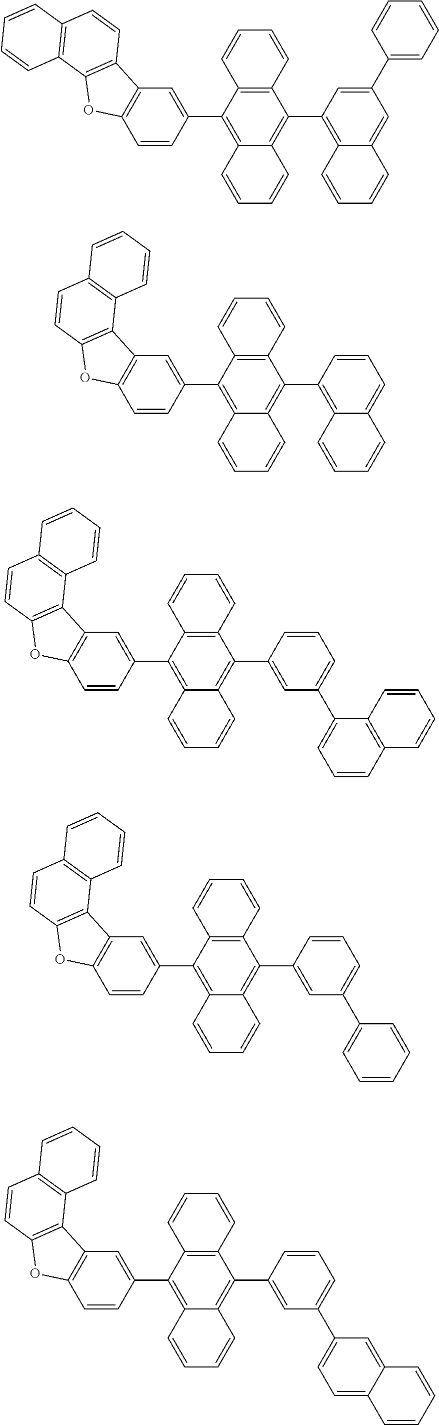 [SMILES: c1ccc(-c2cc(-c3c4ccccc4c(-c4ccc5oc6c7ccccc7ccc6c5c4)c4ccccc34)c3ccccc3c2)cc1 ; c1ccc2c(-c3c4ccccc4c(-c4ccc5oc6ccc7ccccc7c6c5c4)c4ccccc34)cccc2c1 ; c1cc(-c2cccc3ccccc23)cc(-c2c3ccccc3c(-c3ccc4oc5ccc6ccccc6c5c4c3)c3ccccc23)c1 ; c1ccc(-c2cccc(-c3c4ccccc4c(-c4ccc5oc6ccc7ccccc7c6c5c4)c4ccccc34)c2)cc1 ; c1cc(-c2ccc3ccccc3c2)cc(-c2c3ccccc3c(-c3ccc4oc5ccc6ccccc6c5c4c3)c3ccccc23)c1]

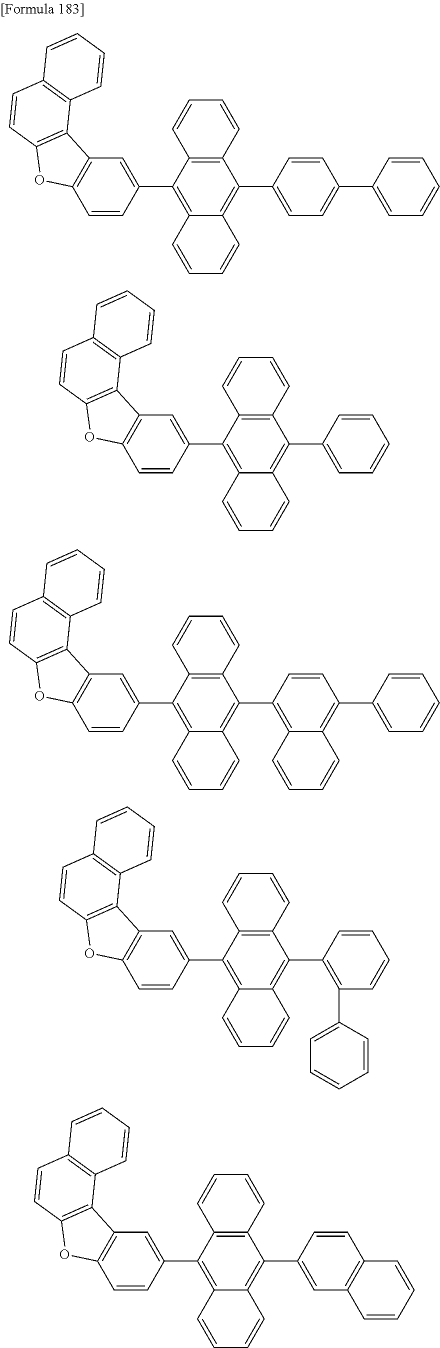 [SMILES: c1ccc(-c2ccc(-c3c4ccccc4c(-c4ccc5oc6ccc7ccccc7c6c5c4)c4ccccc34)cc2)cc1 ; c1ccc(-c2c3ccccc3c(-c3ccc4oc5ccc6ccccc6c5c4c3)c3ccccc23)cc1 ; c1ccc(-c2ccc(-c3c4ccccc4c(-c4ccc5oc6ccc7ccccc7c6c5c4)c4ccccc34)c3ccccc23)cc1 ; c1ccc(-c2ccccc2-c2c3ccccc3c(-c3ccc4oc5ccc6ccccc6c5c4c3)c3ccccc23)cc1 ; c1ccc2cc(-c3c4ccccc4c(-c4ccc5oc6ccc7ccccc7c6c5c4)c4ccccc34)ccc2c1]

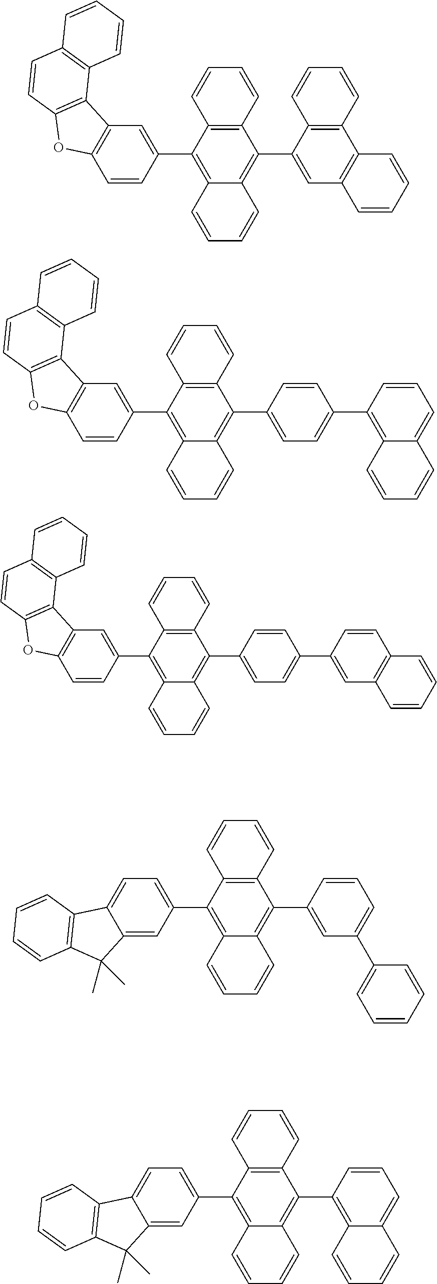

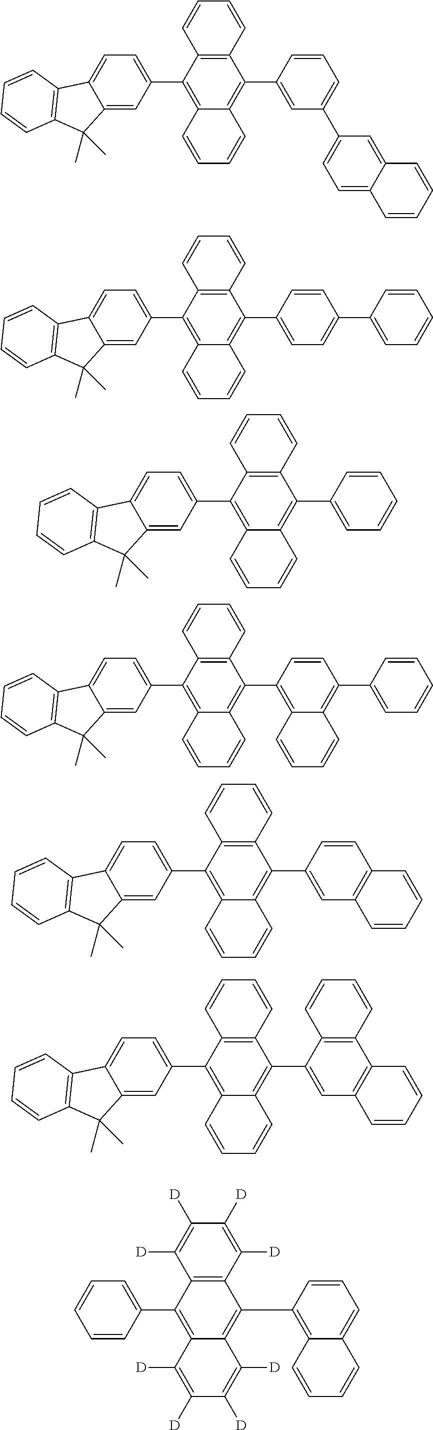

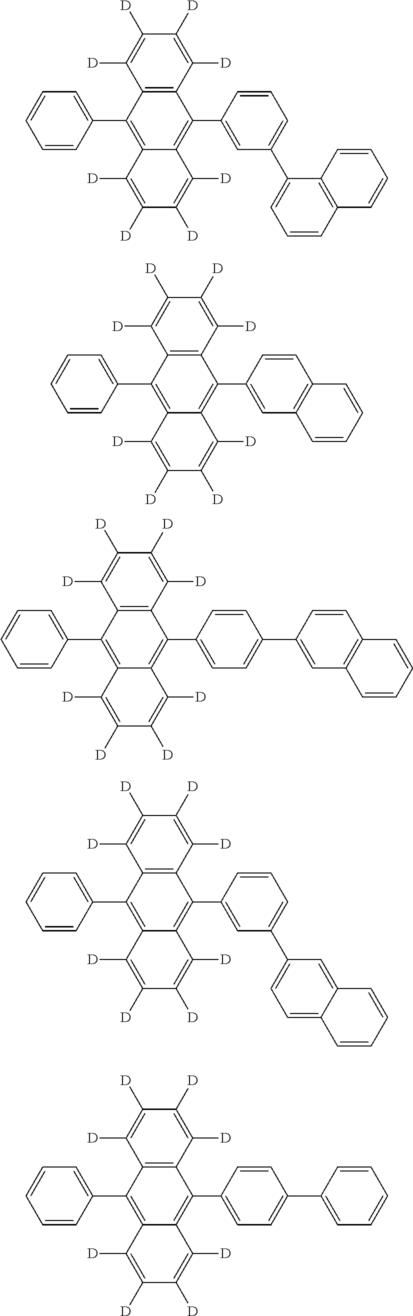

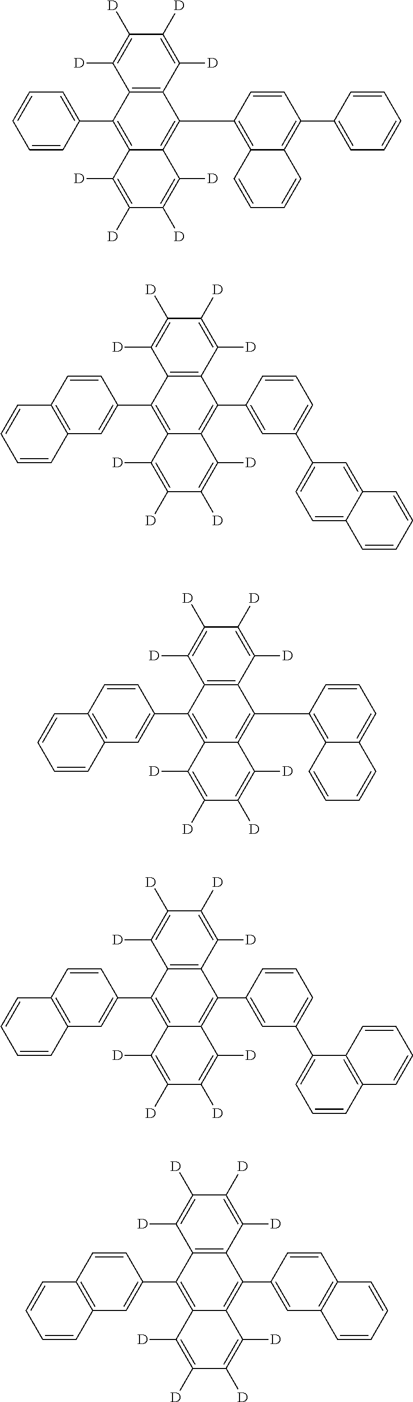

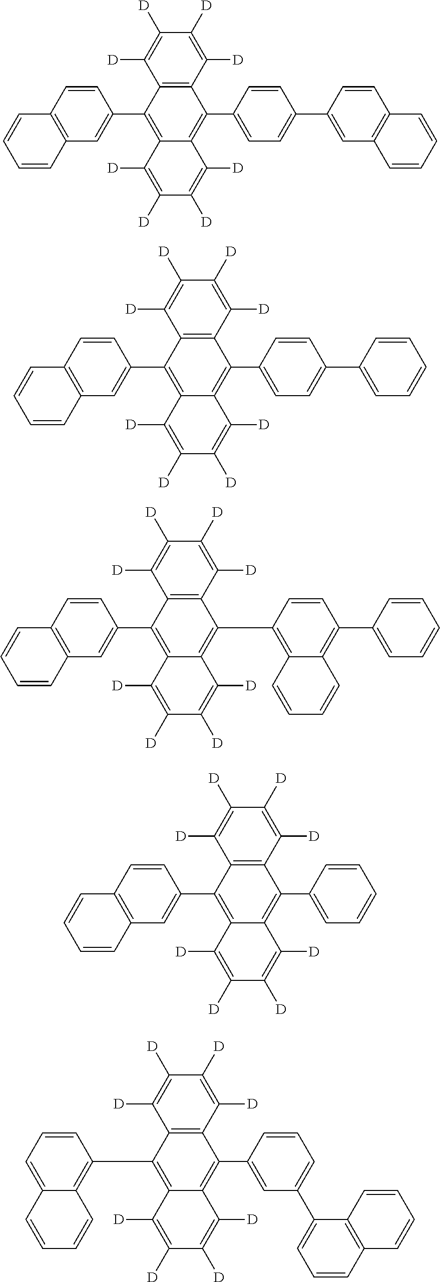

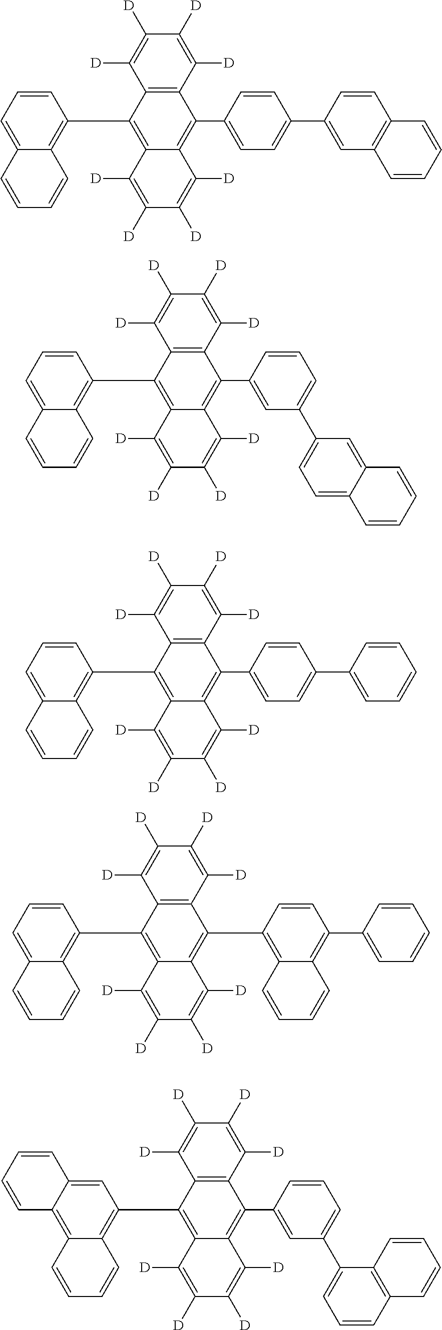

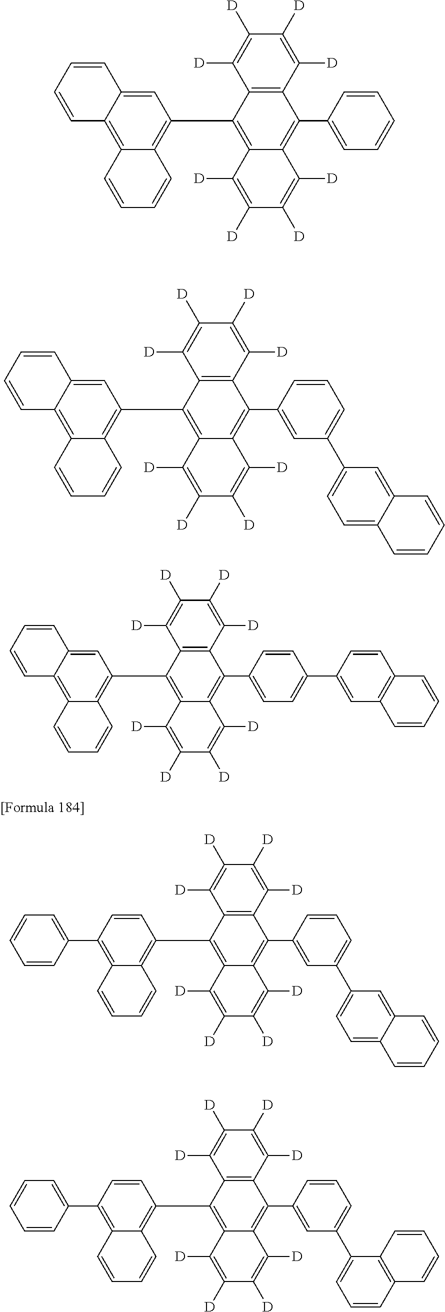

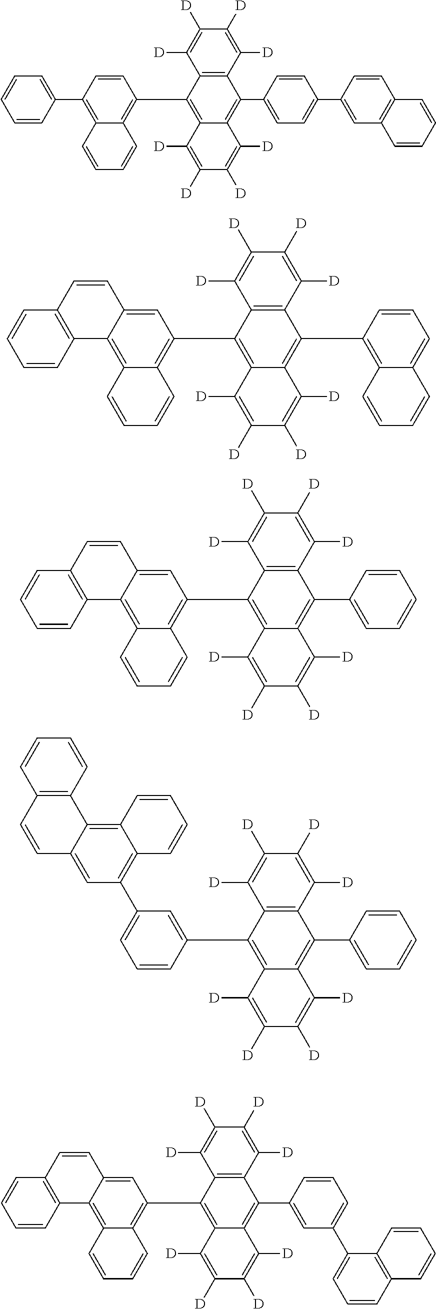

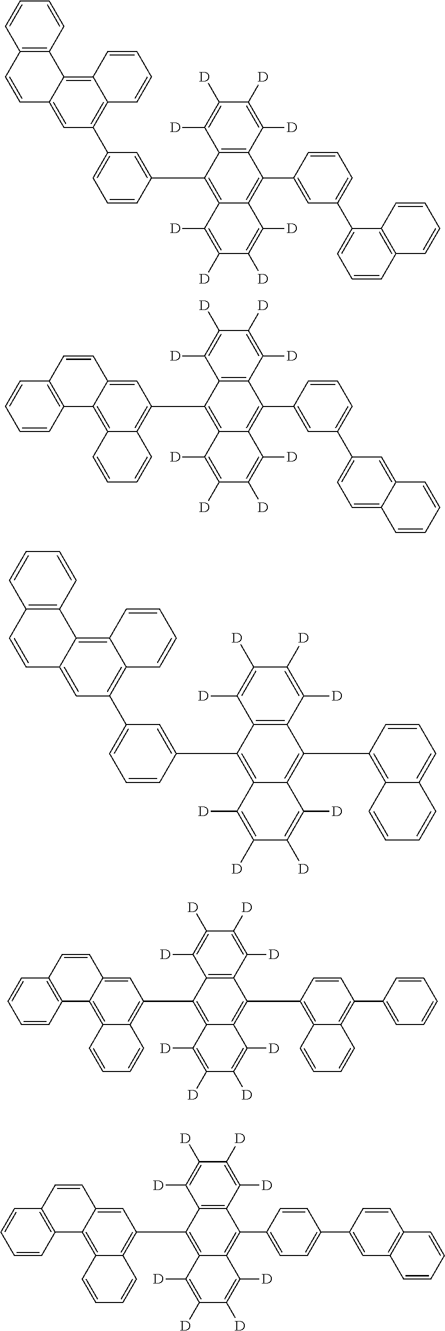

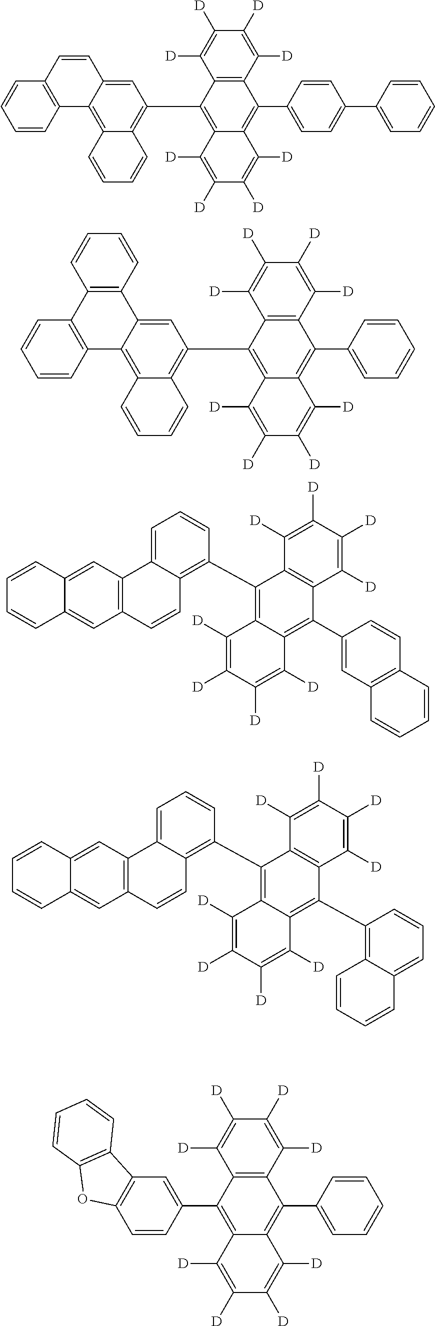

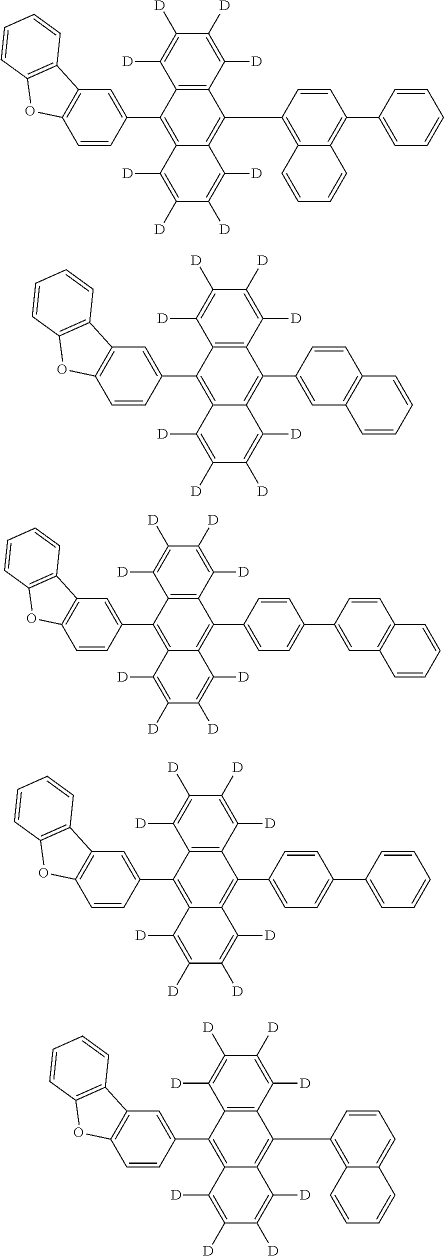

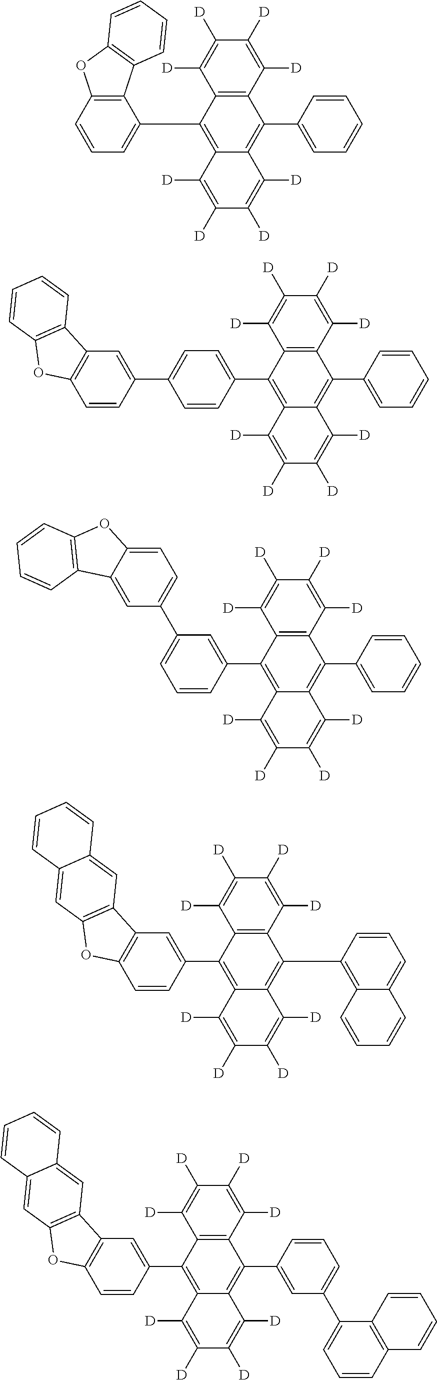

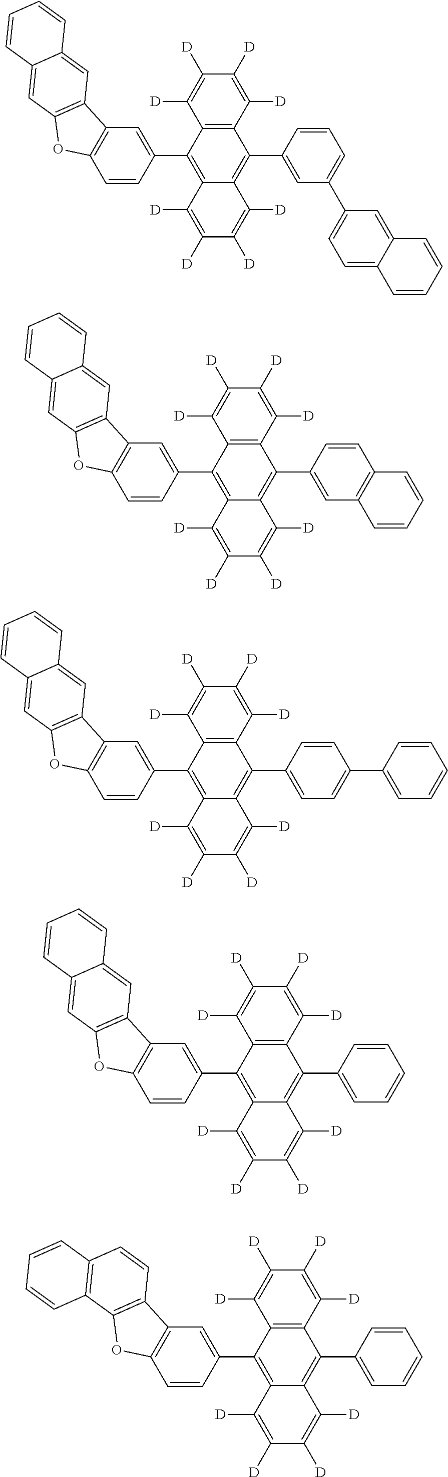

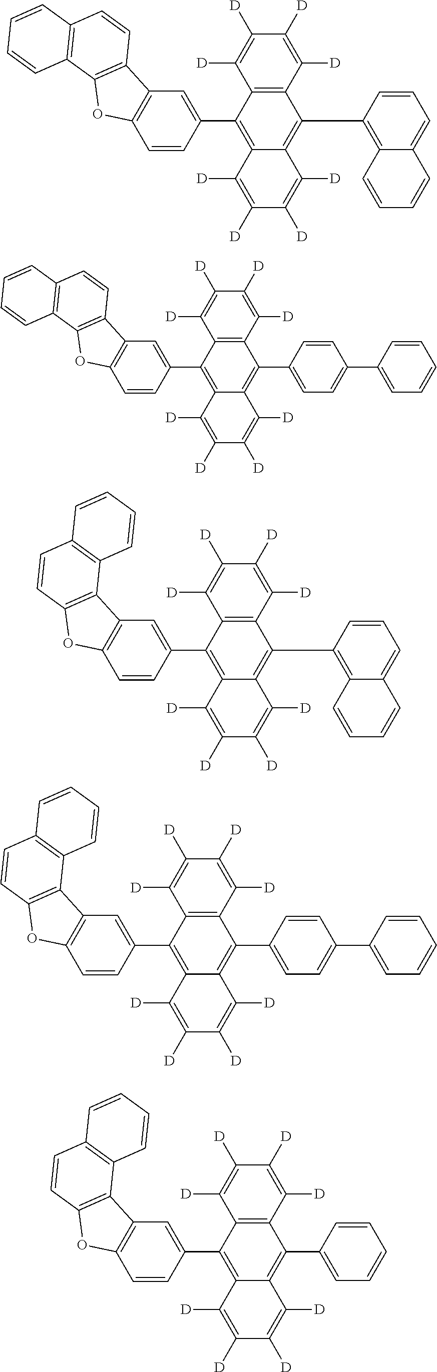

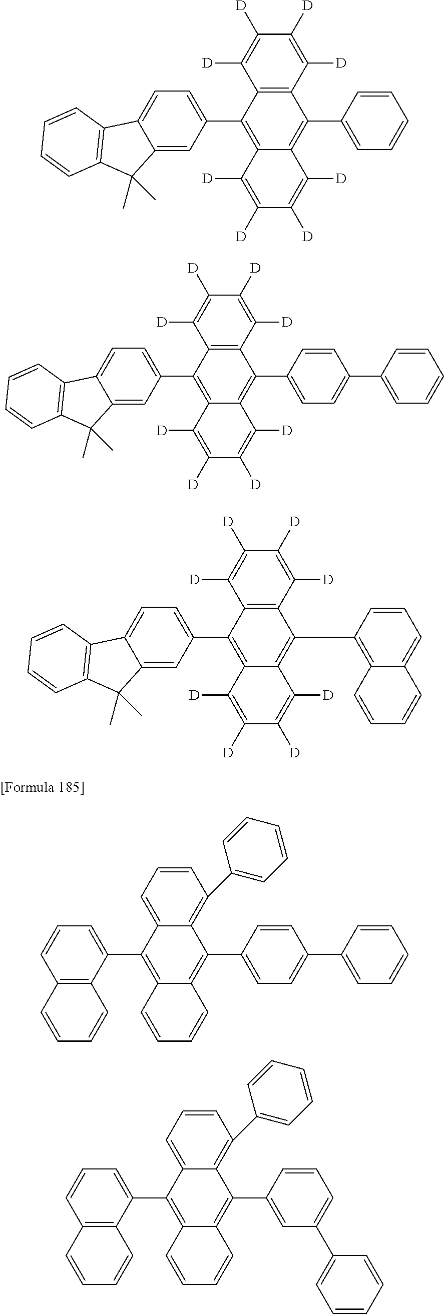

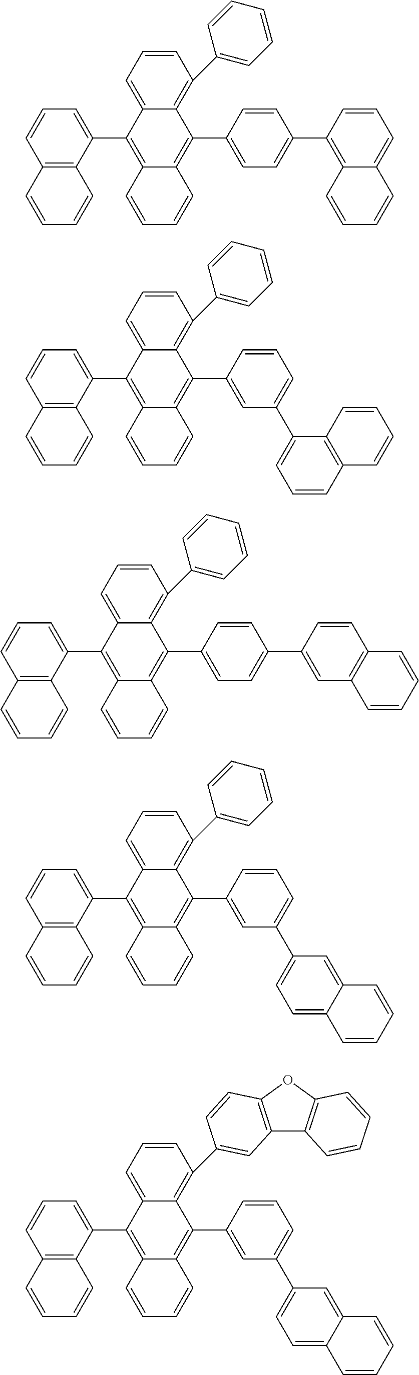

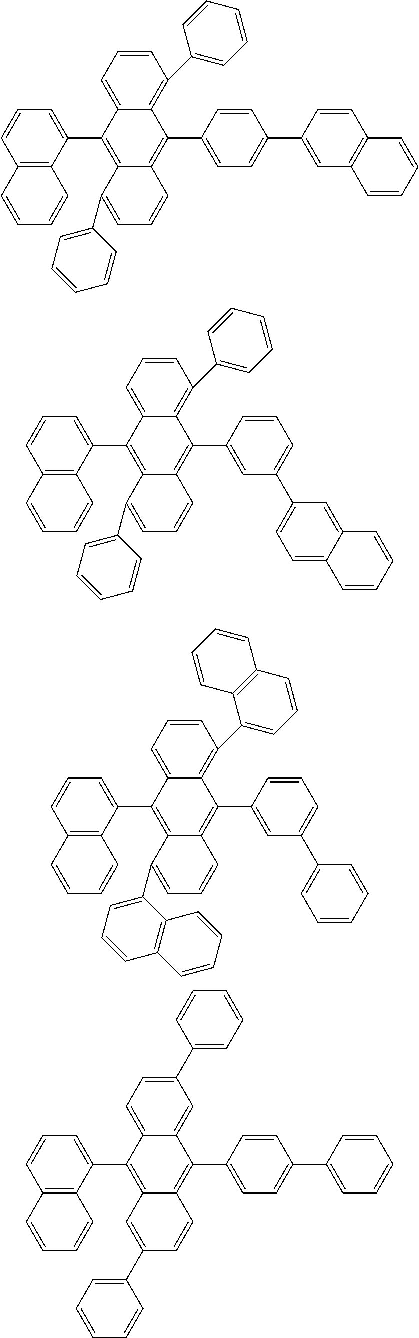

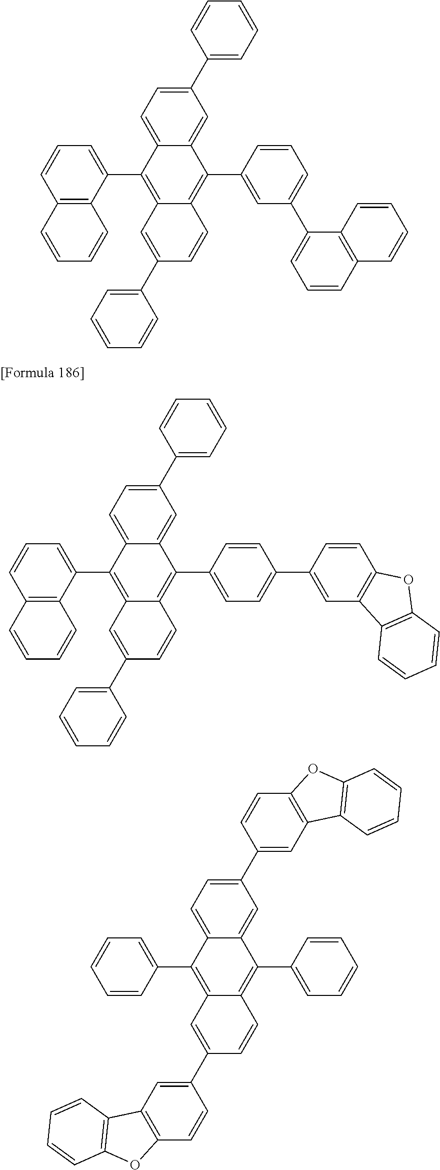

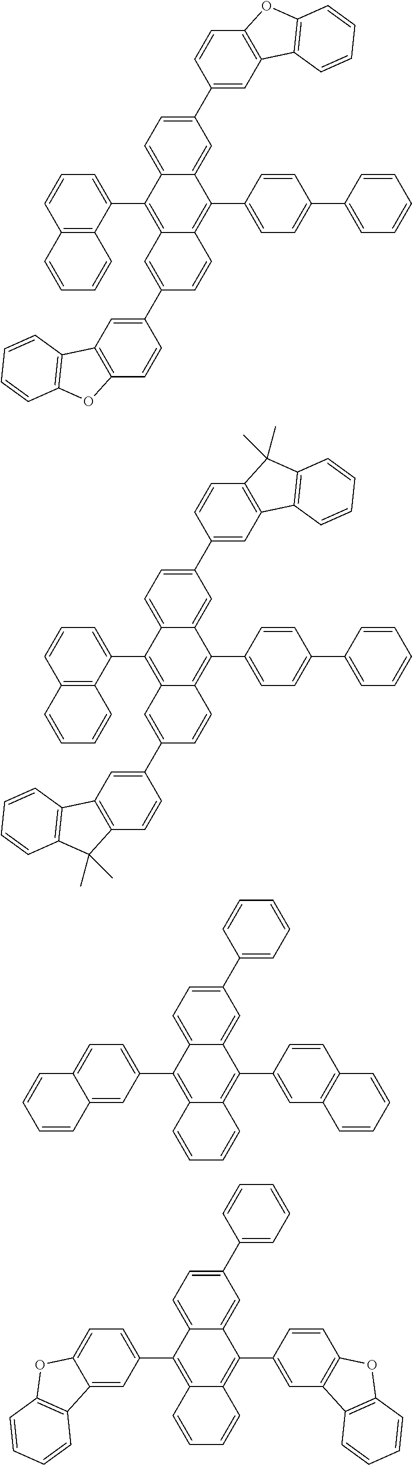

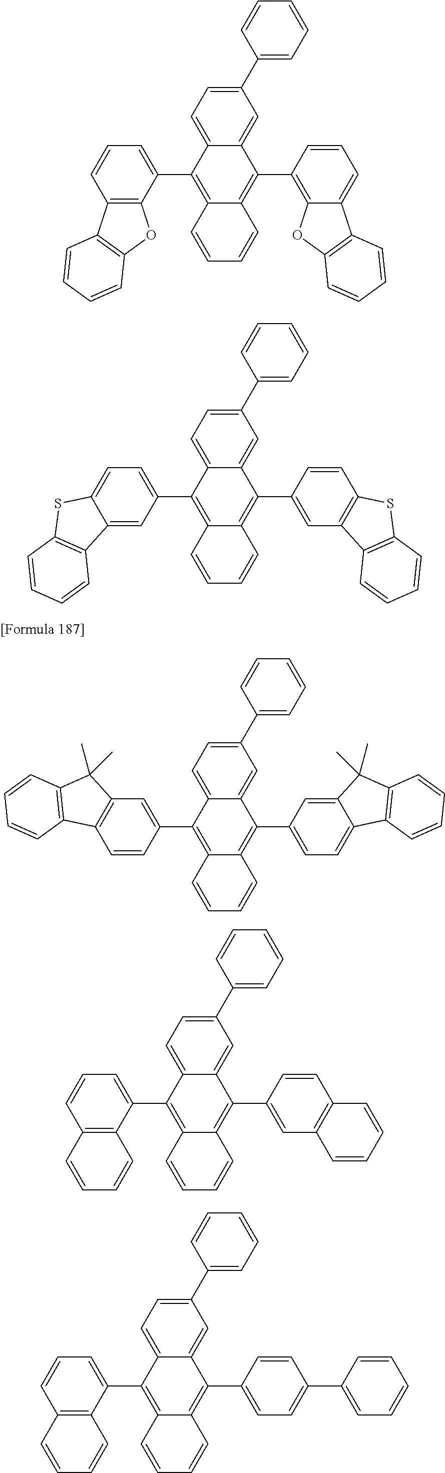

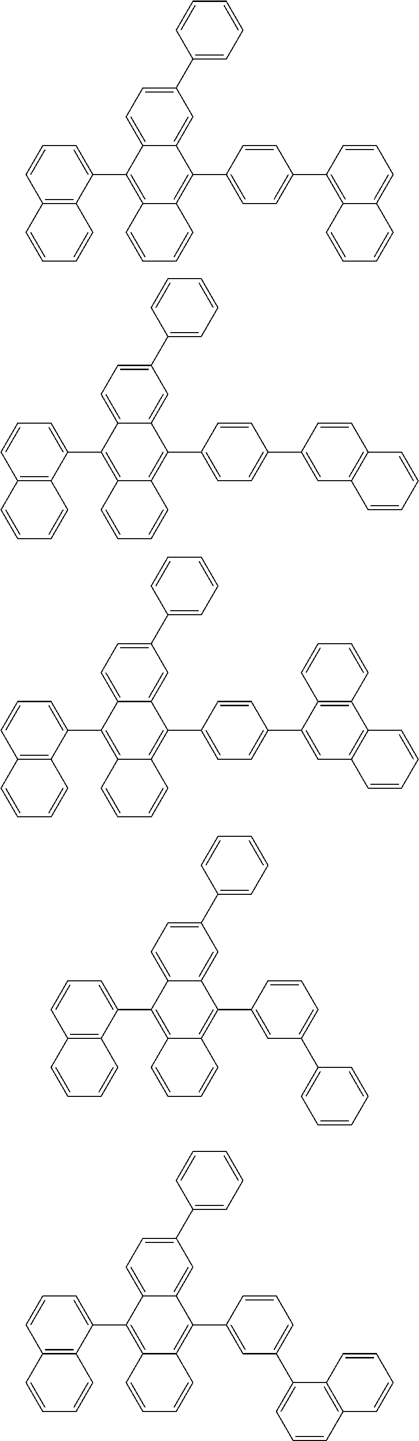

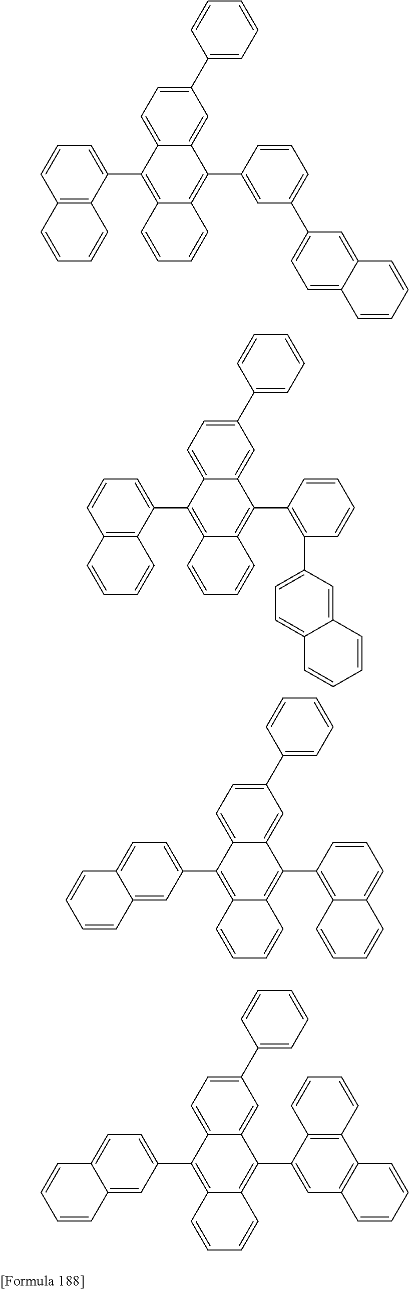

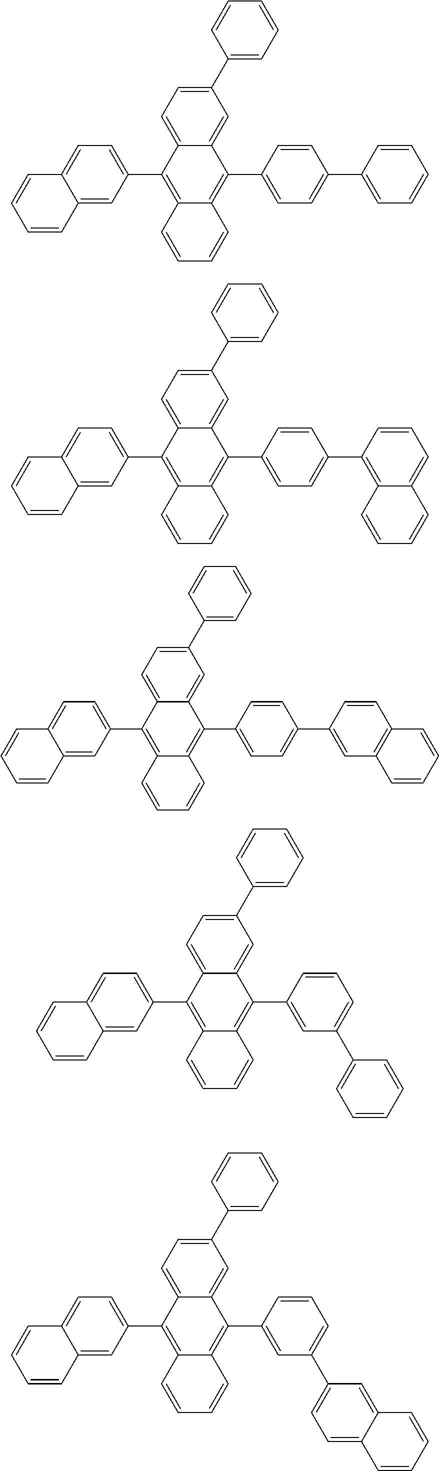

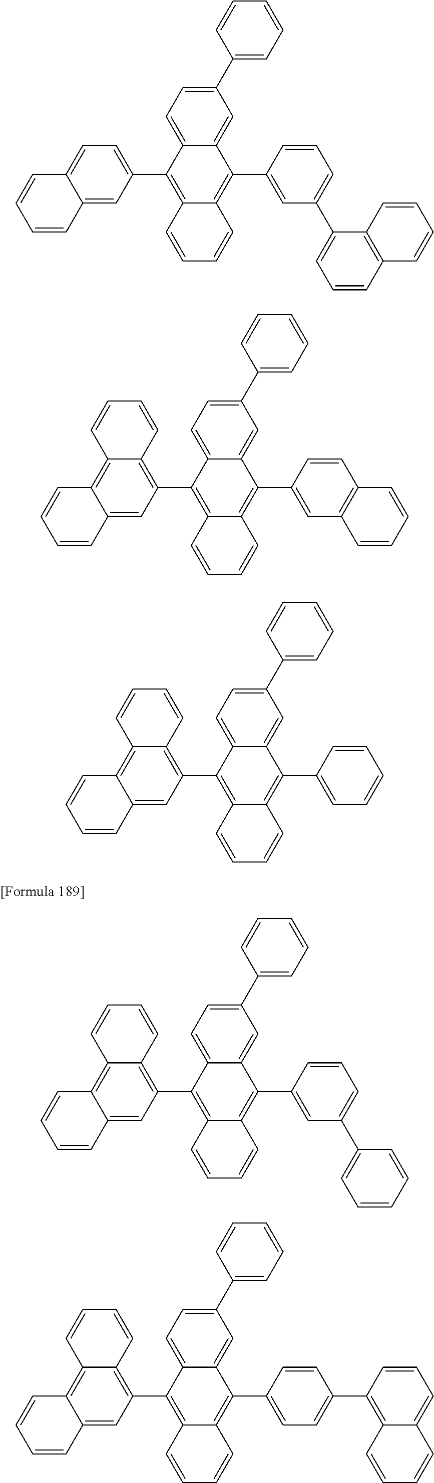

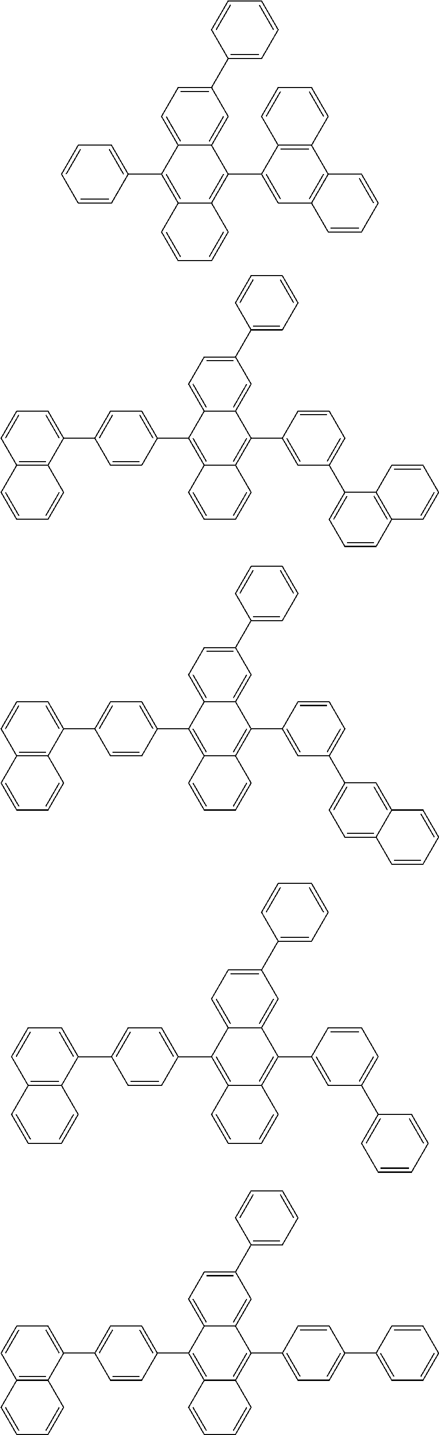

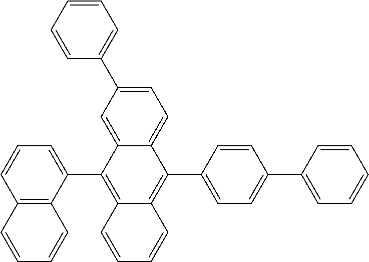 [SMILES: c1ccc(-c2ccc(-c3c4ccccc4c(-c4cccc5ccccc45)c4cc(-c5ccccc5)ccc34)cc2)cc1]

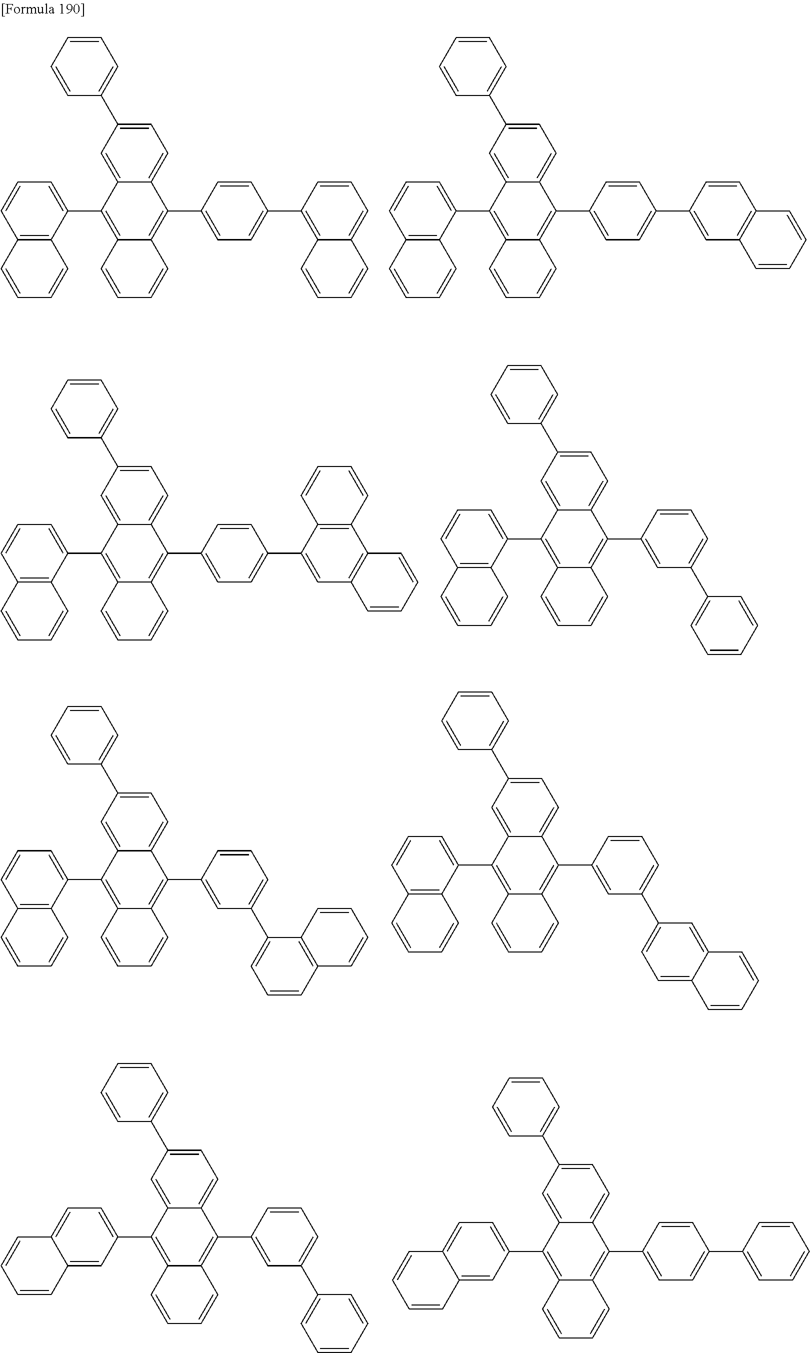

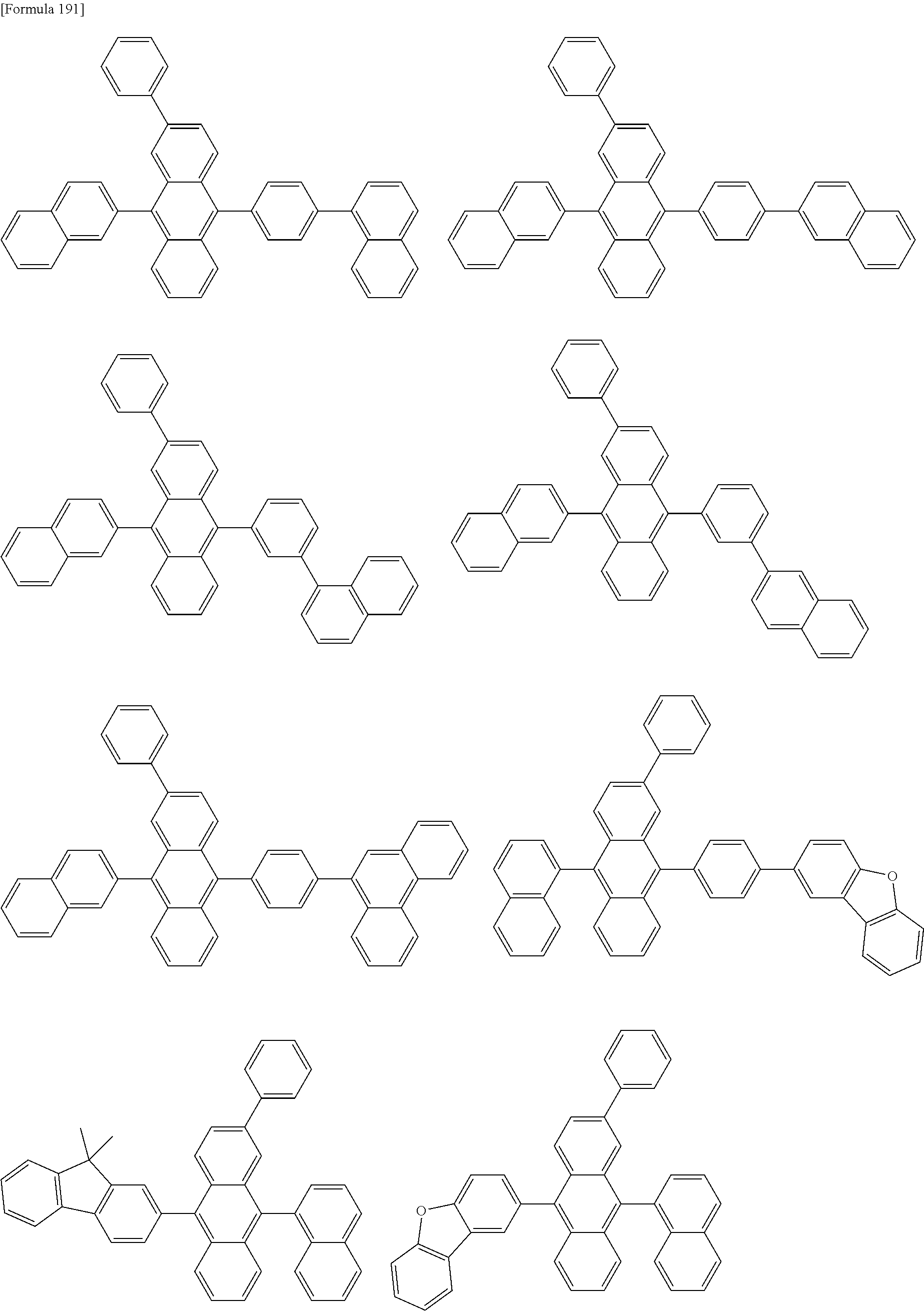

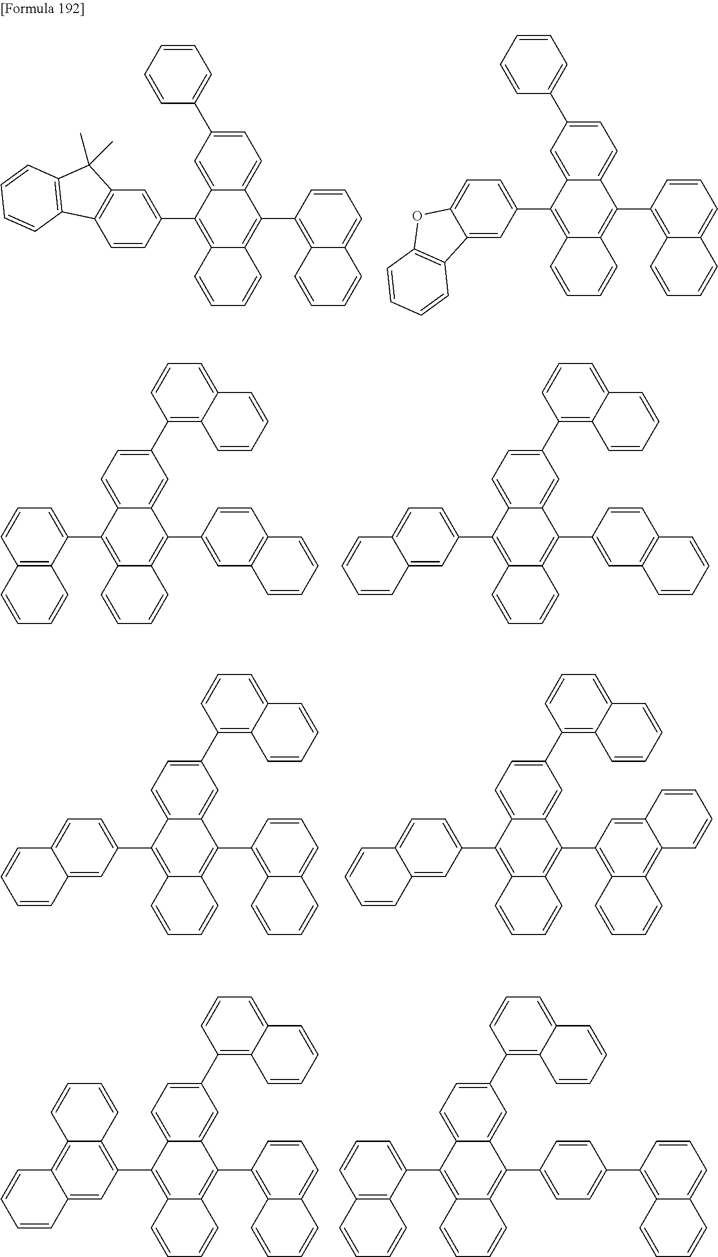

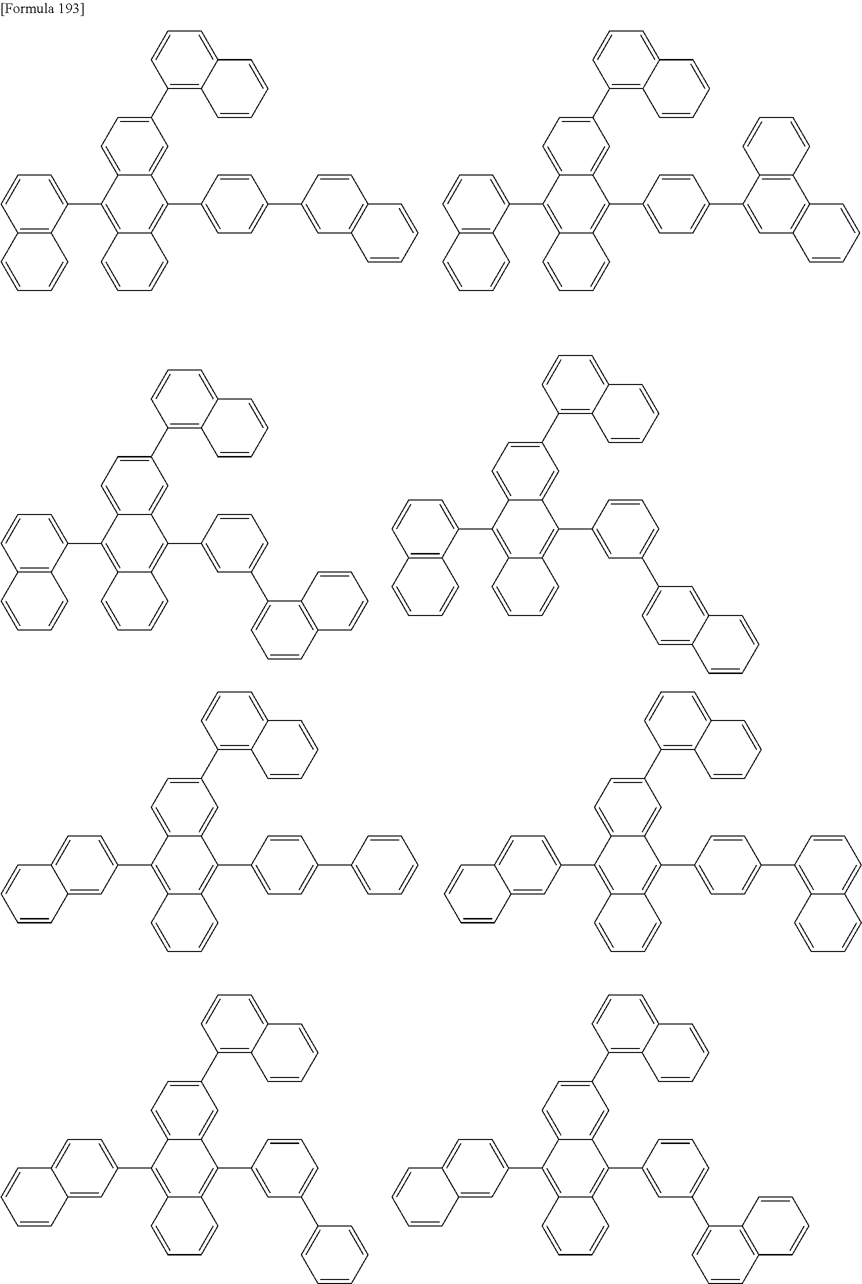

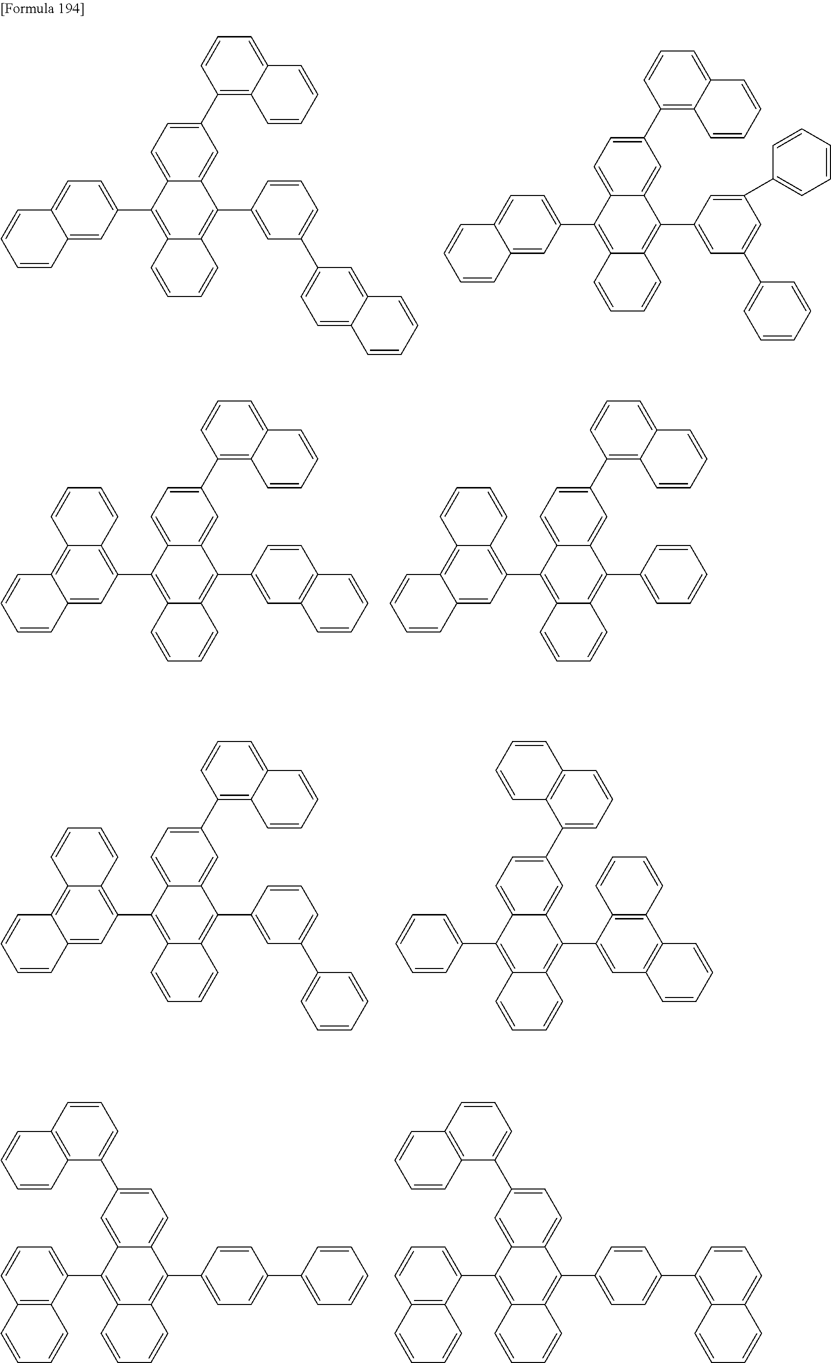

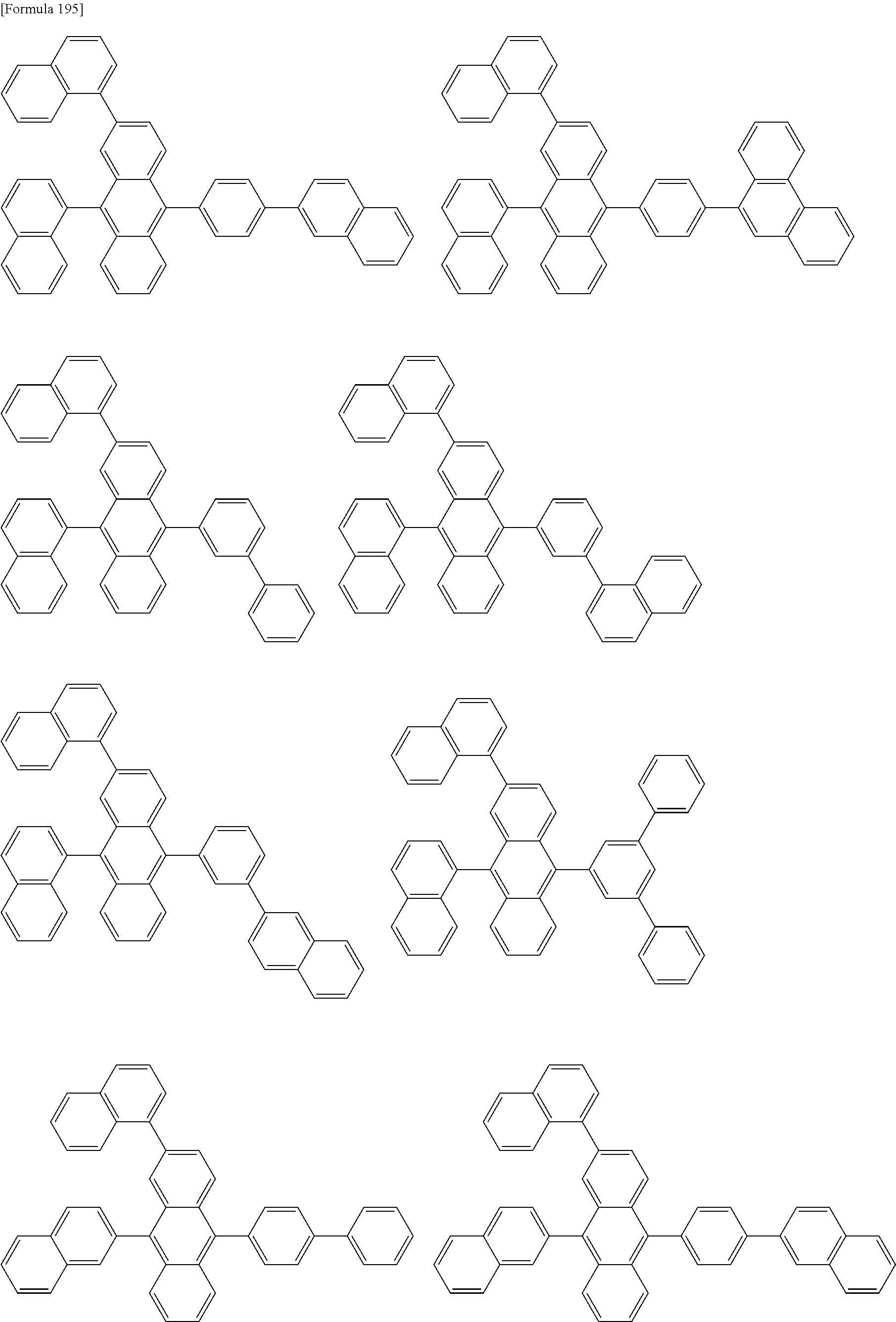

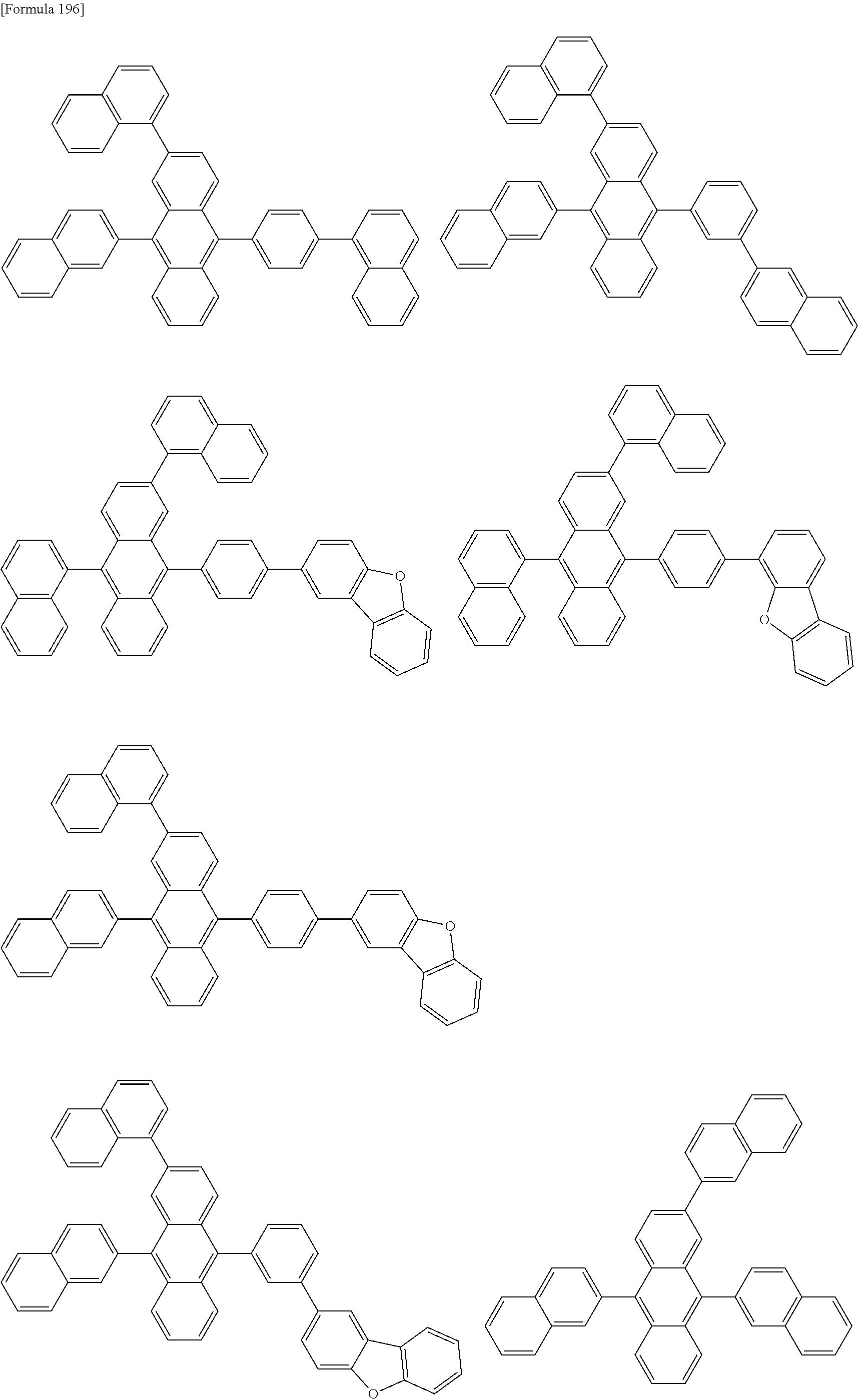

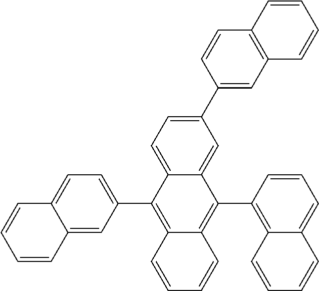 [SMILES: c1ccc2cc(-c3ccc4c(-c5ccc6ccccc6c5)c5ccccc5c(-c5cccc6ccccc56)c4c3)ccc2c1]

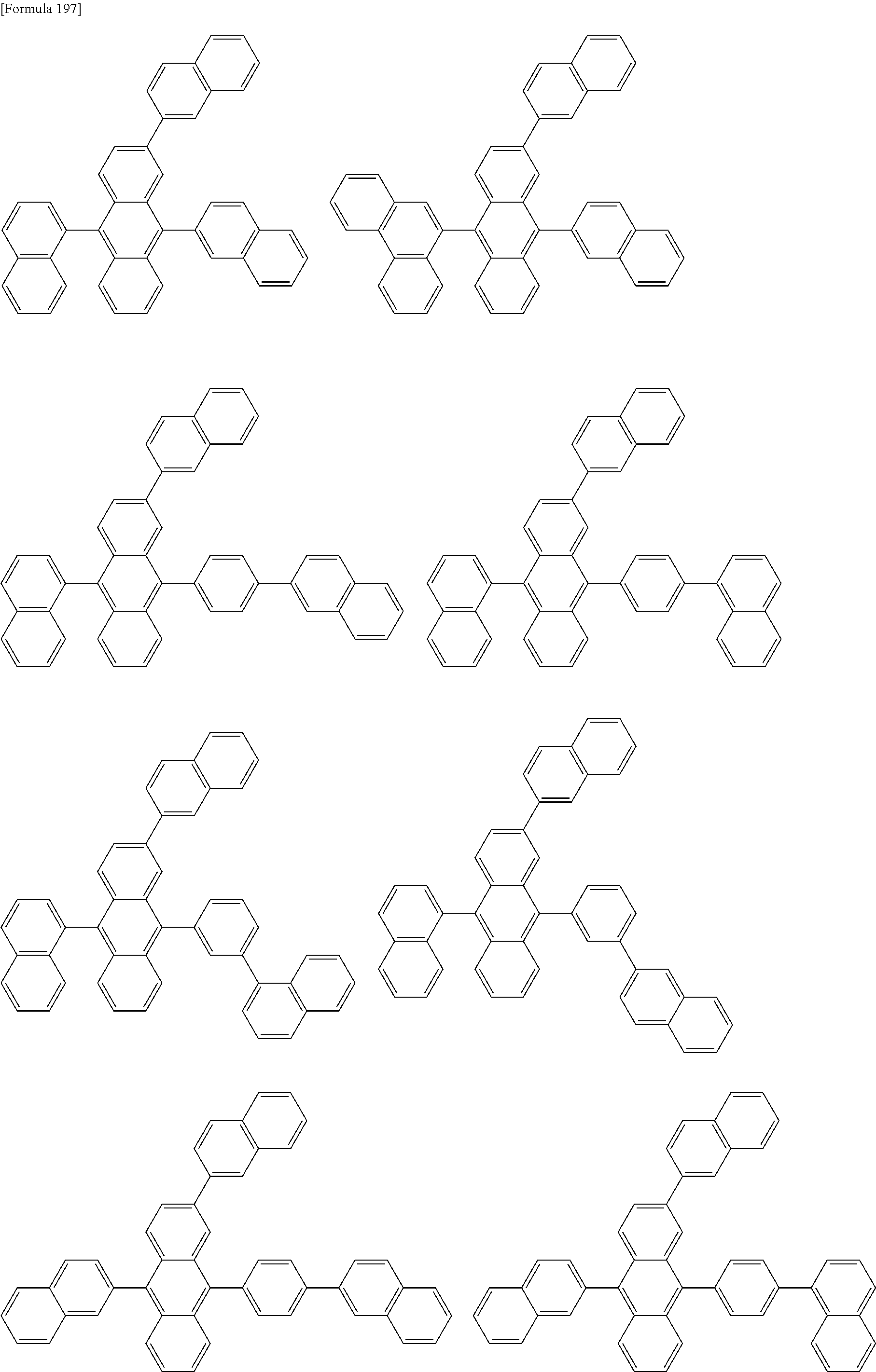

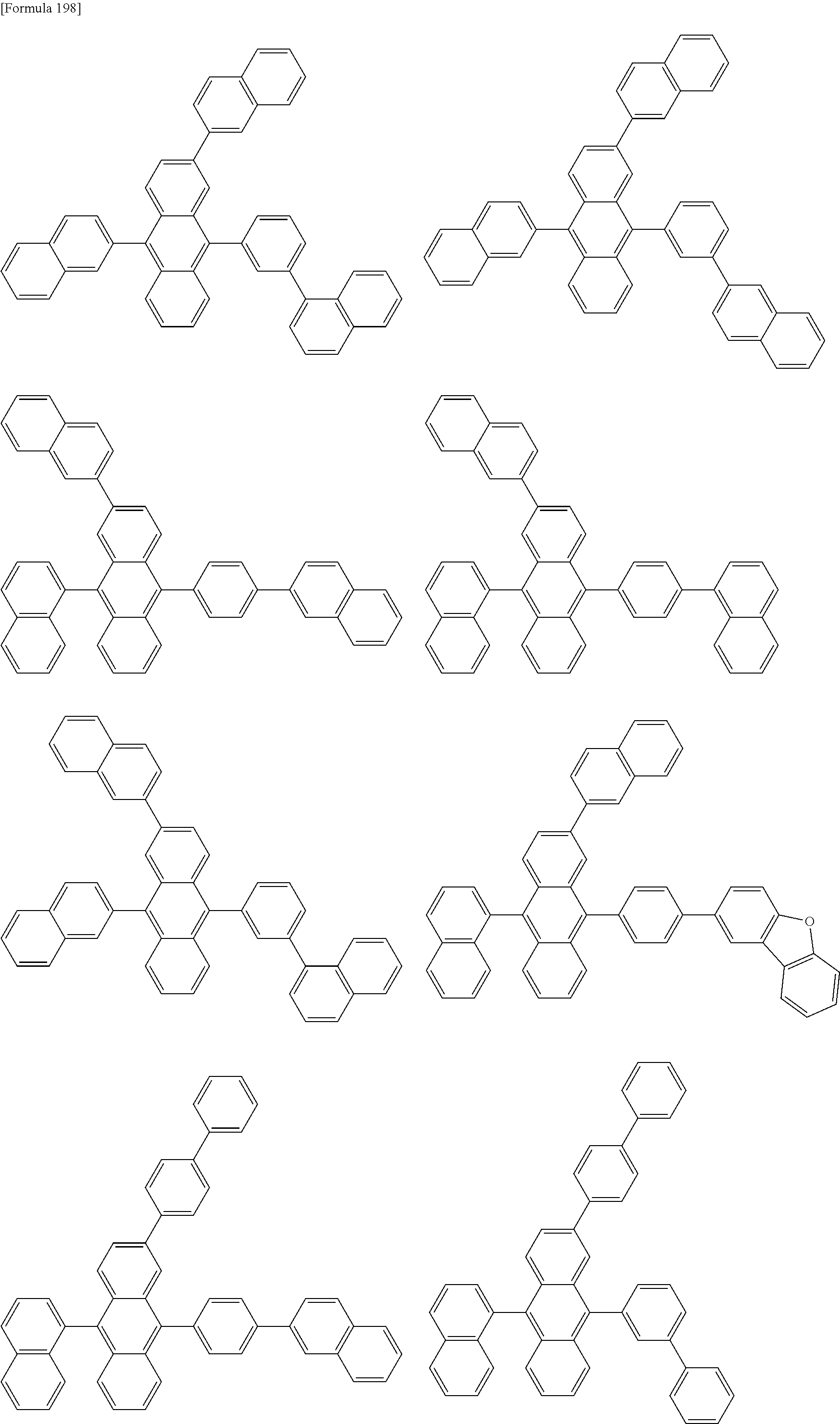

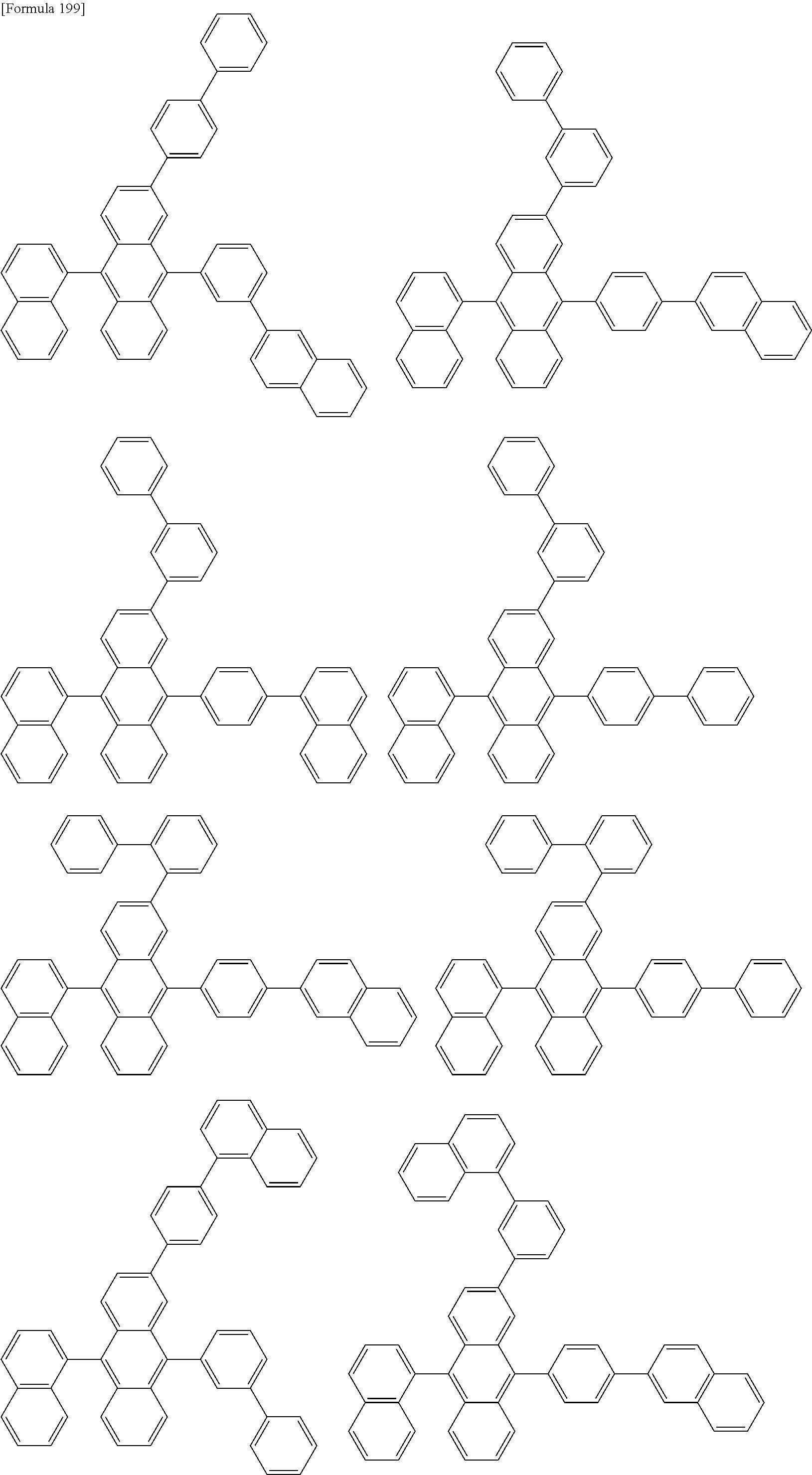

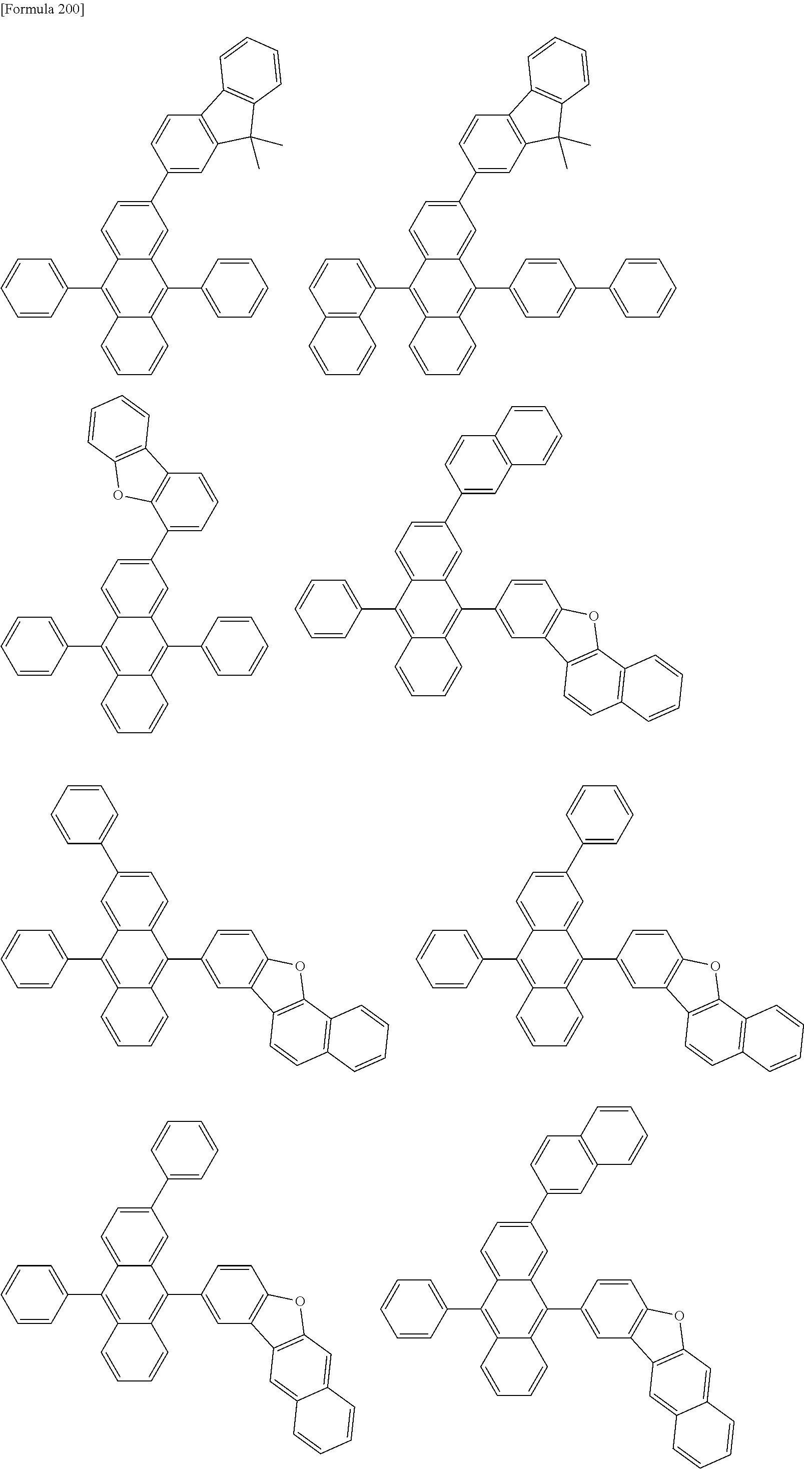

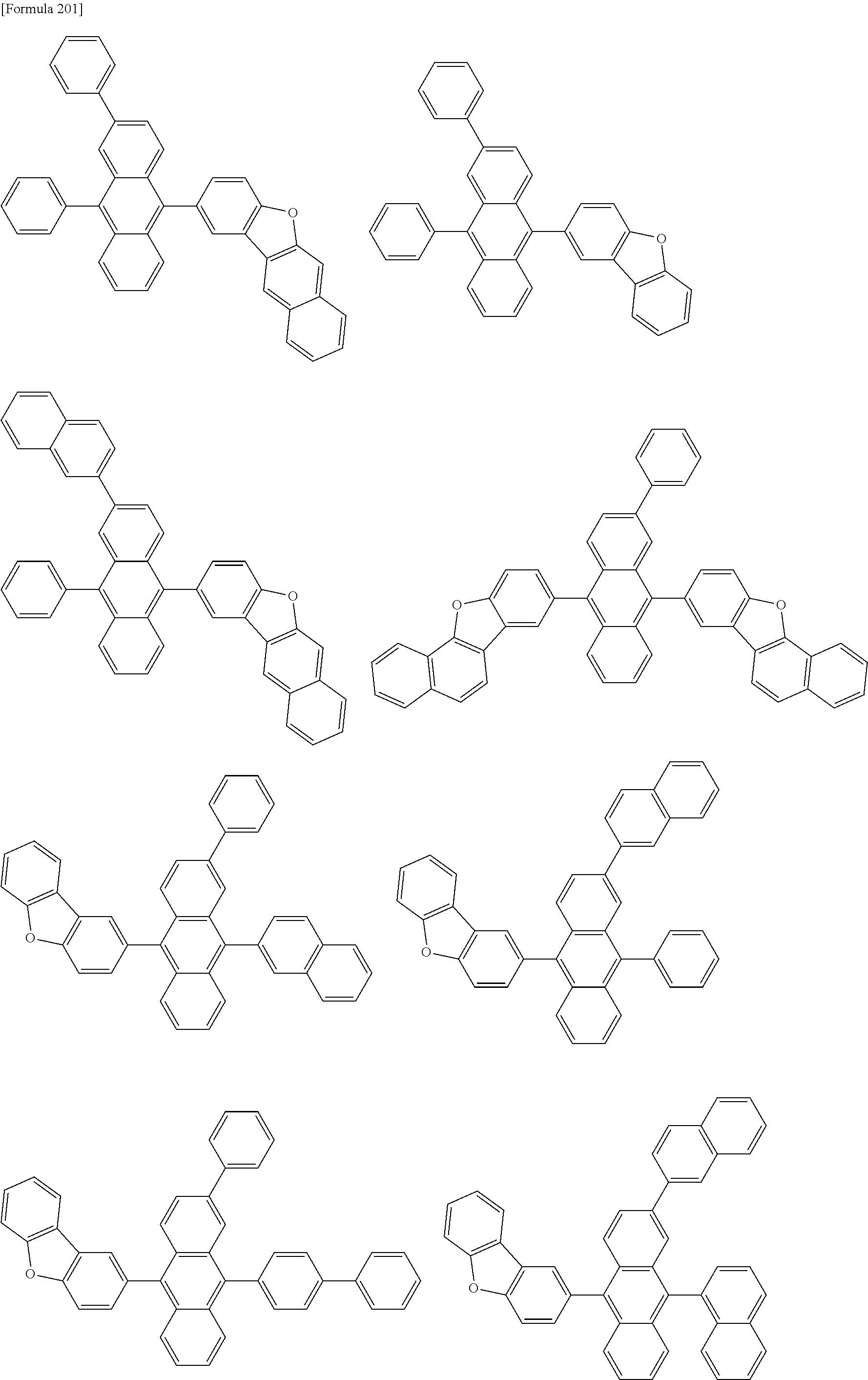

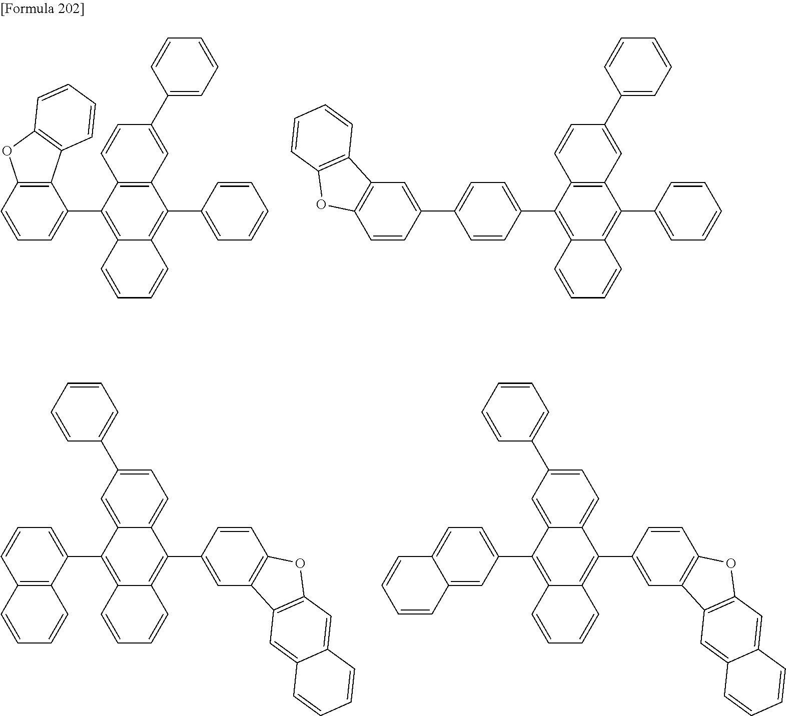 [SMILES: c1ccc(-c2ccc3c(-c4cccc5oc6ccccc6c45)c4ccccc4c(-c4ccccc4)c3c2)cc1 ; c1ccc(-c2ccc3c(-c4ccc(-c5ccc6oc7ccccc7c6c5)cc4)c4ccccc4c(-c4ccccc4)c3c2)cc1 ; c1ccc(-c2ccc3c(-c4ccc5oc6cc7ccccc7cc6c5c4)c4ccccc4c(-c4cccc5ccccc45)c3c2)cc1 ; c1ccc(-c2ccc3c(-c4ccc5oc6cc7ccccc7cc6c5c4)c4ccccc4c(-c4ccc5ccccc5c4)c3c2)cc1]

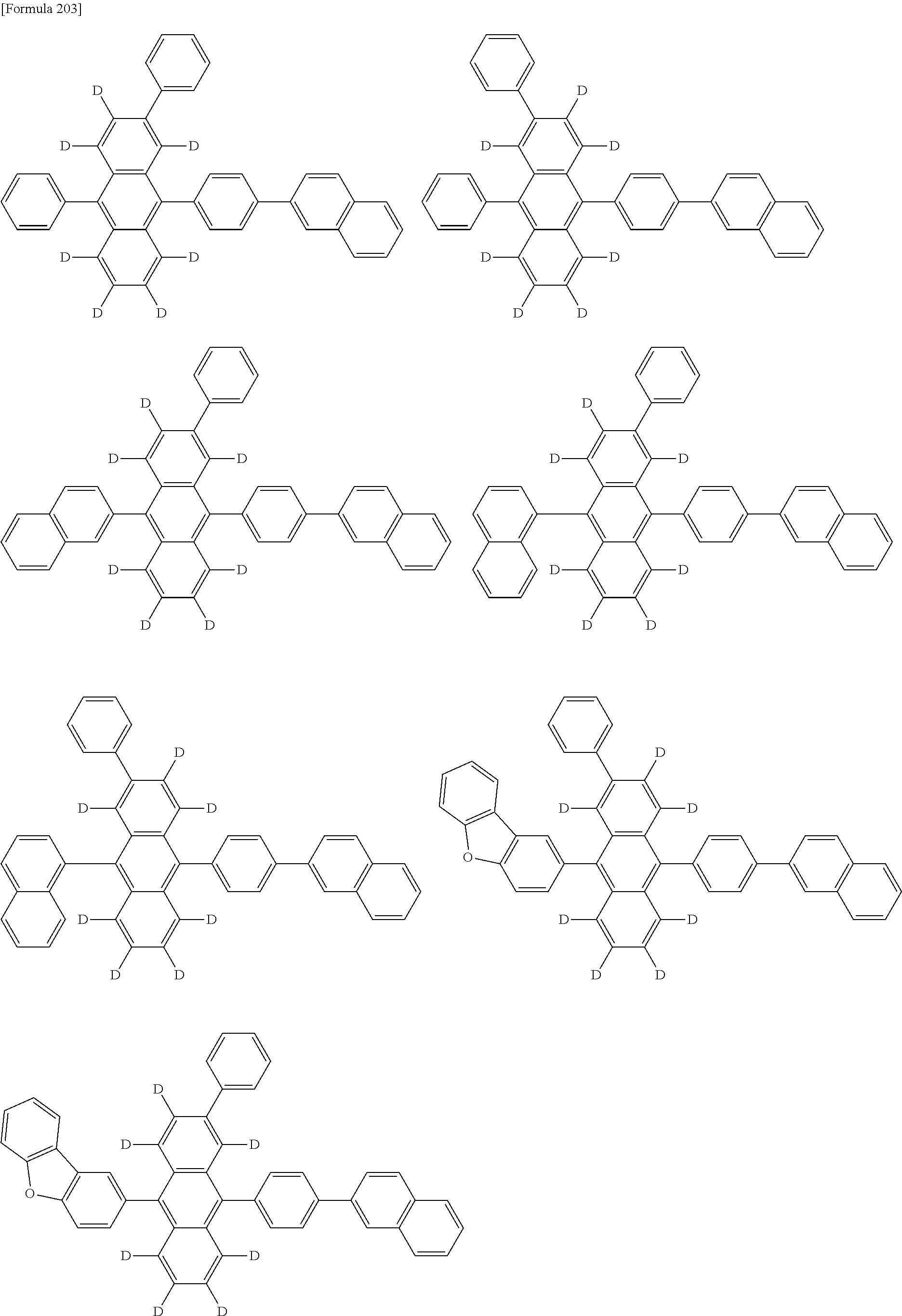

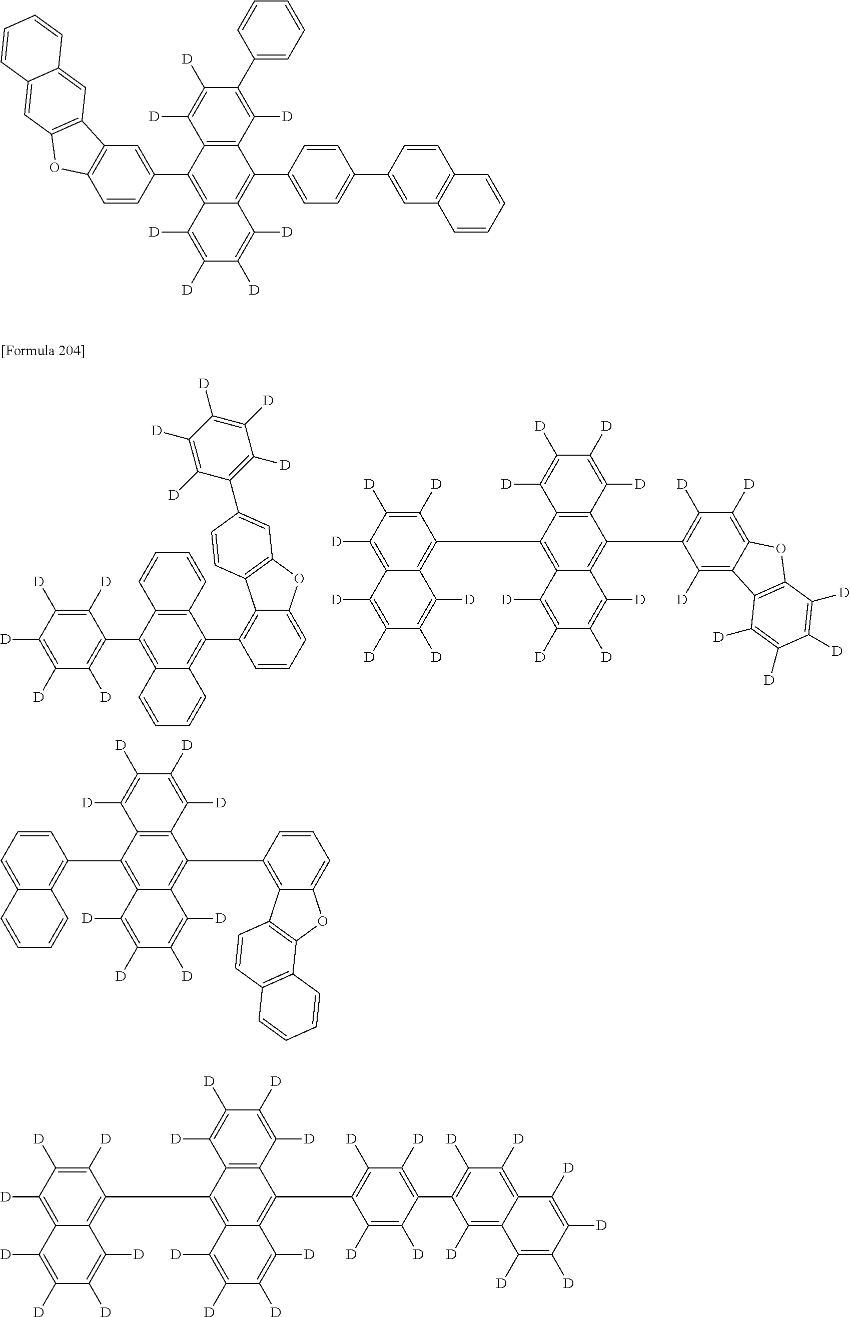

### Luminescent Compound

In an exemplary arrangement of the organic EL device of each of the exemplary embodiments, the luminescent compounds such as the first luminescent compound and the second luminescent compound are not particularly limited. In an exemplary arrangement of the organic EL device of each of the exemplary embodiments, each luminescent compound contains no metal complex.

For instance, the luminescent compounds such as the first luminescent compound and the second luminescent compound are each independently at least one compound selected from the group consisting of a compound represented by a formula (5) and a compound represented by a formula (6) below.

### Compound Represented by Formula (5)

The compound represented by the formula (5) will be described below.

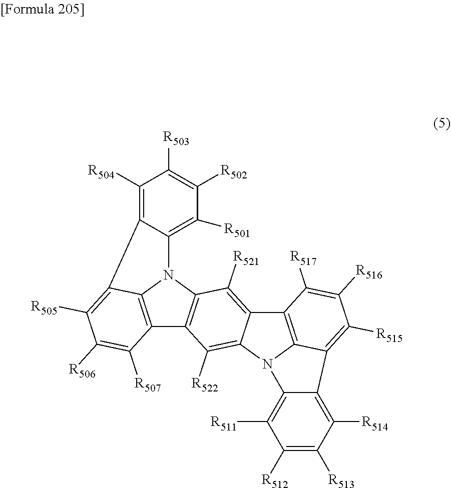

In the formula (5):
at least one combination of adjacent two or more of R₅₀₁ to R₅₀₇ and R₅₁₁ to R₅₁₇ are mutually bonded to form a substituted or unsubstituted monocyclic ring, mutually bonded to form a substituted or unsubstituted fused ring, or not mutually bonded;
R₅₀₁ to R₅₀₇ and R₅₁₁ to R₅₁₇ forming neither the monocyclic ring nor the fused ring are each independently a hydrogen atom, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a halogen atom, a cyano group, a nitro group, a substituted or unsubstituted aryl group having 6 to 50 ring carbon atoms, or a substituted or unsubstituted heterocyclic group having 5 to 50 ring atoms; and
R₅₂₁ and R₅₂₂ are each independently a hydrogen atom, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a halogen atom, a cyano group, a nitro group, a substituted or unsubstituted aryl group having 6 to 50 ring carbon atoms, or a substituted or unsubstituted heterocyclic group having 5 to 50 ring atoms.

In the luminescent compounds such as the first luminescent compound and the second luminescent compound, R₉₀₁, R₉₀₂, R₉₀₃, R₉₀₄, R₉₀₅, R₉₀₆, R₉₀₇, R₈₀₁ and R₈₀₂ are each independently a hydrogen atom, a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms;
when a plurality of R₉₀₁ are present, the plurality of R₉₀₁ are mutually the same or different;
when a plurality of R₉₀₂ are present, the plurality of R₉₀₂ are mutually the same or different;
when a plurality of Roos are present, the plurality of Roos are mutually the same or different;
when a plurality of R₉₀₄ are present, the plurality of R₉₀₄ are mutually the same or different;
when a plurality of R₉₀₅ are present, the plurality of R₉₀₅ are mutually the same or different;
when a plurality of R₉₀₆ are present, the plurality of R₉₀₆ are mutually the same or different;
when a plurality of R₉₀₇ are present, the plurality of R₉₀₇ are mutually the same or different;
when a plurality of R₈₀₁ are present, the plurality of R₈₀₁ are mutually the same or different; and
when a plurality of R₈₀₂ are present, the plurality of R₈₀₂ are mutually the same or different.

“A combination of adjacent two or more of R₅₀₁ to R₅₀₇ and R₅₁₁ to R₅₁₇” refers to, for instance, a combination of R₅₀₁ and R₅₀₂, a combination of R₅₀₂ and R₅₀₃, a combination of R₅₀₃ and R₅₀₄, a combination of R₅₀₅ and R₅₀₆, a combination of R₅₀₆ and R₅₀₇, and a combination of R₅₀₁, R₅₀₂, and R₅₀₃.

In an exemplary embodiment, at least one, preferably two, selected from the group consisting of R₅₀₁ to R₅₀₇ and R₅₁₁ to R₅₁₇ are each a group represented by —N(R₉₀₆)(R₉₀₇).

In an exemplary embodiment, R₅₀₁ to R₅₀₇ and R₅₁₁ to R₅₁₇ are each independently a hydrogen atom, a substituted or unsubstituted aryl group having 6 to 50 ring carbon atoms, or a substituted or unsubstituted heterocyclic group having 5 to 50 ring atoms.

In an exemplary embodiment, the compound represented by the formula (5) is a compound represented by a formula (52) below.

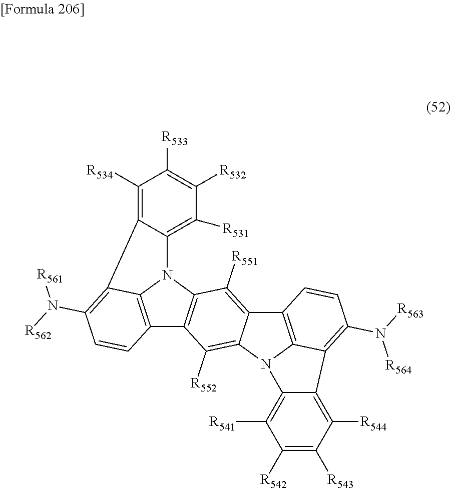

In the formula (52):
at least one combination of adjacent two or more of R₅₃₁ to R₅₃₄ and R₅₄₁ to R₅₄₄ are mutually bonded to form a substituted or unsubstituted monocyclic ring, mutually bonded to form a substituted or unsubstituted fused ring, or not mutually bonded;
R₅₃₁ to R₅₃₄ and R₅₄₁ to R₅₄₄ forming neither the monocyclic ring nor the fused ring, R₅₅₁, and R₅₅₂ are each independently a hydrogen atom, a substituted or unsubstituted aryl group having 6 to 50 ring carbon atoms, or a substituted or unsubstituted heterocyclic group having 5 to 50 ring atoms; and
R₅₆₁ to R₅₆₄ are each independently a substituted or unsubstituted aryl group having 6 to 50 ring carbon atoms, or a substituted or unsubstituted heterocyclic group having 5 to 50 ring atoms.

In an exemplary embodiment, the compound represented by the formula (5) is a compound represented by a formula (53) below.

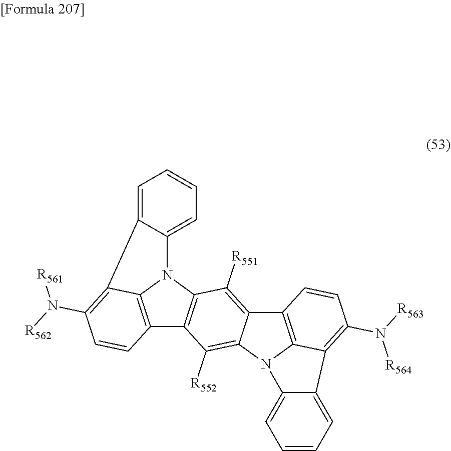

In the formula (53), R₅₅₁, R₅₅₂ and R₅₆₁ to R₅₆₄ each independently represent the same as R₅₅₁, R₅₅₂ and R₅₆₁ to R₅₆₄ in the formula (52).

In an exemplary embodiment, R₅₆₁ to R₅₆₄ in the formulae (52) and (53) are each independently a substituted or unsubstituted aryl group having 6 to 50 ring carbon atoms (preferably a phenyl group).

In an exemplary embodiment, R₅₂₁ and R₅₂₂ in the formula (5) and R₅₅₁ and R₅₅₂ in the formulae (52) and (53) are each a hydrogen atom.

In an exemplary embodiment, the substituent for the “substituted or unsubstituted” group in the formulae (5), (52) and (53) is a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms.

### Specific Examples of Compound Represented by Formula (5)

Specific examples of the compound represented by the formula (5) include compounds as below.

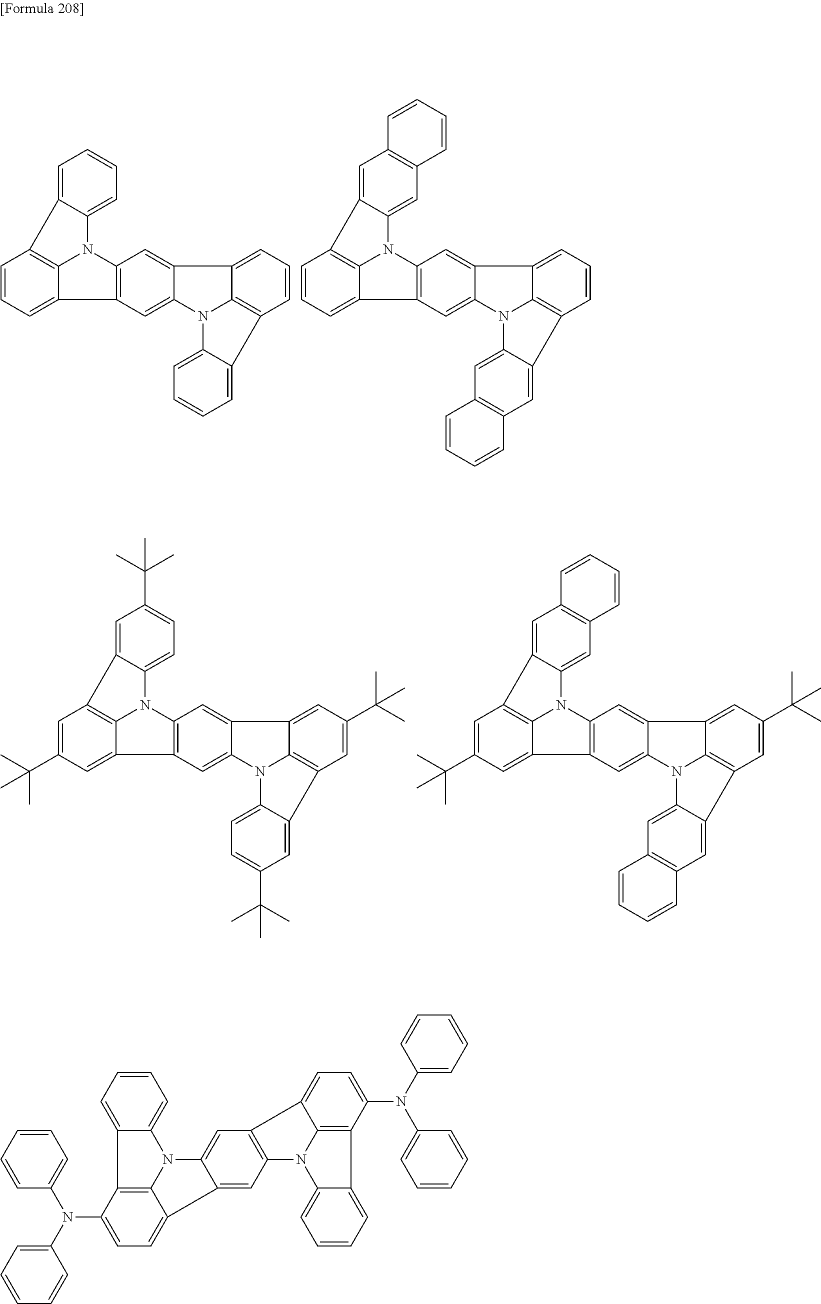

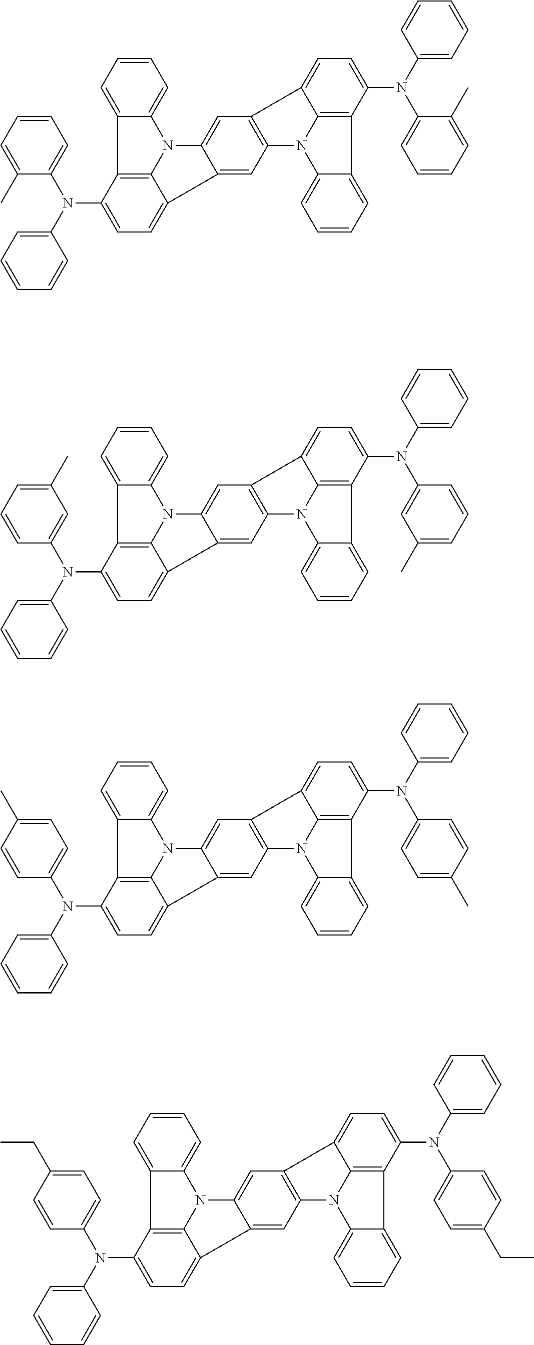

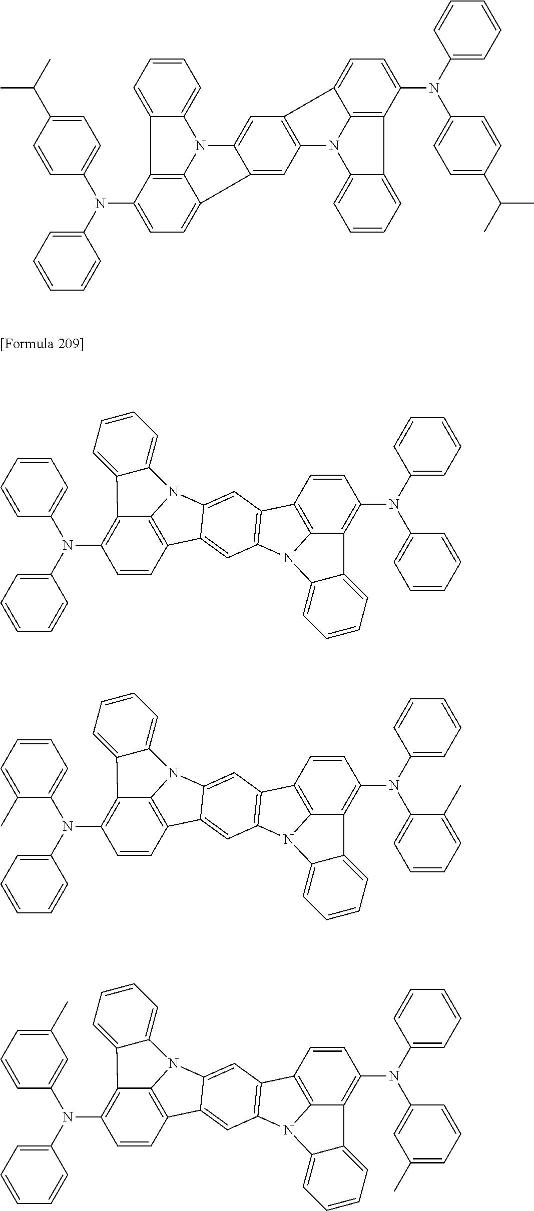

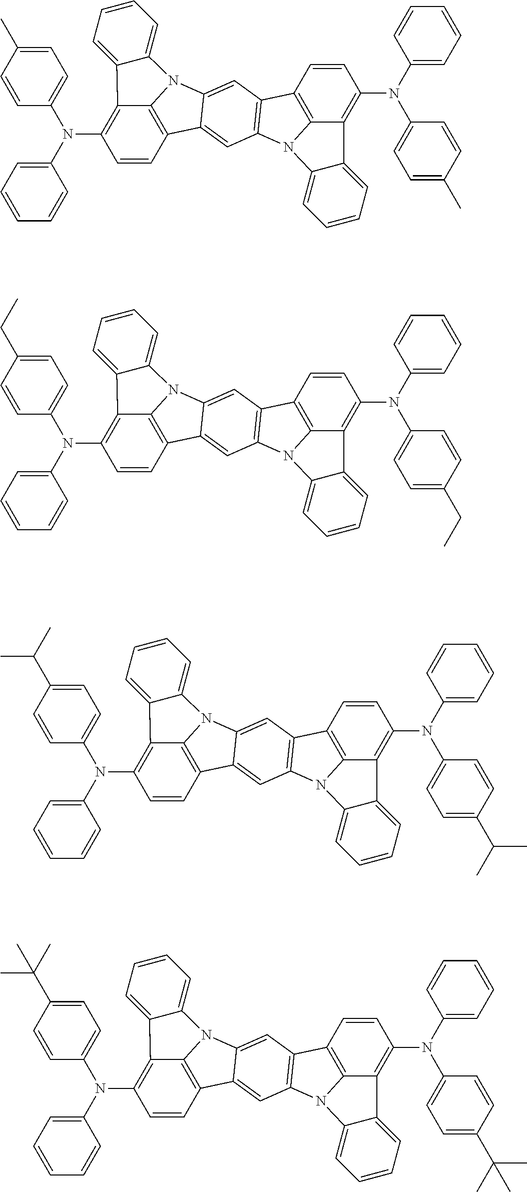

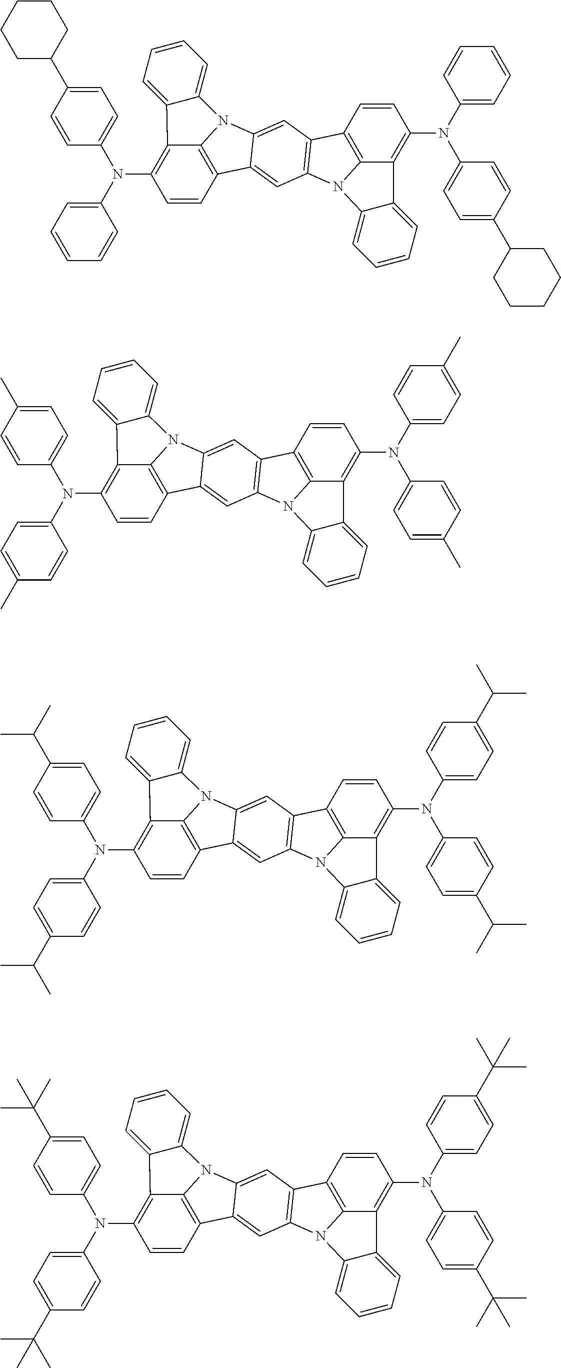

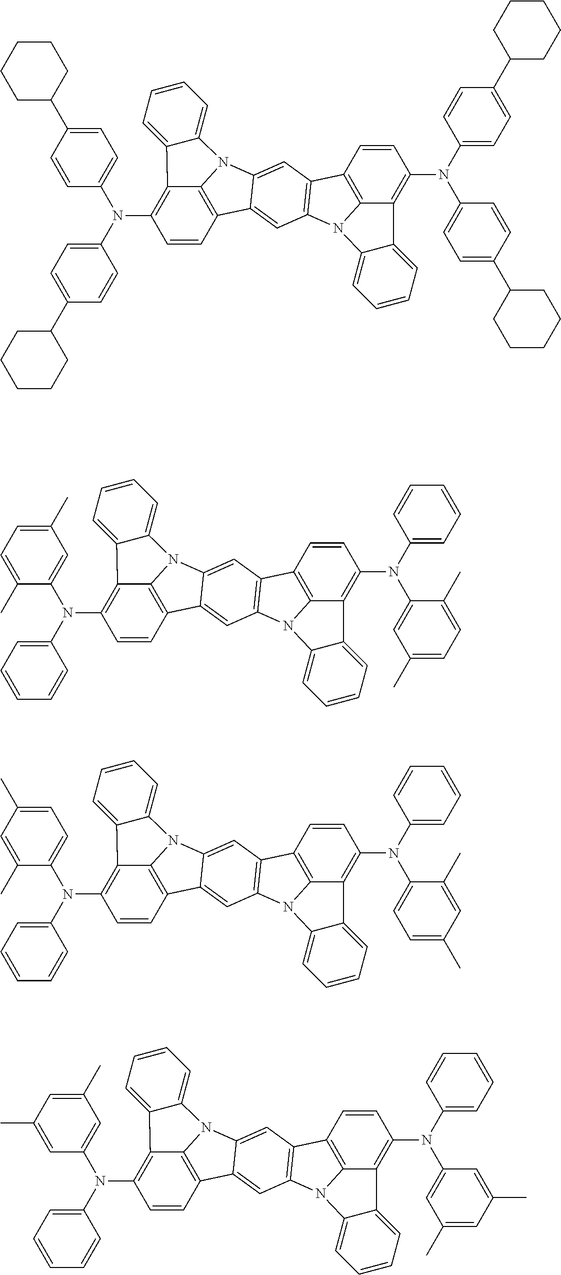

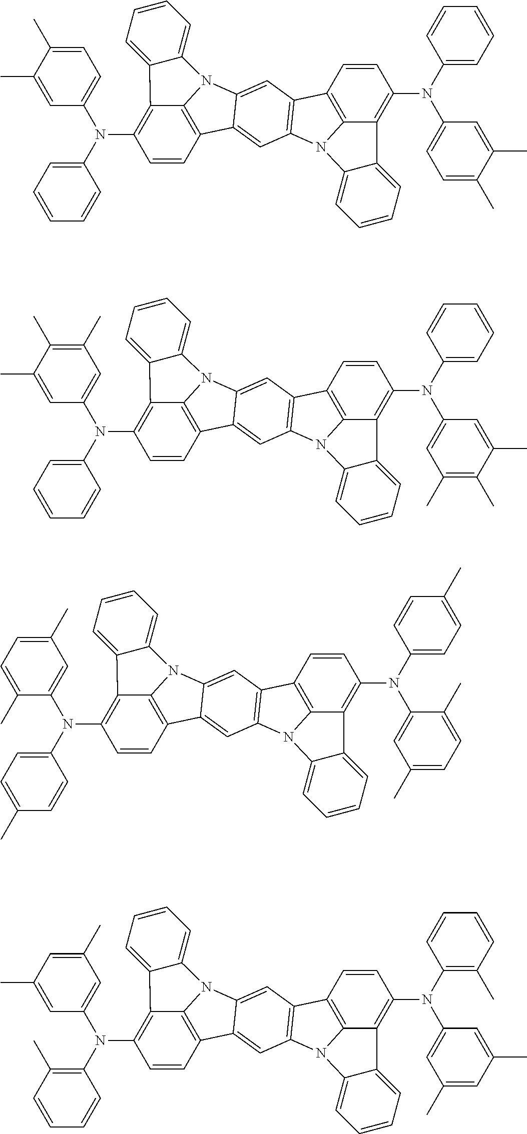

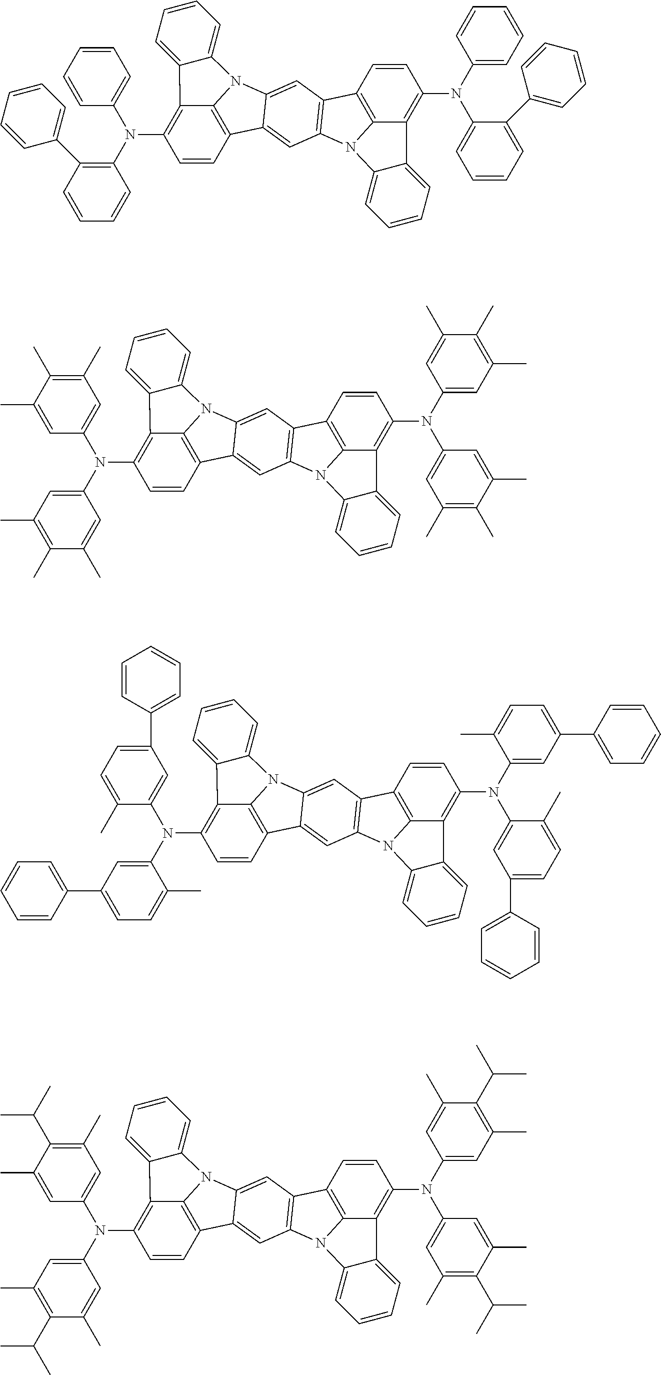

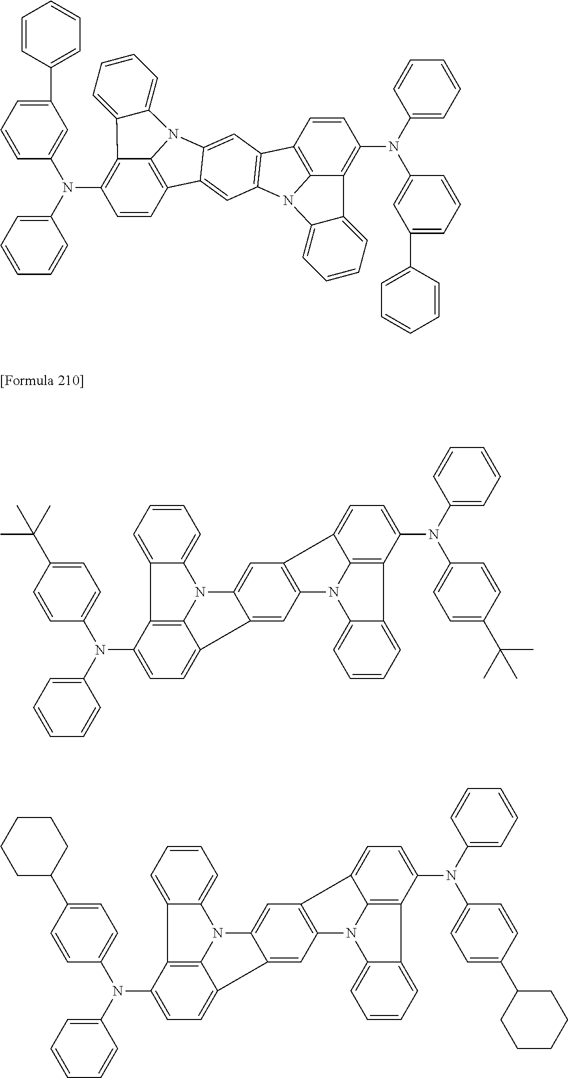

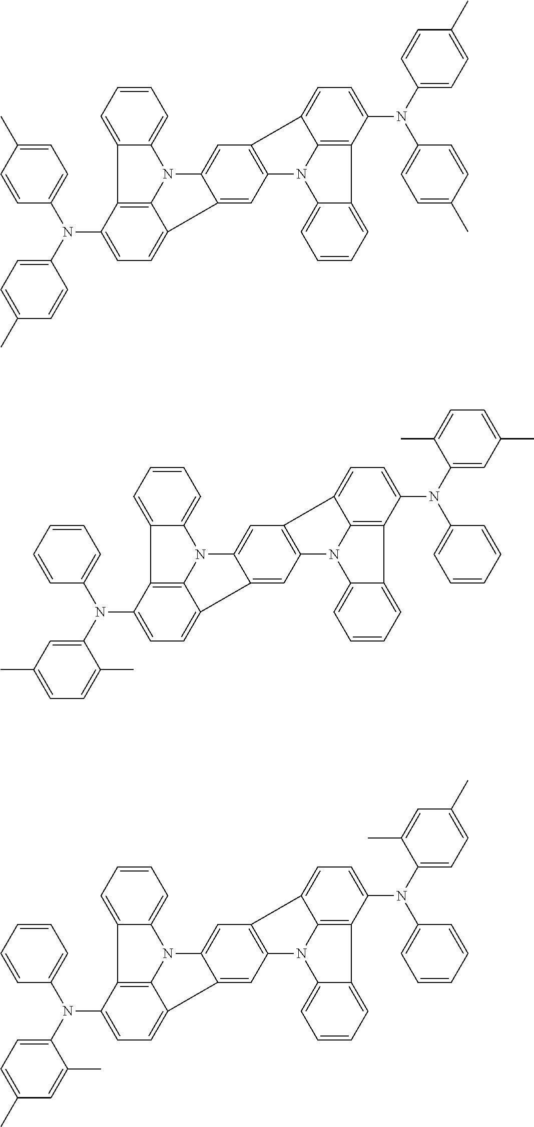

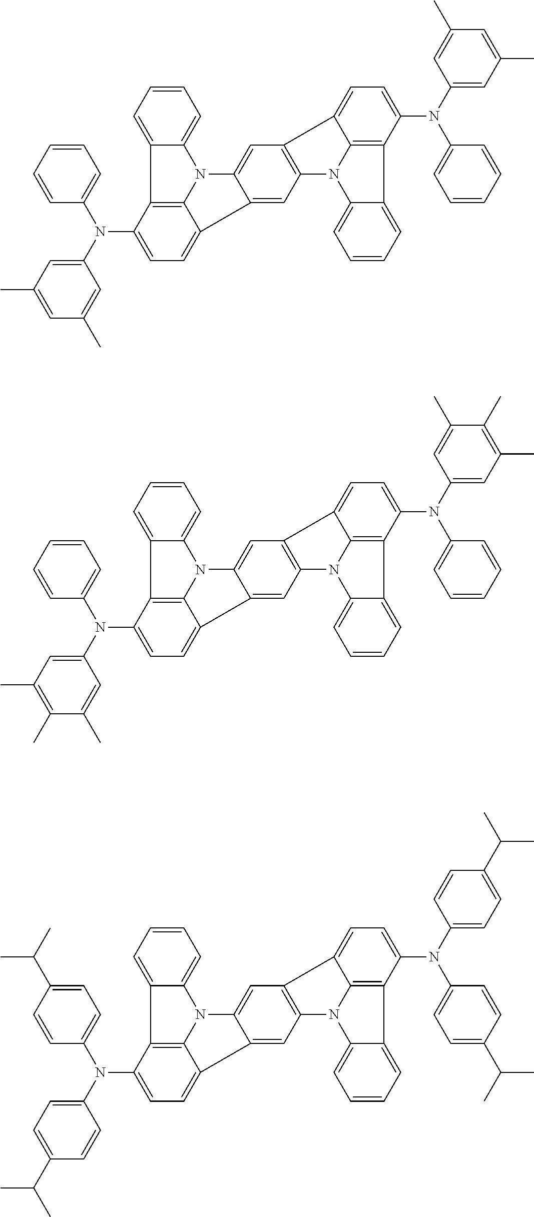

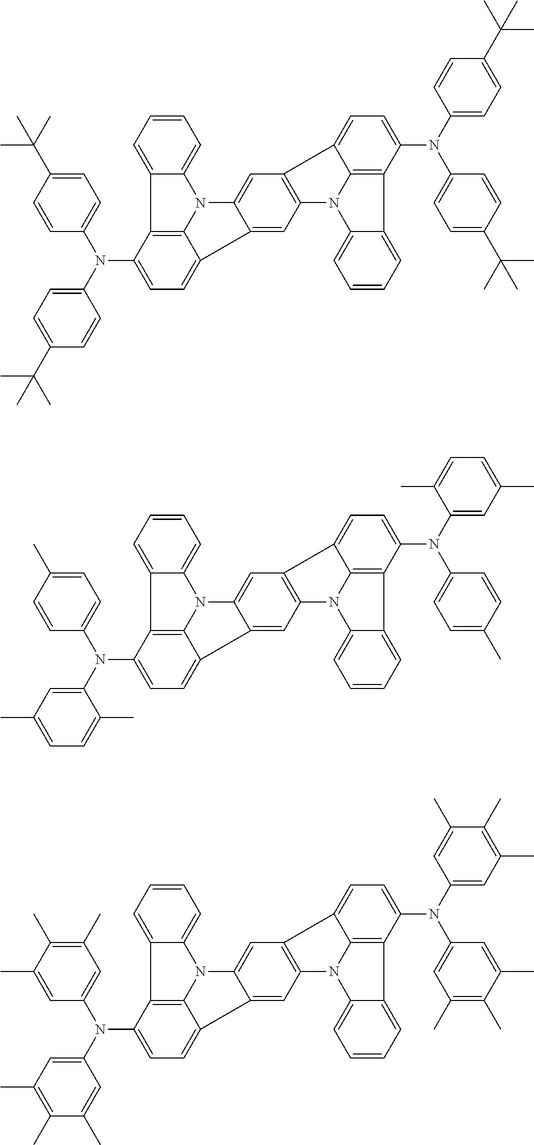

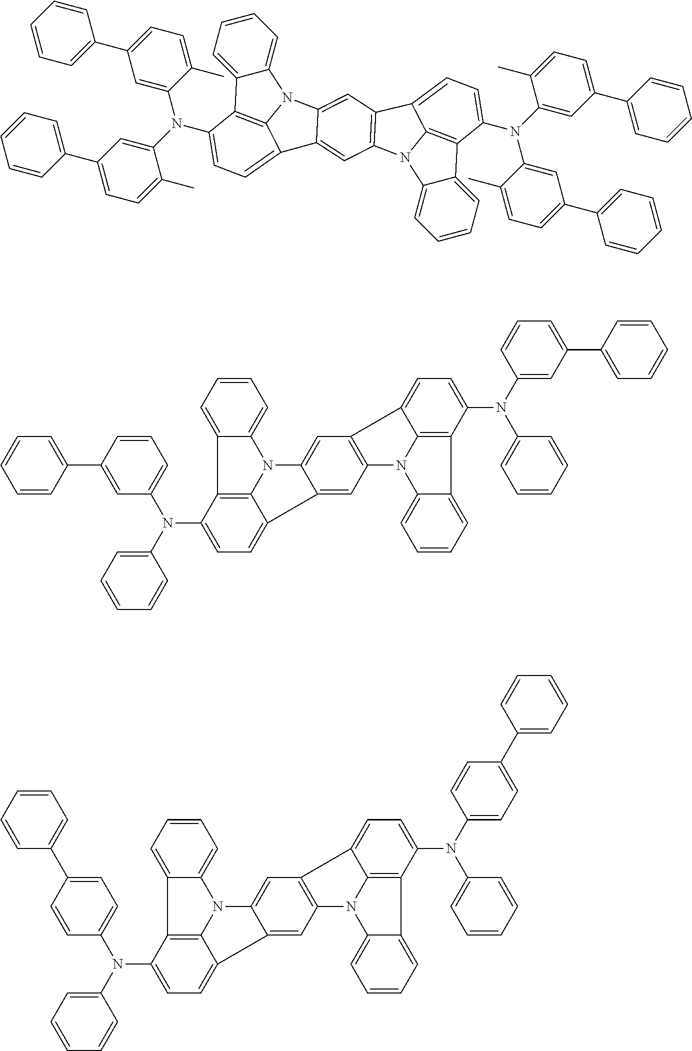

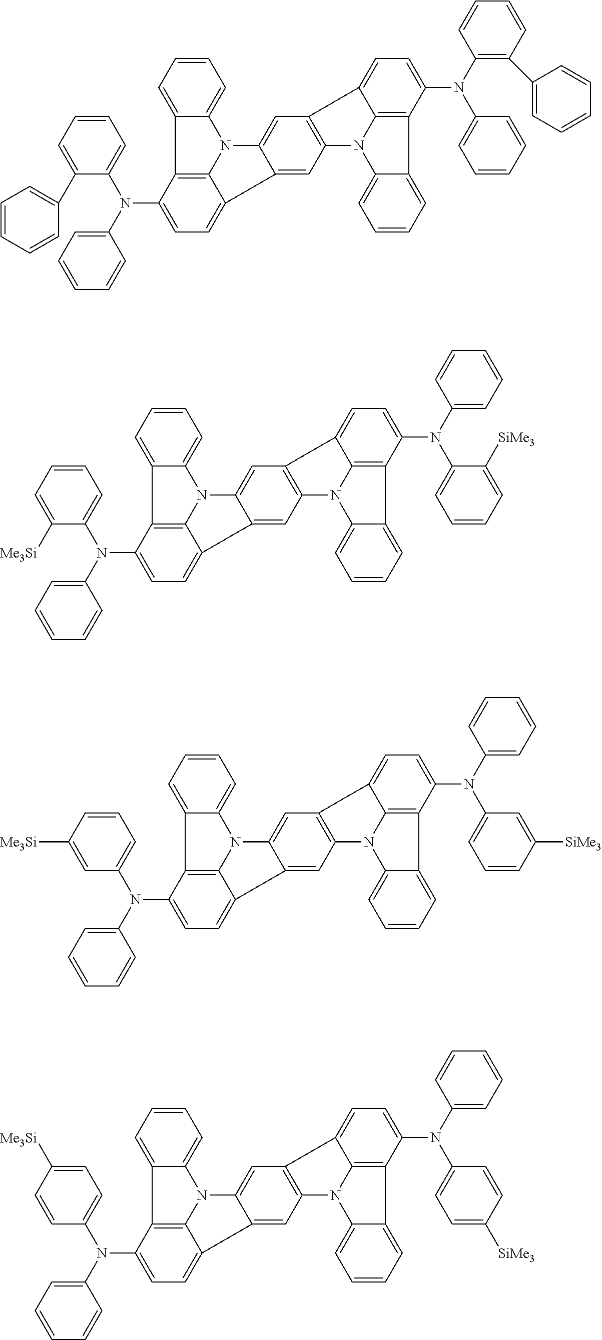

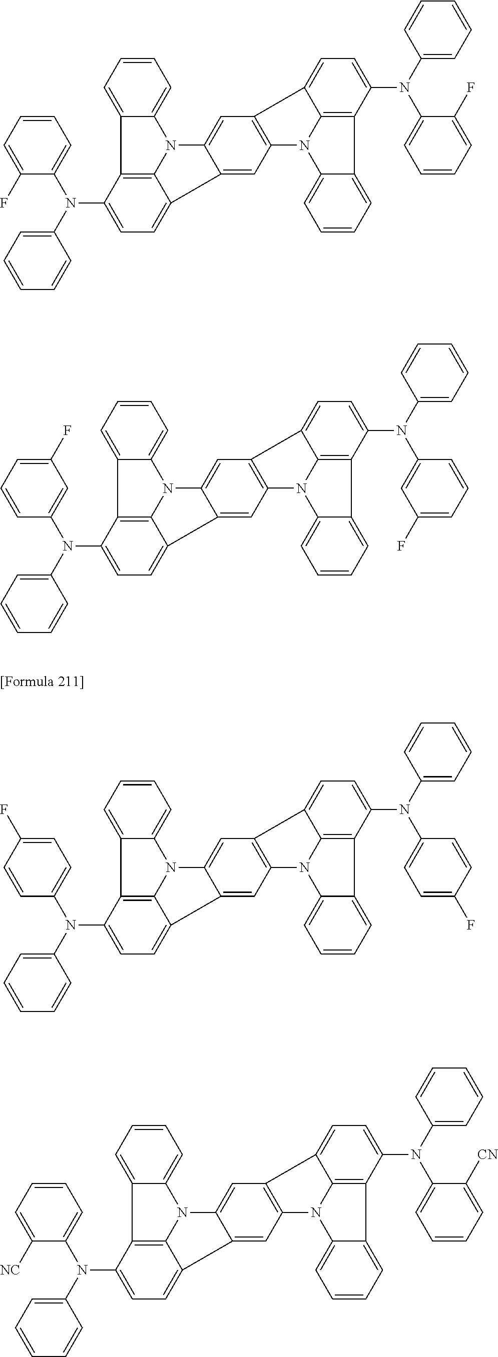

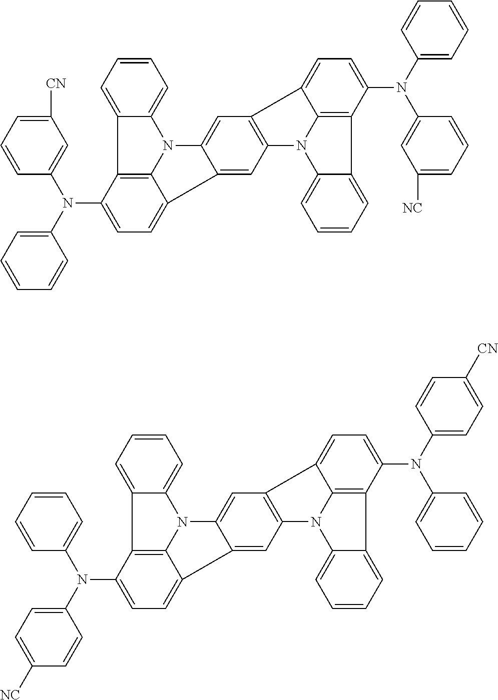

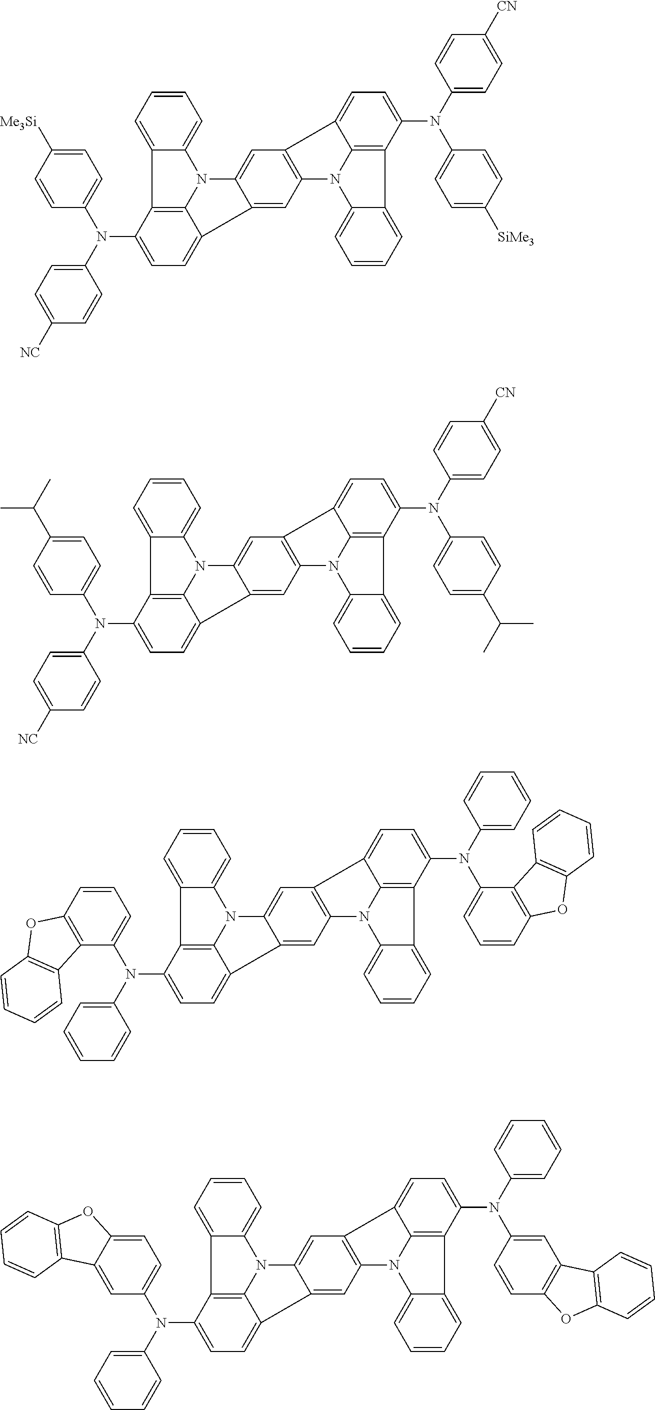

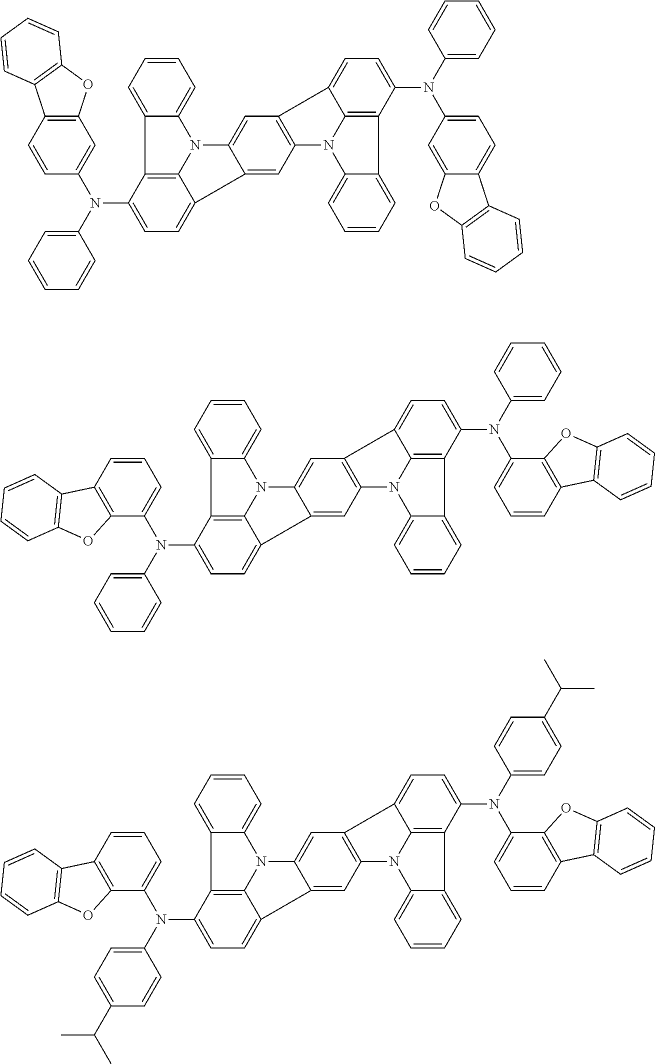

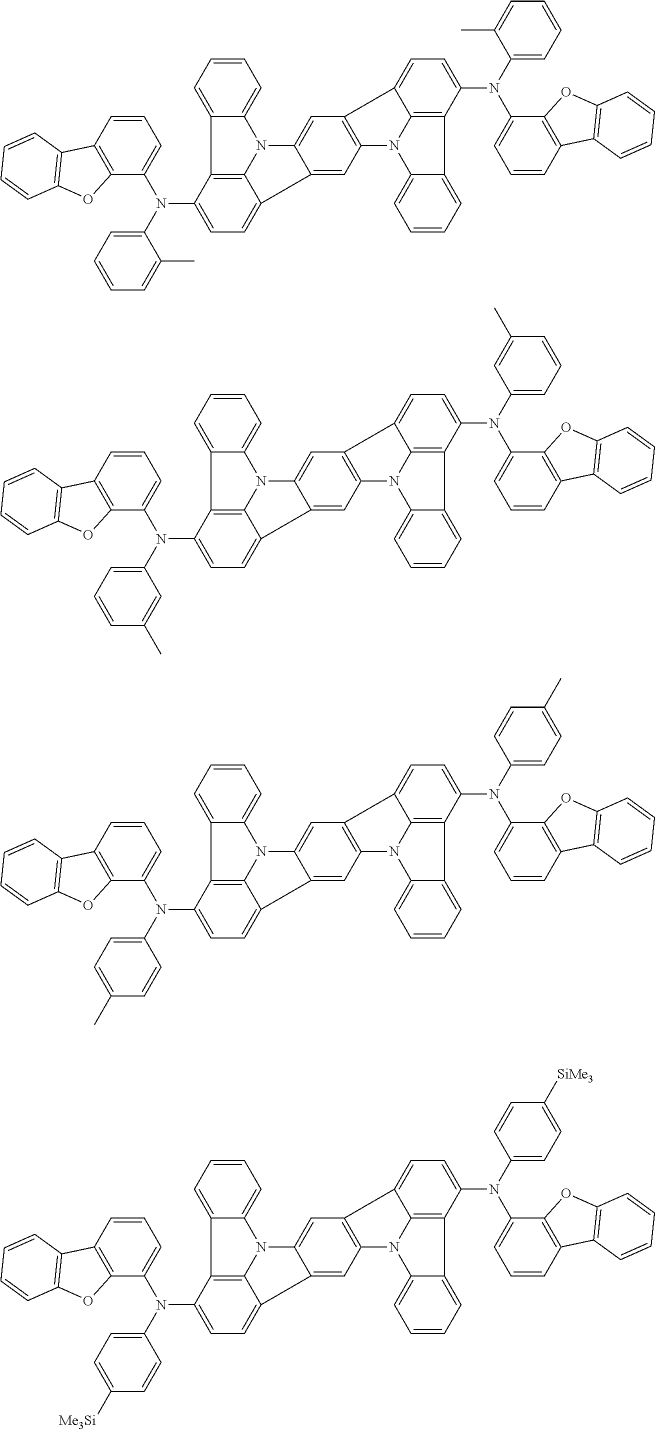

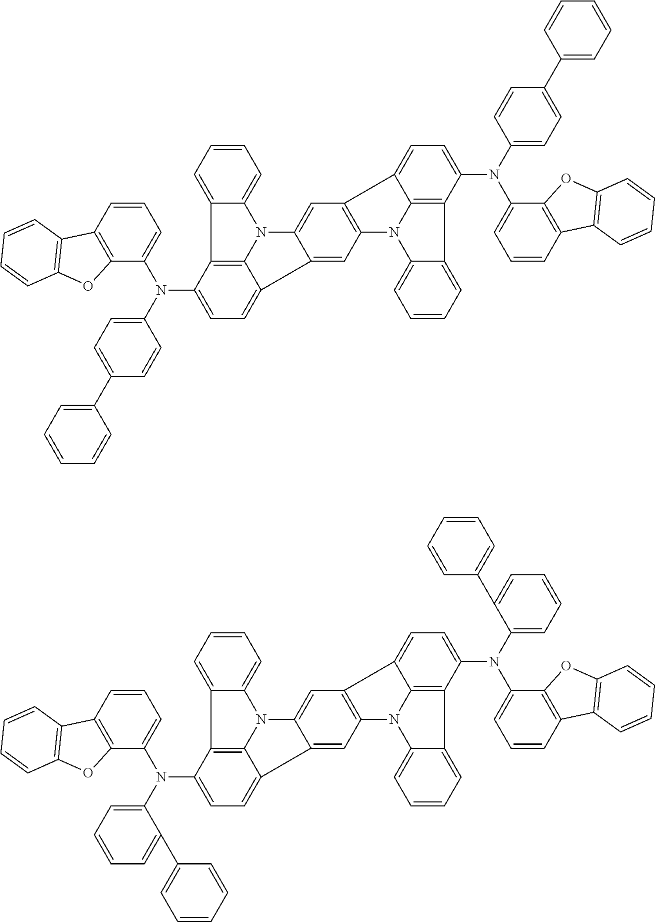

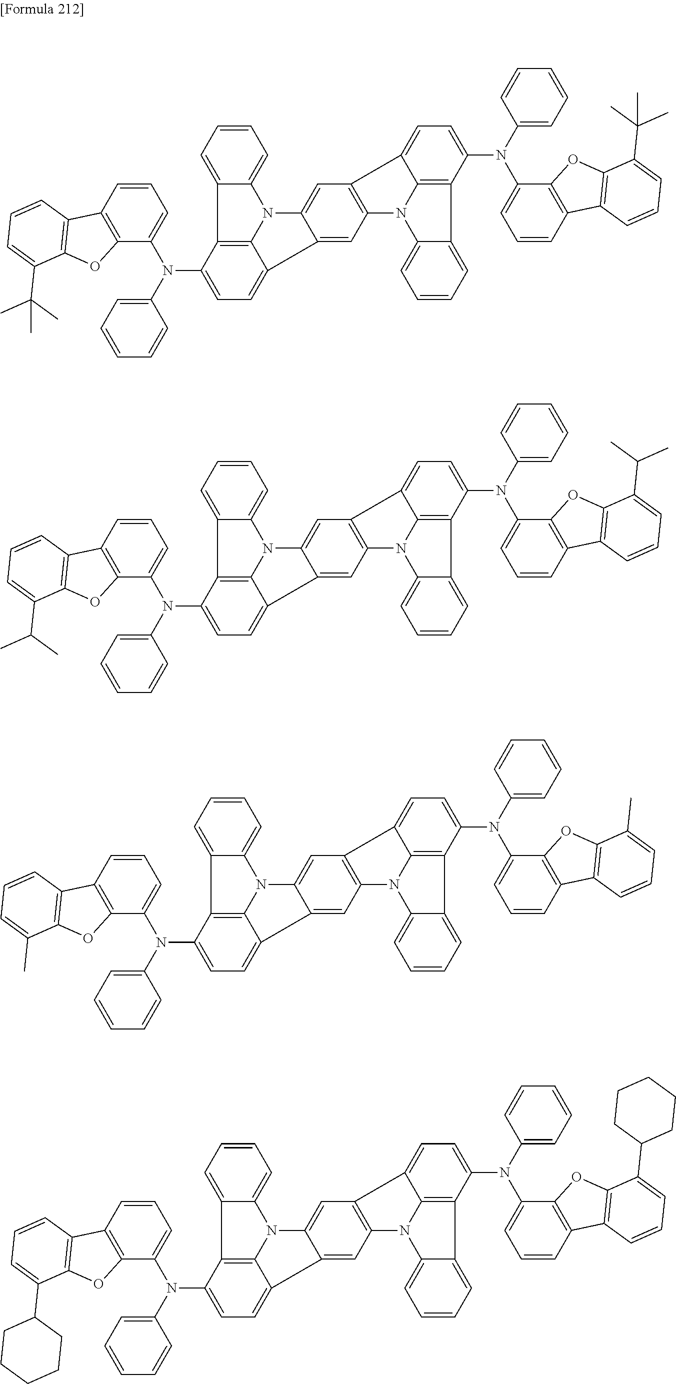

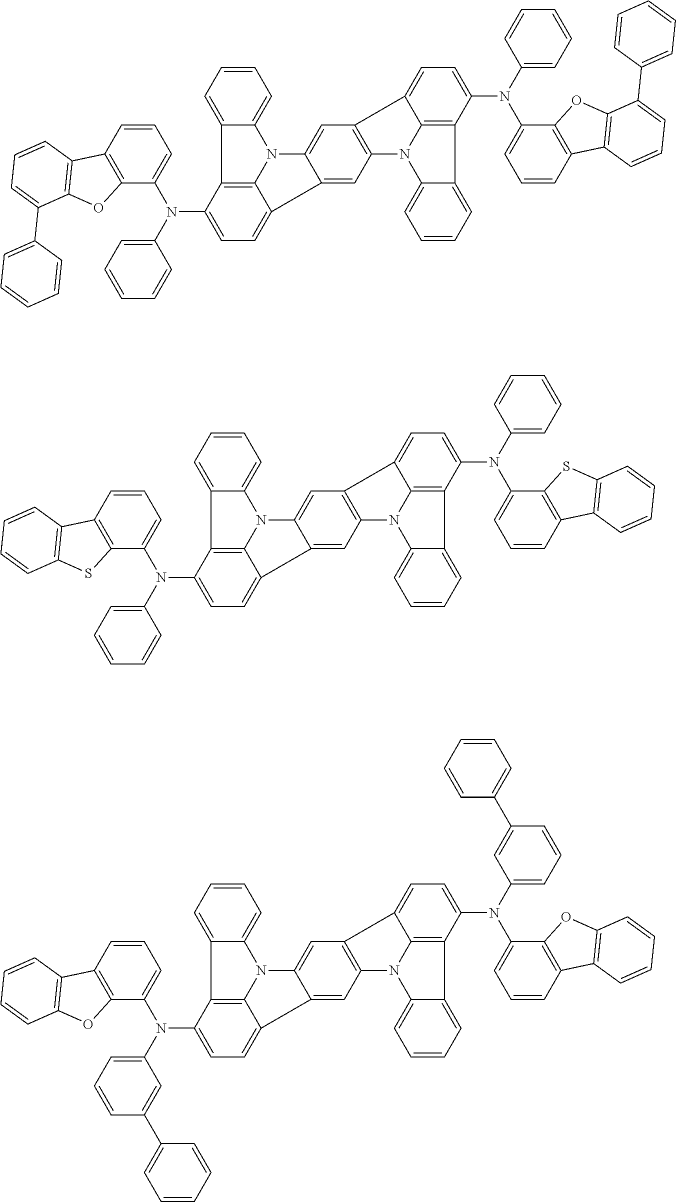

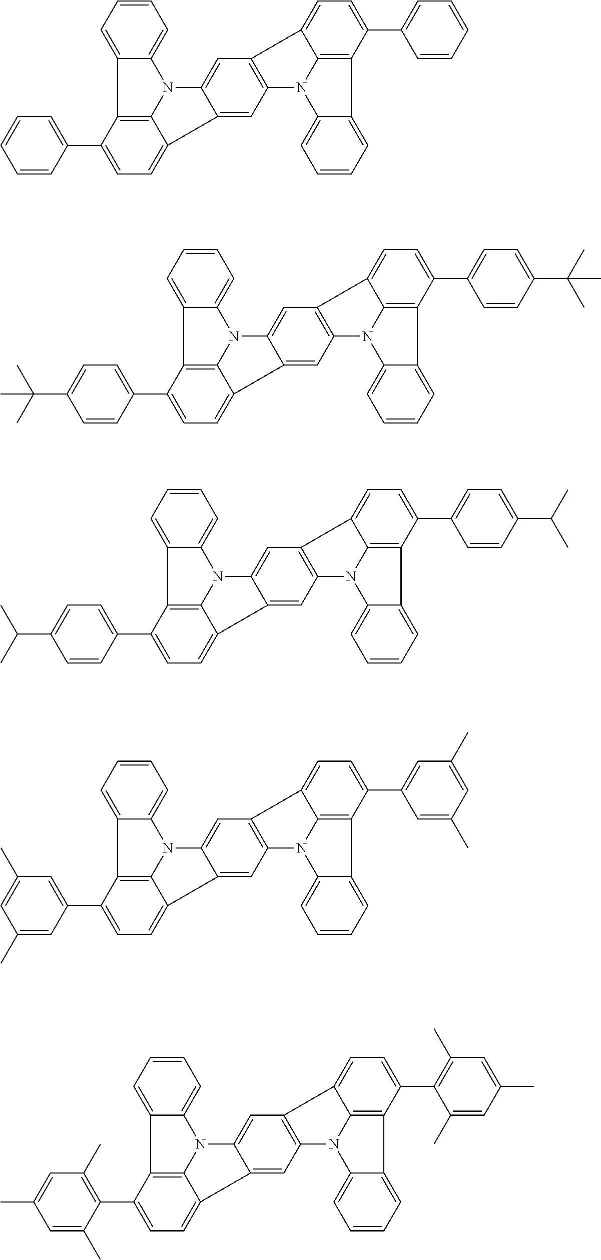

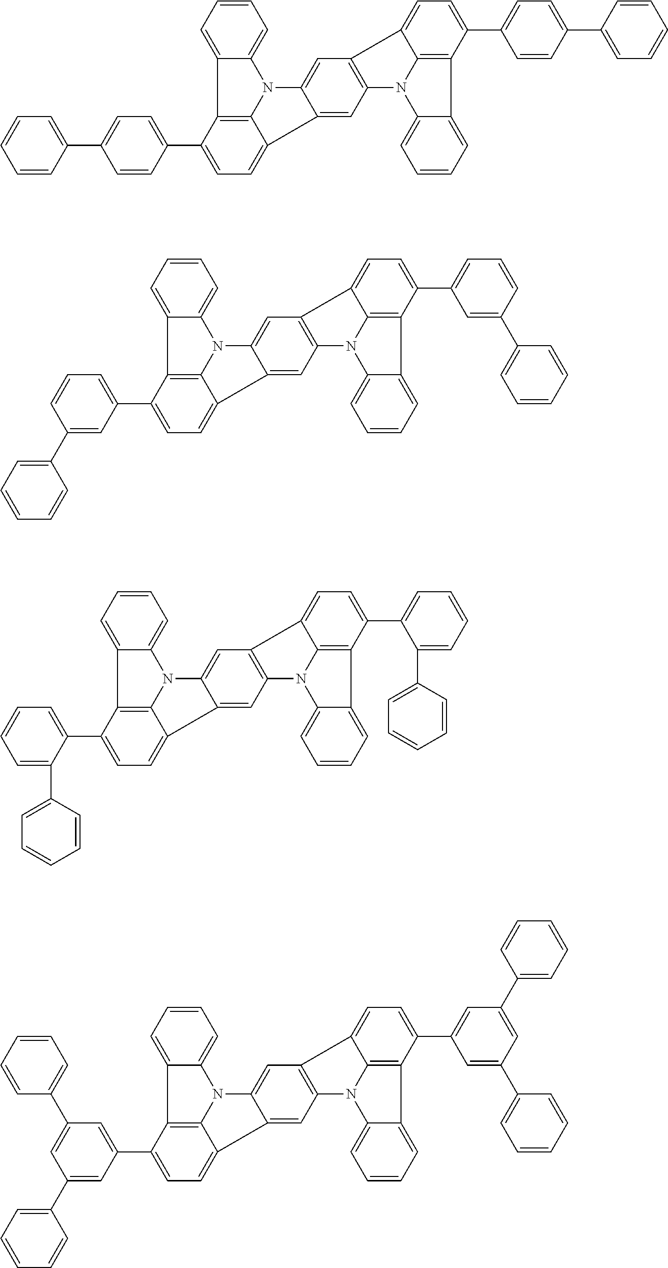

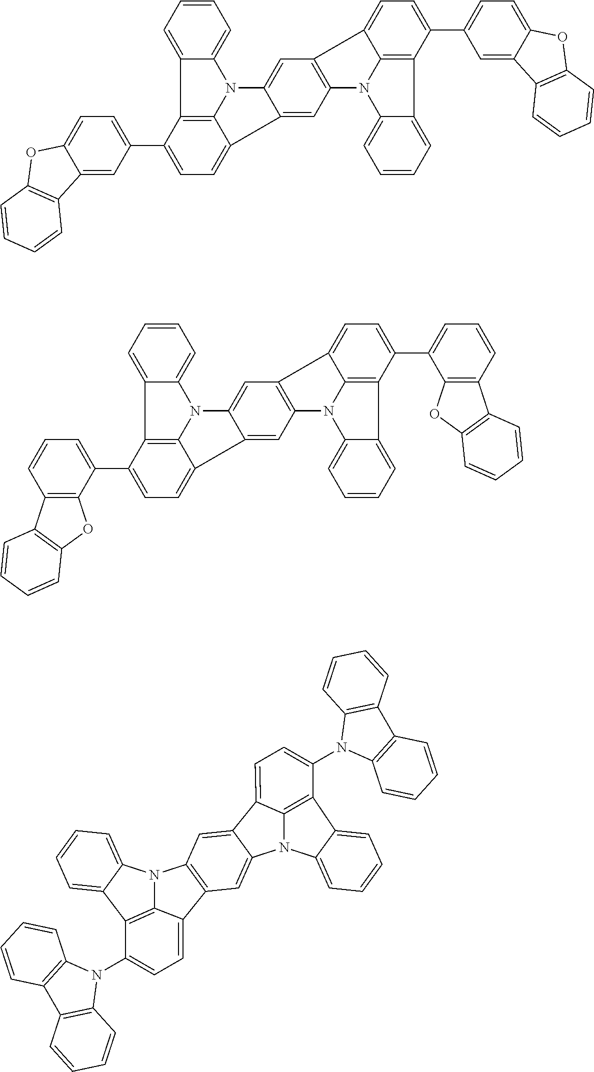

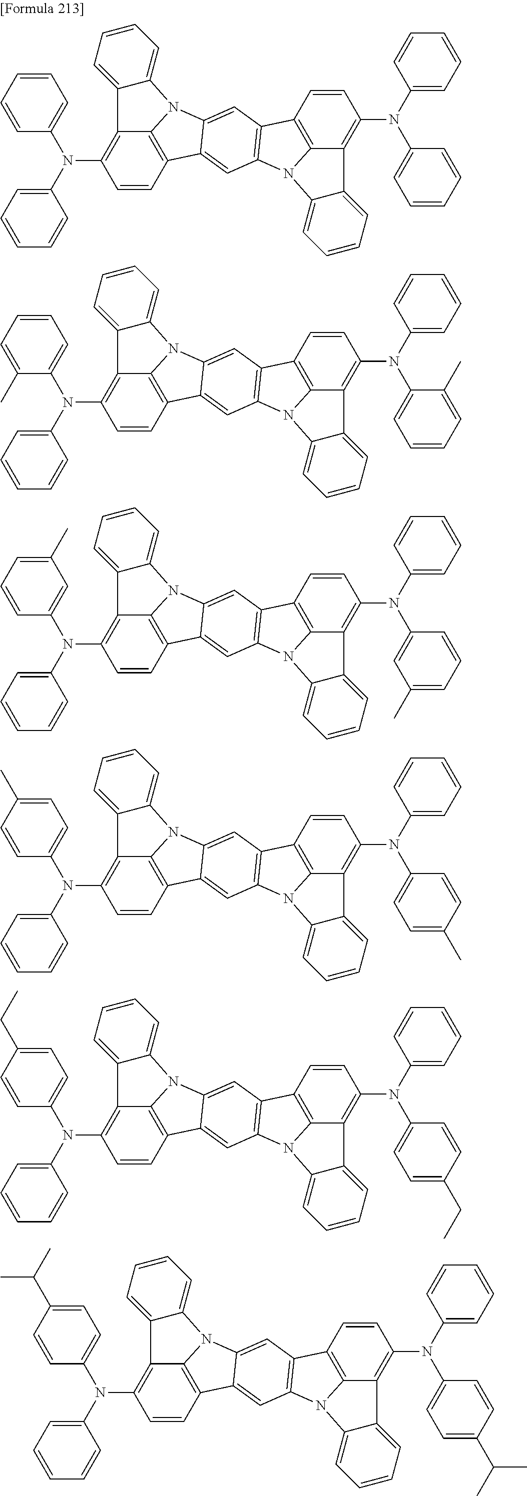

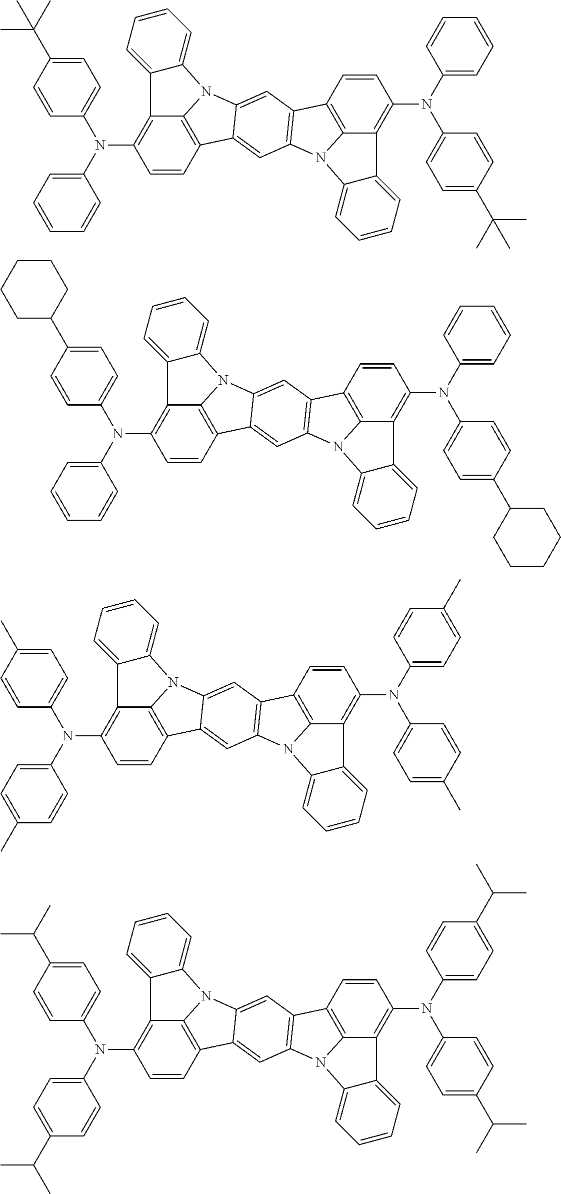

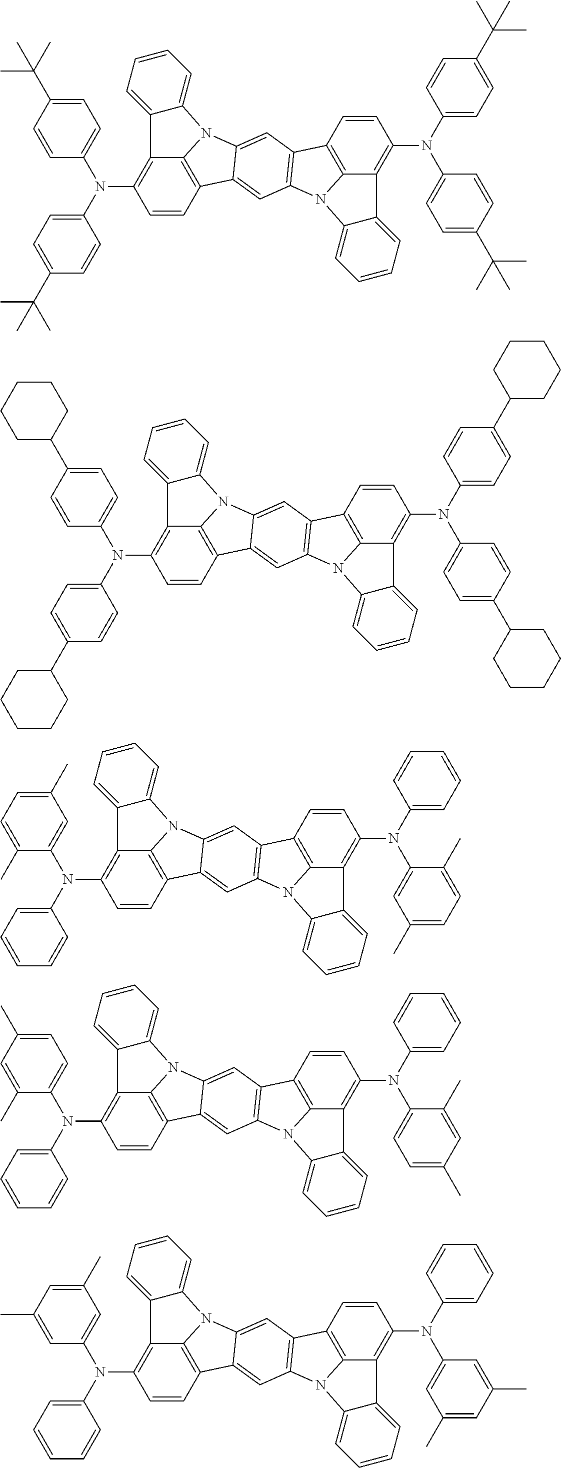

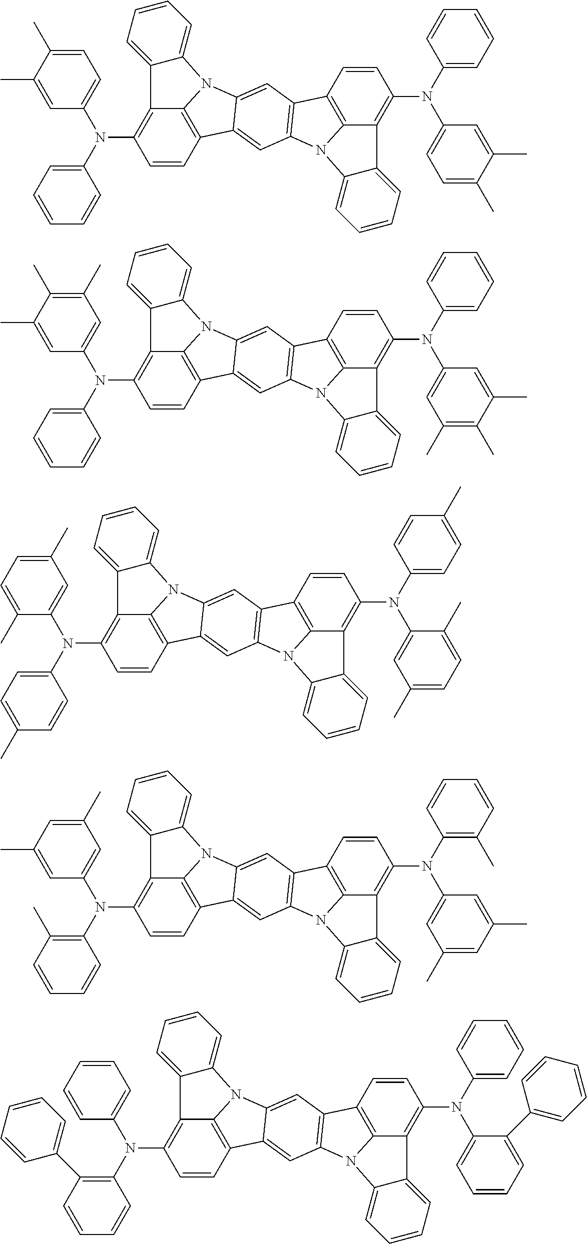

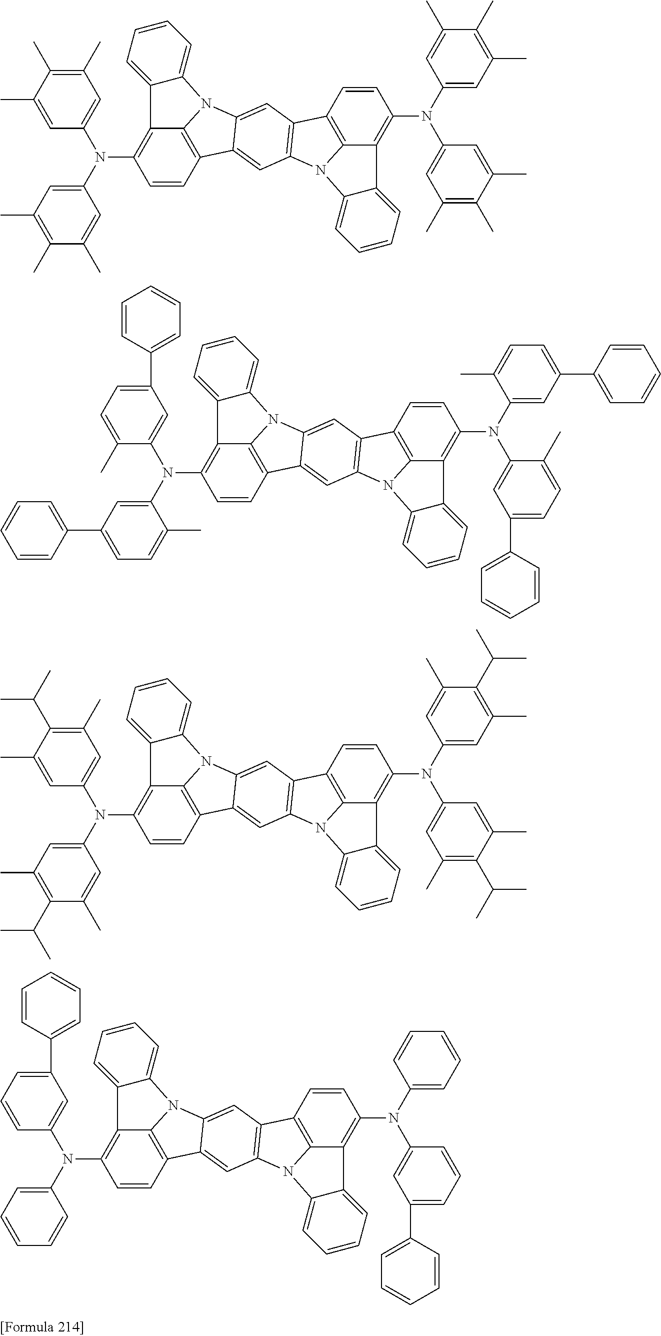

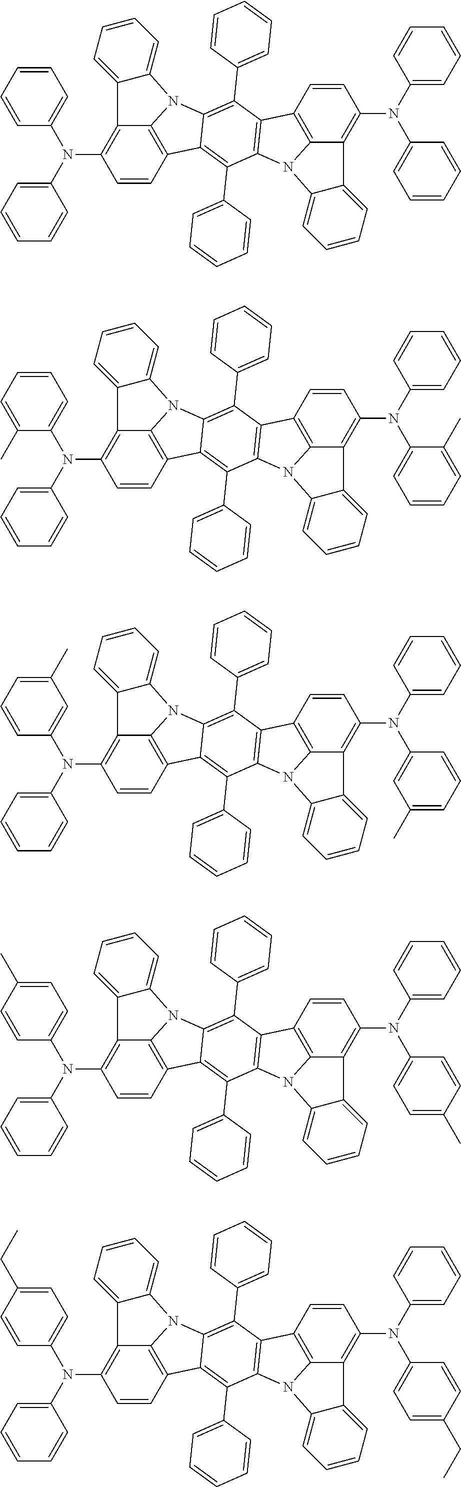

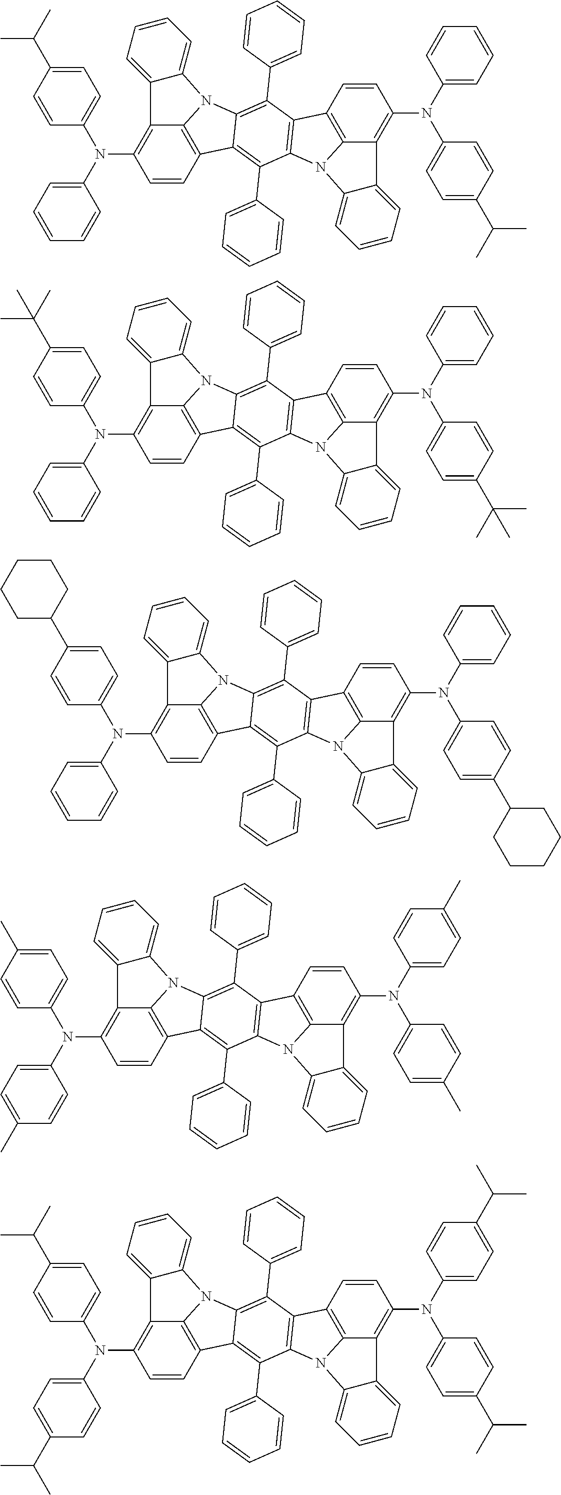

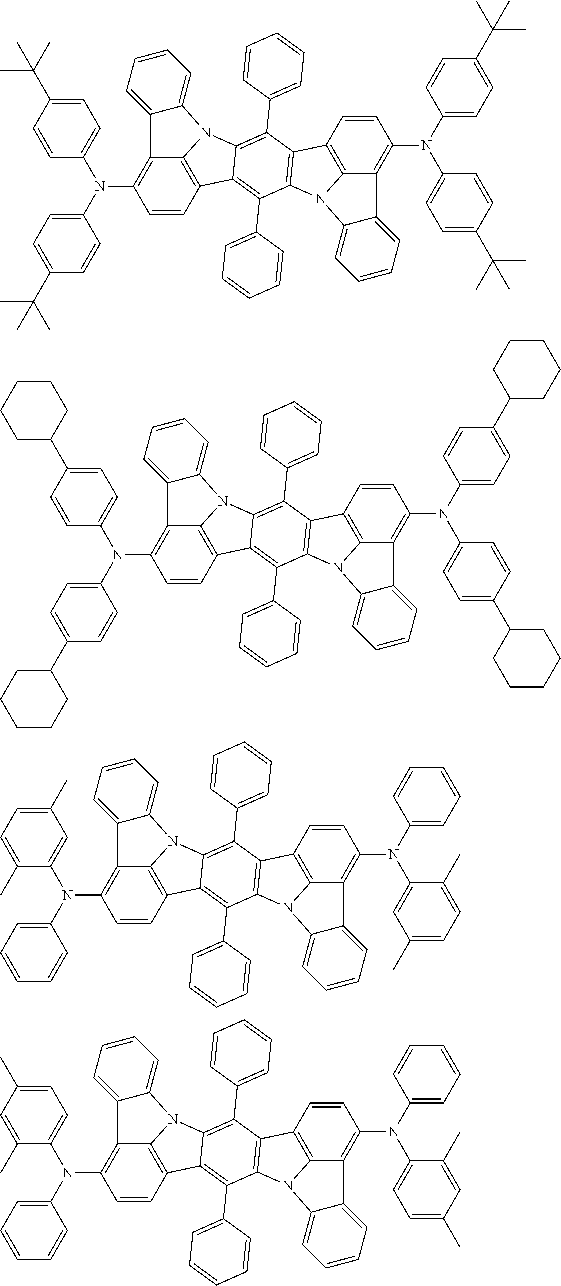

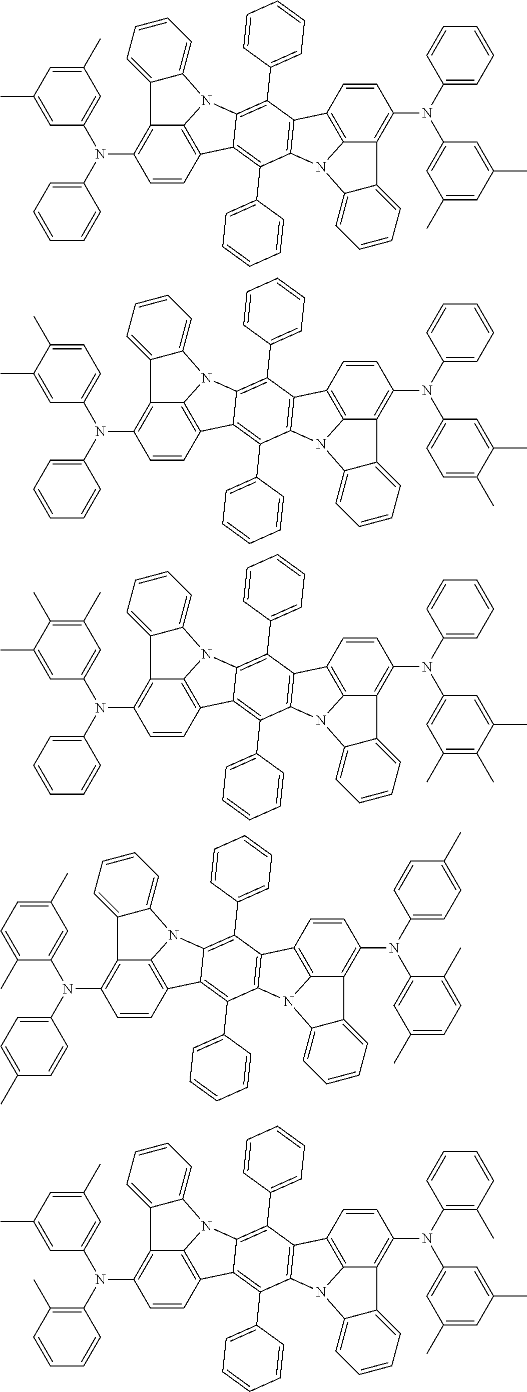

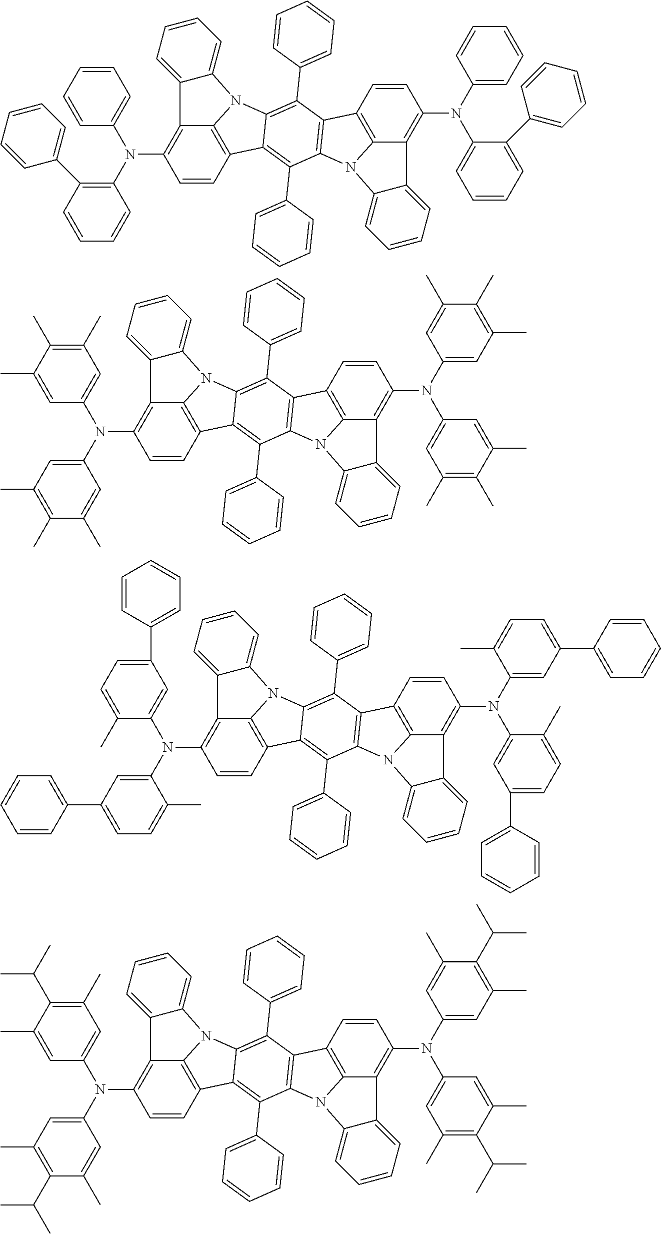

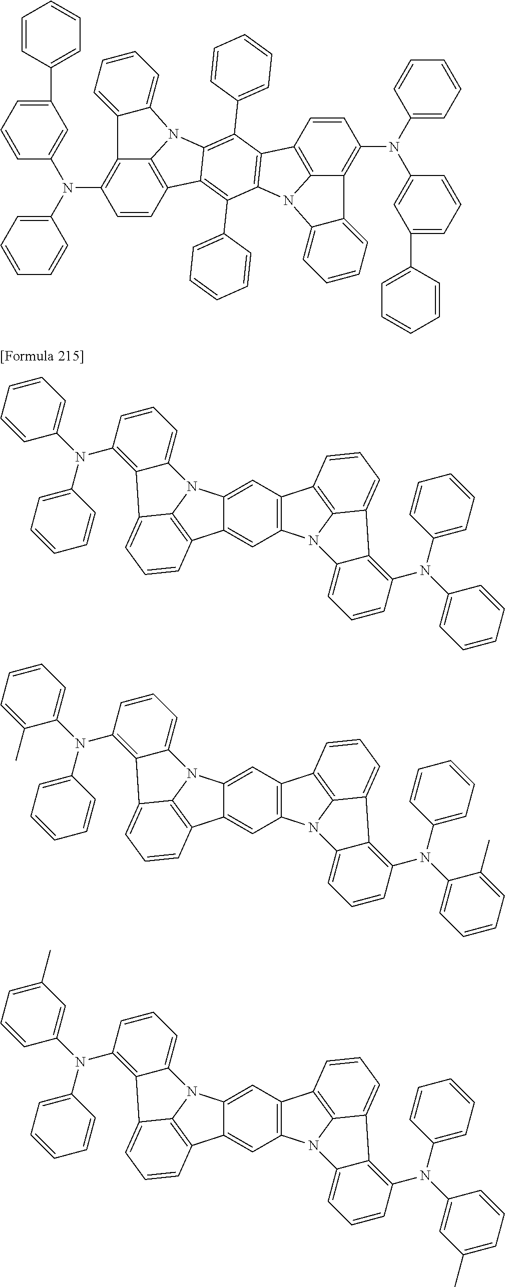

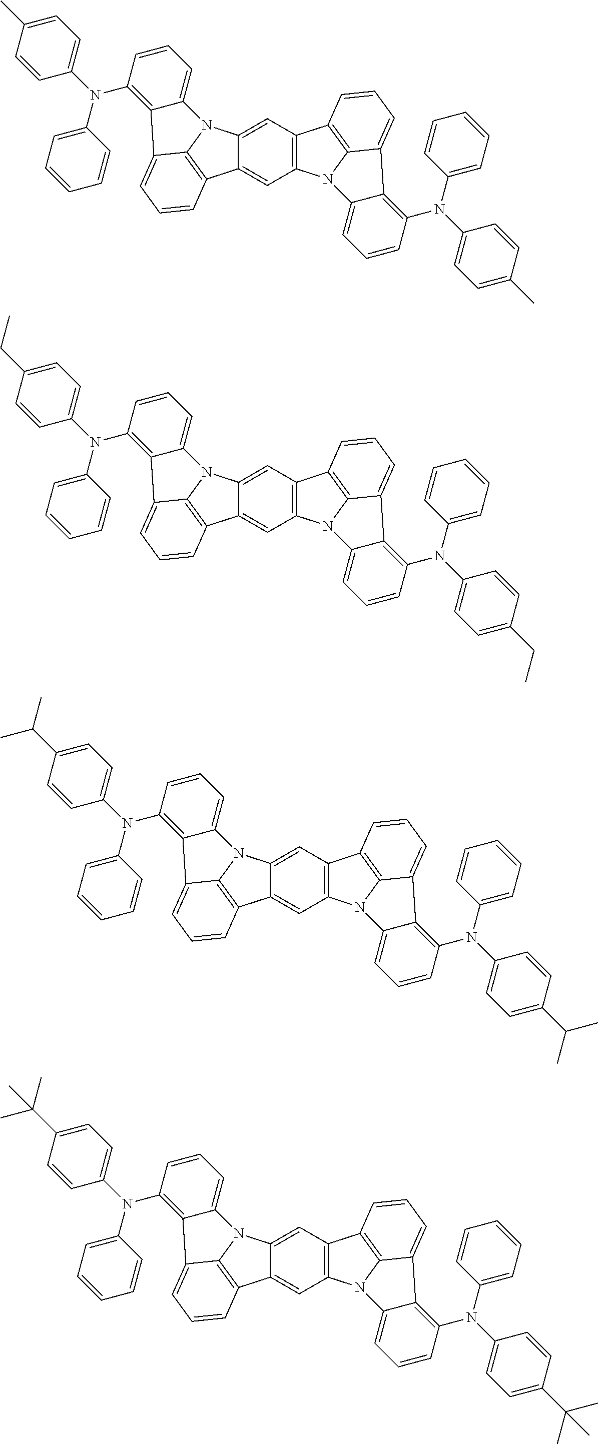

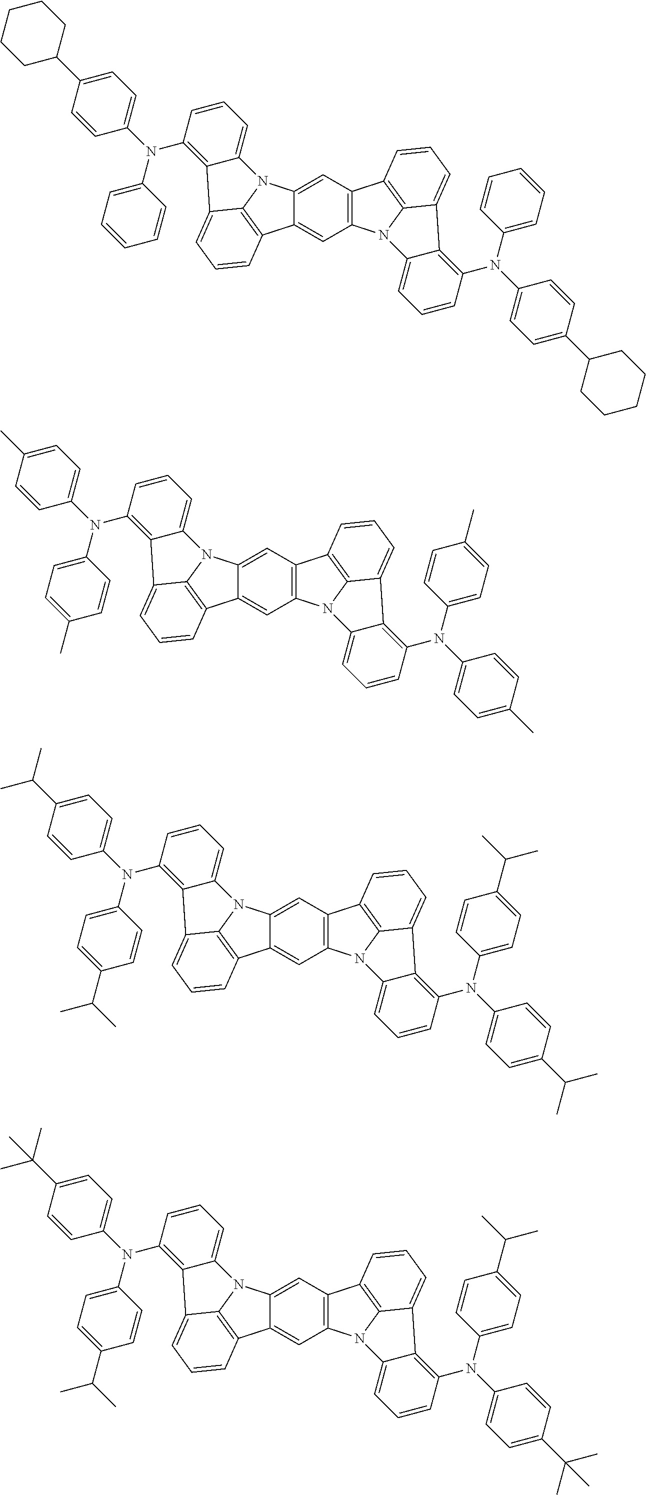

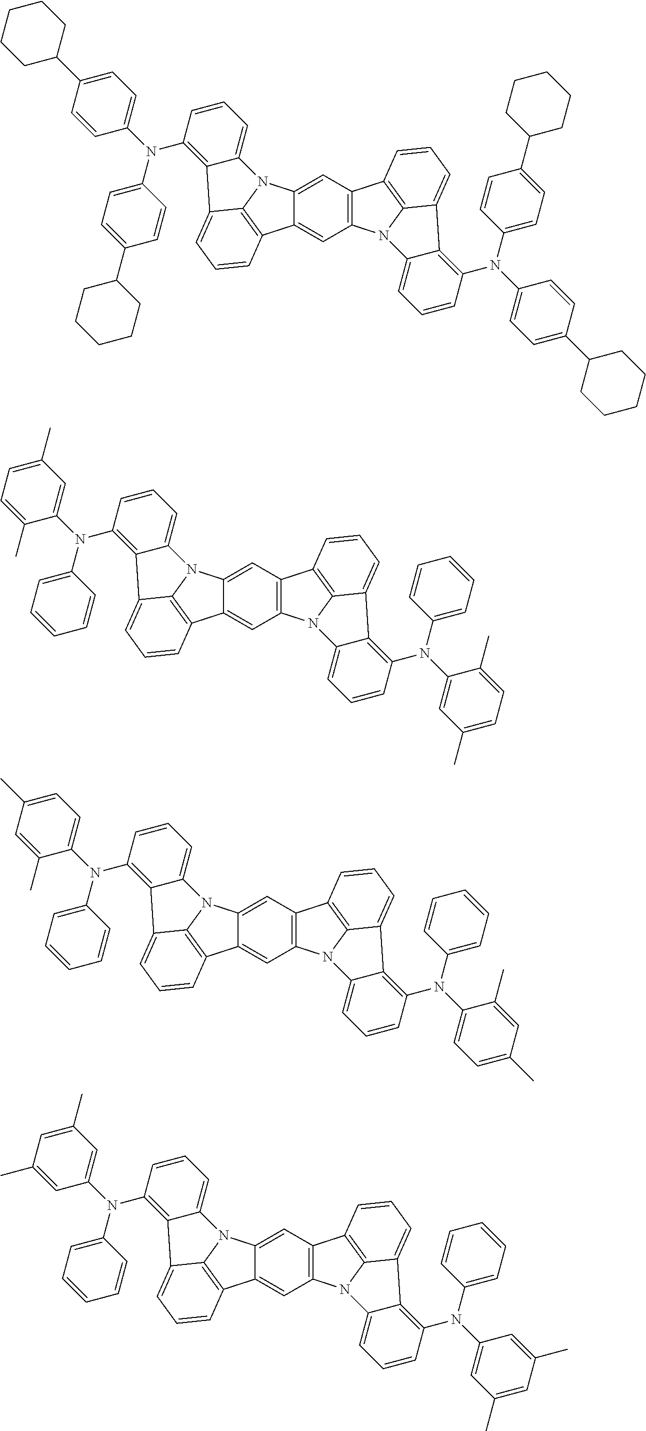

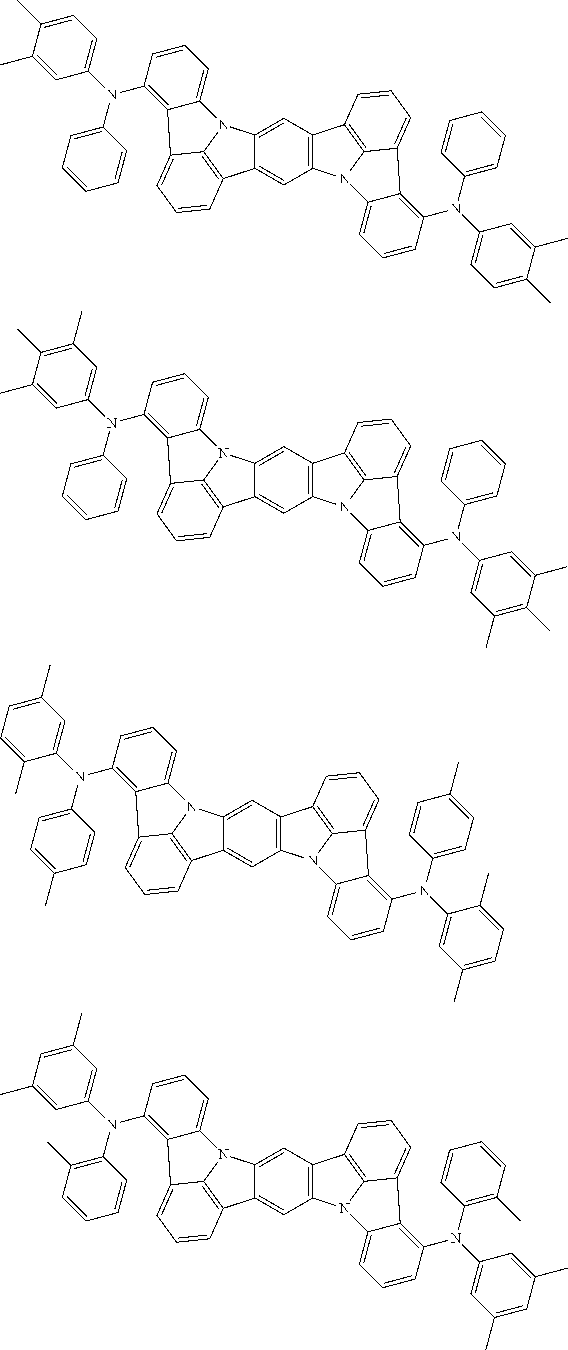

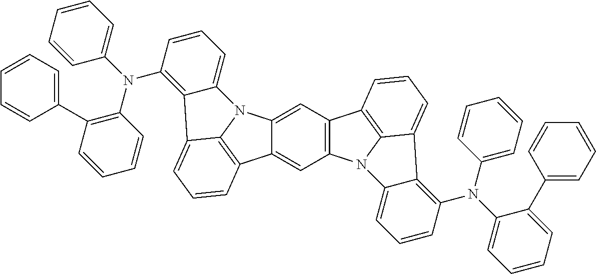

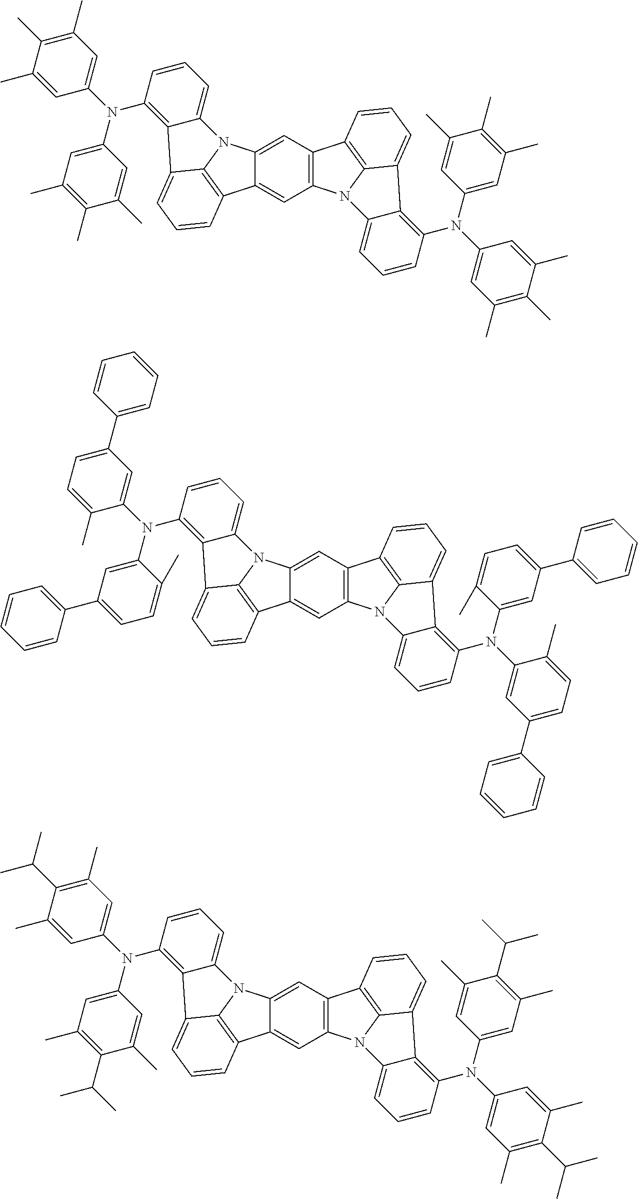

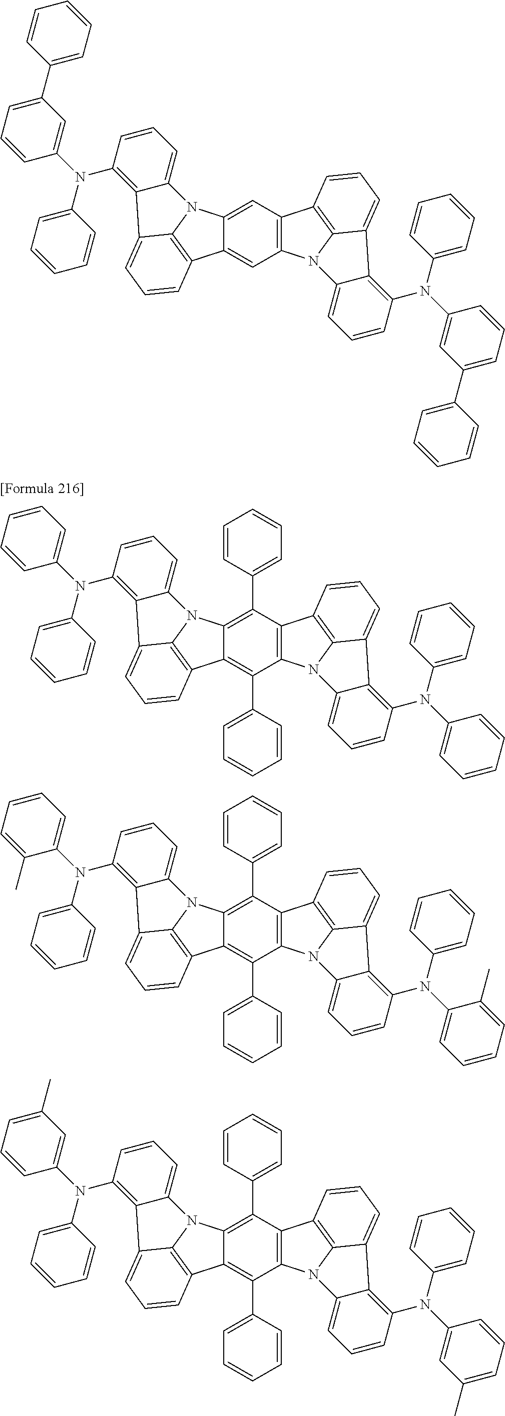

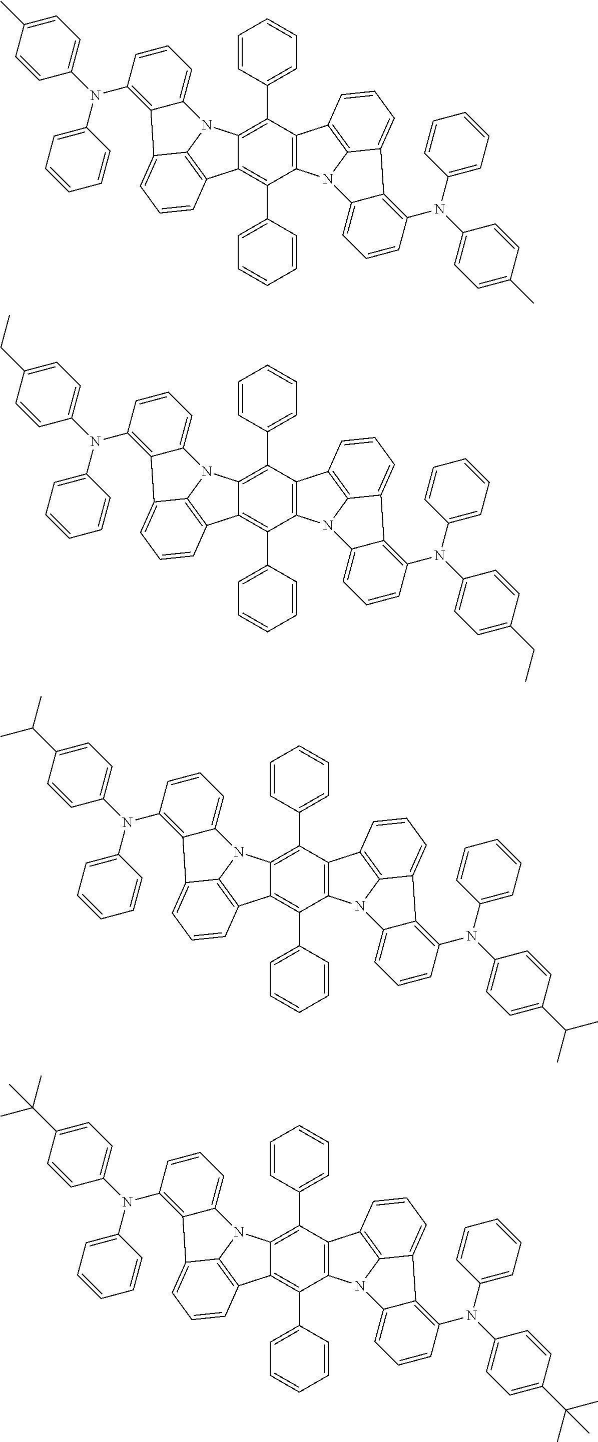

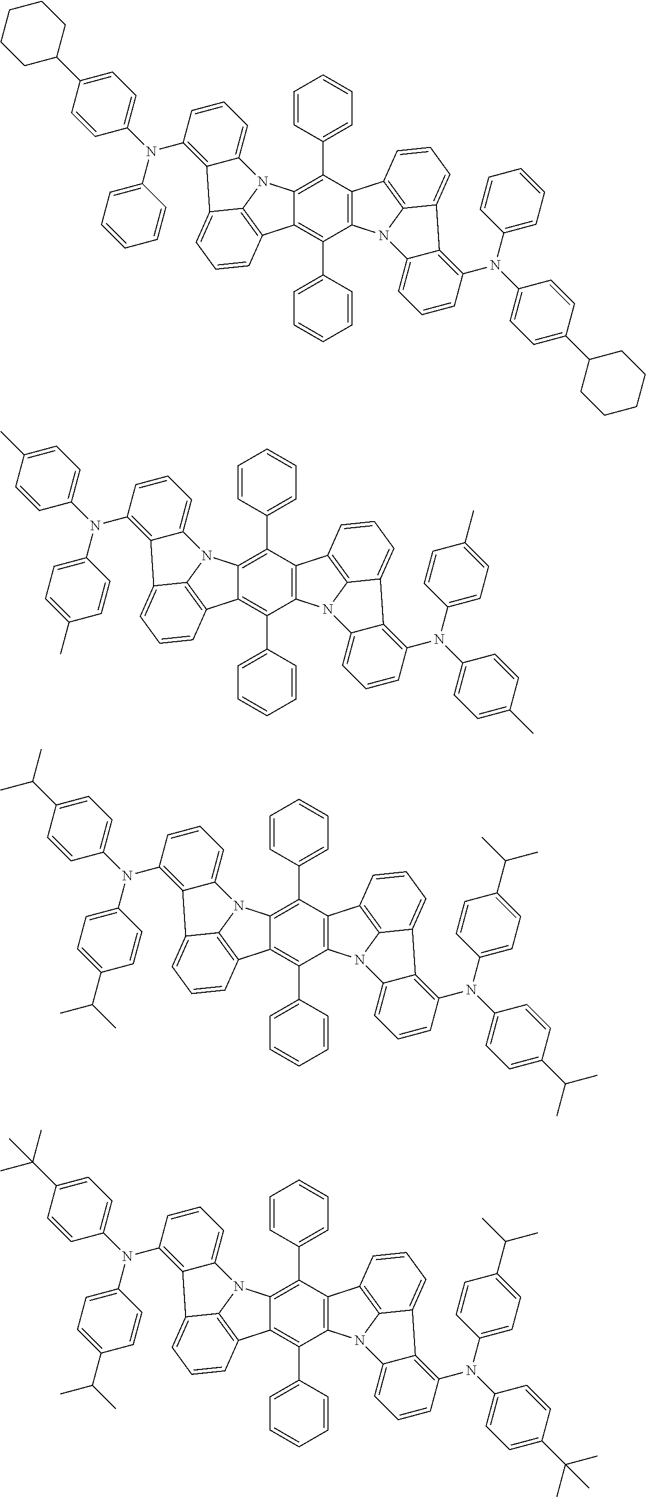

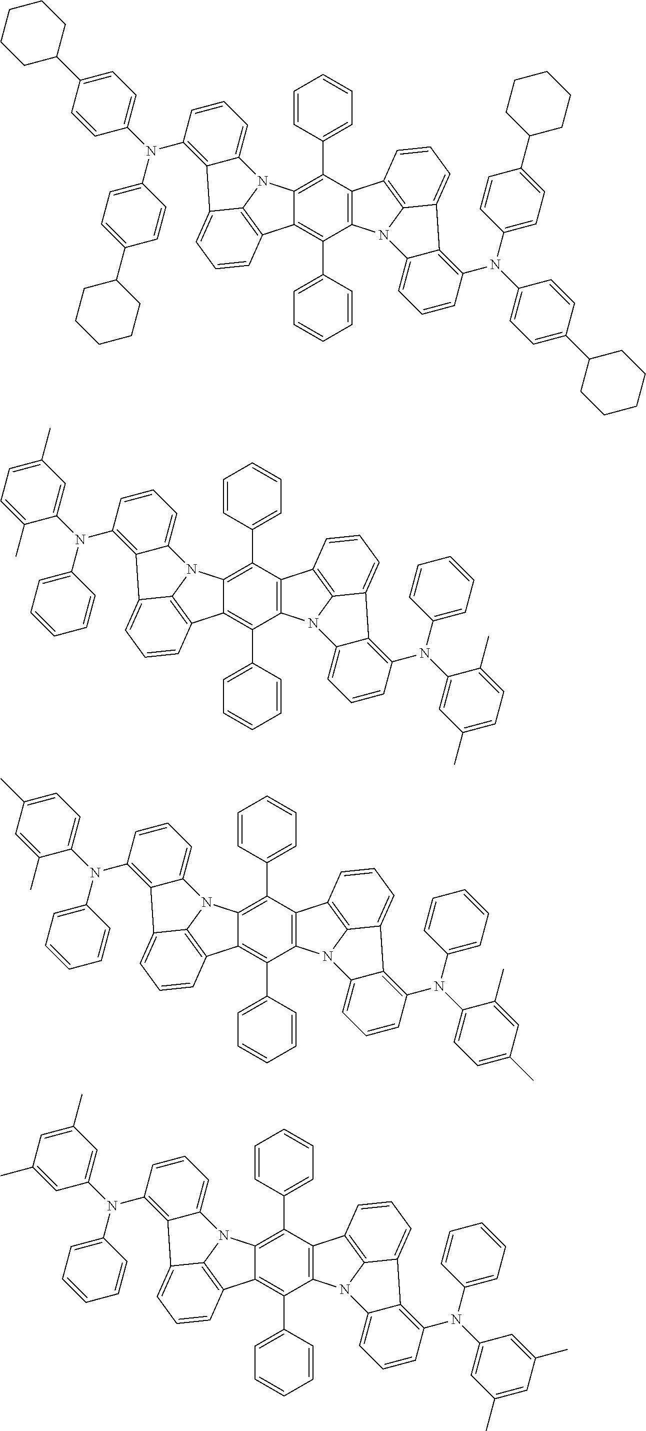

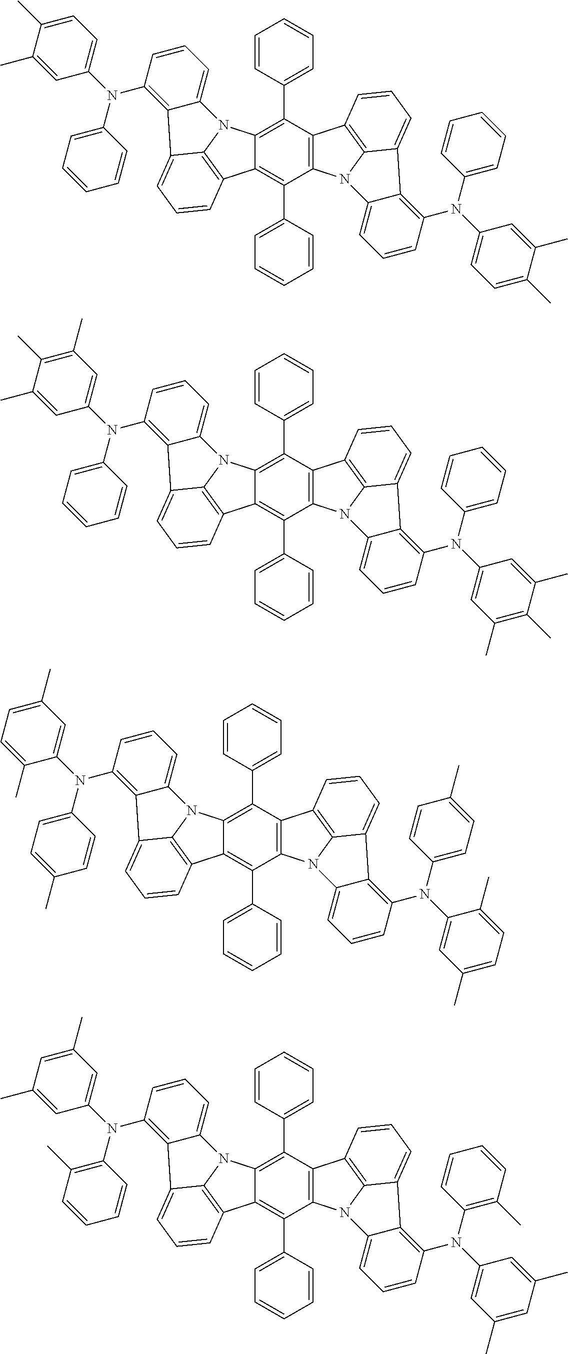

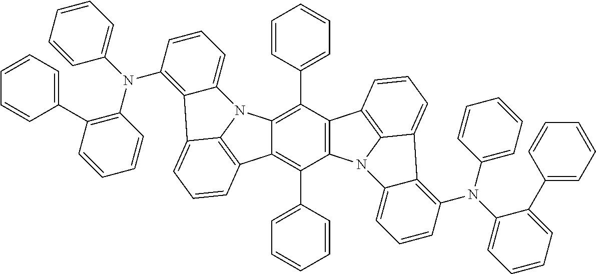

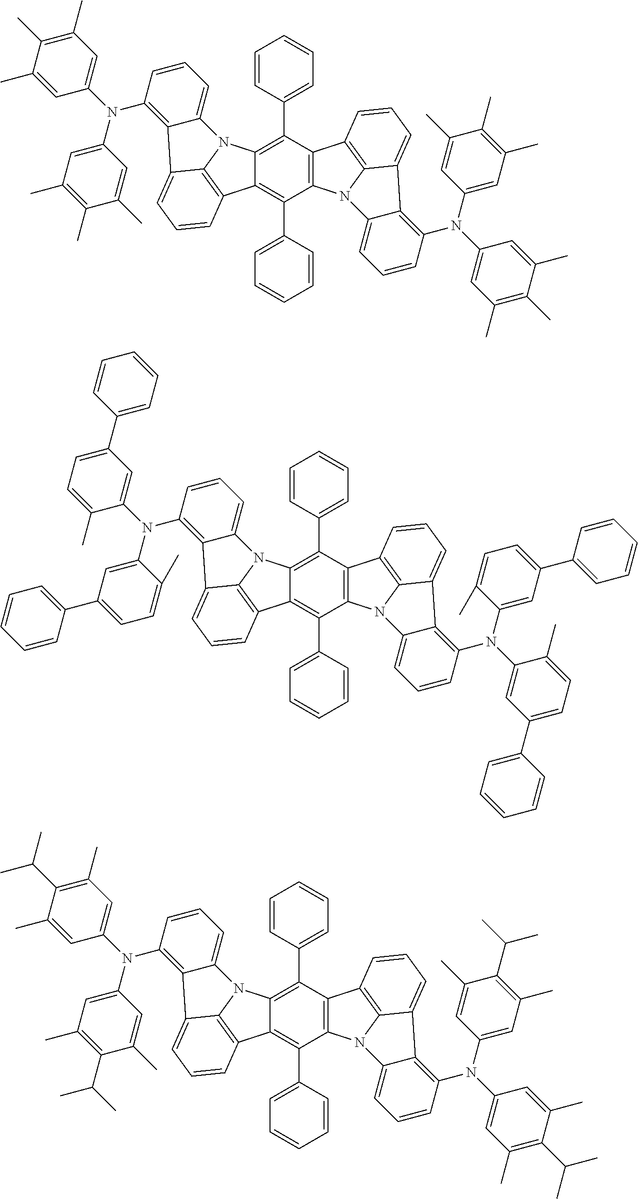

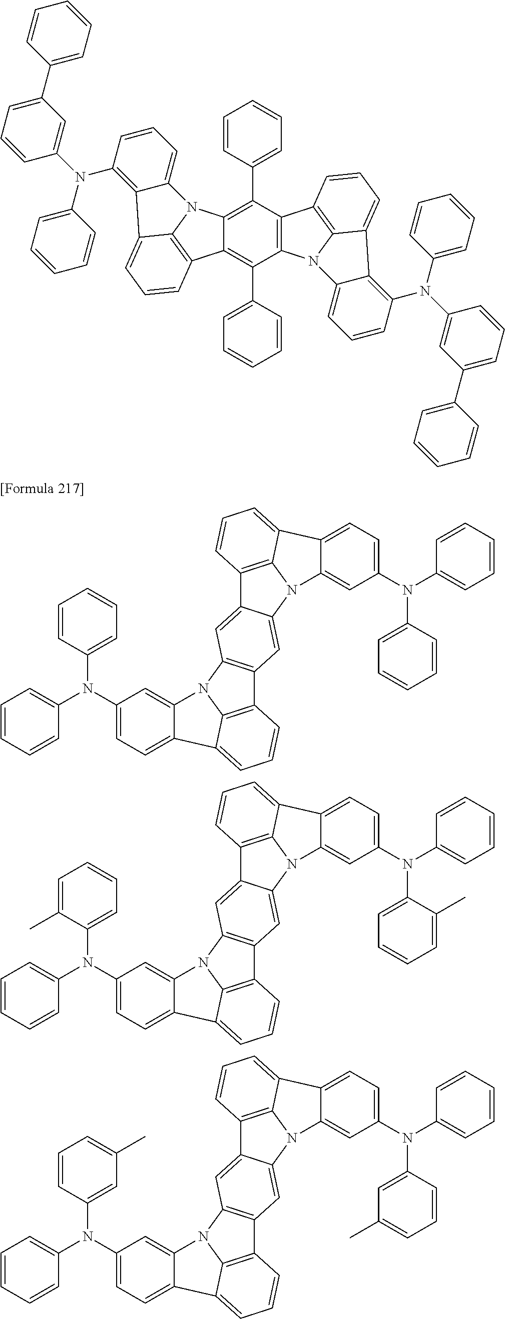

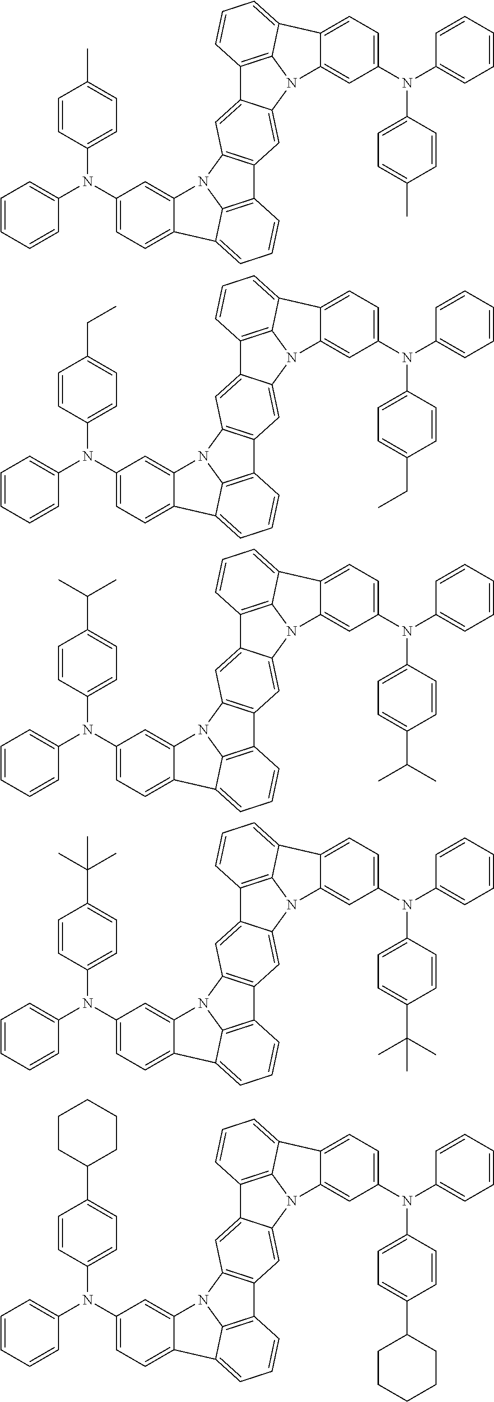

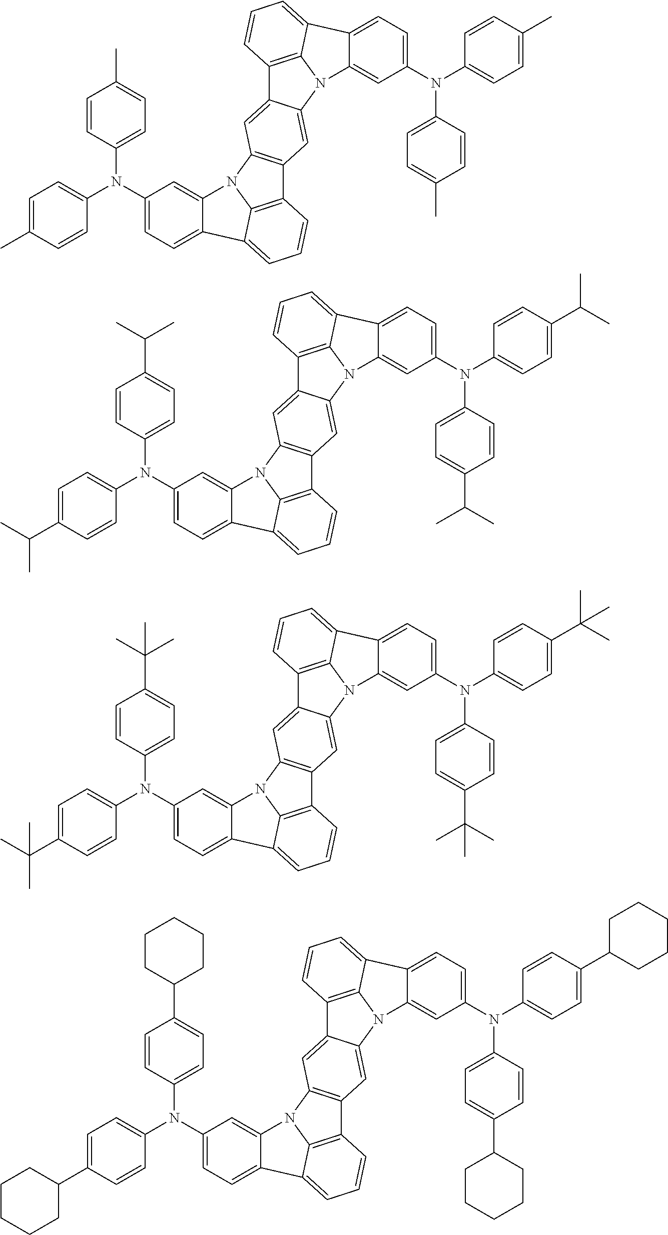

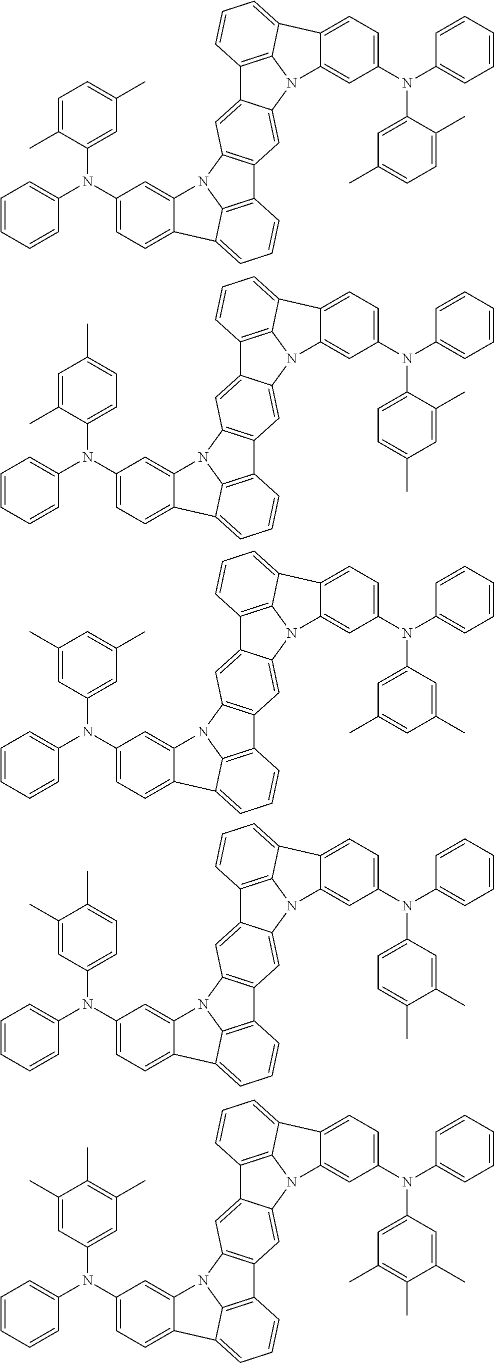

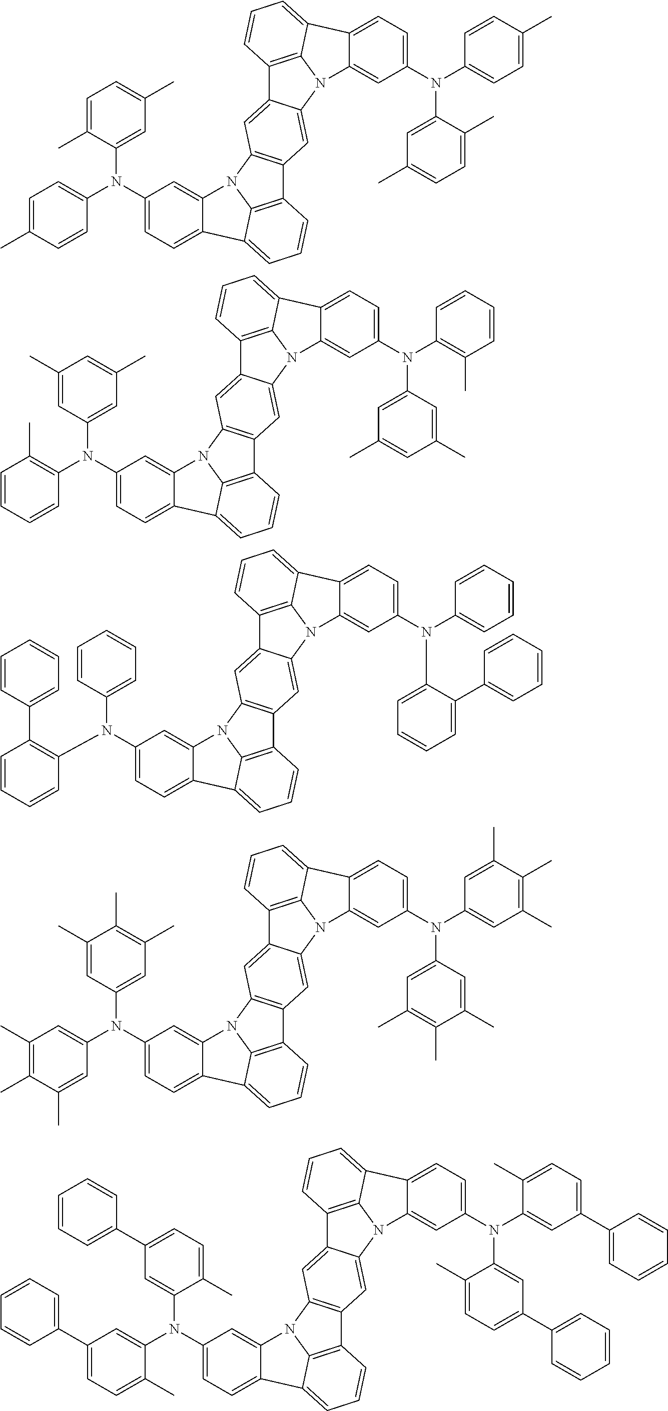

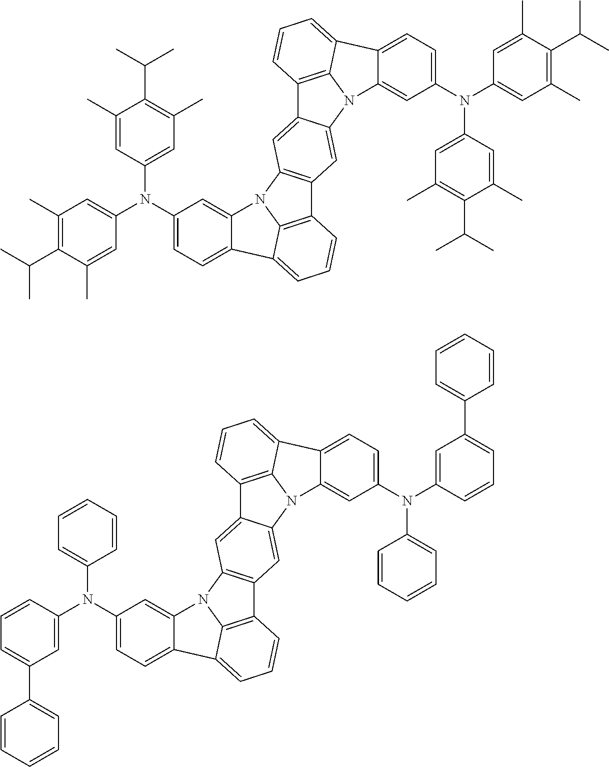

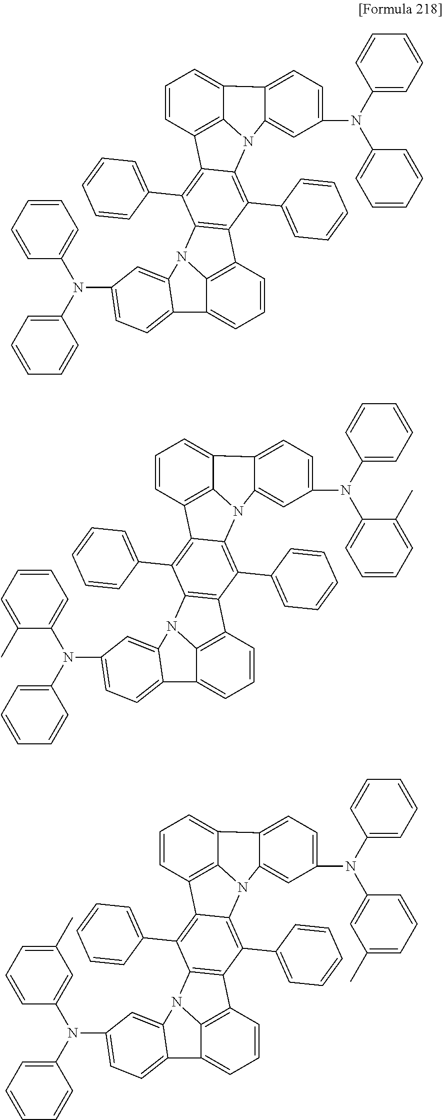

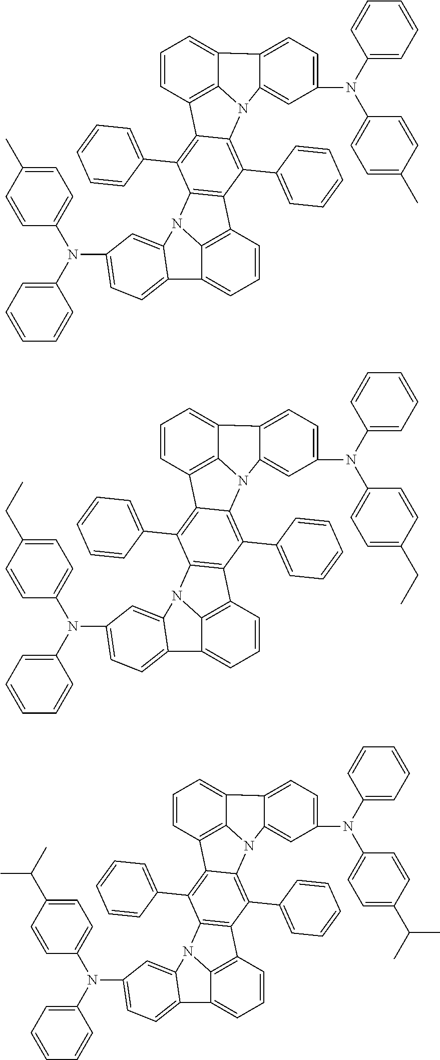

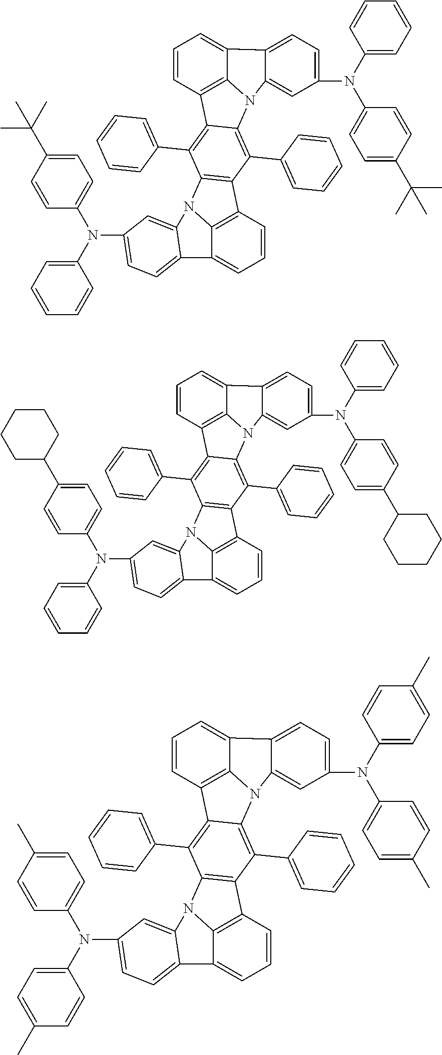

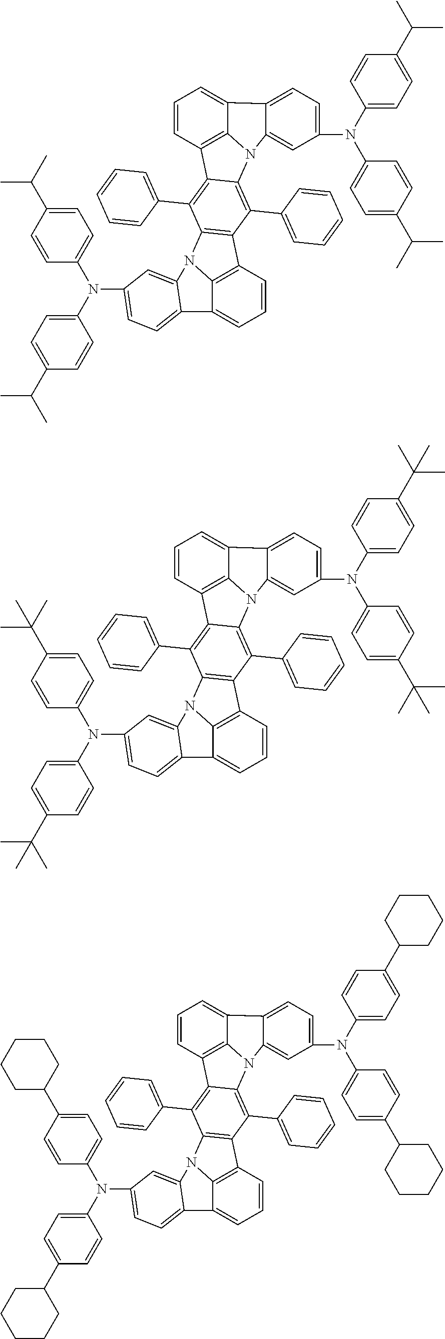

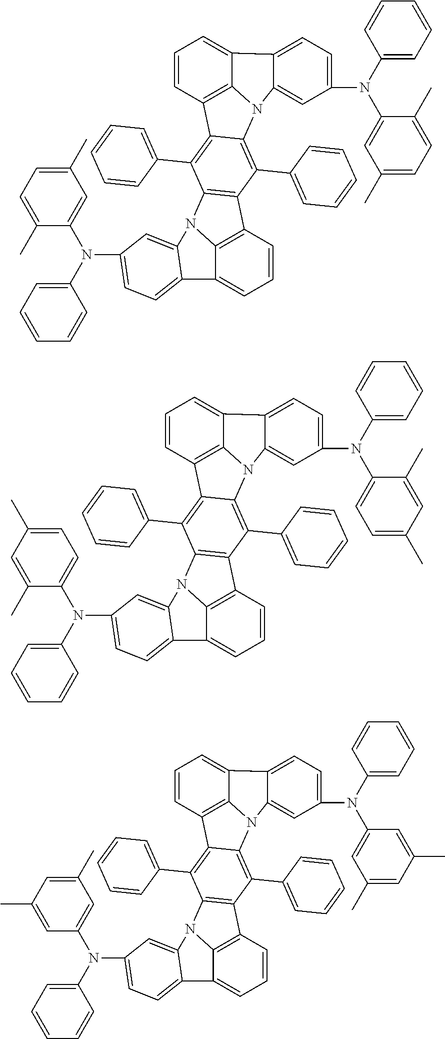

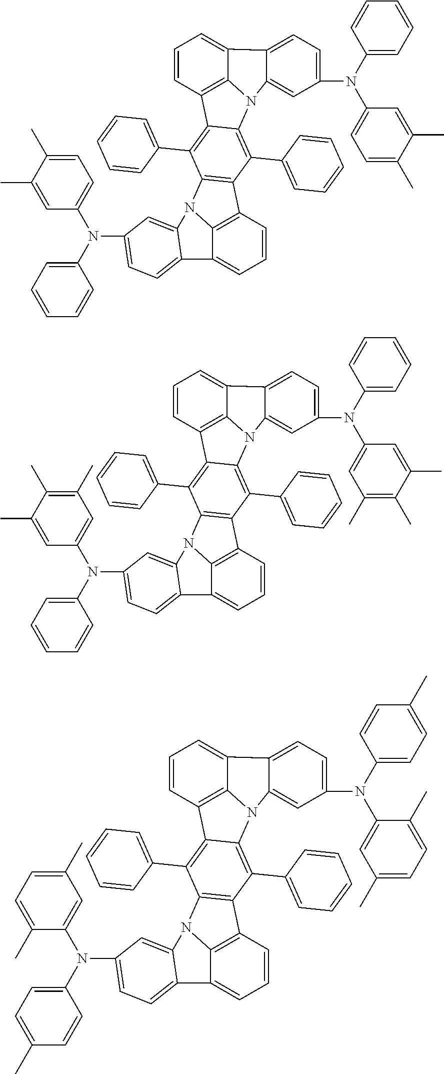

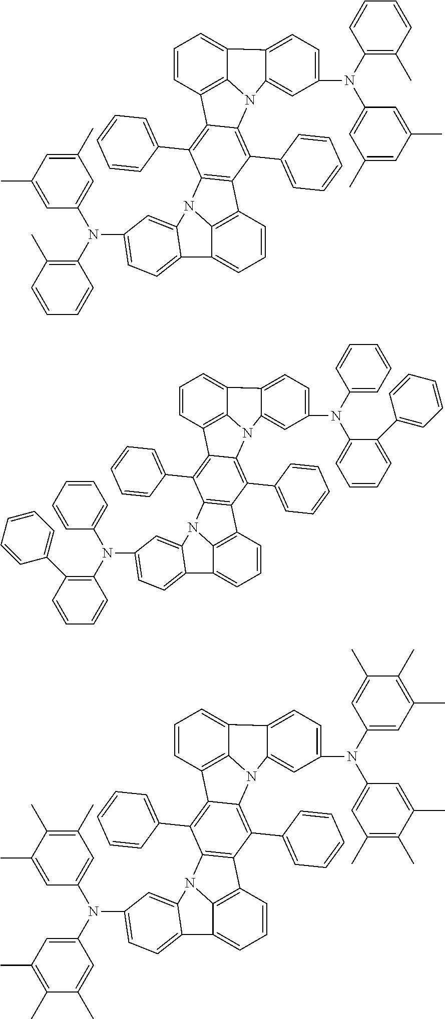

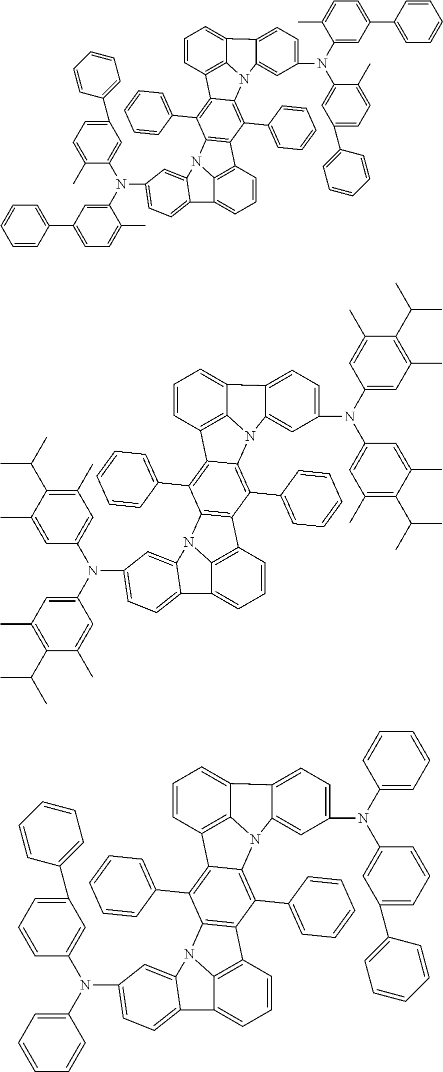

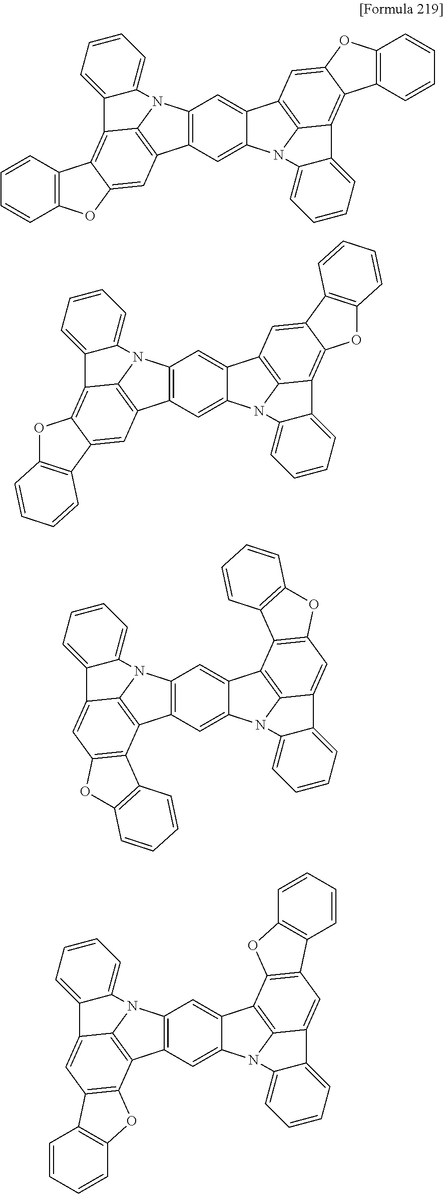

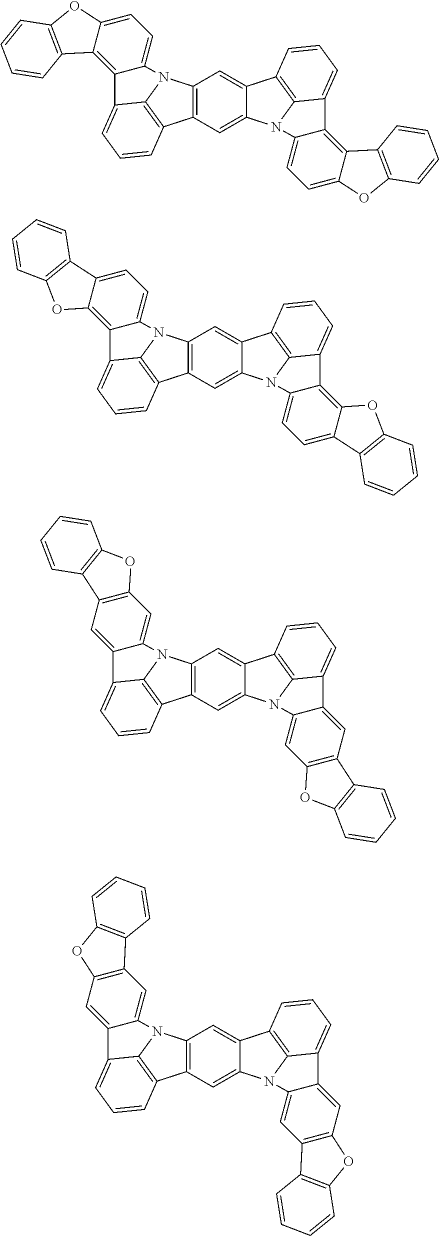

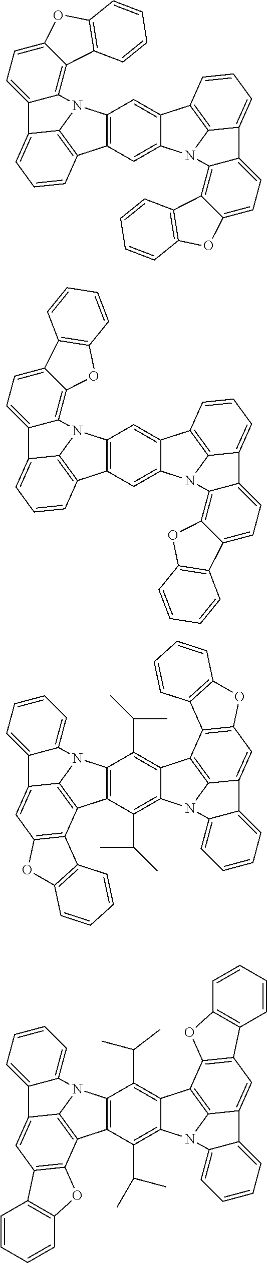

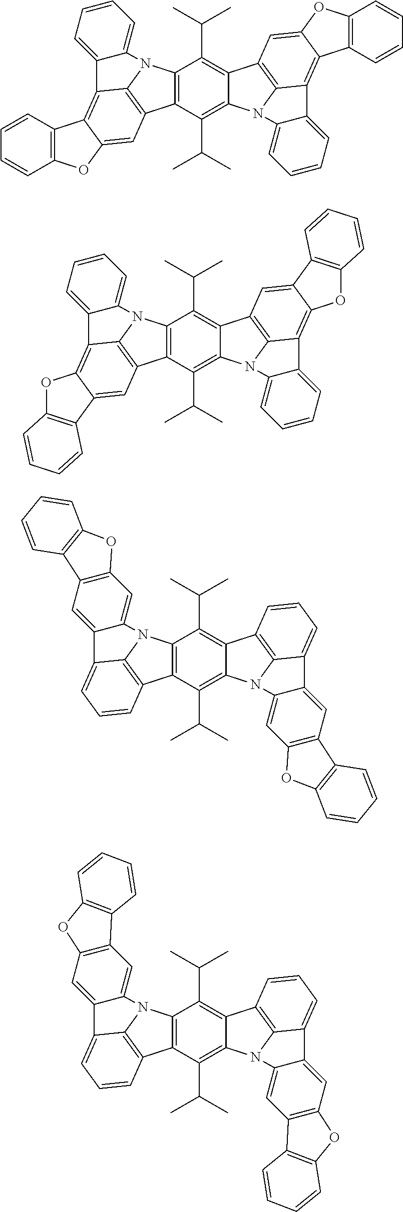

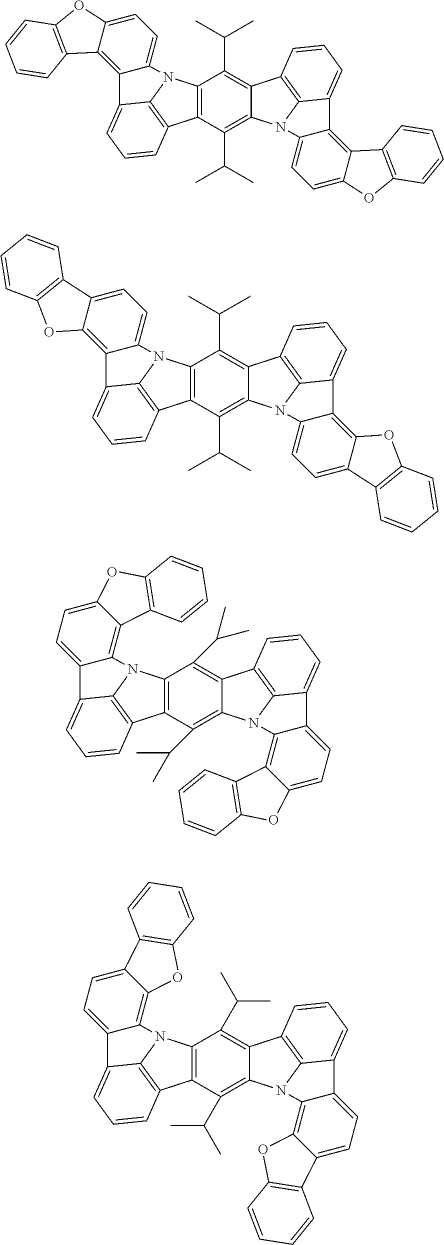

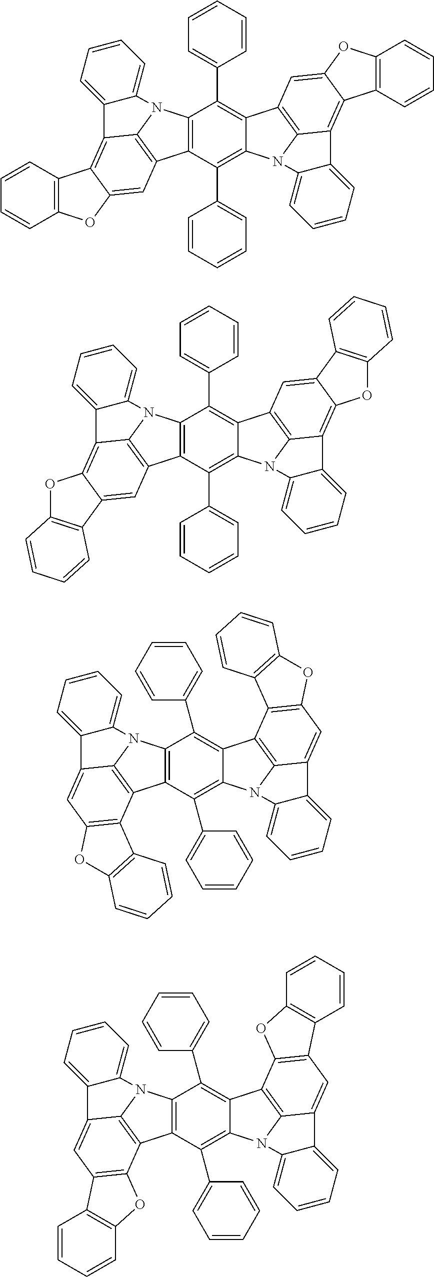

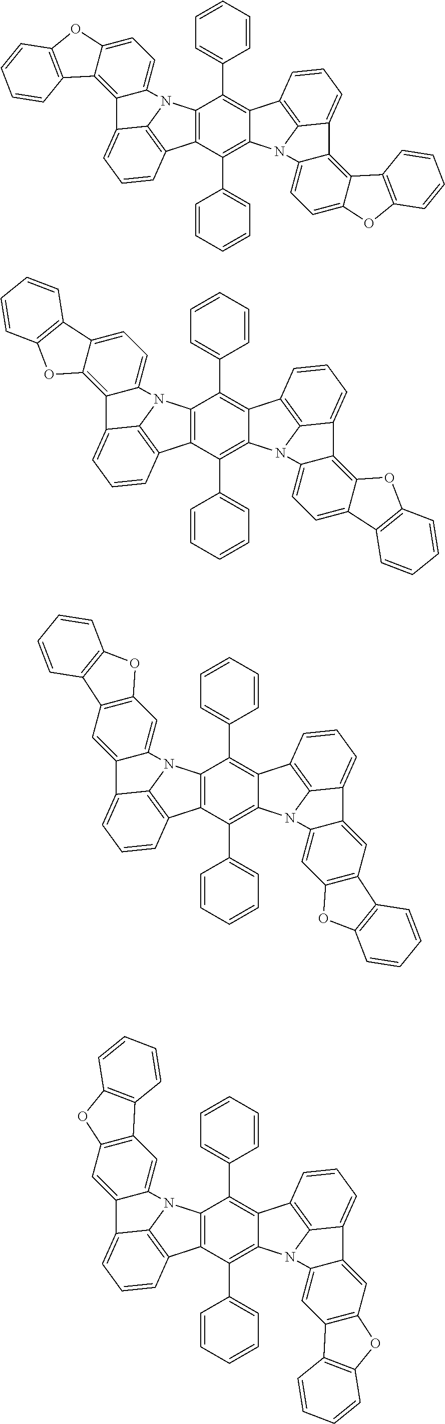

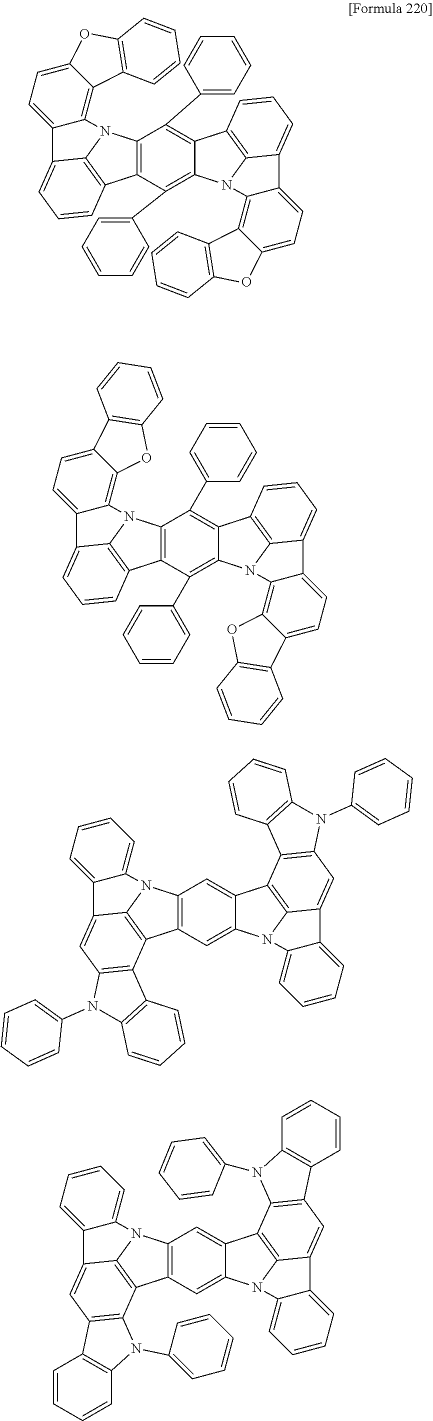

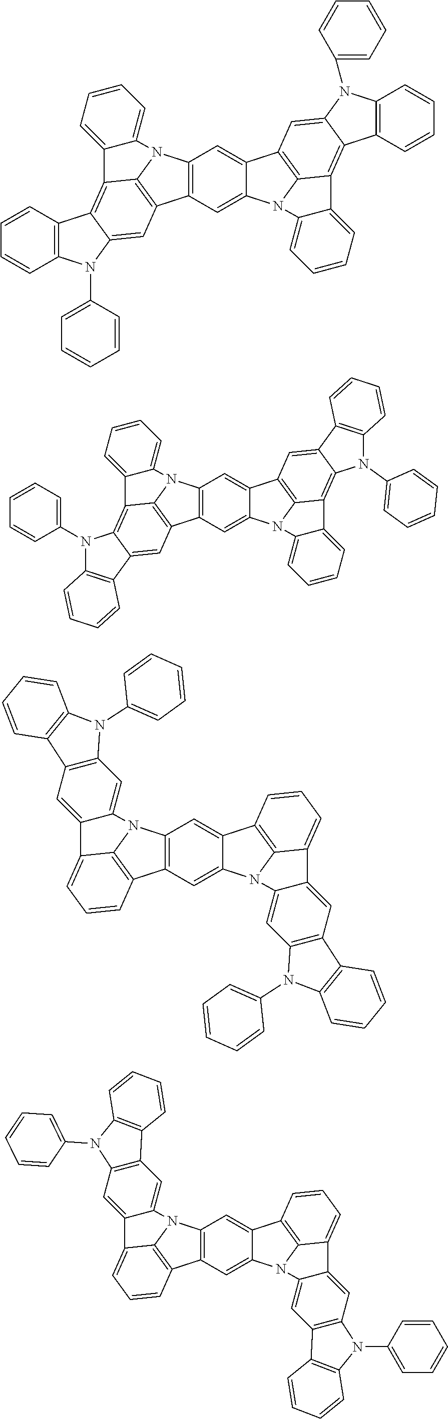

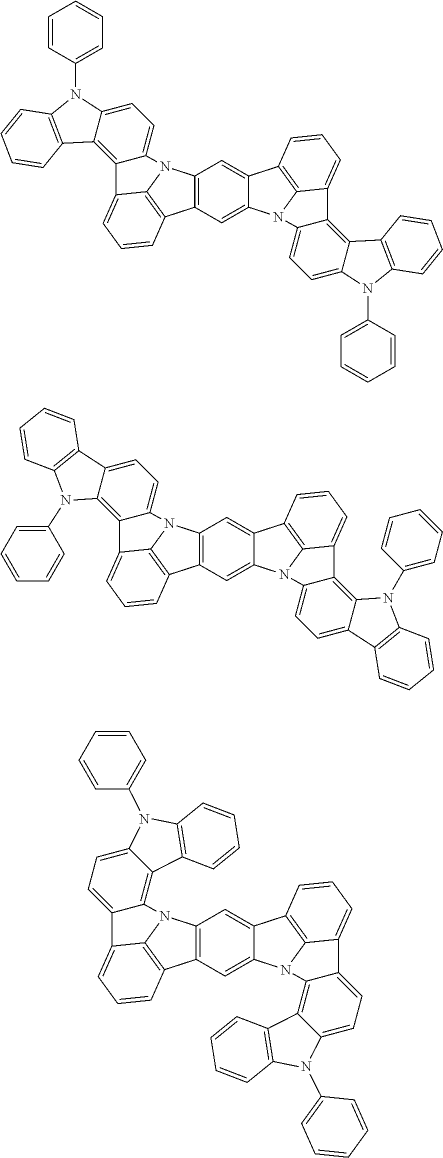

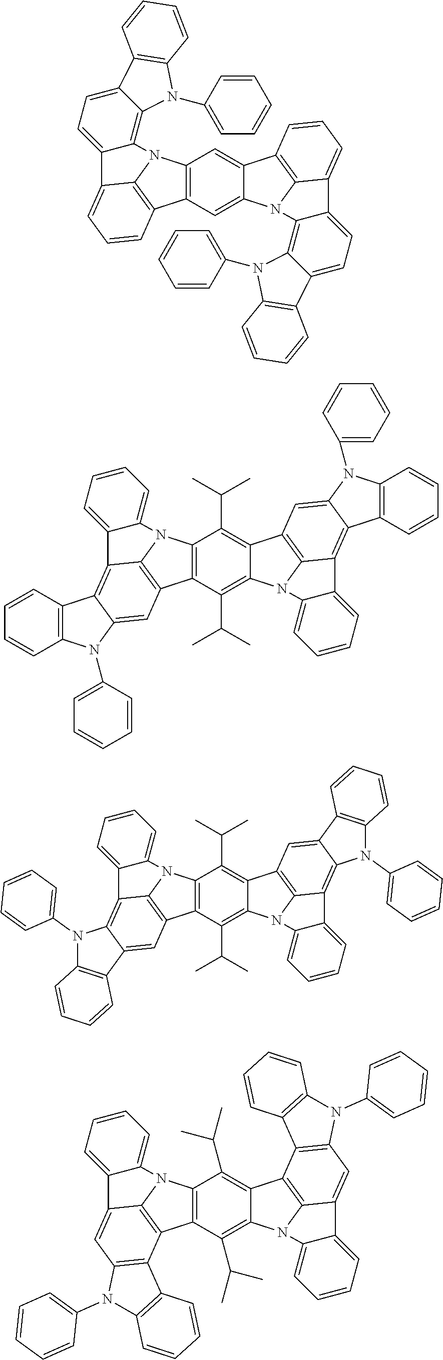

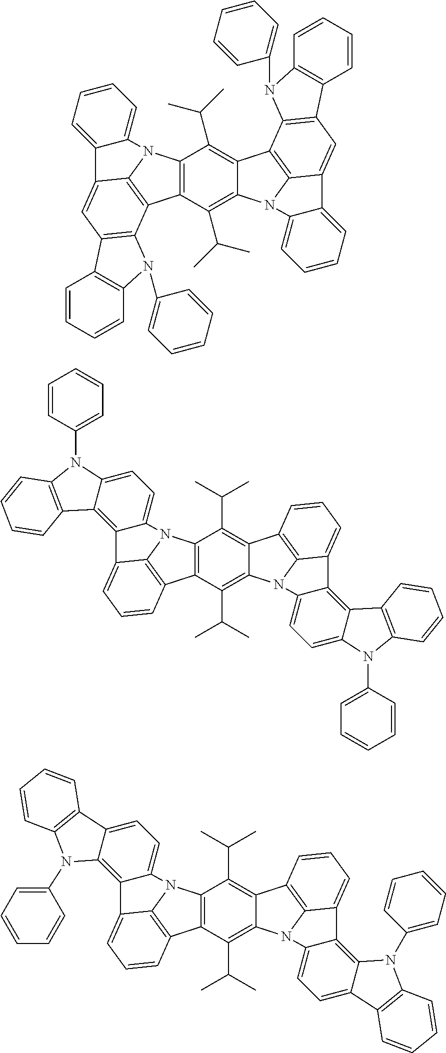

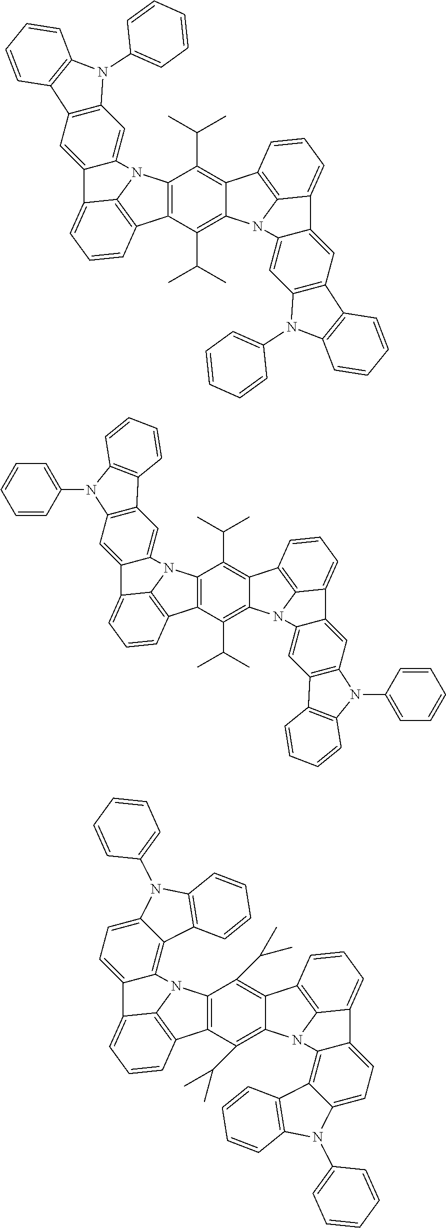

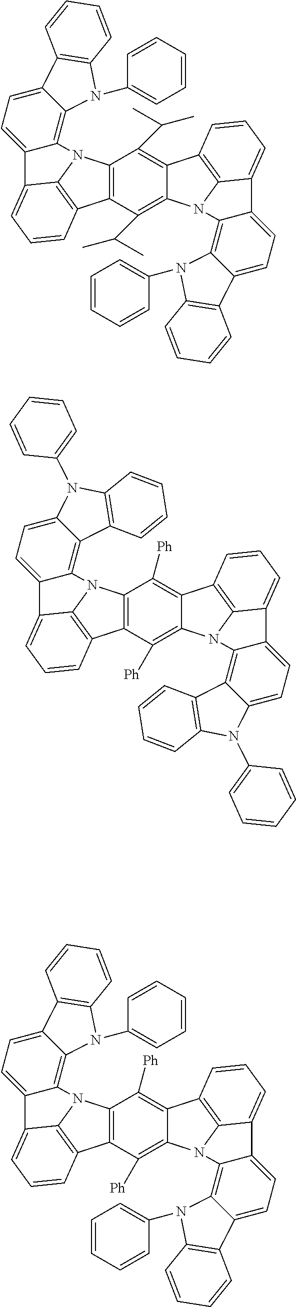

In the formula, Ph is a phenyl group.

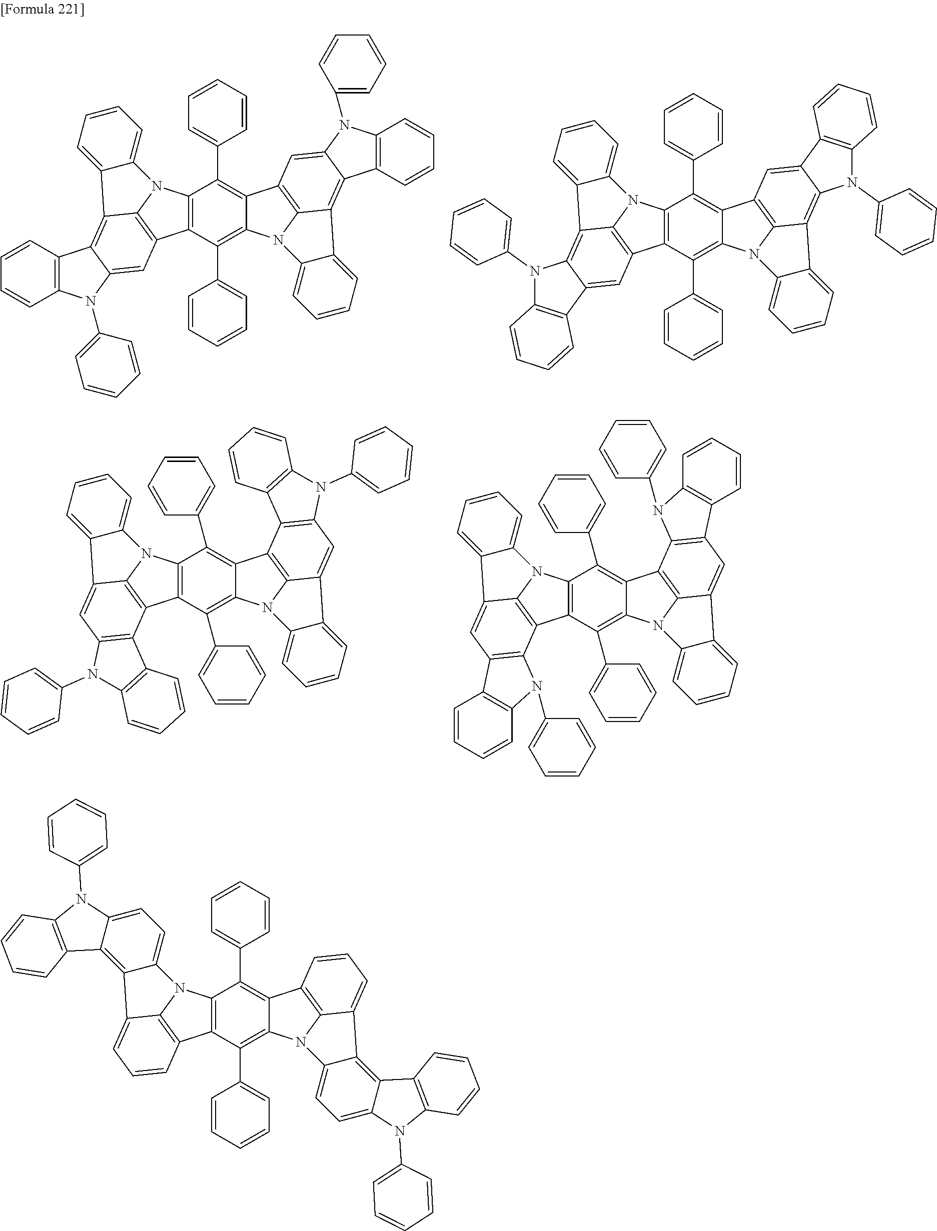

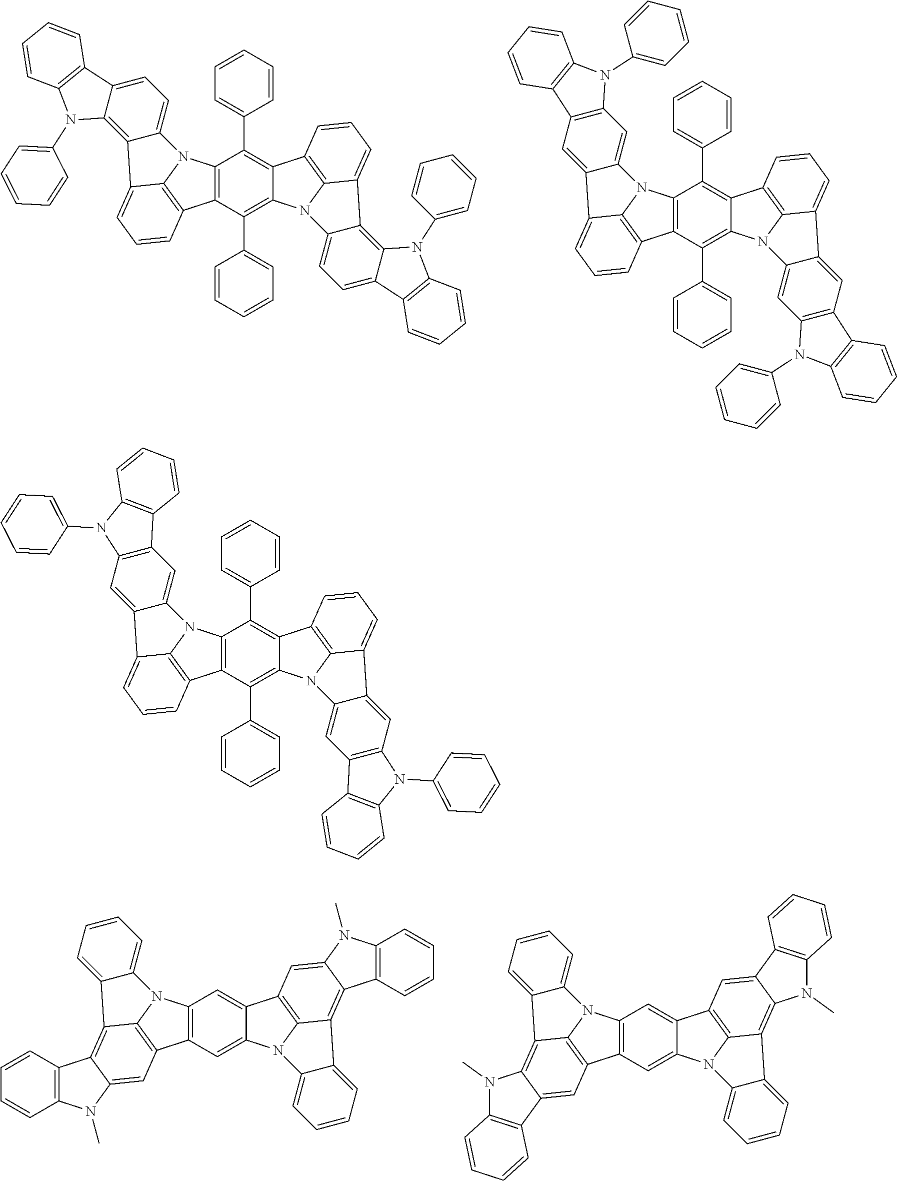

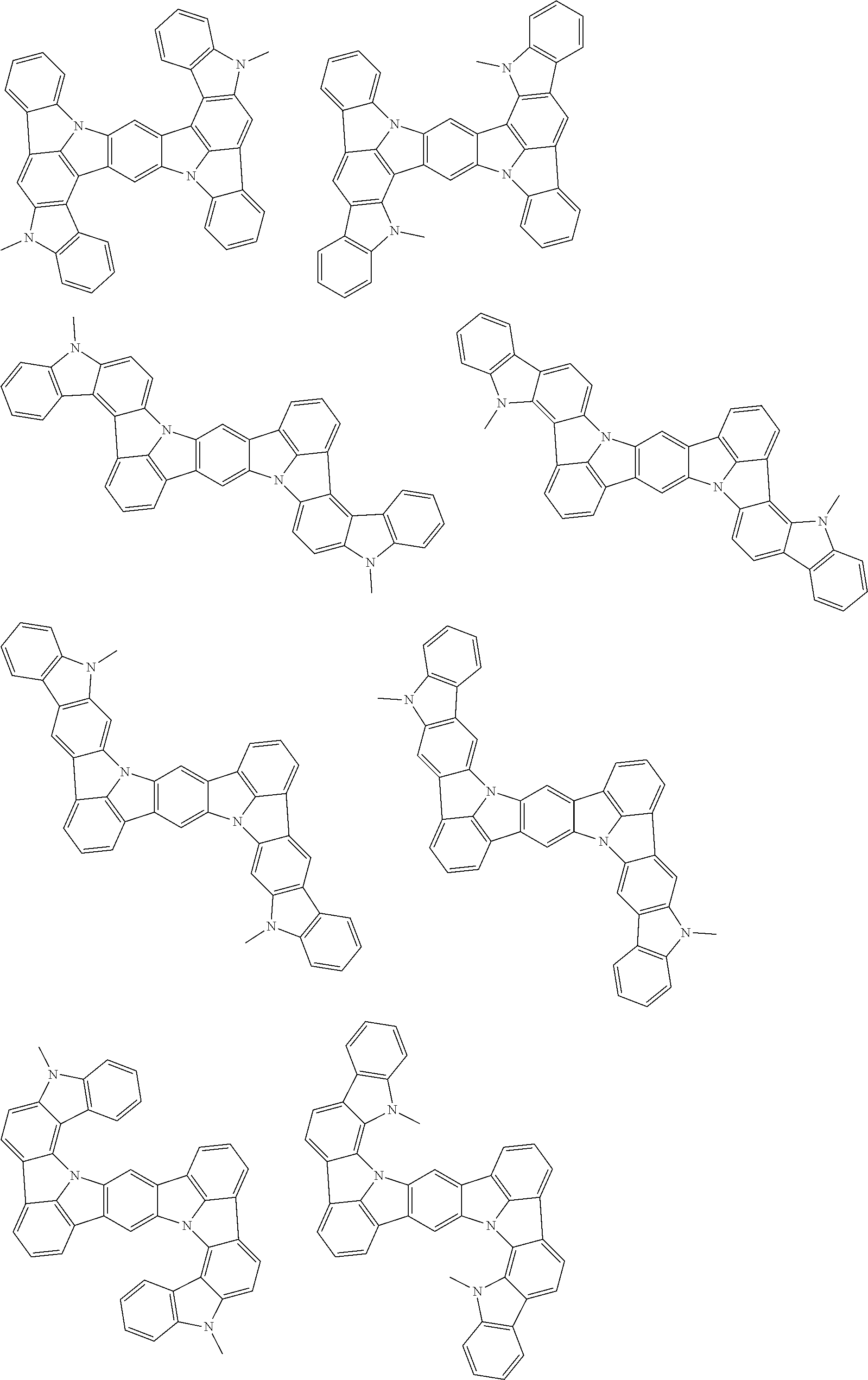

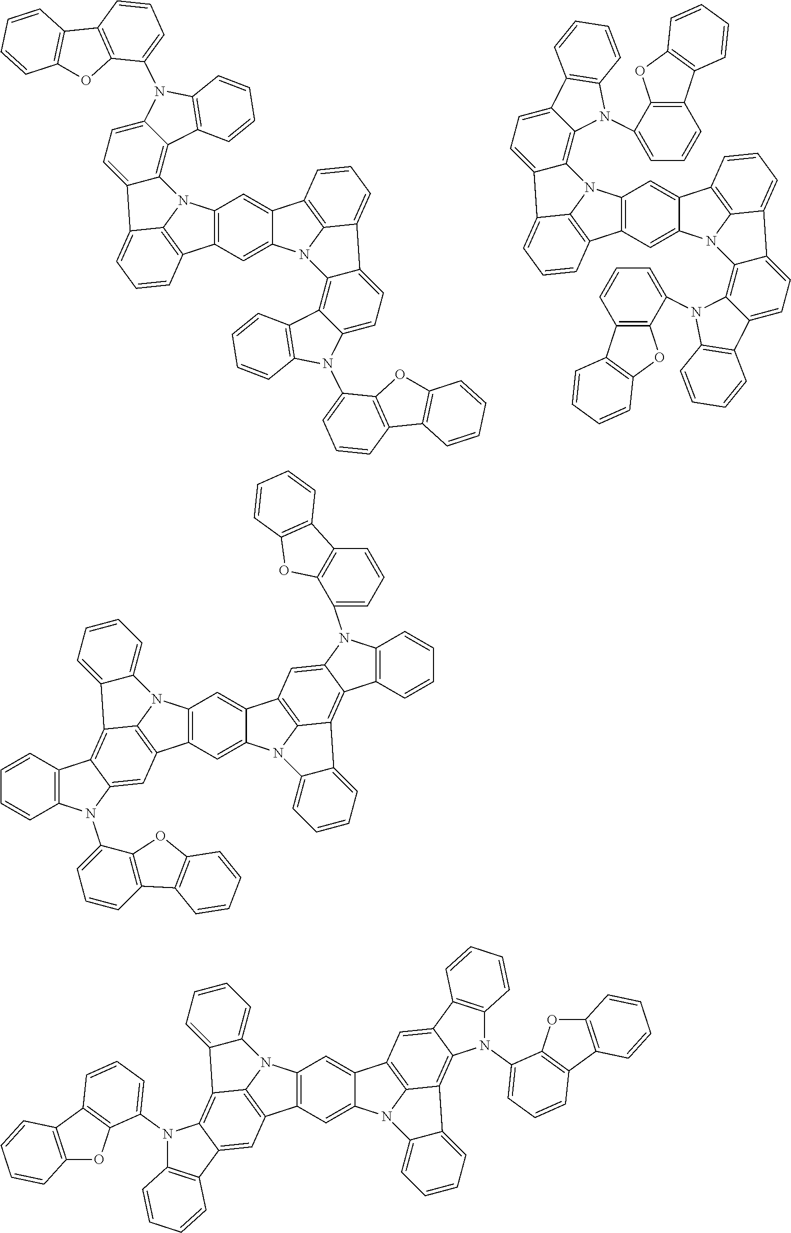

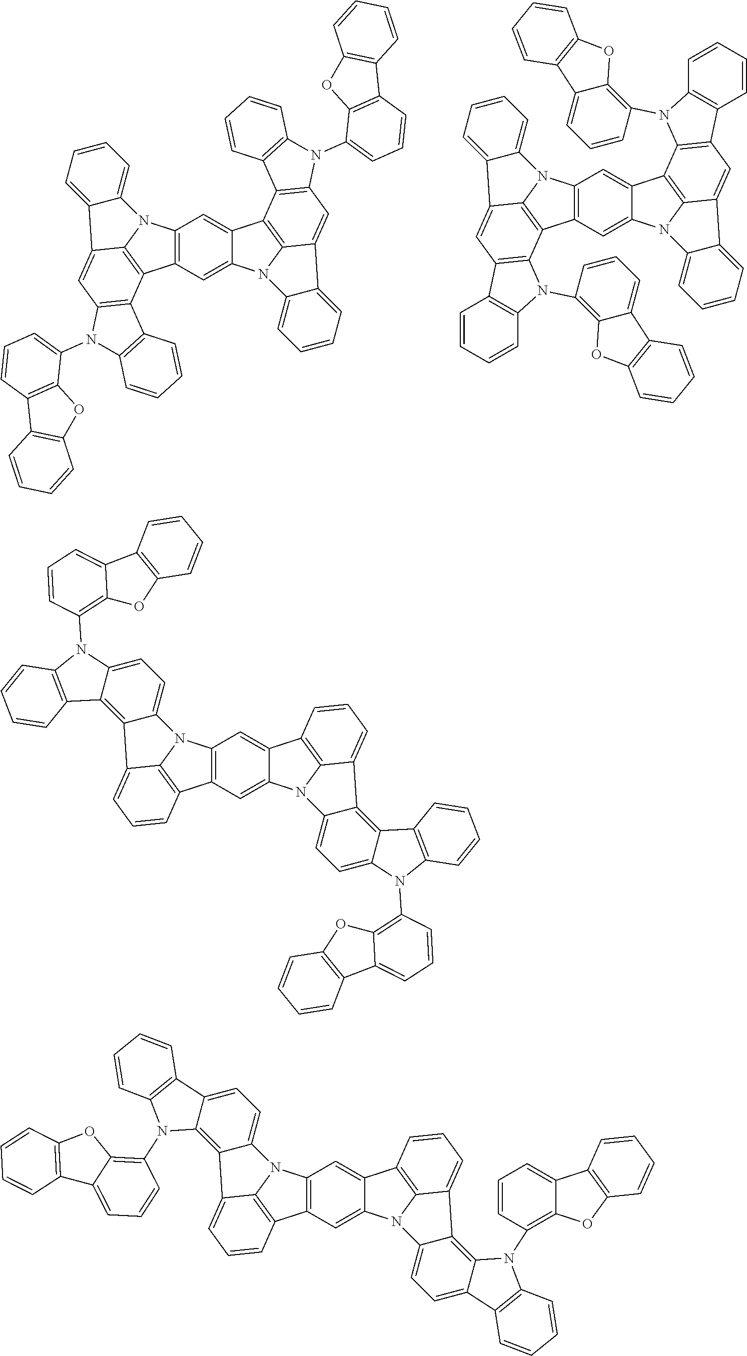

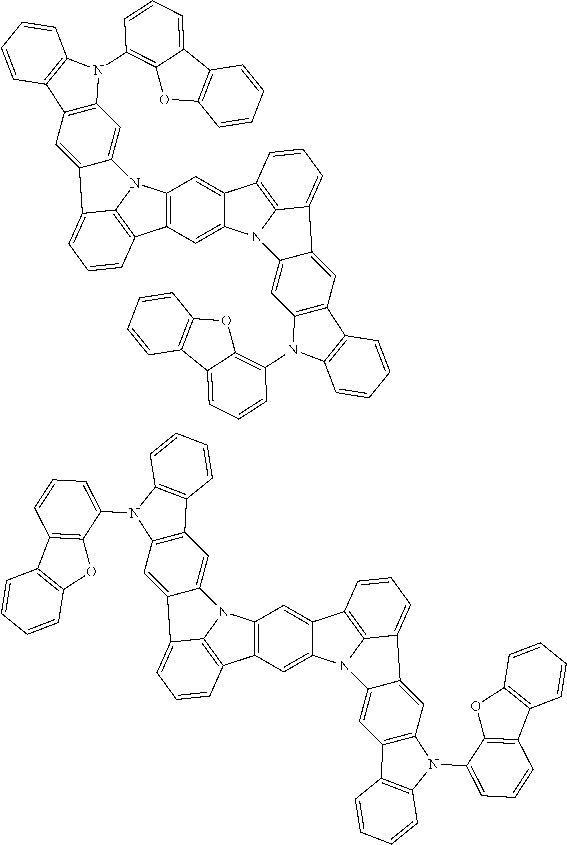

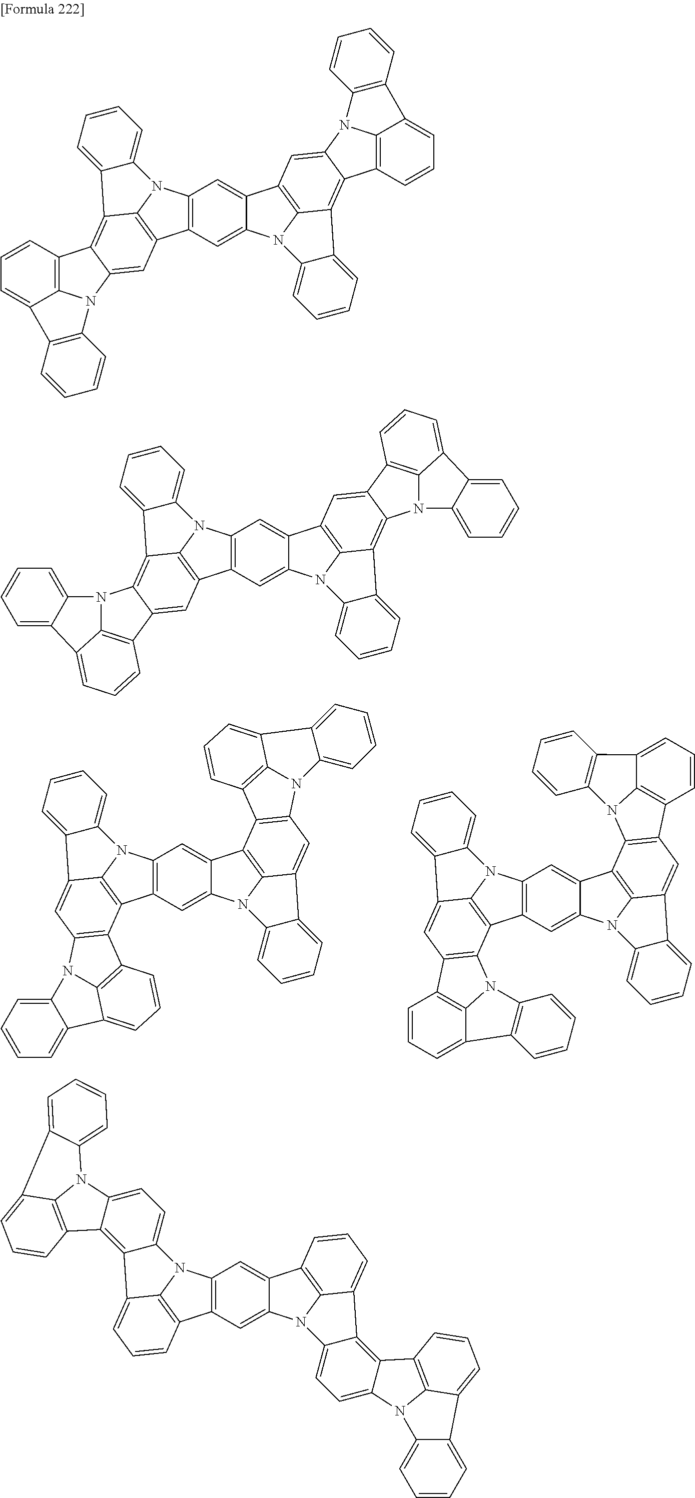

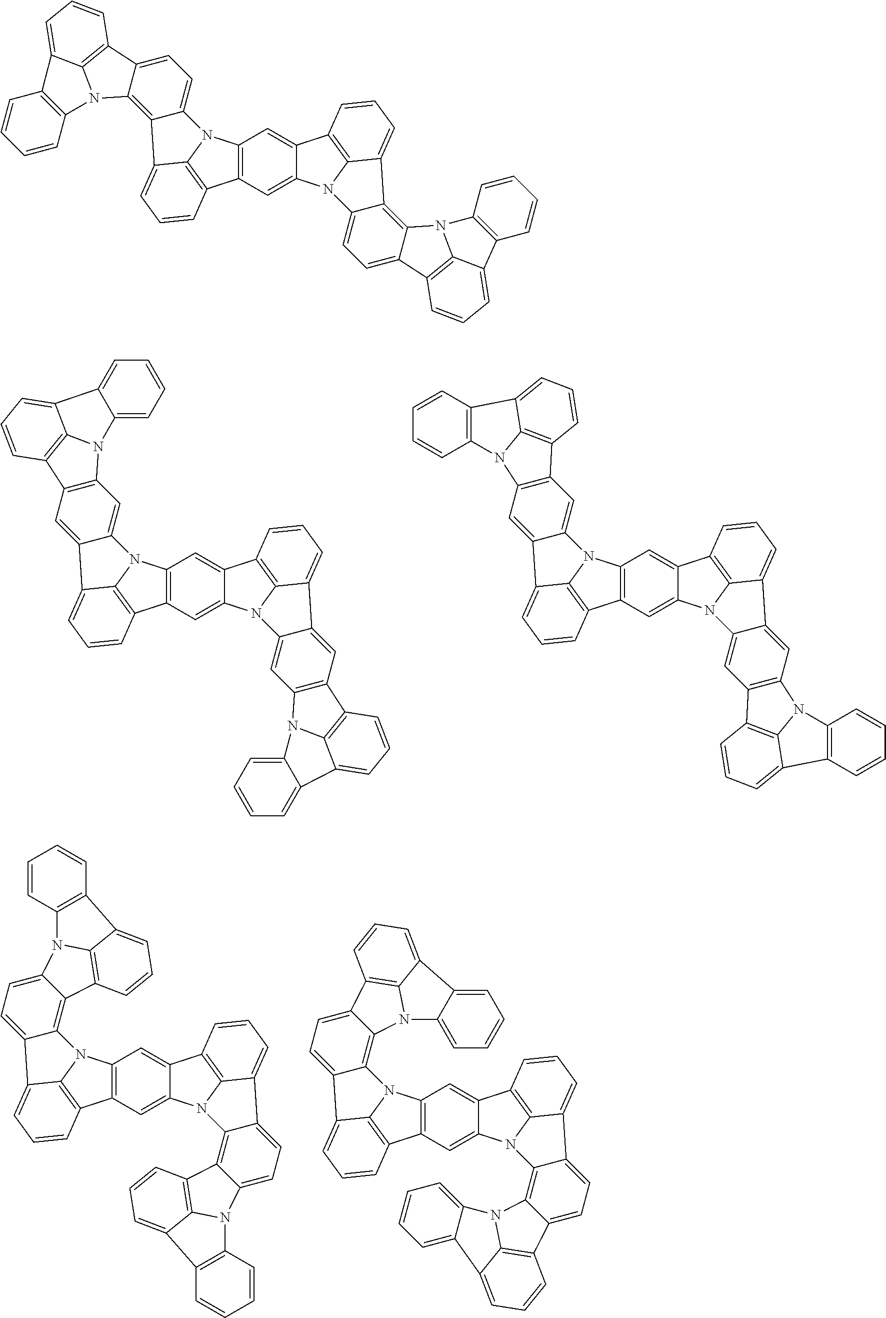

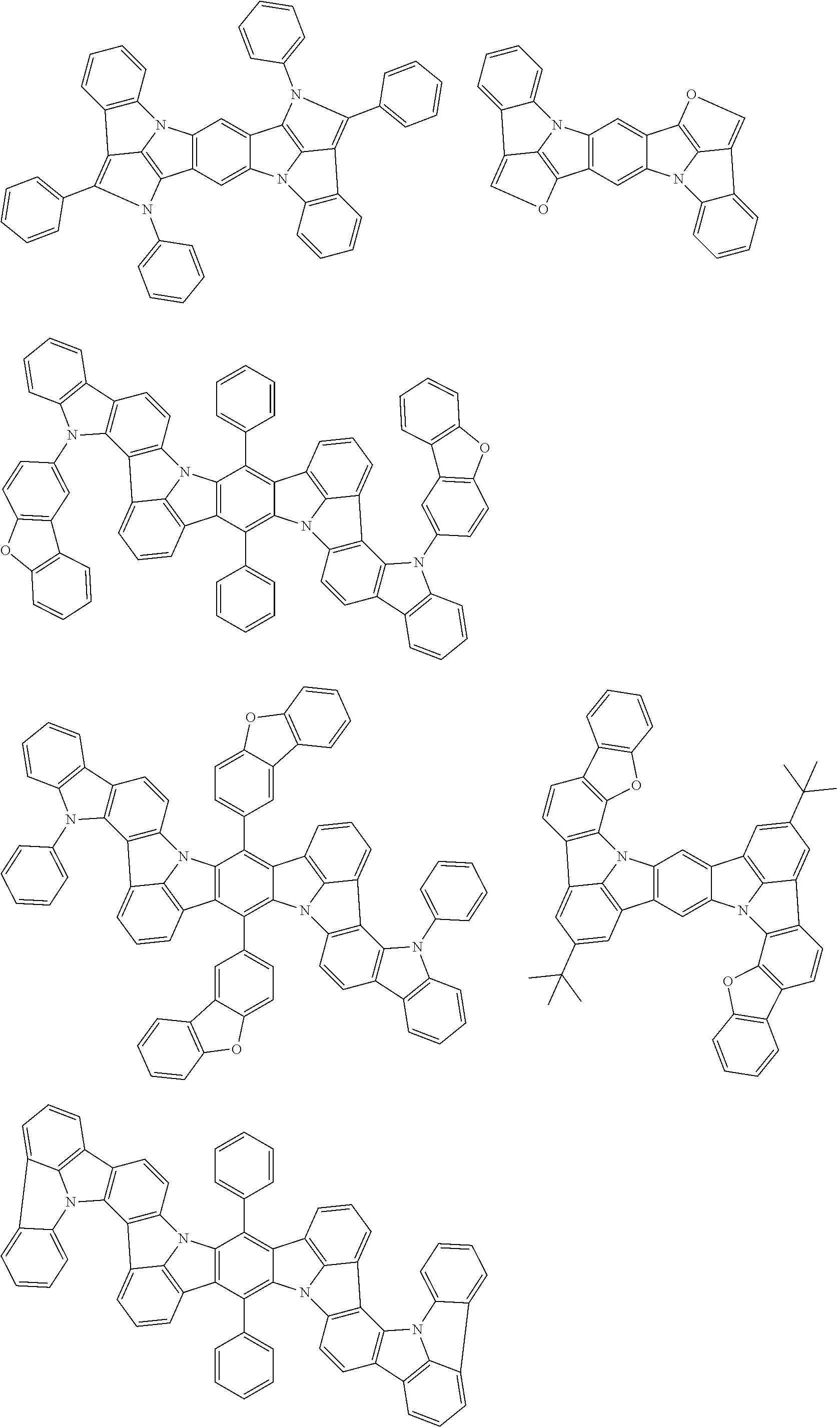

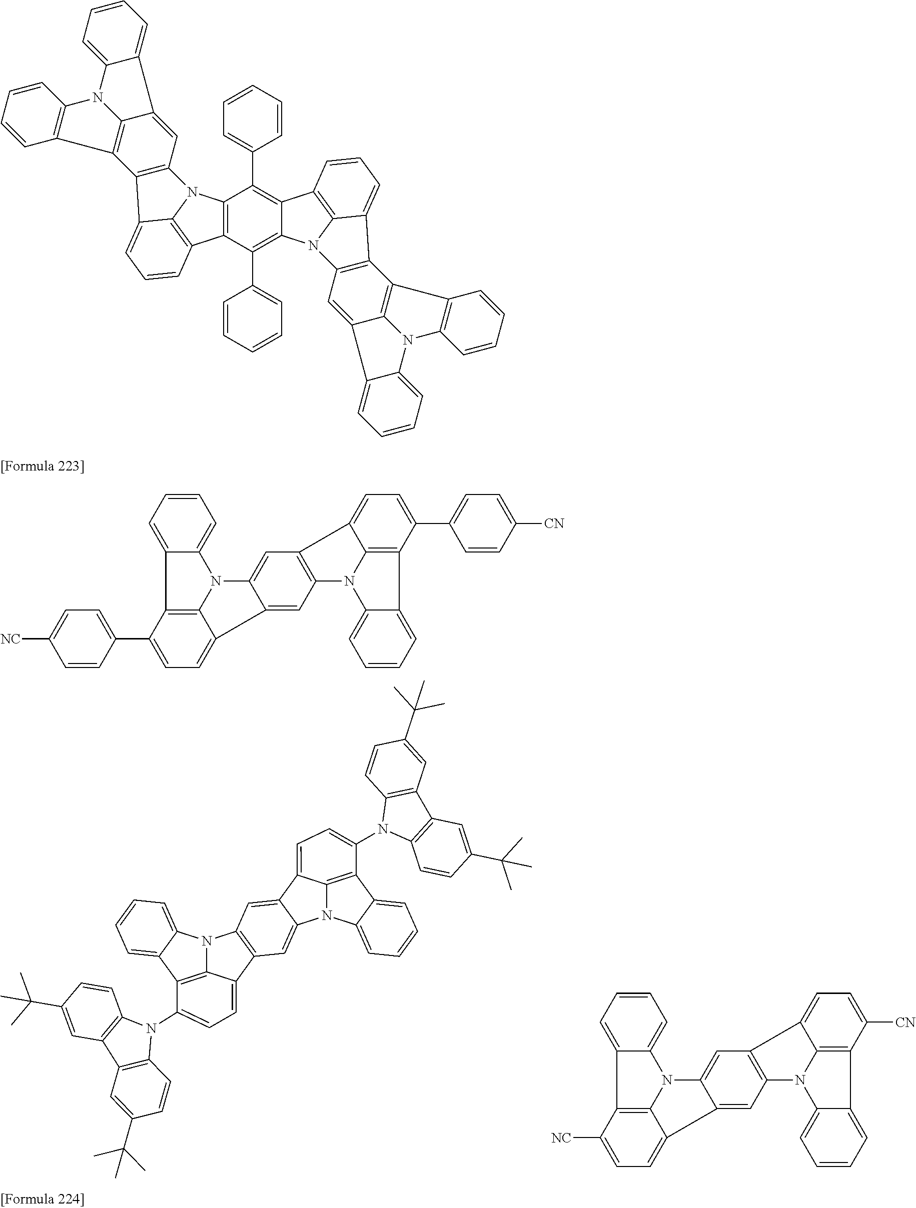

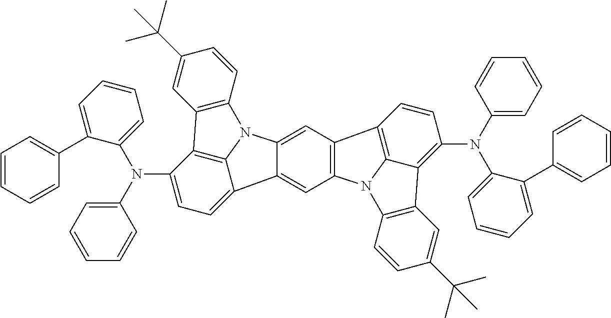 [SMILES: CC(C)(C)c1ccc2c(c1)c1c(N(c3ccccc3)c3ccccc3-c3ccccc3)ccc3c4cc5c(cc4n2c31)c1ccc(N(c2ccccc2)c2ccccc2-c2ccccc2)c2c3cc(C(C)(C)C)ccc3n5c12]

### Compound Represented by Formula (6)

The compound represented by the formula (6) will be described below.

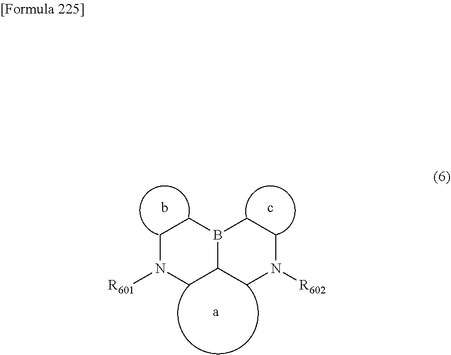

In the formula (6):
a ring a, a ring b and a ring c are each independently a substituted or unsubstituted aromatic hydrocarbon ring having 6 to 50 ring carbon atoms, or a substituted or unsubstituted heterocycle having 5 to 50 ring atoms;
R₆₀₁ and R₆₀₂ are each independently bonded to the ring a, the ring b or the ring c to form a substituted or unsubstituted heterocycle, or not bonded thereto to form no substituted or unsubstituted heterocycle; and
R₆₀₁ and R₆₀₂ not forming the substituted or unsubstituted heterocycle are each independently a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms.

The ring a, ring b and ring c are each a ring (a substituted or unsubstituted aromatic hydrocarbon ring having 6 to 50 ring carbon atoms, or a substituted or unsubstituted heterocycle having 5 to 50 ring atoms) fused with a fused bicyclic structure formed of a boron atom and two nitrogen atoms at the center of the formula (6).

The “aromatic hydrocarbon ring” for the rings a, b, and c has the same structure as a compound formed by introducing a hydrogen atom to the “aryl group” described above.

Ring atoms of the “aromatic hydrocarbon ring” for the ring a include three carbon atoms on the fused bicyclic structure at the center of the formula (6).

Ring atoms of the “aromatic hydrocarbon ring” for the rings b and c include two carbon atoms on the fused bicyclic structure at the center of the formula (6).

Specific examples of the “substituted or unsubstituted aromatic hydrocarbon ring having 6 to 50 ring carbon atoms” include a compound formed by introducing a hydrogen atom to the “aryl group” described in the specific example group G1.

The “heterocycle” for the rings a, b, and c has the same structure as a compound formed by introducing a hydrogen atom to the “heterocyclic group” described above.

Ring atoms of the “heterocycle” for the ring a include three carbon atoms on the fused bicyclic structure at the center of the formula (6). Ring atoms of the “heterocycle” for the rings b and c include two carbon atoms on the fused bicyclic structure at the center of the formula (6). Specific examples of the “substituted or unsubstituted heterocycle having 5 to 50 ring atoms” include a compound formed by introducing a hydrogen atom to the “heterocyclic group” described in the specific example group G2.

R₆₀₁ and R₆₀₂ may be each independently bonded to the ring a, the ring b, or the ring c to form a substituted or unsubstituted heterocycle. The “heterocycle” in this arrangement includes a nitrogen atom on the fused bicyclic structure at the center of the formula (6). The heterocycle in the above arrangement optionally includes a hetero atom other than the nitrogen atom. R₆₀₁ and R₆₀₂ being bonded to the ring a, ring b, or ring c specifically means that atoms forming R₆₀₁ and R₆₀₂ are bonded to atoms forming the ring a, ring b, or ring c. For instance, R₆₀₁ may be bonded with the ring a to form a bicyclic (or tri-or-more cyclic) fused nitrogen-containing heterocycle, in which the ring including R₆₀₁ and the ring a are fused. Specific examples of the nitrogen-containing heterocycle include a compound corresponding to the nitrogen-containing bi (or-more) cyclic fused heterocyclic group in the specific example group G2.

The same applies to R₆₀₁ bonded with the ring b, R₆₀₂ bonded with the ring a, and R₆₀₂ bonded with the ring c.

In an exemplary embodiment, the ring a, ring b and ring c in the formula (6) are each independently a substituted or unsubstituted aromatic hydrocarbon ring having 6 to 50 ring carbon atoms.

In an exemplary embodiment, the ring a, ring b and ring c in the formula (6) are each independently a substituted or unsubstituted benzene ring or a substituted or unsubstituted naphthalene ring.

In an exemplary embodiment, R₆₀₁ and R₆₀₂ in the formula (6) are each independently a substituted or unsubstituted aryl group having 6 to 50 ring carbon atoms, or a substituted or unsubstituted heterocyclic group having 5 to 50 ring atoms; preferably a substituted or unsubstituted aryl group having 6 to 50 ring carbon atoms.

In an exemplary embodiment, the compound represented by the formula (6) is a compound represented by a formula (62) below.

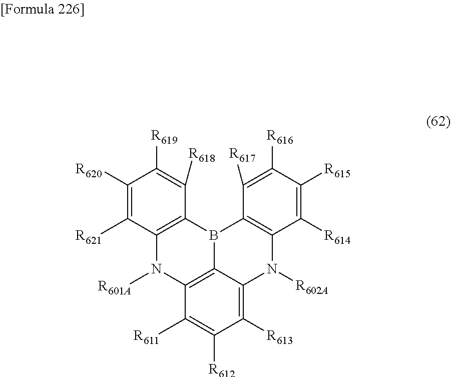

In the formula (62):
R_{601A} is bonded with at least one of R₆₁₁ or R₆₂₁ to form a substituted or unsubstituted heterocycle, or not bonded therewith to form no substituted or unsubstituted heterocycle;
R_{602A} is bonded with at least one of R₆₁₃ or R₆₁₄ to form a substituted or unsubstituted heterocycle, or not bonded therewith to form no substituted or unsubstituted heterocycle;
R_{601A} and R_{602A} not forming the substituted or unsubstituted heterocycle are each independently a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms;
at least one combination of adjacent two or more of R₆₁₁ to R₆₂₁ are mutually bonded to form a substituted or unsubstituted monocyclic ring, mutually bonded to form a substituted or unsubstituted fused ring, or not mutually bonded; and
R₆₁₁ to R₆₂₁ forming neither the substituted or unsubstituted heterocycle nor the monocyclic ring nor the fused ring are each independently a hydrogen atom, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a halogen atom, a cyano group, a nitro group, a substituted or unsubstituted aryl group having 6 to 50 ring carbon atoms, or a substituted or unsubstituted heterocyclic group having 5 to 50 ring atoms.
R_{601A} and R_{602A} in the formula (62) are groups corresponding to R₆₀₁ and R₆₀₂ in the formula (6), respectively.

For instance, R_{601A} and R₆₁₁ are optionally bonded with each other to form a bicyclic (or tri-or-more cyclic) fused nitrogen-containing heterocycle, in which the ring including R_{601A} and R₆₁₁ and a benzene ring corresponding to the ring a are fused. Specific examples of the nitrogen-containing heterocycle include a compound corresponding to the nitrogen-containing bi (or-more) cyclic fused heterocyclic group in the specific example group G2. The same applies to R_{601A} bonded with R₆₂₁, R_{602A} bonded with R₆₁₃, and R_{602A} bonded with R₆₁₄.

At least one combination of adjacent two or more of R₆₁₁ to R₆₂₁ are mutually bonded to form a substituted or unsubstituted monocyclic ring, or mutually bonded to form a substituted or unsubstituted fused ring.

For instance, R₆₁₁ and R₆₁₂ are optionally mutually bonded to form a structure in which a benzene ring, indole ring, pyrrole ring, benzofuran ring, benzothiophene ring or the like is fused to the six-membered ring bonded with R₆₁₁ and R₆₁₂, the resultant fused ring forming a naphthalene ring, carbazole ring, indole ring, dibenzofuran ring, or dibenzothiophene ring.

In an exemplary embodiment, R₆₁₁ to R₆₂₁ not contributing to ring formation are each independently a hydrogen atom, a substituted or unsubstituted alkyl group having 1 to 50 carbon atoms, a substituted or unsubstituted aryl group having 6 to 50 ring carbon atoms, or a substituted or unsubstituted heterocyclic group having 5 to 50 ring atoms.

In an exemplary embodiment, R₆₁₁ to R₆₂₁ not contributing to ring formation are each independently a hydrogen atom, a substituted or unsubstituted aryl group having 6 to 50 ring carbon atoms, or a substituted or unsubstituted heterocyclic group having 5 to 50 ring atoms.

In an exemplary embodiment, R₆₁₁ to R₆₂₁ not contributing to ring formation are each independently a hydrogen atom, or a substituted or unsubstituted alkyl group having 1 to 50 carbon atoms.

In an exemplary embodiment, R₆₁₁ to R₆₂₁ not contributing to ring formation are each independently a hydrogen atom, or a substituted or unsubstituted alkyl group having 1 to 50 carbon atoms, and
at least one of R₆₁₁ to R₆₂₁ is a substituted or unsubstituted alkyl group having 1 to 50 carbon atoms.

In an exemplary embodiment, the compound represented by the formula (62) is a compound represented by a formula (63) below.

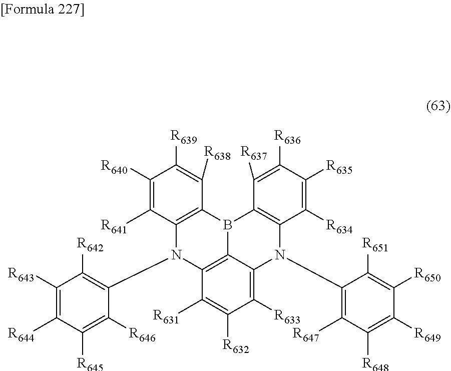

In the formula (63):
R₆₃₁ is bonded with R₆₄₆ to form a substituted or unsubstituted heterocycle, or not bonded therewith to form no substituted or unsubstituted heterocycle;
R₆₃₃ is bonded with R₆₄₇ to form a substituted or unsubstituted heterocycle, or not bonded therewith to form no substituted or unsubstituted heterocycle;
R₆₃₄ is bonded with R₆₅₁ to form a substituted or unsubstituted heterocycle, or not bonded therewith to form no substituted or unsubstituted heterocycle;
R₆₄₁ is bonded with R₆₄₂ to form a substituted or unsubstituted heterocycle, or not bonded therewith to form no substituted or unsubstituted heterocycle;
at least one combination of adjacent two or more of R₆₃₁ to R₆₅₁ are mutually bonded to form a substituted or unsubstituted monocyclic ring, mutually bonded to form a substituted or unsubstituted fused ring, or not mutually bonded; and
R₆₃₁ to R₆₅₁ forming neither the substituted or unsubstituted heterocycle nor the monocyclic ring nor the fused ring are each independently a hydrogen atom, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a halogen atom, a cyano group, a nitro group, a substituted or unsubstituted aryl group having 6 to 50 ring carbon atoms, or a substituted or unsubstituted heterocyclic group having 5 to 50 ring atoms.

R₆₃₁ is optionally bonded with R₆₄₆ to form a substituted or unsubstituted heterocycle. For instance, R₆₃₁ and R₆₄₆ are optionally bonded with each other to form a tri-or-more cyclic fused nitrogen-containing heterocycle, in which a benzene ring bonded with R₆₄₆, a ring including a nitrogen atom, and a benzene ring corresponding to the ring a are fused. Specific examples of the nitrogen-containing heterocycle include a compound corresponding to a nitrogen-containing tri (-or-more) cyclic fused heterocyclic group in the specific example group G2. The same applies to R₆₃₃ bonded with R₆₄₇, R₆₃₄ bonded with R₆₅₁, and R₆₄₁ bonded with R₆₄₂.

In an exemplary embodiment, R₆₃₁ to R₆₅₁ not contributing to ring formation are each independently a hydrogen atom, a substituted or unsubstituted alkyl group having 1 to 50 carbon atoms, a substituted or unsubstituted aryl group having 6 to 50 ring carbon atoms, or a substituted or unsubstituted heterocyclic group having 5 to 50 ring atoms.

In an exemplary embodiment, R₆₃₁ to R₆₅₁ not contributing to ring formation are each independently a hydrogen atom, a substituted or unsubstituted aryl group having 6 to 50 ring carbon atoms, or a substituted or unsubstituted heterocyclic group having 5 to 50 ring atoms.

In an exemplary embodiment, R₆₃₁ to R₆₅₁ not contributing to ring formation are each independently a hydrogen atom, or a substituted or unsubstituted alkyl group having 1 to 50 carbon atoms.

In an exemplary embodiment, R₆₃₁ to R₆₅₁ not contributing to ring formation are each independently a hydrogen atom, or a substituted or unsubstituted alkyl group having 1 to 50 carbon atoms, and
at least one of R₆₃₁ to R₆₅₁ is a substituted or unsubstituted alkyl group having 1 to 50 carbon atoms.

In an exemplary embodiment, the compound represented by the formula (63) is a compound represented by a formula (63A) below.

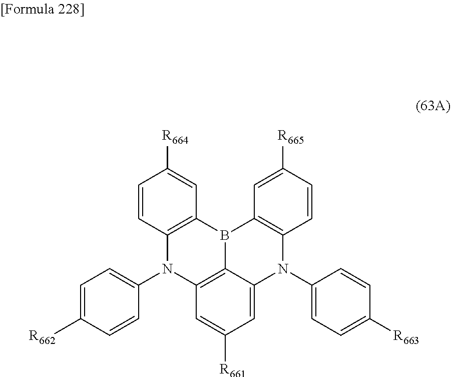

In the formula (63A):
R₆₆₁ is a hydrogen atom, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, or a substituted or unsubstituted aryl group having 6 to 50 ring carbon atoms; and
R₆₆₂ to R₆₆₅ are each independently a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, or a substituted or unsubstituted aryl group having 6 to 50 ring carbon atoms.

In an exemplary embodiment, R₆₆₁ to R₆₆₅ are each independently a substituted or unsubstituted alkyl group having 1 to 50 carbon atoms, or a substituted or unsubstituted aryl group having 6 to 50 ring carbon atoms.

In an exemplary embodiment, R₆₆₁ to R₆₆₅ are each independently a substituted or unsubstituted alkyl group having 1 to 50 carbon atoms.

In an exemplary embodiment, the compound represented by the formula (63) is a compound represented by a formula (63B) below.

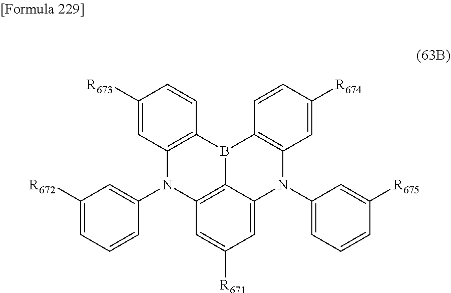

In the formula (63B):
R₆₇₁ and R₆₇₂ are each independently a hydrogen atom, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —N(R₉₀₆)(R₉₀₇), or a substituted or unsubstituted aryl group having 6 to 50 ring carbon atoms; and
R₆₇₃ to R₆₇₅ are each independently a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —N(R₉₀₆)(R₉₀₇), or a substituted or unsubstituted aryl group having 6 to 50 ring carbon atoms.

In an exemplary embodiment, the compound represented by the formula (63) is a compound represented by a formula (63B′) below.

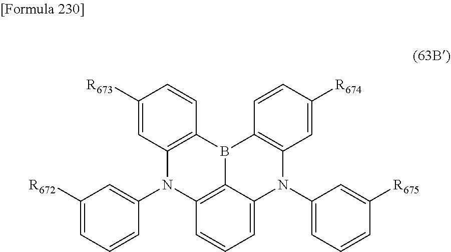

In the formula (63B′), R₆₇₂ to R₆₇₅ each independently represent the same as R₆₇₂ to R₆₇₅ in the formula (63B).

In an exemplary embodiment, at least one of R₆₇₁ to R₆₇₅ is a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —N(R₉₀₆)(R₉₀₇), or a substituted or unsubstituted aryl group having 6 to 50 ring carbon atoms.

In an exemplary embodiment:
R₆₇₂ is a hydrogen atom, a substituted or unsubstituted alkyl group having 1 to 50 carbon atoms, a group represented by —N(R₉₀₆)(R₉₀₇), or a substituted or unsubstituted aryl group having 6 to 50 ring carbon atoms; and
R₆₇₁ and R₆₇₃ to R₆₇₅ are each independently a substituted or unsubstituted alkyl group having 1 to 50 carbon atoms, a group represented by —N(R₉₀₆)(R₉₀₇), or a substituted or unsubstituted aryl group having 6 to 50 ring carbon atoms.

In an exemplary embodiment, the compound represented by the formula (63) is a compound represented by a formula (63C) below.

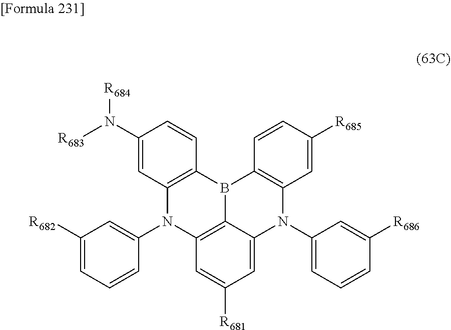

In the formula (63C):
R₆₈₁ and R₆₈₂ are each independently a hydrogen atom, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, or a substituted or unsubstituted aryl group having 6 to 50 ring carbon atoms; and
R₆₈₃ to R₆₈₆ are each independently a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, or a substituted or unsubstituted aryl group having 6 to 50 ring carbon atoms.

In an exemplary embodiment, the compound represented by the formula (63) is a compound represented by a formula (63C′) below.

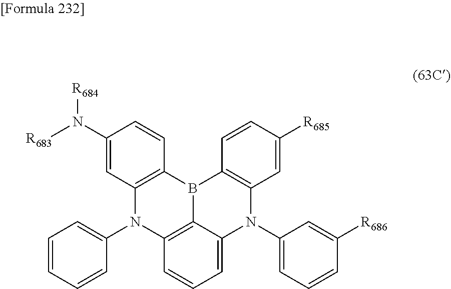

In the formula (63C′), R₆₈₃ to R₆₈₆ each independently represent the same as R₆₈₃ to R₆₈₆ in the formula (63C).

In an exemplary embodiment, R₆₈₁ to R₆₈₆ are each independently a substituted or unsubstituted alkyl group having 1 to 50 carbon atoms, or a substituted or unsubstituted aryl group having 6 to 50 ring carbon atoms.

In an exemplary embodiment, R₆₈₁ to R₆₈₆ are each independently a substituted or unsubstituted aryl group having 6 to 50 carbon atoms.

In an exemplary embodiment, the compound represented by the formula (6) is also preferably a compound represented by a formula (64) below.

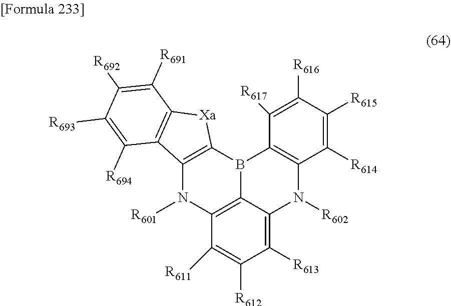

In the formula (64):
Xa is O, S, Se, C(R₆₀₃)(R₆₀₄), or NR₆₀₅;
at least one combination selected from the group consisting of a combination of R₆₀₁ and R₆₁₁, a combination of adjacent two or more of R₆₁₁ to R₆₁₃, a combination of R₆₁₃ and R₆₀₂, a combination of R₆₀₂ and R₆₁₄, a combination of adjacent two or more of R₆₁₄ to R₆₁₇, and a combination of adjacent two or more of R₆₉₁ to R₆₉₄ are
mutually bonded to form a substituted or unsubstituted monocyclic ring, mutually bonded to form a substituted or unsubstituted fused ring, or not mutually bonded;
R₆₀₁ and R₆₀₂ forming neither the substituted or unsubstituted monocyclic ring nor the substituted or unsubstituted fused ring are each independently a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms; and
R₆₀₃ to R₆₀₅, and R₆₁₁ to R₆₁₇ and R₆₉₁ to R₆₉₄ forming neither the substituted or unsubstituted monocyclic ring nor the substituted or unsubstituted fused ring are each independently a hydrogen atom or a substituent Rx, and each substituent Rx is independently a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a halogen atom, a cyano group, a nitro group, a substituted or unsubstituted aryl group having 6 to 50 ring carbon atoms, or a substituted or unsubstituted heterocyclic group having 5 to 50 ring atoms.

The compound represented by the formula (6) is producible by initially bonding the ring a, ring b and ring c with linking groups (a group including N—R₆₀₁ and a group including N—R₆₀₂) to form an intermediate (first reaction), and bonding the ring a, ring b and ring c with a linking group (a group including a boron atom) to form a final product (second reaction). In the first reaction, an amination reaction (e.g. Buchwald-Hartwig reaction) is applicable. In the second reaction, Tandem Hetero-Friedel-Crafts Reactions or the like is applicable.

### Specific Examples of Compound Represented by Formula (6)

Specific examples of the compound represented by the formula (6) are given below. It should however be noted that these specific examples are merely exemplary and do not limit the compound represented by the formula (6).

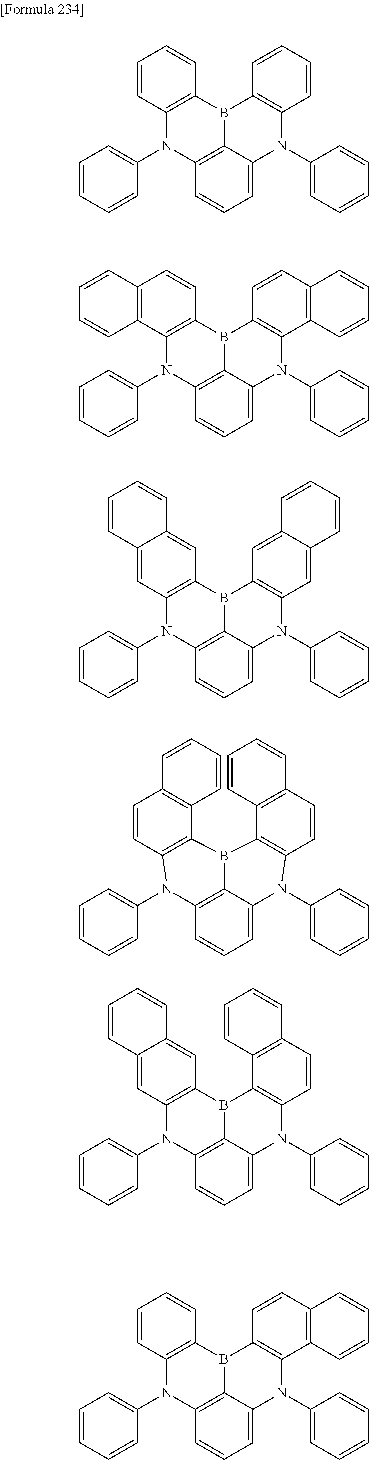

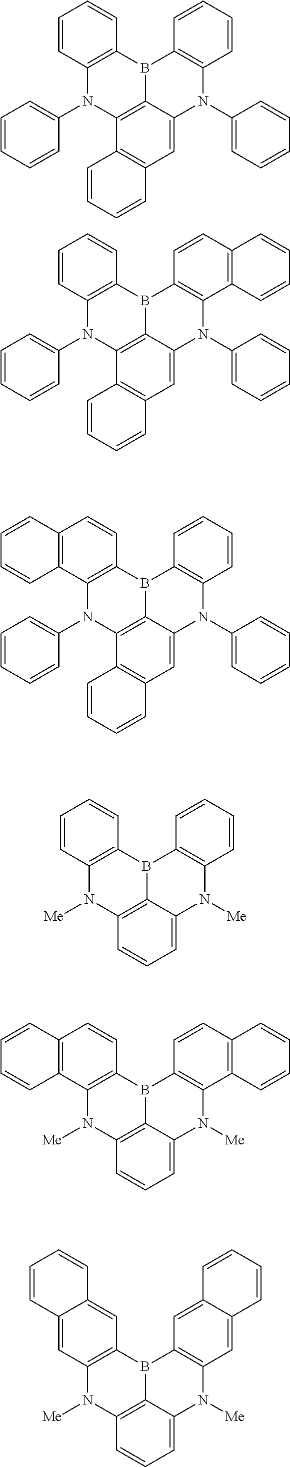

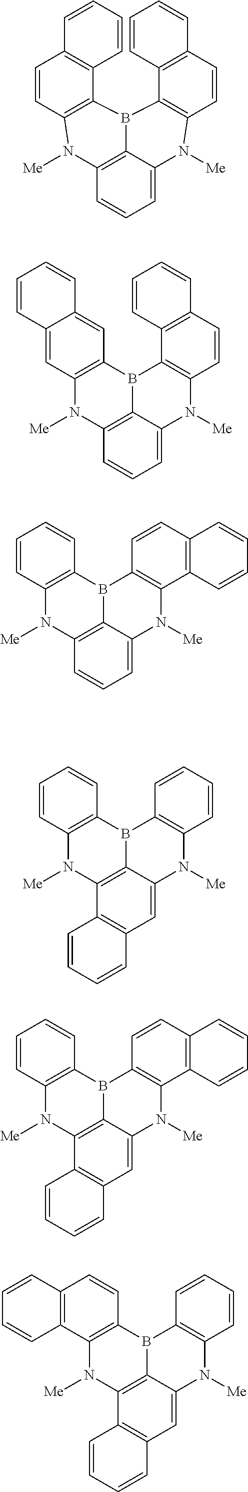

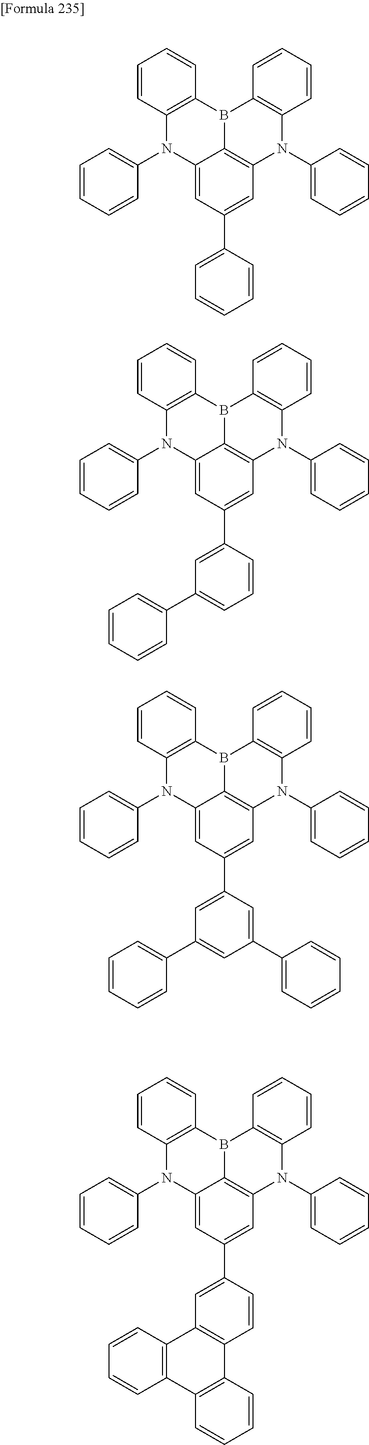

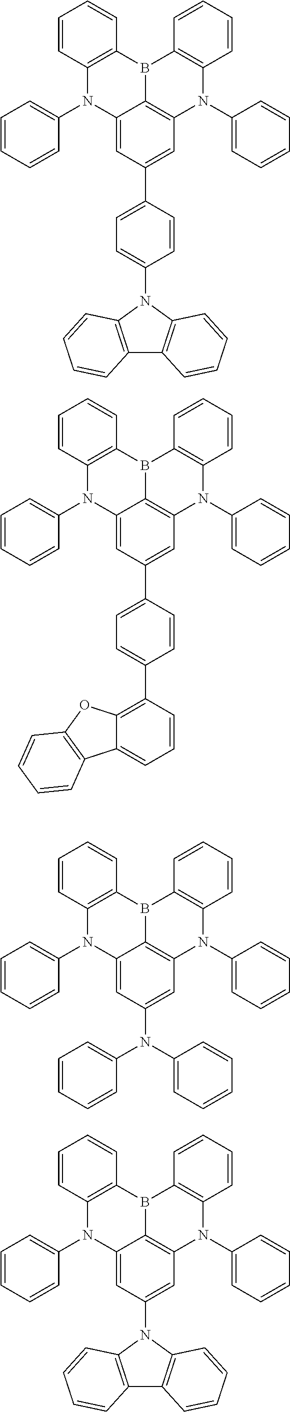

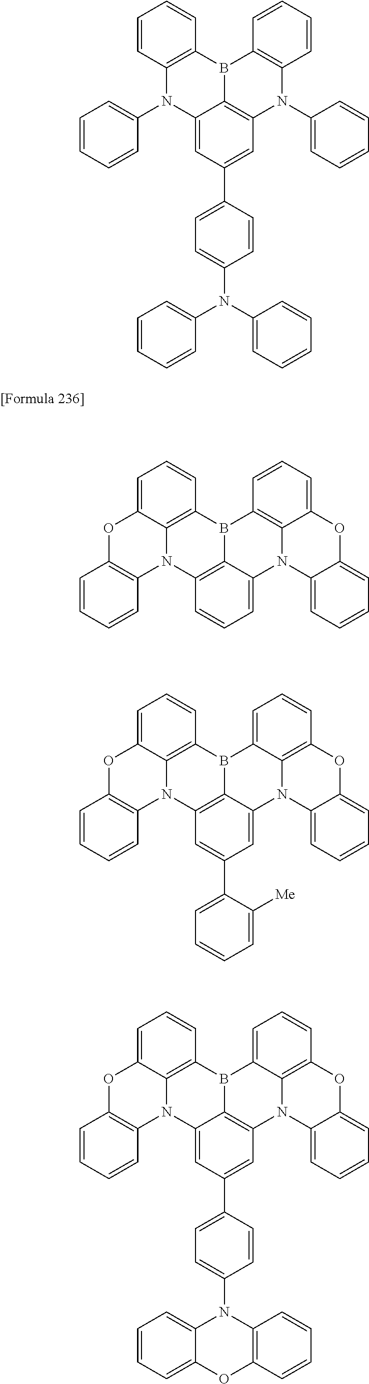 [SMILES: c1ccc(N(c2ccccc2)c2ccc(-c3cc4c5c(c3)N(c3ccccc3)c3ccccc3B5c3ccccc3N4c3ccccc3)cc2)cc1 ; c1ccc2c(c1)Oc1cccc3c1N2c1cccc2c1B3c1cccc3c1N2c1ccccc1O3 ; Cc1ccccc1-c1cc2c3c(c1)N1c4ccccc4Oc4cccc(c41)B3c1cccc3c1N2c1ccccc1O3 ; c1ccc2c(c1)Oc1ccccc1N2c1ccc(-c2cc3c4c(c2)N2c5ccccc5Oc5cccc(c52)B4c2cccc4c2N3c2ccccc2O4)cc1]

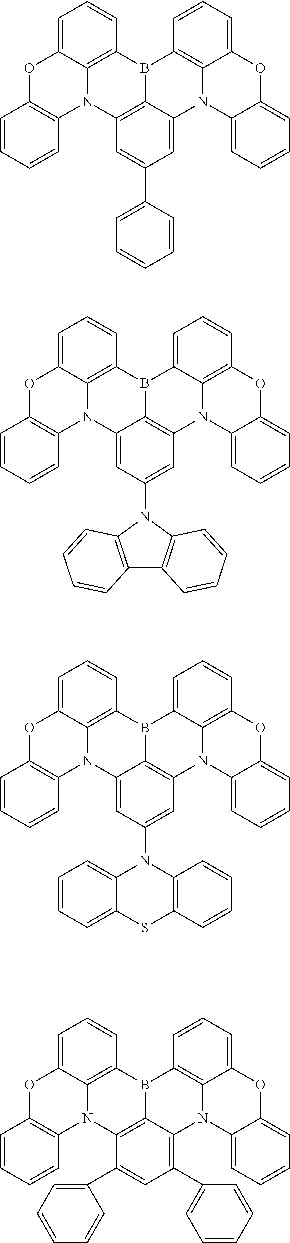 [SMILES: c1ccc(-c2cc3c4c(c2)N2c5ccccc5Oc5cccc(c52)B4c2cccc4c2N3c2ccccc2O4)cc1 ; c1ccc2c(c1)Oc1cccc3c1N2c1cc(-n2c4ccccc4c4ccccc42)cc2c1B3c1cccc3c1N2c1ccccc1O3 ; c1ccc2c(c1)Oc1cccc3c1N2c1cc(N2c4ccccc4Sc4ccccc42)cc2c1B3c1cccc3c1N2c1ccccc1O3 ; c1ccc(-c2cc(-c3ccccc3)c3c4c2N2c5ccccc5Oc5cccc(c52)B4c2cccc4c2N3c2ccccc2O4)cc1]

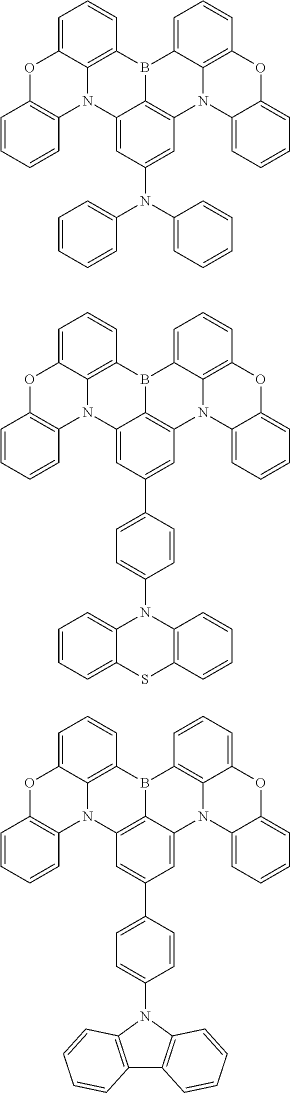

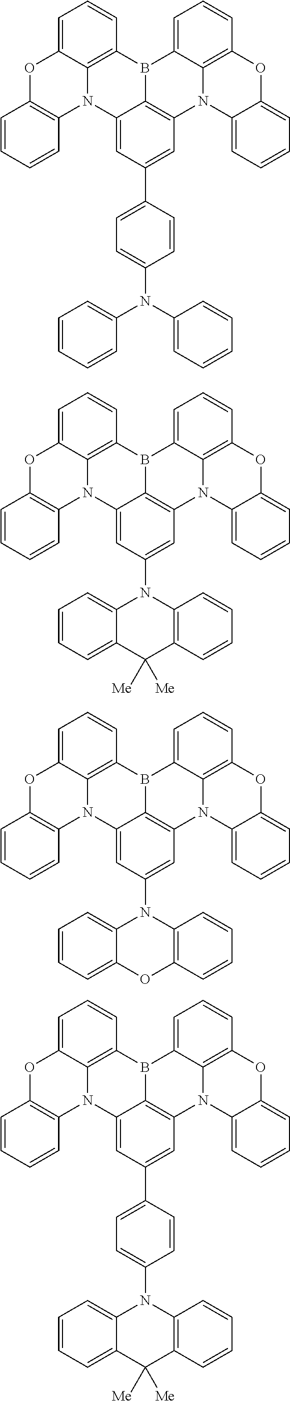

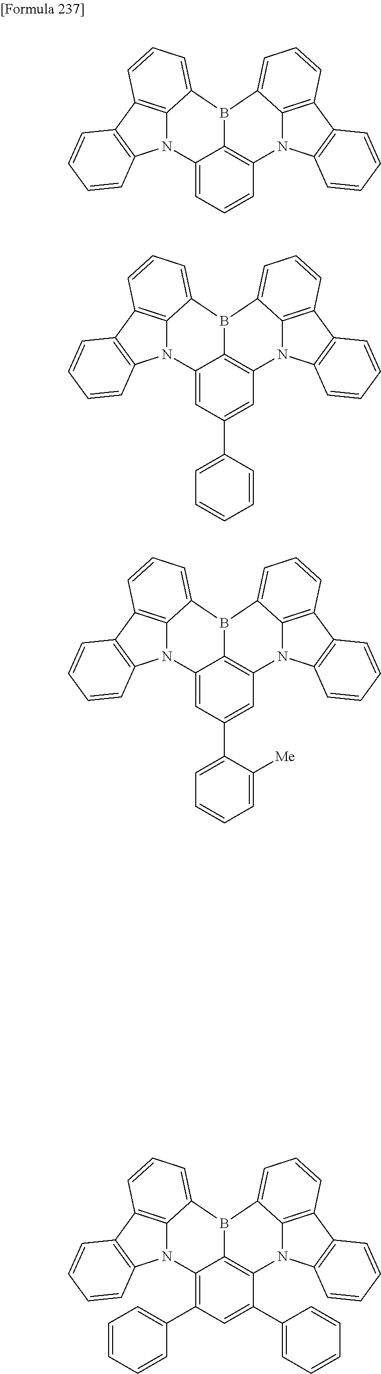 [SMILES: c1cc2c3c(c1)-n1c4ccccc4c4cccc(c41)B3c1cccc3c4ccccc4n-2c13 ; c1ccc(-c2cc3c4c(c2)-n2c5ccccc5c5cccc(c52)B4c2cccc4c5ccccc5n-3c24)cc1 ; Cc1ccccc1-c1cc2c3c(c1)-n1c4ccccc4c4cccc(c41)B3c1cccc3c4ccccc4n-2c13 ; c1ccc(-c2cc(-c3ccccc3)c3c4c2-n2c5ccccc5c5cccc(c52)B4c2cccc4c5ccccc5n-3c24)cc1]

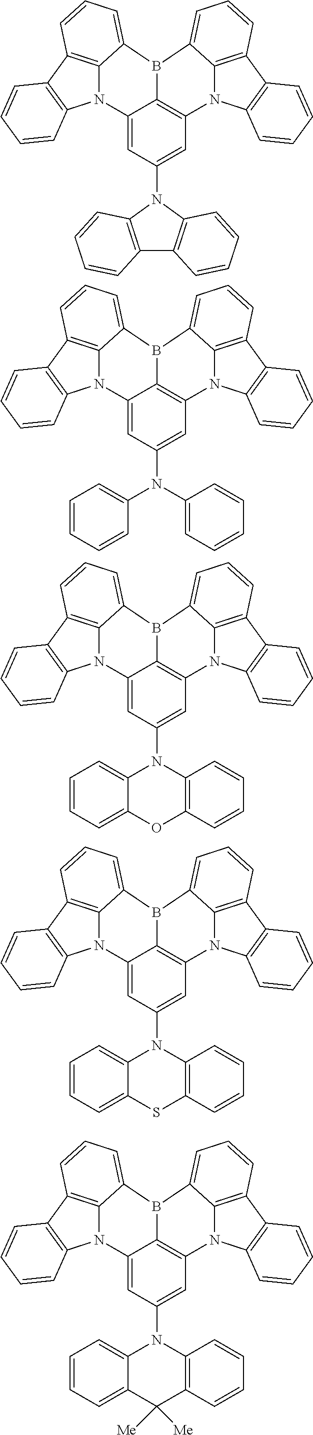

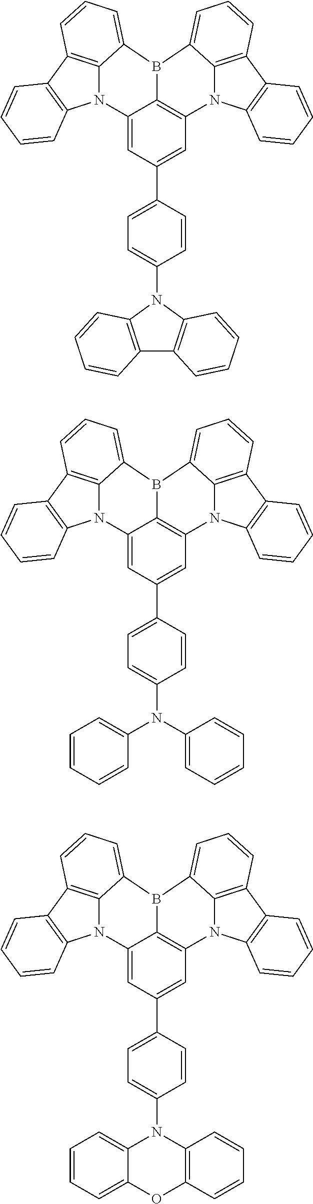 [SMILES: c1ccc2c(c1)c1ccccc1n2-c1ccc(-c2cc3c4c(c2)-n2c5ccccc5c5cccc(c52)B4c2cccc4c5ccccc5n-3c24)cc1 ; c1ccc(N(c2ccccc2)c2ccc(-c3cc4c5c(c3)-n3c6ccccc6c6cccc(c63)B5c3cccc5c6ccccc6n-4c35)cc2)cc1 ; c1ccc2c(c1)Oc1ccccc1N2c1ccc(-c2cc3c4c(c2)-n2c5ccccc5c5cccc(c52)B4c2cccc4c5ccccc5n-3c24)cc1]

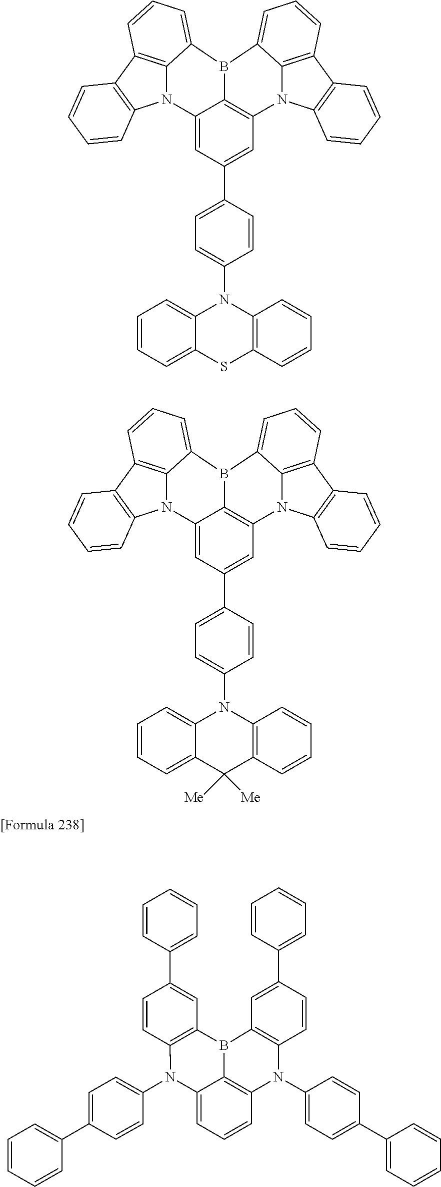

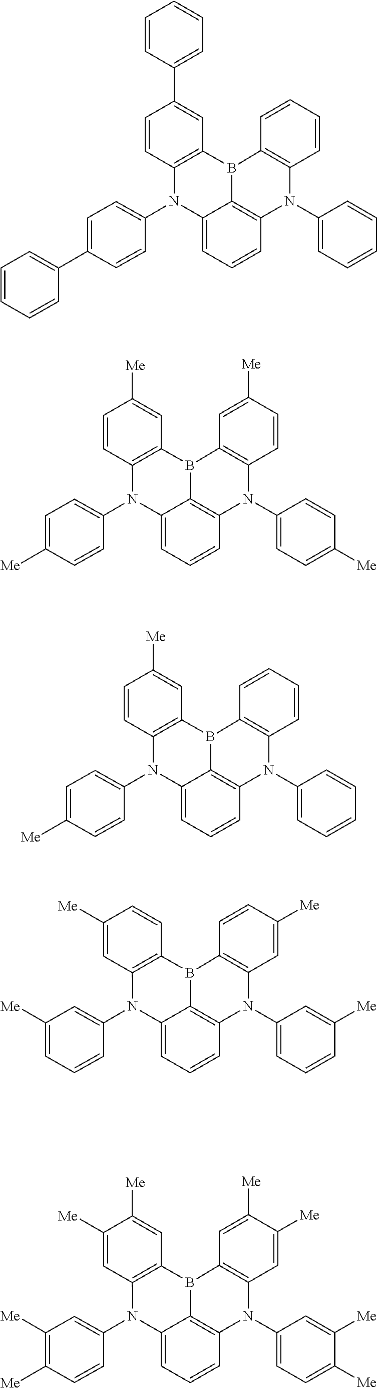 [SMILES: c1ccc(-c2ccc(N3c4ccc(-c5ccccc5)cc4B4c5ccccc5N(c5ccccc5)c5cccc3c54)cc2)cc1 ; Cc1ccc(N2c3ccc(C)cc3B3c4cc(C)ccc4N(c4ccc(C)cc4)c4cccc2c43)cc1 ; Cc1ccc(N2c3ccc(C)cc3B3c4ccccc4N(c4ccccc4)c4cccc2c43)cc1 ; Cc1cccc(N2c3cc(C)ccc3B3c4ccc(C)cc4N(c4cccc(C)c4)c4cccc2c43)c1 ; Cc1ccc(N2c3cc(C)c(C)cc3B3c4cc(C)c(C)cc4N(c4ccc(C)c(C)c4)c4cccc2c43)cc1C]

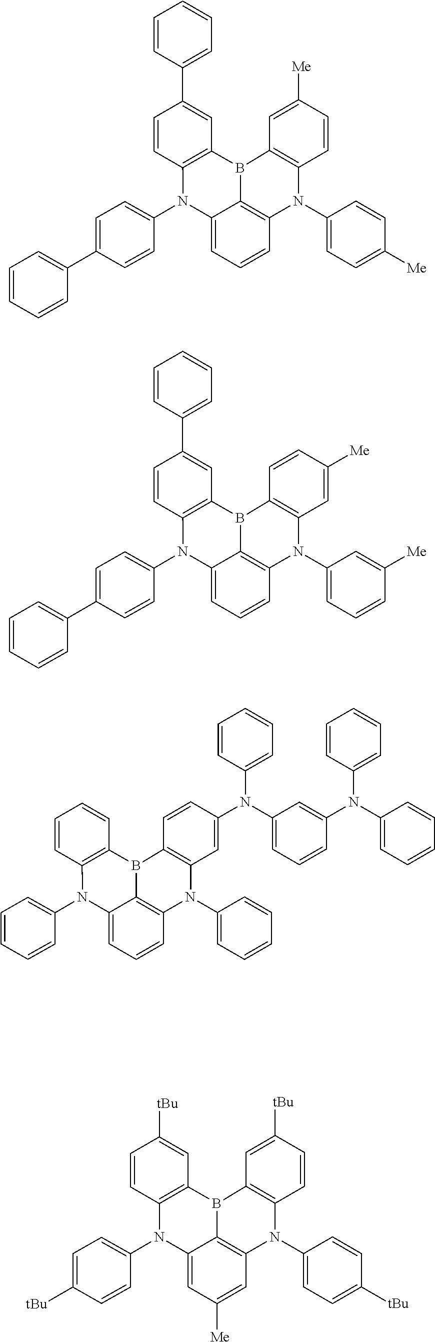 [SMILES: Cc1ccc(N2c3ccc(C)cc3B3c4cc(-c5ccccc5)ccc4N(c4ccc(-c5ccccc5)cc4)c4cccc2c43)cc1 ; Cc1cccc(N2c3cc(C)ccc3B3c4cc(-c5ccccc5)ccc4N(c4ccc(-c5ccccc5)cc4)c4cccc2c43)c1 ; c1ccc(N(c2ccccc2)c2cccc(N(c3ccccc3)c3ccc4c(c3)N(c3ccccc3)c3cccc5c3B4c3ccccc3N5c3ccccc3)c2)cc1 ; Cc1cc2c3c(c1)N(c1ccc(C(C)(C)C)cc1)c1ccc(C(C)(C)C)cc1B3c1cc(C(C)(C)C)ccc1N2c1ccc(C(C)(C)C)cc1]

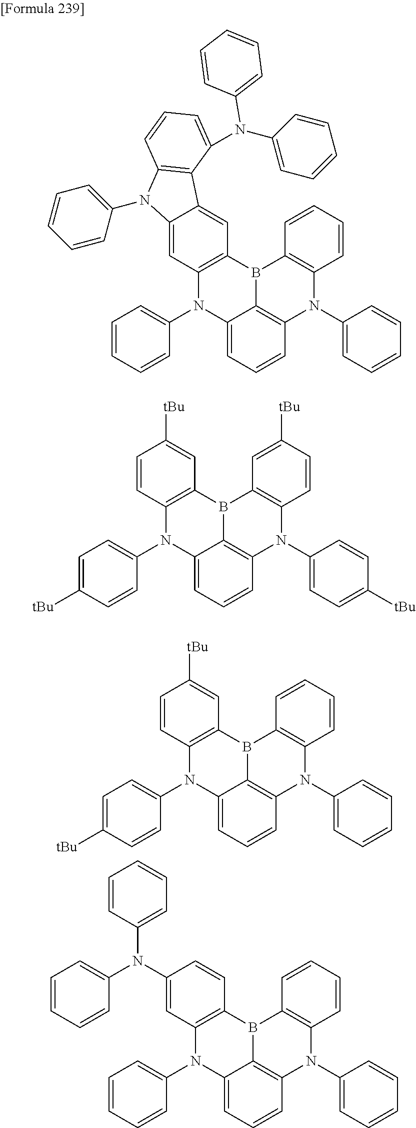

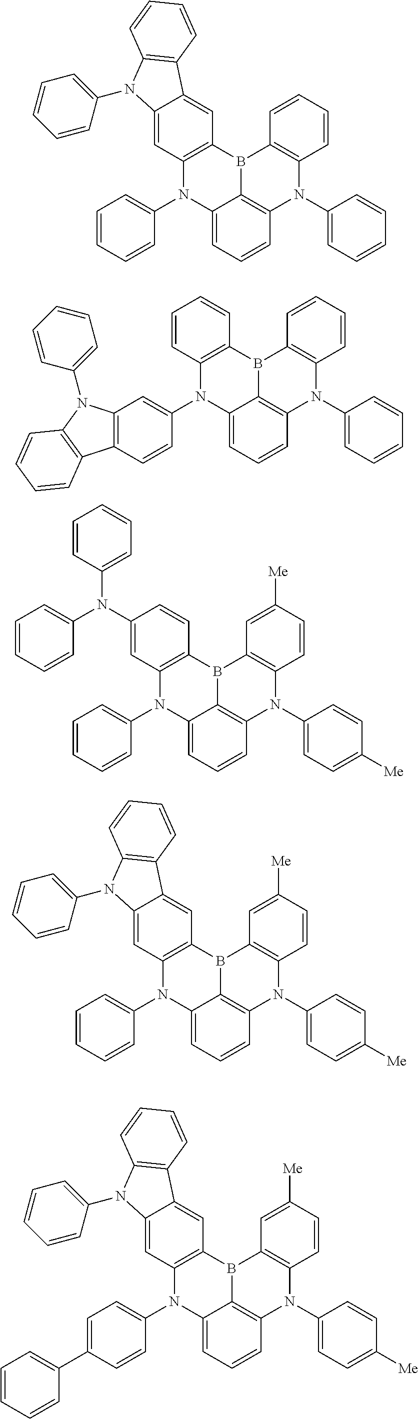

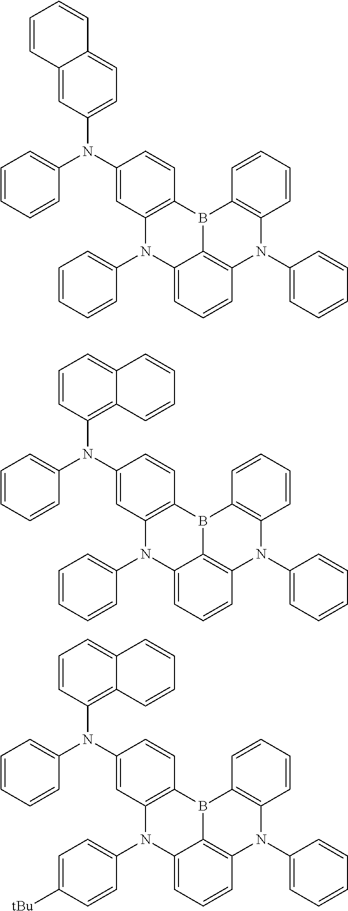

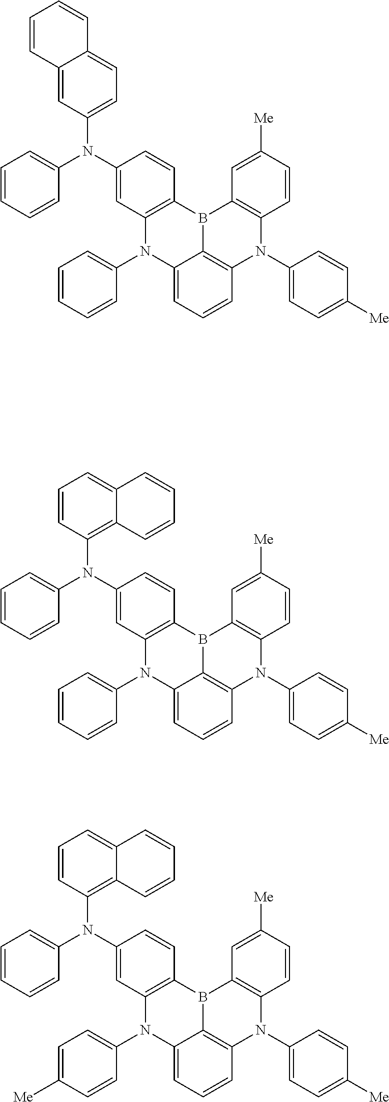 [SMILES: Cc1ccc(N2c3ccc(C)cc3B3c4ccc(N(c5ccccc5)c5ccc6ccccc6c5)cc4N(c4ccccc4)c4cccc2c43)cc1 ; Cc1ccc(N2c3ccc(C)cc3B3c4ccc(N(c5ccccc5)c5cccc6ccccc56)cc4N(c4ccccc4)c4cccc2c43)cc1 ; Cc1ccc(N2c3ccc(C)cc3B3c4ccc(N(c5ccccc5)c5cccc6ccccc56)cc4N(c4ccc(C)cc4)c4cccc2c43)cc1]

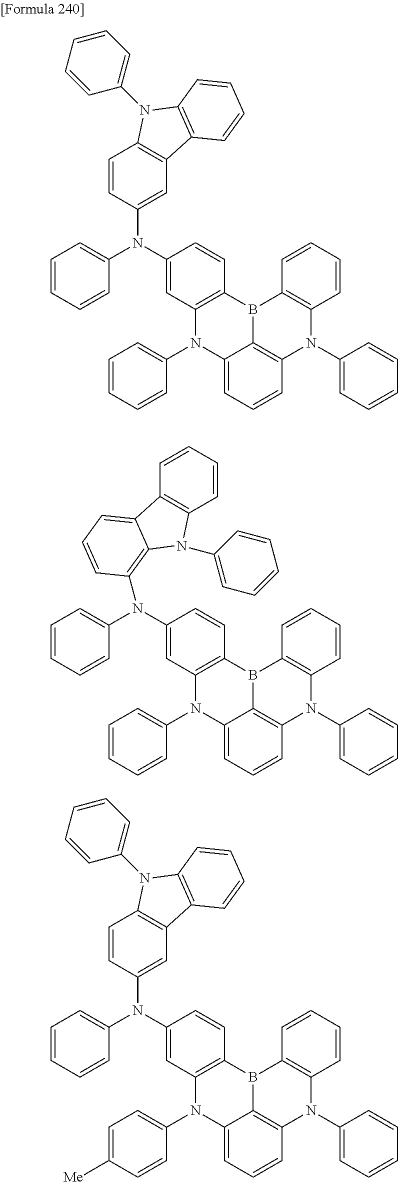

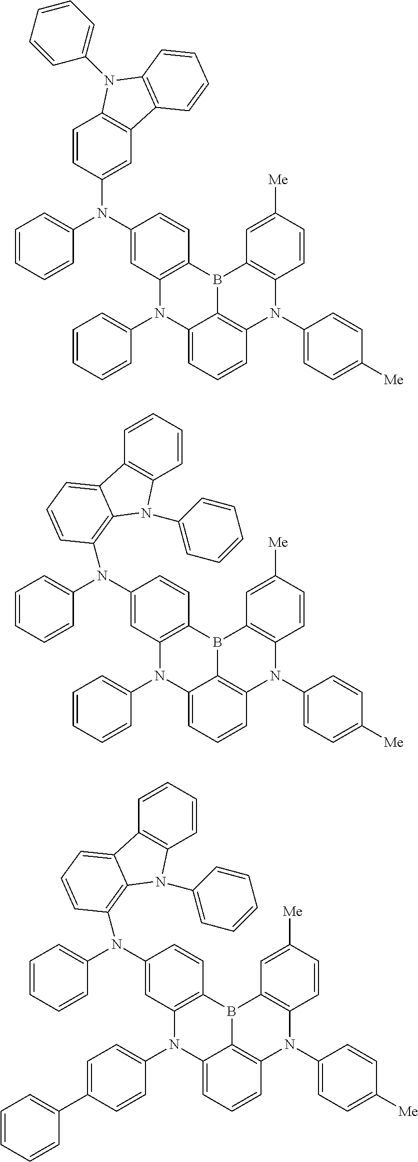 [SMILES: Cc1ccc(N2c3ccc(C)cc3B3c4ccc(N(c5ccccc5)c5ccc6c(c5)c5ccccc5n6-c5ccccc5)cc4N(c4ccccc4)c4cccc2c43)cc1 ; Cc1ccc(N2c3ccc(C)cc3B3c4ccc(N(c5ccccc5)c5cccc6c7ccccc7n(-c7ccccc7)c56)cc4N(c4ccccc4)c4cccc2c43)cc1 ; Cc1ccc(N2c3ccc(C)cc3B3c4ccc(N(c5ccccc5)c5cccc6c7ccccc7n(-c7ccccc7)c56)cc4N(c4ccc(-c5ccccc5)cc4)c4cccc2c43)cc1]

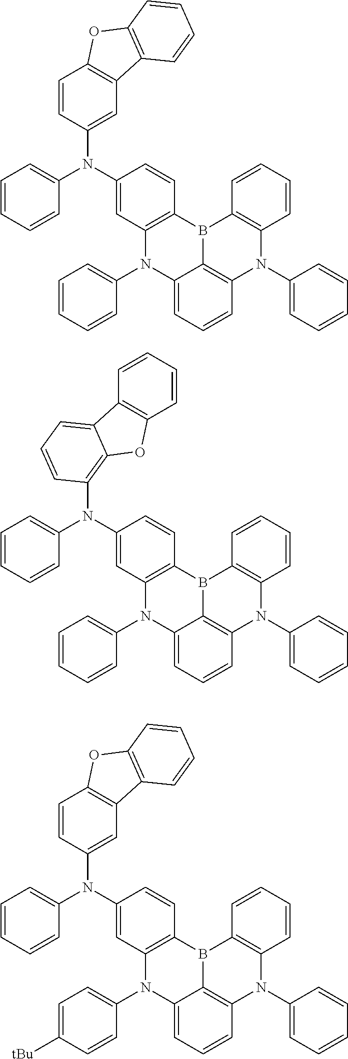 [SMILES: c1ccc(N(c2ccc3c(c2)N(c2ccccc2)c2cccc4c2B3c2ccccc2N4c2ccccc2)c2ccc3oc4ccccc4c3c2)cc1 ; c1ccc(N2c3ccccc3B3c4ccc(N(c5ccccc5)c5cccc6c5oc5ccccc56)cc4N(c4ccccc4)c4cccc2c43)cc1 ; CC(C)(C)c1ccc(N2c3cc(N(c4ccccc4)c4ccc5oc6ccccc6c5c4)ccc3B3c4ccccc4N(c4ccccc4)c4cccc2c43)cc1]

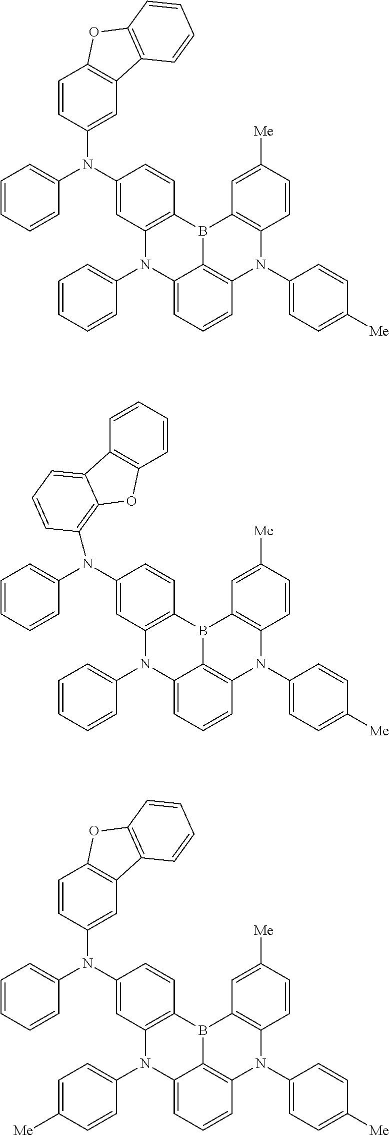 [SMILES: Cc1ccc(N2c3ccc(C)cc3B3c4ccc(N(c5ccccc5)c5ccc6oc7ccccc7c6c5)cc4N(c4ccccc4)c4cccc2c43)cc1 ; Cc1ccc(N2c3ccc(C)cc3B3c4ccc(N(c5ccccc5)c5cccc6c5oc5ccccc56)cc4N(c4ccccc4)c4cccc2c43)cc1 ; Cc1ccc(N2c3ccc(C)cc3B3c4ccc(N(c5ccccc5)c5ccc6oc7ccccc7c6c5)cc4N(c4ccc(C)cc4)c4cccc2c43)cc1]

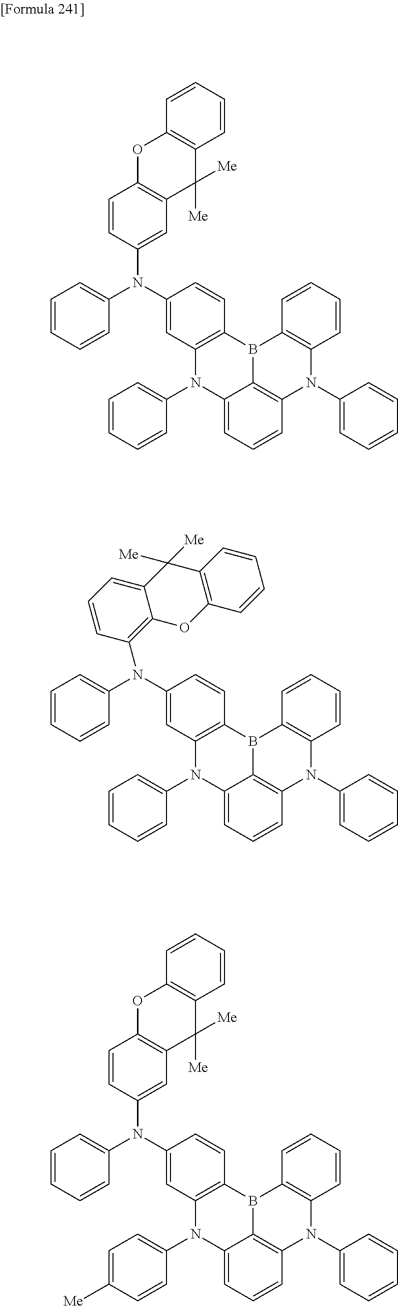 [SMILES: CC1(C)c2ccccc2Oc2ccc(N(c3ccccc3)c3ccc4c(c3)N(c3ccccc3)c3cccc5c3B4c3ccccc3N5c3ccccc3)cc21 ; CC1(C)c2ccccc2Oc2c(N(c3ccccc3)c3ccc4c(c3)N(c3ccccc3)c3cccc5c3B4c3ccccc3N5c3ccccc3)cccc21 ; Cc1ccc(N2c3cc(N(c4ccccc4)c4ccc5c(c4)C(C)(C)c4ccccc4O5)ccc3B3c4ccccc4N(c4ccccc4)c4cccc2c43)cc1]

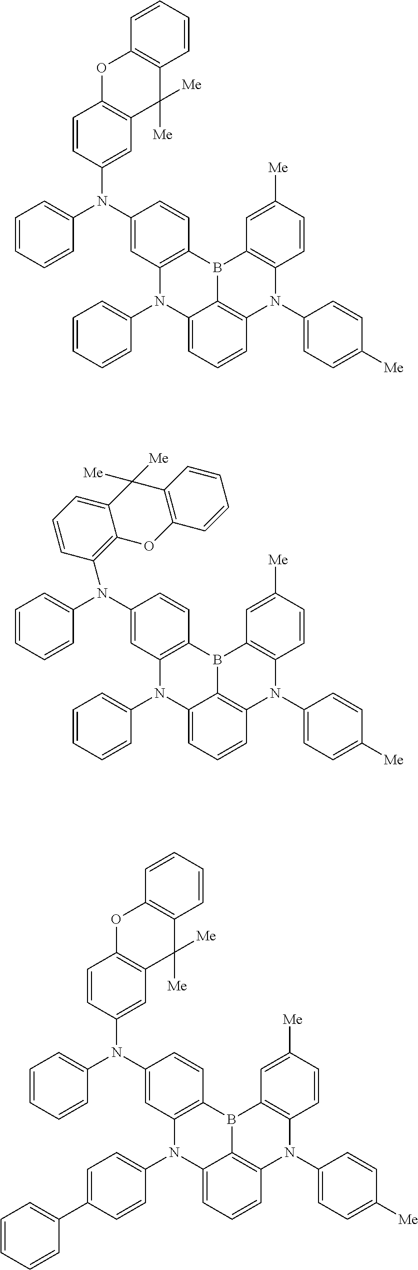 [SMILES: Cc1ccc(N2c3ccc(C)cc3B3c4ccc(N(c5ccccc5)c5ccc6c(c5)C(C)(C)c5ccccc5O6)cc4N(c4ccccc4)c4cccc2c43)cc1 ; Cc1ccc(N2c3ccc(C)cc3B3c4ccc(N(c5ccccc5)c5cccc6c5Oc5ccccc5C6(C)C)cc4N(c4ccccc4)c4cccc2c43)cc1 ; Cc1ccc(N2c3ccc(C)cc3B3c4ccc(N(c5ccccc5)c5ccc6c(c5)C(C)(C)c5ccccc5O6)cc4N(c4ccc(-c5ccccc5)cc4)c4cccc2c43)cc1]

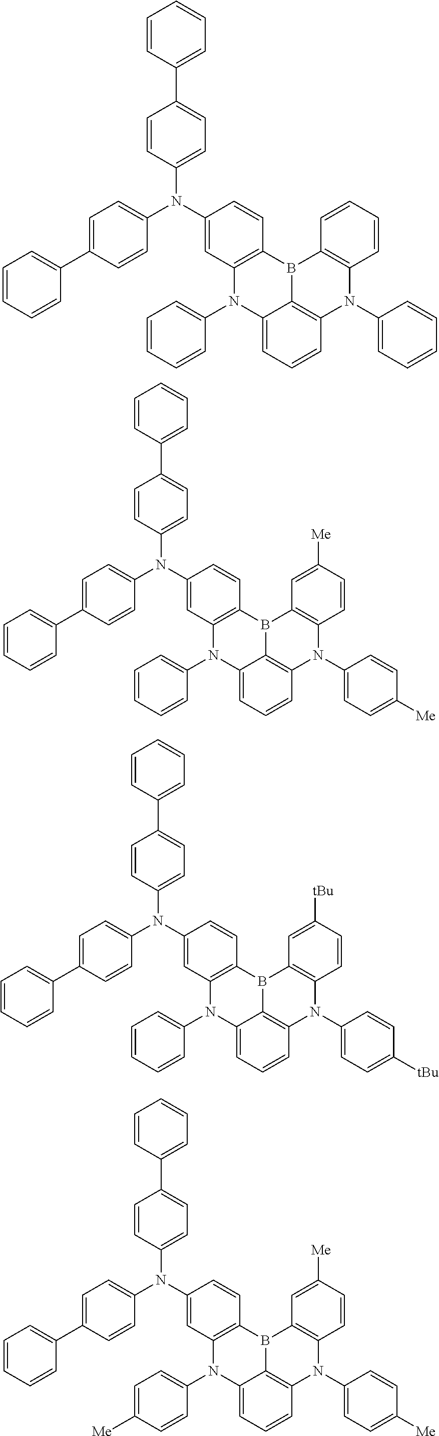

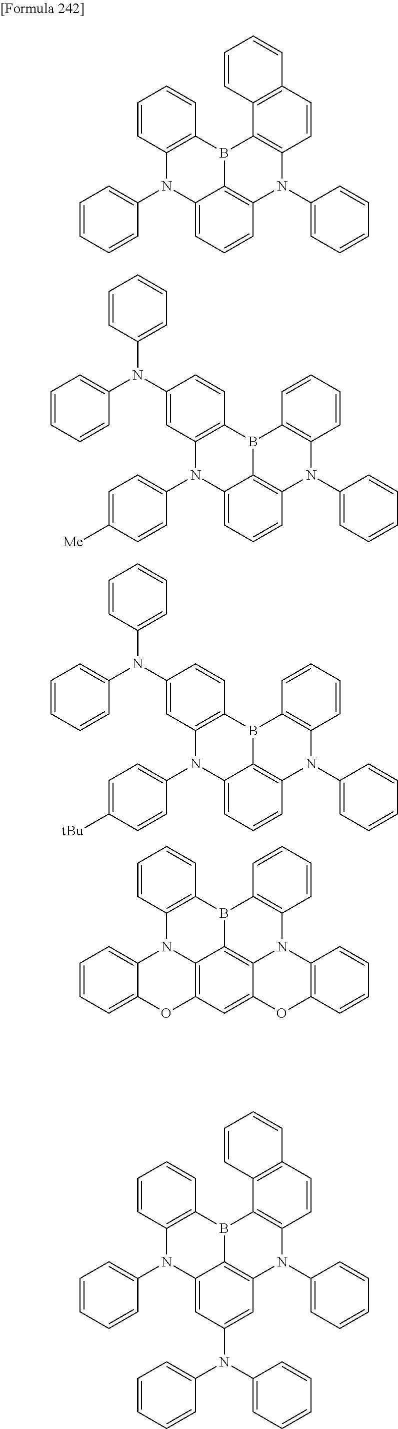

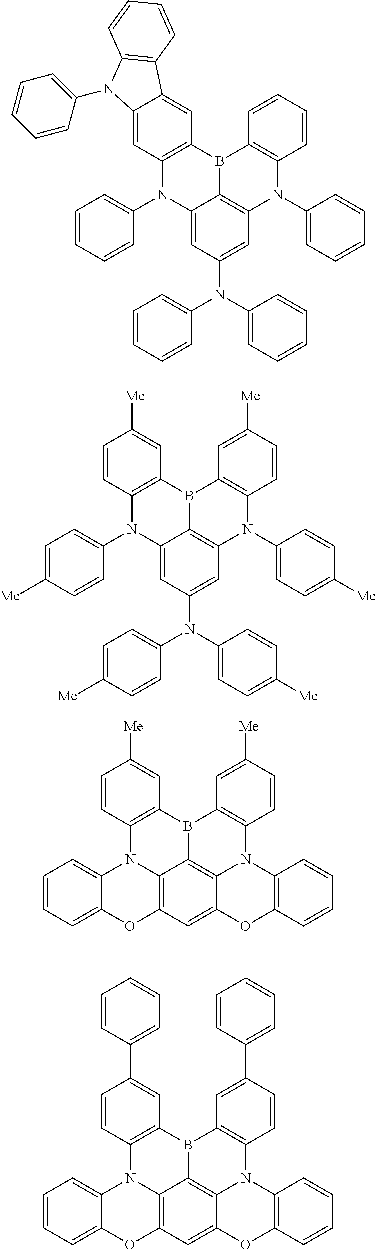

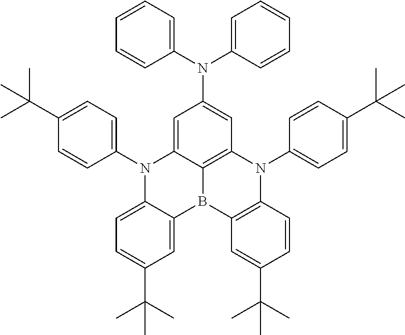 [SMILES: CC(C)(C)c1ccc(N2c3ccc(C(C)(C)C)cc3B3c4cc(C(C)(C)C)ccc4N(c4ccc(C(C)(C)C)cc4)c4cc(N(c5ccccc5)c5ccccc5)cc2c43)cc1]

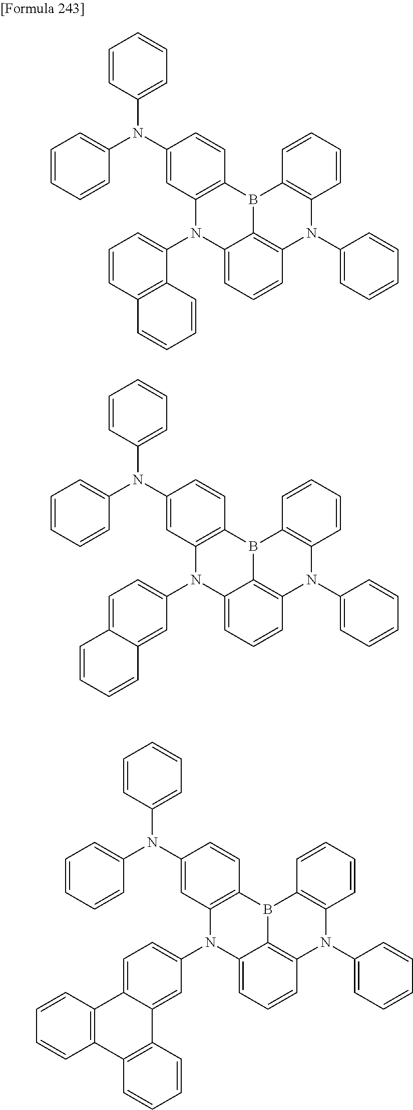

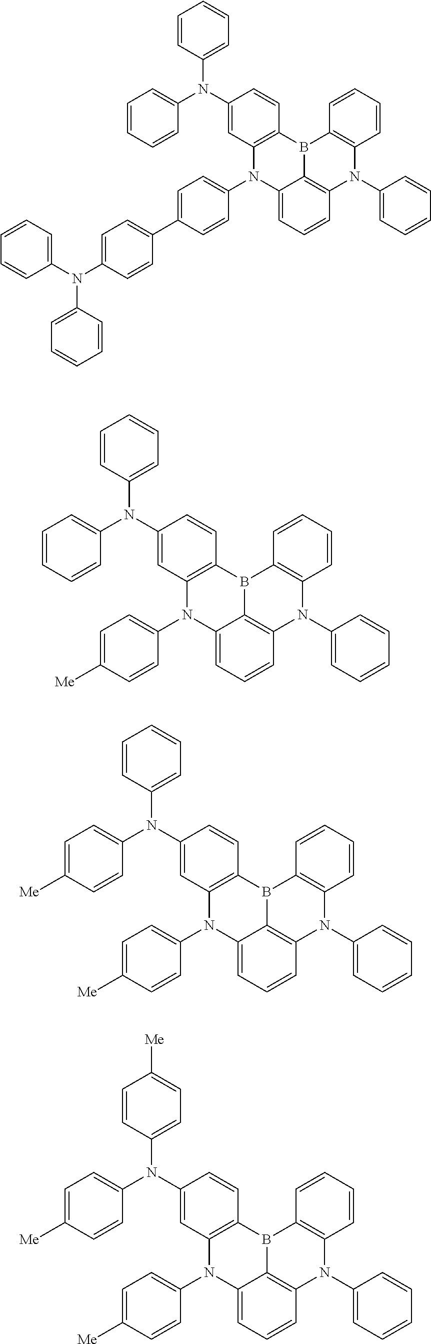 [SMILES: c1ccc(N(c2ccccc2)c2ccc(-c3ccc(N4c5cc(N(c6ccccc6)c6ccccc6)ccc5B5c6ccccc6N(c6ccccc6)c6cccc4c65)cc3)cc2)cc1 ; Cc1ccc(N2c3cc(N(c4ccccc4)c4ccccc4)ccc3B3c4ccccc4N(c4ccccc4)c4cccc2c43)cc1 ; Cc1ccc(N(c2ccccc2)c2ccc3c(c2)N(c2ccc(C)cc2)c2cccc4c2B3c2ccccc2N4c2ccccc2)cc1 ; Cc1ccc(N(c2ccc(C)cc2)c2ccc3c(c2)N(c2ccc(C)cc2)c2cccc4c2B3c2ccccc2N4c2ccccc2)cc1]

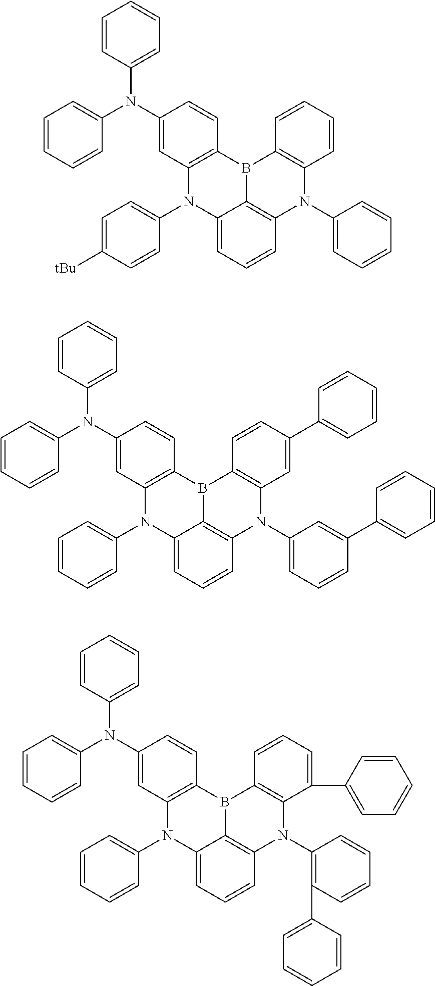 [SMILES: CC(C)(C)c1ccc(N2c3cc(N(c4ccccc4)c4ccccc4)ccc3B3c4ccccc4N(c4ccccc4)c4cccc2c43)cc1 ; c1ccc(-c2cccc(N3c4cc(-c5ccccc5)ccc4B4c5ccc(N(c6ccccc6)c6ccccc6)cc5N(c5ccccc5)c5cccc3c54)c2)cc1 ; c1ccc(-c2ccccc2N2c3cccc4c3B(c3ccc(N(c5ccccc5)c5ccccc5)cc3N4c3ccccc3)c3cccc(-c4ccccc4)c32)cc1]

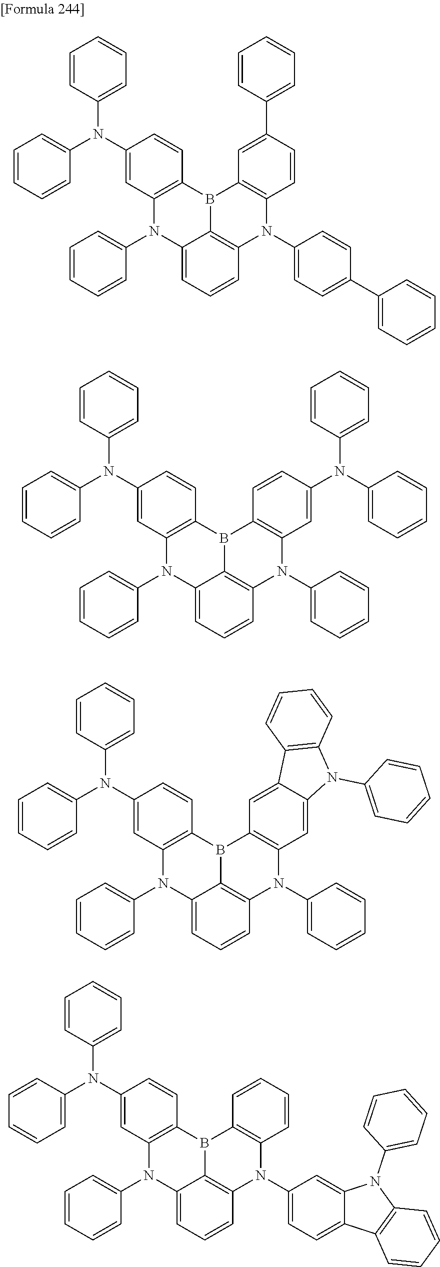

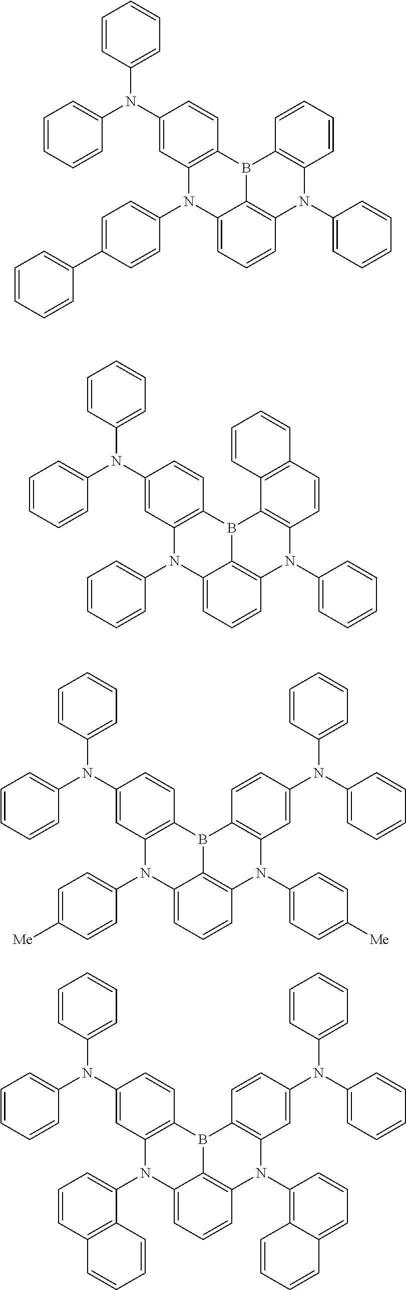

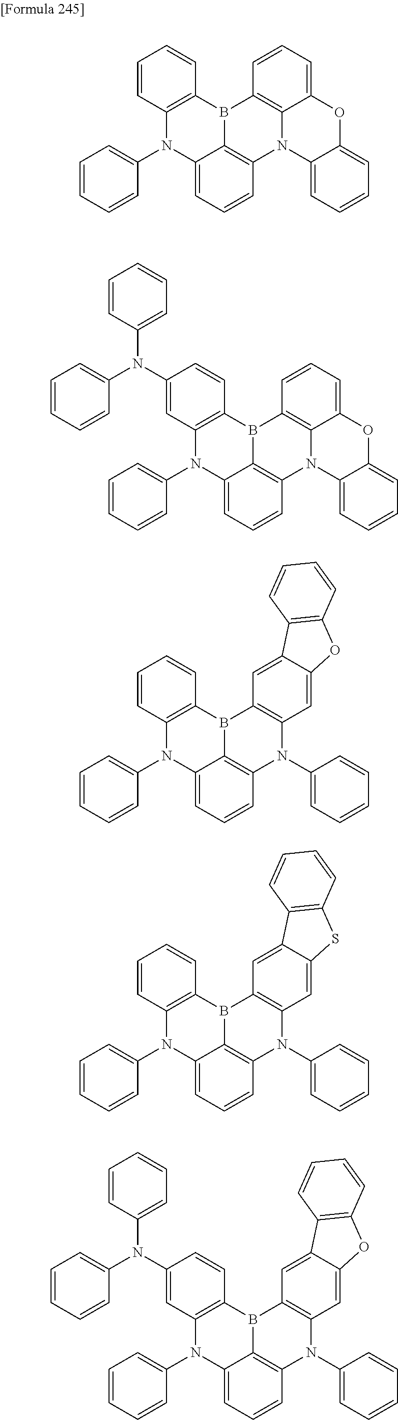

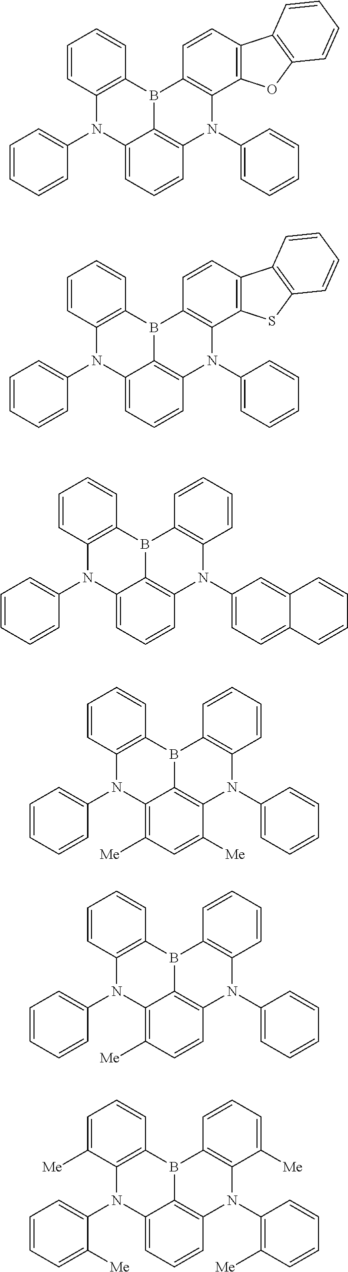

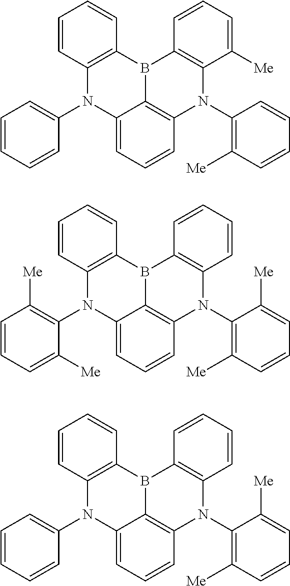

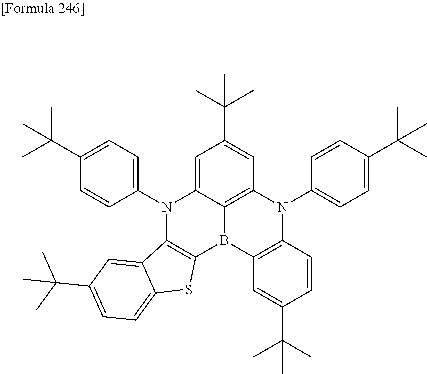 [SMILES: CC(C)(C)c1ccc(N2c3ccc(C(C)(C)C)cc3B3c4sc5ccc(C(C)(C)C)cc5c4N(c4ccc(C(C)(C)C)cc4)c4cc(C(C)(C)C)cc2c43)cc1]

In an exemplary arrangement of the organic EL device of each of the exemplary embodiments, a triplet energy of the first luminescent compound T₁(D1) satisfies a relationship of a numerical formula (Numerical Formula 15) below.

$$2.6 eV > {T}_{1} ( D 1 ) > {T}_{1} ( H 1 ) > {T}_{1} ( H 3 ) \qquad ( Numerical Formula 15 )$$

In an exemplary arrangement of the organic EL device of each of the exemplary embodiments, the triplet energy of the first luminescent compound T₁(D1) satisfies a relationship of a numerical formula (Numerical Formula 16) below.

$$2.6 eV > {T}_{1} ( D 1 ) > {T}_{1} ( H 2 ) > {T}_{1} ( H 3 ) \qquad ( Numerical Formula 16 )$$

In an exemplary arrangement of the organic EL device of each of the exemplary embodiments, the triplet energy of the second luminescent compound T₁(D2) satisfies a relationship of a numerical formula (Numerical Formula 17) below.

$$2.6 eV > {T}_{1} ( D 2 ) > {T}_{1} ( H 1 ) > {T}_{1} ( H 3 ) \qquad ( Numerical Formula 17 )$$

In an exemplary arrangement of the organic EL device of each of the exemplary embodiments, the triplet energy of the second luminescent compound T₁(D2) satisfies a relationship of a numerical formula (Numerical Formula 18) below.

$$2.6 eV > {T}_{1} ( D 2 ) > {T}_{1} ( H 2 ) > {T}_{1} ( H 3 ) \qquad ( Numerical Formula 18 )$$

In an exemplary arrangement of the organic EL device of each of the exemplary embodiments, the triplet energy of the first luminescent compound T₁ (D1) satisfies a relationship of a numerical formula (Numerical Formula 19) below.

$$0 eV < {T}_{1} ( D 1 ) - {T}_{1} ( H 1 ) < 0.6 eV \qquad ( Numerical Formula 19 )$$

In an exemplary arrangement of the organic EL device of each of the exemplary embodiments, the triplet energy of the first luminescent compound T₁(D1) satisfies a relationship of a numerical formula (Numerical Formula 25) below.

$$0 eV < {T}_{1} ( D 1 ) - {T}_{1} ( H 2 ) < 0.6 eV \qquad ( Numerical Formula 25 )$$

In an exemplary arrangement of the organic EL device of each of the exemplary embodiments, the triplet energy of the second luminescent compound T₁ (D2) satisfies a relationship of a numerical formula (Numerical Formula 26) below.

$$0 eV < {T}_{1} ( D 2 ) - {T}_{1} ( H 3 ) < 0.8 eV \qquad ( Numerical Formula 26 )$$

In an exemplary arrangement of the organic EL device of each of the exemplary embodiments, the triplet energy of the first host material T₁(H1) satisfies a relationship of a numerical formula (Numerical Formula 27) below.

$${T}_{1} ( H 1 ) > 2. eV \qquad ( Numerical Formula 27 )$$

In an exemplary arrangement of the organic EL device of each of the exemplary embodiments, the triplet energy of the first host material T₁(H1) satisfies a relationship of a numerical formula (Numerical Formula 27A) or a numerical formula (Numerical Formula 27B) below.

$${T}_{1} ( H 1 ) > 2.1 eV \qquad ( Numerical Formula 27 A )$$

$${T}_{1} ( H 1 ) > 2.15 eV \qquad ( Numerical Formula 27 B )$$

In an exemplary arrangement of the organic EL device of each of the exemplary embodiments, when the triplet energy of the first host material T₁(H1) satisfies the relationship of the numerical formula (Numerical Formula 27A) or the numerical formula (Numerical Formula 27B), triplet excitons generated in the first emitting layer easily transfer to the second emitting layer, and also are easily inhibited from back-transferring from the second emitting layer to the first emitting layer. Consequently, singlet excitons are efficiently generated in the second emitting layer, thereby improving luminous efficiency.

In an exemplary arrangement of the organic EL device of each of the exemplary embodiments, the triplet energy of the first host material T₁(H1) satisfies a relationship of a numerical formula (Numerical Formula 27C) or a numerical formula (Numerical Formula 27D) below.

$$2.08 eV > {T}_{1} ( H 1 ) > 1.87 eV \qquad ( Numerical Formula 27 C )$$

$$2.05 eV > {T}_{1} ( H 1 ) > 1.9 eV \qquad ( Numerical Formula 27 D )$$

When the triplet energy of the first host material T₁(H1) satisfies the relationship of the numerical formula (Numerical Formula 27C) or the numerical formula (Numerical Formula 27D), the energy of triplet excitons generated in the first emitting layer is reduced. The organic EL device can thus be expected to have a long lifetime.

In an exemplary arrangement of the organic EL device of each of the exemplary embodiments, the triplet energy of the first luminescent compound T₁(D1) satisfies a relationship of a numerical formula (Numerical Formula 28A) or a numerical formula (Numerical Formula 28B) below.

$$2.6 eV > {T}_{1} ( D 1 ) \qquad ( Numerical Formula 28 A )$$

$$2.5 eV > {T}_{1} ( D 1 ) \qquad ( Numerical Formula 28 B )$$

The organic EL device has a long lifetime when the first emitting layer contains a compound that satisfies the relationship of the numerical formula (Numerical Formula 28A) or the numerical formula (Numerical Formula 28B).

In an exemplary arrangement of the organic EL device of each of the exemplary embodiments, the triplet energy of the second luminescent compound T₁(D2) satisfies a relationship of a numerical formula (Numerical Formula 28C) or a numerical formula (Numerical Formula 28D) below.

$$2.6 eV > {T}_{1} ( D 2 ) \qquad ( Numerical Formula 28 C )$$

$$2.5 eV > {T}_{1} ( D 2 ) \qquad ( Numerical Formula 28 D )$$

The organic EL device has a long lifetime when the second emitting layer contains a compound that satisfies the relationship of the numerical formula (Numerical Formula 28C) or the numerical formula (Numerical Formula 28D).

In an exemplary arrangement of the organic EL device of each of the exemplary embodiments, the first emitting layer and the second emitting layer are in direct contact with each other.

Herein, a layer arrangement in which “the first emitting layer and the second emitting layer are in direct contact with each other” may include one of embodiments (LS1), (LS2), and (LS3) below.

(LS1) An embodiment in which a region containing the first host material, the second host material, and the third host material is generated in a process of vapor-depositing the compound of the first emitting layer and vapor-depositing the compound of the second emitting layer, and is present on the interface between the first emitting layer and the second emitting layer.

(LS2) An embodiment in which in a case of containing a luminescent compound in the first emitting layer and the second emitting layer, a region containing the first host material, the second host material, the third host material, and the luminescent compound is generated in a process of vapor-depositing the compound of the first emitting layer and vapor-depositing the compound of the second emitting layer, and is present on the interface between the first emitting layer and the second emitting layer.

(LS3) An embodiment in which in a case of containing a luminescent compound in the first emitting layer and the second emitting layer, a region containing the luminescent compound, a region containing the first host material, or a region containing the second host material and the third host material is generated in a process of vapor-depositing the compound of the first emitting layer and vapor-depositing the compound of the second emitting layer, and is present on the interface between the first emitting layer and the second emitting layer.

In an exemplary arrangement of the organic EL device of each of the exemplary embodiments, the first emitting layer and the second emitting layer are not in direct contact with each other, and an organic layer is provided between the first emitting layer and the second emitting layer.

### Interposed Layer

The organic EL device of each of the exemplary embodiments may include, as the organic layer disposed between the first emitting layer and the second emitting layer, an interposed layer. That is, the first emitting layer, the second emitting layer, and the interposed layer may be provided in the emitting region of the organic EL device of each of the exemplary embodiments.

In the organic EL device of each of the exemplary embodiments, in order to inhibit an overlap between a Singlet emitting region and a TTF emitting region, the interposed layer contains no luminescent compound or may contain a luminescent compound in an insubstantial amount provided that the overlap can be inhibited.

For instance, the interposed layer contains 0 mass % of a luminescent compound. Alternatively, for instance, the interposed layer may contain a luminescent compound provided that the luminescent compound contained is a component accidentally mixed in a producing process or a component contained as impurities in a material.

For instance, when the interposed layer consists of a material A, a material B, and a material C, the content ratios of the materials A, B, and C in the interposed layer are each 10 mass % or more, and the total of the content ratios of the materials A, B, and C is 100 mass %.

In the following, the interposed layer is occasionally referred to as a “non-doped layer”. A layer containing a luminescent compound is occasionally referred to as a “doped layer”.

It is considered that luminous efficiency is improvable in an arrangement including layered emitting layers because the Singlet emitting region and the TTF emitting region are typically likely to be separated from each other.

In the organic EL device of each of the exemplary embodiments, when the interposed layer (non-doped layer) is disposed between the first emitting layer and the second emitting layer in the emitting region, it is expected that a region where the Singlet emitting region and the TTF emitting region overlap with each other is reduced to inhibit a decrease in TTF efficiency caused by collision between triplet excitons and carriers. That is, it is considered that providing the interposed layer (non-doped layer) between the emitting layers contributes to the improvement in the efficiency of TTF emission.

The interposed layer is a non-doped layer.

The interposed layer contains no metal atom. The interposed layer thus contains no metal complex.

The interposed layer contains an interposed layer material. The interposed layer material is not a luminescent compound.

The interposed layer material may be any material except for the luminescent compound.

Examples of the interposed layer material include: 1) a heterocyclic compound such as an oxadiazole derivative, benzimidazole derivative, or phenanthroline derivative; 2) a fused aromatic compound such as a carbazole derivative, anthracene derivative, phenanthrene derivative, pyrene derivative or chrysene derivative; and 3) an aromatic amine compound such as a triarylamine derivative or a fused polycyclic aromatic amine derivative.

At least one of the first host material, the second host material, or the third host material may be used as the interposed layer material. The interposed layer material may be any material provided that the Singlet emitting region and the TTF emitting region are separated from each other and the Singlet emission and the TTF emission are not hindered.

In the organic EL device of each of the exemplary embodiments, the content ratios of all the materials forming the interposed layer in the interposed layer are each 10 mass % or more.

The interposed layer contains the interposed layer material as a material forming the interposed layer.

The interposed layer contains the interposed layer material preferably at 60 mass % or more, more preferably at 70 mass % or more, still more preferably at 80 mass % or more, still further more preferably at 90 mass % or more, and yet still further more preferably at 95 mass % or more, with respect to the total mass of the interposed layer.

The interposed layer may contain a single type of the interposed layer material or may contain two or more types of the interposed layer material.

When the interposed layer contains two or more types of the interposed layer material, an upper limit of the total of the content ratios of the two or more types of the interposed layer material is 100 mass %.

It should be noted that the interposed layer of the organic EL device of each of the exemplary embodiments may further contain any other material than the interposed layer material.

The interposed layer may be provided in the form of a single layer or a laminate of two or more layers.

As long as the overlap between the Singlet emitting region and the TTF emitting region is inhibited, a film thickness of the interposed layer is not particularly limited but each layer in the interposed layer is preferably in a range from 3 nm to 15 nm, more preferably in a range from 5 nm to 10 nm.

The interposed layer having a film thickness of 3 nm or more easily separates the Singlet emitting region from the emitting region derived from TTF.

The interposed layer having a film thickness of 15 nm or less easily inhibits a phenomenon where the host material of the interposed layer emits light.

It is preferable that the interposed layer contains the interposed layer material as a material forming the interposed layer and the triplet energy of the first host material T₁(H1), the triplet energy of the second host material T₁(H2), and a triplet energy of at least one interposed layer material T₁(M_{mid}) satisfy a relationship of a numerical formula (Numerical Formula C21) below.

$${T}_{1} ( H 1 ) \geq {T}_{1} ( {M}_{m i d} ) \geq {T}_{1} ( H 2 ) \qquad ( Numerical Formula C 21 )$$

It is preferable that the interposed layer contains the interposed layer material as a material forming the interposed layer and the triplet energy of the first host material T₁(H1), the triplet energy of the third host material T₁(H3), and the triplet energy of at least one interposed layer material T₁(M_{mid}) satisfy a relationship of a numerical formula (Numerical Formula C22) below.

$${T}_{1} ( H 1 ) \geq {T}_{1} ( {M}_{m i d} ) \geq {T}_{1} ( H 3 ) \qquad ( Numerical Formula C 22 )$$

When the interposed layer contains two or more interposed layer materials as a material forming the interposed layer, the triplet energy of the first host material T₁(H1), the triplet energy of the second host material T₁(H2), and a triplet energy of each interposed layer material T₁(M_{EA}) more preferably satisfy a relationship of a numerical formula (Numerical Formula C23) below.

$${T}_{1} ( H 1 ) \geq {T}_{1} ( {M}_{EA} ) \geq {T}_{1} ( H 2 ) \qquad ( Numerical Formula C 23 )$$

When the interposed layer contains two or more interposed layer materials as a material forming the interposed layer, the triplet energy of the first host material T₁(H1), the triplet energy of the third host material T₁(H3), and the triplet energy of each interposed layer material T₁ (M_{EA}) more preferably satisfy a relationship of a numerical formula (Numerical Formula C24) below.

$${T}_{1} ( H 1 ) \geq {T}_{1} ( {M}_{EA} ) \geq {T}_{1} ( H 3 ) \qquad ( Numerical Formula C 24 )$$

### First Material

In the organic EL device of each of the exemplary embodiments, the first material is not particularly limited as long as the first material satisfies the relationship of the numerical formula (Numerical Formula A1).

In an exemplary arrangement of the organic EL device of each of the exemplary embodiments, the energy level of the highest occupied molecular orbital of the first material HOMO(HT1) satisfies a relationship of a numerical formula (Numerical Formula B1) or a numerical formula (Numerical Formula B2) below.

$$HOMO ( HT 1 ) > - 5 \text{.50} eV \qquad ( Numerical Formula B1 )$$

$$HOMO ( HT 1 ) > - 5.45 eV \qquad ( Numerical Formula B2 )$$

In an exemplary arrangement of the organic EL device of each of the exemplary embodiments, the first material is an amine compound having a substituted amino group.

In an exemplary arrangement of the organic EL device of each of the exemplary embodiments, the first material is an amine compound having a group represented by —N(R_{B1})(R_{B2}), and R_{B1} and R_{B2} are each independently a substituted or unsubstituted aryl group having 6 to 50 ring carbon atoms, or a substituted or unsubstituted heterocyclic group having 5 to 50 ring atoms.

### Additional Layers of Organic EL Device

In addition to the emitting layers described in the exemplary embodiments, the organic EL device of each of the exemplary embodiments may include one or more organic layers. Examples of the organic layer include at least one layer selected from the group consisting of a hole injecting layer, a hole transporting layer, an emitting layer, an electron injecting layer, an electron transporting layer, a hole blocking layer, and an electron blocking layer.

In the organic EL device of each of the exemplary embodiments, the organic layer may consist of the emitting layers described in each of the exemplary embodiments. Alternatively, the organic layer may further include, for instance, at least one layer selected from the group consisting of a hole injecting layer, a hole transporting layer, an electron injecting layer, an electron transporting layer, a hole blocking layer, and an electron blocking layer.

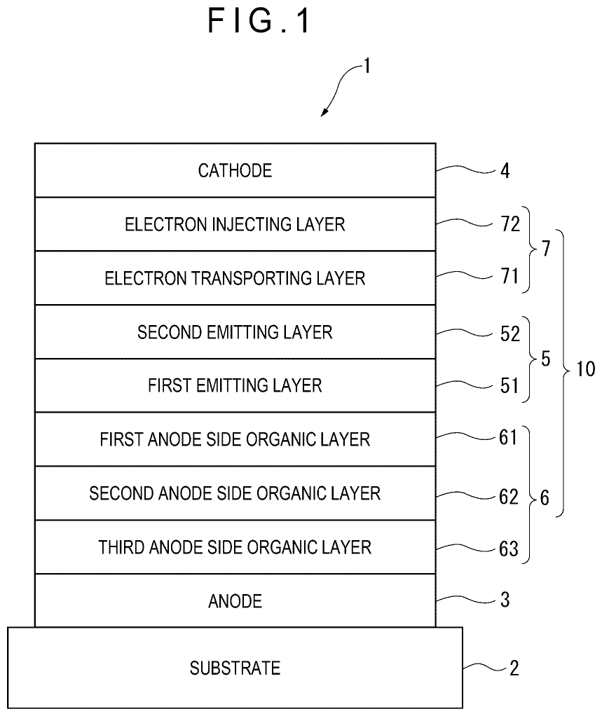

FIG. 1 schematically depicts an exemplary arrangement of an organic EL device according to the first exemplary embodiment.

An organic EL device 1 includes a light-transmissive substrate 2, an anode 3, a cathode 4, and organic layers 10 disposed between the anode 3 and the cathode 4. The organic layers 10 include a first hole transporting zone 6, a first emitting region 5, and a first electron transporting zone 7 in this order from a side close to the anode 3. The first hole transporting zone 6 includes a third anode side organic layer 63, a second anode side organic layer 62, and a first anode side organic layer 61 in this order from a side close to the anode 3. The first emitting region 5 includes a first emitting layer 51 and a second emitting layer 52 in this order from a side close to the anode 3. The first electron transporting zone 7 includes an electron transporting layer 71 and an electron injecting layer 72 in this order from a side close to the first emitting region 5.

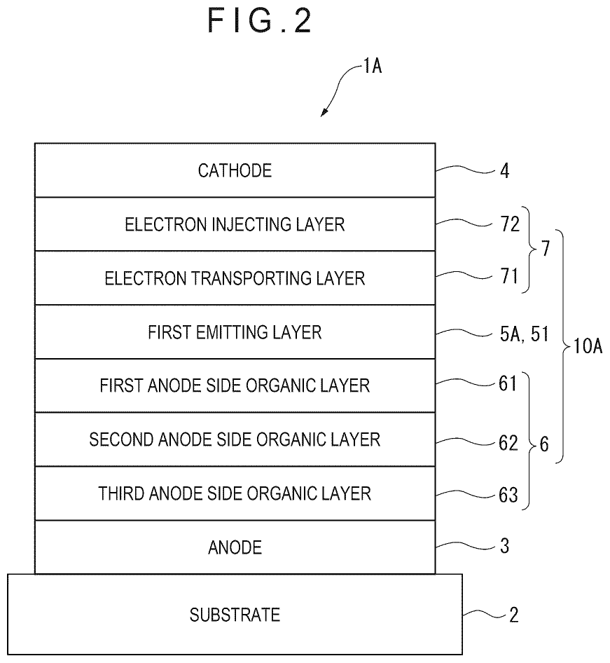

FIG. 2 schematically depicts an exemplary arrangement of an organic EL device according to each of the second, third, and fifth exemplary embodiments.

An organic EL device 1A depicted in FIG. 2 is different from the organic EL device 1 in that organic layers 10A include a first emitting region 5A, and the rest of components and arrangements of the organic EL device 1A are the same as those of the organic EL device 1. The first emitting region 5A includes the first emitting layer 51 as a single emitting layer.

In an exemplary arrangement of the organic EL device of each of the exemplary embodiments, the third anode side organic layer is a hole injecting layer.

In an exemplary arrangement of the organic EL device of each of the exemplary embodiments, the second anode side organic layer is a hole transporting layer.

In an exemplary arrangement of the organic EL device of each of the exemplary embodiments, the first anode side organic layer is an electron blocking layer.

The invention is not limited to the exemplary arrangements of the organic EL devices depicted in FIGS. 1 to 2.

The arrangement of the organic EL device of each of the exemplary embodiments will be further described below. It should be noted that the reference numerals are occasionally omitted below.

### Substrate

The substrate is used as a support for the organic EL device. For instance, glass, quartz, plastics and the like are usable for the substrate. A flexible substrate is also usable. The flexible substrate, which is a bendable substrate, is exemplified by a plastic substrate. Examples of a material for the plastic substrate include polycarbonate, polyarylate, polyethersulfone, polypropylene, polyester, polyvinyl fluoride, polyvinyl chloride, polyimide, and polyethylene naphthalate. Further, an inorganic vapor deposition film is also usable.

### Anode

Metal, an alloy, an electrically conductive compound, a mixture thereof, or the like having a large work function (specifically, 4.0 eV or more) is preferably used as the anode formed on the substrate. Specific examples of the material include indium tin oxide (ITO), indium oxide-tin oxide containing silicon or silicon oxide, indium oxide-zinc oxide, indium oxide containing tungsten oxide and zinc oxide, and graphene. In addition, gold (Au), platinum (Pt), nickel (Ni), tungsten (W), chrome (Cr), molybdenum (Mo), iron (Fe), cobalt (Co), copper (Cu), palladium (Pd), titanium (Ti), and nitrides of a metal material (e.g., titanium nitride) are usable.

The material is typically formed into a film by a sputtering method. For instance, the indium oxide-zinc oxide can be formed into a film by the sputtering method using a target in which zinc oxide in a range from 1 mass % to 10 mass % is added to indium oxide. Moreover, for instance, the indium oxide containing tungsten oxide and zinc oxide can be formed by the sputtering method using a target in which tungsten oxide in a range from 0.5 mass % to 5 mass % and zinc oxide in a range from 0.1 mass % to 1 mass % are added to indium oxide. In addition, the anode may be formed by a vacuum deposition method, a coating method, an inkjet method, a spin coating method or the like.

Among the EL layers formed on the anode, since the hole injecting layer adjacent to the anode is formed of a composite material into which holes are easily injectable irrespective of the work function of the anode, a material usable as an electrode material (e.g., metal, an alloy, an electroconductive compound, a mixture thereof, and the elements belonging to the group 1 or 2 of the periodic table) is also usable for the anode.

A material having a small work function such as elements belonging to Groups 1 and 2 in the periodic table of the elements, specifically, an alkali metal such as lithium (Li) and cesium (Cs), an alkaline earth metal such as magnesium (Mg), calcium (Ca) and strontium (Sr), alloys (e.g., MgAg and AlLi) including the alkali metal or the alkaline earth metal, a rare earth metal such as europium (Eu) and ytterbium (Yb), alloys including the rare earth metal are also usable for the anode. It should be noted that the vacuum deposition method and the sputtering method are usable for forming the anode using the alkali metal, alkaline earth metal and the alloy thereof. Further, when a silver paste is used for the anode, the coating method and the inkjet method are usable.

### Cathode

It is preferable to use metal, an alloy, an electroconductive compound, a mixture thereof, or the like having a small work function (specifically, 3.8 eV or less) for the cathode. Examples of the material for the cathode include elements belonging to Groups 1 and 2 in the periodic table of the elements, specifically, an alkali metal such as lithium (Li) and cesium (Cs), an alkaline earth metal such as magnesium (Mg), calcium (Ca) and strontium (Sr), alloys (e.g., MgAg and AlLi) including the alkali metal or the alkaline earth metal, a rare earth metal such as europium (Eu) and ytterbium (Yb), and alloys including the rare earth metal.

It should be noted that the vacuum deposition method and the sputtering method are usable for forming the cathode using the alkali metal, alkaline earth metal and the alloy thereof. Further, when a silver paste is used for the cathode, the coating method and the inkjet method are usable.

By providing the electron injecting layer, various conductive materials such as A1, Ag, ITO, graphene, and indium oxide-tin oxide containing silicon or silicon oxide may be used for forming the cathode regardless of the work function. The conductive materials can be formed into a film using the sputtering method, inkjet method, spin coating method, and the like.

### Hole Injecting Layer

The hole injecting layer is a layer containing a substance exhibiting a high hole injectability. Examples of the substance exhibiting a high hole injectability include molybdenum oxide, titanium oxide, vanadium oxide, rhenium oxide, ruthenium oxide, chrome oxide, zirconium oxide, hafnium oxide, tantalum oxide, silver oxide, tungsten oxide, and manganese oxide.

In addition, the examples of the substance exhibiting a high hole injectability include: aromatic amine compounds such as 4,4′,4″-tris(N,N-diphenylamino)triphenylamine (abbreviation: TDATA), 4,4′,4″-tris[N-(3-methylphenyl)-N-phenylamino]triphenylamine (abbreviation: MTDATA), 4,4′-bis[N-(4-diphenylaminophenyl)-N-phenylamino]biphenyl (abbreviation: DPAB), 4,4′-bis(N-{4-[N′-(3-methylphenyl)-N′-phenylamino]phenyl}-N-phenylamino) biphenyl (abbreviation: DNTPD), 1,3,5-tris[N-(4-diphenylaminophenyl)-N-phenylamino]benzene (abbreviation: DPA3B), 3-[N-(9-phenylcarbazole-3-yl)-N-phenylamino]-9-phenylcarbazole (abbreviation: PCzPCA1), 3,6-bis[N-(9-phenylcarbazole-3-yl)-N-phenylamino]-9-phenylcarbazole (abbreviation: PCzPCA2), and 3-[N-(1-naphthyl)-N-(9-phenylcarbazole-3-yl)amino]-9-phenylcarbazole (abbreviation: PCzPCN1); and dipyrazino[2,3-f: 20,30-h]quinoxaline-2,3,6,7,10,11-hexacarbonitrile (HAT-CN), those of which are low-molecule organic compounds.

In addition, a high polymer compound (e.g., oligomer, dendrimer and polymer) is usable as the substance exhibiting a high hole injectability. Examples of the high polymer compound include poly(N-vinylcarbazole) (abbreviation: PVK), poly(4-vinyltriphenylamine) (abbreviation: PVTPA), poly[N-(4-{N′-[4-(4-diphenylamino)phenyl]phenyl-N′-phenylamino}phenyl) methacrylamide] (abbreviation: PTPDMA), and poly[N,N′-bis(4-butylphenyl)-N, N′-bis(phenyl) benzidine] (abbreviation: Poly-TPD). Moreover, an acid-added high polymer compound such as poly(3,4-ethylenedioxythiophene)/poly(styrene sulfonic acid) (PEDOT/PSS) and polyaniline/poly(styrene sulfonic acid) (PAni/PSS) is also usable.

### Hole Transporting Layer

In an exemplary arrangement of the organic EL device according to each of the exemplary embodiments, the hole transporting layer is disposed between the anode and the emitting region.

The hole transporting layer is a layer containing a substance exhibiting a high hole transportability. An aromatic amine compound, carbazole derivative, anthracene derivative and the like are usable for the hole transporting layer. Specific examples of a material for the hole transporting layer include an aromatic amine compound such as 4,4′-bis[N-(1-naphthyl)-N-phenylamino]biphenyl (abbreviation: NPB), N,N′-bis(3-methylphenyl)-N, N′-diphenyl-[1,1′-biphenyl]-4,4′-diamine (abbreviation: TPD), 4-phenyl-4′-(9-phenylfluorene-9-yl)triphenylamine (abbreviation: BAFLP), 4,4′-bis[N-(9,9-dimethylfluorene-2-yl)-N-phenylamino]biphenyl (abbreviation: DFLDPBi), 4,4′,4″-tris(N,N-diphenylamino) triphenylamine (abbreviation: TDATA), 4,4′,4″-tris[N-(3-methylphenyl)-N-phenylamino]triphenylamine (abbreviation: MTDATA), and 4,4′-bis[N-(spiro-9,9′-bifluorene-2-yl)-N-phenylamino]biphenyl (abbreviation: BSPB). The above-described substances mostly have a hole mobility of 10⁻⁶ cm²/(V·s) or more.

For the hole transporting layer, a carbazole derivative such as CBP, 9-[4-(N-carbazolyl)]phenyl-10-phenylanthracene (CzPA), and 9-phenyl-3-[4-(10-phenyl-9-anthryl)phenyl]-9H-carbazole (PCzPA) and an anthracene derivative such as t-BuDNA, DNA, and DPAnth may be used. A high polymer compound such as poly(N-vinylcarbazole) (abbreviation: PVK) and poly(4-vinyltriphenylamine) (abbreviation: PVTPA) is also usable.

However, in addition to the above substances, any substance exhibiting a higher hole transportability than an electron transportability may be used. It should be noted that the layer containing the substance exhibiting a high hole transportability may be not only a single layer but also a laminate of two or more layers formed of the above substance(s).

### Electron Transporting Layer

In an exemplary arrangement of the organic EL device according to each of the exemplary embodiments, the electron transporting layer is disposed between the cathode and the emitting region.

The electron transporting layer is a layer containing a highly electron-transportable substance. For the electron transporting layer, 1) a metal complex such as an aluminum complex, beryllium complex, and zinc complex, 2) a hetero aromatic compound such as imidazole derivative, benzimidazole derivative, azine derivative, carbazole derivative, and phenanthroline derivative, and 3) a high polymer compound are usable. Specifically, as a low-molecule organic compound, a metal complex such as Alq, tris(4-methyl-8-quinolinolato)aluminum (abbreviation: Almq₃), bis(10-hydroxybenzo[h]quinolinato) beryllium (abbreviation: BeBq2), BAlq, Znq, ZnPBO and ZnBTZ is usable. In addition to the metal complex, a heteroaromatic compound such as 2-(4-biphenylyl)-5-(4-tert-butylphenyl)-1,3,4-oxadiazole (abbreviation: PBD), 1,3-bis[5-(ptert-butylphenyl)-1,3,4-oxadiazole-2-yl]benzene (abbreviation: OXD-7), 3-(4-tert-butylphenyl)-4-phenyl-5-(4-biphenylyl)-1,2,4-triazole (abbreviation: TAZ), 3-(4-tert-butylphenyl)-4-(4-ethylphenyl)-5-(4-biphenylyl)-1,2,4-triazole (abbreviation: p-EtTAZ), bathophenanthroline (abbreviation: BPhen), bathocuproine (abbreviation: BCP), and 4,4′-bis(5-methylbenzoxazole-2-yl) stilbene (abbreviation: BzOs) is usable. Examples of the azine derivative include a pyridine derivative, a pyrimidine derivative and a triazine derivative. In the exemplary embodiment, an azine derivative or a benzimidazole compound is suitably usable. The above-described substances mostly have an electron mobility of 10⁻⁶ cm²/(V·s) or more. It should be noted that any other substance than the above substances may be used for the electron transporting layer as long as the substance exhibits a higher electron transportability than the hole transportability. The electron transporting layer may be a single layer or a laminate of two or more layers formed of the above substance(s).

Further, a high polymer compound is usable for the electron transporting layer. For instance, poly[(9,9-dihexylfluorene-2,7-diyl)-co-(pyridine-3,5-diyl)] (abbreviation: PF-Py), and poly[(9,9-dioctylfluorene-2,7-diyl)-co-(2,2′-bipyridine-6,6′-diyl)] (abbreviation: PF-BPy) are usable.

### Electron Injecting Layer

The electron injecting layer is a layer containing a highly electron-injectable substance. Examples of a material for the electron injecting layer include an alkali metal, alkaline earth metal and a compound thereof, examples of which include lithium (Li), cesium (Cs), calcium (Ca), lithium fluoride (LiF), cesium fluoride (CsF), calcium fluoride (CaF₂), and lithium oxide (LiOx). In addition, the alkali metal, alkaline earth metal or the compound thereof may be added to the substance exhibiting the electron transportability in use. Specifically, for instance, magnesium (Mg) added to Alq may be used. In this case, the electrons can be more efficiently injected from the cathode.

Alternatively, the electron injecting layer may be provided by a composite material in a form of a mixture of the organic compound and the electron donor. Such a composite material exhibits excellent electron injectability and electron transportability since electrons are generated in the organic compound by the electron donor. In this case, the organic compound is preferably a material excellent in transporting the generated electrons. Specifically, the above examples (e.g., the metal complex and the hetero aromatic compound) of the substance forming the electron transporting layer are usable. As the electron donor, any substance exhibiting electron donating property to the organic compound is usable. Specifically, the electron donor is preferably alkali metal, alkaline earth metal and rare earth metal such as lithium, cesium, magnesium, calcium, erbium and ytterbium. The electron donor is also preferably alkali metal oxide and alkaline earth metal oxide such as lithium oxide, calcium oxide, and barium oxide. Moreover, a Lewis base such as magnesium oxide is usable. Further, the organic compound such as tetrathiafulvalene (abbreviation: TTF) is usable.

### Layer Formation Method

A method of forming each layer of the organic EL device in each of the exemplary embodiments is subject to no limitation except for the above particular description. However, known methods of dry film-forming such as vacuum deposition, sputtering, plasma or ion plating and wet film-forming such as spin coating, dipping, flow coating or ink-jet are applicable.

In the organic EL device of each of the exemplary embodiments, the first emitting layer that contains the first host material, the second host material, and the first luminescent compound and the second emitting layer that contains the third host material and the second luminescent compound are formable using the plurality of compounds by co-deposition; are formable, by vapor-deposition, using a mixture obtained by mixing in advance (premixing) the plurality of compounds; or formable, by coating, using a mixture obtained by mixing in advance (premixing) the plurality of compounds. The mixture obtained by mixing in advance (premixing) the plurality of compounds may be a mixture powder. The mixture obtained by mixing in advance (premixing) the plurality of compounds may be a solution. The mixture used for forming the emitting layer may be, for instance, the composition described in the third or fourth exemplary embodiment. A method of mixing in advance a plurality of compounds is occasionally referred to as premix. The premix method is not particularly limited. For instance, a vapor-deposition ratio of compounds forming the mixture obtained by premix is adjustable by adjusting a substituent(s) or the like of the compound(s) forming the mixture to adjust a molecular weight of the compound(s) or adjusting its mixing ratio.

### Film Thickness

The film thickness of each organic layer of the organic EL device in each of the exemplary embodiments is not limited unless otherwise specified in the above. In general, the thickness preferably ranges from several nanometers to 1 μm because an excessively small film thickness is likely to cause defects (e.g. pin holes) and an excessively large thickness leads to the necessity of applying high voltage and consequent reduction in efficiency.

### Form of Composition

The form of the composition in each of the exemplary embodiments is not particularly limited, and exemplified by a solid, powder, solution, and film. The composition in the form of a solid may be formed into a pellet shape. The composition in each of the exemplary embodiments is usable as a mixture mixed in advance (mixture obtained by premix). A film may be formed using the mixture obtained by premix.

The film as one of the forms of the composition described above includes a film formed from a material that contains the first compound and the second compound. Such a film is exemplified by the first emitting layer that contains the first host material and the second host material in the first exemplary embodiment.

### Mixture Powder

In an exemplary arrangement of the composition of each of the exemplary embodiments, the composition is in the form of a mixture powder. The composition according to the third exemplary embodiment may be a mixture powder. The composition according to the fourth exemplary embodiment may be a mixture powder.

A mixture powder according to an exemplary embodiment contains a first compound and a second compound, in which the first compound and the second compound are mutually different compounds, and the first compound and the second compound each independently include, in a molecule, at least one of the structure of Condition (i) or the structure of Condition (ii) in the third exemplary embodiment.

A mixture powder according to an exemplary embodiment contains a first compound and a second compound, in which the first compound and the second compound are mutually different compounds, an energy level of a highest occupied molecular orbital of the first compound HOMO(C1) and an energy level of a highest occupied molecular orbital of the second compound HOMO(C2) satisfy the relationship of the numerical formula (Numerical Formula A7) in the fourth exemplary embodiment, a triplet energy of the first compound T₁(C1) satisfies the relationship of the numerical formula (Numerical Formula A8), and a triplet energy of the second compound T₁(C2) satisfies the relationship of the numerical formula (Numerical Formula A9).

The mixture powder according to the exemplary embodiment is usable for the organic EL device. The organic EL device in which the mixture powder according to the exemplary embodiment is used has improved device performance.

The emitting layer of the organic EL device can be formed by using the mixture powder according to the exemplary embodiment. Forming the emitting layer of the organic EL device by use of the mixture powder according to the exemplary embodiment allows the organic EL device to maintain luminous efficiency and have a long lifetime.

Compound ratio of Compounds in Composition

In an exemplary arrangement of the composition of each of the exemplary embodiments, a mass percentage of a mass of the second compound M_{C2} with respect to a total M_{C1}+M_{C2} of a mass of the first compound M_{C1} and the mass of the second compound M_{C2} in the composition is in a range from 0.5 mass % to 60 mass %.

In an exemplary arrangement of the composition of each of the exemplary embodiments, the mass percentage of the mass of the second compound M_{C2} with respect to the total M_{C1}+M_{C2} is 2 mass % or more, 10 mass % or more, 25 mass % or more, or 40 mass % or more.

In an exemplary arrangement of the composition of each of the exemplary embodiments, the mass percentage of the mass of the second compound M_{C2} with respect to the total M_{C1}+M_{C2} is 55 mass % or less.

### Measurement Method

### Energy Level of Highest Occupied Molecular Orbital HOMO

A value of the energy level of the highest occupied molecular orbital HOMO of a measurement target (compound or material) is calculated by a numerical formula (Numerical Formula F3) below. The unit of the energy level of the highest occupied molecular orbital HOMO is eV.

$$HOMO = - 1.4 \times ( Eox - Efc ) - 4.6 eV \qquad ( F3 )$$

In the numerical formula (Numerical Formula F3), Eox and Efc are as follows:
Eox: first oxidation potential (DPV, positive scan)
Efc: first oxidation potential of ferrocene (DPV, positive scan), (ca. +0.55 V vs Ag/AgCl)

Oxidation-reduction potential is measured by the differential pulse voltammetry (DPV) method using an electrochemical analyzer (CHI852D produced by ALS).

A sample solution used for the measurement is prepared by dissolving the measurement target and a supporting electrolyte in a solvent. In the sample solution, the concentrations of the measurement target and the supporting electrolyte are 1.0 mmol/L and 100 mmol/L, respectively. N, N-dimethylformamide (DMF) is used as the solvent. Tetrabutylammmonium hexafluorophosphate (TBHP) is used as the supporting electrolyte. A glassy carbon electrode is used as a working electrode. A platinum (Pt) electrode is used as a counter electrode. Reference Literature 1 (M. E. Thompson, et. al., Organic Electronics, 6 (2005), pp. 11-20) and Reference Literature 2 (Organic Electronics, 10 (2009), pp. 515-520) are exemplified as documents regarding the measurement of the energy level of the highest occupied molecular orbital HOMO.

### Triplet Energy T₁

A method of measuring a triplet energy T₁ is exemplified by a method below.

A measurement target compound is dissolved in EPA (diethylether:isopentane:ethanol=5:5:2 in volume ratio) so as to fall within a range from 10⁻⁵ mol/L to 10⁻⁴ mol/L to prepare a solution, and the obtained solution is encapsulated in a quartz cell to provide a measurement sample. A phosphorescence spectrum (ordinate axis: phosphorescent luminous intensity, abscissa axis: wavelength) of the measurement sample is measured at a low temperature (77K). A tangent is drawn to the rise of the phosphorescence spectrum close to the short-wavelength region. An energy amount is calculated by a conversion equation (F1) below on a basis of a wavelength value λ_{edge} [nm] at an intersection of the tangent and the abscissa axis. The calculated energy amount is defined as triplet energy T₁.

$${T}_{1} [ eV ] = 1239.85 / {λ}_{edge} \qquad Conversion Equation ( F 1 )$$

The tangent to the rise of the phosphorescence spectrum close to the short-wavelength region is drawn as follows. While moving on a curve of the phosphorescence spectrum from the short-wavelength region to the local maximum value closest to the short-wavelength region among the local maximum values of the phosphorescence spectrum, a tangent is checked at each point on the curve toward the long-wavelength region of the phosphorescence spectrum. An inclination of the tangent is increased along the rise of the curve (i.e., a value of the ordinate axis is increased). A tangent drawn at a point of the local maximum inclination (i.e., a tangent at an inflection point) is defined as the tangent to the rise of the phosphorescence spectrum close to the short-wavelength region.

A local maximum point where a peak intensity is 15% or less of the maximum peak intensity of the spectrum is not counted as the above-mentioned local maximum peak intensity closest to the short-wavelength region. The tangent drawn at a point that is closest to the local maximum peak intensity closest to the short-wavelength region and where the inclination of the curve is the local maximum is defined as a tangent to the rise of the phosphorescence spectrum close to the short-wavelength region.

For phosphorescence measurement, a spectrophotofluorometer body F-4500 (produced by Hitachi High-Technologies Corporation) is usable. The measurement apparatus is not limited thereto. A combination of a cooling unit, a low temperature container, an excitation light source and a light-receiving unit may be used for measurement.

### Singlet Energy S₁

A method of measuring a singlet energy S₁ with use of a solution (occasionally referred to as a solution method) is exemplified by a method below.

A toluene solution of a measurement target compound at a concentration ranging from 10⁻⁵ mol/L to 10⁻⁴ mol/L is prepared and put in a quartz cell. An absorption spectrum (ordinate axis: absorption intensity, abscissa axis: wavelength) of the thus-obtained sample is measured at a normal temperature (300K). A tangent is drawn to the fall of the absorption spectrum close to the long-wavelength region, and a wavelength value λ_{edge} (nm) at an intersection of the tangent and the abscissa axis is assigned to a conversion equation (F2) below to calculate a singlet energy.

$$Conversion Equation ( F 2 ) : {S}_{1} [ eV ] = 1239 \text{.85} / λ edge$$

Any apparatus for measuring the absorption spectrum is usable. For instance, a spectrophotometer (U3310 produced by Hitachi, Ltd.) is usable.

The tangent to the fall of the absorption spectrum close to the long-wavelength region is drawn as follows. While moving on a curve of the absorption spectrum from the local maximum value closest to the long-wavelength region, among the local maximum values of the absorption spectrum, in a long-wavelength direction, a tangent at each point on the curve is checked. An inclination of the tangent is decreased and increased in a repeated manner as the curve falls (i.e., a value of the ordinate axis is decreased). A tangent drawn at a point where the inclination of the curve is the local minimum closest to the long-wavelength region (except when absorbance is 0.1 or less) is defined as the tangent to the fall of the absorption spectrum close to the long-wavelength region.

The local maximum absorbance of 0.2 or less is not counted as the above-mentioned local maximum absorbance closest to the long-wavelength region.

### Maximum Peak Wavelength of Compound

A method of measuring the maximum peak wavelength of the compound is as follows. A toluene solution of a measurement target compound at a concentration of 5 μmol/L was prepared and put in a quartz cell. An emission spectrum (ordinate axis: luminous intensity, abscissa axis: wavelength) of the thus-obtained sample was measured at a normal temperature (300K). The emission spectrum can be measured using a spectrophotometer (apparatus name: F-7000) produced by Hitachi High-Tech Science Corporation. It should be noted that the apparatus for measuring the emission spectrum is not limited to the apparatus used herein.

A peak wavelength of the emission spectrum exhibiting the maximum luminous intensity is defined as the maximum peak wavelength. Herein, the maximum peak wavelength is occasionally referred to as a maximum fluorescence peak wavelength (FL-peak).

### Sixth Exemplary Embodiment

### Electronic Device

An electronic device according to a sixth exemplary embodiment is installed with the organic electroluminescence device according to any one of the above exemplary embodiments. Examples of the electronic device include a display device and a light-emitting unit. Examples of the display device include a display component (e.g., an organic EL panel module), TV, mobile phone, tablet and personal computer. Examples of the light-emitting unit include an illuminator and a vehicle light.

In an exemplary arrangement of the electronic device according to the sixth exemplary embodiment, the display device is installed with the light-emitting unit of the exemplary embodiment. The light-emitting unit can be also used for the display device, for instance, as a backlight of the display device.

### Modification of Embodiments

The scope of the invention is not limited to the above exemplary embodiments but includes any modification and improvement as long as such modification and improvement are compatible with the invention.

For instance, the number of emitting layers is not limited to one or two, and three or more emitting layers may be layered. The rest of the emitting layers different from the emitting layers described in the above exemplary embodiments may be a fluorescent emitting layer or a phosphorescent emitting layer with use of emission caused by electron transfer from the triplet excited state directly to the ground state.

When the organic EL device includes a plurality of emitting layers, these emitting layers may be mutually adjacently provided, or may form a so-called tandem organic EL device in which a plurality of emitting units are layered via an intermediate layer.

For instance, a blocking layer may be provided adjacent to at least one of a side of the emitting layer close to the anode or a side of the emitting layer close to the cathode. The blocking layer is preferably provided in contact with the emitting layer to block at least one of holes, electrons, or excitons.

For instance, when the blocking layer is provided in contact with the side of the emitting layer close to the cathode, the blocking layer permits transport of electrons, and blocks holes from reaching a layer provided closer to the cathode (e.g., the electron transporting layer) beyond the blocking layer. When the organic EL device includes the electron transporting layer, the blocking layer is preferably interposed between the emitting layer and the electron transporting layer.

When the blocking layer is provided in contact with the side of the emitting layer close to the anode, the blocking layer permits transport of holes and blocks electrons from reaching a layer provided closer to the anode (e.g., the hole transporting layer) beyond the blocking layer. When the organic EL device includes the hole transporting layer, the blocking layer is preferably interposed between the emitting layer and the hole transporting layer.

Alternatively, the blocking layer may be provided adjacent to the emitting layer so that the excitation energy does not leak out from the emitting layer toward neighboring layer(s). The blocking layer blocks excitons generated in the emitting layer from being transferred to a layer(s) (e.g., the electron transporting layer and the hole transporting layer) closer to the electrode(s) beyond the blocking layer.

The emitting layer is preferably bonded with the blocking layer.

The specific structure, shape, and the like of the components in the invention may be designed in any manner as long as an object of the invention can be achieved.

### EXAMPLES

The invention will be described in further detail with reference to Examples. The scope of the invention is by no means limited to Examples.

### Compounds

Structures of compounds, as the first host material or the second host material, used for producing organic EL devices in Examples 1 to 31 and Comparative Examples 1 to 24 are given below.

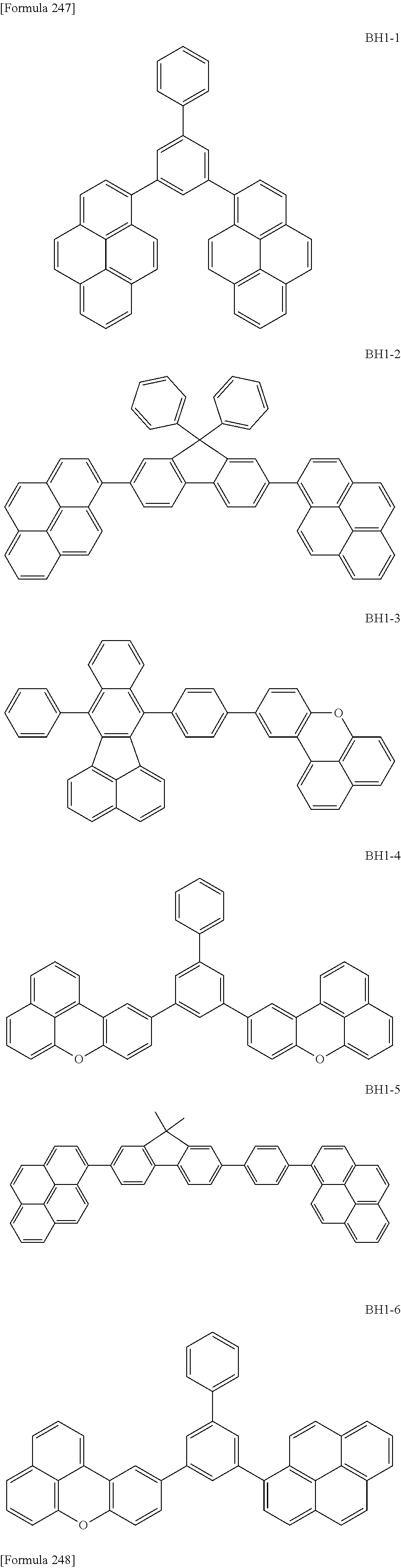

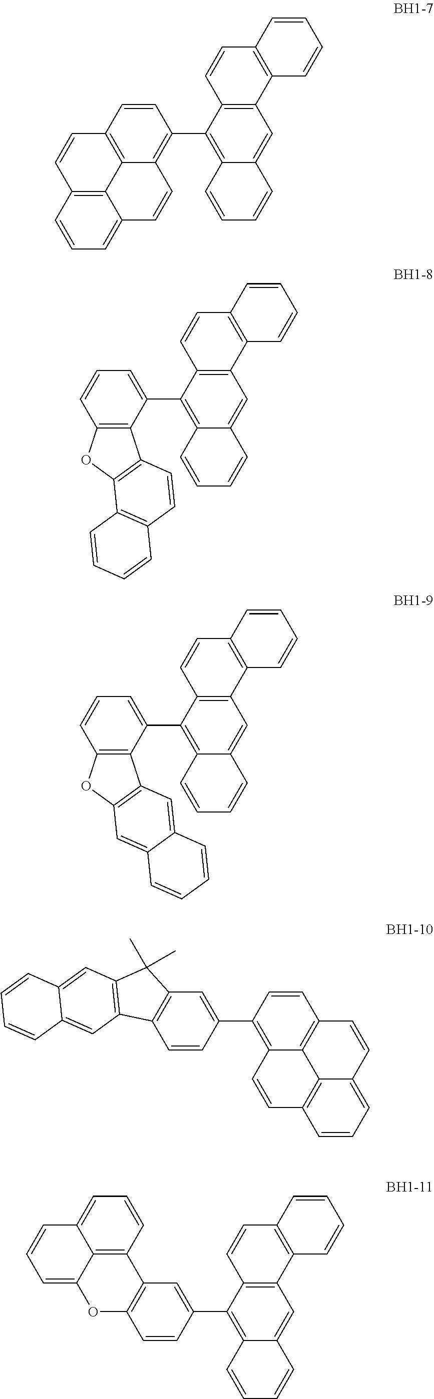

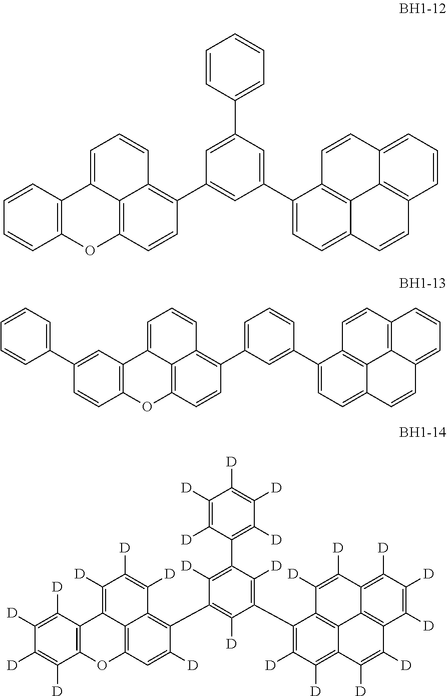 [SMILES: c1ccc(-c2cc(-c3ccc4c5c(cccc35)-c3ccccc3O4)cc(-c3ccc4ccc5cccc6ccc3c4c56)c2)cc1 ; c1ccc(-c2ccc3c(c2)-c2cccc4c(-c5cccc(-c6ccc7ccc8cccc9ccc6c7c89)c5)ccc(c24)O3)cc1 ; [2H]c1cc2c3c(c([2H])c([2H])c([2H])c3c1-c1c([2H])c(-c3c([2H])c([2H])c([2H])c([2H])c3[2H])c([2H])c(-c3c([2H])c([2H])c4c([2H])c([2H])c5c([2H])c([2H])c([2H])c6c([2H])c([2H])c3c4c56)c1[2H])-c1c([2H])c([2H])c([2H])c([2H])c1O2]

Structures of other compounds used for producing organic EL devices in Examples 1 to 31 and Comparative Examples 1 to 24 are given below.

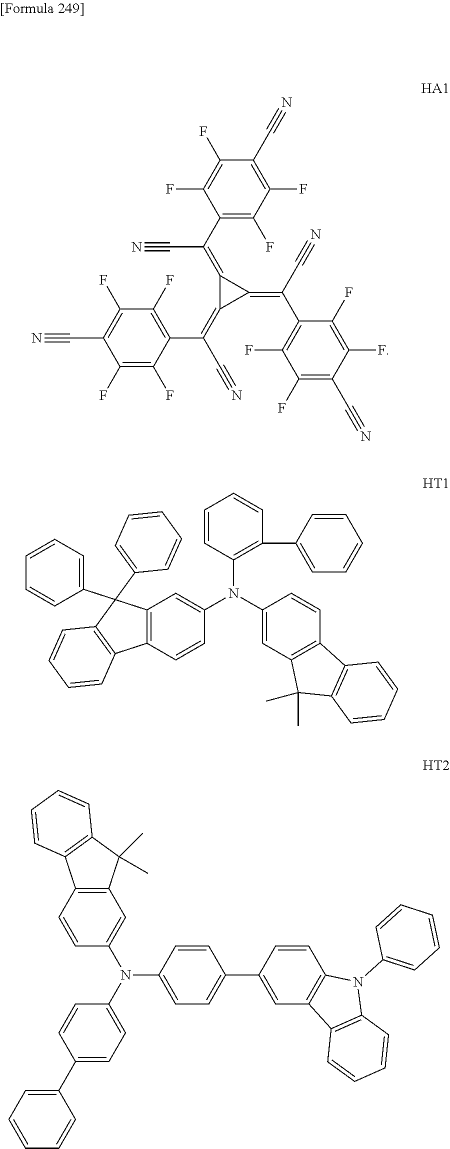 [SMILES: N#CC(=C1C(=C(C#N)c2c(F)c(F)c(C#N)c(F)c2F)C1=C(C#N)c1c(F)c(F)c(C#N)c(F)c1F)c1c(F)c(F)c(C#N)c(F)c1F ; CC1(C)c2ccccc2-c2ccc(N(c3ccc4c(c3)C(c3ccccc3)(c3ccccc3)c3ccccc3-4)c3ccccc3-c3ccccc3)cc21 ; CC1(C)c2ccccc2-c2ccc(N(c3ccc(-c4ccccc4)cc3)c3ccc(-c4ccc5c(c4)c4ccccc4n5-c4ccccc4)cc3)cc21]

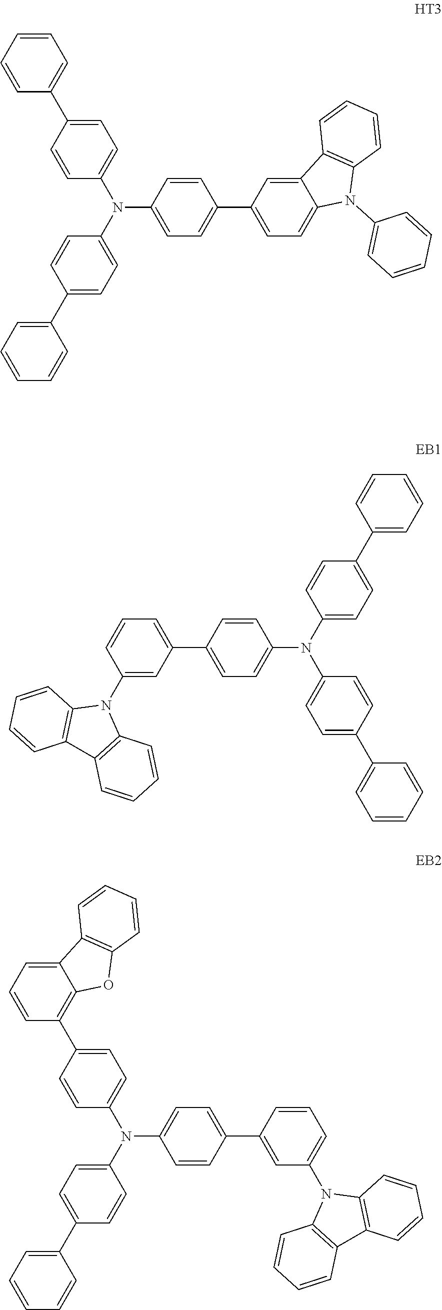 [SMILES: c1ccc(-c2ccc(N(c3ccc(-c4ccccc4)cc3)c3ccc(-c4ccc5c(c4)c4ccccc4n5-c4ccccc4)cc3)cc2)cc1 ; c1ccc(-c2ccc(N(c3ccc(-c4ccccc4)cc3)c3ccc(-c4cccc(-n5c6ccccc6c6ccccc65)c4)cc3)cc2)cc1 ; c1ccc(-c2ccc(N(c3ccc(-c4cccc(-n5c6ccccc6c6ccccc65)c4)cc3)c3ccc(-c4cccc5c4oc4ccccc45)cc3)cc2)cc1]

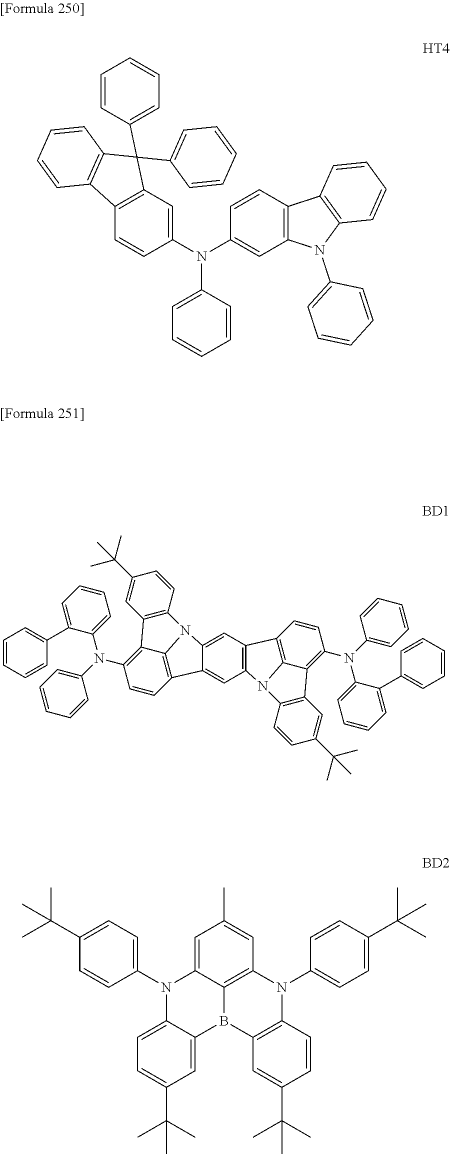 [SMILES: c1ccc(N(c2ccc3c(c2)C(c2ccccc2)(c2ccccc2)c2ccccc2-3)c2ccc3c4ccccc4n(-c4ccccc4)c3c2)cc1 ; CC(C)(C)c1ccc2c(c1)c1c(N(c3ccccc3)c3ccccc3-c3ccccc3)ccc3c4cc5c(cc4n2c31)c1ccc(N(c2ccccc2)c2ccccc2-c2ccccc2)c2c3cc(C(C)(C)C)ccc3n5c12 ; Cc1cc2c3c(c1)N(c1ccc(C(C)(C)C)cc1)c1ccc(C(C)(C)C)cc1B3c1cc(C(C)(C)C)ccc1N2c1ccc(C(C)(C)C)cc1]

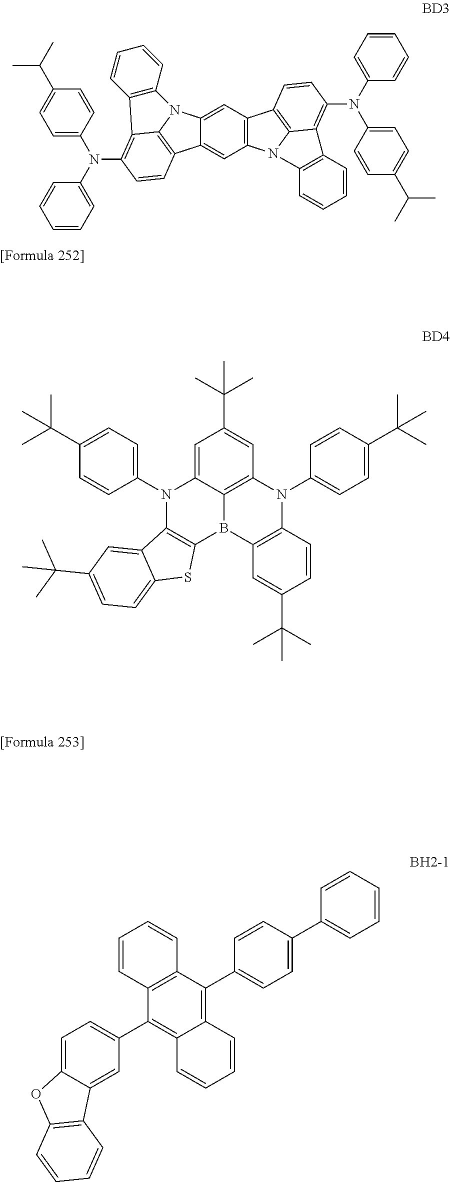

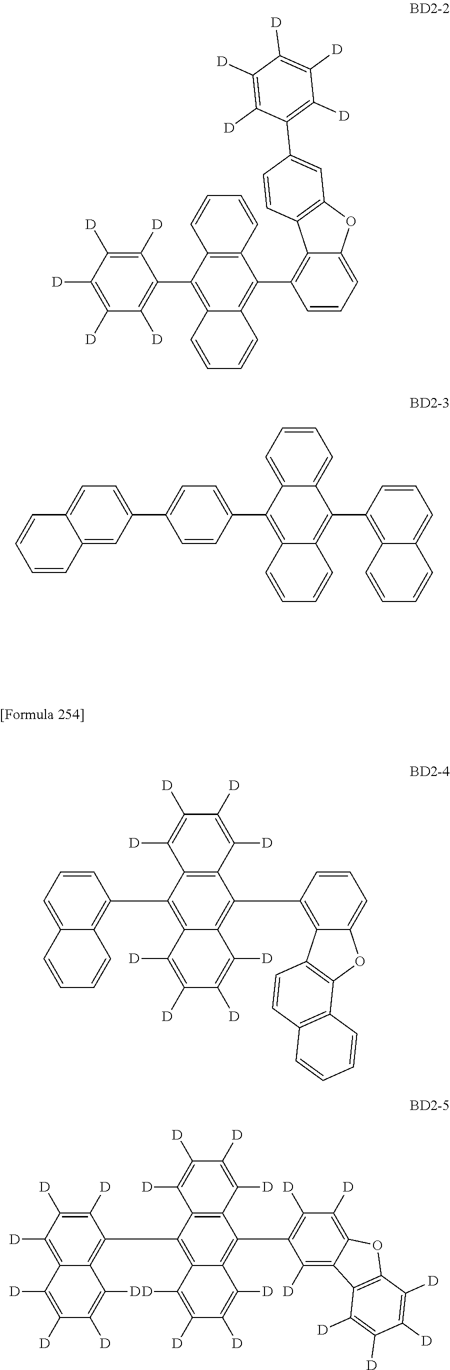

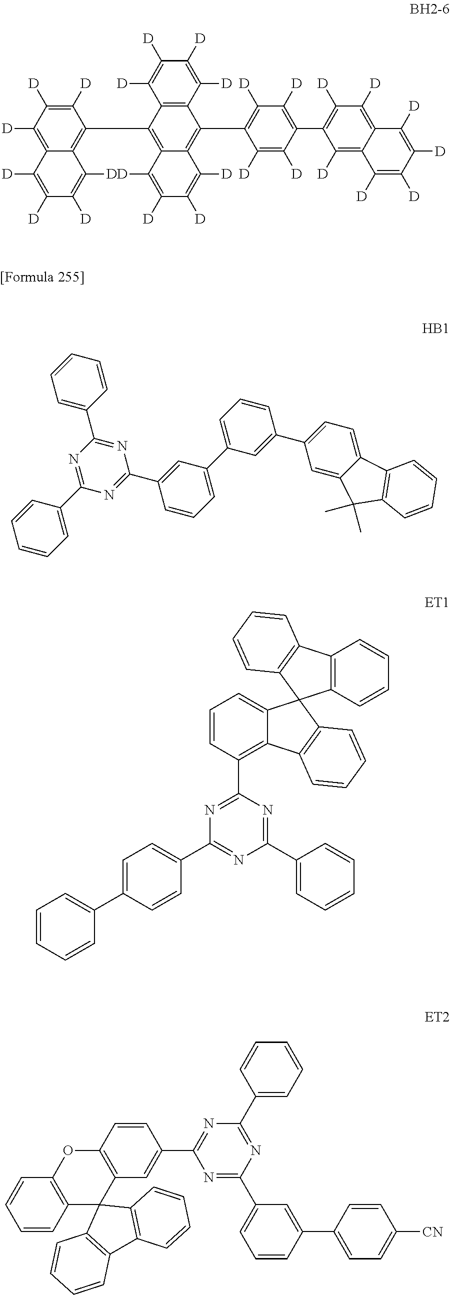 [SMILES: [2H]c1c([2H])c(-c2c3c([2H])c([2H])c([2H])c([2H])c3c(-c3c([2H])c([2H])c([2H])c4c([2H])c([2H])c([2H])c([2H])c34)c3c([2H])c([2H])c([2H])c([2H])c23)c([2H])c([2H])c1-c1c([2H])c([2H])c2c([2H])c([2H])c([2H])c([2H])c2c1[2H] ; CC1(C)c2ccccc2-c2ccc(-c3cccc(-c4cccc(-c5nc(-c6ccccc6)nc(-c6ccccc6)n5)c4)c3)cc21 ; c1ccc(-c2ccc(-c3nc(-c4ccccc4)nc(-c4cccc5c4-c4ccccc4C54c5ccccc5-c5ccccc54)n3)cc2)cc1 ; N#Cc1ccc(-c2cccc(-c3nc(-c4ccccc4)nc(-c4ccc5c(c4)C4(c6ccccc6O5)c5ccccc5-c5ccccc54)n3)c2)cc1]

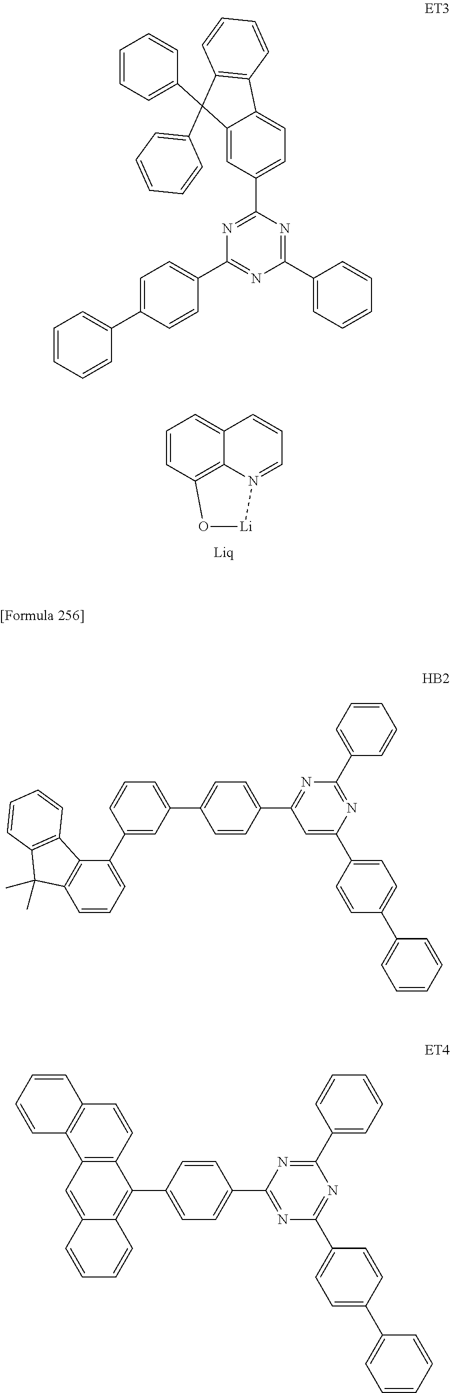

### Production (1) of Organic EL Device

The organic EL devices were produced as follows.

### Example 1

A glass substrate (size: 25 mm×75 mm×1.1 mm thick, produced by Geomatec Co., Ltd.) having an indium tin oxide (ITO) transparent electrode (anode) was ultrasonic-cleaned in isopropyl alcohol for five minutes, and then UV-ozone-cleaned for 30 minutes. The film thickness of the ITO transparent electrode was 130 nm.

After the glass substrate having the transparent electrode line was cleaned, the glass substrate was mounted on a substrate holder of a vacuum deposition apparatus. First, a compound HT1 and a compound HA1 were co-deposited on a surface of the glass substrate where the transparent electrode line was provided in a manner to cover the transparent electrode, thereby forming a 10-nm-thick hole injecting layer (also referred to as a third anode side organic layer). The ratios of the compound HT1 and the compound HA1 in the hole injecting layer were 90 mass % and 10 mass %, respectively.

The compound HT1 was vapor-deposited on the hole injecting layer to form an 80-nm-thick first hole transporting layer (also referred to as a second anode side organic layer).

A compound EB1 was vapor-deposited on the first hole transporting layer to form a 5-nm-thick second hole transporting layer (also referred to as an electron blocking layer or a first anode side organic layer).

A compound BH1-1 (first host material), a compound BH1-2 (second host material), and a compound BD1 (first luminescent compound) were co-deposited on the second hole transporting layer, thereby forming a 5-nm-thick first emitting layer. The ratios of the compound BH1-1, the compound BH1-2, and the compound BD1 in the first emitting layer were 74 mass %, 25 mass %, and 1 mass %, respectively.

A compound BH2-1 (third host material) and the compound BD1 (second luminescent compound) were co-deposited on the first emitting layer, thereby forming a 15-nm-thick second emitting layer. The ratios of the compound BH2-1 and the compound BD1 in the second emitting layer were 99 mass % and 1 mass %, respectively.

A compound HB1 was vapor-deposited on the second emitting layer to form a 5-nm-thick first electron transporting layer (also referred to as a hole blocking layer).

A compound ET1 and a compound Liq were co-deposited on the first electron transporting layer to form a 25-nm-thick second electron transporting layer. The ratios of the compound ET1 and the compound Liq in the second electron transporting layer were 50 mass % and 50 mass %, respectively. Liq is an abbreviation of (8-quinolinolato) lithium ((8-Quinolinolato) lithium).

Ytterbium (Yb) was vapor-deposited on the second electron transporting layer to form a 1-nm-thick electron injecting layer.

Metal A1 was vapor-deposited on the electron injecting layer to form an 80-nm-thick cathode.

A device arrangement of the organic EL device in Example 1 is roughly shown as follows.
ITO (130)/HT1:HA1 (10, 90%:10%)/HT1 (80)/EB1 (5)/
BH1-1:BH1-2:BD1 (5, 74%:25%:1%)/
BH2-1:BD1 (15, 99%:1%)/HB1 (5)/ET1:Liq (25, 50%:50%)/Yb (1)/Al(80)

Numerals in parentheses represent a film thickness (unit: nm).

In the device arrangement of Example 1, the numerals (90%: 10%) represented by percentage in the same parentheses indicate a ratio (mass %) between the compound HT1 and the compound HA1 in the hole injecting layer, the numerals (74%: 25%: 1%) represented by percentage in the same parentheses indicate a ratio (mass %) between the compound BH1-1, the compound BH1-2, and the compound BD1 in the first emitting layer, the numerals (99%: 1%) represented by percentage in the same parentheses indicate a ratio (mass %) between the compound BH2-1 and the compound BD1 in the second emitting layer, and the numerals (50%: 50%) represented by percentage in the same parentheses indicate a ratio (mass %) between the compound ET1 and the compound Liq in the second electron transporting layer. Similar notations apply to the description below.

### Example 2

An organic EL device in Example 2 was produced as in Example 1 except that the ratio between the compound BH1-1 and the compound BH1-2 in the first emitting layer was changed to that shown in Table 1.

### Comparative Example 1

An organic EL device in Comparative Example 1 was produced as in Example 1 except that the first emitting layer was formed using no compound BH1-2 and the first emitting layer was formed by co-deposition after changing the respective ratios of the compound BH1-1 and the compound BD1 to those shown in Table 1.

### Comparative Example 2

An organic EL device in Comparative Example 2 was produced as in Example 1 except that the first emitting layer was formed using no compound BH1-1 and the first emitting layer was formed by co-deposition after changing the respective ratios of the compound BH1-2 and the compound BD1 to those shown in Table 1.

### Evaluation (1) on Organic EL Device

The produced organic EL devices were evaluated as follows. Tables 1 to 8 show evaluation results. Tables 1 to 8 also show the singlet energy S₁, the triplet energy T₁, and HOMO of the compounds used for the emitting layers in each Example.

### External Quantum Efficiency EQE and Maximum Peak Wavelength λₚ

Voltage was applied to the organic EL device such that a current density was 10 mA/cm², where spectral radiance spectrum was measured by a spectroradiometer CS-2000 (produced by Konica Minolta, Inc.).

The external quantum efficiency EQE (unit: %) was calculated based on the obtained spectral radiance spectra, assuming that the spectra was provided under a Lambertian radiation.

The maximum peak wavelength λₚ (unit: nm) was calculated from the obtained spectral radiance spectrum.

### Lifetime LT95

Voltage was applied to the produced organic EL device so that a current density was 50 mA/cm², where a time (LT95 (unit: hr)) elapsed before a luminance intensity was reduced to 95% of the initial luminance intensity was measured as the lifetime. The luminance intensity was measured by using a spectroradiometer CS-2000 (produced by Konica Minolta, Inc.).

TABLE 1

| | | | Ex. 1 | Ex. 2 | Comp. 1 | Comp. 2 |
|---|---|---|---|---|---|---|
| First | First host | Name | BH1-1 | BH1-1 | BH1-1 | — |
| emitting | material | S₁ [eV] | 3.31 | 3.31 | 3.31 | — |
| layer | | T₁ [eV] | 2.09 | 2.09 | 2.09 | — |
| | | HOMO [eV] | −5.73 | −5.73 | −5.73 | — |
| | | Ratio | 74 | 49 | 99 | 0 |
| | | [mass %] | | | | |
| | Second host | Name | BH1-2 | BH1-2 | — | BH1-2 |
| | material | S₁ [eV] | 3.09 | 3.09 | — | 3.09 |
| | | T₁ [eV] | 2.08 | 2.08 | — | 2.08 |
| | | HOMO [eV] | −5.69 | −5.69 | — | −5.69 |
| | | Ratio | 25 | 50 | 0 | 99 |
| | | [mass %] | | | | |
| | First | Name | BD1 | BD1 | BD1 | BD1 |
| | luminescent | S₁[eV] | 2.78 | 2.78 | 2.78 | 2.78 |
| | compound | T₁[eV] | 2.32 | 2.32 | 2.32 | 2.32 |
| | | Ratio | 1 | 1 | 1 | 1 |
| | | [mass %] | | | | |
| Second | Third host | Name | BH2-1 | BH2-1 | BH2-1 | BH2-1 |
| emitting | material | S₁ [eV] | 3.00 | 3.00 | 3.00 | 3.00 |
| layer | | T₁ [eV] | 1.90 | 1.90 | 1.90 | 1.90 |
| | Second | Name | BD1 | BD1 | BD1 | BD1 |
| | luminescent | | | | | |
| | compound | | | | | |

| | | | | | |
|---|---|---|---|---|---|
| Evaluation | EQE [%] | 12.0 | 12.1 | 12.0 | 11.7 |
| results of | LT95 [hr] | 118 | 150 | 99 | 96 |
| device | λp [nm] | 451 | 451 | 451 | 451 |

### Example 3

An organic EL device in Example 3 was produced as in Example 1 except that the film thickness of each of the first and second emitting layers was changed to 10 nm.

### Example 4

An organic EL device in Example 4 was produced as in Example 2 except that the film thickness of each of the first and second emitting layers was changed to 10 nm.

### Comparative Example 3

An organic EL device in Comparative Example 3 was produced as in Comparative Example 1 except that the film thickness of each of the first and second emitting layers was changed to 10 nm.

### Comparative Example 4

An organic EL device in Comparative Example 4 was produced as in Comparative Example 2 except that the film thickness of each of the first and second emitting layers was changed to 10 nm.

TABLE 2

| | | | Ex. 3 | Ex. 4 | Comp. 3 | Comp. 4 |
|---|---|---|---|---|---|---|
| First | First host | Name | BH1-1 | BH1-1 | BH1-1 | — |
| emitting | material | S₁ [eV] | 3.31 | 3.31 | 3.31 | — |
| layer | | T₁ [eV] | 2.09 | 2.09 | 2.09 | — |
| | | HOMO [eV] | −5.73 | −5.73 | −5.73 | — |
| | | Ratio | 74 | 49 | 99 | 0 |
| | | [mass %] | | | | |
| | Second host | Name | BH1-2 | BH1-2 | — | BH1-2 |
| | material | S₁ [eV] | 3.09 | 3.09 | — | 3.09 |
| | | T₁ [eV] | 2.08 | 2.08 | — | 2.08 |
| | | HOMO [eV] | −5.69 | −5.69 | — | −5.69 |
| | | Ratio | 25 | 50 | 0 | 99 |
| | | [mass %] | | | | |
| | First | Name | BD1 | BD1 | BD1 | BD1 |
| | luminescent | S₁ [eV] | 2.78 | 2.78 | 2.78 | 2.78 |
| | compound | T₁ [eV] | 2.32 | 2.32 | 2.32 | 2.32 |
| | | Ratio | 1 | 1 | 1 | 1 |
| | | [mass %] | | | | |
| Second | Third host | Name | BH2-1 | BH2-1 | BH2-1 | BH2-1 |
| emitting | material | S₁ [eV] | 3.00 | 3.00 | 3.00 | 3.00 |
| layer | | T₁ [eV] | 1.90 | 1.90 | 1.90 | 1.90 |
| | Second | Name | BD1 | BD1 | BD1 | BD1 |
| | luminescent | | | | | |
| | compound | | | | | |

| | | | | | |
|---|---|---|---|---|---|
| Evaluation | EQE [%] | 12.0 | 11.9 | 11.8 | 11.5 |
| results of | LT95 [hr] | 120 | 115 | 108 | 80 |
| device | λp [nm] | 451 | 451 | 451 | 451 |

### Example 5

An organic EL device in Example 5 was produced as in Example 1 except that the film thickness of each of the first and second emitting layers was changed to 10 nm and a compound BH1-3 was used as the second host material.

### Example 6

An organic EL device in Example 6 was produced as in Example 2 except that the film thickness of each of the first and second emitting layers was changed to 10 nm and the compound BH1-3 was used as the second host material.

### Comparative Example 5

An organic EL device in Comparative Example 5 was produced as in Comparative Example 2 except that the film thickness of each of the first and second emitting layers was changed to 10 nm and the compound BH1-3 was used as the second host material.

Comparative Example 3 is shown again in Table 3 for comparison.

TABLE 3

| | | | Ex. 5 | Ex. 6 | Comp. 3 | Comp. 5 |
|---|---|---|---|---|---|---|
| First | First host | Name | BH1-1 | BH1-1 | BH1-1 | — |
| emitting | material | S₁ [eV] | 3.31 | 3.31 | 3.31 | — |
| layer | | T₁ [eV] | 2.09 | 2.09 | 2.09 | — |
| | | HOMO [eV] | −5.73 | −5.73 | −5.73 | — |
| | | Ratio | 74 | 49 | 99 | 0 |
| | | [mass %] | | | | |
| | Second host | Name | BH1-3 | BH1-3 | — | BH1-3 |
| | material | S₁ [eV] | 2.94 | 2.94 | — | 2.94 |
| | | T₁ [eV] | 2.20 | 2.20 | — | 2.20 |
| | | HOMO [eV] | −5.47 | −5.47 | — | −5.47 |
| | | Ratio | 25 | 50 | 0 | 99 |
| | | [mass %] | | | | |
| | First | Name | BD1 | BD1 | BD1 | BD1 |
| | luminescent | S₁ [eV] | 2.78 | 2.78 | 2.78 | 2.78 |
| | compound | T₁ [eV] | 2.32 | 2.32 | 2.32 | 2.32 |
| | | Ratio | 1 | 1 | 1 | 1 |
| | | [mass %] | | | | |
| Second | Third host | Name | BH2-1 | BH2-1 | BH2-1 | BH2-1 |
| emitting | material | S₁ [eV] | 3.00 | 3.00 | 3.00 | 3.00 |
| layer | | T₁ [eV] | 1.90 | 1.90 | 1.90 | 1.90 |
| | Second | Name | BD1 | BD1 | BD1 | BD1 |
| | luminescent | | | | | |
| | compound | | | | | |

| | | | | | |
|---|---|---|---|---|---|
| Evaluation | EQE [%] | 11.8 | 11.7 | 11.8 | 11.1 |
| results of | LT95 [hr] | 131 | 113 | 108 | 35 |
| device | λp [nm] | 451 | 451 | 451 | 451 |

### Example 7

An organic EL device in Example 7 was produced as in Example 1 except that the film thickness of each of the first and second emitting layers was changed to 10 nm and a compound BH1-4 was used as the second host material.

### Comparative Example 6

An organic EL device in Comparative Example 6 was produced as in Comparative Example 2 except that the film thickness of each of the first and second emitting layers was changed to 10 nm and the compound BH1-4 was used as the second host material.

TABLE 4

| | | | Ex. 7 | Comp. 3 | Comp. 6 |
|---|---|---|---|---|---|
| First | First host | Name | BH1-1 | BH1-1 | — |
| emitting | material | S₁ [eV] | 3.31 | 3.31 | — |
| layer | | T₁ [eV] | 2.09 | 2.09 | — |
| | | HOMO [eV] | −5.73 | −5.73 | — |
| | | Ratio [mass %] | 74 | 99 | 0 |
| | Second host | Name | BH1-4 | — | BH1-4 |
| | material | S₁ [eV] | 3.08 | — | 3.08 |
| | | T₁ [eV] | 2.22 | — | 2.22 |
| | | HOMO [eV] | −5.47 | — | −5.47 |
| | | Ratio [mass %] | 25 | 0 | 99 |
| | First | Name | BD1 | BD1 | BD1 |
| | luminescent | S₁ [eV] | 2.78 | 2.78 | 2.78 |
| | compound | T₁ [eV] | 2.32 | 2.32 | 2.32 |
| | | Ratio [mass %] | 1 | 1 | 1 |
| Second | Third host | Name | BH2-1 | BH2-1 | BH2-1 |
| emitting | material | S₁ [eV] | 3.00 | 3.00 | 3.00 |
| layer | | T₁ [eV] | 1.90 | 1.90 | 1.90 |
| | Second | Name | BD1 | BD1 | BD1 |
| | luminescent | | | | |
| | compound | | | | |

| | | | | |
|---|---|---|---|---|
| Evaluation | EQE [%] | 11.8 | 11.8 | 9.3 |
| results of | LT95 [hr] | 131 | 108 | 22 |
| device | Ap [nm] | 451 | 451 | 450 |

### Example 8

A glass substrate (size: 25 mm×75 mm×1.1 mm thick, produced by Geomatec Co., Ltd.) having an indium tin oxide (ITO) transparent electrode (anode) was ultrasonic-cleaned in isopropyl alcohol for five minutes, and then UV-ozone-cleaned for 30 minutes. The film thickness of the ITO transparent electrode was 130 nm.

After the glass substrate having the transparent electrode line was cleaned, the glass substrate was mounted on a substrate holder of a vacuum deposition apparatus. First, a compound HT2 and the compound HA1 were co-deposited on a surface of the glass substrate where the transparent electrode line was provided in a manner to cover the transparent electrode, thereby forming a 10-nm-thick hole injecting layer (also referred to as a third anode side organic layer). The ratios of the compound HT2 and the compound HA1 in the hole injecting layer were 97 mass % and 3 mass %, respectively.

The compound HT2 was vapor-deposited on the hole injecting layer to form an 85-nm-thick first hole transporting layer (also referred to as a second anode side organic layer).

The compound EB1 was vapor-deposited on the first hole transporting layer to form a 5-nm-thick second hole transporting layer (also referred to as an electron blocking layer or a first anode side organic layer).

A compound BH1-5 (first host material), a compound BH1-6 (second host material), and a compound BD2 (first luminescent compound) were co-deposited on the second hole transporting layer, thereby forming a 5-nm-thick first emitting layer. The ratios of the compound BH1-5, the compound BH1-6, and the compound BD2 in the first emitting layer were 79 mass %, 20 mass %, and 1 mass %, respectively.

A compound BH2-2 (third host material) and the compound BD2 (second luminescent compound) were co-deposited on the first emitting layer, thereby forming a 15-nm-thick second emitting layer. The ratios of the compound BH2-2 and the compound BD2 in the second emitting layer were 99 mass % and 1 mass %, respectively.

The compound HB1 was vapor-deposited on the second emitting layer to form a 5-nm-thick first electron transporting layer (also referred to as a hole blocking layer).

A compound ET2 and the compound Liq were co-deposited on the first electron transporting layer to form a 25-nm-thick second electron transporting layer. The ratios of the compound ET2 and the compound Liq in the second electron transporting layer were 50 mass % and 50 mass %, respectively.

The compound Liq was vapor-deposited on the second electron transporting layer to form a 1-nm-thick electron injecting layer.

Metal Al was vapor-deposited on the electron injecting layer to form an 80-nm-thick cathode.

A device arrangement of the organic EL device in Example 8 is roughly shown as follows.
ITO (130)/HT2:HA1 (10, 97%:3%)/HT1 (85)/EB1 (5)/
BH1-5:BH1-6:BD2 (5, 79%:20%:1%)/
BH2-2:BD2 (15, 99%:1%)/HB1 (5)/ET2:Liq (25, 50%:50%)/Liq (1)/Al(80)

### Comparative Example 7

An organic EL device in Comparative Example 7 was produced as in Example 8 except that the first emitting layer was formed using no compound BH1-6 and the first emitting layer was formed by co-deposition after changing the respective ratios of the compound BH1-5 and the compound BD2 to those shown in Table 5.

### Comparative Example 8

An organic EL device in Comparative Example 8 was produced as in Example 8 except that the first emitting layer was formed using no compound BH1-5 and the first emitting layer was formed by co-deposition after changing the respective ratios of the compound BH1-6 and the compound BD2 to those shown in Table 5.

TABLE 5

| | | | Ex. 8 | Comp. 7 | Comp. 8 |
|---|---|---|---|---|---|
| First | First host | Name | BH1-5 | BH1-5 | — |
| emitting | material | S₁ [eV] | 3.12 | 3.12 | — |
| layer | | T₁ [eV] | 2.09 | 2.09 | — |
| | | HOMO [eV] | −5.71 | −5.71 | — |
| | | Ratio [mass %] | 79 | 99 | 0 |
| | Second host | Name | BH1-6 | — | BH1-6 |
| | material | S₁ [eV] | 3.08 | — | 3.08 |
| | | T₁ [eV] | 2.09 | — | 2.09 |
| | | HOMO [eV] | −5.45 | — | −5.45 |
| | | Ratio [mass %] | 20 | 0 | 99 |
| | First | Name | BD2 | BD2 | BD2 |
| | luminescent | S₁ [eV] | 2.71 | 2.71 | 2.71 |
| | compound | T₁ [eV] | 2.64 | 2.64 | 2.64 |
| | | Ratio [mass %] | 1 | 1 | 1 |
| Second | Third host | Name | BH2-2 | BH2-2 | BH2-2 |
| emitting | material | S₁ [eV] | 3.01 | 3.01 | 3.01 |
| layer | | T₁ [eV] | 1.85 | 1.85 | 1.85 |
| | Second | Name | BD2 | BD2 | BD2 |
| | luminescent | | | | |
| | compound | | | | |

| | | | | |
|---|---|---|---|---|
| Evaluation | EQE [%] | 10.6 | 10.6 | 10.3 |
| results of | LT95 [hr] | 771 | 642 | 511 |
| device | Ap [nm] | 460 | 460 | 459 |

### Example 9

A glass substrate (size: 25 mm×75 mm×1.1 mm thick, produced by Geomatec Co., Ltd.) having an indium tin oxide (ITO) transparent electrode (anode) was ultrasonic-cleaned in isopropyl alcohol for five minutes, and then UV-ozone-cleaned for 30 minutes. The film thickness of the ITO transparent electrode was 130 nm.

After the glass substrate having the transparent electrode line was cleaned, the glass substrate was mounted on a substrate holder of a vacuum deposition apparatus. First, a compound HT3 and the compound HA1 were co-deposited on a surface of the glass substrate where the transparent electrode line was provided in a manner to cover the transparent electrode, thereby forming a 10-nm-thick hole injecting layer (also referred to as a third anode side organic layer). The ratios of the compound HT3 and the compound HA1 in the hole injecting layer were 97 mass % and 3 mass %, respectively.

The compound HT3 was vapor-deposited on the hole injecting layer to form a 75-nm-thick first hole transporting layer (also referred to as a second anode side organic layer).

A compound EB2 was vapor-deposited on the first hole transporting layer to form a 10-nm-thick second hole transporting layer (also referred to as an electron blocking layer or a first anode side organic layer).

The compound BH1-5 (first host material), the compound BH1-6 (second host material), and a compound BD3 (first luminescent compound) were co-deposited on the second hole transporting layer, thereby forming a 5-nm-thick first emitting layer. The ratios of the compound BH1-5, the compound BH1-6, and the compound BD3 in the first emitting layer were 48 mass %, 50 mass %, and 2 mass %, respectively.

A compound BH2-3 (third host material) and the compound BD3 (second luminescent compound) were co-deposited on the first emitting layer, thereby forming a 15-nm-thick second emitting layer. The ratios of the compound BH2-3 and the compound BD3 in the second emitting layer were 98 mass % and 2 mass %, respectively.

The compound HB1 was vapor-deposited on the second emitting layer to form a 5-nm-thick first electron transporting layer (also referred to as a hole blocking layer).

A compound ET3 and the compound Liq were co-deposited on the first electron transporting layer to form a 25-nm-thick second electron transporting layer. The ratios of the compound ET3 and the compound Liq in the second electron transporting layer were 50 mass % and 50 mass %, respectively.

Ytterbium (Yb) was vapor-deposited on the second electron transporting layer to form a 1-nm-thick electron injecting layer.

Metal Al was vapor-deposited on the electron injecting layer to form an 80-nm-thick cathode.

A device arrangement of the organic EL device in Example 9 is roughly shown as follows.
ITO (130)/HT3:HA1 (10, 97%:3%)/HT3 (75)/EB2 (10)/
BH1-5:BH1-6:BD3 (5, 48%:50%:2%)/
BH2-3:BD3 (15, 98%:2%)/HB1 (5)/ET3:Liq (25, 50%:50%)/Yb (1)/Al(80)

### Comparative Example 9

An organic EL device in Comparative Example 9 was produced as in Example 9 except that the first emitting layer was formed using no compound BH1-6 and the first emitting layer was formed by co-deposition after changing the respective ratios of the compound BH1-5 and the compound BD3 to those shown in Table 6.

### Comparative Example 10

An organic EL device in Comparative Example 10 was produced as in Example 9 except that the first emitting layer was formed using no compound BH1-5 and the first emitting layer was formed by co-deposition after changing the respective ratios of the compound BH1-6 and the compound BD3 to those shown in Table 6.

TABLE 6

| | | | Ex. 9 | Comp. 9 | Comp. 10 |
|---|---|---|---|---|---|
| First | First host | Name | BH1-5 | BH1-5 | — |
| emitting | material | S₁ [eV] | 3.12 | 3.12 | — |
| layer | | T₁ [eV] | 2.09 | 2.09 | — |
| | | HOMO [eV] | −5.71 | −5.71 | — |
| | | Ratio [mass %] | 48 | 98 | 0 |
| | Second host | Name | BH1-6 | — | BH1-6 |
| | material | S₁ [eV] | 3.08 | — | 3.08 |
| | | T₁ [eV] | 2.09 | — | 2.09 |
| | | HOMO [eV] | −5.45 | — | −5.45 |
| | | Ratio [mass %] | 50 | 0 | 98 |
| | First | Name | BD3 | BD3 | BD3 |
| | luminescent | S₁ [eV] | 2.73 | 2.73 | 2.73 |
| | compound | T₁ [eV] | 2.29 | 2.29 | 2.29 |
| | | Ratio [mass %] | 2 | 2 | 2 |
| Second | Third host | Name | BH2-3 | BH2-3 | BH2-3 |
| emitting | material | S₁ [eV] | 3.01 | 3.01 | 3.01 |
| layer | | T₁ [eV] | 1.86 | 1.86 | 1.86 |
| | Second | Name | BD3 | BD3 | BD3 |
| | luminescent | | | | |
| | compound | | | | |

| | | | | |
|---|---|---|---|---|
| Evaluation | EQE [%] | 12.1 | 12.0 | 11.7 |
| results of | LT95 [hr] | 321 | 283 | 228 |
| device | Ap [nm] | 459 | 460 | 459 |

### Example 10

An organic EL device in Example 10 was produced as in Example 9 except that the compound BH1-5 as the first host material was replaced with the compound BH1-1, and the ratios of the compound BH1-1, the compound BH1-6, and the compound BD3 in the first emitting layer were 49 mass %, 49 mass %, and 2 mass %, respectively.

### Example 11

An organic EL device in Example 11 was produced as in Example 9 except that the compound BH1-5 as the first host material was replaced with the compound BH1-1, and the ratios of the compound BH1-1, the compound BH1-6, and the compound BD3 in the first emitting layer were 24 mass %, 74 mass %, and 2 mass %, respectively.

### Example 12

An organic EL device in Example 12 was produced as in Example 9 except that the compound BH1-5 as the first host material was replaced with the compound BH1-1, and the ratios of the compound BH1-1, the compound BH1-6, and the compound BD3 in the first emitting layer were 10 mass %, 88 mass %, and 2 mass %, respectively.

### Comparative Example 11

An organic EL device in Comparative Example 11 was produced as in Example 10 except that the first emitting layer was formed using no compound BH1-6 and the first emitting layer was formed by co-deposition after changing the respective ratios of the compound BH1-1 and the compound BD3 to those shown in Table 7.

Comparative Example 10 is shown again in Table 7 for comparison.

TABLE 7

| | | | Ex. 10 | Ex. 11 | Ex. 12 | Comp. 11 | Comp. 10 |
|---|---|---|---|---|---|---|---|
| First | First host | Name | BH1-1 | BH1-1 | BH1-1 | BH1-1 | — |
| emitting | material | S₁ [eV] | 3.31 | 3.31 | 3.31 | 3.31 | — |
| layer | | T₁ [eV] | 2.09 | 2.09 | 2.09 | 2.09 | — |
| | | HOMO [eV] | −5.73 | −5.73 | −5.73 | −5.73 | — |
| | | Ratio | 49 | 24 | 10 | 98 | 0 |
| | | [mass %] | | | | | |
| | Second host | Name | BH1-6 | BH1-6 | BH1-6 | — | BH1-6 |
| | material | S₁ [eV] | 3.08 | 3.08 | 3.08 | — | 3.08 |
| | | T₁ [eV] | 2.09 | 2.09 | 2.09 | — | 2.09 |
| | | HOMO [eV] | −5.45 | −5.45 | −5.45 | — | −5.45 |
| | | Ratio | 49 | 74 | 88 | 0 | 98 |
| | | [mass %] | | | | | |
| | First | Name | BD3 | BD3 | BD3 | BD3 | BD3 |
| | luminescent | S₁ [eV] | 2.73 | 2.73 | 2.73 | 2.73 | 2.73 |
| | compound | T₁ [eV] | 2.29 | 2.29 | 2.29 | 2.29 | 2.29 |
| | | Ratio | 2 | 2 | 2 | 2 | 2 |
| | | [mass %] | | | | | |
| Second | Third host | Name | BH2-3 | BH2-3 | BH2-3 | BH2-3 | BH2-3 |
| emitting | material | S₁ [eV] | 3.01 | 3.01 | 3.01 | 3.01 | 3.01 |
| layer | | T₁ [eV] | 1.86 | 1.86 | 1.86 | 1.86 | 1.86 |
| | Second | Name | BD3 | BD3 | BD3 | BD3 | BD3 |
| | luminescent | | | | | | |
| | compound | | | | | | |

| | | | | | | |
|---|---|---|---|---|---|---|
| Evaluation | EQE [%] | 12.0 | 12.0 | 12.0 | 12.0 | 11.7 |
| results of | LT95 [hr] | 291 | 294 | 266 | 174 | 228 |
| device | λp [nm] | 460 | 460 | 460 | 460 | 459 |

### Example 13

An organic EL device in Example 13 was produced as in Example 9 except that the compound BH1-5 as the first host material was replaced with the compound BH1-2, and the ratios of the compound BH1-2, the compound BH1-6, and the compound BD3 in the first emitting layer were 48 mass %, 50 mass %, and 2 mass %, respectively.

### Comparative Example 12

An organic EL device in Comparative Example 12 was produced as in Example 13 except that the first emitting layer was formed using no compound BH1-6 and the first emitting layer was formed by co-deposition after changing the respective ratios of the compound BH1-2 and the compound BD3 to those shown in Table 8.

Comparative Example 10 is shown again in Table 8 for comparison.

TABLE 8

| | | | Ex. 13 | Comp. 12 | Comp. 10 |
|---|---|---|---|---|---|
| First | First host | Name | BH1-2 | BH1-2 | — |
| emitting | material | S₁ [eV] | 3.09 | 3.09 | — |
| layer | | T₁ [eV] | 2.08 | 2.08 | — |
| | | HOMO [eV] | −5.69 | −5.69 | — |
| | | Ratio [mass %] | 48 | 98 | 0 |
| | Second host | Name | BH1-6 | — | BH1-6 |
| | material | S₁ [eV] | 3.08 | — | 3.08 |
| | | T₁ [eV] | 2.09 | — | 2.09 |
| | | HOMO [eV] | −5.45 | — | −5.45 |
| | | Ratio [mass %] | 50 | 0 | 98 |
| | First | Name | BD3 | BD3 | BD3 |
| | luminescent | S₁ [eV] | 2.73 | 2.73 | 2.73 |
| | compound | T₁ [eV] | 2.29 | 2.29 | 2.29 |
| | | Ratio [mass %] | 2 | 2 | 2 |
| Second | Third host | Name | BH2-3 | BH2-3 | BH2-3 |
| emitting | material | S₁ [eV] | 3.01 | 3.01 | 3.01 |
| layer | | T₁ [eV] | 1.86 | 1.86 | 1.86 |
| | Second | Name | BD3 | BD3 | BD3 |
| | luminescent | | | | |
| | compound | | | | |

| | | | | |
|---|---|---|---|---|
| Evaluation | EQE [%] | 12.2 | 12.2 | 11.7 |
| results of | LT95 [hr] | 173 | 157 | 228 |
| device | Ap [nm] | 459 | 459 | 459 |

### Production (2) of Organic EL Device

The organic EL devices were produced as follows.

### Example 14

A glass substrate (size: 25 mm×75 mm×1.1 mm thick, produced by Geomatec Co., Ltd.) having an ITO transparent electrode (anode) was ultrasonic-cleaned in isopropyl alcohol for five minutes, and then UV-ozone-cleaned for 30 minutes. The film thickness of the ITO transparent electrode was 130 nm.

After the glass substrate having the transparent electrode line was cleaned, the glass substrate was mounted on a substrate holder of a vacuum deposition apparatus. First, a compound HT4 and the compound HA1 were co-deposited on a surface of the glass substrate where the transparent electrode line was provided in a manner to cover the transparent electrode, thereby forming a 10-nm-thick hole injecting layer (also referred to as a third anode side organic layer). The ratios of the compound HT4 and the compound HA1 in the hole injecting layer were 97 mass % and 3 mass %, respectively.

The compound HT4 was vapor-deposited on the hole injecting layer to form an 85-nm-thick first hole transporting layer (also referred to as a second anode side organic layer).

The compound EB1 was vapor-deposited on the first hole transporting layer to form a 5-nm-thick second hole transporting layer (also referred to as an electron blocking layer or a first anode side organic layer).

A compound BH1-7 (first host material), a compound BH1-10 (second host material), and a compound BD4 (first luminescent compound) were co-deposited on the second hole transporting layer, thereby forming a 5-nm-thick first emitting layer. The ratios of the compound BH1-7, the compound BH1-10, and the compound BD4 in the first emitting layer were 89 mass %, 10 mass %, and 1 mass %, respectively.

A compound BH2-4 (third host material) and the compound BD4 (second luminescent compound) were co-deposited on the first emitting layer, thereby forming a 15-nm-thick second emitting layer. The ratios of the compound BH2-4 and the compound BD4 in the second emitting layer were 99 mass % and 1 mass %, respectively.

A compound HB2 was vapor-deposited on the second emitting layer to form a 5-nm-thick first electron transporting layer (also referred to as a hole blocking layer).

A compound ET4 and the compound Liq were co-deposited on the first electron transporting layer to form a 31-nm-thick second electron transporting layer. The ratios of the compound ET4 and the compound Liq in the second electron transporting layer were 50 mass % and 50 mass %, respectively.

The compound Liq was vapor-deposited on the second electron transporting layer to form a 1-nm-thick electron injecting layer.

Metal Al was vapor-deposited on the electron injecting layer to form an 80-nm-thick cathode.

A device arrangement of the organic EL device in Example 14 is roughly shown as follows.
ITO (130)/HT4:HA1 (10, 97%:3%)/HT4 (85)/EB1 (5)/
BH1-7:BH1-10:BD4 (5, 89%:10%: 1%)/
BH2-4:BD4 (15, 99%:1%)/HB2 (5)/ET4:Liq (31, 50%:50%)/Liq (1)/Al(80)

### Examples 15 to 31

Organic EL devices in Examples 15 to 31 were each produced as in Example 14 except that at least one of the first host material, the second host material, the third host material, or the ratio of the compound in the first emitting layer was changed as shown in Tables 9 to 12.

### Comparative Examples 13 to 21

Organic EL devices in Comparative Examples 13 to 21 were each produced as in Example 14 except that the first emitting layer was formed using no second host material and the first emitting layer was formed by co-deposition after changing the ratios of the first host material and the first luminescent compound to those shown in Tables 9 to 11.

### Comparative Examples 22 to 24

Organic EL devices in Comparative Examples 22 to 24 were each produced as in Example 14 except that the first emitting layer was formed using no first host material and the first emitting layer was formed by co-deposition after changing the ratios of the second host material and the first luminescent compound to those shown in Table 12.

### Evaluation (2) on Organic EL Devices

The produced organic EL devices were evaluated as follows. Tables 9 to 12 show evaluation results. Tables 9 to 12 also show the singlet energy S₁, the triplet energy T₁, and HOMO of the compounds used for the emitting layers in each Example.

### External Quantum Efficiency EQE and Maximum Peak Wavelength λₚ

The external quantum efficiency EQE and the maximum peak wavelength λₚ were measured similarly as the method described in Evaluation (1) on Organic EL Devices above.

### Lifetime LT90

Voltage was applied to the produced organic EL device so that a current density was 50 mA/cm², where a time (LT90 (unit: hr)) elapsed before a luminance intensity was reduced to 90% of the initial luminance intensity was measured as the lifetime. The luminance intensity was measured by using a spectroradiometer CS-2000 (produced by Konica Minolta, Inc.).

TABLE 9

| | | | Ex. 14 | Ex. 15 | Ex. 16 | Ex. 17 | Comp. 13 | Ex. 18 | Comp. 14 | Ex. 19 | Comp. 15 |
|---|---|---|---|---|---|---|---|---|---|---|---|
| First | First host | Name | BH1-7 | BH1-7 | BH1-7 | BH1-7 | BH1-7 | BH1-7 | BH1-7 | BH1-7 | BH1-7 |
| emitting | material | S₁ [eV] | 3.08 | 3.08 | 3.08 | 3.08 | 3.08 | 3.08 | 3.08 | 3.08 | 3.08 |
| layer | | T₁ [eV] | 2.09 | 2.09 | 2.09 | 2.09 | 2.09 | 2.09 | 2.09 | 2.09 | 2.09 |
| | | HOMO [eV] | −5.79 | −5.79 | −5.79 | −5.79 | −5.79 | −5.79 | −5.79 | −5.79 | −5.79 |
| | | Ratio | 89 | 79 | 64 | 79 | 99 | 40 | 99 | 79 | 99 |
| | | [mass %] | | | | | | | | | |
| | Second host | Name | BH1-10 | BH1-12 | BH1-13 | BH1-14 | — | BH1-10 | — | BH1-10 | — |
| | material | S₁ [eV] | 3.15 | 3.03 | 2.99 | 3.20 | — | 3.15 | — | 3.15 | — |
| | | T₁ [eV] | 2.10 | 2.09 | 2.09 | 2.09 | — | 2.10 | — | 2.10 | — |
| | | HOMO [eV] | −5.66 | −5.51 | −5.50 | −5.45 | — | −5.66 | — | −5.66 | — |
| | | Ratio | 10 | 20 | 35 | 20 | — | 59 | — | 20 | — |
| | | [mass %] | | | | | | | | | |
| | First | Name | BD4 | BD4 | BD4 | BD4 | BD4 | BD4 | BD4 | BD4 | BD4 |
| | luminescent | S₁ [eV] | 2.80 | 2.80 | 2.80 | 2.80 | 2.80 | 2.80 | 2.80 | 2.80 | 2.80 |
| | compound | T₁ [eV] | 2.44 | 2.44 | 2.44 | 2.44 | 2.44 | 2.44 | 2.44 | 2.44 | 2.44 |
| | | Ratio | 1 | 1 | 1 | 1 | 1 | 1 | 1 | 1 | 1 |
| | | [mass %] | | | | | | | | | |
| Second | Third host | Name | BH2-4 | BH2-4 | BH2-4 | BH2-4 | BH2-4 | BH2-5 | BH2-5 | BH2-6 | BH2-6 |
| emitting | material | S₁ [eV] | 3.01 | 3.01 | 3.01 | 3.01 | 3.01 | 3.03 | 3.03 | 3.01 | 3.01 |
| layer | | T₁ [eV] | 1.81 | 1.81 | 1.81 | 1.81 | 1.81 | 1.81 | 1.81 | 1.86 | 1.86 |
| | Second | Name | BD4 | BD4 | BD4 | BD4 | BD4 | BD4 | BD4 | BD4 | BD4 |
| | luminescent | | | | | | | | | | |
| | compound | | | | | | | | | | |

| | | | | | | | | | | |
|---|---|---|---|---|---|---|---|---|---|---|
| Evaluation | EQE [%] | 9.22 | 9.69 | 9.21 | 9.21 | 9.16 | 10.90 | 10.90 | 9.46 | 9.46 |
| results of | LT95 [hr] | 258 | 400 | 454 | 498 | 182 | 399 | 379 | 213 | 151 |
| device | λp [nm] | 462 | 461 | 462 | 462 | 462 | 461 | 461 | 462 | 462 |

TABLE 10

| | | | Ex. 20 | Ex. 21 | Ex. 22 | Ex. 23 | Comp. 16 | Ex. 24 | Comp. 17 | Ex. 25 | Comp. 18 |
|---|---|---|---|---|---|---|---|---|---|---|---|
| First | First host | Name | BH1-8 | BH1-8 | BH1-8 | BH1-8 | BH1-8 | BH1-8 | BH1-8 | BH1-8 | BH1-8 |
| emitting | material | S₁ [eV] | 3.19 | 3.19 | 3.19 | 3.19 | 3.19 | 3.19 | 3.19 | 3.19 | 3.19 |
| layer | | T₁ [eV] | 2.07 | 2.07 | 2.07 | 2.07 | 2.07 | 2.07 | 2.07 | 2.07 | 2.07 |
| | | HOMO [eV] | −5.80 | −5.80 | −5.80 | −5.80 | −5.80 | −5.80 | −5.80 | −5.80 | −5.80 |
| | | Ratio | 89 | 79 | 64 | 79 | 99 | 49.5 | 99 | 49.5 | 99 |
| | | [mass %] | | | | | | | | | |
| | Second host | Name | BH1-10 | BH1-12 | BH1-13 | BH1-14 | — | BH1-10 | — | BH1-1 | — |
| | material | S₁ [eV] | 3.15 | 3.03 | 2.99 | 3.20 | — | 3.15 | — | 3.15 | — |
| | | T₁ [eV] | 2.10 | 2.09 | 2.09 | 2.09 | — | 2.10 | — | 2.10 | — |
| | | HOMO [eV] | −5.66 | −5.51 | −5.50 | −5.45 | — | −5.66 | — | −5.66 | — |
| | | Ratio | 10 | 20 | 35 | 20 | — | 49.5 | — | 49.5 | — |
| | | [mass %] | | | | | | | | | |
| | First | Name | BD4 | BD4 | BD4 | BD4 | BD4 | BD4 | BD4 | BD4 | BD4 |
| | luminescent | S₁ [eV] | 2.80 | 2.80 | 2.80 | 2.80 | 2.80 | 2.80 | 2.80 | 2.80 | 2.80 |
| | compound | T₁ [eV] | 2.44 | 2.44 | 2.44 | 2.44 | 2.44 | 2.44 | 2.44 | 2.44 | 2.44 |
| | | Ratio | 1 | 1 | 1 | 1 | 1 | 1 | 1 | 1 | 1 |
| | | [mass %] | | | | | | | | | |
| Second | Third host | Name | BH2-4 | BH2-4 | BH2-4 | BH2-4 | BH2-4 | BH2-5 | BH2-5 | BH2-6 | BH2-6 |
| emitting | material | S₁ [eV] | 3.01 | 3.01 | 3.01 | 3.01 | 3.01 | 3.03 | 3.03 | 3.01 | 3.01 |
| layer | | T₁ [eV] | 1.81 | 1.81 | 1.81 | 1.81 | 1.81 | 1.81 | 1.81 | 1.86 | 1.86 |
| | Second | Name | BD4 | BD4 | BD4 | BD4 | BD4 | BD4 | BD4 | BD4 | BD4 |
| | luminescent | | | | | | | | | | |
| | compound | | | | | | | | | | |

| | | | | | | | | | | |
|---|---|---|---|---|---|---|---|---|---|---|
| Evaluation | EQE [%] | 9.45 | 9.54 | 9.50 | 9.44 | 9.49 | 10.92 | 10.88 | 9.84 | 9.82 |
| results of | LT95 [hr.] | 94 | 149 | 202 | 194 | 54 | 185 | 90 | 93 | 50 |
| device | λp [nm] | 461 | 461 | 461 | 461 | 461 | 461 | 460 | 461 | 460 |

TABLE 11

| | | | Ex. 26 | Comp. 19 | Ex. 27 | Comp. 20 | Ex. 28 | Comp. 21 |
|---|---|---|---|---|---|---|---|---|
| First | First host | Name | BH1-9 | BH1-9 | BH1-9 | BH1-9 | BH1-9 | BH1-9 |
| emitting | material | S₁ [eV] | 3.10 | 3.10 | 3.10 | 3.10 | 3.10 | 3.10 |
| layer | | T₁ [eV] | 2.07 | 2.07 | 2.07 | 2.07 | 2.07 | 2.07 |
| | | HOMO [eV] | −5.83 | −5.83 | −5.83 | −5.83 | −5.83 | −5.83 |
| | | Ratio | 79 | 99 | 49.5 | 99 | 49.5 | 99 |
| | | [mass %] | | | | | | |
| | Second host | Name | BH1-10 | — | BH1-10 | — | BH1-10 | — |
| | material | S₁ [eV] | 3.15 | — | 3.15 | — | 3.15 | — |
| | | T₁ [eV] | 2.10 | — | 2.10 | — | 2.10 | — |
| | | HOMO [eV] | −5.66 | — | −5.66 | — | −5.66 | — |
| | | Ratio | 20 | — | 49.5 | — | 49.5 | — |
| | | [mass %] | | | | | | |
| | First | Name | BD4 | BD4 | BD4 | BD4 | BD4 | BD4 |
| | luminescent | S₁ [eV] | 2.80 | 2.80 | 2.80 | 2.80 | 2.80 | 2.80 |
| | compound | T₁ [eV] | 2.44 | 2.44 | 2.44 | 2.44 | 2.44 | 2.44 |
| | | Ratio | 1 | 1 | 1 | 1 | 1 | 1 |
| | | [mass %] | | | | | | |
| Second | Third host | Name | BH2-4 | BH2-4 | BH2-5 | BH2-5 | BH2-6 | BH2-6 |
| emitting | material | S₁ [eV] | 3.01 | 3.01 | 3.03 | 3.03 | 3.01 | 3.01 |
| layer | | T₁ [eV] | 1.81 | 1.81 | 1.81 | 1.81 | 1.86 | 1.86 |
| | Second | Name | BD4 | BD4 | BD4 | BD4 | BD4 | BD4 |
| | luminescent | | | | | | | |
| | compound | | | | | | | |

| | | | | | | | |
|---|---|---|---|---|---|---|---|
| Evaluation | EQE [%] | 9.05 | 9.07 | 10.56 | 10.54 | 9.74 | 9.70 |
| results of | LT95 [hr] | 113 | 61 | 205 | 122 | 100 | 60 |
| device | λp [nm] | 462 | 46 | 461 | 460 | 462 | 462 |

TABLE 12

| | | | Ex. 29 | Comp. 22 | Ex. 30 | Comp. 23 | Ex. 31 | Comp. 24 |
|---|---|---|---|---|---|---|---|---|
| First | First host | Name | BH1-10 | — | BH1-10 | — | BH1-10 | — |
| emitting | material | S₁ [eV] | 3.15 | — | 3.15 | — | 3.15 | — |
| layer | | T₁ [eV] | 2.10 | — | 2.10 | — | 2.10 | — |
| | | HOMO [eV] | −5.66 | — | −5.66 | — | −5.66 | — |
| | | Ratio | 10 | — | 59 | — | 10 | — |
| | | [mass %] | | | | | | |
| | Second host | Name | BH1-11 | BH1-11 | BH1-11 | BH1-11 | BH1-11 | BH1-11 |
| | material | S₁ [eV] | 3.08 | 3.08 | 3.08 | 3.08 | 3.08 | 3.08 |
| | | T₁ [eV] | 2.07 | 2.07 | 2.07 | 2.07 | 2.07 | 2.07 |
| | | HOMO [eV] | −5.50 | −5.50 | −5.50 | −5.50 | −5.50 | −5.50 |
| | | Ratio | 89 | 99 | 40 | 99 | 89 | 99 |
| | | [mass %] | | | | | | |
| | First | Name | BD4 | BD4 | BD4 | BD4 | BD4 | BD4 |
| | luminescent | S₁ [eV] | 2.80 | 2.80 | 2.80 | 2.80 | 2.80 | 2.80 |
| | compound | T₁ [eV] | 2.44 | 2.44 | 2.44 | 2.44 | 2.44 | 2.44 |
| | | Ratio | 1 | 1 | 1 | 1 | 1 | 1 |
| | | [mass %] | | | | | | |
| Second | Third host | Name | BH2-4 | BH2-4 | BH2-5 | BH2-5 | BH2-6 | BH2-6 |
| emitting | material | S₁ [eV] | 3.01 | 3.01 | 3.03 | 3.03 | 3.01 | 3.01 |
| layer | | T₁ [eV] | 1.81 | 1.81 | 1.81 | 1.81 | 1.86 | 1.86 |
| | Second | Name | BD4 | BD4 | BD4 | BD4 | BD4 | BD4 |
| | luminescent | | | | | | | |
| | compound | | | | | | | |

| | | | | | | | |
|---|---|---|---|---|---|---|---|
| Evaluation | EQE [%] | 9.73 | 9.73 | 11.01 | 11.04 | 10.25 | 10.26 |
| results of | LT95 [hr] | 378 | 317 | 482 | 369 | 345 | 313 |
| device | λp [nm] | 462 | 462 | 462 | 462 | 462 | 462 |

### Evaluation on Compounds

Compounds used for producing the organic EL devices in Examples and Comparative Examples were evaluated by methods below.

### Energy Level of Highest Occupied Molecular Orbital HOMO

A value of the energy level of the highest occupied molecular orbital HOMO of a measurement target was calculated by a numerical formula (Numerical Formula F3) below. The unit of the energy level of the highest occupied molecular orbital HOMO is eV.

$$HOMO = - 1.4 \times ( Eox - Efc ) - 4.6 eV \qquad ( F 3 )$$

In the numerical formula (Numerical Formula F3), Eox and Efc are as follows:
Eox: first oxidation potential (DPV, positive scan)
Efc: first oxidation potential of ferrocene (DPV, positive scan), (ca. +0.55 V vs Ag/AgCl)

Oxidation-reduction potential was measured by the differential pulse voltammetry (DPV) method using an electrochemical analyzer (CHI852D produced by ALS).

A sample solution used for the measurement was prepared by dissolving the measurement target and a supporting electrolyte in a solvent. In the sample solution, the concentrations of the measurement target and the supporting electrolyte were 1.0 mmol/L and 100 mmol/L, respectively. N, N-dimethylformamide (DMF) was used as the solvent. Tetrabutylammmonium hexafluorophosphate (TBHP) was used as the supporting electrolyte. A glassy carbon electrode was used as a working electrode. A platinum electrode was used as a counter electrode.

### Triplet Energy T₁

A measurement target compound was dissolved in EPA (diethylether:isopentane:ethanol=5:5:2 in volume ratio) at a concentration of 10 μmol/L, and the obtained solution was put in a quartz cell to provide a measurement sample. A phosphorescence spectrum (ordinate axis: phosphorescent luminous intensity, abscissa axis: wavelength) of the measurement sample was measured at a low temperature (77K). A tangent was drawn to the rise of the phosphorescence spectrum close to the short-wavelength region. An energy amount was calculated by a conversion equation (F1) below on a basis of a wavelength value λ_{edge} [nm] at an intersection of the tangent and the abscissa axis. The calculated energy amount was defined as triplet energy T₁. It should be noted that the triplet energy T₁ may have an error of about plus or minus 0.02 eV depending on measurement conditions.

$$Conversion Equation ( F 1 ) : {T}_{1} [ eV ] = 1239 \text{.85} / λ edge$$

The tangent to the rise of the phosphorescence spectrum close to the short-wavelength region is drawn as follows. While moving on a curve of the phosphorescence spectrum from the short-wavelength region to the local maximum value closest to the short-wavelength region among the local maximum values of the phosphorescence spectrum, a tangent is checked at each point on the curve toward the long-wavelength region of the phosphorescence spectrum. An inclination of the tangent is increased along the rise of the curve (i.e., a value of the ordinate axis is increased). A tangent drawn at a point of the local maximum inclination (i.e., a tangent at an inflection point) is defined as the tangent to the rise of the phosphorescence spectrum close to the short-wavelength region.

A local maximum point where a peak intensity is 15% or less of the maximum peak intensity of the spectrum is not counted as the above-mentioned local maximum peak intensity closest to the short-wavelength region. The tangent drawn at a point that is closest to the local maximum peak intensity closest to the short-wavelength region and where the inclination of the curve is the local maximum is defined as a tangent to the rise of the phosphorescence spectrum close to the short-wavelength region.

For phosphorescence measurement, a spectrophotofluorometer body F-4500 produced by Hitachi High-Technologies Corporation was used.

### Singlet Energy S₁

A toluene solution of a measurement target compound at a concentration of 10 μmol/L was prepared and put in a quartz cell. An absorption spectrum (ordinate axis: absorption intensity, abscissa axis: wavelength) of the thus-obtained sample was measured at a normal temperature (300K). A tangent was drawn to the fall of the absorption spectrum close to the long-wavelength region, and a wavelength value λ_{edge} [nm] at an intersection of the tangent and the abscissa axis was assigned to a conversion equation (F2) below to calculate a singlet energy.

$$Conversion Equation ( F 2 ) : {S}_{1} [ eV ] = 1239 \text{.85} / λ edge$$

A spectrophotometer (U3310 produced by Hitachi, Ltd.) was used for measuring the absorption spectrum.

The tangent to the fall of the absorption spectrum close to the long-wavelength region is drawn as follows. While moving on a curve of the absorption spectrum from the local maximum value closest to the long-wavelength region, among the local maximum values of the absorption spectrum, in a long-wavelength direction, a tangent at each point on the curve is checked. An inclination of the tangent is decreased and increased in a repeated manner as the curve falls (i.e., a value of the ordinate axis is decreased). A tangent drawn at a point where the inclination of the curve is the local minimum closest to the long-wavelength region (except when absorbance is 0.1 or less) is defined as the tangent to the fall of the absorption spectrum close to the long-wavelength region.

The local maximum absorbance of 0.2 or less is not counted as the above-mentioned local maximum absorbance closest to the long-wavelength region.

### Measurement of Maximum Fluorescence Peak Wavelength (FL-Peak)

A measurement target compound was dissolved in toluene at a concentration of 4.9×10⁻⁶ mol/L to prepare a toluene solution. Using a fluorescence spectrometer (spectrophotofluorometer F-7000 manufactured by Hitachi High-Tech Science Corporation), the toluene solution of the measurement target compound was excited at 390 nm, where a maximum fluorescence peak wavelength λ (unit: nm) was measured.

The maximum fluorescence peak wavelength A of the compound BD1 was 445 nm.

The maximum fluorescence peak wavelength λ of the compound BD2 was 455 nm.

The maximum fluorescence peak wavelength λ of the compound BD3 was 452 nm.

The maximum fluorescence peak wavelength λ of the compound BD4 was 457 nm.

Table 13 shows evaluation results on compounds.

TABLE 13

| | S₁ | T₁ | HOMO |
|---|---|---|---|
| Name | [eV] | [eV] | [eV] |
| EB1 | — | — | −5.29 |
| EB2 | — | — | −5.30 |
| BH1-1 | 3.31 | 2.09 | −5.73 |
| BH1-2 | 3.09 | 2.08 | −5.69 |
| BH1-3 | 2.94 | 2.20 | −5.47 |
| BH1-4 | 3.08 | 2.22 | −5.47 |
| BH1-5 | 3.12 | 2.09 | −5.71 |
| BH1-6 | 3.08 | 2.09 | −5.45 |
| BH1-7 | 3.08 | 2.09 | −5.79 |
| BH1-8 | 3.19 | 2.07 | −5.80 |
| BH1-9 | 3.10 | 2.07 | −5.83 |
| BH1-10 | 3.15 | 2.10 | −5.66 |
| BH1-11 | 3.08 | 2.07 | −5.50 |
| BH1-12 | 3.03 | 2.09 | −5.51 |
| BH1-13 | 2.99 | 2.09 | −5.50 |
| BH1-14 | 3.20 | 2.09 | −5.45 |
| BH2-1 | 3.00 | 1.90 | −5.75 |
| BH2-2 | 3.01 | 1.85 | −5.82 |
| BH2-3 | 3.01 | 1.86 | −5.78 |
| BH2-4 | 3.01 | 1.81 | −5.86 |
| BH2-5 | 3.03 | 1.81 | −5.78 |
| BH2-6 | 3.01 | 1.86 | −5.78 |
| BD1 | 2.78 | 2.32 | — |
| BD2 | 2.71 | 2.64 | — |
| BD3 | 2.73 | 2.29 | — |
| BD4 | 2.80 | 2.44 | — |

### EXPLANATION OF CODES

1 . . . organic EL device, 1A . . . organic EL device, 10 . . . organic layer, 10A . . . organic layer, 2 . . . substrate, 3 . . . anode, 4 . . . cathode, 5 . . . emitting region, 5A . . . emitting region, 51 . . . first emitting layer, 52 . . . second emitting layer, 6 . . . first hole transporting zone, 61 . . . first anode side organic layer, 62 . . . second anode side organic layer, 63 . . . third anode side organic layer, 7 . . . first electron transporting zone, 71 . . . electron transporting layer, 72 . . . electron injecting layer.

1. An organic electroluminescence device, comprising:
an anode;
a cathode;
an emitting region disposed between the anode and the cathode; and
a first anode side organic layer disposed between the emitting region and the anode, wherein
the first anode side organic layer comprises a first material,
the emitting region comprises a first emitting layer and a second emitting layer,
the first emitting layer is disposed between the first anode side organic layer and the second emitting layer,
the first emitting layer is in direct contact with the first anode side organic layer,
the first emitting layer comprises a first host material and a second host material,
the second emitting layer comprises a third host material,
an energy level of a highest occupied molecular orbital of the first material HOMO(HT1), an energy level of a highest occupied molecular orbital of the first host material HOMO(H1), and an energy level of a highest occupied molecular orbital of the second host material HOMO(H2) satisfy a relationship of a numerical formula (Numerical Formula A1) below,
a triplet energy of the first host material T₁(H1) and a triplet energy of the third host material T₁(H3) satisfy a relationship of a numerical formula (Numerical Formula A2) below, and
a triplet energy of the second host material T₁(H2) and a triplet energy of the third host material T₁(H3) satisfy a relationship of a numerical formula (Numerical Formula A3) below,
$$HOMO ( HT 1 ) > HOMO ( H 2 ) > HOMO ( H 1 ) \qquad ( Numerical Formula A 1 )$$
$${T}_{1} ( H 1 ) > {T}_{1} ( H 3 ) \qquad ( Numerical Formula A 2 )$$
$${T}_{1} ( H 2 ) > {T}_{1} ( H 3 ) . \qquad ( Numerical Formula A 3 )$$

2. The organic electroluminescence device according to claim 1, wherein
the triplet energy of the first host material T₁(H1) satisfies a relationship of a numerical formula (Numerical Formula A4) below, and
the triplet energy of the second host material T₁(H2) satisfies a relationship of a numerical formula (Numerical Formula A5) below,
$$1.8 eV < {T}_{1} ( H 1 ) < 2.5 eV \qquad ( Numerical Formula A 4 )$$
$$1.8 eV < {T}_{1} ( H 2 ) < 2.5 eV . \qquad ( Numerical Formula A 5 )$$

3. The organic electroluminescence device according to claim 1, wherein
the triplet energy of the first host material T₁(H1) satisfies a relationship of a numerical formula (Numerical Formula A41) below, and
the triplet energy of the second host material T₁(H2) satisfies a relationship of a numerical formula (Numerical Formula A51) below,
$$2. eV < {T}_{1} ( H 1 ) < 2.3 eV \qquad ( Numerical Formula A 41 )$$
$$2. eV < {T}_{1} ( H 2 ) < 2.3 eV . \qquad ( Numerical Formula A 51 )$$

4. The organic electroluminescence device according to claim 1, wherein HOMO(H2) above satisfies a relationship of a numerical formula (Numerical Formula A10) below,
$$HOMO ( H 2 ) > - 5.7 eV . \qquad ( Numerical Formula A10 )$$

5. The organic electroluminescence device according to claim 1, wherein HOMO(H1) above satisfies a relationship of a numerical formula (Numerical Formula A11) below,
$$- 5.6 eV > HOMO ( H 1 ) . \qquad ( Numerical formula A11 )$$

6. The organic electroluminescence device according to claim 1, wherein
the first emitting layer comprises a first luminescent compound, and
the second emitting layer comprises a second luminescent compound.

7. The organic electroluminescence device according to claim 6, wherein the first luminescent compound and the second luminescent compound are each independently a compound that emits light having a maximum peak wavelength of 500 nm or less.

8. An organic electroluminescence device, comprising:
an anode;
a cathode; and
an emitting region disposed between the anode and the cathode, wherein
the emitting region comprises a first emitting layer,
the first emitting layer comprises a first host material and a second host material,
the first host material is different from the second host material,
a triplet energy of the first host material T₁(H1) satisfies a relationship of a numerical formula (Numerical Formula A4) below, and
a triplet energy of the second host material T₁(H2) satisfies a relationship of a numerical formula (Numerical Formula A5) below,
$$1.8 eV < {T}_{1} ( H 1 ) < 2.5 eV \qquad ( Numerical Formula A 4 )$$
$$1.8 eV < {T}_{1} ( H 2 ) < 2.5 eV . \qquad ( Numerical Formula A 5 )$$

9. The organic electroluminescence device according to claim 8, wherein
the triplet energy of the first host material T₁(H1) satisfies a relationship of a numerical formula (Numerical Formula A41) below, and
the triplet energy of the second host material T₁(H2) satisfies a relationship of a numerical formula (Numerical Formula A51) below,
$$2. eV < {T}_{1} ( H 1 ) < 2.3 eV \qquad ( Numerical Formula A 41 )$$
$$2. eV < {T}_{1} ( H 2 ) < 2.3 eV . \qquad ( Numerical Formula A 51 )$$

10. The organic electroluminescence device according to claim 8, wherein an energy level of a highest occupied molecular orbital of the second host material HOMO(H2) above satisfies a relationship of a numerical formula (Numerical Formula A10) below,
$$HOMO ( H 2 ) > - 5.7 eV . \qquad ( Numerical Formula A10 )$$

11. The organic electroluminescence device according to claim 8, wherein an energy level of a highest occupied molecular orbital of the first host material HOMO(H1) above satisfies a relationship of a numerical formula (Numerical Formula A11) below,
$$- 5.6 eV > HOMO ( H 1 ) . \qquad ( Numerical formula A11 )$$

12. The organic electroluminescence device according to claim 8, further comprising a first anode side organic layer disposed between the emitting region and the anode, wherein
the first emitting layer is in direct contact with the first anode side organic layer,
the first anode side organic layer comprises a first material,
the first material, the first host material, and the second host material are mutually different, and
an energy level of a highest occupied molecular orbital of the first material HOMO(HT1), an energy level of a highest occupied molecular orbital of the first host material HOMO(H1), and an energy level of a highest occupied molecular orbital of the second host material HOMO(H2) satisfy a relationship of a numerical formula (Numerical Formula A1) below,
$$HOMO ( HT 1 ) > HOMO ( H 2 ) > HOMO ( H 1 ) . \qquad ( Numerical Formula A 1 )$$

13. The organic electroluminescence device according to claim 8, wherein the first emitting layer comprises a first luminescent compound.

14. The organic electroluminescence device according to claim 13, wherein the first luminescent compound is a compound that emits light having a maximum peak wavelength of 500 nm or less.

15. The organic electroluminescence device according to claim 8, wherein
the emitting region comprises a second emitting layer,
the second emitting layer comprises a third host material,
the first host material, the second host material, and the third host material are mutually different,
the triplet energy of the first host material T₁(H1) and a triplet energy of the third host material T₁(H3) satisfy a relationship of a numerical formula (Numerical Formula A2) below, and
the triplet energy of the second host material T₁(H2) and the triplet energy of the third host material T₁(H3) satisfy a relationship of a numerical formula (Numerical Formula A3) below,
$${T}_{1} ( H 1 ) > {T}_{1} ( H 3 ) \qquad ( Numerical Formula A 2 )$$
$${T}_{1} ( H 2 ) > {T}_{1} ( H 3 ) . \qquad ( Numerical Formula A 3 )$$

16. The organic electroluminescence device according to claim 15, wherein the second emitting layer comprises a second luminescent compound.

17. The organic electroluminescence device according to claim 16, wherein the second luminescent compound is a compound that emits light having a maximum peak wavelength of 500 nm or less.

18. A composition comprising a first compound and a second compound, wherein
the first compound and the second compound are mutually different compounds, and
the first compound and the second compound each independently comprise, in a molecule, at least one of a structure of Condition (i) or a structure of Condition (ii) below,
Condition (i): a biphenyl structure including a first benzene ring and a second benzene ring linked to each other with a single bond, the first benzene ring and the second benzene ring in the biphenyl structure being further linked to each other by cross-linking at at least one site other than the single bond, and
Condition (ii): a first linking structure including a benzene ring and a naphthalene ring linked to each other with a single bond, the benzene ring and the naphthalene ring in the first linking structure being each independently further fused or not fused with a monocyclic ring or fused ring, the benzene ring and the naphthalene ring in the first linking structure being further linked to each other by cross-linking at at least one site other than the single bond.

19. The composition according to claim 18, wherein at least one of the first compound or the second compound comprises, in a molecule, the structure of Condition (i) above.

20. The composition according to claim 19, wherein the first benzene ring and the second benzene ring in the biphenyl structure are further linked to each other by the cross-linking of Condition (i) above at one site other than the single bond.

21. The composition according to claim 19, wherein the cross-linking of Condition (i) above comprises a double bond.

22. The composition according to claim 19, wherein
the first benzene ring and the second benzene ring in the biphenyl structure are further linked to each other by the cross-linking of Condition (i) above at two sites other than the single bond, and
the cross-linking of Condition (i) above comprises no double bond.

23. The composition according to claim 18, wherein at least one of the first compound or the second compound comprises, in a molecule, a structure of Condition (ii) above.

24. The composition according to claim 23, wherein the cross-linking of Condition (ii) above comprises a double bond.

25. The composition according to claim 18, wherein the first compound and the second compound are each independently a compound selected from the group consisting of a compound represented by a formula (H11), a compound represented by a formula (H12), a compound represented by a formula (H13), a compound represented by a formula (H14), a compound represented by a formula (H15), and a compound represented by a formula (H16) below, 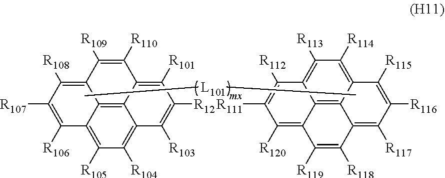
where, in the formula (H11):
R₁₀₁ to R₁₁₀ and R₁₁₁ to R₁₂₀ are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a substituted or unsubstituted aralkyl group having 7 to 50 carbon atoms, a group represented by —C(═O) R₈₀₁, a group represented by —COOR₈₀₂, a halogen atom, a cyano group, a nitro group, a substituted or unsubstituted aryl group having 6 to 50 ring carbon atoms, or a substituted or unsubstituted heterocyclic group having 5 to 50 ring atoms;
one of R₁₀₁ to R₁₁₀ represents a bonding position to L₁₀₁, and one of R₁₁₁ to R₁₂₀ represents a bonding position to L₁₀₁;
L₁₀₁ is a single bond, a substituted or unsubstituted arylene group having 6 to 24 ring carbon atoms, or a substituted or unsubstituted divalent heterocyclic group having 5 to 24 ring atoms;
mx is 0, 1, 2, 3, 4, or 5; and
when two or more L₁₀₁ are present, the two or more L₁₀₁ are mutually the same or different, 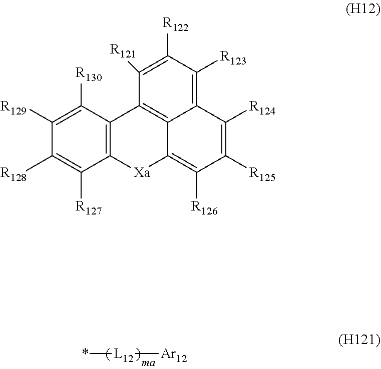
where, in the formula (H12):
Xa is an oxygen atom, a sulfur atom, C(R₁₂₀₁)(R₁₂₀₂), or Si(R₁₂₀₃)(R₁₂₀₄);
R₁₂₀₁ to R₁₂₀₄ are each independently a hydrogen atom, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a halogen atom, a cyano group, a nitro group, a substituted or unsubstituted aryl group having 6 to 50 ring carbon atoms, or a substituted or unsubstituted heterocyclic group having 5 to 50 ring atoms;
at least one combination of adjacent two or more of R₁₂₁ to R₁₃₀ are mutually bonded to form a substituted or unsubstituted monocyclic ring, mutually bonded to form a substituted or unsubstituted fused ring, or not mutually bonded;
R₁₂₁ to R₁₃₀ forming neither the substituted or unsubstituted monocyclic ring nor the substituted or unsubstituted fused ring are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a substituted or unsubstituted aralkyl group having 7 to 50 carbon atoms, a group represented by —C(═O)R₈₀₁, a group represented by —COOR₈₀₂, a halogen atom, a nitro group, a substituted or unsubstituted aryl group having 6 to 50 ring carbon atoms, a substituted or unsubstituted heterocyclic group having 5 to 50 ring atoms, or a group represented by a formula (H121) above;
at least one of R₁₂₁ to R₁₃₀ is a group represented by the formula (H121);
when a plurality of groups represented by the formula (H121) are present, the plurality of groups represented by the formula (H121) are mutually the same or different;
L₁₂ is a single bond, a substituted or unsubstituted arylene group having 6 to 50 ring carbon atoms, or a substituted or unsubstituted divalent heterocyclic group having 5 to 50 ring atoms;
ma is 0, 1, 2 or 3;
when two or more L₁₂ are present, the two or more L₁₂ are mutually the same or different;
Ar₁₂ is a substituted or unsubstituted aryl group having 6 to 50 ring carbon atoms, or a substituted or unsubstituted heterocyclic group having 5 to 50 ring atoms;
when two or more Ar₁₂ are present, the two or more Ar₁₂ are mutually the same or different; and
* in the formula (H121) represents a bonding position, 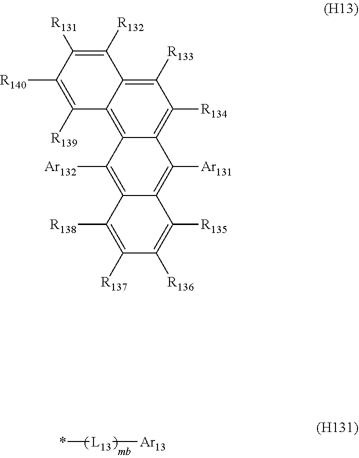
where, in the formula (H13):
R₁₃₁ to R₁₄₀, Ar₁₃₁, and Ar₁₃₂ are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a substituted or unsubstituted aralkyl group having 7 to 50 carbon atoms, a group represented by —C(═O) R₈₀₁, a group represented by —COOR₈₀₂, a halogen atom, a cyano group, a nitro group, a substituted or unsubstituted aryl group having 6 to 50 ring carbon atoms, a substituted or unsubstituted heterocyclic group having 5 to 50 ring atoms, or a group represented by a formula (H131) above;
at least one of R₁₃₁ to R₁₄₀, Ar₁₃₁, or Ar₁₃₂ is a group represented by the formula (H131);
when a plurality of groups represented by the formula (H131) are present, the plurality of groups represented by the formula (H131) are mutually the same or different;
L₁₃ is a single bond, a substituted or unsubstituted arylene group having 6 to 50 ring carbon atoms, or a substituted or unsubstituted divalent heterocyclic group having 5 to 50 ring atoms;
Ar₁₃ is a substituted or unsubstituted aryl group having 6 to 50 ring carbon atoms, or a substituted or unsubstituted heterocyclic group having 5 to 50 ring atoms;
mb is 0, 1, 2, 3, 4, or 5;
when two or more L₁₃ are present, the two or more L₁₃ are mutually the same or different;
when two or more Ar₁₃ are present, the two or more Ar₁₃ are mutually the same or different; and
* in the formula (H131) represents a bonding position to a benz[a]anthracene ring in the formula (H13), 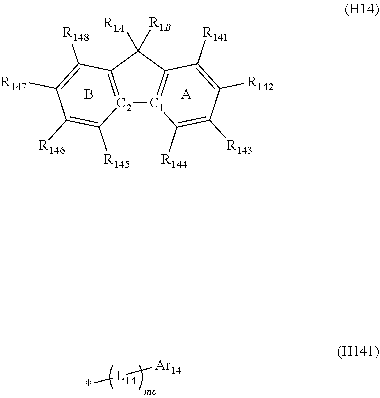
where, in the formula (H14):
R_{1A} and R_{1B} are each independently a substituted or unsubstituted alkyl group having 1 to 15 carbon atoms, a substituted or unsubstituted aryl group having 6 to 17 ring carbon atoms, or a substituted or unsubstituted heterocyclic group having 5 to 17 ring atoms;
at least one of R_{1A} or R_{1B} is a substituted or unsubstituted alkyl group having 1 to 15 carbon atoms;
at least one combination of a combination of adjacent two or more of R₁₄₁ to R₁₄₄ and a combination of adjacent two or more of R₁₄₅ to R₁₄₈ are mutually bonded to form a substituted or unsubstituted monocyclic ring, or mutually bonded to form a substituted or unsubstituted fused ring;
when a substituted or unsubstituted monocyclic ring or a substituted or unsubstituted fused ring is formed with the ring A, a group represented by a formula (H141) above is bonded to a carbon atom bonded to R₁₄₂ or, of carbon atoms forming the monocyclic ring with the ring A and the fused ring with the ring A, a carbon atom farthest from a carbon atom C₁ of the ring A, the carbon atom C₁ being bonded with a single bond to a carbon atom C₂ of a ring B;
when a substituted or unsubstituted monocyclic ring or a substituted or unsubstituted fused ring is formed with not the ring A but the ring B, a group represented by the formula (H141) is bonded to a carbon atom bonded to R₁₄₂; and
R₁₄₂ not being the group represented by the formula (H141), and R₁₄₁, R₁₄₃, R₁₄₄, and R₁₄₅ to R₁₄₈ forming neither the substituted or unsubstituted monocyclic ring nor the substituted or unsubstituted fused ring are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a substituted or unsubstituted aralkyl group having 7 to 50 carbon atoms, a group represented by —C(═O) R₈₀₁, a group represented by —COOR₈₀₂, a halogen atom, a cyano group, a nitro group, a substituted or unsubstituted aryl group having 6 to 17 ring carbon atoms, or a substituted or unsubstituted heterocyclic group having 5 to 17 ring atoms,
in the formula (H141):
Ar₁₄ is a substituted or unsubstituted aryl group having four or more fused rings or a substituted or unsubstituted heterocyclic group having four or more fused rings;
L₁₄ is a single bond, a substituted or unsubstituted arylene group having 6 to 17 ring carbon atoms, or a substituted or unsubstituted divalent heterocyclic group having 5 to 17 ring atoms;
mc is 0, 1, or 2;
* represents a bonding position to an atom forming a ring of the formula (H14); and
the compound represented by the formula (H14) does not have, in a molecule, three or more groups of a substituted or unsubstituted aryl group having four or more fused rings and a substituted or unsubstituted heterocyclic group having four or more fused rings, 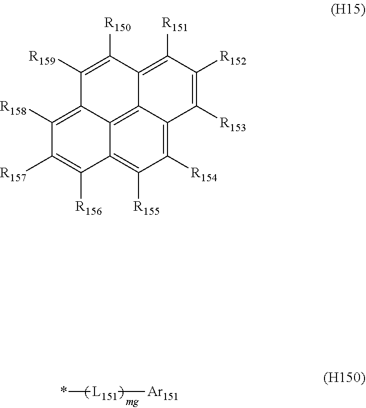
where, in the formula (H15):
R₁₅₀ to R₁₅₉ are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a substituted or unsubstituted aralkyl group having 7 to 50 carbon atoms, a group represented by—C(═O) R₈₀₁, a group represented by —COOR₈₀₂, a halogen atom, a cyano group, a nitro group, a substituted or unsubstituted aryl group having 6 to 50 ring carbon atoms, a substituted or unsubstituted heterocyclic group having 5 to 50 ring atoms, or a group represented by a formula (H150) above;
at least one of R₁₅₀ to R₁₅₉ is a group represented by the formula (H150);
when a plurality of groups represented by the formula (H150) are present, the plurality of groups represented by the formula (H150) are mutually the same or different;
L₁₅₁ is a single bond, a substituted or unsubstituted arylene group having 6 to 50 ring carbon atoms, or a substituted or unsubstituted divalent heterocyclic group having 5 to 50 ring atoms;
Ar₁₅₁ is a substituted or unsubstituted aryl group having 6 to 50 ring carbon atoms, or a substituted or unsubstituted heterocyclic group having 5 to 50 ring atoms;
mg is 0, 1, 2, 3, 4, or 5;
when two or more L₁₅₁ are present, the two or more L₁₅₁ are mutually the same or different;
when two or more Ar₁₅₁ are present, the two or more Ar₁₅₁ are mutually the same or different; and
* in the formula (H150) represents a bonding position to a pyrene ring in the formula (H15), 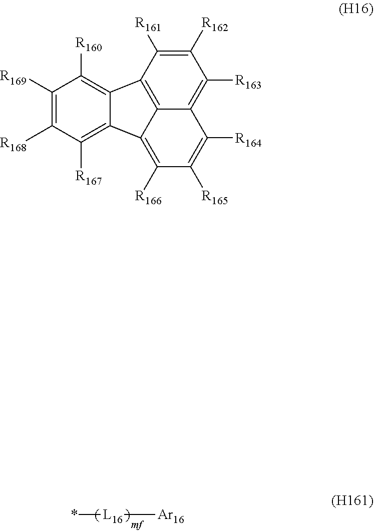
where, in the formula (H16):
at least one combination of adjacent two or more of R₁₆₀ to R₁₆₉ are mutually bonded to form a substituted or unsubstituted monocyclic ring, mutually bonded to form a substituted or unsubstituted fused ring, or not mutually bonded;
R₁₆₀ to R₁₆₉ forming neither the substituted or unsubstituted monocyclic ring nor the substituted or unsubstituted fused ring are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a substituted or unsubstituted aralkyl group having 7 to 50 carbon atoms, a group represented by —C(═O)R₈₀₁, a group represented by —COOR₈₀₂, a halogen atom, a cyano group, a nitro group, a substituted or unsubstituted aryl group having 6 to 50 ring carbon atoms, a substituted or unsubstituted heterocyclic group having 5 to 50 ring atoms, or a group represented by a formula (H161) above;
at least one of a substituent, if present, for the substituted or unsubstituted monocyclic ring, a substituent, if present, for the substituted or unsubstituted fused ring, or R₁₆₀ to R₁₆₉ is a group represented by the formula (H161);
when a plurality of groups represented by the formula (H161) are present, the plurality of groups represented by the formula (H161) are mutually the same or different;
L₁₆ is a single bond, a substituted or unsubstituted arylene group having 6 to 50 ring carbon atoms, or a substituted or unsubstituted divalent heterocyclic group having 5 to 50 ring atoms;
Ar₁₆ is a substituted or unsubstituted aryl group having 6 to 50 ring carbon atoms, or a substituted or unsubstituted heterocyclic group having 5 to 50 ring atoms;
mf is 0, 1, 2, 3, 4, or 5;
when two or more L₁₆ are present, the two or more L₁₆ are mutually the same or different;
when two or more Ar₁₆ are present, the two or more Ar₁₆ are mutually the same or different; and
* in the formula (H161) represents a bonding position to a ring represented by the formula (H16);
in the first compound and the second compound, R₉₀₁, R₉₀₂, R₉₀₃, R₉₀₄, R₉₀₅, R₉₀₆, R₉₀₇, R₈₀₁ and R₈₀₂ are each independently a hydrogen atom, a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms;
when a plurality of R₉₀₁ are present, the plurality of R₉₀₁ are mutually the same or different;
when a plurality of R₉₀₂ are present, the plurality of R₉₀₂ are mutually the same or different;
when a plurality of R₉₀₃ are present, the plurality of R₉₀₃ are mutually the same or different;
when a plurality of R₉₀₄ are present, the plurality of R₉₀₄ are mutually the same or different;
when a plurality of R₉₀₅ are present, the plurality of R₉₀₅ are mutually the same or different;
when a plurality of R₉₀₆ are present, the plurality of R₉₀₆ are mutually the same or different;
when a plurality of R₉₀₇ are present, the plurality of R₉₀₇ are mutually the same or different;
when a plurality of R₈₀₁ are present, the plurality of R₈₀₁ are mutually the same or different; and
when a plurality of R₈₀₂ are present, the plurality of R₈₀₂ are mutually the same or different.

26. The composition according to claim 25, wherein the first compound and the second compound are each independently a compound selected from the group consisting of a compound represented by a formula (H111), a compound represented by a formula (H122), a compound represented by a formula (H132), and a compound represented by a formula (H133) below, 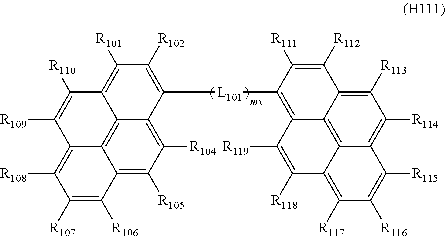
where, in the formula (H111):
R₁₀₁, R₁₀₂, R₁₀₄ to R₁₁₀, and R₁₁₁ to R₁₁₉ are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a substituted or unsubstituted aralkyl group having 7 to 50 carbon atoms, a group represented by —C(═O) R₈₀₁, a group represented by —COOR₈₀₂, a halogen atom, a cyano group, a nitro group, a substituted or unsubstituted aryl group having 6 to 50 ring carbon atoms, or a substituted or unsubstituted heterocyclic group having 5 to 50 ring atoms; and
L₁₀₁ and mx respectively represent the same as L₁₀₁ and mx in the formula (H11), 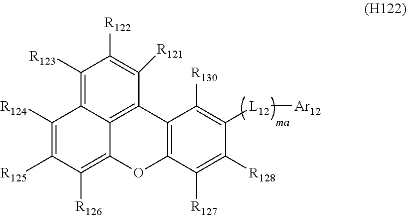
where, in the formula (H122):
R₁₂₁ to R₁₂₈ and R₁₃₀ are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a substituted or unsubstituted aralkyl group having 7 to 50 carbon atoms, a group represented by —C(═O)R₈₀₁, a group represented by —COOR₈₀₂, a halogen atom, a nitro group, a substituted or unsubstituted aryl group having 6 to 50 ring carbon atoms, or a substituted or unsubstituted heterocyclic group having 5 to 50 ring atoms; and
Ar₁₂, L₁₂, and ma respectively represent the same as Ar₁₂, L₁₂, and ma in the formula (H121), 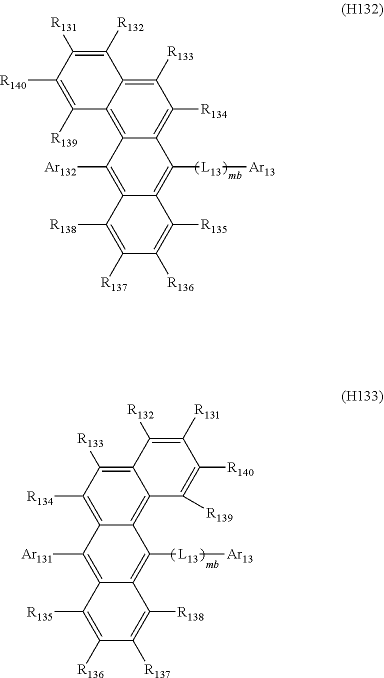
where, in the formulae (H132) and (H133):
R₁₃₁ to R₁₄₀, Ar₁₃₁, and Ar₁₃₂ are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a substituted or unsubstituted aralkyl group having 7 to 50 carbon atoms, a group represented by —C(═O) R₈₀₁, a group represented by —COOR₈₀₂, a halogen atom, a cyano group, a nitro group, a substituted or unsubstituted aryl group having 6 to 50 ring carbon atoms, or a substituted or unsubstituted heterocyclic group having 5 to 50 ring atoms; and
L₁₃, Ar₁₃, and mb respectively represent the same as L₁₃, Ar₁₃, and mb in the formula (H131).

27. The composition according to claim 25, wherein the group represented by the formula (H150) is a group represented by a formula (H151) below, 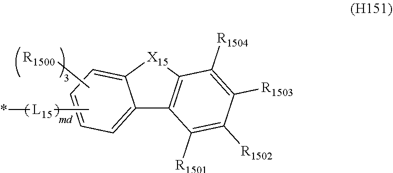
where, in the formula (H151):
X₁₅ is an oxygen atom or a sulfur atom;
L₁₅ is a single bond, a substituted or unsubstituted arylene group having 6 to 50 ring carbon atoms, or a substituted or unsubstituted divalent heterocyclic group having 5 to 50 ring atoms;
md is 0, 1, 2, 3, 4, or 5;
when two or more Lis are present, the two or more L₁₅ are mutually the same or different;
at least one combination of adjacent two or more of R₁₅₀₀ to R₁₅₀₄ are mutually bonded to form a substituted or unsubstituted monocyclic ring, mutually bonded to form a substituted or unsubstituted fused ring, or not mutually bonded;
R₁₅₀₀ to R₁₅₀₄ forming neither the substituted or unsubstituted monocyclic ring nor the substituted or unsubstituted fused ring are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a substituted or unsubstituted aralkyl group having 7 to 50 carbon atoms, a group represented by —C(═O) R₈₀₁, a group represented by —COOR₈₀₂, a halogen atom, a cyano group, a nitro group, a substituted or unsubstituted aryl group having 6 to 50 ring carbon atoms, or a substituted or unsubstituted heterocyclic group having 5 to 50 ring atoms;
a plurality of R₁₅₀₀ are mutually the same or different; and
* in the formula (H151) represents a bonding position to the pyrene ring in the formula (H15).

28. The composition according to claim 18, wherein each of the first compound and the second compound comprises, in a molecule, neither a biscarbazole structure nor an amine structure.

29-43. (canceled)
